(19) United States
(12) Patent Application Publication
(10) Pub. No.: US 2026/0078103 A1
(43) Pub. Date: Mar. 19, 2026

(54) ISOQUINOLONES AS PI3K INHIBITORS

(71) Applicant: OnKure, Inc., Boulder, CO (US)

(72) Inventors: James F. BLAKE, Boulder, CO (US); Mark Laurence BOYS, Boulder, CO (US); David A. MARESKA, Boulder, CO (US); Joshua Nathaniel PAYETTE, Boulder, CO (US); Christie A. SCHULTE, Boulder, CO (US); Bryan YESTREPSKY, Boulder, CO (US); Qian ZHAO, Boulder, CO (US)

(73) Assignee: OnKure, Inc., Boulder, CO (US)

(21) Appl. No.: 19/107,476

(22) PCT Filed: Sep. 6, 2023
(86) PCT No.: PCT/US2023/032033

§ 371 Date: Feb. 27, 2025

Related U.S. Application Data
(60) Provisional application No. 63/404,715, filed on Sep. 8, 2022.
(60) Provisional application No. 63/525,460, filed on Jul. 7, 2023.

(51) Int. Cl. C07D 401/04; A61K 31/4725; A61K 31/496; A61K 31/497; A61K 31/4985; A61K 31/4995; A61K 31/501; A61K 31/506; A61K 31/519; A61K 31/5377; A61K 31/541; A61K 31/675; C07D 217/24; C07D 401/12; C07D 401/14; C07D 405/04; C07D 405/14; C07D 413/04; C07D 413/14; C07D 417/04; C07D 417/14; C07D 451/02; C07D 471/04; C07D 471/08; C07D 471/10; C07D 487/04; C07D 487/08; C07D 487/10; C07D 491/048; C07D 491/107; C07D 498/04; C07D 513/04; C07F 9/6584

(52) U.S. Cl. CPC C07D 401/04; A61K 31/4725; A61K 31/496; A61K 31/497; A61K 31/4985; A61K 31/4995; A61K 31/501; A61K 31/506; A61K 31/519; A61K 31/5377; A61K 31/541; A61K 31/675; C07D 217/24; C07D 401/12; C07D 401/14; C07D 405/04; C07D 405/14; C07D 413/04; C07D 413/14; C07D 417/04; C07D 417/14; C07D 451/02; C07D 471/04; C07D 471/08; C07D 471/10; C07D 487/04; C07D 487/08; C07D 487/10; C07D 491/048; C07D 491/107; C07D 498/04; C07D 513/04; C07F 9/6584

(57) ABSTRACT

Novel PI3K inhibitors of the general formula (1) are described along with methods of their preparation and their use in the treatment of diseases associated with the elevation or activation of the PI3K pathway,

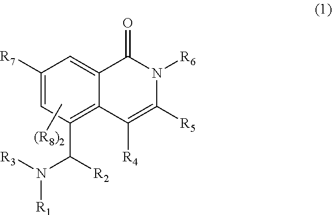

wherein R₁ to R₈ are as defined.

### CROSS-REFERENCE TO RELATED APPLICATIONS

This application claims priority to U.S. Provisional Application No. 63/525,460, filed Jul. 7, 2023; and U.S. Provisional Application No. 63/404,715, filed Sep. 8, 2022, the disclosures of which are incorporated in their entireties for all purposes.

### BACKGROUND OF THE INVENTION

Phosphatidylinositol lipids (Pls) and their various phosphorylated subspecies are second messengers involved in a wide array of cellular vesicle trafficking and signal transduction processes. Phosphoinositide 3′ kinases (PI3Ks) are a family of enzymes responsible for phosphorylation of the 3′ hydroxyl position of the inositol ring of Pls. PI3Ks are subdivided into 3 classes according to their structure and substrates. Class II PI3Ks (PI3K-C2α, PI3K-C2β, PI3K-C2γ) and Class III PI3Ks (vps34) are monomeric enzymes primarily associated with endocytosis and autophagy (Posor et al., Biochim Biophys Acta 2015, 1851, 794; Backer, Biochem J. 2016, 473, 2251). The Class I PI3Ks are heterodimeric, consisting of a catalytic kinase subunit (p110α, β, γ, δ) and one of several regulatory subunits that determine binding partners and subcellular localization. Class I PI3Ks are activated upon interaction with receptor tyrosine kinases (RTKs), Ras-related GTPases, G-protein coupled receptors, and/or related adaptor proteins, and in their active form convert phosphatidylinositol 4,5-diphosphate (PIP2) to phosphatidyl 3,4,5-triphosphate (PIP3) (Fruman et al., Cell 2017, 170, 605).

High local concentrations of PIP3 promote the recruitment and activation of downstream signaling partners, including AKT and mTOR. Activation of the AKT/mTOR pathways are implicated in several growth-related roles and pathologies including glucose regulation, cell survival, angiogenesis, and proliferation (Porta et al., Front Oncol. 2014, 4, 1), indicating a role for Class I PI3Ks as a critical upstream regulator of these functions.

Class I PI3Ks are further subdivided into 4 isoforms (α, β, γ, and δ) based on the identity of their catalytic (p110α, p110β, p110γ, or p110δ) and regulatory (p85α or its various splice variants, p85β, p55γ, or p101) subunits, giving rise to distinct roles in cellular physiology (Vanhaesebroeck et al., J Mol Med (Berl). 2016, 94, 5). PI3Kγ and PI3Kδ are mostly expressed in leukocytes and play an important role in pro-inflammatory pathways (Hawkins et. al., Biochimica et Biophysica Acta 2015, 1851, 882; Okkenhaug et al., Science 2002, 297, 1031; Ali et al., Nature 2004, 431, 1007). PI3Kα and β are more ubiquitously expressed and share similar but not identical roles. For example, PI3Kα has a nonredundant role in angiogenesis (Soler et al., J Exp Med. 2013, 210, 1937), while PI3Kβ is known to serve a specific function in platelet aggregation (Liu et. al., Nat Rev Drug Discov. 2009, 8, 627; Jackson et al., Nat Med. 2005, 11, 507).

Elevation or constitutive activation of the PI3K pathway is one of the most frequent events in human cancers. The PI3K pathway is overactivated through a variety of mechanisms, including activating mutation of PI3K isoforms, up-regulation of PI3K isoforms, loss or inactivation of the tumor suppressor PTEN, or hyperactivation of tyrosine kinase growth factor receptors or other upstream signaling partners (Yang et al., Mol Cancer 2019, 18, 1). Mutations in the gene coding for PI3Kα or mutations which lead to upregulation of PI3Kα have been found to occur in many human cancers such as lung, stomach, endometrial, ovarian, bladder, breast, colon, brain, prostate, and skin cancers (Goncalves et al., N Eng J Med. 2018, 379,2052). In particular, PIK3CA, the gene encoding the p110α subunit of PI3Kα, is frequently mutated or amplified in a variety of tumor types. Missense mutations occur in all domains of p110α, but cluster in two ‘hot spots’, the most common being E542K and E545K in the helical domain, and H1047R in the kinase domain. Helical domain mutations reduce inhibition of p110α by p85 or facilitate direct interaction of p110α with insulin receptor substrate 1 (IRS1)37, whereas kinase domain mutations increase interaction of p110α with lipid membranes, concomitantly upregulating signaling events. (Thorpe et al., Nat Rev Cancer 2015, 15, 7).

The development of inhibitors for the PI3K pathway has been challenging due to the inability to achieve dosing sufficient for tumor suppression without adverse events. To date PI3K inhibitors in the clinic (alpelisib, buparlisib, copanlisib, duvelisib, idelalisib, pictilisib, taselisib, and others) have caused dose-dependent adverse events such as hyperglycemia, rash, fatigue, diarrhea, etc. (Jiang et al., Mol Biol Rep. 2020, 47, 4587) which are known on-target toxicities. Hyperglycemia is a result of the body not producing enough insulin or aberrant utilization. The pancreas regulates insulin release in response to changes in blood glucose levels, resulting in either glucose uptake by muscle and fat cells when insulin levels are high or gluconeogenesis by the liver when insulin levels are low. Tissue cellular response to insulin requires PI3K signaling through the ubiquitously expressed p110α sub-unit. As a result, pan-PI3K inhibition of the target disrupts glucose metabolism in tissues, leading to insulin resistance (Hopkins et al., Nature 2018, 560, 499). To mitigate adverse events, selective PI3K isoform inhibitors were developed. The severity of the adverse event is dependent on the select isoform, for example PI3Kα inhibitors are associated with hyperglycemia and rash due to the p110α sub-unit role in insulin response (Rugo et al., The Breast 2022, 61, 156). Similarly, use of a selective PI3Kδ inhibitor (idelalisib), where the p110δ sub-unit is highly expressed in immune cells, causes severe diarrhea and colitis. Inhibition with a dual inhibitor (taselisib), a potent PI3Kδ inhibitor possessing modest PI3Kα inhibition led to gastrointestinal (GI) side effects, but a highly selective and potent PI3Kδ inhibitor (umbralisib) reported no GI related adverse events (Gadkar et al., CPT Pharmacometrics Syst Pharmacol. 2021, 11, 616). Such amelioration of adverse events with highly isoform selective and potent inhibitors demonstrates that a strategy to mitigate toxicity by developing mutant selective isoform inhibitors is promising for decreasing the severity of toxicity. Furthermore, selective inhibition of the mutant PI3Kα isoform over wild type may suppress cancer signaling while having minimal effect on PI3K signaling in healthy cells bearing just wild type PI3Kα, leading to a reduction in the toxicities associated with nonselective PI3K inhibition (Castel et al., Nat Cancer 2021 2, 587).

There is currently an interest in developing PI3K inhibitors for cancer therapy (WO 2023/081209, WO 2023/078401, WO 2023/060262, WO 2023/056407, WO 2021/202964, WO 2023/159155). However, there is a continued need for novel potent and selective PI3K inhibitors, either as single agents or as combination therapies, in the treatment of cancer.

### SUMMARY OF THE INVENTION

An aspect of the invention is a compound of Formula (1)

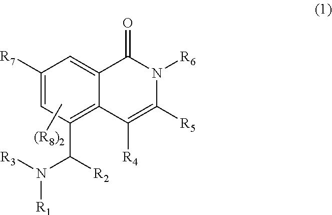

or a solvate, enantiomer, diastereomer, tautomer, polymorph or isotope-labeled compound thereof, or a pharmaceutically acceptable salt thereof,
wherein:
R₁ is alkyl, cycloalkyl, heterocyclyl, aryl or heteroaryl, where each of the alkyl, cycloalkyl, heterocyclyl, aryl or heteroaryl is unsubstituted or substituted;
R₂ is H, C₁-C₄ alkyl, C₃-C₇ cycloalkyl, CF₃, CH₂F or CF₂H, and where R₂ is not H, the carbon atom attached to R₂ is a chiral center and exists as a (R)- and (S)-racemic mixture or as either the (R)- or (S)-enantiomer;
R₃ is H or C₁-C₄ alkyl;
R₄ is H, F, Cl or CH₃;
R₆ is H, C₁-C₄ alkyl, C₃-C₇ cycloalkyl, heteroaryl, CF₃, CH₂F or CF₂H;
R₇ is H, C₁-C₄ alkyl, C₃-C₇ cycloalkyl, halogen, CN, CF₃, OCF₃, CH₂F or CF₂H;
each R₈ is independently H, C₁-C₄ alkyl, C₃-C₇ cycloalkyl, halogen, CN, CF₃, OCF₃, CH₂F or CF₂H;
R₅ is
   H;
   halogen;
   —O-L₁-L₂-L₃-L₄-L₅-L₆-L₇-R₉;
   —S-L₁-L₂-L₃-L₄-L₅-L₆-L₇-R₉;
   —S(O)-L₁-L₂-L₃-L₅-L₆-L₇-R₉;
   —S(O)₂-L₁-L₂-L₃-L₅-L₆-L₇-R₉;
   —(NR₁₀)-L₁-L₂-L₃-L₄-L₅-L₆-L₇-R₉; or
   -L₈-L₉-L₁₀-L₁₁-L₁₂-R₁₄,
wherein:
each of L₁, L₂, L₃, L₆ and L₇ is independently (CHR₁₁), (CHR₁₁—O), (CHR₁₁—S), (C₃-C₇ cycloalkyl), (CH₂)₁₋₄ or a bond;
L₄ is C═O, C═S or a bond;
L₅ is NR₁₀, S, O or a bond;
R₉ is H, C(═O)R₁₂, C(═O)NR₁₂R₁₃, NR₁₂R₁₃, C(═O)OR₁₂, C₁-C₆ alkyl, C₁-C₆ fluoroalkyl, cycloalkyl, heterocyclyl, aryl or heteroaryl, where each of the C₁-C₆ alkyl, C₁-C₆ fluoroalkyl, cycloalkyl, heterocyclyl, aryl or heteroaryl is unsubstituted or substituted; or alternatively when NR₁₀ is present, R₉ and R₁₀ together with the attached nitrogen atom may form a substituted or unsubstituted ring. In an exemplary embodiment, the ring is 4- to 7-membered substituted or unsubstituted non-aromatic heterocyclic ring containing (in addition to the nitrogen atom) 0, 1 or 2 heteroatoms which may be N, O, S or Si, with the proviso that if the ring size is 4 or 5, the number of additional heteroatoms will be 0 or 1 and if the ring size is from 6 to 7, the number of additional heteroatoms will be 0, 1 or 2, where if the ring is substituted, the substituents include, but are not limited to, one or more of CH₃, F, Cl, CF₃, CF₂H, CH₂F, OCH₃, cyclopropyl, CH₂CF₃, an oxetane ring, or CORₐ where Rₐ is C₁-C₄ alkyl, O—C₁-C₄ alkyl, or NR_{b}R_{c} where R_{b} and R_{c} are independently H or C₁-C₄ alkyl;
each of R₁₀ and R₁₁ is independently H or C₁-C₄ alkyl (such as CH₃, CH₂CH₃ or CH(CH₃)₂), where the C₁-C₄ alkyl is unsubstituted or substituted;
each of R₁₂ and R₁₃ is independently H, C₁-C₆ alkyl, cycloalkyl, heterocyclyl, aryl or heteroaryl, where each of the C₁-C₆ alkyl, cycloalkyl, heterocyclyl, aryl or heteroaryl is unsubstituted or substituted; or alternatively, R₁₂ and R₁₃ together with the attached nitrogen atom may form a substituted or unsubstituted ring. In an exemplary embodiment, the ring is 4- to 7-membered substituted or unsubstituted non-aromatic heterocyclic ring containing (in addition to the nitrogen atom) 0, 1 or 2 heteroatoms which may be N, O, S or Si, with the proviso that if the ring size is 4 or 5, the number of additional heteroatoms will be 0 or 1 and if the ring size is from 6 to 7, the number of additional heteroatoms will be 0, 1 or 2, where if the ring is substituted, the substituents include, but are not limited to, one or more of CH₃, F, Cl, CF₃, CF₂H, CH₂F, OCH₃, cyclopropyl, CH₂CF₃, an oxetane ring, or CORₐ where Rₐ is C₁-C₄ alkyl, O—C₁-C₄ alkyl, or NR_{b}R_{c} where R_{b} and R_{c} are independently H or C₁-C₄ alkyl;
L₈ is (CHR₁₅), (CHR₁₅—O), (CHR₁₅—S), (CHR₁₅—NR₁₆), C═O, C═S or a bond;
L₉ is C₃-C₇ cycloalkyl that is optionally part of a bridged, fused or spiro ring system, C(R₁₅)═C(R₁₅), C≡C or a bond;
L₁₀ is independently (CHR₁₅), O, S, (NCR₁₅), N(C═O) or a bond;
L₁₁ is (CHR₁₅), C═O, C═S or a bond;
L₁₂ is H, (C₃-C₇ cycloalkyl), heterocyclyl, aryl, heteroaryl or a bond, where each of the (C₃-C₇ cycloalkyl), heterocyclyl, aryl or heteroaryl is unsubstituted or substituted, and the (C₃-C₇ cycloalkyl) and/or heterocyclyl is optionally part of a bridged, fused or spiro ring system;
R₁₄ is H, CR₁₅R₁₆R₁₇, OR₁₇, SR₁₇, NR₁₆R₁₇, C₁-C₆ alkyl, C₁-C₆ fluoroalkyl, cycloalkyl, heterocyclyl, aryl or heteroaryl, where each of the C₁-C₆ alkyl, C₁-C₆ fluoroalkyl, cycloalkyl, heterocyclyl, aryl or heteroaryl is unsubstituted or substituted,
each of R₁₅ and R₁₆ is independently H or C₁-C₃ alkyl; and
each R₁₇ is independently H, C₁-C₆ alkyl, C₁-C₆ fluoroalkyl, cycloalkyl, heterocyclyl, aryl or heteroaryl, where each of the C₁-C₆ alkyl, C₁-C₆ fluoroalkyl, cycloalkyl, heterocyclyl, aryl or heteroaryl is unsubstituted or substituted; or alternatively, R₁₆ and R₁₇ together with the attached nitrogen atom may form a substituted or unsubstituted ring. In an exemplary embodiment, the ring is 4- to 7-membered substituted or unsubstituted non-aromatic heterocyclic ring containing (in addition to the nitrogen atom) 0, 1 or 2 heteroatoms which may be N, O or S, with the proviso that if the ring size is 4 or 5, the number of additional heteroatoms will be 0 or 1 and if the ring size is from 6 to 7, the number of additional heteroatoms will be 0, 1 or 2, where if the ring is substituted, the substituents include, but are not limited to, one or more of Me, F, Cl, CF₃, CF₂H, CH₂F, OCH₃, cyclopropyl, CH₂CF₃, an oxetane ring, or CORₐ where Rₐ is C₁-C₄ alkyl, O—C₁-C₄ alkyl, or NR_{b}R_{c} where R_{b} and R_{c} are independently H or C₁-C₄ alkyl;
with the proviso that when R₅ is -L₈-L₉-L₁₀-L₁₁-L₁₂-R₁₄, at least one of L₈, L₉, L₁₀, L₁₁, L₁₂ and R₁₄ is a carbon-containing moiety and R₅ is directly attached to the (isoquinolone) core structure by a carbon atom;
or R₅ is
a non-aromatic N-linked heterocyclic ring

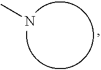

where the heterocyclic ring is substituted or unsubstituted, optionally contains one or more additional ring atoms selected from N, O, Si and S, and is optionally part of a bridged, fused or spiro ring system. In particular embodiments, the N-linked heterocyclyl ring is a substituted or unsubstituted aziridine, azetidine, pyrrolidine, imidazoline, imidazolidine, piperazine, morpholine, thiomorpholine, piperidine, indoline, tetrahydroquinoline, decahydroquinoline, 2-oxa-7-azaspiro[3.5]nonane, 1,4-dioxa-7-azaspiro[4.4]nonane or 2-azaadamantane.

In an exemplary embodiment, R₅ is —(NR₁₀)-L₁-L₂-L₃-L₄-L₅-L₆-L₇-R₉, where L₁ to L₇, R₉ and R₁₀ are as defined.

In an exemplary embodiment, R₅ is —O-L₁-L₂-L₃-L₄-L₅-L₅-L₇-R₉, where L₁ to L₇ and R₉ are as defined.

In an exemplary embodiment, R₅ is —S-L₁-L₂-L₃-L₄-L₅-L₆-L₇-R₉; —S(O)-L₁-L₂-L₃-L₅-L₆-L₇-R₉; or —S(O)₂-L₁-L₂-L₃-L₅-L₆-L₇-R₉, where L₁ to L₇ and R₉ are as defined.

In an exemplary embodiment, R₉ is a 6-membered aryl ring; or is a 5- to 6-membered heteroaryl ring containing from 1-3 nitrogen atoms; or is a non-aromatic 3- to 7-membered carbocycle; or is a non-aromatic 3- to 7-membered heterocycle containing from 1 to 3 heteroatoms selected from N, O, S and Si with the proviso that if the ring size is 4 or 5 then the number of heteroatoms will be 1 or 2 and if the ring size is 6 or 7 the number heteroatoms will be 1, 2 or 3; or is a C₁-C₆ alkyl group, where the aryl ring, the heteroaryl ring, the carbocycle, the heterocycle and the C₁-C₆ alkyl group are unsubstituted or substituted with one or more of CH₃, F, Cl, CF₃, CF₂H, CH₂F, OCH₃, —CH₂CF₃, cyclopropyl, —CN, N(CH₃)₂, an oxetane ring, a phenyl or phenoxy group optionally substituted with from 1 to 3 halogens (F, Cl or Br) or CH₃ groups, or CORₐ where Rₐ is C₁-C₄alkyl, O—C₁-C₄ alkyl or NR_{b}R_{c}, where R_{b} and R_{c} are independently H or C₁-C₄ alkyl.

In an exemplary embodiment of R₅, each of L₈, L₁₀ and L₁₁ is a bond and L₉ is not a bond.

In an exemplary embodiment of R₅, each of L₈, L₁₀ and L₁₁ is a bond and L₉ is cycloalkyl that is optionally part of a bridged, fused or spiro ring system.

In an exemplary embodiment of R₅, each of L₃, L₁₀ and L₁₁ is a bond and L₉ is cycloalkyl that is part of a bridged ring system.

In an exemplary embodiment of R₅, each of L₈, L₁₀ and L₁₁ is a bond and L₉ is cycloalkyl that is part of a fused ring system.

In an exemplary embodiment of R₅, each of L₈, L₁₀ and L₁₁ is a bond and L₉ is cycloalkyl that is part of a spiro ring system.

In an exemplary embodiment of R₅, each of L₈, L₁₀ and L₁₁ is a bond and L₉ is C(R₁₅)═C(R₁₅).

In an exemplary embodiment of R₅, each of L₈, L₁₀ and L₁₁ is a bond and L₉ is C≡C.

In an exemplary embodiment of R₅, each of L₈, L₉, L₁₀ and L₁₁ is a bond and L₁₂ is cycloalkyl, heterocyclyl, aryl or heteroaryl, where each of the cycloalkyl, heterocyclyl, aryl or heteroaryl is unsubstituted or substituted.

In an exemplary embodiment of R₅, each of L₈, L₉, L₁₀ and L₁₁ is a bond and L₁₂ is cycloalkyl, heterocyclyl, aryl or heteroaryl, where each of the cycloalkyl, heterocyclyl, aryl or heteroaryl is unsubstituted or substituted, and R₁₄ is H.

In an exemplary embodiment of R₅, each of L₈, L₉, L₁₀ and L₁₁ is a bond and L₁₂ is cycloalkyl, heterocyclyl, aryl or heteroaryl, where each of the cycloalkyl, heterocyclyl, aryl or heteroaryl is unsubstituted or substituted, and R₁₄ is cycloalkyl, heterocyclyl, aryl or heteroaryl, where each of the C₁-C₆ alkyl, cycloalkyl, heterocyclyl, aryl or heteroaryl is unsubstituted or substituted.

In an exemplary embodiment of R₅, each of L₈, L₉, L₁₀ and L₁₁ is a bond and L₁₂ is cycloalkyl, heterocyclyl, aryl or heteroaryl, where each of the cycloalkyl, heterocyclyl, aryl or heteroaryl is unsubstituted or substituted, and R₁₄ is —CR₁₄R₁₅R₁₆.

In an exemplary embodiment of R₅, each of L₈, L₉, L₁₀ and L₁₁ is a bond and L₁₂ is cycloalkyl, heterocyclyl, aryl or heteroaryl, where each of the cycloalkyl, heterocyclyl, aryl or heteroaryl is unsubstituted or substituted, and R₁₄ is —OR₁₆ or —OR₁₇.

In an exemplary embodiment of R₅, each of L₈, L₉, L₁₀ and L₁₁ is a bond and L₁₂ is cycloalkyl, heterocyclyl, aryl or heteroaryl, where each of the cycloalkyl, heterocyclyl, aryl or heteroaryl is unsubstituted or substituted, and R₁₄ is —SR₁₇.

In an exemplary embodiment of R₅, each of L₈, L₉, L₁₀ and L₁₁ is a bond and L₁₂ is cycloalkyl, heterocyclyl, aryl or heteroaryl, where each of the cycloalkyl, heterocyclyl, aryl or heteroaryl is unsubstituted or substituted, and R₁₄ is —NR₁₆R₁₇.

In an exemplary embodiment, R₅ is

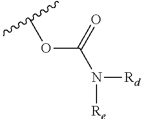

where Rₐ is H or CH₃ and Rₑ is CH₃, C₃-C₆ cycloalkyl, a six-membered aromatic or heteroaromatic ring containing from 0, 1 or 2 nitrogen atoms which may be optionally substituted with CH₃, F, Cl, CF₃, CF₂H, CH₂F, OCH₃, cyclopropyl, CN or N(CH₃)₂, or Rₐ and Rₑ together with the attached nitrogen atom may form a 4- to 7-membered non-aromatic heterocycle containing from 1 to 2 heteroatoms which may be either N or O, with the proviso that if the ring size is 4 or 5 the number of heteroatoms will be 1 and if the ring size is from 6 to 7, the number of heteroatoms will be 1 or 2, where the ring is unsubstituted or is substituted with one or more that includes, but is not limited to, CH₃, F, Cl, CF₃, CF₂H, CH₂F, OCH₃, cyclopropyl, CH₂CF₃, an oxetane ring, or CORₐ where Rₐ is C₁-C₄ alkyl, O—C₁-C₄ alkyl, or NR_{b}R_{c} where R_{b} and R_{c} are independently H or C₁-C₄ alkyl.

In an exemplary embodiment, R₅ is a N-linked non-aromatic heterocyclyl ring

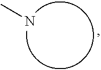

where the heterocyclyl ring is substituted or unsubstituted, optionally contains one or more additional atoms selected from N, O, Si and S, and is optionally part of a bridged, fused or spiro ring system. In particular embodiments, the N-linked heterocyclyl ring is azetidine, pyrrolidine, imidazoline, imidazolidine, piperazine, morpholine, thiomorpholine, piperidine, indoline, tetrahydroquinoline, decahydroquinoline, 2-oxa-7-azaspiro[3.5]nonane, 1,3,8-triazaspiro[4.5]-decan-4-one, 1,4-dioxa-7-azaspiro[4.4]nonane.

In an exemplary embodiment, the N-linked non-aromatic heterocyclyl ring is substituted or unsubstituted, optionally contains one or more additional atoms selected from N, O, Si and S, and is not part of a bridged, fused or spiro ring system.

In an exemplary embodiment, the N-linked non-aromatic heterocyclyl ring is substituted or unsubstituted, optionally contains one or more additional atoms selected from N, O, Si and S, and is part of a bridged, fused or spiro ring system.

In an exemplary embodiment, the N-linked non-aromatic heterocyclyl ring is substituted or unsubstituted, does not contain additional atoms selected from N, O, Si and S, and is not part of a bridged, fused or spiro ring system.

In an exemplary embodiment, the N-linked non-aromatic heterocyclyl ring is substituted or unsubstituted, does not contain additional atoms selected from N, O, Si and S, and is part of a bridged, fused or spiro ring system.

In an exemplary embodiment, the N-linked non-aromatic heterocyclyl ring is substituted or unsubstituted, contains at least one sulfur ring atom, and is not part of a bridged, fused or spiro ring system.

In an exemplary embodiment, the N-linked non-aromatic heterocyclyl ring is substituted or unsubstituted, contains at least one sulfur ring atom, and is part of a bridged, fused or spiro ring system.

In an exemplary embodiment, the N-linked non-aromatic heterocyclyl ring is substituted or unsubstituted, contains at least one oxygen ring atom, and is not part of a bridged, fused or spiro ring system.

In an exemplary embodiment, the N-linked non-aromatic heterocyclyl ring is substituted or unsubstituted, contains at least one oxygen ring atom, and is part of a bridged, fused or spiro ring system.

In an exemplary embodiment, the N-linked non-aromatic heterocyclyl ring is substituted or unsubstituted, contains at least one additional nitrogen ring atom, and is not part of a bridged, fused or spiro ring system.

In an exemplary embodiment, the N-linked non-aromatic heterocyclyl ring is substituted or unsubstituted, contains at least one additional nitrogen ring atom, and is part of a bridged, fused or spiro ring system.

In an exemplary embodiment of the compound of Formula (1), R₁ is heterocyclyl, aryl or heteroaryl, where each of the heterocyclyl, aryl or heteroaryl is unsubstituted or substituted.

In an exemplary embodiment of the compound of Formula (1), R₁ is heterocyclyl, where the heterocyclyl is unsubstituted or substituted.

In an exemplary embodiment of the compound of Formula (1), R₁ is aryl, where the aryl is unsubstituted or substituted.

In an exemplary embodiment of the compound of Formula (1), R₁ is heteroaryl, where the heteroaryl is unsubstituted or substituted.

In an exemplary embodiment of the compound of Formula (1), R₂ is CH₃.

In an exemplary embodiment of the compound of Formula (1), R₃ is H.

In an exemplary embodiment of the compound of Formula (1), R₄ is H.

In an exemplary embodiment of the compound of Formula (1), R₅ is —O-L₁-L₂-L₃-L₄-L₅-L₆-L₇-R₉.

In an exemplary embodiment of the compound of Formula (1), R₅ is —S-L₁-L₂-L₃-L₄-L₅-L₆-L₇-R₉.

In an exemplary embodiment of the compound of Formula (1), R₅ is —S(O)-L₁-L₂-L₃-L₅-L₆-L₇-R₉.

In an exemplary embodiment of the compound of Formula (1), R₅ is —S(O)₂-L₁-L₂-L₃-L₅-L₆-L₇-R₉.

In an exemplary embodiment of the compound of Formula (1), R₅ is —(NR₁₀)-L₁-L₂-L₃-L₄-L₅-L₆-L₇-R₉.

In an exemplary embodiment of the compound of Formula (1), R₅ is -L₅-L₉-L₁₀-L₁₁-L₁₂-R₁₄.

In an exemplary embodiment of the compound of Formula (1), R₆ is CH₃.

In an exemplary embodiment of the compound of Formula (1), R₇ is CH₃.

In an exemplary embodiment of the compound of Formula (1), R₈ is H.

In an exemplary embodiment of the compound of Formula (1), R₁ is heterocyclyl, where the heterocyclyl is unsubstituted or substituted, R₂ is CH₃ and R₃ is H.

In an exemplary embodiment of the compound of Formula (1), R₁ is aryl, where the aryl is unsubstituted or substituted, R₂ is CH₃ and R₃ is H.

In an exemplary embodiment of the compound of Formula (1), R₁ is heteroaryl, where the heteroaryl is unsubstituted or substituted, R₂ is CH₃ and R₃ is H.

In an exemplary embodiment of the compound of Formula (1), R₁ is heterocyclyl, where the heterocyclyl is unsubstituted or substituted, R₂ is CH₃, R₃ is Hand R₈ is H.

In an exemplary embodiment of the compound of Formula (1), R₁ is aryl, where the aryl is unsubstituted or substituted, R₂ is CH₃, R₃ is H and R₈ is H.

In an exemplary embodiment of the compound of Formula (1), R₁ is heteroaryl, where the heteroaryl is unsubstituted or substituted, R₂ is CH₃, R₃ is H and R₈ is H.

In an exemplary embodiment of the compound of Formula (1), R₁ is heterocyclyl, where the heterocyclyl is unsubstituted or substituted, R₂ is CH₃, R₃ is H, R₈ is H and R₆ is CH₃.

In an exemplary embodiment of the compound of Formula (1), R₁ is aryl, where the aryl is unsubstituted or substituted, R₂ is CH₃, R₃ is H, R₈ is H and R₆ is CH₃.

In an exemplary embodiment of the compound of Formula (1), R₁ is heteroaryl, where the heteroaryl is unsubstituted or substituted, R₂ is CH₃, R₃ is H, R₈ is H and R₆ is CH₃.

In an exemplary embodiment of the compound of Formula (1), R₁ is heterocyclyl, where the heterocyclyl is unsubstituted or substituted, R₂ is CH₃, R₃ is H and R₅ is —O-L₁-L₂-L₃-L₄-L₅-L₆-L₇-R₉.

In an exemplary embodiment of the compound of Formula (1), R₁ is aryl, where the aryl is unsubstituted or substituted, R₂ is CH₃, R₃ is H and R₅ is —S-L₁-L₂-L₃-L₄-L₅-L₆-L₇-R₉.

In an exemplary embodiment of the compound of Formula (1), R₁ is heteroaryl, where the heteroaryl is unsubstituted or substituted, R₂ is CH₃, R₃ is H and R₅ is —S(O)-L₁-L₂-L₃-L₅-L₆-L₇-R₉.

In an exemplary embodiment of the compound of Formula (1), R₁ is heterocyclyl, where the heterocyclyl is unsubstituted or substituted, R₂ is CH₃, R₃ is H and R₅ is —S(O)₂-L₁-L₂-L₃-L₅-L₆-L₇-R₉.

In an exemplary embodiment of the compound of Formula (1), R₁ is aryl, where the aryl is unsubstituted or substituted, R₂ is CH₃, R₃ is H and R₅ is —(NR₁₀)-L₁-L₂-L₃-L₄-L₅-L₆-L₇-R₉.

In an exemplary embodiment of the compound of Formula (1), R₁ is heteroaryl, where the heteroaryl is unsubstituted or substituted, R₂ is CH₃, R₃ is H and R₅ is -L₈-L₉-L₁₀-L₁₁-L₁₂-R₁₄.

In an exemplary embodiment of the compound of Formula (1), R₁ is heterocyclyl, where the heterocyclyl is unsubstituted or substituted, R₂ is CH₃, R₃ is H, R₅ is —O-L₁-L₂-L₃-L₄-L₅-L₆-L₇-R₉ and R₈ is H.

In an exemplary embodiment of the compound of Formula (1), R₁ is aryl, where the aryl is unsubstituted or substituted, R₂ is CH₃, R₃ is H, R₅ is —S-L₁-L₂-L₃-L₄-L₅-L₆-L₇-R₉ and R₈ is H.

In an exemplary embodiment of the compound of Formula (1), R₁ is heteroaryl, where the heteroaryl is unsubstituted or substituted, R₂ is CH₃, R₃ is H, R₅ is —S(O)-L₁-L₂-L₃-L₅-L₆-L₇-R₉ and R₈ is H.

In an exemplary embodiment of the compound of Formula (1), R₁ is heterocyclyl, where the heterocyclyl is unsubstituted or substituted, R₂ is CH₃, R₃ is H, R₅ is —S(O)₂-L₁-L₂-L₃-L₅-L₆-L₇-R₉ and R₈ is H.

In an exemplary embodiment of the compound of Formula (1), R₁ is aryl, where the aryl is unsubstituted or substituted, R₂ is CH₃, R₃ is H, R₅ is —(NR₁₀)-L₁-L₂-L₃-L₄-L₅-L₆-L₇-R₉ and R₈ is H. In an exemplary embodiment of the compound of Formula (1), R₁ is heteroaryl, where the heteroaryl is unsubstituted or substituted, R₂ is CH₃, R₃ is H, R₅ is -L₈-L₉-L₁₀-L₁₁-L₁₂-R₁₄ and R₈ is H.

In an exemplary embodiment of the compound of Formula (1), R₁ is heterocyclyl, where the heterocyclyl is unsubstituted or substituted, R₂ is CH₃, R₃ is H, R₅ is —O-L₁-L₂-L₃-L₄-L₅-L₆-L₇-R₉, R₈ is H and R₆ is CH₃.

In an exemplary embodiment of the compound of Formula (1), R₁ is aryl, where the aryl is unsubstituted or substituted, R₂ is CH₃, R₃ is H, R₅ is —S-L₁-L₂-L₃-L₄-L₅-L₅-L₇-R₉, R₈ is H and R₆ is CH₃.

In an exemplary embodiment of the compound of Formula (1), R₁ is aryl, where the aryl is unsubstituted or substituted, R₂ is CH₃, R₃ is H, R₅ is —S(O)-L₁-L₂-L₃-L₄-L₅-L₆-L₇-R₉, R₈ is H and R₆ is CH₃.

In an exemplary embodiment of the compound of Formula (1), R₁ is heteroaryl, where the heteroaryl is unsubstituted or substituted, R₂ is CH₃, R₃ is H, R₅ is —S(O)₂-L₁-L₂-L₃-L₅-L₆-L₇-R₉, R₈ is H and R₆ is CH₃.

In an exemplary embodiment of the compound of Formula (1), R₁ is heterocyclyl, where the heterocyclyl is unsubstituted or substituted, R₂ is CH₃, R₃ is H, R₅ is —(NR₁₀)-L₁-L₂-L₃-L₄-L₅-L₆-L₇-R₉, R₈ is H and R₆ is CH₃.

In an exemplary embodiment of the compound of Formula (1), R₁ is aryl, where the aryl is unsubstituted or substituted, R₂ is CH₃, R₃ is H, R₅ is -L₈-L₉-L₁₀-L₁₁-L₁₂-R₁₄, R₈ is H and R₆ is CH₃.s

In an exemplary embodiment, the compound of Formula (1) is a compound of Formula (2)

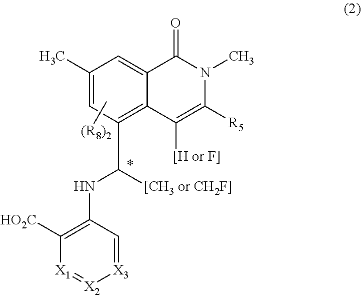

or a solvate, enantiomer, diastereomer, tautomer, polymorph or isotope-labeled compound, or a pharmaceutically acceptable salt thereof,
wherein:
each of X₁, X₂ and X₃ is independently N, CH or substituted C;
R₅ and R₈ are defined as in the compound of Formula (1), and
the carbon marked with * is a chiral center and exists as a (R)- and (S)-racemic mixture or as either the (R)- or (S)-enantiomer.

In an exemplary embodiment, the compound of Formula (1) is a compound of Formula (3)

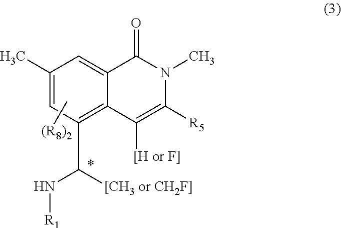

or a solvate, enantiomer, diastereomer, tautomer, polymorph or isotope-labeled compound, or a pharmaceutically acceptable salt thereof,
wherein:
R₁ is heterocyclyl, aryl or heteroaryl, wherein the heterocyclyl, aryl or heteroaryl ring directly attached to the nitrogen atom linked to the asymmetric center attached to the isoquinoline moiety contains a carboxylic acid substituent at the ortho position to the point of attachment and optionally contains one or more additional substituents,
R₅ and R₅ are defined as in the compound of Formula (1), and
the carbon marked with * is a chiral center and exists as a (R)- and (S)-racemic mixture or as either the (R)- or (S)-enantiomer.

In an exemplary embodiment of the compound of Formula (3), R₁ is heteroaryl.

In an exemplary embodiment of the compound of Formula (3), R₁ is aryl.

In an exemplary embodiment of the compound of Formula (3), R₁ is heterocyclyl and R₅ is —O-L₁-L₂-L₃-L₄-L₅-L₆-L₇-R₉.

In an exemplary embodiment of the compound of Formula (3), R₁ is heteroaryl and R₅ is —O-L₁-L₂-L₃-L₄-L₅-L₆-L₇-R₉.

In an exemplary embodiment of the compound of Formula (3), R₁ is aryl, where the aryl is unsubstituted or substituted and R₅ is —O-L₁-L₂-L₃-L₄-L₅-L₆-L₇-R₉.

In an exemplary embodiment of the compound of Formula (3), R₁ is heterocyclyl, where the heterocyclyl is unsubstituted or substituted and R₅ is —S-L₁-L₂-L₃-L₄-L₅-L₆-L₇-R₉.

In an exemplary embodiment of the compound of Formula (3), R₁ is heteroaryl, where the heteroaryl is unsubstituted or substituted and R₄ is —S-L₁-L₂-L₃-L₄-L₅-L₆-L₇-R₉.

In an exemplary embodiment of the compound of Formula (3), R₁ is aryl, where the aryl is unsubstituted or substituted and R₅ is —S-L₁-L₂-L₃-L₄-L₅-L₆-L₇-R₉.

In an exemplary embodiment of the compound of Formula (3), R₁ is heterocyclyl, where the heterocyclyl is unsubstituted or substituted and R₅ is —S(O)-L₁-L₂-L₃-L₄-L₅-L₆-L₇-R₉.

In an exemplary embodiment of the compound of Formula (3), R₁ is heteroaryl, where the heteroaryl is unsubstituted or substituted and R₅ is —S(O)-L₁-L₂-L₃-L₄-L₅-L₅-L₇-R₉.

In an exemplary embodiment of the compound of Formula (3), R₁ is aryl, where the aryl is unsubstituted or substituted and R₅ is —S(O)-L₁-L₂-L₃-L₄-L₅-L₆-L₇-R₉.

In an exemplary embodiment of the compound of Formula (3), R₁ is heterocyclyl, where the heterocyclyl is unsubstituted or substituted and R₅ is —S(O)₂-L₁-L₂-L₃-L₄-L₅-L₆-L₇-R₉.

In an exemplary embodiment of the compound of Formula (3), R₁ is heteroaryl, where the heteroaryl is unsubstituted or substituted and R₅ is —S(O)₂-L₁-L₂-L₃-L₄-L₅-L₆-L₇-R₉.

In an exemplary embodiment of the compound of Formula (3), R₁ is aryl, where the aryl is unsubstituted or substituted and R₅ is —S(O)₂-L₁-L₂-L₃-L₄-L₅-L₆-L₇-R₉.

In an exemplary embodiment of the compound of Formula (3), R₁ is heterocyclyl, where the heterocyclyl is unsubstituted or substituted and R₅ is —(NR₁₀)-L₁-L₂-L₃-L₄-L₅-L₆-L₇-R₉.

In an exemplary embodiment of the compound of Formula (3), R₁ is heteroaryl, where the heteroaryl is unsubstituted or substituted and R₅ is —(NR₁₀)-L₁-L₂-L₃-L₄-L₅-L₆-L₇-R₉.

In an exemplary embodiment of the compound of Formula (3), R₁ is aryl, where the aryl is unsubstituted or substituted and R₅ is —(NR₁₀)-L₁-L₂-L₃-L₄-L₅-L₆-L₇-R₉.

In an exemplary embodiment of the compound of Formula (3), R₁ is heterocyclyl, where the heterocyclyl is unsubstituted or substituted and R₅ is -L₈-L₉-L₁₀-L₁₁-L₁₂-R₁₄.

In an exemplary embodiment of the compound of Formula (3), R₁ is heteroaryl, where the heteroaryl is unsubstituted or substituted and R₅ is -L₃-L₉-L₁₀-L₁₁-L₁₂-R₁₄.

In an exemplary embodiment of the compound of Formula (3), R₁ is aryl, where the aryl is unsubstituted or substituted and R₅ is -Lg-L₉-L₁₀-L₁₁-L₁₂-R₁₄.

An aspect of the invention is a pharmaceutical composition comprising any compound of the invention as described herein (such as any one of Formula (1), (2), (3), (4), (5) or (6)) or a solvate, enantiomer, diastereomer, tautomer, polymorph or isotope-labeled compound thereof, or a pharmaceutically acceptable salt thereof, and a pharmaceutically acceptable carrier.

In an exemplary embodiment, the pharmaceutical composition comprising any compound of the invention as described herein (such as any one of Formula (1), (2), (3), (4), (5) or (6)) or a solvate, enantiomer, diastereomer, tautomer, polymorph or isotope-labeled compound thereof, or a pharmaceutically acceptable salt thereof further comprises one or more anti-cancer agents.

Another aspect of the invention is a method of treating a disease in which PI3K activity is implicated in a subject in need of such treatment, the method comprising administering to the subject a therapeutically effective amount of any compound of the invention as described herein (such as any one of Formula (1), (2), (3), (4), (5) or (6)) or a solvate, enantiomer, diastereomer, tautomer, polymorph or isotope-labeled compound thereof, or a pharmaceutically acceptable salt thereof.

In an exemplary embodiment, the disease to be treated is cancer. In a particular embodiment, the disease is a cancer bearing a PI3Kα H1047 mutation (such as H1047R).

### DETAILED DESCRIPTION OF THE INVENTION

The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which can be expressed as the ratio LD₅₀/ED₅₀. Compounds that exhibit large therapeutic indices are preferred. The data obtained from these cell culture assays and additional animal studies can be used in formulating a range of dosages for human use. The dosages of such compounds lie preferably within a range of circulating concentrations that include the ED₅₀ with little or no toxicity. The dosage varies within this range depending upon the dosage form employed, the sensitivity of the patient, and the route of administration.

The term “symptom” as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue body imaging scans and other medical testing results.

The term “disease” as used herein, refers to any impairment of the normal state of the living animal or one of its parts that interrupts or modifies the performance of the vital functions. Typically manifested by distinguishing signs and symptoms, a disease is usually a response to i) environmental factors (such as malnutrition, industrial hazards, or climate); ii) specific infective agents (such as worms, bacteria, or viruses); iii) inherent defects of the organism (such as genetic anomalies); and/or iv) combinations of these factors.

The terms “reduce”, “inhibit”, “diminish”, “suppress”, “decrease”, “prevent” and grammatical equivalents thereof (including “lower”, “smaller”, etc.) when used in reference to the expression of any symptom in an untreated subject relative to a treated subject, indicate that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “inhibitory compound” as used herein, refers to any compound capable of interacting with (i.e., for example, attaching, binding, etc.) to a binding partner under conditions such that the binding partner becomes unresponsive to its natural ligands. Inhibitory compounds may include, but are not limited to, small organic molecules, antibodies, and proteins/peptides.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like. A drug is attached to a medium (or carrier) if it is impregnated, incorporated, coated, in suspension with, in solution with, mixed with, etc.

The term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides, or sugars.

The term “administered” or “administering” as used herein, refers to any method of providing a composition to a patient such that the composition has its intended effect on the patient. An exemplary method of administering is by a direct mechanism such as, local tissue administration (i.e., for example, extravascular administration, such as subcutaneous, intramuscular, or intraperitoneal), intravenous, oral ingestion, transdermal patch, topical, inhalation, suppository, etc.

The term “patient” as used herein, is a human or animal and needs not be hospitalized. For example, out-patients and persons in nursing homes are “patients.” A patient may be a human or non-human animal of any age and therefore includes both adults and juveniles (i.e., children). It is not intended that the term “patient” connote a need for medical treatment. Therefore, a patient may voluntarily be subject to experimentation, whether clinical or in support of basic science studies.

The term “subject” as used herein, refers to, but is not limited to, humans (e.g., a male or female of any age group, e.g., a pediatric subject (e.g., infant, child, adolescent) or adult subject (e.g., young adult, middle-aged adult or senior adult)) and/or other primates (e.g., monkeys); non-human mammals, such as cows, pigs, horses, sheep, mice, goats, cats, dogs; and/or birds, such as chickens, ducks and/or geese.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

The term “derived from” as used herein, refers to the source of a compound or sequence. In one respect, a compound or sequence may be derived from an organism or particular species. In another respect, a compound or sequence may be derived from a larger complex or sequence.

The term “test compound” as used herein, refers to any compound or molecule considered a candidate as an inhibitory compound.

The term “combination therapy” as used herein refers to refers to a dosing regimen of two or more different therapeutically active agents during a period of time, wherein the therapeutically active agents are administered together or separately. In one embodiment the combination therapy is a non-fixed combination.

The term “non-fixed combination” as used herein refers to two or more different therapeutic agents that are formulated as separate compositions or dosages such that they may be administered separately to a subject in need thereof either simultaneously or sequentially with variable intervening time limits.

The term “synergy” or “synergistic” as used herein refers to the phenomenon where the combination of two therapeutic agents of a combination therapy is greater in terms of measured results than the sum of the effect of each agent when administered alone.

The term “in vivo” as used herein refers to an event that takes place in a subject's body.

The term “in vitro” as used herein refers to an event that takes places outside of a subject's body.

The term “protein” as used herein, refers to any of numerous naturally occurring extremely complex substances (such as an enzyme or antibody) that contain amino acid residues joined by peptide bonds, and which include carbon, hydrogen, nitrogen, oxygen, and typically sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “pharmaceutically acceptable” or “pharmacologically acceptable” as used herein, refers to molecular entities and compositions that do not produce adverse, allergic, or other untoward reactions when administered to an animal or a human.

The term, “pharmaceutically acceptable carrier” as used herein, includes any and all solvents, or a dispersion medium including, but not limited to, water, ethanol, a polyol (such as, for example, glycerol, propylene glycol, and liquid polyethylene glycol, and the like), suitable mixtures thereof, vegetable oils, coatings, isotonic and absorption delaying agents, liposome, commercially available cleansers, and the like. Supplementary bioactive ingredients also can be incorporated into such carriers.

The term “pharmaceutically acceptable salt” as used herein, refers to a salt that does not adversely impact the biological activity and properties of the compound and is suitable for use in contact with the tissues of subjects without undue toxicity, irritation and/or allergic response and the like. Pharmaceutically acceptable salts include those derived from suitable inorganic acids, organic acids and bases, and include hydrochloric acid, hydrobromic acid, phosphoric acid, sulfuric acid, nitric acid, acetic acid, oxalic acid, maleic acid, tartaric acid, citric acid, succinic acid, malonic acid, ascorbic acid, methanesulfonic acid, ethanesulfonic acid, benzenesulfonic acid, p-toluenesulfonic acid, benzoic acid, naphthalene sulfonic acid, lactic acid, succinic acid, oxalic acid, stearic acid, and the like. In some instances, pharmaceutically acceptable salts are obtained by reacting a compound having acidic group described herein with a base to form a salt such as an ammonium salt, an alkali metal salt (e.g., a sodium or a potassium salt), an alkaline earth metal salt (e.g., a calcium or a magnesium salt), a salt formed from an organic base, and an amino acid salt. Pharmaceutically acceptable salts derived from appropriate bases include alkali metals, alkaline earth metals, and ammonium and quaternary ammonium compounds. Specific metals include, but are not limited to, sodium, lithium, potassium, calcium, magnesium, iron, zinc, copper, manganese, aluminum, and the like. Organic bases from which salts may be prepared include, for example, primary, secondary, and tertiary amines.

The term “prodrug” as used herein, refers to a compound that is transformed in vivo to yield a disclosed compound or a pharmaceutically acceptable form of the compound. A prodrug may be inactive when administered to a subject, but is converted in vivo to an active compound. In various instances, a prodrug has improved physicochemical properties (such as bioavailability) and/or delivery properties over the parent compound. Prodrugs are typically designed to enhance pharmaceutically and/or pharmacokinetically based properties associated with the parent compound. The prodrug compound often offers advantages of solubility, tissue compatibility or delayed release in subject. Prodrugs include compounds wherein a hydroxy, amino, or mercapto group is bonded to any group that, when the prodrug is administered to a subject, cleaves to form a free hydroxy, free amino, or free mercapto group, respectively. Prodrugs are well known to be prepared from carboxylic acids in the form of, for example, carboxylate esters or thioesters.

The term, “purified” or “isolated” as used herein, may refer to a composition (such as, for example, a peptide composition) that has been subjected to treatment (e.g., fractionation) to remove various other components, and which composition substantially retains its expressed biological activity.

The term “sample” as used herein, includes, for example, environmental and biological samples. Environmental samples include material from the environment such as soil and water. Biological samples include animal (e.g., human), fluids (e.g., blood, plasma, and serum), solids (e.g., stool), tissue, liquid foods (e.g., milk), and solid foods (e.g., vegetables). For example, a pulmonary sample may be collected by bronchoalveolar lavage (BAL) which comprises fluid and cells derived from lung tissues. A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

The term “biologically active” as used herein, refers to any molecule having structural, regulatory or biochemical functions. For example, biological activity may be determined, for example, by restoration of wild-type growth in cells lacking protein activity. Cells lacking protein activity may be produced by many methods (i.e., for example, point mutation and frame-shift mutation). Complementation is achieved by transfecting cells which lack protein activity with an expression vector which expresses the protein, a derivative thereof, or a portion thereof.

The term “label” or “detectable label” as used herein, refers to any composition detectable by spectroscopic, photochemical, biochemical, immunochemical, electrical, optical or chemical means. Such labels include biotin for staining with labeled streptavidin conjugate, magnetic beads (e.g., Dynabeads*), fluorescent dyes (e.g., fluorescein, Texas Red*, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include, but are not limited to, U.S. Pat. Nos. 3,817,837; 3,850,752; 3,939,350; 3,996,345; 4,277,437; 4,275,149; and 4,366,241 (all herein incorporated by reference in their entireties). The labels contemplated in the present invention may be detected by conventional methods. For example, radiolabels may be detected using photographic film or scintillation counters, fluorescent markers may be detected using a photodetector to detect emitted light. Enzymatic labels are typically detected by providing the enzyme with a substrate and detecting, the reaction product produced by the action of the enzyme on the substrate, and calorimetric labels are detected by simply visualizing the colored label.

The term “conjugate” as used herein, refers to any compound that has been formed by the joining of two or more moieties.

A “moiety” or “group” as used herein, is any type of molecular arrangement designated by formula, chemical name, or structure. Within the context of certain embodiments, a conjugate comprises one or more moieties or chemical groups. This means that the formula of the moiety is substituted at some position in order to be joined and be a part of the molecular arrangement of the conjugate. Although moieties may be directly covalently joined, it is not intended that the joining of two or more moieties must be directly to each other. A linking group, a crosslinking group, or a joining group refers to any molecular arrangement that will connect moieties by covalent bonds such as, but not limited to, one or more amide group(s). Additionally, although the conjugate may be unsubstituted, the conjugate may have a variety of additional substituents connected to the linking groups and/or connected to the moieties.

A “polymer” or “polymer group” as used herein, refers to a chemical species or group composed of repeatedly linked moieties. Within certain embodiments, it is preferred that the number of repeating moieties is 3 or more or greater than 10. The linked moieties may be identical in structure or may vary in their moiety structures. A “monomeric polymer” or “homopolymer” is a polymer that contains the same repeating, asymmetric subunit. A “copolymer” is a polymer derived from two or more types of monomeric species (i.e., two or more different chemical asymmetric subunits). “Block copolymers” are polymers comprised of two or more species of polymer subunits linked by covalent bonds.

The term “substituted” as used herein, refers to at least one hydrogen atom of a molecular arrangement that is replaced with a non-hydrogen substituent. The number of substituents present depends on the number of hydrogen atoms available for replacement and includes replacement of more than one hydrogen atom bound to a single atom (such as in the case of a carbon atom or a silicon atom which may be available for mono-, di- or tri-substitution or in the case of a nitrogen atom which may be available for mono-, di- or tri-substitution or in the case of an oxygen atom or a sulfur atom which may be available for mono-substitution). In the case of an oxo substituent (“═O”), two hydrogen atoms are replaced (which provides, for example, —(CH₂)—C(═O)—CH₃ as a substituent when the two hydrogen atoms of the middle carbon atom of —CH₂—CH₂—CH₃ are replaced). When substituted, one or more of the groups below are “substituents.” Substituents include, but are not limited to, halogen (e.g., F, Cl, Br, I), hydroxy (OH), oxo, cyano (CN), nitro (NO₂), amino, alkylamino, dialkylamino, branched or unbranched alkyl (e.g., methyl, ethyl, propyl, isopropyl, sec-butyl, etc.), cycloalkyl (e.g., cyclopropyl), fluoroalkyl (e.g., CF₃, CF₂H, CH₂F, CH₂CF₃, CH₂CF₂H, CHFCHF₂, CF₂CH₂F, CF₂CF₃, CF₂CH₃, CF(CH₃)₂, CH₂CH₂CF₃, CF₂CH₂CF₃, CF₂CF₂CF₃, etc.) or more generally, haloalkyl (e.g., CH₂Cl, CH(CH₃) Br, etc.), O-alkyl (alkoxy) (e.g., OCH₃, OCH₂CH₃, OCH(CH₃)₂, etc.), O-cycloalkyl (e.g., O-cyclopropyl), O-haloalkyl (e.g., OCF₂H, OCFH₂, OCF₃, OCH₂CF₃, OCH₂CF₂H, OCHFCHF₂, OCF₂CH₂F, OCF₂CF₃, OCF₂CH₃, OCF(CH₃)₂, OCH₂CH₂CF₃, OCF₂CH₂CF₃, OCF₂CF₂CF₃ or OCH₂Cl), O-aryl (e.g., O-phenyl), O-heteroaryl, O-heterocyclyl, thioalkyl (e.g., S—CH₃), hydroxyalkyl (e.g., CH₂OH), alkyl ether (e.g., CH₂OCH₃), alkynyl (e.g., —C≡CR_{f}), alkenyl (e.g., —CR(═CR_{f}R_{g}), aryl (e.g., phenyl), arylalkyl (e.g., CH₂Ph), heteroaryl (e.g., pyridyl or any 5- or 6-membered heteroaryl ring), heteroarylalkyl (e.g., CH₂-pyridine), heterocyclyl, heterocycloalkyl and as well as, —NR_{f}R_{g}, —NR(C(═O)R_{g}, —NR(C(═O)NR_{f}NR_{g}, —NR_{f}—C(═O)OR(SO₂R_{g}, —C(═O)R_{f}, —C(═O)OR_{f}, —OR_{f}, —C(═O)NR_{f}R_{g}, —OC(═O)NR_{f}R_{g}, —SR_{f}, —SOR_{f}, —S(═O)₂R_{f}, —OS(═O)₂R_{f} and —S(═O)OR_{f}, where each R_{f} and R_{g} may be the same or different and are independently, hydrogen, alkyl (e.g., CH₃), substituted alkyl, haloalkyl, aryl, substituted aryl, arylalkyl, substituted arylalkyl, heterocyclyl, substituted heterocyclyl, heterocycloalkyl, substituted heterocycloalkyl, heteroaryl or substituted heteroaryl. In addition, the above substituents may be further substituted with one or more substituents as defined above, such that a substituent may constitute, for example, a substituted alkyl, a substituted aryl, a substituted arylalkyl, a substituted heterocyclyl, or a substituted heterocycloalkyl.

The term “unsubstituted” as used herein, refers to any compound that does not contain extra substituents attached to the compound. For example, an unsubstituted compound refers to the chemical makeup of the compound without added substituents (e.g., no non-hydrogen substituents). For example, unsubstituted proline is a proline amino acid even though the amino group of proline may be considered as disubstituted with alkyl groups.

The term “bond” as used herein in describing a substituent with atoms on both sides, refers to the absence of that substituent. For example, in the 4-atom sequence A-B-C-D, when B and C are both listed as being bonds, the result is the 2-atom sequence A-D. If only B is listed as being a bond, the result is the 3-atom sequence A-C-D.

The term “alkyl” as used herein, refers to any straight chain or branched, non-cyclic or cyclic, unsaturated or saturated aliphatic hydrocarbon containing from 1 to 10 carbon atoms, while the term “lower alkyl” has the same meaning as alkyl but contains from 1 to 3 carbon atoms. The term “higher alkyl” has the same meaning as alkyl but contains from 4 to 10 carbon atoms. Representative saturated straight chain alkyls include, but are not limited to, methyl, ethyl, n-propyl, n-butyl, n-pentyl, n-hexyl, n-heptyl, n-octyl, n-nonyl, and the like, while saturated branched alkyls include, but are not limited to, isopropyl, sec-butyl, isobutyl, tert-butyl, isopentyl, and the like. As used herein, a methyl substituent may be depicted as “CH₃” or “Me” or as a terminal bond with no indication of specific atoms.

The term “cycloalkyl” as used herein, refers to saturated and unsaturated cyclic alkyls. Representative saturated cyclic alkyls include, but are not limited to, C₃-C₁₄ (such as C₃-C₇) cycloalkyls, such as cyclopropyl, cyclobutyl, cyclopentyl, cyclohexyl, cycloheptyl, cyclooctyl, cyclododecyl, and the like; while unsaturated cyclic alkyls include, but are not limited to, cyclobutenyl, cyclopentenyl and cyclohexenyl, cyclohexadiene, and the like. Cyclic alkyls are also referred to herein as “homocycles” or “homocyclic rings”.

The term “bicyclic compounds” as used herein, encompasses “bridged” compounds, “fused” compounds and “spiro” compounds as described.

The term “spiro” or “spirocyclic” as used herein, refers to chemical structures having at least two rings sharing one common atom. The rings may be cycloalkyl, heterocyclyl or a combination thereof, and may include one or more aryl or heteroaryl rings. Examples include, but are not limited to, spirocyclic cyclopropanes, spirocyclic aziridines, spirocyclic cyclobutanes, spirocyclic azetidines, spirocyclic oxetanes, spirocyclic cyclopentanes, spirocyclic pyrrolidines, spirocyclic 1,3-dioxolanes, spirocyclic dioxanes, spirocyclic oxathiolanes, spirocyclic thiazolidines, spirocyclic cyclohexanes, spirocyclic piperidines and spirocyclic piperizines, where the other ring is cycloalkyl (e.g., cyclobutane, cyclopentane or cyclohexane) or heterocyclyl (e.g., piperidine, tetrahydropyran, tetrahydrofuran, azetidine or pyrrolidine). Exemplary embodiments include, but are not limited to, 1,4-dioxaspiro[4.5]decane, 1,4-dioxa-8-azaspiro[4.5]decane, 2-azaspiro[4.4]nonane, 2-azaspiro[4.4]nonane, 2,7-diazaspiro[4.4]nonane, 3-azaspiro[5.5]undecane, 3,9-diazaspiro[5.5]undecane, 6-azaspiro[3.4]octane, 6-azaspiro[2.5]octane, 1,3-dihydrospiro[indene-2,3′-pyrrolidine] and 3,4-dihydro-2H-spiro[naphthalene-1,4′-piperidine].

The term “bridged” as used herein, refers to a compound containing two nonadjacent atoms common to two rings. Exemplary embodiments include, but are not limited to, norbornane, bicyclo[1.1.1]pentane, bicyclo[2.2.1]heptane, 1,4-diazabicyclo[2.2.2]octane, 3,8-diazabicyclo[3.2.1]octane, 3-azabicyclo[3.2.1]octane, bicyclo[3.2.1]octane, 3,6-diazabicyclo[3.1.1]heptane, 3,6-diazabicyclo[2.2.1]heptane, and other bridged piperazines and bridged piperidines.

The term “fused” as used herein, refers to polycyclic ring systems in which any two adjacent rings have two, and only two, adjacent atoms in common (ortho-fused) and polycyclic ring systems in which a ring contains two, and only two, adjacent atoms in common with each of two or more rings of a contiguous series of ortho-fused rings (ortho- and peri-fused). An exemplary embodiment is pentalene and dibenzoxepine (ortho-fused) and pyrene (ortho- and peri-fused). Ortho-fused systems have “n” common sides and “2n” common atoms while peri-fused systems have “n” common sides and less than “2n” atoms in common. Other exemplary fused systems include, but are not limited to, fused cyclopropyl rings, fused aziridine rings, fused cyclobutane rings, fused azetidine rings, fused cyclopentane rings, fused pyrrolidine rings, fused cyclohexane rings, fused piperidine rings, fused tetrahydropyran rings and fused piperazine rings, where each of these rings may be fused to an identical or different ring, such a pyrrolidine ring fused to another pyrrolidine ring (e.g., octahydropyrrolo[3,4-c]pyrrole) or to a cyclohexane ring (e.g., octahydro-1H-indole or octahydro-1H-isoindole). Other examples include fused aryl or heteroaryl rings, such as a pyridine ring fused with a cycloalkyl ring (e.g., cyclopentane or cyclohexane) or with a heterocyclyl ring (e.g., tetrahydrofuran or tetrahydropyran).

The term “aromatic” or “aryl” as used herein, refers to any aromatic carbocyclic (i.e., all of the ring atoms are carbon) substituent such as, but not limited to, phenyl (from benzene), tolyl (from toluene), xylyl (from xylene) or multi-ring systems (e.g., naphthyl (from naphthalene) and anthracenyl (from anthracene).

The term “arylalkyl” or “aralkyl” as used herein, refers to any alkyl having at least one alkyl hydrogen atom replaced with an aryl moiety such as, but not limited to, benzyl, —(CH₂)₂phenyl, —(CH₂)₃phenyl, —CH(phenyl)₂, and the like.

The term “halogen” as used herein, refers to any fluoro, chloro, bromo, or iodo moiety.

The term “haloalkyl” as used herein, refers to any alkyl where at least one hydrogen atom (and including all hydrogen atoms) has been replaced with a halogen atom, such as, for example, trifluoromethyl, dichloromethyl, difluoromethyl, monofluoromethyl, monobromomethyl, 1,1,1-trifluoroethyl and the like.

The term “heteroaromatic” or “heteroaryl” as used herein, refers to any aromatic heterocyclic ring of 5 to 10 or more members and having at least one heteroatom selected from nitrogen, oxygen or sulfur, and containing at least 1 carbon atom, including, but not limited to, both mono- and bicyclic-ring systems. The heteroaryl ring may be attached as a substituent via a ring heteroatom or a carbon atom. Representative heteroaromatics include, but are not limited to, furan, benzofuran, thiophene, benzothiophene, pyrrole, indole, isoindole, 7-azaindole, 4-azaindole, 5-azaindole, 6-azaindole, 7-azaindazole, pyridine, quinoline, isoquinoline, oxazole, isoxazole, benzoxazole, pyrazole, imidazole, benzimidazole, thiazole, benzothiazole, isothiazole, 1,2,4-triazole, 1,2,3-triazole, tetrazole, 1,2,5-oxadiazole, 1,2,3-oxadiazole, 1,3,4-thiadiazole, pyridazine, pyrimidine, pyrazine, 1,2,4-triazine, 1,3,5-triazine, cinnoline, phthalazine, quinazoline, 1,8-naphthylpyridine, pyrido[3,2-d]pyrimidine, pyrido[4,3-d]pyrimidine, pyrido[3,4-b]pyrazine, pyrido[2,3-b]pyrazine, pteridine, triazolo-pyridines and the like.

The term “heteroarylalkyl” as used herein, means any alkyl having at least one alkyl hydrogen atom replaced with a heteroaryl moiety, such as —CH₂pyridinyl, —CH₂pyrimidinyl, and the like.

The term “heterocycle” or “heterocyclyl” or “heterocyclic ring” as used herein, refers to a nonaromatic ring which is either saturated or unsaturated and which contains 1 or more heteroatoms independently selected from nitrogen, oxygen, sulfur and silicon, wherein each of the nitrogen and sulfur heteroatoms may be in an oxidized state, and each of the nitrogen and silicon heteroatoms is substituted or unsubstituted and the nitrogen heteroatoms may be optionally quaternized, and includes bicyclic rings in which any of the above heterocycles are fused to an aryl or heteroaryl ring. The heterocyclic ring may be attached as a substituent via a ring heteroatom or a carbon atom. In various embodiments, heterocycles may contain 3 to 14 or more ring atoms (such as 3- to 7-membered monocyclic rings or 7- to 10-membered bicyclic rings) and include, but are not limited to, 2H-azirine, azetidine, 2,3-dihydroazete, 1,3-diazetidine, 2H-oxete, thietane, 2H-thiete, azetidin-2-one, morpholine, thiomorpholine, pyrrolidinone, pyrrolidinine, 2-pyrroline, 3-pyrroline, pyrazolidine, 2-pyrazoline, 2-imidazoline, imidazolidine, piperidine, piperazine, pyridin-2-ones (such as 2-pyridone and 1-methyl-2-pyridone), ethylene oxide (oxirane), ethylene imine (aziridine), ethylene sulfide (thiirane), oxetane, propylene oxide, 1,3-dioxolane, 1,2-oxathiolane, 1,3-oxathiolane, sulfolane, 2,4-thiazolidinedione, succinimide, 2-oxazolidone, dioxane, hydantoin, valerolactam, tetrahydrofuran, tetrahydropyran, 2H-pyran, 4H-pyran, thiane, 2H-thiopyran, 1,3-dithiane, 1,4-dithiane, 1,3,5-trithiane, pyrrolizidine, 1,4,5,6-tetrahydrocyclopenta[b]pyrrole, tetrahydropyridine, tetrahydropyrimidine, tetrahydrothiophene, tetrahydrothiopyran, indoline, isoindoline, decahydroisoquinoline, decahydroquinoline, 1,2,3,4-tetrahydroquinoline, 1,2-dihydroquinoline, 2H-benzo[e][1,3]oxazine, 2H-benzo[b][1,4]oxazine, quinolin-2(1H)-one, isoquinolin-1 (2H)-one, quinuclidine, 1-azaadamantane, 2-azaadamantane, 2,3-dihydroazepine, 2,5-dihydroazepine, oxepane, azonane, spiro[cyclobutane-1,3′-indole], 1-oxaspiro[4,5]decane, 1,6-dioxaspiro[3,4]octane, 2-oxa-7-azaspiro[3,5]nonane, 1,4-dioxa-7-azaspiro[4,4]nonane, 1,3-diazaspiro[4,4]non-2-en-4-one, 2,9-diazaspiro[5,5]undecan-1-one, 8-azaspiro[4,5]decane-7,9-dione, 1,4-dithia-7-azaspiro[4,4]nonane, and the like.

The term “heterocycloalkyl” as used herein, refers to any alkyl having at least one alkyl hydrogen atom replaced with a heterocycle, such as —CH₂morpholinyl, and the like.

The term “alkylamino” as used herein, means at least one alkyl moiety attached through a nitrogen bridge (i.e., —N-(alkyl)ₙ), where n=1 or 2, such as alkylamino or dialkylamino) including, but not limited to, methylamino, ethylamino, dimethylamino, diethylamino, and the like.

The term “alkyloxy” or “alkoxy”, as used herein, means any alkyl moiety attached through an oxygen bridge (i.e., —O-alkyl) such as, but not limited to, methoxy, ethoxy, and the like.

The term “thioalkyl” as used herein, means any alkyl moiety attached through a sulfur bridge (i.e., —S-alkyl) such as, but not limited to, methylthio, ethylthio, and the like.

The term “alkenyl” as used herein, refers to an unbranched or branched hydrocarbon chain having one or more carbon-carbon double bonds therein and may also be referred to as an “unsaturated alkyl”. The double bond of an alkenyl group can be unconjugated or conjugated to another unsaturated group. Suitable alkenyl groups include, but are not limited to vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl group can be unsubstituted or substituted with one or two suitable substituents.

The term “alkynyl” as used herein, refers to unbranched or branched hydrocarbon chain having one or more carbon-carbon triple bonds therein and may also be referred to as an “unsaturated alkyl”. The triple bond of an alkynyl group can be unconjugated or conjugated to another unsaturated group. Suitable alkynyl groups include, but are not limited to ethynyl, propynyl, butynyl, pentynyl, hexynyl, methylpropynyl, 4-methyl-1-butynyl, 4-propyl-2-pentynyl-, and 4-butyl-2-hexynyl. An alkynyl group can be unsubstituted or substituted with one or two suitable substituents.

As used herein, “reactive groups” refer to nucleophiles, electrophiles, or radically active groups, i.e., groups that react in the presence of radicals. A nucleophile is a moiety that forms a chemical bond to its reaction partner (the electrophile) by donating both bonding electrons. Electrophiles accept these electrons. Nucleophiles may take part in nucleophilic substitution, whereby a nucleophile becomes attracted to a full or partial positive charge on an element and displaces the group it is bonded to. Alternatively, nucleophiles may take part in substitution of carbonyl group. Carboxylic acids are often made electrophilic by creating succinyl esters and reacting these esters with aminoalkyls to form amides. Other common nucleophilic groups are thiolalkyls, hydroxylalkyls, primary and secondary amines, and carbon nucleophiles such as enols and alkyl metal complexes. Other preferred methods of ligating proteins, oligosaccharides and cells using reactive groups are disclosed (Lemieux et al., Trends in Biotechnology 1998, 16, 506, incorporated herein by reference in its entirety). In yet another preferred method, one provides reactive groups for the Staudinger ligation, i.e., “click chemistry” with an azide comprising moiety and alkynyl reactive groups to form triazoles. Michael additions of a carbon nucleophile enolate with an electrophilic carbonyl, or the Schiff base formation of a nucleophilic primary or secondary amine with an aldehyde or ketone may also be utilized. Other methods of bioconjugation are provided (Hang et al. Accounts of Chemical Research 2001, 34, 727, and Kiick et al. Proc Natl Acad Sci U.S.A. 2002, 99, 19, both of which are incorporated by reference in its entirety).

The term “biocompatible” as used herein, refers to any material that does not illicit a substantial detrimental response in the host. There is always concern when a foreign object is introduced into a living body that the object will induce an immune reaction, such as an inflammatory response that will have negative effects on the host. In the context of this invention, biocompatibility is evaluated according to the application for which it was designed: for example, a bandage is regarded as biocompatible with the skin, whereas an implanted medical device is regarded as biocompatible with the internal tissues of the body. Preferably, biocompatible materials include, but are not limited to, biodegradable and biostable materials. A substantial detrimental response has not occurred if an implant comprising the material is in close association to its implant site within the host animal and the response is better than a tissue response recognized and established as suitable from materials provided in an ASTM. ASTM subcommittee F04.16 on Biocompatibility Test Methods has developed biocompatibility standards for medical and surgical materials and devices which includes E1262-88, F612-20, F719-20e1, F720-17, F748-16, F749-20, F750-20, F756-17; F763-04, F813-20, F895-11, F981-04, F1027-86, F1408-20a, F1439-03, F1877-16, F1903-18, F1904-14, F1983-14, F1984-99, F2147-01, F2148-18, F2382-18, F2808-17, F1288-19 and F2909-19, each of which is incorporated herein by reference. For example, materials that are to be used in contact with the blood stream must be composed of materials that meet hemocompatibility standards. One of these tests is for damage to red blood cells, which can result in hemolysis that is, rupturing of the cells, as described in F756-17 Standard Practice for Assessment of Hemolytic Properties of Materials.

As used herein, a “bioactive substance” refers to any of a variety of chemical moieties and that binds with a biomolecule such as, but not limited to, peptides, proteins, enzymes, receptors, substrates, lipids, antibodies, antigens, and nucleic acids. In certain preferred embodiments, the bioactive substance is a biomolecule but it is not intended that the bioactive substance be limited to biomolecules. In other preferred embodiments, the bioactive substances provide hydrophobic, hydrophilic, or electrostatic interactions, such as polycarboxylic acids that are anionic at physiological pH. In other preferred embodiment, the alkaline growth factors (with isoelectric point above 7) are retained via favorable electrostatic interactions by the polycarboxylates, and subsequently released in a controlled and sustained manner.

“Cancer” is a term used for a physiological condition in mammals that is typically characterized by unregulated cell growth. Examples of cancer include, but are not limited to, carcinoma, lymphoma, leukemia, blastoma, and sarcoma. More particular examples of such cancers include squamous cell carcinoma, small cell lung cancer, non-small cell lung cancer (NSCLC), glioma, Hodgkin's lymphoma, non-Hodgkin's lymphoma, acute myeloid leukemia (AML), multiple myeloma, gastrointestinal cancer, renal cell carcinoma, renal cancer (e.g., advanced renal cell carcinoma), ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, urothelial carcinoma (including local advanced or metastatic urothelial carcinoma), bladder cancer, hepatoma, breast cancer and head and neck cancer.

The term “stereoisomer” refers to compounds that have the same atomic connectivity but different atomic arrangement in space. Stereoisomers include cis-trans isomers, E and Z isomers, enantiomers, diastereomers and atropisomers. In the context of the present invention, the term “enantiomerically pure” is understood to mean that the compound in question with respect to the absolute configuration of the chiral center is present in an enantiomeric excess of more than 95%, preferably more than 97%.

The present disclosure contemplates all such compounds, including cis and trans isomers, (−)- and (+)-enantiomers, (R)- and (S)-enantiomers, diastereomers isomers, (D)-isomers, (L)-isomers, atropisomers, tautomers and racemic and other mixtures thereof, such as enantiomers or diastereomeric enriched mixtures, all of which are within the scope of the present disclosure. Insofar as compounds of the invention as defined herein may exist in optically active or racemic forms by virtue of one or more asymmetric carbon atoms, the invention includes in its definition any such optically active or racemic form. The synthesis of optically active compounds may be carried out by standard techniques of organic chemistry well known in the art such as, for example, by synthesis from optically active starting materials or by resolution of a racemic compound. Similarly, the enantiomeric or diastereomeric purity of a compound may be evaluated using standard laboratory techniques.

The pharmaceutical compositions of the invention can take any suitable form for the desired route of administration. Where the composition is to be administered orally, any suitable orally deliverable dosage form can be used, including without limitation water, glycols, oils, alcohols, and the like in the case of oral liquid preparations such as suspensions, syrups, elixirs, emulsions, and solutions; or solid carriers such as starches, sugars, kaolin, diluents, lubricants, binders, disintegrating agents, and the like in the case of powders, pills, capsules, and tablets. Because of their ease in administration, tablets and capsules represent the most advantageous oral dosage unit forms. Injectable compositions or intravenous infusions are also provided in the form of solutions, suspensions, and emulsions. For parenteral compositions, the carrier usually comprises sterile water and possibly other ingredients to aid solubility. Injectable solutions may be prepared in which the carrier comprises a saline solution, a glucose solution, or a mixture of a saline and a glucose solution. Suitable oils include, for example, peanut oil, sesame oil, cottonseed oil, corn oil, soybean oil, synthetic glycerol esters of long chain fatty acids, and mixtures of these and other oils. In compositions suitable for percutaneous administration, the carrier optionally comprises a penetration enhancing agent and/or a suitable wetting agent, optionally combined with suitable additives as needed, where the additives may facilitate administration of the composition to the skin and/or may facilitate preparation of the compositions to be delivered. These compositions may be administered in various ways, e.g., as a transdermal patch or as an ointment. Acid or base addition salts of the compounds of the invention are typically more suitable in the preparation of aqueous compositions due to their increased water solubility over the corresponding neutral form of the compounds.

The pharmaceutical compositions of the invention may comprise one or more of a filler, diluent, adjuvant, vehicle, or other excipient to facilitate storage and/or administration of the active ingredients contained therein.

In an exemplary embodiment, a pharmaceutical composition according to the present invention may contain one or more additional therapeutic agents, for example, to increase efficacy or to decrease undesired side effects. In a particular embodiment, the pharmaceutical composition further contains one or more additional therapeutic agents useful to treat or inhibit a disease mediated directly or indirectly by PI3K. Examples of such agents include, without limitation, agents to treat or inhibit cancer, Huntington's disease, cystic fibrosis, liver fibrosis, renal fibrosis, pulmonary fibrosis, skin fibrosis, rheumatoid arthritis, diabetes, or heart failure.

In a specific embodiment, the additional therapeutic agent to be included is an anti-cancer agent. Examples of an anti-cancer agent include, but are not limited to, DNA-damaging cytotoxic drugs, alkylating agents such as cyclophosphamide, dacarbazine, and cisplatin; anti-metabolites such as methotrexate, mercaptopurine, thioguanine, fluorouracil, and cytarabine; plant alkaloids such as vinblastine and paclitaxel; antitumor antibiotics such as doxorubicin, bleomycin and mitomycin; hormones/antihormones such as prednisone, tamoxifen, and flutamide; other types of anticancer agents such as asparaginase, rituximab, trastuzumab, imatinib, retinoic acid, and derivatives, colony stimulating factors, amifostine, camptothecin, topotecan, thalidomide analogs such as lenalidomide, and proteasome inhibitors such as Velcade.

In another embodiment, the present invention provides a method of inhibiting or treating diseases arising from abnormal cell proliferation and/or differentiation in a subject in need thereof, comprising administering to the subject a therapeutically effective amount of one or more compounds according to the present invention. In one embodiment, the method of inhibiting or treating disease comprises administering to a subject in need thereof, a composition comprising an effective amount of one or more compounds of the invention and a pharmaceutically acceptable carrier. The composition to be administered may further contain a therapeutic agent such as an anti-cancer agent.

The compounds of the invention are defined herein by their chemical structures and/or chemical names and are generally listed according to the IUPAC or CAS nomenclature system. Abbreviations that are well known to one of ordinary skill in the art may be used. When a compound is referred to by both a chemical structure and a chemical name, and the chemical structure and chemical name conflict, the chemical structure is intended to be determinative of the compound's identity.

The present invention includes compounds labeled with various radioactive or nonradioactive isotopes. Examples of atomic isotopes may include, but are not limited to, deuterium (²H), tritium (³H), iodine-125 (¹²⁵I), carbon-14 (¹⁴C), nitrogen-15 (¹⁵N), sulfur-35 (³⁵S) and chlorine-36 (³⁶Cl). In an exemplary embodiment, one or more hydrogen atoms in a compound of the invention can be replaced by deuterium. In various embodiments, a compound of the invention includes at least one deuterium atom, or two or more deuterium atoms, or three or more deuterium atoms, etc. As described herein, compounds of the invention may also be radiolabeled with a radioactive isotope such as tritium (³H), iodine-125 (¹²⁵I), and carbon-14 (¹⁴C). A radiolabeled compound is useful as a therapeutic or prophylactic agent, provides a reagent for research such as for an assay, and/or provides a diagnostic agent for techniques such as in vivo imaging. Synthetic methods for incorporating isotopes into organic compounds are well known in the art.

In an embodiment of the invention, a compound of the invention as defined herein (such as a compound of any one of Formula (1), (2), (3), (4), (5) or (6)) or a pharmaceutically-acceptable salt thereof, exists as a single enantiomer being in an enantiomeric excess (% ee) of ≥95%, such as ≥98%, such as ≥99%.

In an embodiment of the invention, a pharmaceutical composition comprises a compound of the invention as defined herein (such as a compound of Formula I) or a pharmaceutically-acceptable salt thereof, where the compound exists as a single enantiomer being in an enantiomeric excess (% ee) of ≥95%, such as ≥98%, such as ≥99%.

In an exemplary embodiment of the invention, the disease or disorder to be treated by the compounds of the invention is selected from congenital lipomatous overgrowth, vascular malformations, epidermal naevi, scoliosis/skeletal and spinal syndrome (CLOVES), mosaic tissue overgrowth syndromes, venous malformations and brain malformations associated with severe epilepsy or PIK3CA-related overgrowth syndrome (PROS) (Keppler-Noreuil et al., Am J Med Genet A. 2015, 167A, 287; Kurek et al. Am. J. Hum. Genet. 2012, 90, 1108).

In an exemplary embodiment of the invention, the cancer to be treated is a cancer bearing a PI3K H1047 mutation (such as H1047R) (Thorpe et al., Nat Rev Cancer 2015, 15, 7).

The compounds of the invention (such as defined by Formula (1) through Formula (6)) are typically PI3Kα H1047R mutant-selective inhibitors that exhibit greater selectivity for the H1047R mutation over the wild-type. As such, the compounds may decrease the amount of phosphorylated AKT (pAKT) and decrease proliferation selectively in PI3Kα H1047R mutant cell lines, preferably across several tumor types.

A PI3K H1047R mutant selective inhibitor of the invention (such as defined by Formula (1) through Formula (6)) dosed in combination with a selective estrogen receptor degrader (SERD) such as, but not limited to, fulvestrant, elacestrant, camizestrant or vepdegestrant may exhibit a combination benefit leading to tumor regression in ER+/PI3K H1047R mutant tumors such as, but not limited to, the breast cancer xenograft model T47D, at doses where little or no regression would be observed with either single agent.

A PI3K H1047R mutant selective inhibitor of the invention (such as defined by Formula (1) through Formula (6)) dosed in combination with a HER2 inhibitor such as, but not limited to, tucatinib or trastuzumab may exhibit a combination benefit leading to tumor regression in ER−/HER2+/PI3K H1047R mutant tumors such as, but not limited to, the breast cancer xenograft model HCC1954, at doses where little or no regression would be observed with either single agent.

Compounds of Formula (1) of the present invention may be generally prepared according to the synthetic routes identified in Schemes 1-11.

In Scheme 1, synthesis may begin with an appropriately substituted 2,3-dihydro-1H-inden-1-one. In the case of 1 where R₇ is methyl, this is commercially available. In other cases, the starting material may be prepared via established methods known to those skilled in the art. Nitrosation of indenones 1 to convert to the oxime derivatives 2 may be accomplished using established methods (for examples see Touster, O.; Org. Reactions, VII, 1953, 327). A Beckmann type rearrangement mediated by phosphorus pentachloride can convert oximes 2 into chloroisoquinolones 3 (Cushman, M.; Dekow, F. W. Tetrahedron 1978, 34 (10), 1435-9). Alkylation of isoquinolone intermediates 3 with an appropriate electrophile and base can give nitrogen-substituted isoquinolones 4. In the case of where R₆ is methyl this may be accomplished with methyl iodide and an appropriate base (such as sodium hydride). Other electrophiles and alkylating agents, which would be known to those skilled in the art, may also be employed. In order to convert the bromide of isoquinolones 4 to methyl ketones 5, a Stille coupling reaction may be employed using an appropriate tin reagent, such as (a-ethoxyvinyl)tributyl tin, followed by acid hydrolysis (Sugiyama, et al., Bull. Chem. Soc. Jpn. 1987, 60 (2), 767-768). Alternatively, conversion of 4 to 5 may be accomplished by other established methods (for example, using a Heck coupling reaction with an appropriate enol-ether followed by acid hydrolysis (Mingcui, L. et al., Org. Biomol. Chem., 2010, 8, 2012-2015). Reduction of ketones 5 to secondary alcohols 6 may then be accomplished by use of an appropriate hydride reducing agent (such as sodium borohydride). It should be understood that apart from the methodology described in Scheme 1, there are other reported methods that can be utilized to prepare isoquinolones such as 3 or 4 and their derivatives (for examples, see Li, B. et al., Tetrahedron Letters 2010, 51 (29), 3748-3751, and Wang, R.; et al., Organic & Biomolecular Chemistry 2011, 9 (16), 5802-5808).

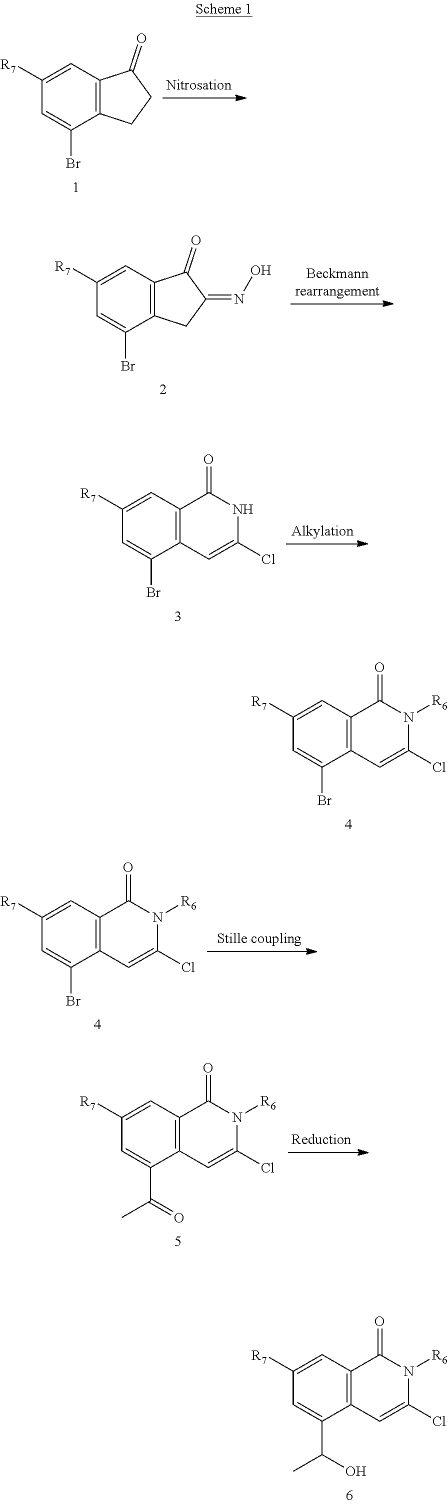

In Scheme 2, alcohols 6 can be converted to anthranilic acid derivatives 8 via a number of different methodologies. The alcohol functionality may first be converted to a leaving group such as a bromide or a mesylate utilizing commonly known methods. Nucleophilic displacement with an anthranilic acid ester (methyl, t-butyl or other commonly employed esters may be used) on 7 gives compounds 8. An alternative is to use a Mitsunobu type of reaction of an anthranilic acid ester 9 directly with an alcohol 6 to give 8 directly. In some cases, the transient use of an activating group (such as a 2,4-dinitrobenzenesulfonyl group) on the anthranilic acid amine functionality can facilitate the Mitsunobu reaction. The use of 2,3-dichloro-5,6-dicyanobenzoquinone (DDQ) and triphenylphosphine may also be employed in the direct reaction of an alcohol 6 with an anthranilic acid ester (Shalit, T.; et al., Tetrahedron Letters 2010, 51, 5988-5991; Iranpoor, N.; et al., Tetrahedron 2009, 65, 3893-3899; Panday, S. K., Mini-Reviews in Organic Chemistry 2019, 16 (2), 127-140; Fukuyama, Tohru; et al., Tetrahedron Letters 1997, 38 (33), 5831-5834).

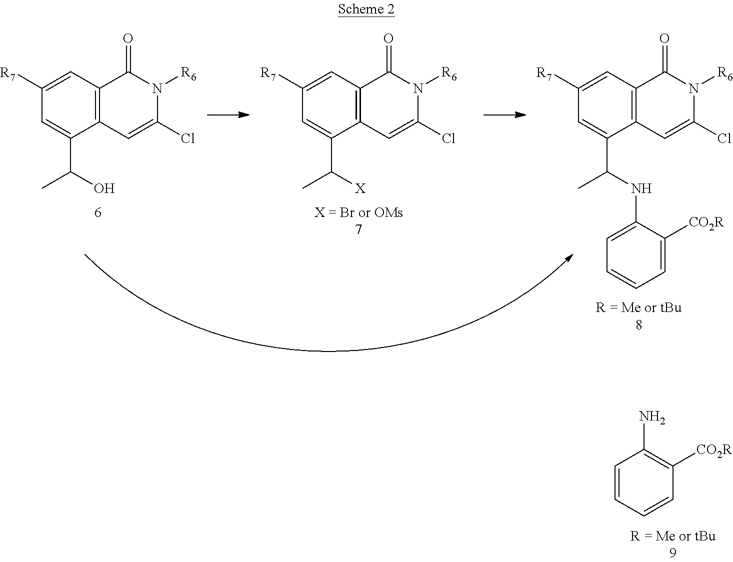

An alternate mode of synthesis of a single enantiomer intermediate analogous to 8 is depicted in Scheme 3. This reaction sequence utilizes the formation of a chiral sulfinyl imine to control the stereochemistry. Such methods have been extensively reported. Ketones 5 may be converted to chiral sulfinyl-imines 10 via known procedures, which may then in turn be reduced to sulfinyl-amines 11 in a stereo-controlled fashion using a suitable reducing agent (Datta and Ellman, J. Org. Chem. 2010, 75, 6283-6285; Ellman et al., Acc. Chem. Res. 2002, 35, 984-995; Ellman et al., J. Org. Chem. 2007, 72, 626-629; Colyer et al., Journal of Organic Chemistry 2006, 71 (18), 6859-6862). With use of the R isomer of the sulfinyl group, generally, resulting a predominantly the R, R-isomer of the product when (for example) the reducing agent used is a mixture of sodium borohydride and cerium chloride-heptahydrate. The use of this particular reducing system has been shown to be effective at reducing imines and may often give enhanced stereo-control in similar reductions (Hua et al, Synthesis 1991, (11), 970-4; Zhu et al, Journal of Chemical Research 2015, 39 (7), 390-393). The major isomer may be separated from the other minor isomer via standard chromatographic means. As has been demonstrated in the preceding literature references a judicious choice of the antipode of the sulfinyl-imine and the reducing agent may give access to either antipode of the sulfinyl-amine. The sulfinyl-amines can be cleaved to the single enantiomer of the chiral amine 12 using standard conditions (such as hydrogen chloride in dioxane). A standard coupling reaction of amines 12 with aryl iodides 13 (such as an Ullman coupling or Buchwald-Hartwig coupling) may then give anthranilic acid derivatives 14 (Yang et al., Organic Process Research & Development 2022, 26 (6), 1690-1750; Surry and Buchwald, Chemical Science 2011, 2 (1), 27-50).

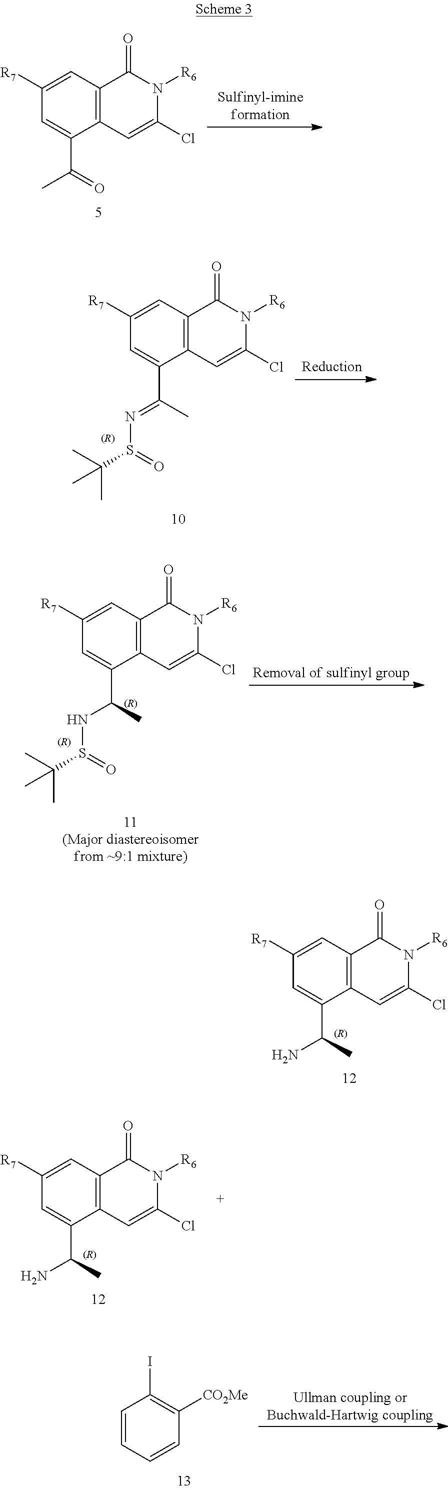

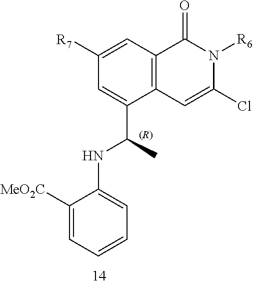

Certain final compounds may be prepared as outlined in Scheme 4. Intermediates 8 may be converted to bromide or iodide 15. Then, 8 or 15 may be reacted under suitable coupling conditions with an amine, wherein R^{I} and R^{II} may be either alkyl or aryl, or one of R^{I} and R^{II} may be hydrogen. R^{I} and R^{II} may also be joined to form a ring. In some cases, the R^{I} and/or R^{II} groups may be further elaborated prior to subsequent steps. Also, halogen-substituted isoquinolones 8 or 15 could be reacted under suitable coupling conditions with alkyl or aromatic boronates or boronic acids, or alkyl carboxylic acids, or alkynes to give carbon-linked versions of 16. Standard ester hydrolysis conditions may then be employed to convert esters 16 to carboxylic acids 17. The resulting product may then be separated into its individual enantiomers (18 and 19) utilizing chiral chromatography (HPLC or SFC).

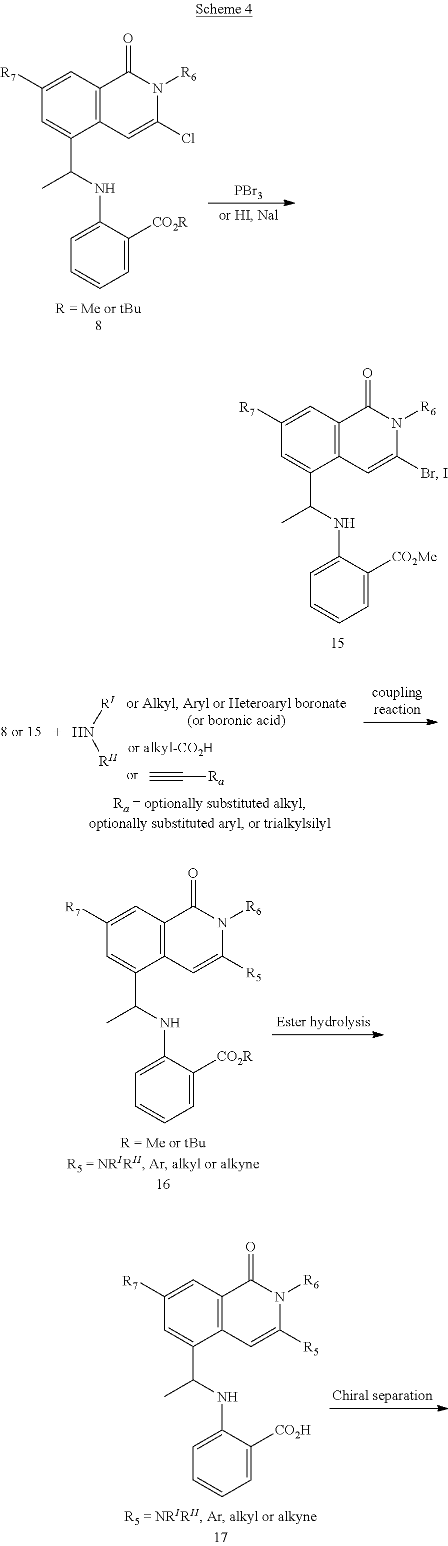

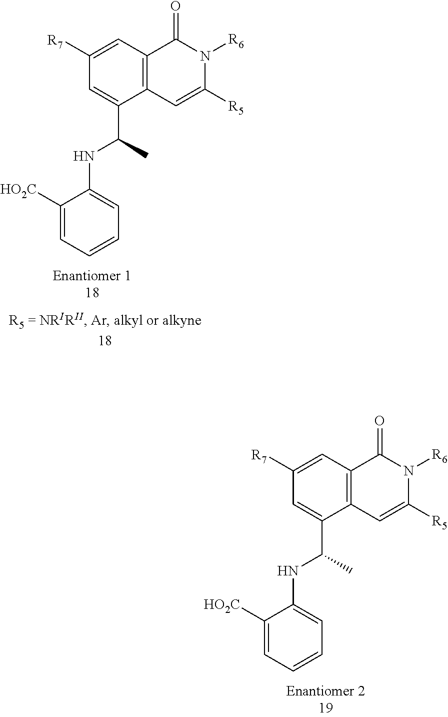

The single enantiomer of isoquinolines 21 may be prepared directly as shown in Scheme 5 from the enantiomerically pure intermediates 20 using chemistry analogous to that shown in Scheme 4.

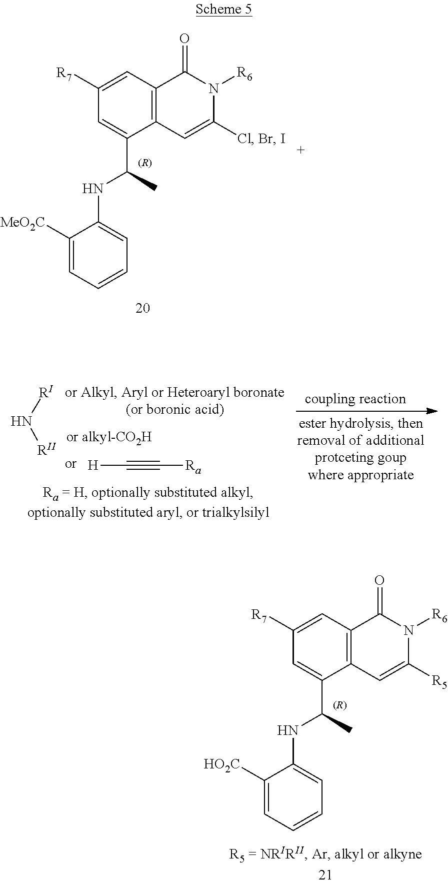

In some cases, the order of reactions may be adjusted as shown in Scheme 6. In this case Buchwald-Hartwig couplings with intermediates 10 and appropriately substituted amines may give amine-substituted isoquinolones 21. Removal of the sulfinyl group from compounds 21 to give amines 22 may then be followed by a Buchwald-Hartwig coupling with (for example) an ester of 2-iodobenzoic acid (or another appropriately substituted benzoic acid derivative). Ester hydrolysis of that product may then give benzoic acids such as 23. Alternatively intermediate 22 may undergo an S_{N}Ar reaction with electrophile-containing heterocycles (for example, methyl 6-chloro-3-fluoropyridine-2-carboxylate 24 or other suitably substituted heterocycles). A subsequent ester hydrolysis step may then give carboxylic acid compounds such as 25.

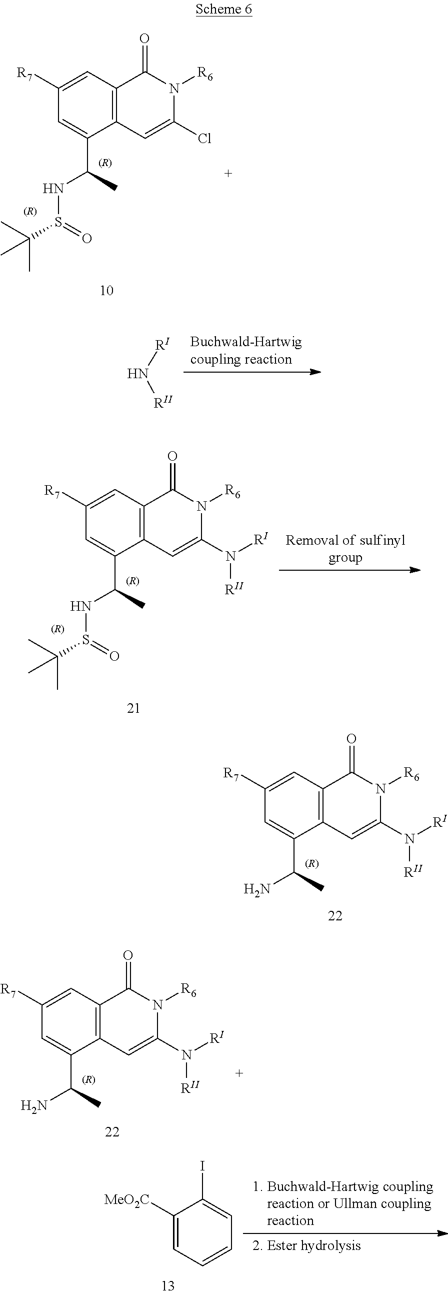

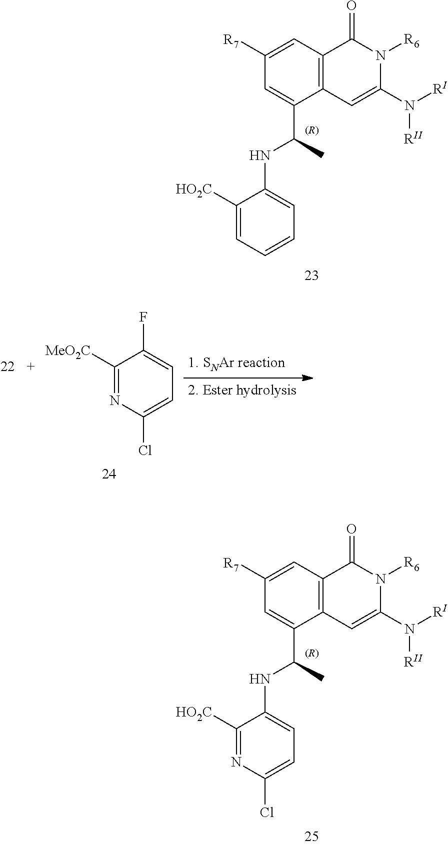

Compounds where there is an oxygen- or sulfur-linked substitution from the 3-position of the isoquinolone ring may be prepared as shown in Scheme 7. Intermediates 14 may undergo Buchwald-Hartwig couplings with appropriate thiols 26 compound to give thioethers 27. Ester hydrolysis may then give carboxylic acid-containing compounds such as 28. Alternatively, intermediates 14 may undergo S_{N}Ar reactions with appropriate alcohols 29. This reaction may be mediated by a base (for example, sodium hydride). The resulting ethers 30 may then be subjected to ester hydrolysis conditions to give compounds such as 31.

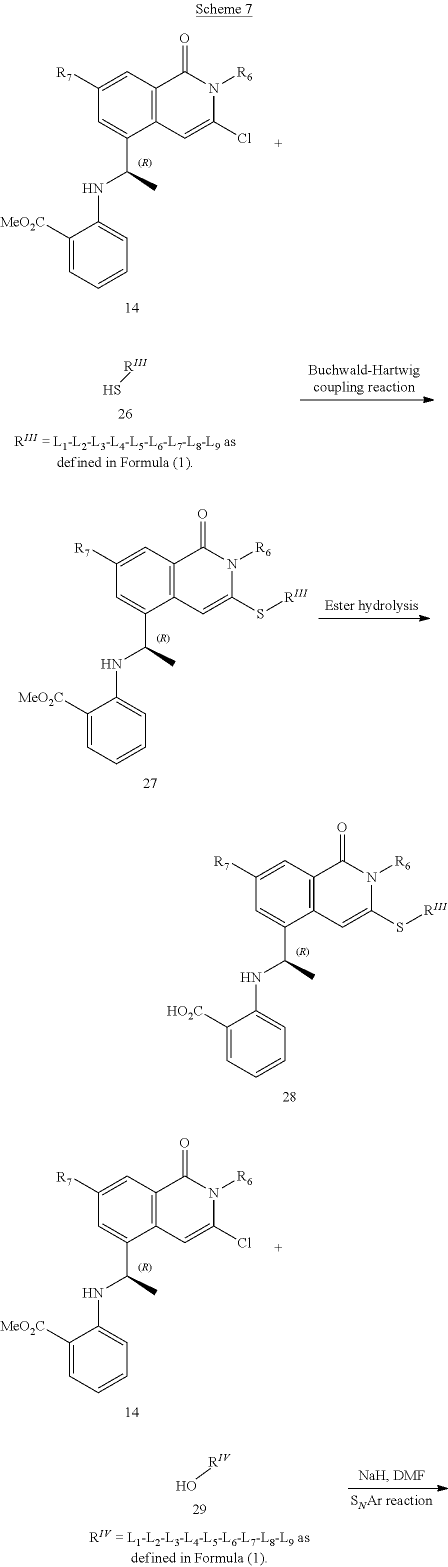

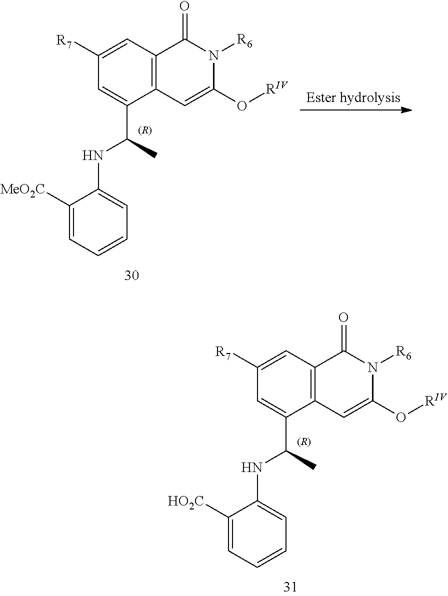

In the cases where the substitution from the 3-position of the isoquinolone ring is either an alkyl or an alkenyl group, these compounds may be prepared as shown in Scheme 8. Intermediates 8 may be transformed via a Suzuki coupling reaction (Stanforth, S. P. Tetrahedron 1998, 54(3/4), 263-303) with suitable alkenyl-boronates (or boronic acids)₃₂ with subsequent removal of the ester to give carboxylic acids 33. The racemate 33 may then be separated by chiral chromatography methods to give the individual enantiomers 34 and 35. Alternatively, the double bond of 33 may be reduced under standard hydrogenation conditions (e.g., hydrogen with a palladium catalyst). Following chiral chromatographic separation this may give enantiomers 36 and 37. In the case where R^{V} and R^{VI} do not form a symmetrical arrangement, further isomers may result which may also be separated chromatographically.

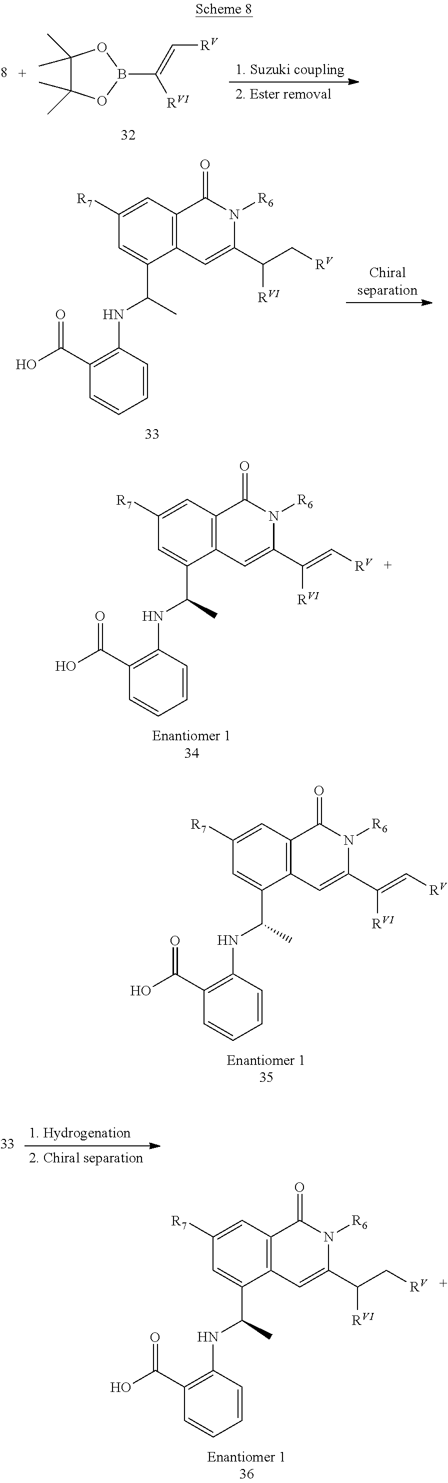

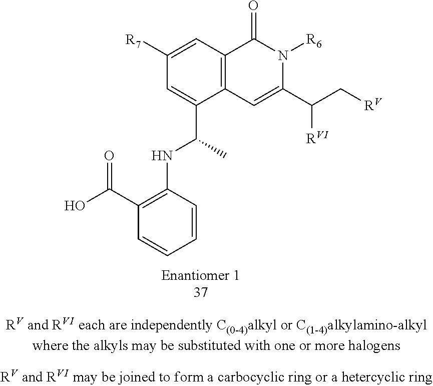

2-Fluoro isoquinolones may be prepared as shown in Scheme 9. Isoquinolone intermediate 38 may undergo reactions with appropriate electrophilic fluorinating reagents, such as Selectfluor™ (1-chloromethyl-4-fluoro-1,4-diazoniabicyclo[2.2.2]octane bis(tetrafluoroborate), followed by hydrolysis to give 2-fluoroisoquinolones 39.

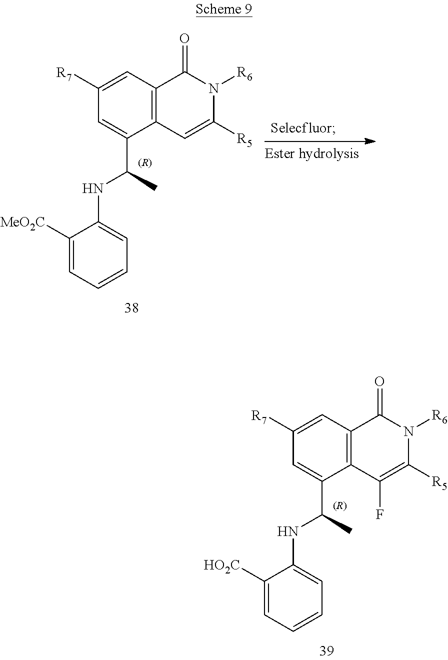

An alternative mode of synthesizing carboxylic acid-containing isoquinolones is depicted in Scheme 10. A Suzuki coupling reaction between chloro-containing intermediate 10 and an appropriate aryl or heteroaryl boronate (or boronic acid) followed by removal of the sulfinyl group may give aryl-substituted isoquinolone amines 40. An Ullman or Buchwald-Hartwig coupling between amines 40 and halogen-substituted benzoates (for example, the methyl ester of 2-iodobenzoic acid or another suitably substituted aryl halide), followed by ester hydrolysis may give carboxylic acid compounds such as 41. Alternatively, intermediates 40 may undergo an S_{N}Ar reaction with halogen-substituted heteroaryl esters (for example, methyl 6-chloro-3-fluoropyridine-2-carboxylate or another suitably substituted heterocycle) to give, following ester hydrolysis, carboxylic acid compounds such as 42.

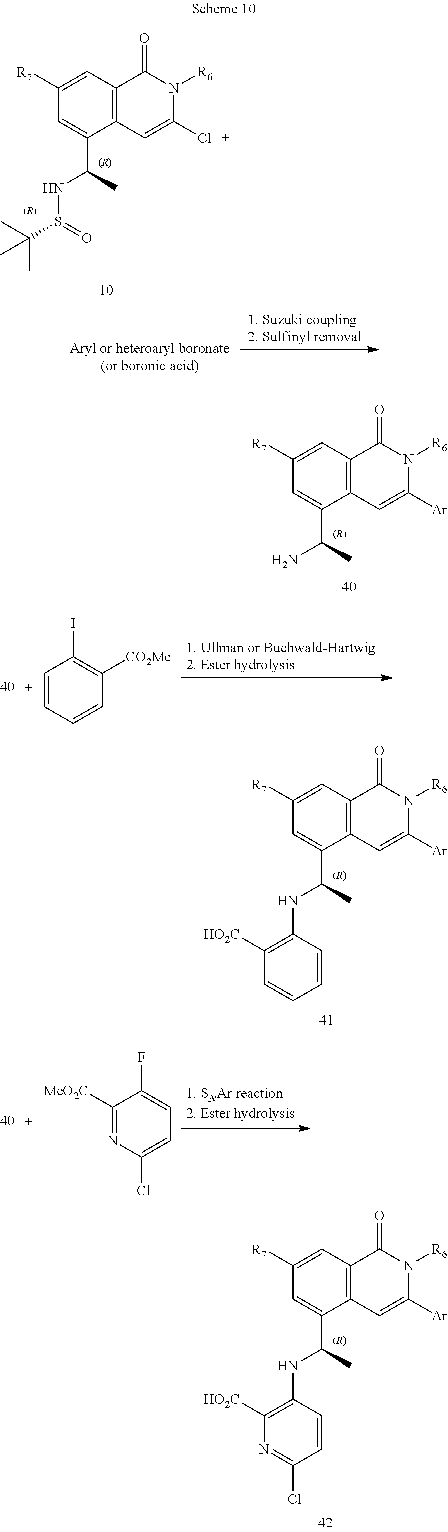

An additional mode of synthesizing carboxylic acid-containing isoquinolones is depicted in Scheme 11. Chloro intermediates 14 may be converted to boronates 43 under appropriate conditions, such as by treatment with PdCl₂(dppf) and bis(pinacolato)diboron. Boronates 43 may then be reacted with aryl or heteroaryl halides followed by hydrolysis to give functionalized isoquinolones 44.

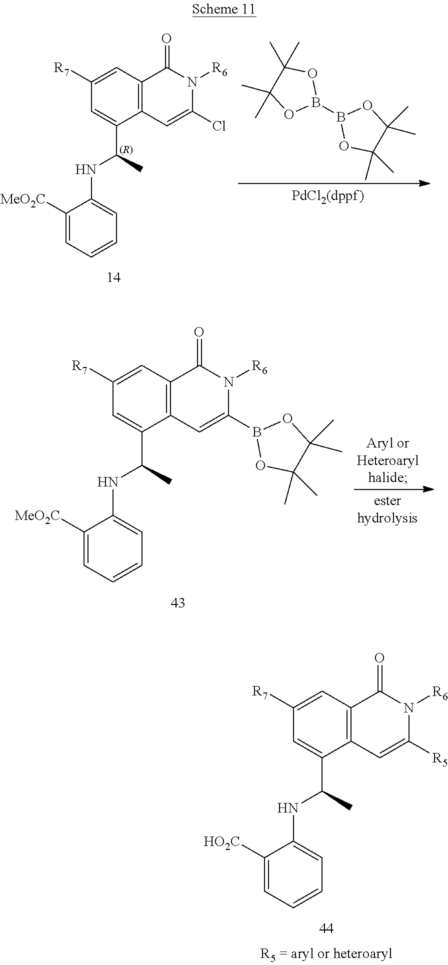

The chemistry depicted in Schemes 1-11 gives various modes of synthesis of the compounds described. It should be understood that other variations on these modes may be employed and that the precise protecting groups, order of reactions or particular type of transition metal catalyzed coupling reaction may be chosen as required and would be known to those skilled in the art.

The following compounds of Formula (4) represent various embodiments of the present invention where R₁′ and R₅ have been varied in a structure where all other atoms have been set. The carbon atom marked with * is a chiral center and exists as a (R)- and (S)-racemic mixture or as either (R)- or (S)-enantiomer. The listing of substituents within brackets (in Formula (4) or for a listed embodiment of R₅ or R₁′) indicates individual compounds containing one of each of the substituents. Where present in embodiments of R₅ or R₁′, each X is independently N or CH; each X₂ is independently O or CH₂; each X, is independently O, CH₂ or NₖNₘ; each Rₕ and each Rᵢ are independently selected from H, CH₃, c-Pr, c-Bu, CF₃ and OH; each Rⱼ is independently selected from CF₃, CH₂CF₃, CH₂CF₂H, OCH₃, OCF₃, OCH₂CF₃, Oc-Pr, aryl, heteroaryl, COCH₃ and CO₂CH₃; each Rₖ and Rₘ is CH₃, CH₂CH₃, CH₂CH₂CH₃, CH₂CH₂OH, CH₂CH₂N(CH₃)₂, COCH₃; and each “A” is selected from O, S, S(O) and S(O)₂. All chiral centers in the below R₅ structures that are not specified, exist as a (R)- and (S)-racemic mixture or as either (R)- or (S)-enantiomer.

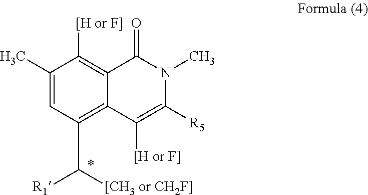

where R₅ is selected from:

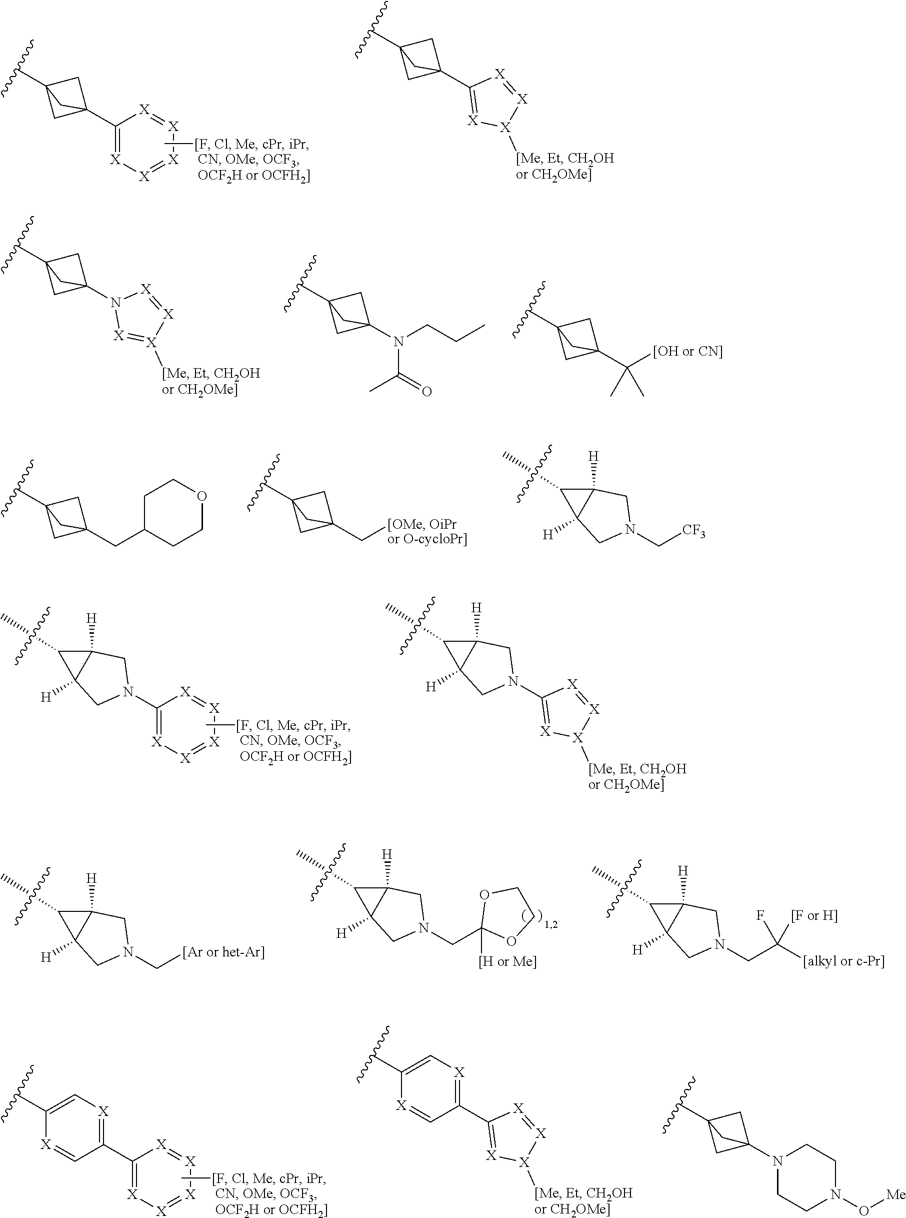

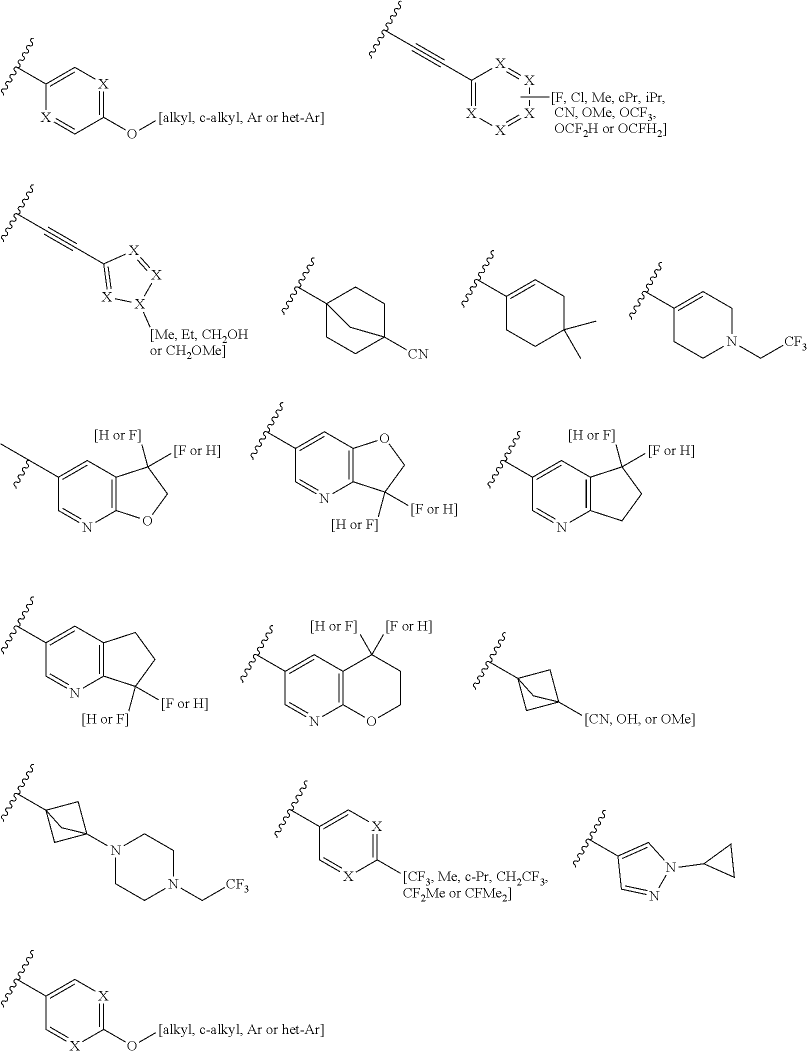

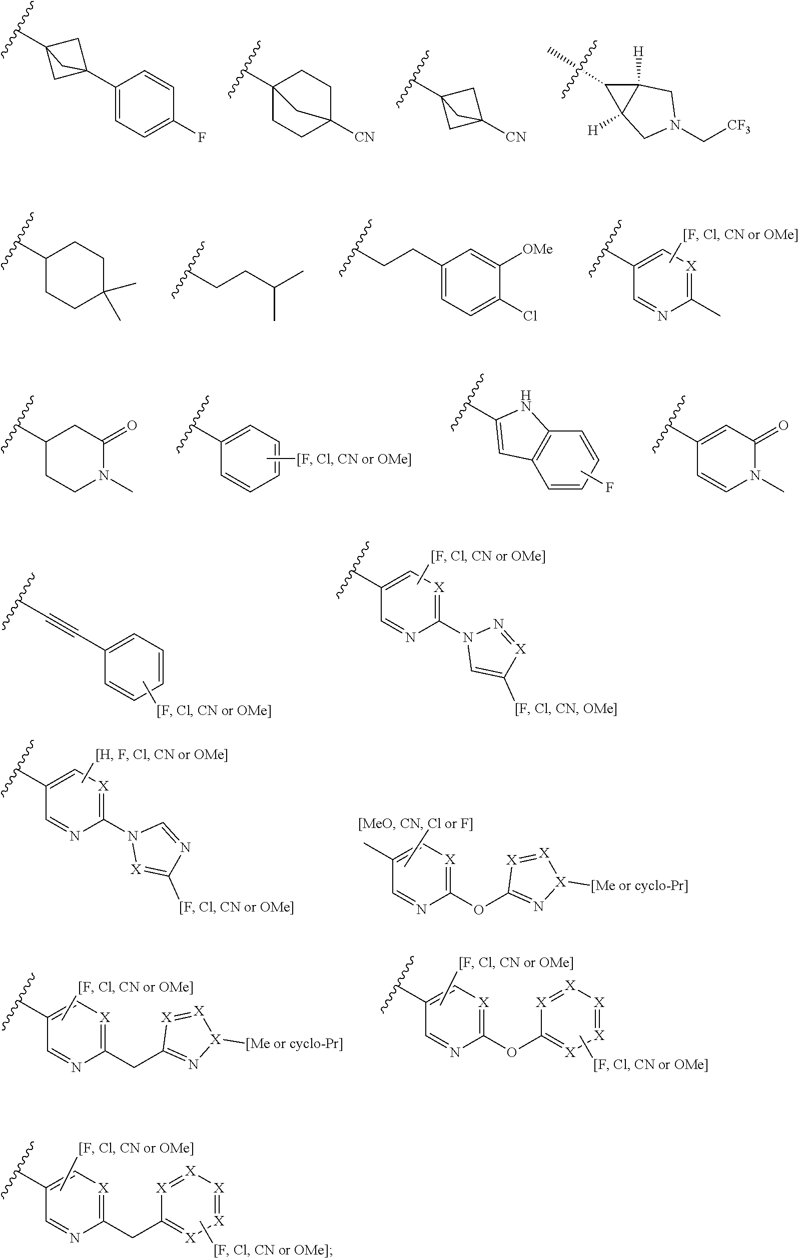

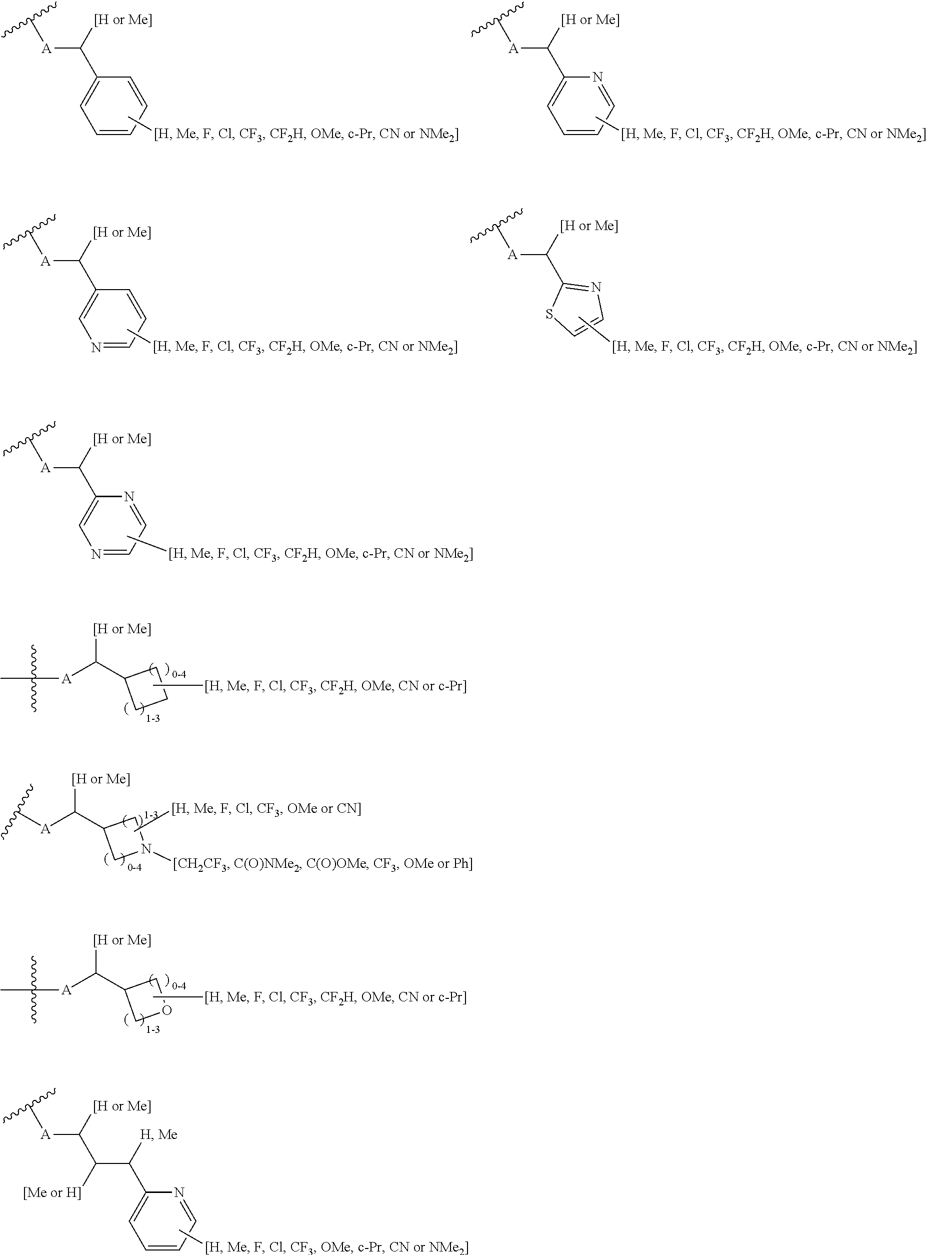

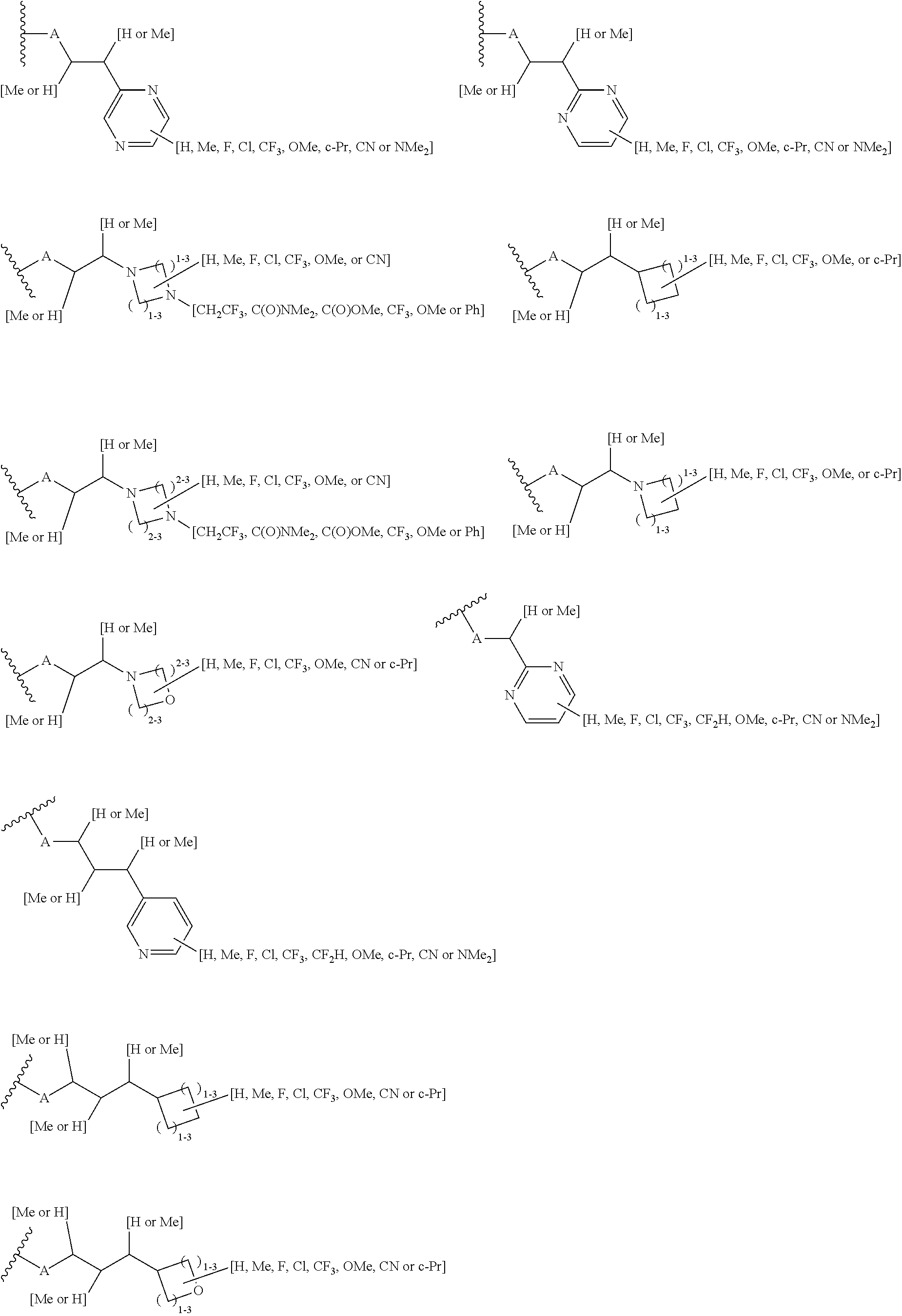

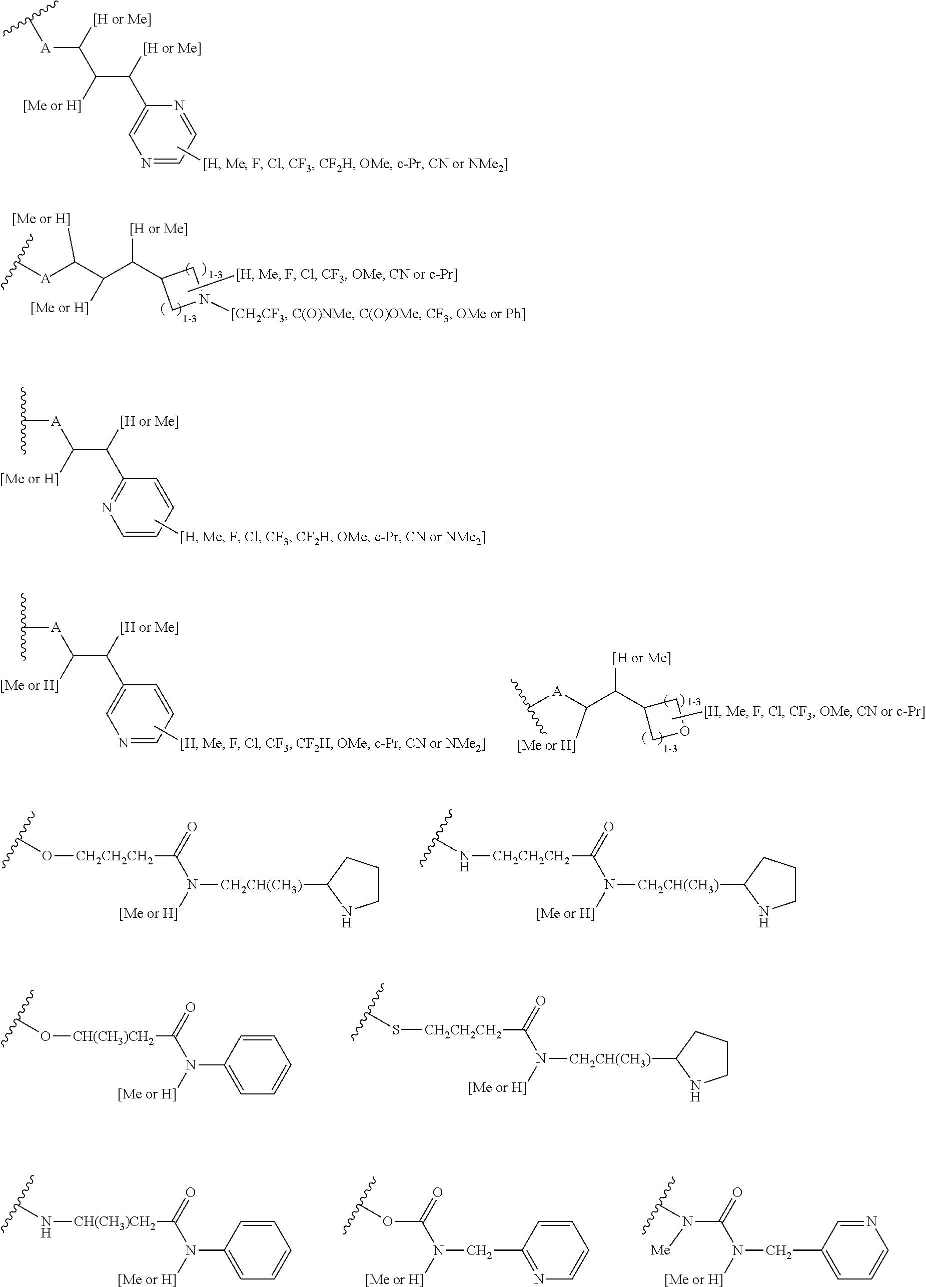

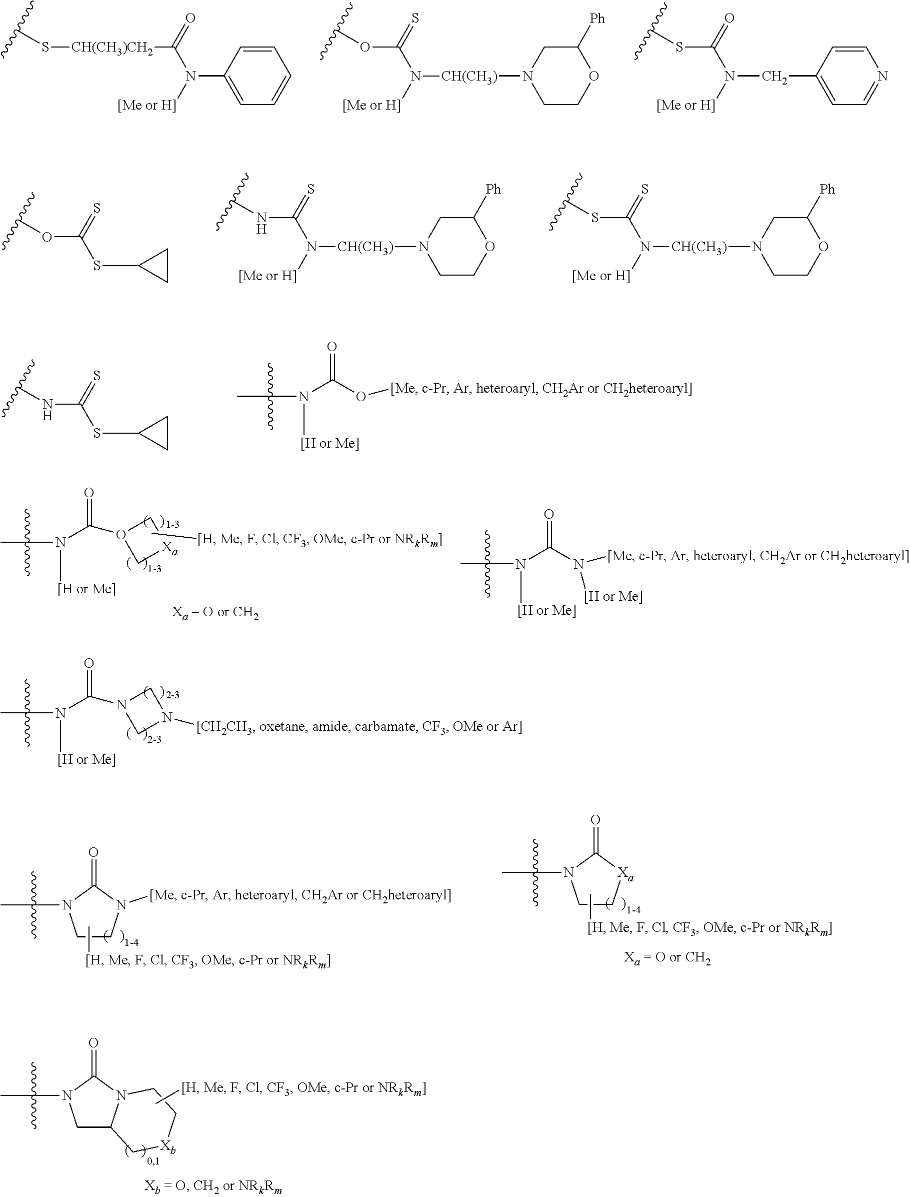

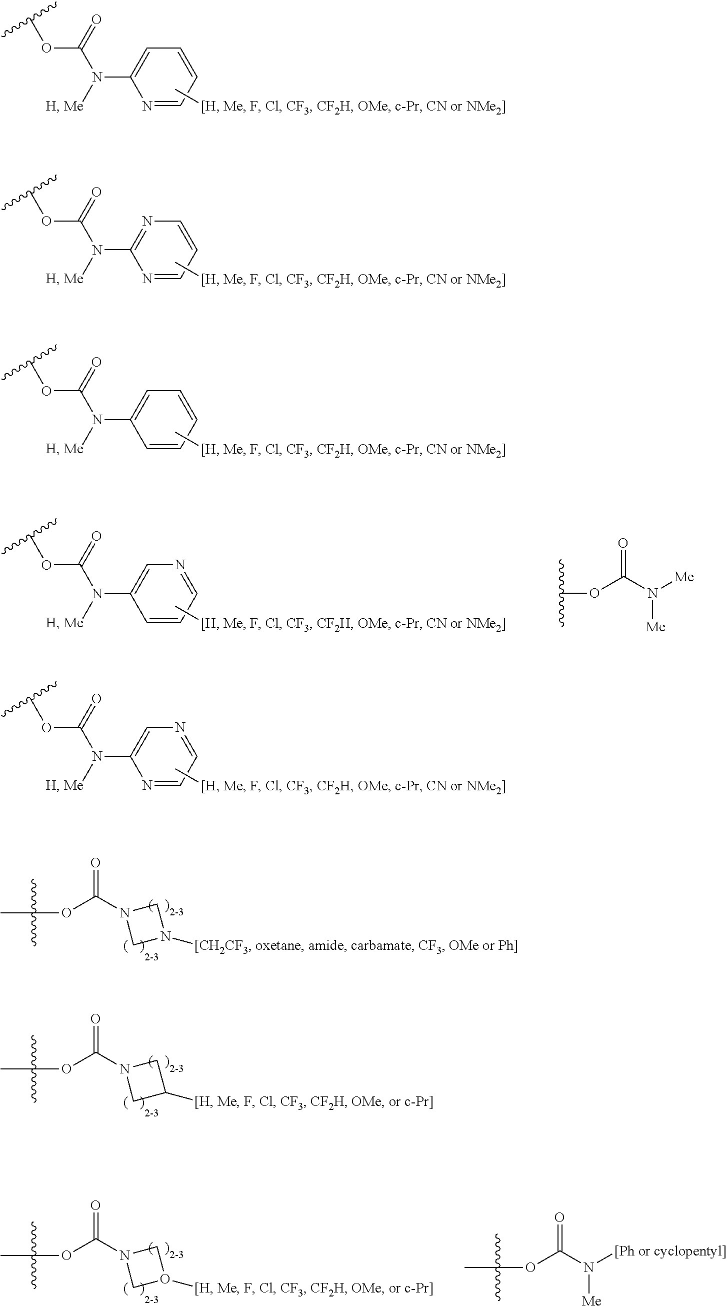

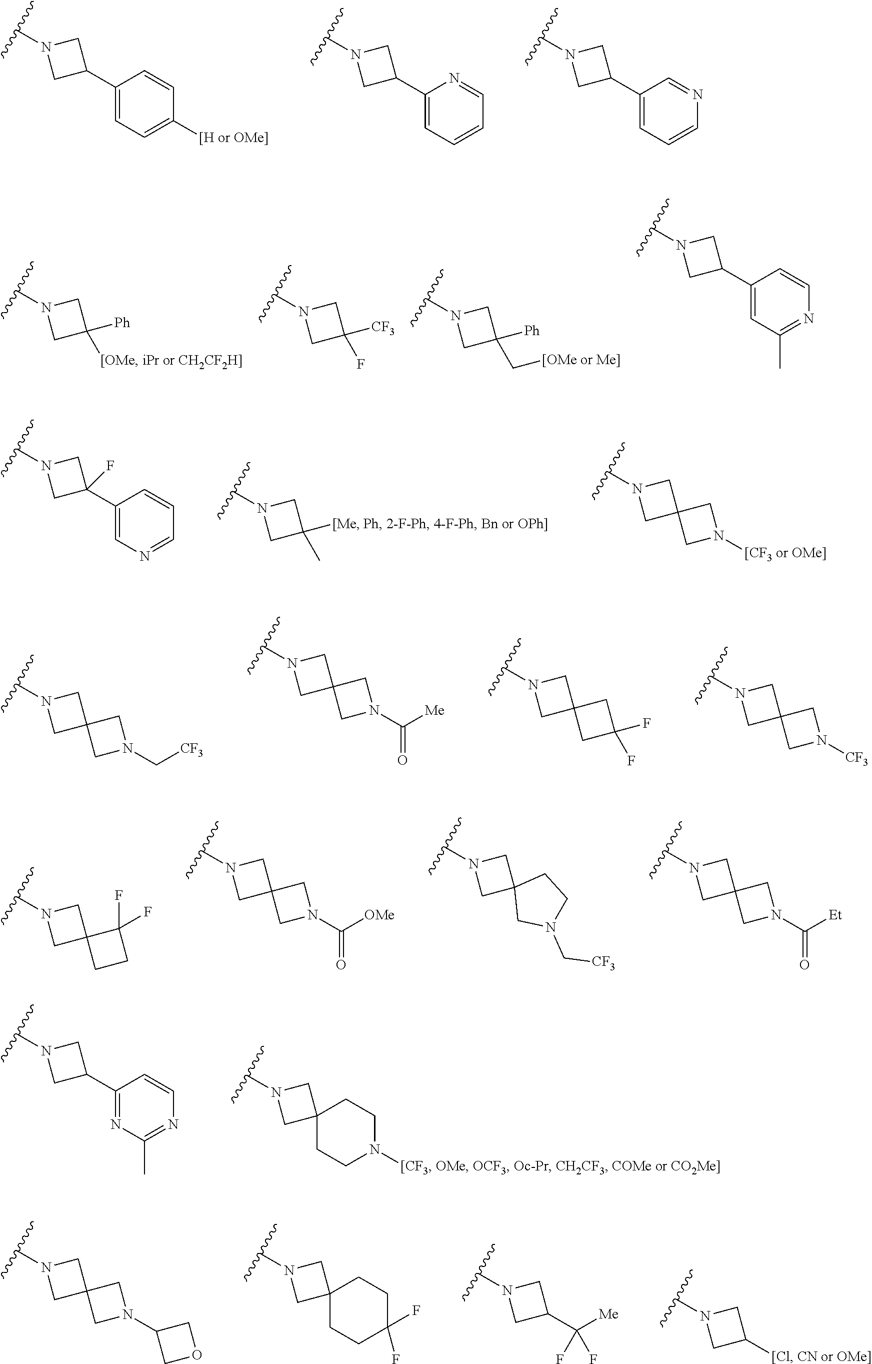

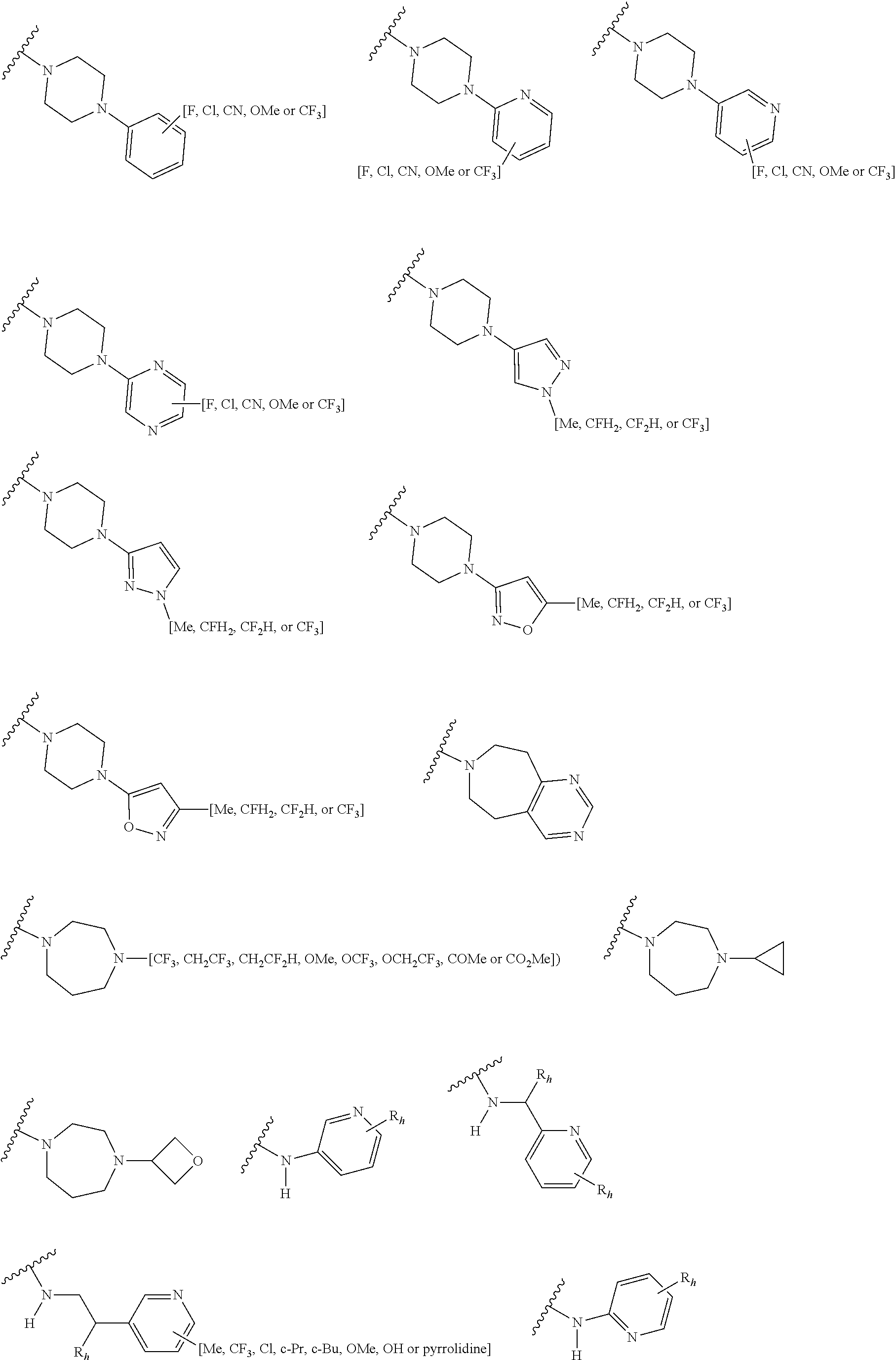

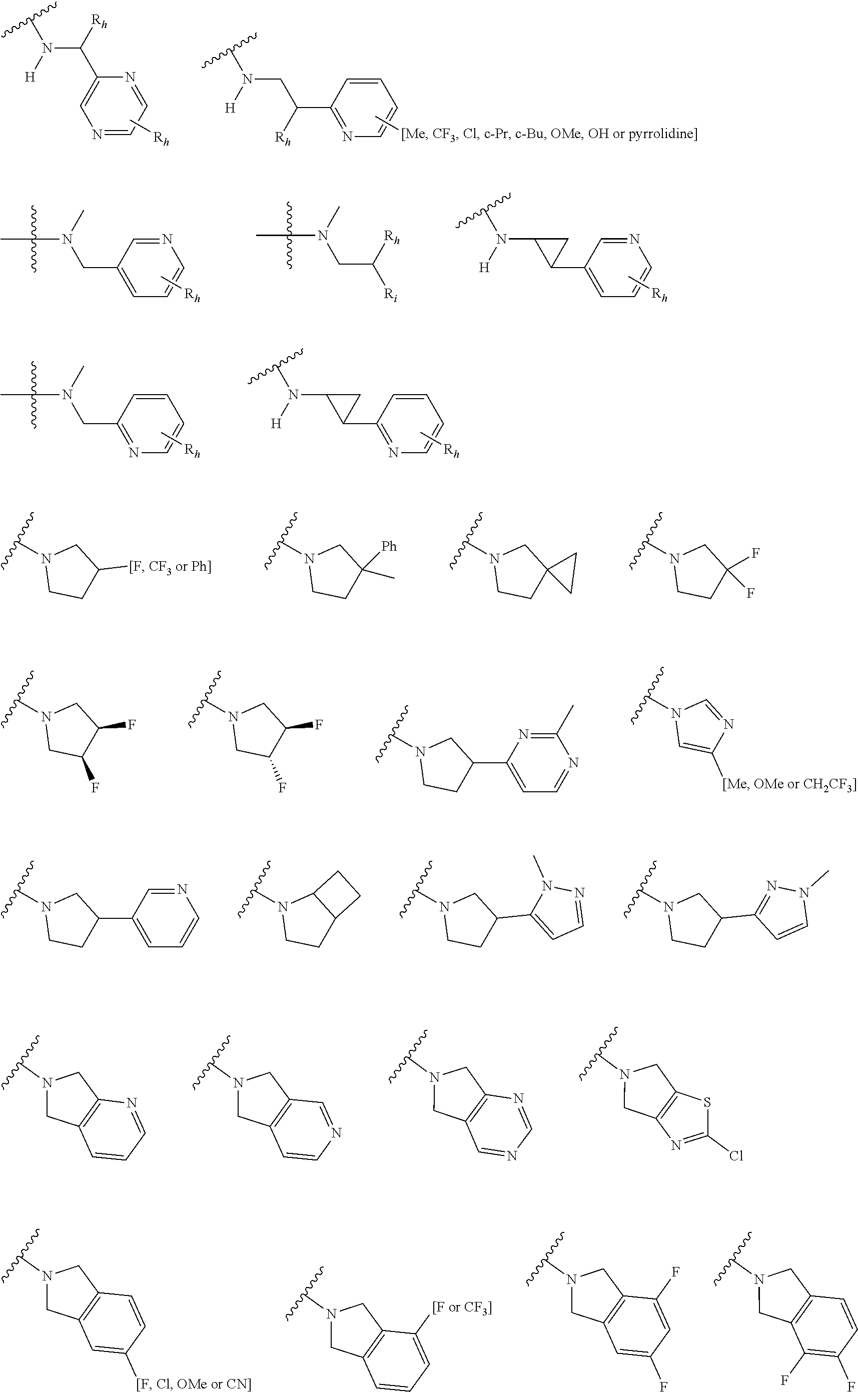

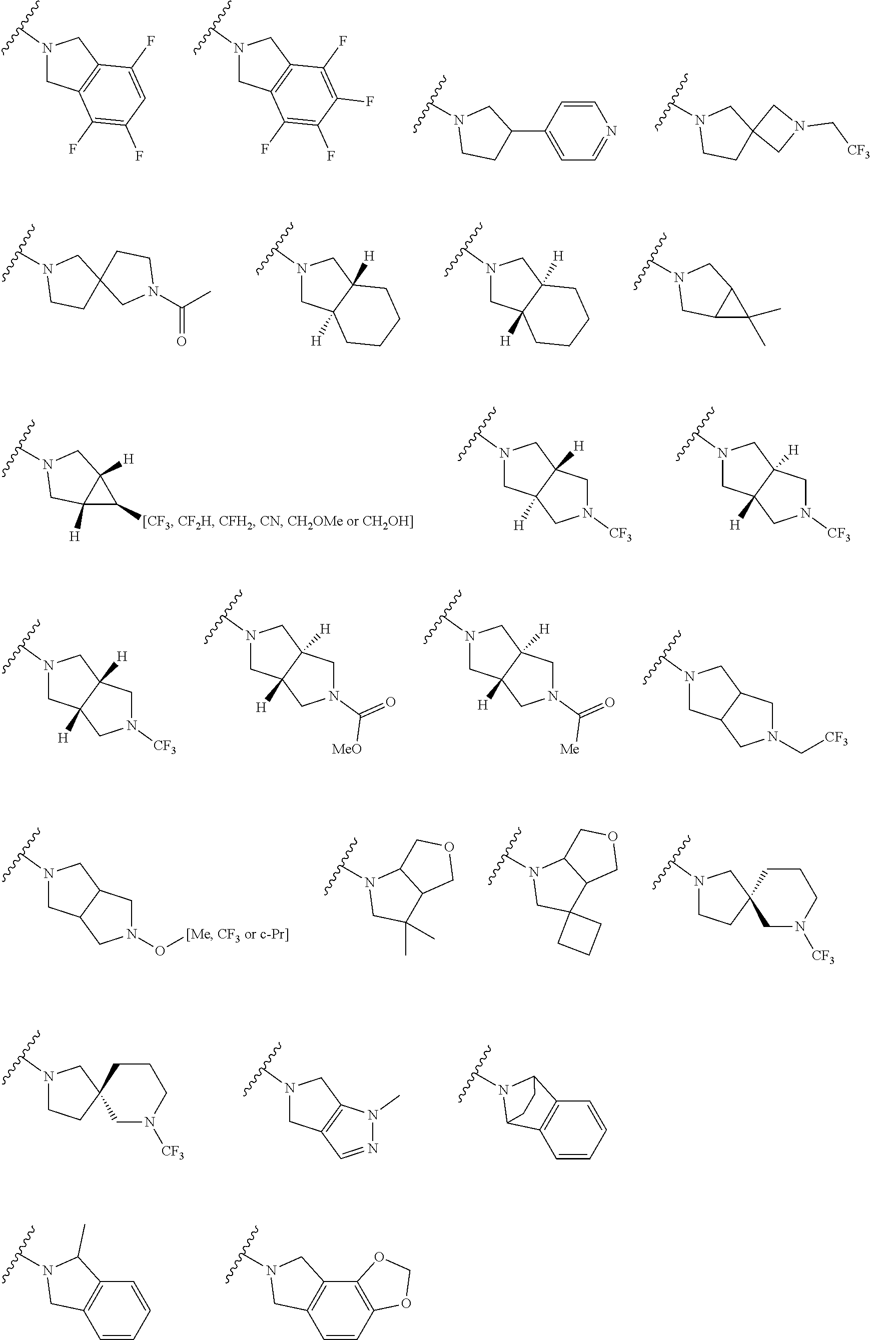

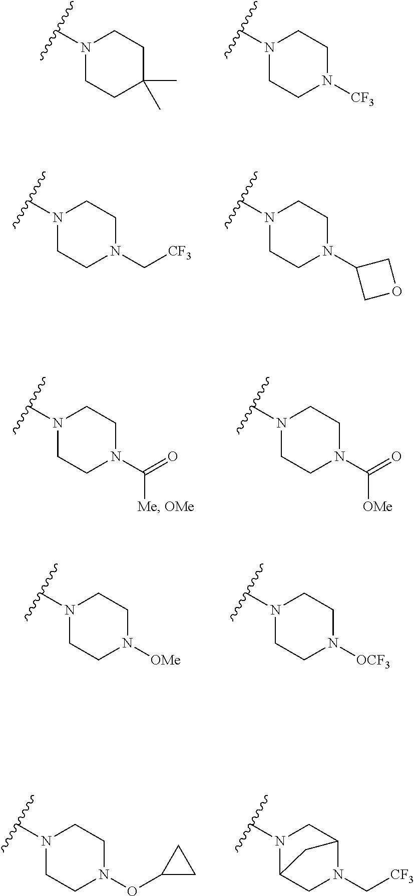

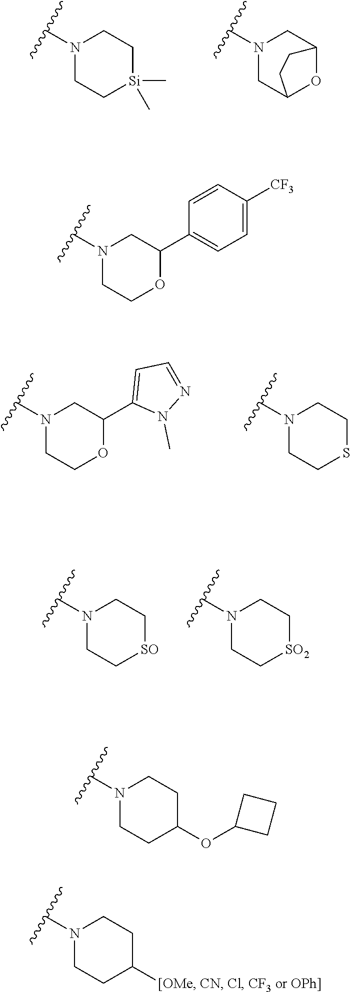

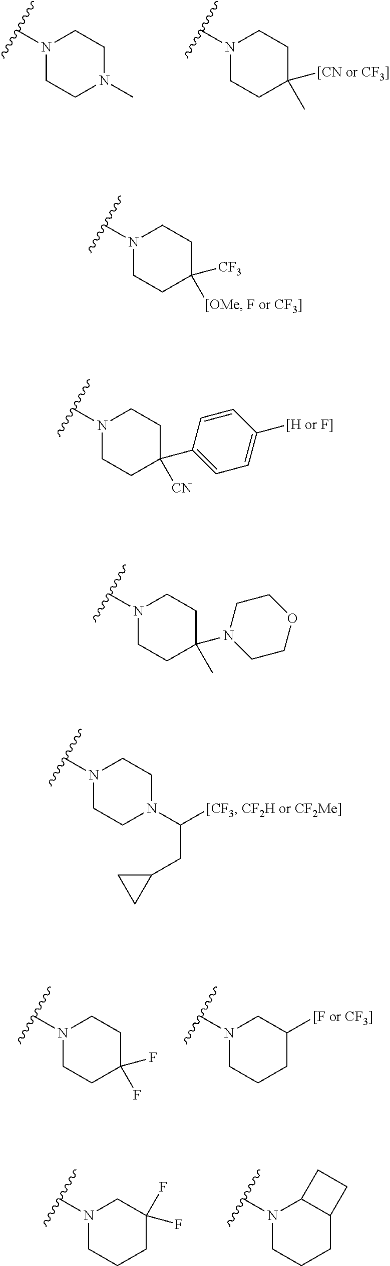

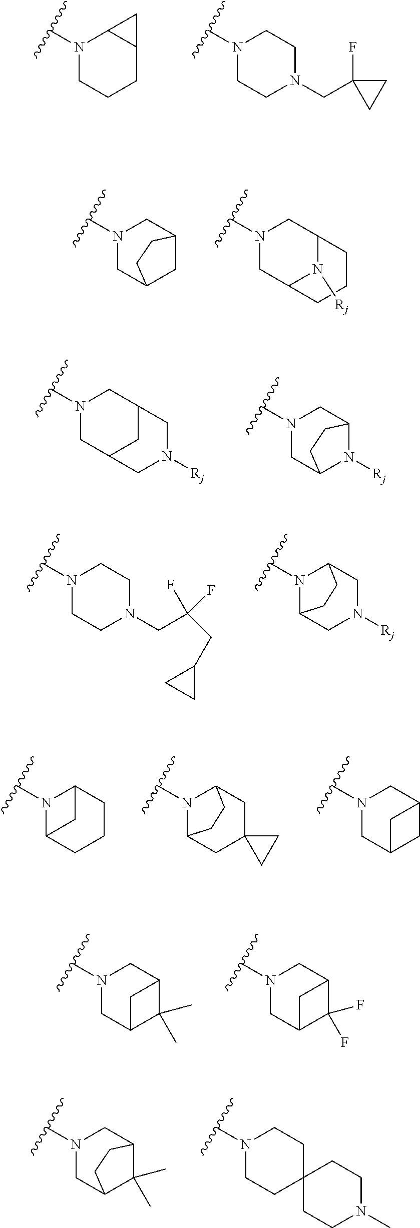

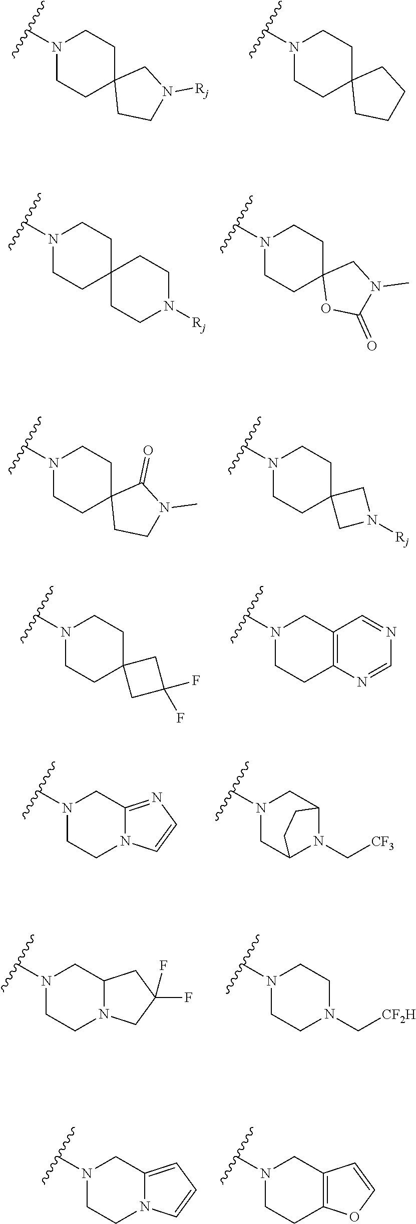

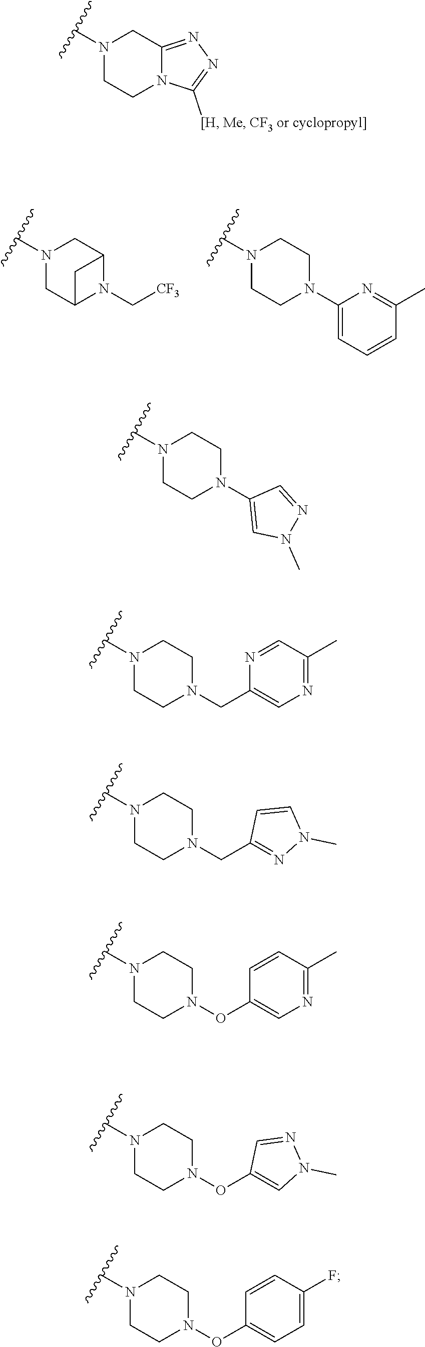

and where R₁′ is selected from

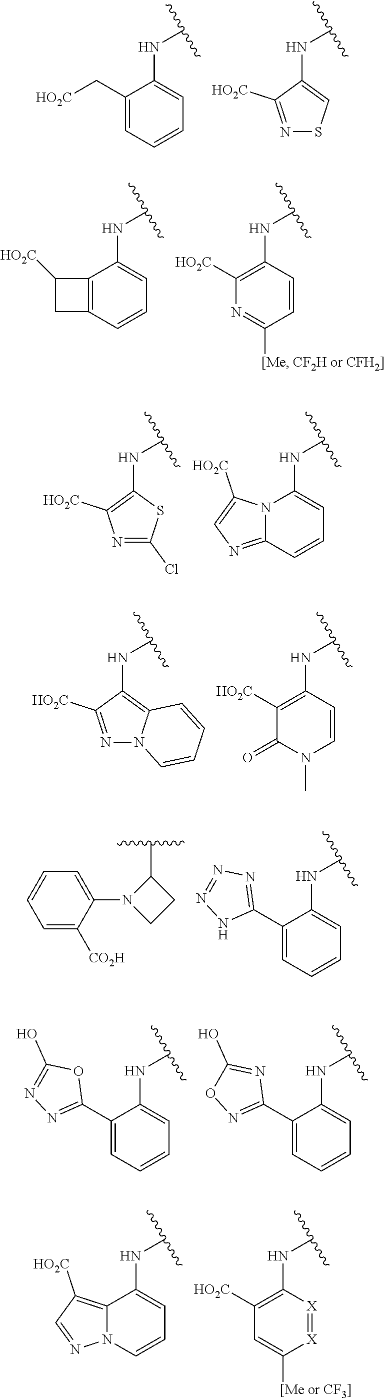

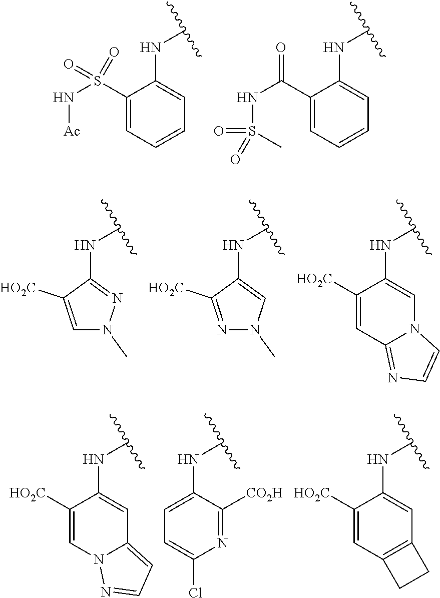

The following compounds of Formula (5) represent various embodiments of the present invention where R₅ has been varied in a structure where all other atoms have been set. The carbon atom marked with * is a chiral center and exists as a (R)- and (S)-racemic mixture or as either (R)- or (S)-enantiomer. The listing of substituents within brackets (in Formula (5) or for a listed embodiment of R₅) indicates individual compounds containing one of each of the substituents. Where present in embodiments of R₅, each R₁₈ is independently H, C₁-C₄ alkyl, C₃-C₇ cycloalkyl, halogen, CN, CF₃, OCF₃, CFH₂ or CF₂H; and each X is independently N or CH. All chiral centers in the below R₅ structures that are not specified, exist as a (R)- and (S)-racemic mixture or as either (R)- or (S)-enantiomer.

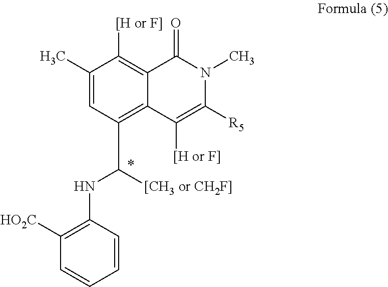

where R₅ is selected from:

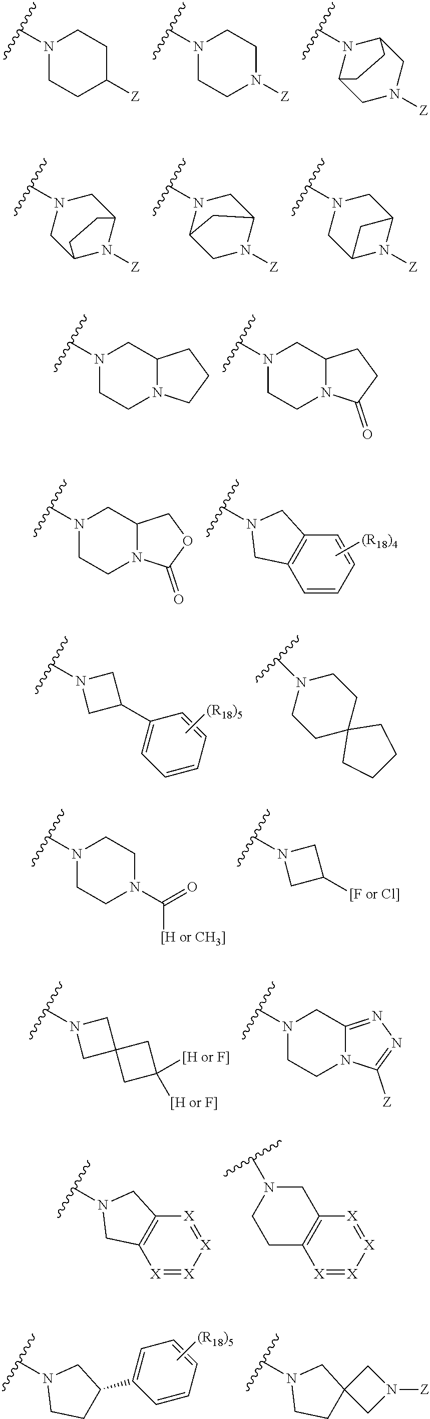

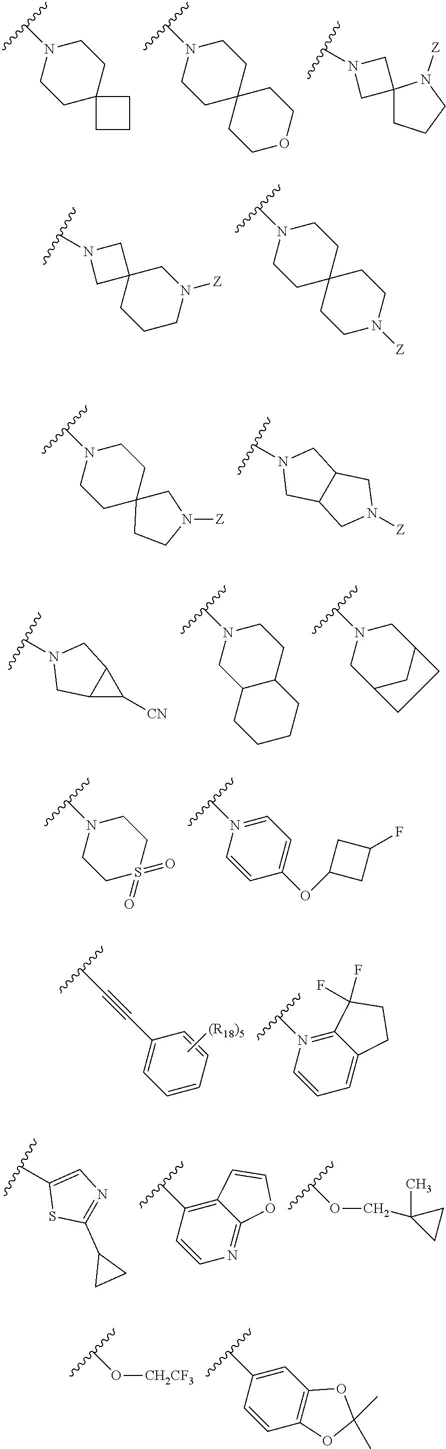

where each Z is independently selected from the following:

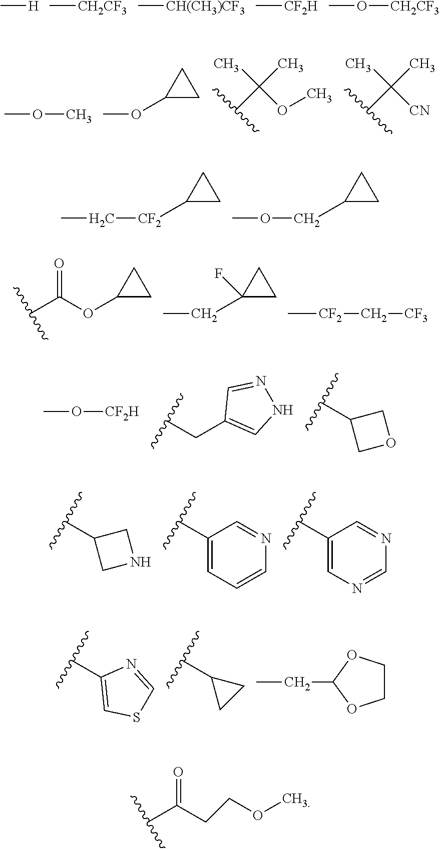

The following compounds of Formula (6) represent various embodiments of the present invention where R₅ has been varied in a structure where all other atoms have been set. The carbon atom marked with * is a chiral center and exists as a (R)- and (S)-racemic mixture or as either (R)- or (S)-enantiomer. The listing of substituents within brackets (in Formula (6) or for a listed embodiment of R₅) indicates individual compounds containing one of each of the substituents. Where a hashed line ( [symbol] ) is present in embodiments of R₅, the bond can be either saturated or unsaturated. All chiral centers in the below R₅ structures that are not specified, exist as a (R)- and (S)-racemic mixture or as either (R)- or (S)-enantiomer.

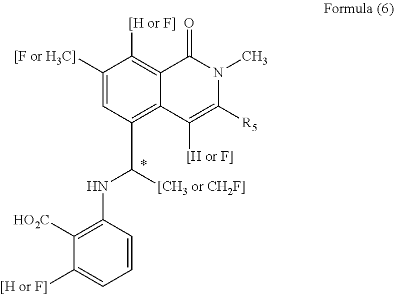

where R₅ is selected from:

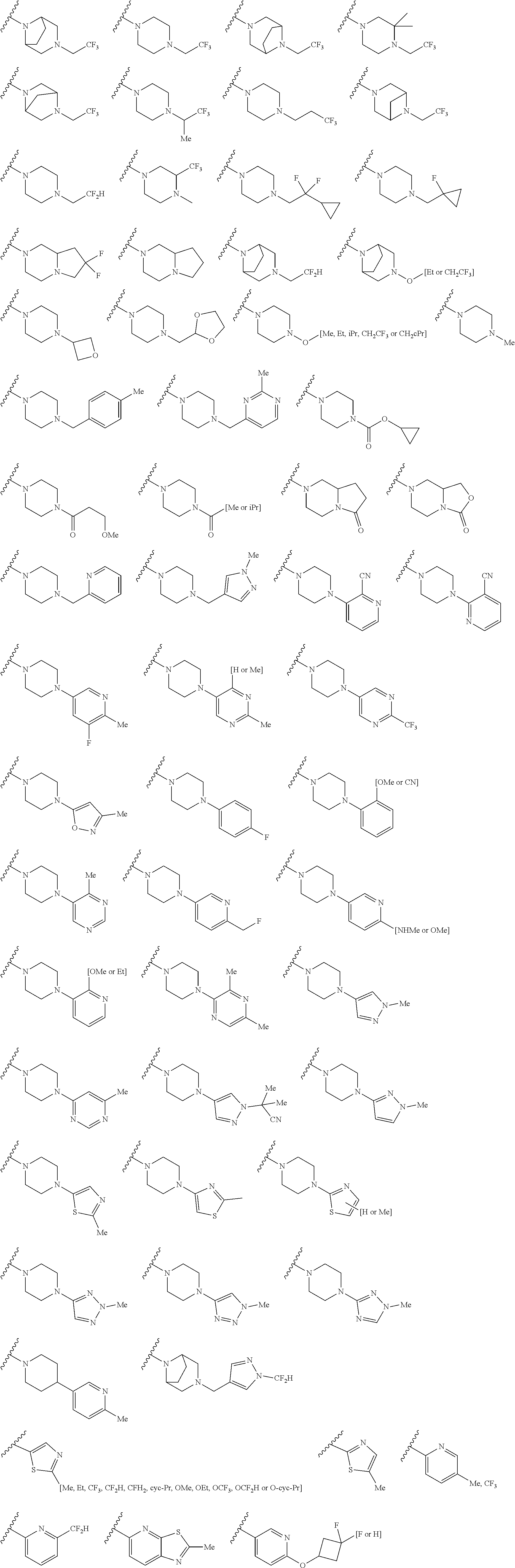

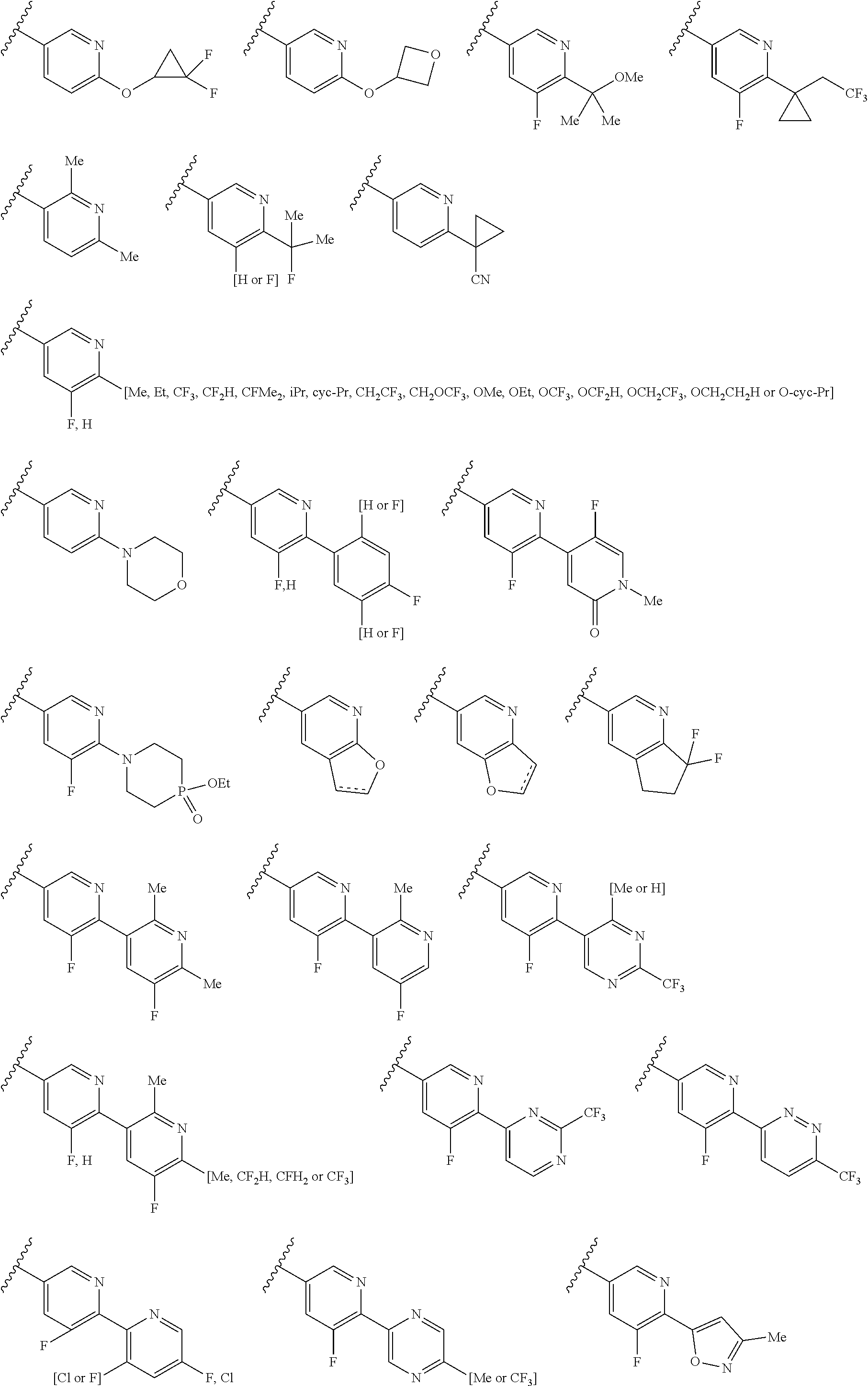

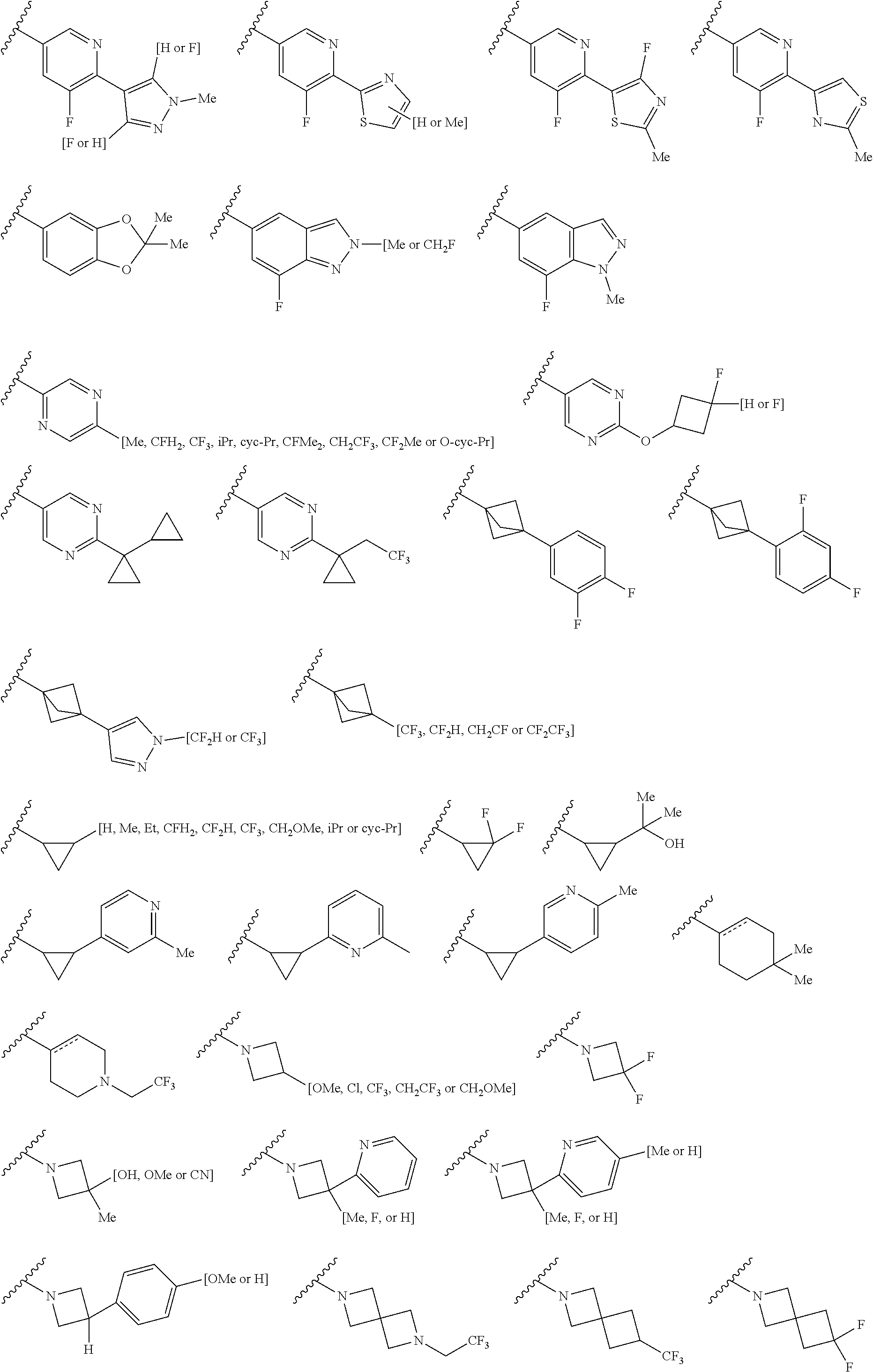

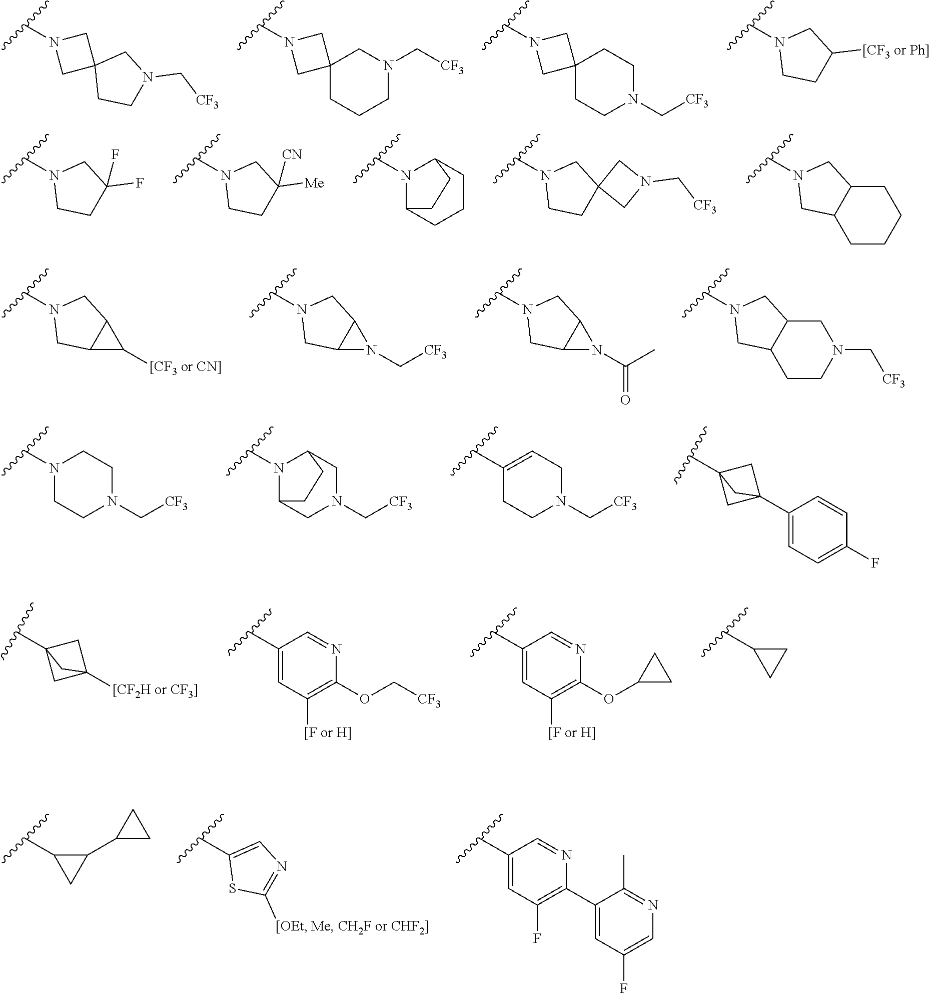

### Experimental

All commercially available solvents and reagents were used as received. All ¹H NMR spectra were recorded using a Bruker Avance III HD 300 MHz or Bruker Avance III HD 400 MHz. MS samples were analyzed on a Shimadzu LCMS-2020 mass spectrometer with electrospray ionization operating in positive and negative ion mode. Samples were introduced into the mass spectrometer using chromatography. All final products had a purity of ≥90%, unless specified otherwise in the experimental details. HPLC purity was measured on a Shimadzu Acquity HPLC system.

The following represents acronyms used in the experimental section for well-known chemical solvents, reagents, parameters and techniques:
¹H NMR: proton nuclear magnetic resonance spectroscopy
ACN: acetonitrile
AcOH: acetic acid
c-Bu: cyclobutyl
c-Pr: cyclopropyl
CeCl₃: cerium (III) chloride
CH₂Cl₂: dichloromethane
CHCl₃: chloroform
Cs₂CO₃: cesium carbonate
DBAD: di-tert-butyl azodicarboxylate
DCM: dichloromethane
DIBAL: diisobutylaluminum hydride
DIEA: N,N-diisopropylethylamine
DMF: N,N-dimethylformamide
DMSO: dimethyl sulfoxide
DTAD: di-tert-butyl azodicarboxylate
EA: ethyl acetate
ee: enantiomeric excess
Et₂O: diethyl ether
Et₃N: triethylamine
EtOAc: ethyl acetate
EtOH: ethanol
FA: formic acid
h: hours
H₂O: water
HATU: 1-[Bis(dimethylamino)methylene]-1H-1,2,3-triazolo[4,5-b]pyridinium 3-oxide hexafluorophosphate
HCl: hydrochloric acid
Hex: hexanes
HPLC: high-performance liquid chromatography
IPA: isopropanol
K₂CO₃: potassium carbonate
KOAc: potassium acetate
LiOH: lithium hydroxide
mCPBA: meta-chloroperoxybenzoic acid
Me: methyl
MeCN: acetonitrile
MeOH: methanol
mg: milligram
min: minutes
mL: milliliter
MsCl: methanesulfonyl chloride
Ms₂O: methanesulfonic anhydride
NaBH₄: sodium borohydride
N₂: nitrogen
NaCl: sodium chloride
Na₂CO₃: sodium carbonate
NaH: sodium hydrideNaOH: sodium hydroxide
NaHCO₃: sodium bicarbonate
NaH₂PO₄: monosodium phosphate
Na₂SO₄: sodium sulfate
NH₃: ammonia
NH₄HCO₃: ammonium bicarbonate
NMP: N-methylpyrrolidone
Oxetane: 4-membered ring containing 3 carbon ring atoms and 1 oxygen ring atom.
PBr₃: phosphorous tribromide
PCl₅: phosphorous pentachloride
Pd-PEPPSI-IHeptCl 3-chloropyridine: dichloro[1,3-bis(2,6-di-4-heptylphenyl) imidazol-2-ylidene](3-chloropyridyl)palladium(II)
Pd(dppf)Cl₂: (1,1′-bis(diphenylphosphino)ferrocene)palladium(II) dichloride
Pd(PPh₃)₄: tetrakis(triphenylphosphine)palladium(0)
Pd₂(dba)₃: tris(dibenzylideneacetone)dipalladium(0)
PdCl₂(PPh₃)₂: bis(triphenylphosphine)palladium(II) dichloridePE: petroleum ether
POCl₃: phosphorus oxychloride
PPh₃: triphenylphosphine
Prep: preparative
RuPhos: 2-dicyclohexylphosphino-2′,6′-diisopropoxybiphenyl
TEA: triethylamine
TFA: trifluoroacetic acid
THF: tetrahydrofuran
Ti(Oi-Pr)₄: Titanium (IV) isopropoxide
TLC: thin-layer chromatography
Xantphos: 4,5-bis(diphenylphosphino)-9,9-dimethylxanthene

### EXAMPLES

### Example 1: 2-((1-(3-(4,4-dimethylpiperidin-1-yl)-2,7-dimethyl-1-oxo-1,2-dihydroisoquinolin-5-yl)ethyl)amino)benzoic acid (Enantiomer 1)

### Example 2: 2-((1-(3-(4,4-dimethylpiperidin-1-yl)-2,7-dimethyl-1-oxo-1,2-dihydroisoquinolin-5-yl)ethyl)amino)benzoic acid (Enantiomer 2)

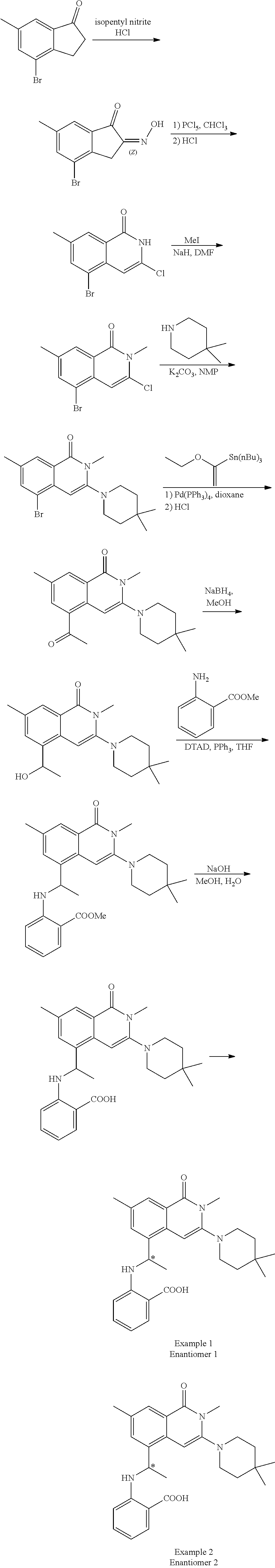

### Step 1: Preparation of (2Z)-4-bromo-2-(hydroxyimino)-6-methyl-3H-inden-1-one

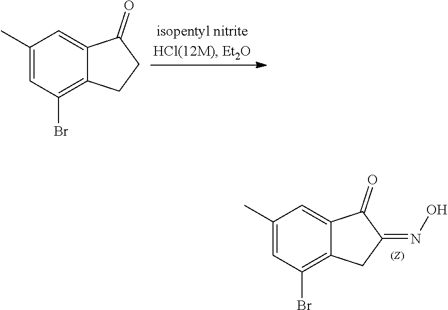

To a stirred solution of 4-bromo-6-methyl-2,3-dihydroinden-1-one (2 g, 8.89 mmol) in 12 M aqueous HCl (10 mL) and Et₂O (10 mL) was added 3-methylbutyl nitrite (1.25 g, 10.66 mmol) slowly dropwise at 0° C. The resulting solution was stirred for 4 h at room temperature. The mixture was cooled to 0° C. and the precipitated solids were collected by filtration and washed with H₂O (3×50 mL) and Et₂O (2×20 mL). The collected solids were concentrated and dried under vacuum. The crude product was used in the next step directly without further purification. This resulted in (2Z)-4-bromo-2-(hydroxyimino)-6-methyl-3H-inden-1-one (1.5 g, 66%) as an off-white solid. MS: (ES⁺) m/z=253.9 [M+H]⁺.

### Step 2: Preparation of 5-bromo-3-chloro-7-methyl-2H-isoquinolin-1-one

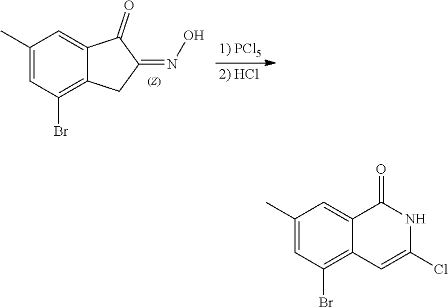

To a stirred solution of (2Z)-4-bromo-2-(hydroxyimino)-6-methyl-3H-inden-1-one (1.5 g, 5.90 mmol) in CHCl₃ (30 mL) was slowly added PCl₅ (2.46 g, 11.81 mmol) in portions at 0° C. The resulting mixture was stirred for 3 h at room temperature and then concentrated under reduced pressure. To the crude product was added 4 M HCl in 1,4-dioxane (30 mL). The resulting solution was stirred overnight at room temperature and then concentrated under reduced pressure. The residue was purified by trituration with 5:1 PE/EtOAc to afford 5-bromo-3-chloro-7-methyl-2H-isoquinolin-1-one (1 g, 61%) as a yellow solid. MS: (ES⁻) m/z=269.9 [M−1]⁻.

### Step 3: Preparation of 5-bromo-3-chloro-2,7-dimethylisoquinolin-1-one

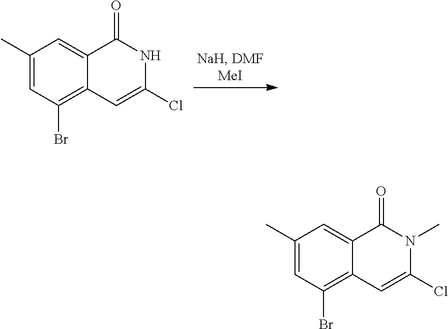

To a stirred solution of 5-bromo-3-chloro-7-methyl-2H-isoquinolin-1-one (1.3 g, 4.77 mmol) in DMF (10 mL) was slowly added NaH (0.17 g, 7.16 mmol) in portions at 0° C. The resulting solution was stirred for 20 min at 0° C. Iodomethane (0.81 g, 5.72 mmol) was slowly added dropwise at 0° C. and the resulting solution was stirred overnight at room temperature. The reaction was quenched with water (40 mL) and the resulting mixture was extracted with EtOAc (3×50 mL). The combined organic layers were washed with brine (3×40 mL), dried over anhydrous Na₂SO₄, and then concentrated under reduced pressure. The residue was purified by silica gel column chromatography (PE/EA=4:1) to afford 5-bromo-3-chloro-2,7-dimethylisoquinolin-1-one (900 mg, 65%) as a reddish brown solid. MS: (ES⁺) m/z=286.0 [M+H]⁺.

### Step 4: Preparation of 5-bromo-3-(4,4-dimethylpiperidin-1-yl)-2,7-dimethylisoquinolin-1-one

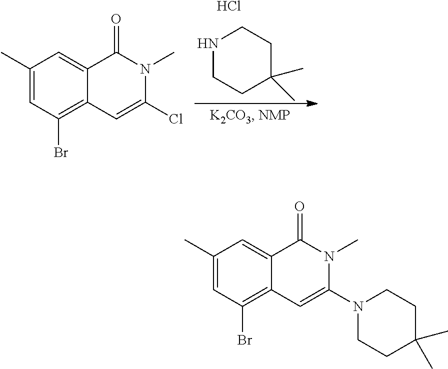

A mixture of 5-bromo-3-chloro-2,7-dimethylisoquinolin-1-one (900 mg, 3.14 mmol), 4,4-dimethylpiperidine (940 mg, 6.28 mmol) and K₂CO₃ (1.30 g, 9.42 mmol) in NMP (15 mL) was stirred overnight under a nitrogen atmosphere at 140° C. The mixture was cooled to room temperature, diluted with water (40 mL), and extracted with EtOAc (3×80 mL). The combined organic layers were washed with brine (3×40 mL), dried over anhydrous Na₂SO₄, and then concentrated under reduced pressure. The residue was purified by silica gel column chromatography (PE/EA=3:1) to afford 5-bromo-3-(4,4-dimethylpiperidin-1-yl)-2,7-dimethylisoquinolin-1-one (700 mg, 61%) as a yellow solid. MS: (ES⁺) m/z=363.1 [M+H]⁺.

### Step 5: Preparation of 5-acetyl-3-(4,4-dimethylpiperidin-1-yl)-2,7-dimethylisoquinolin-1-one

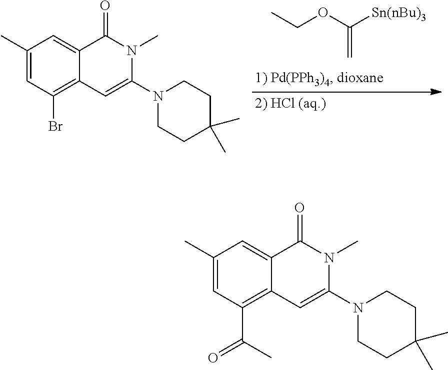

A mixture of 5-bromo-3-(4,4-dimethylpiperidin-1-yl)-2,7-dimethylisoquinolin-1-one (700 mg, 1.93 mmol), tributyl(1-ethoxyethenyl)stannane (835 mg, 2.31 mmol) and Pd(PPh₃)₄ (222 mg, 0.19 mmol) in anhydrous 1,4-dioxane (12 mL) was stirred overnight at 100° C. under a nitrogen atmosphere. The reaction mixture was cooled to room temperature. Aqueous 1 N HCl solution (2 mL) was added, the mixture was stirred for 20 min at 50° C., and then the mixture was cooled to room temperature. The mixture was diluted with water (20 mL) and extracted with ethyl acetate (3×30 mL). The combined organic layers were washed with water (3×20 mL) and brine (20 mL), dried over anhydrous sodium sulfate, and concentrated under reduced pressure. The residue was purified by prep TLC (PE/EA=2:1) to afford 5-acetyl-3-(4,4-dimethylpiperidin-1-yl)-2,7-dimethylisoquinolin-1-one (550 mg, 87%) as a yellow solid. MS: (ES⁺) m/z=327.1 [M+H]⁺.

### Step 6: Preparation of 3-(4,4-dimethylpiperidin-1-yl)-5-(1-hydroxyethyl)-2,7-dimethylisoquinolin-1-one

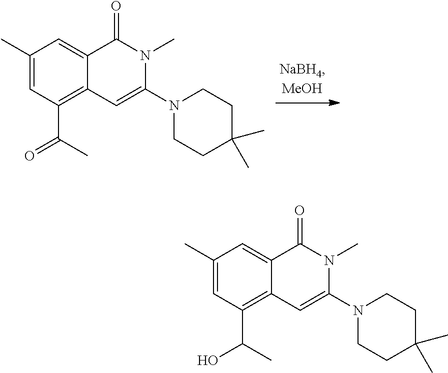

To a stirred solution of 5-acetyl-3-(4,4-dimethylpiperidin-1-yl)-2,7-dimethylisoquinolin-1-one (550 mg, 1.69 mmol) in MeOH (10 mL) was slowly added NaBH₄ (127 mg, 3.37 mmol) in portions at 0° C. The resulting solution was stirred for 2 h at room temperature. The resulting mixture was quenched with H₂O (20 mL) and extracted with EtOAc (3×80 mL). The combined organic layers were washed with brine (2×50 mL), dried over anhydrous Na₂SO₄, and concentrated under reduced pressure. The residue was purified by silica gel column chromatography (PE/EA=2:1) to afford 3-(4,4-dimethylpiperidin-1-yl)-5-(1-hydroxyethyl)-2,7-dimethylisoquinolin-1-one (400 mg, 72%) as a light yellow solid. MS: (ES⁺) m/z=329.2 [M+H]⁺.

### Step 7: Preparation of methyl 2-((1-(3-(4,4-dimethylpiperidin-1-yl)-2,7-dimethyl-1-oxo-1,2-dihydroisoquinolin-5-yl)ethyl)amino)benzoate

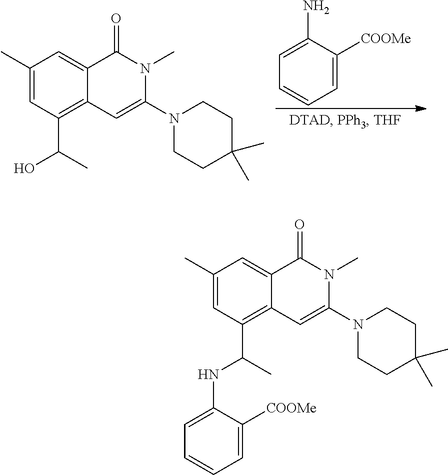

To a solution of 3-(4,4-dimethylpiperidin-1-yl)-5-(1-hydroxyethyl)-2,7-dimethylisoquinolin-1-one (400 mg, 1.22 mmol), methyl anthranilate (920 mg, 6.090 mmol), and PPh₃ (798 mg, 3.05 mmol) in THF (10 mL) was added a solution of (E)-N-[[(tert-butoxy)carbonyl]imino](tert-butoxy) formamide (560 mg, 2.44 mmol) in tetrahydrofuran (1 mL) at 0° C. under a nitrogen atmosphere. The resulting mixture was stirred overnight at room temperature and then concentrated under reduced pressure. The residue was purified by prep TLC (PE/EA=6:1) to afford methyl 2-((1-(3-(4,4-dimethylpiperidin-1-yl)-2,7-dimethyl-1-oxo-1,2-dihydroisoquinolin-5-yl)ethyl)amino)benzoate (280 mg, 49%) as a light yellow solid. MS: (ES⁺) m/z=462.2 [M+H]⁺.

### Step 8: Preparation of 2-((1-(3-(4,4-dimethylpiperidin-1-yl)-2,7-dimethyl-1-oxo-1,2-dihydroisoquinolin-5-yl)ethyl)amino)benzoic acid

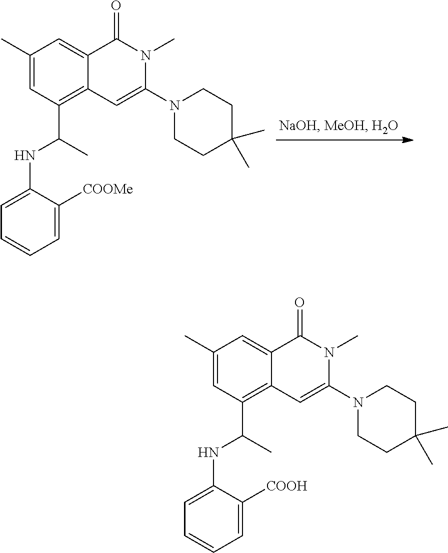

To a stirred solution of methyl 2-((1-(3-(4,4-dimethylpiperidin-1-yl)-2,7-dimethyl-1-oxo-1,2-dihydroisoquinolin-5-yl)ethyl)amino)benzoate (280 mg, 0.61 mmol) and NaOH (242 mg, 6.07 mmol) in MeOH (5 mL) and H₂O (5 mL). The resulting mixture was stirred overnight at 50° C., cooled to room temperature, and then acidified to pH 5-6 with aqueous 2 M HCl. The resulting mixture was extracted with ethyl acetate (3×60 mL). The combined organic layers were washed with water (2×30 mL), dried over anhydrous Na₂SO₄, and then concentrated under reduced pressure. The residue was purified by prep TLC (DCM/MeOH=15:1) to afford 2-((1-(3-(4,4-dimethylpiperidin-1-yl)-2,7-dimethyl-1-oxo-1,2-dihydroisoquinolin-5-yl)ethyl)amino)benzoic acid (150 mg, 55%) as an off-white solid. MS: (ES⁺) m/z=448.2 [M+H]⁺.

### Step 9: Preparation of 2-((1-(3-(4,4-dimethylpiperidin-1-yl)-2,7-dimethyl-1-oxo-1,2-dihydroisoquinolin-5-yl)ethyl)amino)benzoic acid

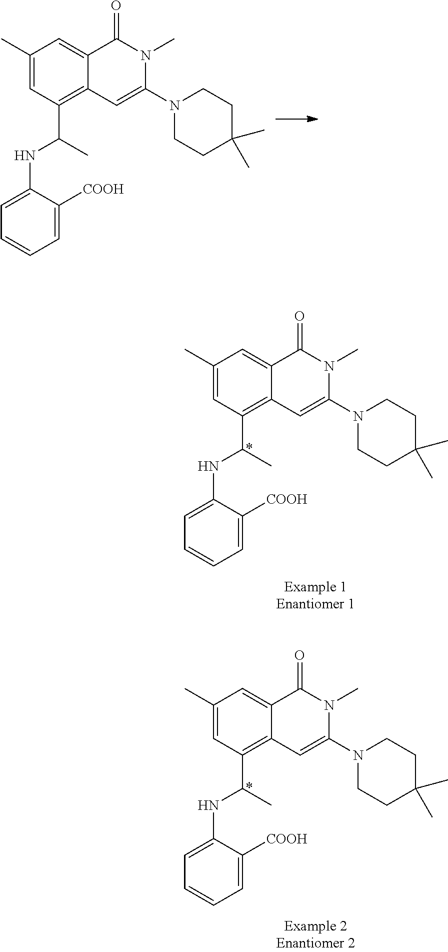

A racemic mixture of 2-((1-(3-(4,4-dimethylpiperidin-1-yl)-2,7-dimethyl-1-oxo-1,2-dihydroisoquinolin-5-yl)ethyl)amino)benzoic acid (150 mg, 0.34 mmol) was separated by prep chiral HPLC (Column: CHIRALPAK IC-3, 2*25 cm, 5 um; Mobile Phase A: Hex (0.1% FA)-HPLC, Mobile Phase B: IPA-HPLC; Flow rate: 20 mL/min; Gradient: 20% B to 20% B in 20 min; Wavelengths: 220/254 nm) to afford each enantiomer of 2-((1-(3-(4,4-dimethylpiperidin-1-yl)-2,7-dimethyl-1-oxo-1,2-dihydroisoquinolin-5-yl)ethyl)amino)benzoic acid:

2-((1-(3-(4,4-dimethylpiperidin-1-yl)-2,7-dimethyl-1-oxo-1,2-dihydroisoquinolin-5-yl)ethyl)amino)benzoic acid Enantiomer 1, (20.9 mg, 14%, ˜99.9% ee, white solid). MS: (ES⁺) m/z=448.2 [M+H]⁺. ¹H NMR: (300 MHz, DMSO-d6) δ 12.76 (s, 1H), 8.40 (s, 1H), 7.94-7.71 (m, 2H), 7.40 (d, J=2.0 Hz, 1H), 7.23-7.12 (m, 1H), 6.57-6.48 (m, 1H), 6.39 (d, J=8.9 Hz, 2H), 5.22-5.10 (m, 1H), 3.47 (s, 3H), 3.00-2.80 (m, 4H), 2.32 (s, 3H), 1.71-1.44 (m, 7H), 1.00 (s, 6H).

2-((1-(3-(4,4-dimethylpiperidin-1-yl)-2,7-dimethyl-1-oxo-1,2-dihydroisoquinolin-5-yl)ethyl)amino)benzoic acid, Enantiomer 2, 24.1 mg, 16%, 98.6% ee), light yellow solid, MS: (ES⁺) m/z=448.2 [M+H]⁺. ¹H NMR: (300 MHz, DMSO-d6) δ 12.77 (s, 1H), 8.47 (s, 1H), 8.06-7.76 (m, 2H), 7.41 (d, J=1.9 Hz, 1H), 7.23-7.12 (m, 1H), 6.57-6.48 (m, 1H), 6.38 (d, J=5.6 Hz, 2H), 5.22-5.10 (m, 1H), 3.47 (s, 3H), 3.00-2.80 (m, 4H), 2.32 (s, 3H), 1.70-1.46 (m, 7H), 1.00 (s, 6H).

### Example 3: 6-chloro-3-((1-(3-(4,4-dimethylpiperidin-1-yl)-2,7-dimethyl-1-oxo-1,2-dihydroisoquinolin-5-yl)ethyl)amino)picolinic acid (Enantiomer 1)

### Example 4: 6-chloro-3-((1-(3-(4,4-dimethylpiperidin-1-yl)-2,7-dimethyl-1-oxo-1,2-dihydroisoquinolin-5-yl)ethyl)amino)picolinic acid (Enantiomer 2)

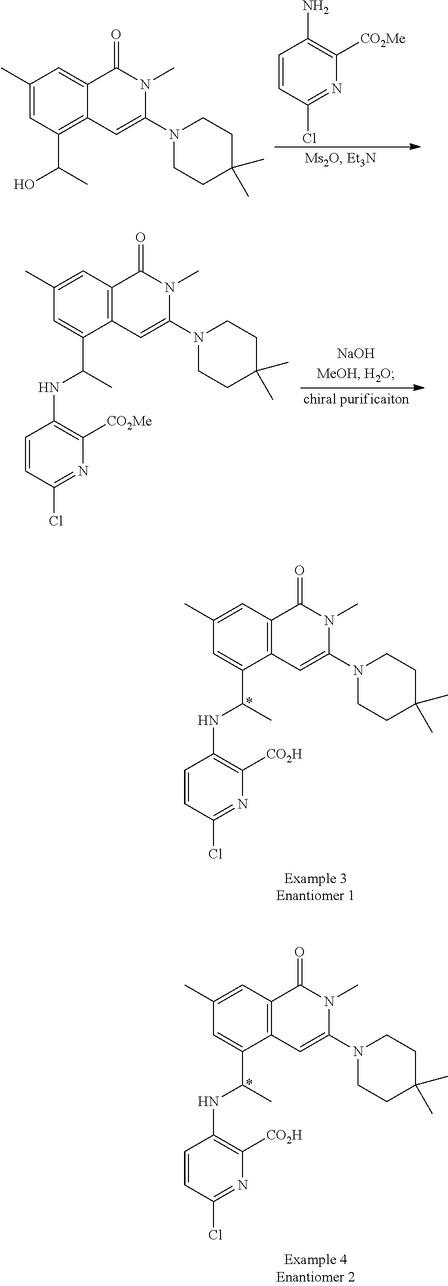

### Step 1: Preparation of methyl 6-chloro-3-((1-(3-(4,4-dimethylpiperidin-1-yl)-2,7-dimethyl-1-oxo-1,2-dihydroisoquinolin-5-yl)ethyl)amino)picolinate

To a stirred solution of 3-(4,4-dimethylpiperidin-1-yl)-5-(1-hydroxyethyl)-2,7-dimethylisoquinolin-1-one (500 mg, 1.52 mmol) and TEA (924 mg, 9.13 mmol) in DCM (10 mL) was added methanesulfonic anhydride (1.06 g, 6.09 mmol) in portions at 0° C. The resulting mixture was stirred for 1 h at 0° C. Methyl 3-amino-6-chloropyridine-2-carboxylate (341 mg, 1.83 mmol) was added and the mixture was stirred for additional 12 h at 50° C. The resulting mixture was diluted with H₂O (50 mL) and extracted with DCM (3×50 mL). The combined organic layers were washed with brine (3×50 mL), dried over anhydrous Na₂SO₄, and then were concentrated under reduced pressure. The residue was purified by reverse phase flash chromatography (Column: Regular C18; 330 g, 20-40 μm; Gradient: 10% to 100% ACN in water over 30 min, flow rate: 100 mL/min) to afford methyl 6-chloro-3-((1-(3-(4,4-dimethylpiperidin-1-yl)-2,7-dimethyl-1-oxo-1,2-dihydroisoquinolin-5-yl)ethyl)amino)picolinate (410 mg, 54%) as a yellow solid. MS: (ES⁺) m/z=497.2 [M+H]⁺.

### Step 2: Preparation of 6-chloro-3-((1-(3-(4,4-dimethylpiperidin-1-yl)-2,7-dimethyl-1-oxo-1,2-dihydroisoquinolin-5-yl)ethyl)amino)picolinic acid (Enantiomers 1 and 2)

To a stirred solution of methyl 6-chloro-3-((1-(3-(4,4-dimethylpiperidin-1-yl)-2,7-dimethyl-1-oxo-1,2-dihydroisoquinolin-5-yl)ethyl)amino)picolinate (290 mg, 0.58 mmol) in MeOH (6 mL) and H₂O (3 mL) was added NaOH (233 mg, 5.83 mmol). The resulting mixture was stirred for 12 h at room temperature. The mixture was acidified to pH 5 with aqueous 1 N HCl and diluted with H₂O (20 mL). The resulting mixture was extracted with EA (3×30 mL). The combined organic layers were washed with brine (30 mL), dried over anhydrous Na₂SO₄, and then concentrated under reduced pressure. The residue was purified by reverse flash chromatography (Column: Regular C18; 330 g, 20-40 μm; Gradient: 10% to 100% ACN in water over 30 min, flow rate: 100 mL/min), then by CHIRAL-HPLC with the following conditions: Column: CHIRALPAK IC-3, 4.6*50 mm, 3 μm; Mobile phase: hexane (0.1% TFA): EtOH=80:20; Flow rate: 20 mL/min), and then further purified by prep HPLC (Column: XBridge Shield RP18 OBD Column; 30*150 mm, 5 μm; Gradient: Mobile Phase A: Water (10 mmol/L NH₄HCO₃+0.1% NH₃·H₂O), Mobile Phase B: ACN; 10% to 100% ACN 30 min, 25% B to 55% in 9 min, flow rate: 60 mL/min) to afford each enantiomer 6-chloro-3-((1-(3-(4,4-dimethylpiperidin-1-yl)-2,7-dimethyl-1-oxo-1,2-dihydroisoquinolin-5-yl)ethyl)amino)picolinic acid as white solids:

6-Chloro-3-((1-(3-(4,4-dimethylpiperidin-1-yl)-2,7-dimethyl-1-oxo-1,2-dihydroisoquinolin-5-yl)ethyl)amino)picolinic acid Enantiomer 1 (16.6 mg, 5% yield, >99% ee). ¹H-NMR (300 MHz, Methanol-d4) δ 7.97 (s, 1H), 7.52 (d, J=1.9 Hz, 1H), 7.09 (d, J=8.8 Hz, 1H), 6.79 (d, J=8.9 Hz, 1H), 6.48 (s, 1H), 5.05-5.15 (m, 1H), 3.61 (s, 3H), 3.10-2.81 (m, 4H), 2.37 (s, 3H), 1.65 (d, J=6.7 Hz, 7H), 1.06 (s, 6H); MS: (ES⁺) m/z=483.1 [M+H]⁺.

6-Chloro-3-((1-(3-(4,4-dimethylpiperidin-1-yl)-2,7-dimethyl-1-oxo-1,2-dihydroisoquinolin-5-yl)ethyl)amino)picolinic acid Enantiomer 2 (14.4 mg, 5% yield, >99% ee), ¹H-NMR (300 MHz, Methanol-d4) δ 7.98 (s, 1H), 7.52 (d, J=1.8 Hz, 1H), 7.13 (d, J=8.8 Hz, 1H), 6.83 (d, J=8.9 Hz, 1H), 6.48 (s, 1H), 5.05-5.15 (m, 1H), 3.61 (s, 3H), 3.10-2.81 (m, 4H), 2.37 (s, 3H), 1.66 (d, J=6.7 Hz, 7H), 1.06 (s, 6H); MS: (ES⁺) m/z=483.1 [M+H]⁺.

### Example 5: 2-((1-(3-(4,4-dimethylcyclohex-1-en-1-yl)-2,7-dimethyl-1-oxo-1,2-dihydroisoquinolin-5-yl)ethyl)amino)benzoic acid (Enantiomer 1)

### Example 6: 2-((1-(3-(4,4-dimethylcyclohex-1-en-1-yl)-2,7-dimethyl-1-oxo-1,2-dihydroisoquinolin-5-yl)ethyl)amino)benzoic acid (Enantiomer 2)

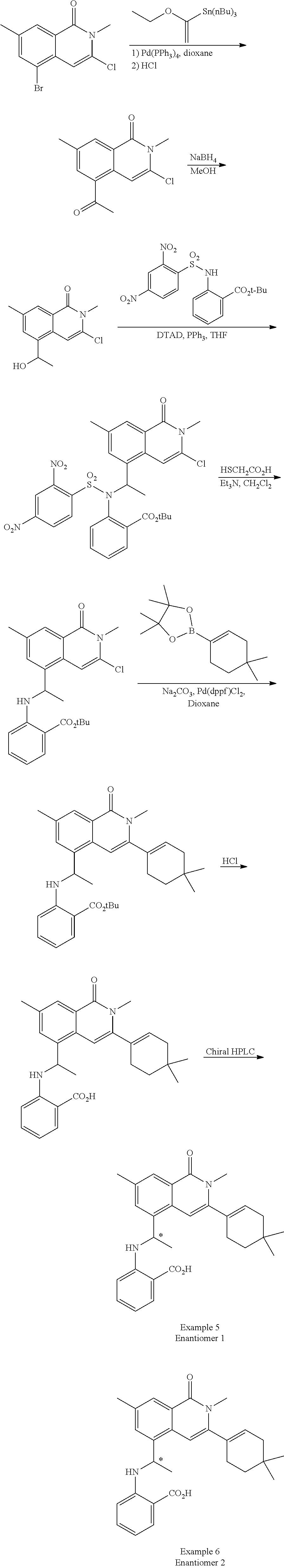

### Step 1: Preparation of 5-acetyl-3-chloro-2,7-dimethylisoquinolin-1-one

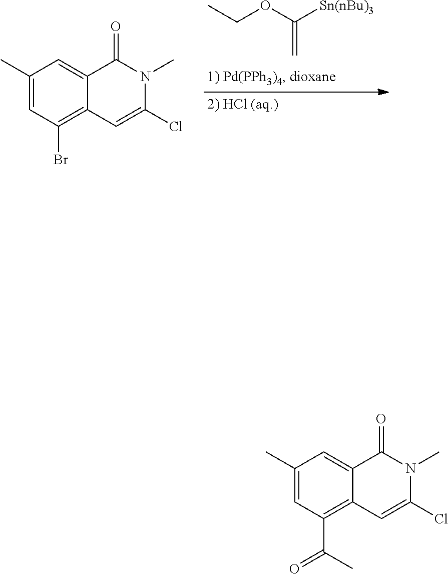

A mixture of 5-bromo-3-chloro-2,7-dimethylisoquinolin-1-one (2.6 g, 9.07 mmol), tributyl(1-ethoxyethenyl)stannane (3.60 g, 9.98 mmol) and Pd(PPh₃)₄ (1.05 g, 0.91 mmol) in anhydrous 1,4-dioxane (25 mL) was stirred overnight at 100° C. under a nitrogen atmosphere. The reaction mixture was cooled to room temperature, treated with aqueous 1 N HCl (10 mL), and stirred for 15 min. The resulting mixture was diluted with water (60 mL) and extracted with ethyl acetate (3×100 mL). The combined organic layers were washed with water (3×60 mL) and brine (50 mL), dried over anhydrous sodium sulfate, and concentrated under reduced pressure to afford 5-acetyl-3-chloro-2,7-dimethylisoquinolin-1-one (1.9 g, 83%) as a yellow solid. MS: ES⁺ (m/z)=250.1 [M+H]⁺.

### Step 2: Preparation of 3-chloro-5-(1-hydroxyethyl)-2,7-dimethylisoquinolin-1-one

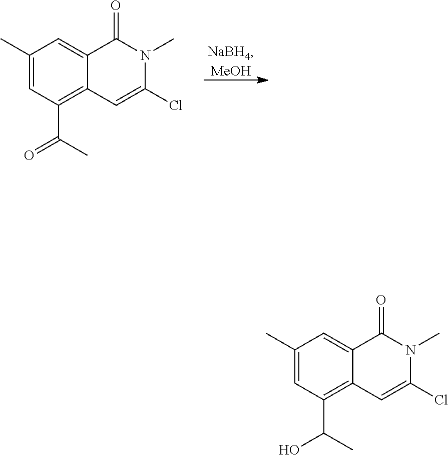

To a stirred solution of 5-acetyl-3-chloro-2,7-dimethylisoquinolin-1-one (1.9 g, 7.61 mmol) in MeOH (20 mL) was slowly added NaBH₄ (0.58 g, 15.218 mmol) in portions at 0° C. The resulting mixture was stirred for 2 h at room temperature. The reaction was quenched with water (30 mL) and extracted with EtOAc (3×80 mL). The combined organic layers were washed with brine (2×60 mL), dried over anhydrous Na₂SO₄, and then concentrated under reduced pressure. The residue was purified by silica gel column chromatography (PE/EA=1:1) to afford 3-chloro-5-(1-hydroxyethyl)-2,7-dimethylisoquinolin-1-one (1.5 g, 78%) as an off-white solid. MS: (ES⁺) m/z=252.1 [M+H]⁺.

### Step 3: Preparation of tert-butyl 2-((N-(1-(3-chloro-2,7-dimethyl-1-oxo-1,2-dihydroisoquinolin-5-yl)ethyl)-2,4-dinitrophenyl)sulfonamido)benzoate

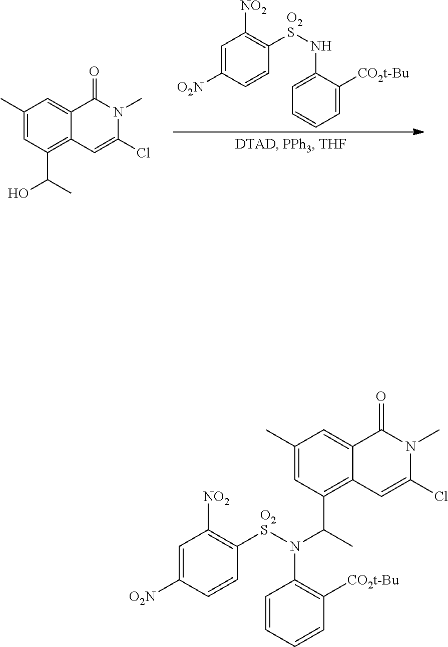 [SMILES: Cc1cc(C(C)O)c2cc(Cl)n(C)c(=O)c2c1 ; CC(C)(C)OC(=O)c1ccccc1NS(=O)(=O)c1ccc([N+](=O)[O-])cc1[N+](=O)[O-] ; Cc1cc(C(C)N(c2ccccc2C(=O)OC(C)(C)C)S(=O)(=O)c2ccc([N+](=O)[O-])cc2[N+](=O)[O-])c2cc(Cl)n(C)c(=O)c2c1]

To a solution of 3-chloro-5-(1-hydroxyethyl)-2,7-dimethylisoquinolin-1-one (1.4 g, 5.56 mmol), tert-butyl 2-(2,4-dinitrobenzenesulfonamido)benzoate (2.59 g, 6.12 mmol) and PPh₃ (3.65 g, 13.91 mmol) in THF (20 mL) was added a solution of (E)-N-[[(tert-butoxy)carbonyl]imino](tert-butoxy) formamide (3.84 g, 16.69 mmol) in tetrahydrofuran (1.5 mL) at 0° C. under a nitrogen atmosphere. The resulting solution was stirred overnight at room temperature. The residue was concentrated under reduced pressure and purified by silica gel column chromatography (PE/EA=3:1) to afford tert-butyl 2-((N-(1-(3-chloro-2,7-dimethyl-1-oxo-1,2-dihydroisoquinolin-5-yl)ethyl)-2,4-dinitrophenyl)sulfonamido)benzoate (2.4 g, 65%) as a yellow solid. MS: (ES⁺) m/z=657.3 [M+H]⁺.

### Step 4: Preparation of tert-butyl 2-{[1-(3-chloro-2,7-dimethyl-1-oxoisoquinolin-5-yl)ethyl]amino}benzoate

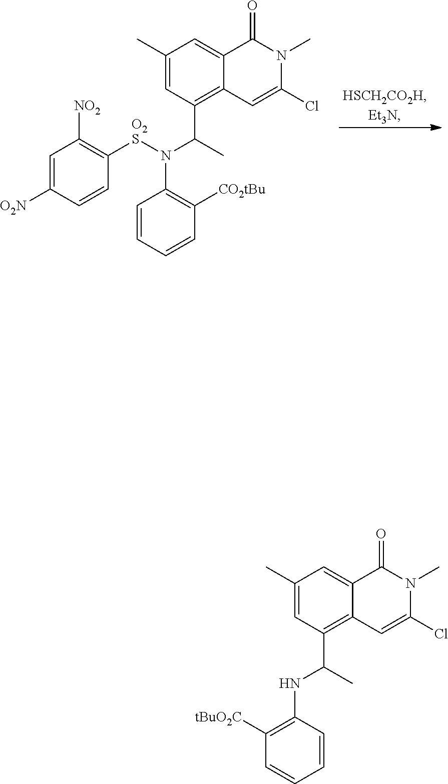 [SMILES: Cc1cc(C(C)N(c2ccccc2C(=O)OC(C)(C)C)S(=O)(=O)c2ccc([N+](=O)[O-])cc2[N+](=O)[O-])c2cc(Cl)n(C)c(=O)c2c1 ; "HSCH2CO2H,Et3N," ; Cc1cc(C(C)Nc2ccccc2C(=O)OC(C)(C)C)c2cc(Cl)n(C)c(=O)c2c1]

To a stirred solution of tert-butyl 2-((N-(1-(3-chloro-2,7-dimethyl-1-oxo-1,2-dihydroisoquinolin-5-yl)ethyl)-2,4-dinitrophenyl)sulfonamido)benzoate (2.4 g, 3.65 mmol) in DCM (12 mL) was slowly added Et₃N (0.74 g, 7.30 mmol) and 2-sulfanylacetic acid (0.50 g, 5.48 mmol) dropwise at 0° C. The resulting mixture was stirred for 3 h at rt. The reaction was quenched with water (30 mL) and extracted with DCM (3×50 mL). The combined organic layers were washed with brine (2×30 mL), dried over anhydrous Na₂SO₄, and then concentrated under reduced pressure. The residue was purified by prep TLC (PE/EA=6:1) to afford tert-butyl 2-((1-(3-chloro-2,7-dimethyl-1-oxo-1,2-dihydroisoquinolin-5-yl)ethyl)amino)benzoate (1.2 g, 76%) as a light yellow solid. MS: (ES⁺) m/z=427.1 [M+H]⁺.

### Step 5: Preparation of tert-butyl 2-((1-(3-(4,4-dimethylcyclohex-1-en-1-yl)-2,7-dimethyl-1-oxo-1,2-dihydroisoquinolin-5-yl)ethyl)amino)benzoate

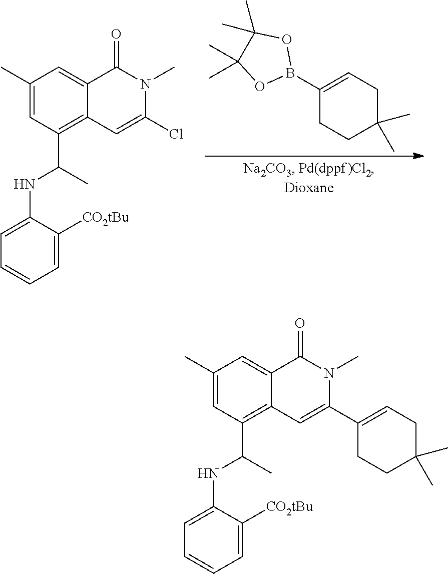

A mixture of tert-butyl 2-((1-(3-chloro-2,7-dimethyl-1-oxo-1,2-dihydroisoquinolin-5-yl)ethyl)amino)benzoate (200 mg, 0.47 mmol), 2-(4,4-dimethylcyclohex-1-en-1-yl)-4,4,5,5-tetramethyl-1,3,2-dioxaborolane (221 mg, 0.94 mmol), Na₂CO₃ (149 mg, 1.40 mmol), and Pd(dppf)Cl₂ (38 mg, 0.047 mmol) in 1,4-dioxane (10 mL) and H₂O (2 mL) was stirred overnight at 100° C. under a nitrogen atmosphere. The reaction mixture was cooled to room temperature. The residue was concentrated under reduced pressure and purified by prep TLC (PE/EA=3:1) to afford tert-butyl 2-((1-(3-(4,4-dimethylcyclohex-1-en-1-yl)-2,7-dimethyl-1-oxo-1,2-dihydroisoquinolin-5-yl)ethyl)amino)benzoate (140 mg, 59%) as a light yellow solid. MS: (ES⁺) m/z=501.3 [M+H]⁺.

### Step 6: Preparation of 2-((1-(3-(4,4-dimethylcyclohex-1-en-1-yl)-2,7-dimethyl-1-oxo-1,2-dihydroisoquinolin-5-yl)ethyl)amino)benzoic acid

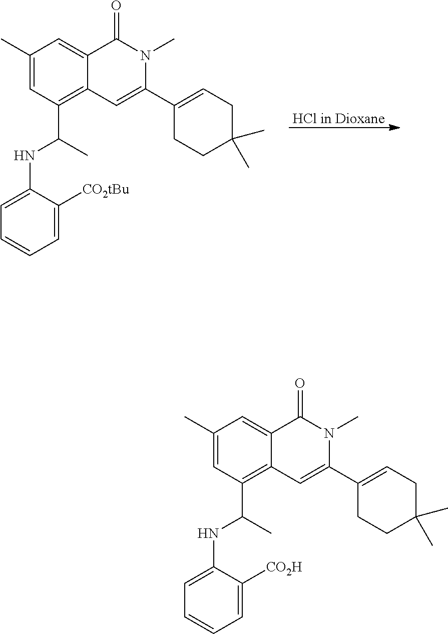

A stirred solution of tert-butyl 2-((1-(3-(4,4-dimethylcyclohex-1-en-1-yl)-2,7-dimethyl-1-oxo-1,2-dihydroisoquinolin-5-yl)ethyl)amino)benzoate (120 mg, 0.24 mmol) in 4 M HCl in 1,4-dioxane (10 mL) was stirred overnight at room temperature and then concentrated under reduced pressure. The residue was purified by prep TLC (DCM/MeOH=25:1) to afford 2-((1-(3-(4,4-dimethylcyclohex-1-en-1-yl)-2,7-dimethyl-1-oxo-1,2-dihydroisoquinolin-5-yl)ethyl)amino)benzoic acid (80 mg, 75%) as a yellow solid. MS: (ES⁺) m/z=445.3 [M+H]⁺.

### Step 7: Preparation of 2-((1-(3-(4,4-dimethylcyclohex-1-en-1-yl)-2,7-dimethyl-1-oxo-1,2-dihydroisoquinolin-5-yl)ethyl)amino)benzoic acid (Enantiomers 1 and 2)

A racemic mixture of 2-((1-(3-(4,4-dimethylcyclohex-1-en-1-yl)-2,7-dimethyl-1-oxo-1,2-dihydroisoquinolin-5-yl)ethyl)amino)benzoic acid (80 mg, 0.18 mmol) was separated by chiral HPLC (Column: CHIRALPAK AD-H, 2*25 cm, 5 μm; Mobile Phase A: Hex (0.1% FA)-HPLC, Mobile Phase B: EtOH-HPLC; Flow rate: 20 mL/min; Gradient: 10% B to 10% B in 12 min; Wavelengths: 220/254 nm; RT1(min): 6.58; RT2(min): 10.85; Sample Solvent: EtOH-HPLC; Injection Volume: 0.5 mL) to afford each enantiomer of 2-((1-(3-(4,4-dimethylcyclohex-1-en-1-yl)-2,7-dimethyl-1-oxo-1,2-dihydroisoquinolin-5-yl)ethyl)amino)benzoic acid as a light yellow solid:

2-((1-(3-(4,4-dimethylcyclohex-1-en-1-yl)-2,7-dimethyl-1-oxo-1,2-dihydroisoquinolin-5-yl)ethyl)amino)benzoic acid Enantiomer 1 (29.3 mg, 36%, 99.9% ee). MS: (ES⁺) m/z=445.1 [M+H]⁺. ¹H NMR: (300 MHz, Methanol-d4) δ 8.01 (s, 1H), 7.92-7.88 (m, 1H), 7.55 (d, J=1.9 Hz, 1H), 7.11-7.10 (m, 1H), 6.75 (s, 1H), 6.51-6.50 (m, 1H), 6.36-6.27 (m, 1H), 5.96-5.87 (m, 1H), 5.20-5.10 (m, 1H), 3.54 (s, 3H), 2.39 (s, 3H), 2.36-2.25 (m, 2H), 2.10-2.02 (m, 2H), 1.65-1.53 (m, 5H), 1.04 (d, J=1.1 Hz, 6H).

2-((1-(3-(4,4-dimethylcyclohex-1-en-1-yl)-2,7-dimethyl-1-oxo-1,2-dihydroisoquinolin-5-yl)ethyl)amino)benzoic acid Enantiomer 2 (29.1 mg, 37%, 99.9% ee). MS: (ES⁺) m/z=445.1 [M+H]⁺. ¹H NMR: (300 MHz, Methanol-d4) δ 8.02 (s, 1H), 7.92-7.88 (m, 1H), 7.55 (d, J=1.8 Hz, 1H), 7.12-7.11 (m, 1H), 6.75 (s, 1H), 6.57-6.45 (m, 1H), 6.36-6.27 (m, 1H), 5.96-5.87 (m, 1H), 5.20-5.10 (m, 1H), 3.53 (s, 3H), 2.39 (s, 3H), 2.36-2.25 (m, 2H), 2.10-2.02 (m, 2H), 1.65-1.52 (m, 5H), 1.03 (d, J=1.3 Hz, 6H).

### Example 7: 2-((1-(3-(5-fluoro-1H-indol-2-yl)-2,7-dimethyl-1-oxo-1,2-dihydroisoquinolin-5-yl)ethyl)amino)benzoic acid

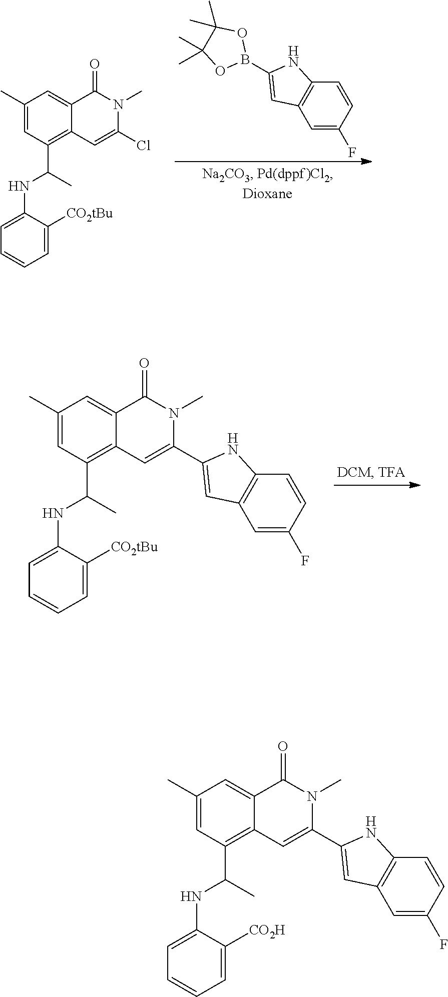

### Step 1: Preparation of tert-butyl 2-((1-(3-(5-fluoro-1H-indol-2-yl)-2,7-dimethyl-1-oxo-1,2-dihydroisoquinolin-5-yl)ethyl)amino)benzoate

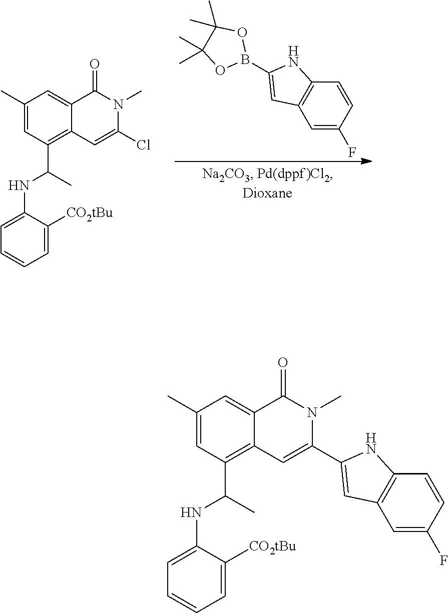 [SMILES: Cc1cc(C(C)Nc2ccccc2C(=O)OC(C)(C)C)c2cc(Cl)n(C)c(=O)c2c1 ; CC1(C)OB(c2cc3cc(F)ccc3[nH]2)OC1(C)C ; Cc1cc(C(C)Nc2ccccc2C(=O)OC(C)(C)C)c2cc(-c3cc4cc(F)ccc4[nH]3)n(C)c(=O)c2c1]

A mixture of tert-butyl 2-((1-(3-chloro-2,7-dimethyl-1-oxo-1,2-dihydroisoquinolin-5-yl)ethyl)amino)benzoate (450 mg, 1.05 mmol), 5-fluoro-2-(4,4,5,5-tetramethyl-1,3,2-dioxaborolan-2-yl)-1H-indole (330 mg, 1.27 mmol), Na₂CO₃ (335 mg, 3.16 mmol), Pd(dppf)Cl₂ (129 mg, 0.16 mmol) in 1,4-dioxane (8 mL) and H₂O (1 mL) was stirred overnight at 100° C. under a nitrogen atmosphere. The reaction mixture was cooled to room temperature. The solid was filtrated and washed with DCM (2×50 mL). The filtrate was concentrated under reduced pressure and purified by prep TLC (PE/EA=3:1) to afford tert-butyl 2-((1-(3-(5-fluoro-1H-indol-2-yl)-2,7-dimethyl-1-oxo-1,2-dihydroisoquinolin-5-yl)ethyl)amino)benzoate (300 mg, 54%) as a yellow solid. MS: (ES⁺) m/z=526.2 [M+H]⁺.

### Step 2: Preparation of 2-((1-(3-(5-fluoro-1H-indol-2-yl)-2,7-dimethyl-1-oxo-1,2-dihydroisoquinolin-5-yl)ethyl)amino)benzoic acid

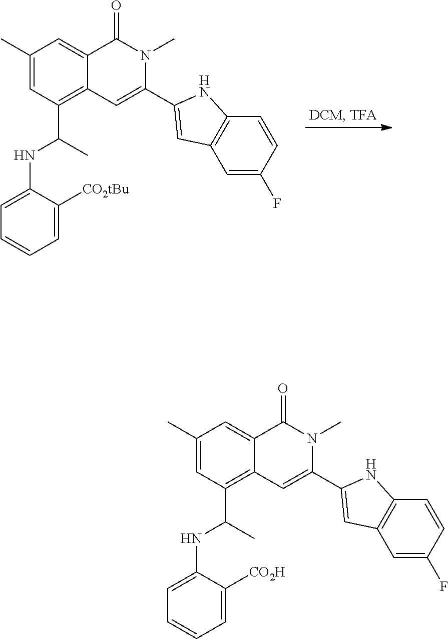 [SMILES: Cc1cc(C(C)Nc2ccccc2C(=O)OC(C)(C)C)c2cc(-c3cc4cc(F)ccc4[nH]3)n(C)c(=O)c2c1 ; Cc1cc(C(C)Nc2ccccc2C(=O)O)c2cc(-c3cc4cc(F)ccc4[nH]3)n(C)c(=O)c2c1]

A solution of tert-butyl 2-((1-(3-(5-fluoro-1H-indol-2-yl)-2,7-dimethyl-1-oxo-1,2-dihydroisoquinolin-5-yl)ethyl)amino)benzoate (100 mg, 0.19 mmol) in TFA (6 mL) and DCM (6 mL) was stirred overnight at room temperature. The resulting mixture was concentrated under reduced pressure and purified by prep TLC (CH₂Cl₂/MeOH=15:1). The desired product was purified further by reverse flash chromatography (column, C18 silica gel; mobile phase, ACN in H₂O, 30% to 50% gradient in 10 min; detector, UV 254 nm). Finally, the desired product was purified further by prep HPLC (Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 8% B to 38% B in 9 min, 38% B; Wavelengths: 254 nm) to afford 2-((1-(3-(5-fluoro-1H-indol-2-yl)-2,7-dimethyl-1-oxo-1,2-dihydroisoquinolin-5-yl)ethyl)amino)benzoic acid (18.1 mg, 20%) as a white solid. MS: (ES⁺) m/z=470.1 [M+H]⁺. ¹H NMR: (400 MHz, DMSO-d6) δ 11.87 (s, 1H), 8.76 (s, 1H), 8.00 (s, 1H), 7.83 (d, J=7.9 Hz, 1H), 7.56-7.44 (m, 2H), 7.42-7.36 (m, 1H), 7.21-7.00 (m, 3H), 6.84 (d, J=2.0 Hz, 1H), 6.50-6.46 (m, 1H), 6.32 (d, J=8.5 Hz, 1H), 5.31-5.25 (m, 1H), 3.56 (s, 3H), 2.38 (s, 3H), 1.53 (d, J=6.6 Hz, 3H).

### Intermediate 1

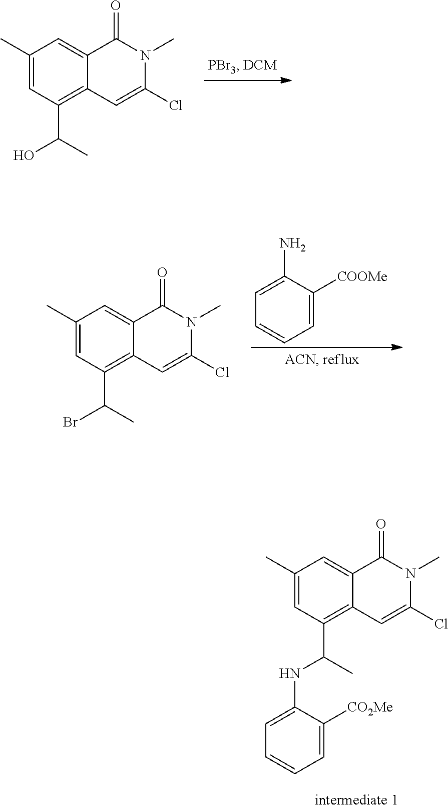

### Step 1: Preparation of 5-(1-bromoethyl)-3-chloro-2,7-dimethylisoquinolin-1 (2H)-one

To a stirred solution of 3-chloro-5-(1-hydroxyethyl)-2,7-dimethylisoquinolin-1-one (1 g, 3.97 mmol) in DCM (12 mL) was added PBr₃ (2.15 g, 7.95 mmol) at 0° C. The resulting mixture was stirred for 4 h at room temperature under a nitrogen atmosphere. The reaction was quenched with H₂O (30 mL) at 0° C. and the mixture was adjusted to approximately pH 7 with saturated aqueous NaHCO₃. The resulting mixture was extracted with DCM (3×20 mL). The combined organic layers were washed with brine (2×20 mL), dried over anhydrous Na₂SO₄, and then concentrated under reduced pressure to yield 5-(1-bromoethyl)-3-chloro-2,7-dimethylisoquinolin-1 (2H)-one which was used in the next step without further purification. MS: (ES⁺) m/z=314.0 [M+H]⁺.

### Step 2: Preparation of methyl 2-((1-(3-chloro-2,7-dimethyl-1-oxo-1,2-dihydroisoquinolin-5-yl)ethyl)amino)benzoate

To a stirred solution of 5-(1-bromoethyl)-3-chloro-2,7-dimethylisoquinolin-1 (2H)-one (1 g, 3.18 mmol) in ACN (30 mL) and THF (10 mL) was added methyl anthranilate (1.20 g, 7.95 mmol). The resulting mixture was stirred for 3 h at 80° C. under a nitrogen atmosphere and then concentrated under reduced pressure. The residue was purified by silica gel column chromatography (PE/EA=4:1) to afford methyl 2-((1-(3-chloro-2,7-dimethyl-1-oxo-1,2-dihydroisoquinolin-5-yl)ethyl)amino)benzoate (0.8 g, 65%) as a light yellow solid. MS: (ES⁺) m/z=385.2 [M+H]⁺.

### Example 8: 2-((1-(2,7-dimethyl-1-oxo-3-(4-(2,2,2-trifluoroethyl)piperazin-1-yl)-1,2-dihydroisoquinolin-5-yl)ethyl)amino)benzoic acid (Enantiomer 1)

### Example 9: 2-((1-(2,7-dimethyl-1-oxo-3-(4-(2,2,2-trifluoroethyl)piperazin-1-yl)-1,2-dihydroisoquinolin-5-yl)ethyl)amino)benzoic acid (Enantiomer 2)

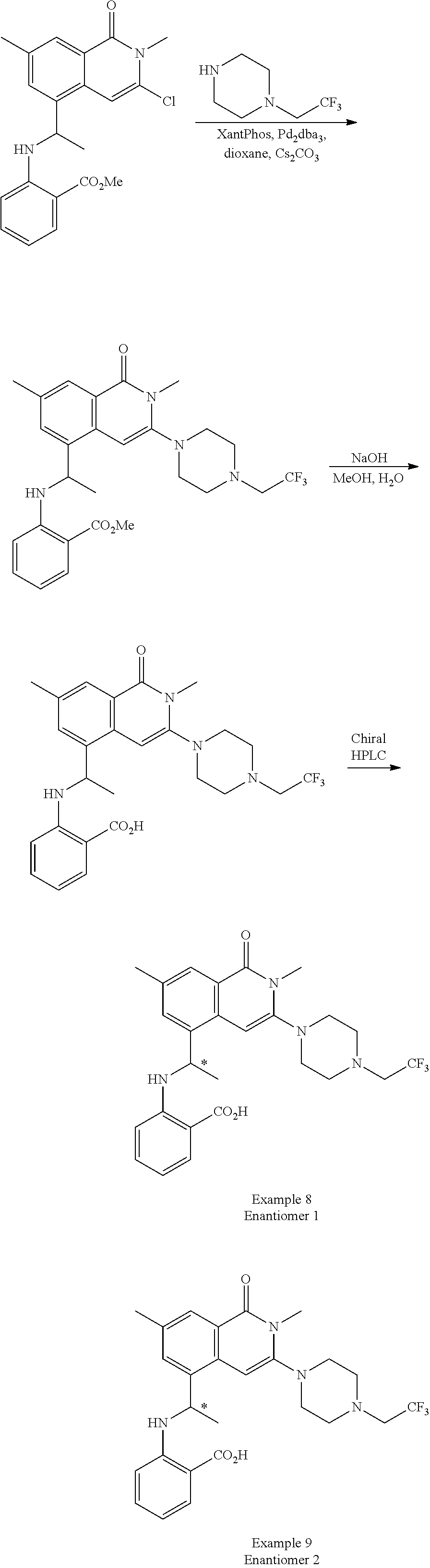

### Step 1: Preparation of methyl 2-((1-(2,7-dimethyl-1-oxo-3-(4-(2,2,2-trifluoroethyl)piperazin-1-yl)-1,2-dihydroisoquinolin-5-yl)ethyl)amino)benzoate

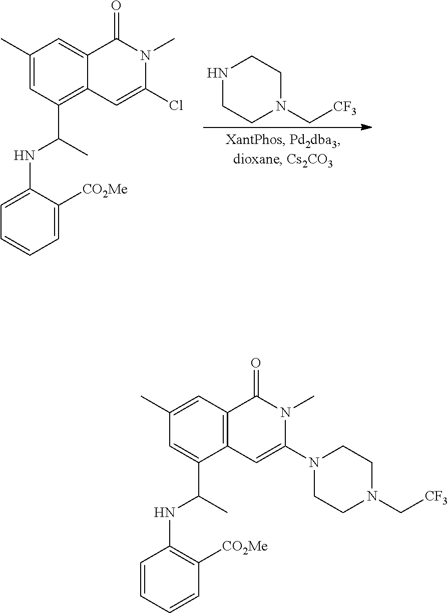 [SMILES: COC(=O)c1ccccc1NC(C)c1cc(C)cc2c(=O)n(C)c(Cl)cc12 ; FC(F)(F)CN1CCNCC1 ; COC(=O)c1ccccc1NC(C)c1cc(C)cc2c(=O)n(C)c(N3CCN(CC(F)(F)F)CC3)cc12]

To a stirred solution of methyl 2-((1-(3-chloro-2,7-dimethyl-1-oxo-1,2-dihydroisoquinolin-5-yl)ethyl)amino)benzoate (200 mg, 0.52 mmol) and 1-(2,2,2-trifluoroethyl) piperazine dihydrochloride (187 mg, 0.78 mmol), Cs₂CO₃ (508 mg, 1.56 mmol) in dioxane (3 mL) were added Pd₂(dba)₃ (95 mg, 0.10 mmol) and RuPhos (60 mg, 0.10 mmol) in portions at room temperature under a nitrogen atmosphere. The resulting mixture was stirred overnight at 100° C. under a nitrogen atmosphere. The resulting mixture was quenched with 30 ml H₂O. The mixture solution was extracted with EtOAc (3×30 mL). The combined organic layers were washed with brine (3×20 mL), dried over anhydrous Na₂SO₄, and then were concentrated under reduced pressure. The residue was purified by silica gel column chromatography (PE/EA=1:1) to afford methyl 2-((1-(2,7-dimethyl-1-oxo-3-(4-(2,2,2-trifluoroethyl)piperazin-1-yl)-1,2-dihydroisoquinolin-5-yl)ethyl)amino)benzoate (120 mg, 44%) as a yellow oil. MS: (ES⁺) m/z=517.6 [M+H]⁺.

### Step 2: Preparation of 2-((1-(2,7-dimethyl-1-oxo-3-(4-(2,2,2-trifluoroethyl)piperazin-1-yl)-1,2-dihydroisoquinolin-5-yl)ethyl)amino)benzoic acid

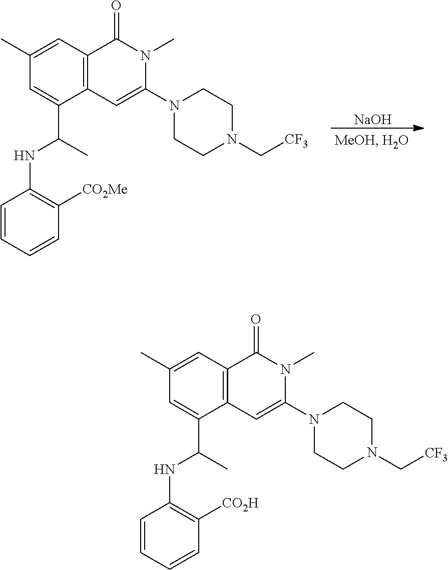 [SMILES: COC(=O)c1ccccc1NC(C)c1cc(C)cc2c(=O)n(C)c(N3CCN(CC(F)(F)F)CC3)cc12 ; "NaOH" ; Cc1cc(C(C)Nc2ccccc2C(=O)O)c2cc(N3CCN(CC(F)(F)F)CC3)n(C)c(=O)c2c1]

A mixture of methyl 2-((1-(2,7-dimethyl-1-oxo-3-(4-(2,2,2-trifluoroethyl)piperazin-1-yl)-1,2-dihydroisoquinolin-5-yl)ethyl)amino)benzoate (30 mg, 0.058 mmol), MeOH (600 μL), H₂O (120 μL) and NaOH (4.65 mg, 0.12 mmol) was stirred overnight at 50° C. The mixture was acidified to pH 5 with an aqueous NaH₂PO₄ solution. The resulting mixture was diluted with H₂O (20 mL) and extracted with EtOAc (3×20 mL). The combined organic layers were washed with brine (3×20 mL), dried over anhydrous Na₂SO₄, and then concentrated under reduced pressure. The residue was purified by reverse phase flash chromatography (Column, C18 silica gel; mobile phase, MeCN in water (0.1% FA), 5% to 100% gradient in 40 min; detector, UV 254 nm) to afford 2-((1-(2,7-dimethyl-1-oxo-3-(4-(2,2,2-trifluoroethyl)piperazin-1-yl)-1,2-dihydroisoquinolin-5-yl)ethyl)amino)benzoic acid (14.4 mg, 49%) as an off-white solid. MS: (ES⁺) m/z=503.5 [M+H]⁺.

### Step 3: Preparation of 2-((1-(2,7-dimethyl-1-oxo-3-(4-(2,2,2-trifluoroethyl)piperazin-1-yl)-1,2-dihydroisoquinolin-5-yl)ethyl)amino)benzoic acid (Enantiomers 1 and 2)

A racemic mixture of 2-((1-(2,7-dimethyl-1-oxo-3-(4-(2,2,2-trifluoroethyl)piperazin-1-yl)-1,2-dihydroisoquinolin-5-yl)ethyl)amino)benzoic acid (60 mg, 0.119 mmol) was separated by prep chiral HPLC (Column: CHIRALPAK IC-3, 4.6*50 mm, 3 μm; Mobile Phase: Hex (0.1% FA): IPA=80:20; Flow rate: 1 mL/min) to yield each enantiomer of 2-((1-(2,7-dimethyl-1-oxo-3-(4-(2,2,2-trifluoroethyl)piperazin-1-yl)-1,2-dihydroisoquinolin-5-yl)ethyl)amino)benzoic acid as a light yellow solid:

2-((1-(2,7-dimethyl-1-oxo-3-(4-(2,2,2-trifluoroethyl)piperazin-1-yl)-1,2-dihydroisoquinolin-5-yl)ethyl)amino)benzoic acid Enantiomer 1 (21.2 mg, 35%, >99.9% ee). MS: (ES⁺) m/z=503.5 [M+H]⁺. ¹H NMR: (300 MHz, DMSO-d6) δ 12.86 (s, 1H), 8.46 (s, 1H), 7.87 (s, 1H), 7.86-7.80 (m, 1H), 7.41 (d, J=1.9 Hz, 1H), 7.20-7.17 (m, 1H), 6.57-6.45 (m, 1H), 6.44-6.32 (m, 2H), 5.22-5.15 (m, 1H), 3.49 (s, 3H), 3.50-3.34 (m, 2H), 3.21-2.61 (m, 8H), 2.32 (s, 3H), 1.53 (d, J=6.5 Hz, 3H).

2-((1-(2,7-dimethyl-1-oxo-3-(4-(2,2,2-trifluoroethyl)piperazin-1-yl)-1,2-dihydroisoquinolin-5-yl)ethyl)amino)benzoic acid Enantiomer 2 (24.0 mg, 39%, >99.9% ee). MS: (ES⁺) m/z=503.5 [M+H]⁺. ¹HNMR: (300 MHz, DMSO-d6) δ 12.76 (s, 1H), 8.43 (s, 1H), 7.87 (s, 1H), 7.86-7.80 (m, 1H), 7.41 (d, J=1.9 Hz, 1H), 7.20-7.18 (m, 1H), 6.57-6.45 (m, 1H), 6.44-6.32 (m, 2H), 5.22-5.15 (m, 1H), 3.49 (s, 3H), 3.50-3.31 (m, 2H), 3.21-2.60 (m, 8H), 2.32 (s, 3H), 1.53 (d, J=6.5 Hz, 3H).

### Example 10: 2-((1-(3-(3-azabicyclo[3.2.1]octan-3-yl)-2,7-dimethyl-1-oxo-1,2-dihydroisoquinolin-5-yl)ethyl)amino)benzoic acid (Enantiomer 1)

### Example 11: 2-((1-(3-(3-azabicyclo[3.2.1]octan-3-yl)-2,7-dimethyl-1-oxo-1,2-dihydroisoquinolin-5-yl)ethyl)amino)benzoic acid (Enantiomer 2)

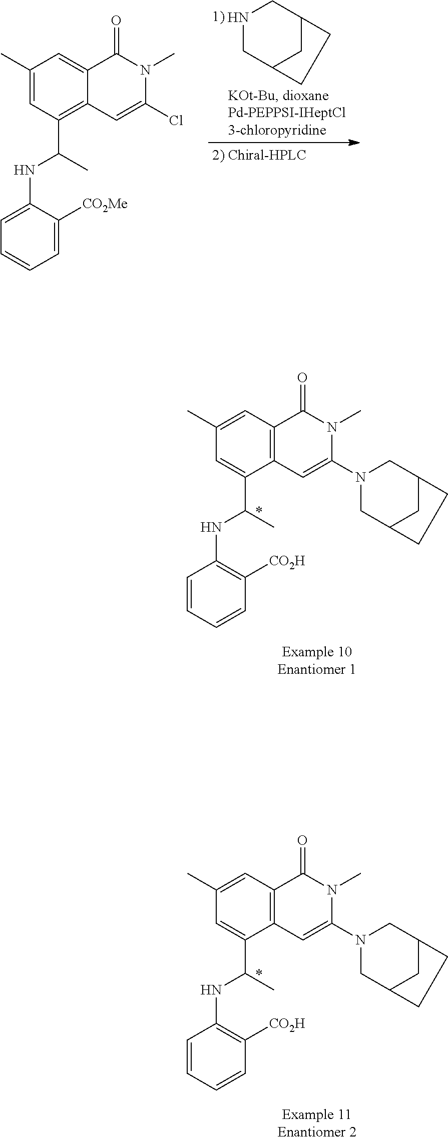 [SMILES: COC(=O)c1ccccc1NC(C)c1cc(C)cc2c(=O)n(C)c(Cl)cc12 ; C1CC2CNCC1C2 ; Cc1cc(C(C)Nc2ccccc2C(=O)O)c2cc(N3CC4CCC(C4)C3)n(C)c(=O)c2c1 ; Cc1cc(C(C)Nc2ccccc2C(=O)O)c2cc(N3CC4CCC(C4)C3)n(C)c(=O)c2c1]

A mixture of methyl 2-((1-(3-chloro-2,7-dimethyl-1-oxo-1,2-dihydroisoquinolin-5-yl)ethyl)amino)benzoate (300 mg, 0.78 mmol), 3-azabicyclo[3.2.1]octane hydrochloride (288 mg, 1.95 mmol), potassium 2-methylpropan-2-olate (350 mg, 3.12 mmol) and Pd-PEPPSI-IHeptCI 3-chloropyridine (379 mg, 0.39 mmol) in anhydrous dioxane (20 mL) was stirred overnight at 100° C. under a nitrogen atmosphere. The reaction mixture was cooled to room temperature. The solid was filtrated and washed with DCM (2×50 mL). The filtrate was concentrated under reduced pressure. The residue was purified by prep TLC (DCM/MeOH=10:1) to afford 2-((1-(3-(3-azabicyclo[3.2.1]octan-3-yl)-2,7-dimethyl-1-oxo-1,2-dihydroisoquinolin-5-yl)ethyl)amino)benzoic acid (80 mg, 23%) as a yellow solid. MS: (ES⁺) m/z=446.6 [M+H]⁺.

A racemic mixture of 2-((1-(3-(3-azabicyclo[3.2.1]octan-3-yl)-2,7-dimethyl-1-oxo-1,2-dihydroisoquinolin-5-yl)ethyl)amino)benzoic acid (80 mg, 0.18 mmol) was separated by prep chiral HPLC (Column: CHIRALCEL OD-3, 4.6*50 mm, 3 μm; Mobile Phase Hex (0.1% FA):EtOH=80:20; Flow rate: 1 mL/min) to yield each enantiomer of 2-((1-(3-(3-azabicyclo[3.2.1]octan-3-yl)-2,7-dimethyl-1-oxo-1,2-dihydroisoquinolin-5-yl)ethyl)amino)benzoic acid as a white solid:

2-((1-(3-(3-azabicyclo[3.2.1]octan-3-yl)-2,7-dimethyl-1-oxo-1,2-dihydroisoquinolin-5-yl)ethyl)amino)benzoic acid Enantiomer 1 (4.8 mg, 6%, >99% ee). MS: (ES⁺) m/z=446.6 [M+H]⁺. ¹H NMR: (300 MHz, DMSO-d6) δ 12.74 (s, 1H), 8.35 (d, J=5.9 Hz, 1H), 7.87 (d, J=1.8 Hz, 1H), 7.80 (s, 1H), 7.41 (d, J=1.9 Hz, 1H), 7.25-7.20 (m, 1H), 6.58-6.47 (m, 1H), 6.44 (s, 1H), 6.37 (d, J=8.3 Hz, 1H), 5.20-5.12 (m, 1H), 3.56 (s, 3H), 3.02-2.95 (m 2H), 2.80-2.76 (m, 2H), 2.32-2.28 (m, 5H), 1.82 (d, J=7.7 Hz, 2H), 1.77-1.62 (m, 2H), 1.53 (d, J=6.7 Hz, 3H).

2-((1-(3-(3-azabicyclo[3.2.1]octan-3-yl)-2,7-dimethyl-1-oxo-1,2-dihydroisoquinolin-5-yl)ethyl)amino)benzoic acid Enantiomer 2 (4.7 mg, 6%, >99% ee). MS: (ES⁺) m/z=446.6 [M+H]⁺. ¹H NMR: (300 MHz, DMSO-d6) δ 12.74 (s, 1H), 8.35 (d, J=5.9 Hz, 1H), 7.87 (d, J=1.8 Hz, 1H), 7.80 (s, 1H), 7.41 (d, J=1.9 Hz, 1H), 7.25-7.20 (m, 1H), 6.58-6.47 (m, 1H), 6.44 (s, 1H), 6.37 (d, J=8.3 Hz, 1H), 5.20-5.12 (m, 1H), 3.56 (s, 3H), 3.02-2.95 (m 2H), 2.80-2.76 (m, 2H), 2.32-2.28 (m, 5H), 1.82 (d, J=7.7 Hz, 2H), 1.77-1.62 (m, 2H), 1.53 (d, J=6.7 Hz, 3H).

### Example 12: 2-((1-(2,7-dimethyl-3-(4-methylpiperazin-1-yl)-1-oxo-1,2-dihydroisoquinolin-5-yl)ethyl)amino)benzoic acid (Enantiomer 1)

### Example 13: 2-((1-(2,7-dimethyl-3-(4-methylpiperazin-1-yl)-1-oxo-1,2-dihydroisoquinolin-5-yl)ethyl)amino)benzoic acid (Enantiomer 2)

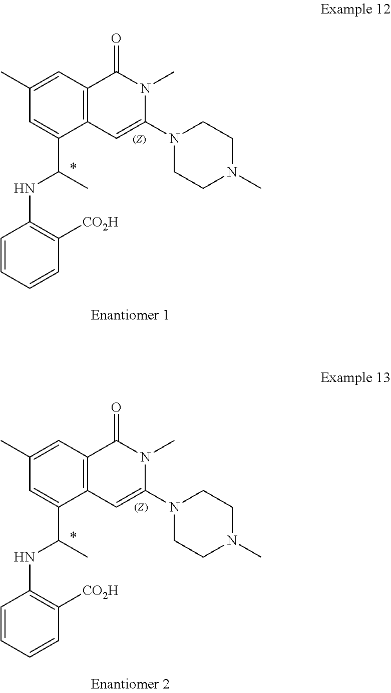

Prepared in a manner similar to Examples 8 and 9 using 1-methylpiperazine in place of 1-(2,2,2-trifluoroethyl)piperazine dihydrochloride in Step 1:

2-((1-(2,7-dimethyl-3-(4-methylpiperazin-1-yl)-1-oxo-1,2-dihydroisoquinolin-5-yl)ethyl)amino)benzoic acid Enantiomer 1 (>99.9% ee). MS: (ES⁺) m/z=435.5 [M+H]⁺. ¹H NMR: (300 MHz, DMSO-d6) δ 8.46 (s, 1H), 7.87 (s, 1H), 7.80 (s, 1H), 7.42 (d, J=1.9 Hz, 1H), 7.24-7.12 (m, 1H), 6.61-6.51 (m, 1H), 6.43-6.32 (m, 2H), 5.20-5.12 (m, 1H), 4.20-3.91 (m, 4H), 3.48 (s, 3H), 3.10-2.95 (m, 4H), 2.29 (s, 3H), 2.26 (s, 3H), 1.54 (d, J=6.5 Hz, 3H).

2-((1-(2,7-dimethyl-3-(4-methylpiperazin-1-yl)-1-oxo-1,2-dihydroisoquinolin-5-yl)ethyl)amino)benzoic acid Enantiomer 2 (>99.9% ee). MS: (ES⁺) m/z=435.5 [M+H]⁺. ¹H NMR: (300 MHz, DMSO-d6) δ 8.46 (s, 1H), 7.87 (s, 1H), 7.80 (s, 1H), 7.42 (d, J=1.9 Hz, 1H), 7.24-7.12 (m, 1H), 6.61-6.51 (m, 1H), 6.43-6.32 (m, 2H), 5.20-5.12 (m, 1H), 4.20-3.91 (m, 4H), 3.48 (s, 3H), 3.10-2.95 (m, 4H), 2.29 (s, 3H), 2.26 (s, 3H), 1.54 (d, J=6.5 Hz, 3H).

### Example 14: 2-((1-(3-((1R,5S,6r)-6-cyano-3-azabicyclo[3.1.0]hexan-3-yl)-2,7-dimethyl-1-oxo-1,2-dihydroisoquinolin-5-yl)ethyl)amino)benzoic acid (Enantiomer 1)

### Example 15: 2-((1-(3-((1R,5S,6r)-6-cyano-3-azabicyclo[3.1.0]hexan-3-yl)-2,7-dimethyl-1-oxo-1,2-dihydroisoquinolin-5-yl)ethyl)amino)benzoic acid (Enantiomer 2)

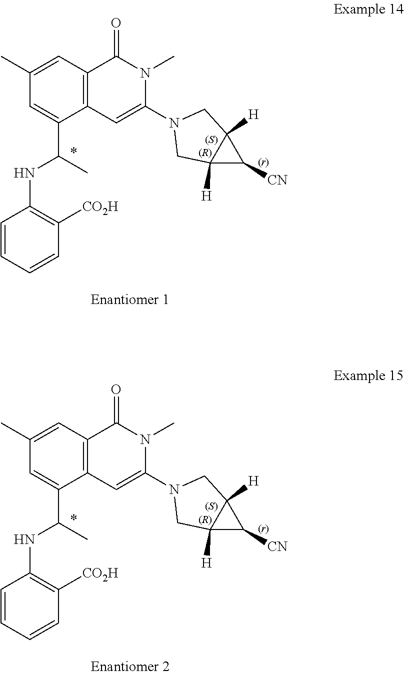

Prepared in a manner similar to Examples 8 and 9 using (1R,5S,6r)-3-azabicyclo[3.1.0]hexane-6-carbonitrile in place of 1-(2,2,2-trifluoroethyl)piperazine dihydrochloride in Step 1:

2-((1-(3-((1R,5S,6r)-6-cyano-3-azabicyclo[3.1.0]hexan-3-yl)-2,7-dimethyl-1-oxo-1,2-dihydroisoquinolin-5-yl)ethyl)amino)benzoic acid Enantiomer 1 (99.9% ee). MS: (ES) m/z)=441.0 [M−H]⁻. ¹H NMR: (400 MHz, Methanol-d4) δ 7.96 (s, 1H), 7.91 (d, J=7.8 Hz, 1H), 7.57 (d, J=2.0 Hz, 1H), 7.10-6.95 (m, 1H), 6.61 (s, 1H), 6.55-6.50 (m, 1H), 6.33 (d, J=8.4 Hz, 1H), 5.18-4.95 (m, 1H), 4.63 (s, 3H), 3.52-3.35 (m, 5H), 3.15-3.05 (m, 2H), 2.35-2.20 (m, 5H), 1.63 (d, J=6.7 Hz, 3H).

2-((1-(3-((1R,5S,6r)-6-cyano-3-azabicyclo[3.1.0]hexan-3-yl)-2,7-dimethyl-1-oxo-1,2-dihydroisoquinolin-5-yl)ethyl)amino)benzoic acid Enantiomer 2 (99.9% ee). MS: (ES) m/z)=441.0 [M−H]⁻. ¹H NMR: (400 MHz, Methanol-d4) δ 7.97 (s, 1H), 7.91 (d, J=7.8 Hz, 1H), 7.55 (d, J=2.0 Hz, 1H), 7.10-6.95 (m, 1H), 6.61 (s, 1H), 6.55-6.50 (m, 1H), 6.33 (d, J=8.4 Hz, 1H), 5.18-4.95 (m, 1H), 4.63 (s, 3H), 3.52-3.35 (m, 5H), 3.15-3.05 (m, 2H), 2.35-2.20 (m, 5H), 1.63 (d, J=6.7 Hz, 3H).

### Example 16: 2-((1-(3-((1R,5S,6r)-6-cyano-3-azabicyclo[3.1.0]hexan-3-yl)-2,7-dimethyl-1-oxo-1,2-dihydroisoquinolin-5-yl)ethyl)amino)benzoic acid (Enantiomer 1)

### Example 17: 2-((1-(3-((1R,5S,6r)-6-cyano-3-azabicyclo[3.1.0]hexan-3-yl)-2,7-dimethyl-1-oxo-1,2-dihydroisoquinolin-5-yl)ethyl)amino)benzoic acid (Enantiomer 2)

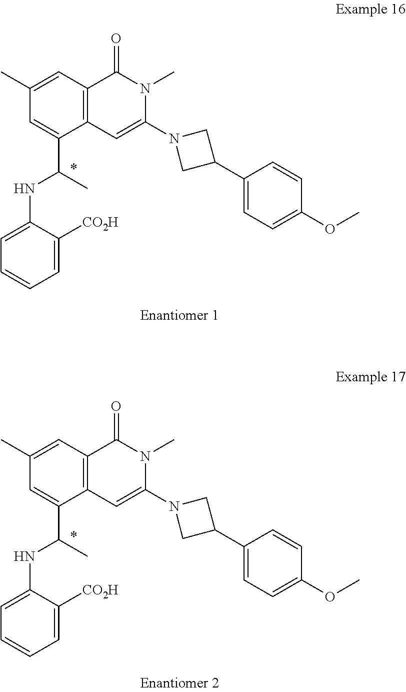

Prepared in a manner similar to Examples 8 and 9 using 3-(4-methoxyphenyl) azetidine in place of 1-(2,2,2-trifluoroethyl)piperazine dihydrochloride in Step 1:

2-((1-(3-((1R,5S,6r)-6-cyano-3-azabicyclo[3.1.0]hexan-3-yl)-2,7-dimethyl-1-oxo-1,2-dihydroisoquinolin-5-yl)ethyl)amino)benzoic acid Enantiomer 1 (99.9% ee). MS: (ES⁺) m/z=498.2 [M+H]⁺. ¹H NMR: (300 MHz, DMSO-d6): δ 8.47 (s, 1H), 7.90-7.70 (m, 2H), 7.39-7.28 (m, 3H), 7.25-7.15 (m, 1H), 7.00-6.90 (m, 2H), 6.55-4.45 (m, 1H), 6.36 (d, J=8.5 Hz, 1H), 5.94 (s, 1H), 5.15-5.05 (m, 1H), 4.40-4.30 (m, 2H), 3.85-3.75 (m, 3H), 3.73 (s, 3H), 3.44 (s, 3H), 2.28 (s, 3H), 1.52 (d, J=6.5 Hz, 3H).

2-((1-(3-((1R,5S,6r)-6-cyano-3-azabicyclo[3.1.0]hexan-3-yl)-2,7-dimethyl-1-oxo-1,2-dihydroisoquinolin-5-yl)ethyl)amino)benzoic acid Enantiomer 1 (99.9% ee). MS: (ES⁺) m/z=498.2 [M+H]⁺. ¹H NMR (300 MHz, DMSO-d6): δ 8.45 (s, 1H), 7.90-7.70 (m, 2H), 7.39-7.28 (m, 3H), 7.25-7.15 (m, 1H), 6.99-6.89 (m, 2H), 6.55-4.45 (m, 1H), 6.37 (d, J=8.5 Hz, 1H), 5.93 (s, 1H), 5.15-5.05 (m, 1H), 4.40-4.30 (m, 2H), 3.85-3.75 (m, 3H), 3.73 (s, 3H), 3.44 (s, 3H), 2.28 (s, 3H), 1.53 (d, J=6.4 Hz, 3H).

### Example 18: 2-((1-(3-(5,7-dihydro-6H-pyrrolo[3,4-b]pyridin-6-yl)-2,7-dimethyl-1-oxo-1,2-dihydroisoquinolin-5-yl)ethyl)amino)benzoic acid (Enantiomer 1)

### Example 19: 2-((1-(3-(5,7-dihydro-6H-pyrrolo[3,4-b]pyridin-6-yl)-2,7-dimethyl-1-oxo-1,2-dihydroisoquinolin-5-yl)ethyl)amino)benzoic acid (Enantiomer 2)

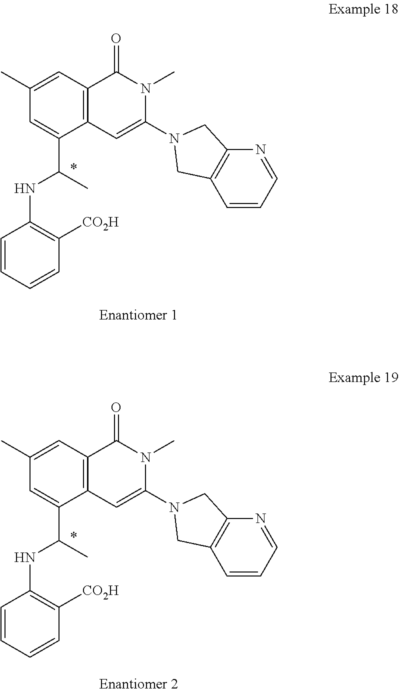 [SMILES: Cc1cc(C(C)Nc2ccccc2C(=O)O)c2cc(N3Cc4cccnc4C3)n(C)c(=O)c2c1 ; Cc1cc(C(C)Nc2ccccc2C(=O)O)c2cc(N3Cc4cccnc4C3)n(C)c(=O)c2c1]

Prepared in a manner similar to Examples 8 and 9 using 6,7-dihydro-5H-pyrrolo[3,4-b]pyridine in place of 1-(2,2,2-trifluoroethyl)piperazine dihydrochloride in Step 1:

2-((1-(3-(5,7-dihydro-6H-pyrrolo[3,4-b]pyridin-6-yl)-2,7-dimethyl-1-oxo-1,2-dihydroisoquinolin-5-yl)ethyl)amino)benzoic acid Enantiomer 1 (99.9% ee). MS: (ES⁺) m/z=455.1 [M+H]⁺. ¹H NMR: (400 MHz, DMSO-d6) δ 13.35-12.00 (m, 1H), 8.56-8.44 (m, 2H), 7.88 (d, J=1.7 Hz, 1H), 7.85-7.80 (m, 2H), 7.41 (d, J=1.9 Hz, 1H), 7.35-7.25 (m, 1H), 7.21-7.09 (m, 1H), 6.74 (s, 1H), 6.52-6.48 (m, 1H), 6.36 (d, J=8.4 Hz, 1H), 5.24-5.20 (m, 1H), 4.76-4.53 (m, 4H), 3.59 (s, 3H), 2.32 (s, 3H), 1.52 (d, J=6.4 Hz, 3H).

2-((1-(3-(5,7-dihydro-6H-pyrrolo[3,4-b]pyridin-6-yl)-2,7-dimethyl-1-oxo-1,2-dihydroisoquinolin-5-yl)ethyl)amino)benzoic acid Enantiomer 2 (99.9% ee). MS: (ES⁺) m/z=455.1 [M+H]⁺. ¹H NMR: (400 MHz, DMSO-d6) δ 13.35-12.00 (m, 1H), 8.56-8.44 (m, 2H), 7.88 (d, J=1.7 Hz, 1H), 7.85-7.80 (m, 2H), 7.41 (d, J=1.9 Hz, 1H), 7.35-7.25 (m, 1H), 7.21-7.09 (m, 1H), 6.74 (s, 1H), 6.52-6.48 (m, 1H), 6.36 (d, J=8.4 Hz, 1H), 5.24-5.20 (m, 1H), 4.76-4.53 (m, 4H), 3.59 (s, 3H), 2.32 (s, 3H), 1.52 (d, J=6.4 Hz, 3H).

### Example 20: 2-((1-(2,7-dimethyl-3-(6-methylpyridin-3-yl)-1-oxo-1,2-dihydroisoquinolin-5-yl)ethyl)amino)benzoic acid (Enantiomer 1)

### Example 21: 2-((1-(2,7-dimethyl-3-(6-methylpyridin-3-yl)-1-oxo-1,2-dihydroisoquinolin-5-yl)ethyl)amino)benzoic acid (Enantiomer 2)

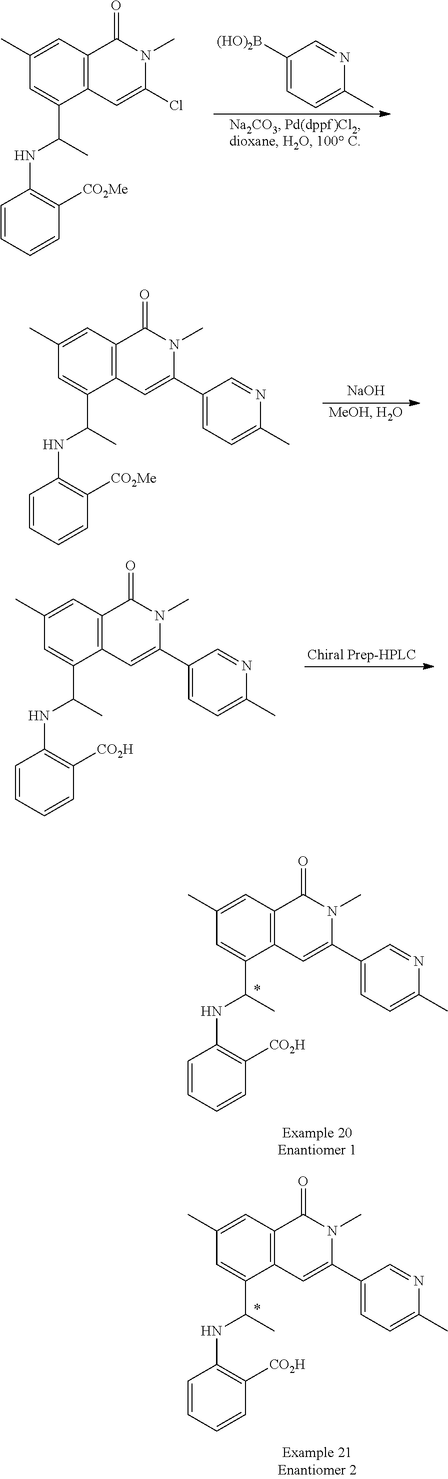

### Step 1: Preparation of methyl 2-((1-(2,7-dimethyl-3-(6-methylpyridin-3-yl)-1-oxo-1,2-dihydroisoquinolin-5-yl)ethyl)amino)benzoate

A solution of methyl 2-((1-(3-chloro-2,7-dimethyl-1-oxo-1,2-dihydroisoquinolin-5-yl)ethyl)amino)benzoate (100 mg, 0.26 mmol) and (6-methylpyridin-3-yl) boronic acid (89 mg, 0.65 mmol) in dioxane (5 mL) and H₂O (1 mL) at room temperature under a nitrogen atmosphere was treated with Na₂CO₃ (83 mg, 0.78 mmol) and Pd(dppf)Cl₂ (29 mg, 0.039 mmol). The resulting mixture was stirred overnight at 100° C. under a nitrogen atmosphere and then concentrated under reduced pressure. The residue was purified by prep TLC (PE/EA=1:1) to afford methyl 2-((1-(2,7-dimethyl-3-(6-methylpyridin-3-yl)-1-oxo-1,2-dihydroisoquinolin-5-yl)ethyl)amino)benzoate (58 mg, 50%) as an off-white solid. MS: (ES⁺) m/z=442.2 [M+H]⁺.

### Step 2: Preparation of 2-((1-(2,7-dimethyl-3-(6-methylpyridin-3-yl)-1-oxo-1,2-dihydroisoquinolin-5-yl)ethyl)amino)benzoic acid

A solution of methyl 2-((1-(2,7-dimethyl-3-(6-methylpyridin-3-yl)-1-oxo-1,2-dihydroisoquinolin-5-yl)ethyl)amino)benzoate (58 mg, 0.13 mmol) in MeOH (10 mL) and H₂O (2 mL) was treated with NaOH (26 mg, 0.66 mmol) at room temperature and the resulting mixture was stirred overnight at 50° C. The mixture was adjusted to pH 7 with aqueous 1 M HCl and extracted with EtOAc (3×25 mL). The combined organic layers were washed with brine (3×15 mL), dried over anhydrous Na₂SO₄, and then concentrated under reduced pressure. The crude residue was purified by prep HPLC (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 10% B to 38% B in 9 min, 38% B; Wavelengths: 254 nm) to afford 2-((1-(2,7-dimethyl-3-(6-methylpyridin-3-yl)-1-oxo-1,2-dihydroisoquinolin-5-yl)ethyl)amino)benzoic acid (11.5 mg, 20%) as a white solid. MS: (ES⁺) m/z=428.1 [M+H]⁺.

### Step 3: Preparation of 2-((1-(2,7-dimethyl-3-(6-methylpyridin-3-yl)-1-oxo-1,2-dihydroisoquinolin-5-yl)ethyl)amino)benzoic acid (Enantiomers 1 and 2)

A racemic mixture of 2-((1-(2,7-dimethyl-3-(6-methylpyridin-3-yl)-1-oxo-1,2-dihydroisoquinolin-5-yl)ethyl)amino)benzoic acid (20 mg, 0.047 mmol) was separated by chiral HPLC (CHIRAL ART Cellulose-SC, 2*25 cm, 5 μm; Mobile Phase A: Hex (0.1% FA)-HPLC, Mobile Phase B: EtOH-HPLC; Flow rate: 20 mL/min; Gradient: 15% B to 15% B in 20 min; Wavelengths: 220/254 nm; RT1 (min): 20.317; RT2 (min): 22.801; Sample Solvent: EtOH-HPLC; Injection Volume: 1 mL) to afford each enantiomer of 2-((1-(2,7-dimethyl-3-(6-methylpyridin-3-yl)-1-oxo-1,2-dihydroisoquinolin-5-yl)ethyl)amino)benzoic acid as a white solid:

2-((1-(2,7-dimethyl-3-(6-methylpyridin-3-yl)-1-oxo-1,2-dihydroisoquinolin-5-yl)ethyl)amino)benzoic acid Enantiomer 1 (2.8 mg, 13%, 99.7% ee). MS: (ES⁺) m/z=428.1 [M+H]⁺. ¹H NMR: (400 MHz, DMSO-d6) δ 8.72 (s, 1H), 8.38 (d, J=5.9 Hz, 1H), 8.06-7.98 (s, 2H), 7.84-7.78 (m, 1H), 7.49 (d, J=2.0 Hz, 2H), 7.17-7.15 (m, 1H), 6.92 (s, 1H), 6.56-6.48 (m, 1H), 6.34 (d, J=8.4 Hz, 1H), 5.30-5.22 (m, 1H), 3.58-3.35 (m, 3H), 2.59 (s, 3H), 2.38 (s, 3H), 1.51 (d, J=6.5 Hz, 3H).

2-((1-(2,7-dimethyl-3-(6-methylpyridin-3-yl)-1-oxo-1,2-dihydroisoquinolin-5-yl)ethyl)amino)benzoic acid Enantiomer 2 (2 mg, 10%, 99.1% ee). MS: (ES⁺) m/z=428.1 [M+H]⁺. ¹H NMR: (400 MHz, DMSO-d6) δ 8.72 (s, 1H), 8.38 (d, J=5.9 Hz, 1H), 8.06-7.98 (m, 2H), 7.84-7.78 (m, 1H), 7.49 (d, J=2.0 Hz, 2H), 7.17-7.15 (m, 1H), 6.93 (s, 1H), 6.56-6.48 (m, 1H), 6.34 (d, J=8.5 Hz, 1H), 5.30-5.22 (m, 1H), 3.58-3.35 (m, 3H), 2.59 (s, 3H), 2.38 (s, 3H), 1.51 (d, J=6.5 Hz, 3H).

### Example 22: 6-chloro-3-((1-(3-(4,4-dimethylcyclohex-1-en-1-yl)-2,7-dimethyl-1-oxo-1,2-dihydroisoquinolin-5-yl)ethyl)amino)picolinic acid (Enantiomer 1)

### Example 23: 6-chloro-3-((1-(3-(4,4-dimethylcyclohex-1-en-1-yl)-2,7-dimethyl-1-oxo-1,2-dihydroisoquinolin-5-yl)ethyl)amino)picolinic acid (Enantiomer 2)

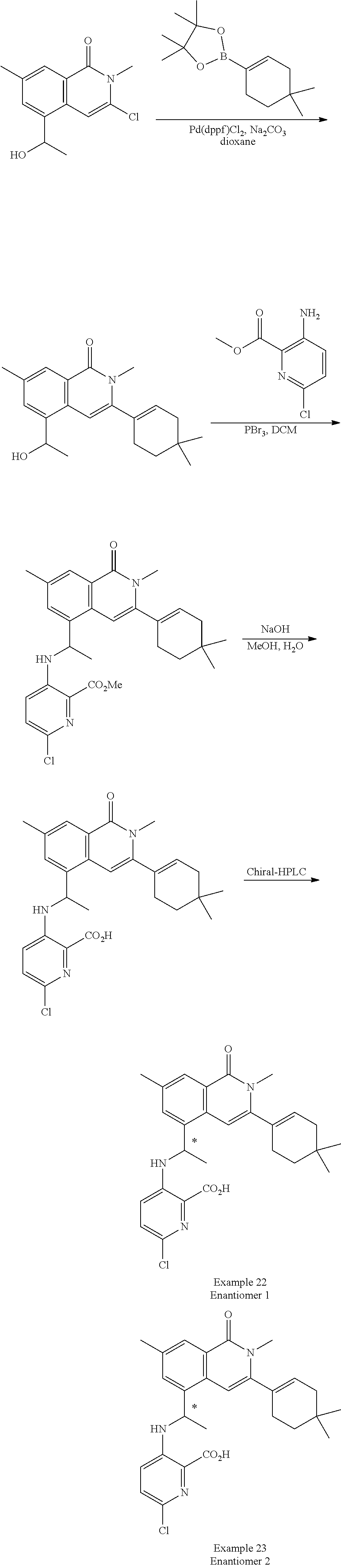

### Step 1: Preparation of 3-(4,4-dimethylcyclohex-1-en-1-yl)-5-(1-hydroxyethyl)-2,7-dimethylisoquinolin-1-one

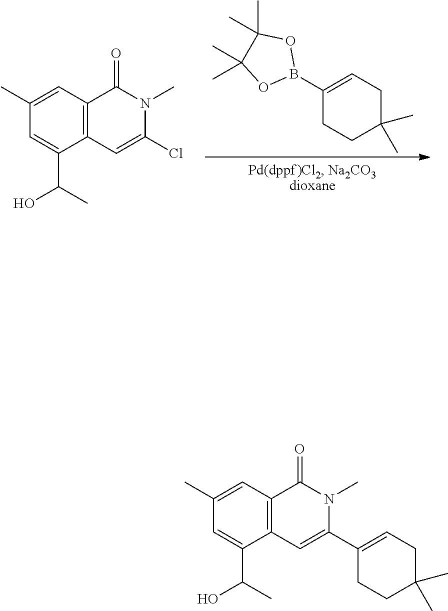

A mixture of 3-chloro-5-(1-hydroxyethyl)-2,7-dimethylisoquinolin-1-one (200 mg, 0.80 mmol), 2-(4,4-dimethylcyclohex-1-en-1-yl)-4,4,5,5-tetramethyl-1,3,2-dioxaborolane (281 mg, 1.19 mmol), sodium methaneperoxoate sodium (170 mg, 1.59 mmol) and Pd(dppf)Cl₂ (324 mg, 0.40 mmol) in dioxane (2 mL) and water (1 mL) was stirred for 2 h at 100° C. under a nitrogen atmosphere. The resulting mixture was extracted with EtOAc (2×20 mL). The combined organic layers were washed with brine (2×30 mL), dried over anhydrous Na₂SO₄, and then concentrated under reduced pressure. The residue was purified by prep TLC (PE/EA=1:1) to afford 3-(4,4-dimethylcyclohex-1-en-1-yl)-5-(1-hydroxyethyl)-2,7-dimethylisoquinolin-1-one (160 mg, 61%) as a yellow oil. MS: (ES⁺) m/z=326.5 [M+H]⁺.

### Step 2: Preparation of methyl 6-chloro-3-((1-(3-(4,4-dimethylcyclohex-1-en-1-yl)-2,7-dimethyl-1-oxo-1,2-dihydroisoquinolin-5-yl)ethyl)amino)picolinate

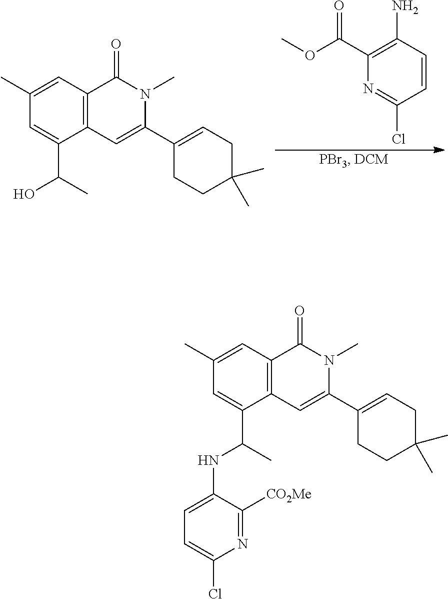

A mixture of 3-(4,4-dimethylcyclohex-1-en-1-yl)-5-(1-hydroxyethyl)-2,7-dimethylisoquinolin-1-one (120 mg, 0.37 mmol) DCM (2 mL) was treated with PBr₃ (200 mg, 0.74 mmol) at 0° C. The resulting mixture was stirred for 2 h at room temperature. The resulting mixture was quenched with H₂O (20 mL). The resulting mixture was extracted with EtOAc (2×20 mL). The combined organic layers were washed with brine (2×20 mL), dried over anhydrous Na₂SO₄, and then concentrated under reduced pressure. The crude intermediate was placed in ACN (3 mL) and methyl 3-amino-6-chloropyridine-2-carboxylate (172 mg, 0.92 mmol) was added in portions at room temperature. The resulting mixture was stirred overnight at 80° C. The resulting mixture was quenched with H₂O (20 mL). The resulting mixture was extracted with EtOAc (2×20 mL). The combined organic layers were washed with brine (2×20 mL), dried over anhydrous Na₂SO₄, and then concentrated under reduced pressure. The residue was purified by prep TLC (PE/EA=1:1) to afford methyl 6-chloro-3-((1-(3-(4,4-dimethylcyclohex-1-en-1-yl)-2,7-dimethyl-1-oxo-1,2-dihydroisoquinolin-5-yl)ethyl)amino)picolinate (120 mg, 65%) as a yellow oil. MS: (ES⁺) m/z=494.0 [M+H]⁺.

### Step 3: Preparation of 6-chloro-3-((1-(3-(4,4-dimethylcyclohex-1-en-1-yl)-2,7-dimethyl-1-oxo-1,2-dihydroisoquinolin-5-yl)ethyl)amino)picolinic acid

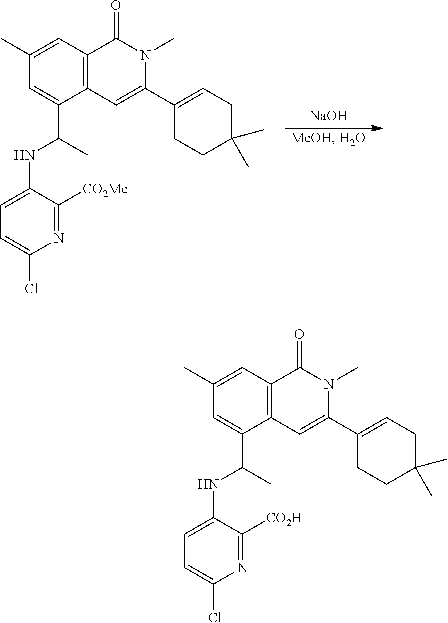

A mixture of methyl 6-chloro-3-((1-(3-(4,4-dimethylcyclohex-1-en-1-yl)-2,7-dimethyl-1-oxo-1,2-dihydroisoquinolin-5-yl)ethyl)amino)picolinate (80 mg, 0.16 mmol), MeOH (2.5 mL), H₂O (0.5 mL) and NaOH (13 mg, 0.32 mmol) was stirred overnight at 50° C. The mixture was adjusted to pH 5 by the addition of an aqueous NaH₂PO₄ solution. The resulting mixture was extracted with EtOAc (2×20 mL). The combined organic layers were washed with brine (2×20 mL), dried over anhydrous Na₂SO₄, and then concentrated under reduced pressure. The residue was purified by reverse phase flash chromatography (Column, C18 silica gel; mobile phase, MeCN in Water (0.1% FA), 5% to 100% gradient in 40 min; detector, UV 254 nm) to afford 6-chloro-3-((1-(3-(4,4-dimethylcyclohex-1-en-1-yl)-2,7-dimethyl-1-oxo-1,2-dihydroisoquinolin-5-yl)ethyl)amino)picolinic acid (40 mg, 51%) as a colorless oil. MS: (ES⁺) m/z=480.2 [M+H]⁺

### Step 4: Preparation of 6-chloro-3-((1-(3-(4,4-dimethylcyclohex-1-en-1-yl)-2,7-dimethyl-1-oxo-1,2-dihydroisoquinolin-5-yl)ethyl)amino)picolinic acid (Enantiomers 1 and 2)

A racemic mixture of 6-chloro-3-((1-(3-(4,4-dimethylcyclohex-1-en-1-yl)-2,7-dimethyl-1-oxo-1,2-dihydroisoquinolin-5-yl)ethyl)amino)picolinic acid (100 mg, 0.21 mmol) was purified by prep chiral HPLC (Column: CHIRALPAK IC-3, 4.6*50 mm, 3 μm; Mobile Phase: Hex (0.1% TFA): EtOH=60:40; Flow rate: 1 mL/min) to afford each enantiomer of 6-chloro-3-((1-(3-(4,4-dimethylcyclohex-1-en-1-yl)-2,7-dimethyl-1-oxo-1,2-dihydroisoquinolin-5-yl)ethyl)amino)picolinic acid as an off-white solid:

6-chloro-3-((1-(3-(4,4-dimethylcyclohex-1-en-1-yl)-2,7-dimethyl-1-oxo-1,2-dihydroisoquinolin-5-yl)ethyl)amino)picolinic acid Enantiomer 1 (15.3 mg, 15%, >93.1% ee). MS: (ES⁺) m/z=480.2 [M+H]⁺. ¹H NMR: (300 MHz, DMSO-d6) δ 13.08 (s, 1H), 8.41 (s, 1H), 7.94 (s, 1H), 7.43 (s, 1H), 7.32 (d, J=8.9 Hz, 1H), 6.89 (d, J=9.0 Hz, 1H), 6.61 (s, 1H), 5.85 (s, 1H), 5.25-5.15 (m, 1H), 3.41 (s, 3H), 2.35 (s, 3H), 2.31-2.23 (m, 2H), 2.10-1.99 (m, 2H), 1.61-1.51 (m, 5H), 1.10-0.99 (m, 6H).

6-chloro-3-((1-(3-(4,4-dimethylcyclohex-1-en-1-yl)-2,7-dimethyl-1-oxo-1,2-dihydroisoquinolin-5-yl)ethyl)amino)picolinic acid Enantiomer 2 (5.8 mg, 5%, >97.5% ee). MS: (ES⁺) m/z=480.2 [M+H]⁺. ¹H NMR: (300 MHz, DMSO-d6) δ 13.08 (s, 1H), 8.55 (s, 1H), 7.93 (s, 1H), 7.43 (s, 1H), 7.32 (d, J=8.9 Hz, 1H), 6.86 (d, J=8.9 Hz, 1H), 6.61 (s, 1H), 5.86 (s, 1H), 5.25-5.15 (s, 1H), 3.41 (s, 3H), 2.35 (s, 3H), 2.31-2.23 (m, 2H), 2.10-1.99 (m, 2H), 1.57-1.45 (m, 5H), 1.10-0.99 (m, 6H).

### Intermediate 2

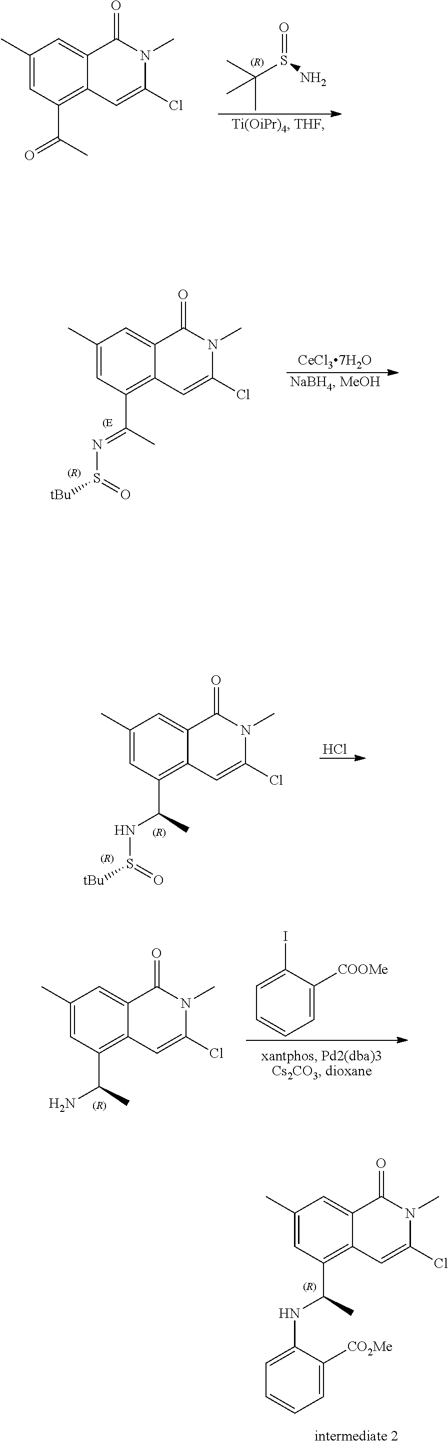

### Step 1: Preparation of methyl (R)-2-((1-(3-chloro-2,7-dimethyl-1-oxo-1,2-dihydroisoquinolin-5-yl)ethyl)amino)benzoate

To a stirred solution of 5-acetyl-3-chloro-2,7-dimethylisoquinolin-1-one (9.8 g, 39.2 mmol) and (R)-2-methylpropane-2-sulfinamide (23.78 g, 196.2 mmol) in THF (200 mL) was added Ti(Oi-Pr)₄ (55.78 g, 196.2 mmol). The resulting mixture was stirred overnight at 80° C. under a nitrogen atmosphere. The reaction was quenched with saturated aqueous sodium chloride (200 mL). The resulting mixture was filtered and the filter cake was washed with ethyl acetate (3×300 mL). The filtrate was extracted with ethyl acetate (3×300 mL). The combined organic layers were washed with H₂O (3×200 mL), dried over anhydrous sodium sulfate, and then concentrated under reduced pressure. The residue was purified by silica gel column chromatography (PE/EA=3:2) to afford (R)—N-[(1E)-1-(3-chloro-2,7-dimethyl-1-oxoisoquinolin-5-yl)ethylidene]-2-methylpropane-2-sulfinamide (9 g, 64%) as a yellow solid. MS: (ES⁺) m/z=353.1 [M+H]⁺.

### Step 2: Preparation of (R)—N-[1-(3-chloro-2,7-dimethyl-1-oxoisoquinolin-5-yl)ethyl]-2-methylpropane-2-sulfinamide

To a stirred solution of (R)—N-[(1E)-1-(3-chloro-2,7-dimethyl-1-oxoisoquinolin-5-yl)ethylidene]-2-methylpropane-2-sulfinamide (10.5 g, 29.76 mmol) and CeCl₃·7H₂O (16.63 g, 44.63 mmol) in MeOH (120 mL) was added NaBH₄ (2.81 g, 74.39 mmol) at −78° C. The resulting solution was stirred for 2 h at room temperature. The reaction mixture was quenched with saturated aqueous ammonium chloride (150 mL) and extracted with ethyl acetate (3×200 mL). The combined organic layers were washed with brine (2×200 mL), dried over anhydrous sodium sulfate, and then concentrated under reduced pressure. The residue was purified by silica gel column chromatography (PE/EA=1:2) and then purified further by HP-Flash (25%-55% ACN in H₂O (0.1% FA) in 45 min) to afford (R)—N-[1-(3-chloro-2,7-dimethyl-1-oxoisoquinolin-5-yl)ethyl]-2-methylpropane-2-sulfinamide (6.5 g, 61%) as an off-white solid. MS: (ES⁺) m/z=355.0 [M+H]⁺.

### Step 3: (R)-5-(1-aminoethyl)-3-chloro-2,7-dimethylisoquinolin-1-one hydrochloride

A mixture of (R)—N-[(1R)-1-(3-chloro-2,7-dimethyl-1-oxoisoquinolin-5-yl)ethyl]-2-methylpropane-2-sulfinamide (240 mg, 0.68 mmol) in 1:1 4 M HCl in 1,4-dioxane/CH₂Cl₂ (10 mL) was stirred overnight at room temperature under a nitrogen atmosphere. The resulting mixture was concentrated under reduced pressure to afford 5-[(1R)-1-aminoethyl]-3-chloro-2,7-dimethylisoquinolin-1-one hydrochloride (165 mg, 85%) as a white solid. The crude product was used in the next step directly without further purification. MS: (ES⁺) m/z=250.0 [M+H]⁺.

### Step 4: Preparation of methyl (R)-2-((1-(3-chloro-2,7-dimethyl-1-oxo-1,2-dihydroisoquinolin-5-yl)ethyl)amino)benzoate

A mixture of 5-[(1R)-1-aminoethyl]-3-chloro-2,7-dimethylisoquinolin-1-one hydrochloride (165 mg, 0.66 mmol), methyl 2-iodobenzoate (344 mg, 1.20 mmol), Cs₂CO₃ (1.27 g, 3.6 mmol), Xantphos (138 mg, 0.24 mmol) and Pd₂(dba)₃ (109.8 mg, 0.12 mmol) in 1,4-dioxane (15 mL) was stirred at 90° C. for 3 h under a nitrogen atmosphere. The reaction mixture was cooled to room temperature, concentrated under reduced pressure and purified by prep TLC (PE/EA=4:1) to afford methyl (R)-2-((1-(3-chloro-2,7-dimethyl-1-oxo-1,2-dihydroisoquinolin-5-yl)ethyl)amino)benzoate (170 mg, 67%) as a yellow solid. MS: (ES⁺) m/z=385.0 [M+H]⁺.

### Example 24: (R)-6-chloro-3-((1-(3-(4-(4-fluorophenyl)piperazin-1-yl)-2,7-dimethyl-1-oxo-1,2-dihydroisoquinolin-5-yl)ethyl)amino)picolinic acid

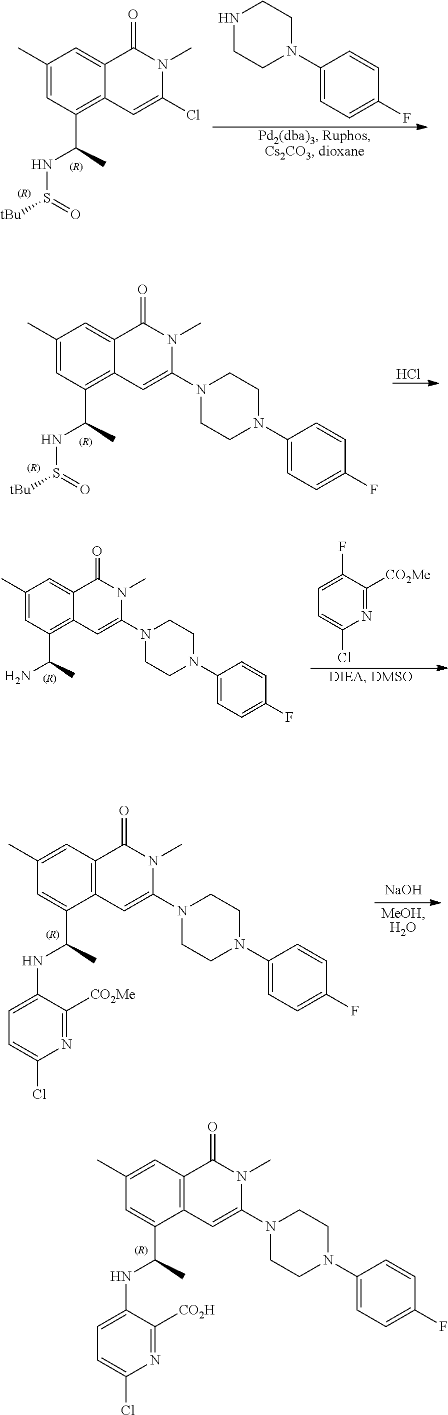

### Step 1: Preparation of (R)—N—((R)-1-(3-(4-(4-fluorophenyl)piperazin-1-yl)-2,7-dimethyl-1-oxo-1,2-dihydroisoquinolin-5-yl)ethyl)-2-methylpropane-2-sulfinamide

A solution of (R)—N-[(1R)-1-(3-chloro-2,7-dimethyl-1-oxoisoquinolin-5-yl)ethyl]-2-methylpropane-2-sulfinamide (280 mg, 0.79 mmol) in dioxane (10 mL) was treated with Cs₂CO₃ (771 mg, 2.37 mmol), Ruphos (73 mg, 0.16 mmol), para-fluorophenylpiperazine (284 mg, 1.58 mmol) and Pd₂(dba)₃ (72 mg, 0.079 mmol). The resulting mixture was stirred overnight at 100° C. under a nitrogen atmosphere and then concentrated under reduced pressure. The residue was purified by prep TLC (CH₂Cl₂/MeOH=12:1) to afford (R)—N—((R)-1-(3-(4-(4-fluorophenyl)piperazin-1-yl)-2,7-dimethyl-1-oxo-1,2-dihydroisoquinolin-5-yl)ethyl)-2-methylpropane-2-sulfinamide (200 mg, 50%) as a yellow solid. MS: (ES⁺) m/z=499.2 [M+H]⁺.

### Step 2: Preparation of (R)-5-(1-aminoethyl)-3-(4-(4-fluorophenyl)piperazin-1-yl)-2,7-dimethylisoquinolin-1 (2H)-one

A mixture of (R)—N—((R)-1-(3-(4-(4-fluorophenyl)piperazin-1-yl)-2,7-dimethyl-1-oxo-1,2-dihydroisoquinolin-5-yl)ethyl)-2-methylpropane-2-sulfinamide (200 mg, 0.40 mmol) in 4 M HCl in dioxane (15 mL) was stirred for 2 h at room temperature. The resulting mixture was concentrated under reduce pressure. The residue was purified by prep TLC (CH₂Cl₂/MeOH=10:1) to afford (R)-5-(1-aminoethyl)-3-(4-(4-fluorophenyl)piperazin-1-yl)-2,7-dimethylisoquinolin-1 (2H)-one (150 mg, 94%) as a yellow solid. MS: (ES⁺) m/z=395.2 [M+H]⁺.

### Step 3: Preparation of methyl (R)-6-chloro-3-((1-(3-(4-(4-fluorophenyl)piperazin-1-yl)-2,7-dimethyl-1-oxo-1,2-dihydroisoquinolin-5-yl)ethyl)amino)picolinate

To a stirred solution of (R)-5-(1-aminoethyl)-3-(4-(4-fluorophenyl)piperazin-1-yl)-2,7-dimethylisoquinolin-1 (2H)-one (150 mg, 0.38 mmol) and methyl 6-chloro-3-fluoropyridine-2-carboxylate (108 mg, 0.57 mmol) in DMSO (10 mL) was added DIEA (246 mg, 1.90 mmol) dropwise at room temperature. The resulting mixture was stirred overnight at 100° C. The reaction was quenched with H₂O (20 mL) and extracted with EtOAc (3×30 mL). The combined organic layers were washed with brine (3×15 mL), dried over anhydrous Na₂SO₄, and then concentrated under reduced pressure. The residue was purified by prep TLC (CH₂Cl₂/MeOH=15:1) to afford methyl (R)-6-chloro-3-((1-(3-(4-(4-fluorophenyl)piperazin-1-yl)-2,7-dimethyl-1-oxo-1,2-dihydroisoquinolin-5-yl)ethyl)amino)picolinate (130 mg, 60%) as a yellow solid. MS: (ES⁺) m/z=564.2 [M+H]⁺.

### Step 4: Preparation of (R)-6-chloro-3-((1-(3-(4-(4-fluorophenyl)piperazin-1-yl)-2,7-dimethyl-1-oxo-1,2-dihydroisoquinolin-5-yl)ethyl)amino)picolinic acid

A solution of methyl (R)-6-chloro-3-((1-(3-(4-(4-fluorophenyl)piperazin-1-yl)-2,7-dimethyl-1-oxo-1,2-dihydroisoquinolin-5-yl)ethyl)amino)picolinate (120 mg, 0.21 mmol) in MeOH (5 mL) and H₂O (1 mL) was treated with NaOH (43 mg, 1.07 mmol). The resulting mixture was stirred overnight at 50° C. The mixture was then acidified to pH 5 with aqueous 1 N HCl. The resulting mixture was diluted with H₂O (15 mL) and extracted with EtOAc (3×30 mL). The combined organic layers were washed with brine (2×10 mL), dried over anhydrous Na₂SO₄, and then concentrated under reduced pressure. The residue was purified by prep TLC (CH₂Cl₂/MeOH=5:1). The crude product was purified by prep HPLC (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 21% B to 51% B in 9 min, 51% B; Wavelengths: 254 nm; RT1 (min): 7.00) to afford (R)-6-chloro-3-((1-(3-(4-(4-fluorophenyl)piperazin-1-yl)-2,7-dimethyl-1-oxo-1,2-dihydroisoquinolin-5-yl)ethyl)amino)picolinic acid (34 mg, 28%) as a white solid. MS: (ES⁺) m/z=550.1 [M+H]⁺. ¹H NMR: (400 MHz, Methanol-d4) δ 8.02 (s, 1H), 7.54 (d, J=1.9 Hz, 1H), 7.17 (d, J=8.9 Hz, 1H), 7.11-6.98 (m, 4H), 6.89 (d, J=8.9 Hz, 1H), 6.56 (s, 1H), 5.17-5.02 (m, 1H), 3.69 (s, 3H), 3.68-3.55 (m, 2H), 3.15-2.80 (m, 6H), 2.40 (s, 3H), 1.69 (d, J=6.6 Hz, 3H).

### Example 25: (R)-6-chloro-3-((1-(3-(4-(4-fluorobenzyl)piperazin-1-yl)-2,7-dimethyl-1-oxo-1,2-dihydroisoquinolin-5-yl)ethyl)amino)picolinic acid

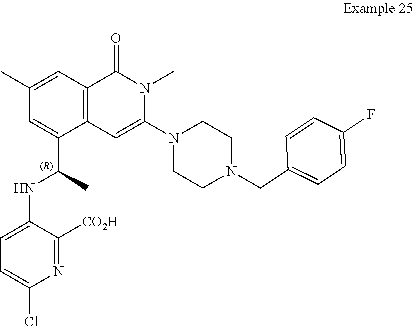

Prepared in a manner similar to Example 24 using 1-(4-fluorobenzyl)piperazine in place of para-fluorophenylpiperazine in Step 1. MS: (ES⁺) m/z=564.1 [M+H]⁺. ¹H NMR: (300 MHz, Methanol-d4): δ 8.01-7.94 (m, 1H), 7.59-7.49 (m, 3H), 7.19-6.92 (m, 3H), 6.71 (d, J=8.8 Hz, 1H), 6.51 (s, 1H), 5.05-4.95 (m, 1H), 4.06 (s, 2H), 3.61 (s, 3H), 3.25-2.90 (m, 8H), 2.37 (s, 3H), 1.63 (d, J=6.6 Hz, 3H).

### Example 26: (R)-6-chloro-3-((1-(3-(6-(4-fluorophenyl)pyridin-3-yl)-2,7-dimethyl-1-oxo-1,2-dihydroisoquinolin-5-yl)ethyl)amino)picolinic acid

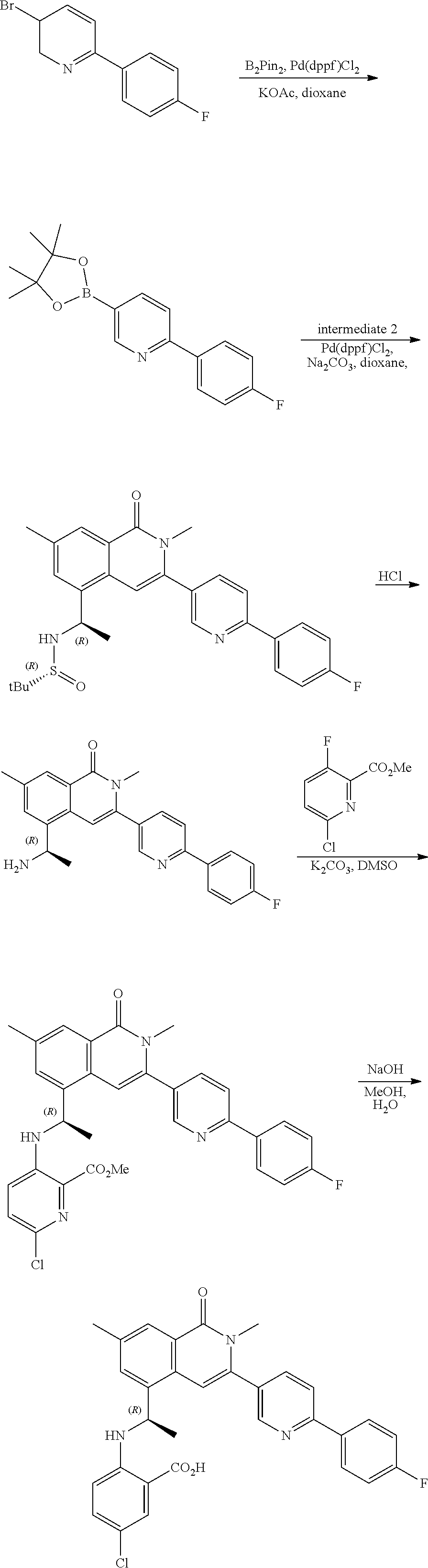

### Step 1: Preparation of 2-(4-fluorophenyl)-5-(4,4,5,5-tetramethyl-1,3,2-dioxaborolan-2-yl)pyridine

To a stirred solution of 5-bromo-2-(4-fluorophenyl)pyridine (300 mg, 1.19 mmol) and 4,4,5,5-tetramethyl-2-(4,4,5,5-tetramethyl-1,3,2-dioxaborolan-2-yl)-1,3,2-dioxaborolane (604 mg, 2.38 mmol) in 1,4-dioxane were added Pd(dppf)Cl₂ (87 mg, 0.12 mmol) and KOAc (233 mg, 2.38 mmol) at room temperature under a nitrogen atmosphere. The resulting mixture was stirred for 1 h at 90° C. and then concentrated under reduced pressure. The residue was purified by prep TLC (PE/EA=1:2) to afford 2-(4-fluorophenyl)-5-(4,4,5,5-tetramethyl-1,3,2-dioxaborolan-2-yl)pyridine (290 mg, 81%) as a white solid. MS: (ES⁺) m/z=300.2 [M+H]⁺.

### Step 2: Preparation of (R)—N—((R)-1-(3-(6-(4-fluorophenyl)pyridin-3-yl)-2,7-dimethyl-1-oxo-1,2-dihydroisoquinolin-5-yl)ethyl)-2-methylpropane-2-sulfinamide

To a stirred solution of (R)—N-[(1R)-1-(3-chloro-2,7-dimethyl-1-oxoisoquinolin-5-yl)ethyl]-2-methylpropane-2-sulfinamide (290 mg, 0.82 mmol) and 2-(4-fluorophenyl)-5-(4,4,5,5-tetramethyl-1,3,2-dioxaborolan-2-yl)pyridine (293 mg, 0.98 mmol) in 1,4-dioxane (16 mL) and H₂O (2 mL) were added Pd(dppf)Cl₂ (59 mg, 0.082 mmol) and Na₂CO₃ (173 mg, 1.63 mmol) at room temperature under a nitrogen atmosphere. The resulting mixture was stirred for 2 h at 100° C. and then concentrated under reduced pressure. The residue was purified by prep TLC (EA) to afford (R)—N—((R)-1-(3-(6-(4-fluorophenyl)pyridin-3-yl)-2,7-dimethyl-1-oxo-1,2-dihydroisoquinolin-5-yl)ethyl)-2-methylpropane-2-sulfinamide (320 mg, 79%) as a yellow solid. MS: (ES⁺) m/z=492.2 [M+H]⁺.

### Step 3: Preparation of (R)-5-(1-aminoethyl)-3-(6-(4-fluorophenyl)pyridin-3-yl)-2,7-dimethylisoquinolin-1 (2H)-one hydrochloride

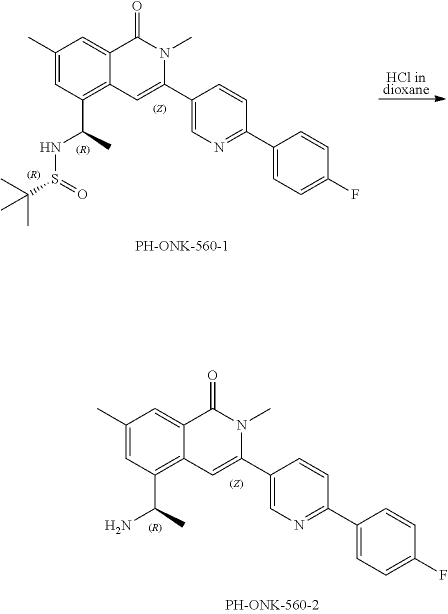

A solution of (R)—N—((R)-1-(3-(6-(4-fluorophenyl)pyridin-3-yl)-2,7-dimethyl-1-oxo-1,2-dihydroisoquinolin-5-yl)ethyl)-2-methylpropane-2-sulfinamide (300 mg, 0.61 mmol) in 4 N HCl in 1,4-dioxane (5 mL) was stirred for 2 h at room temperature under a nitrogen atmosphere. The resulting mixture was concentrated under reduced pressure to afford (R)-5-(1-aminoethyl)-3-(6-(4-fluorophenyl)pyridin-3-yl)-2,7-dimethylisoquinolin-1 (2H)-one hydrochloride (200 mg, 77%). The crude product was used in the next step directly without further purification. MS: (ES⁺) m/z=388.2 [M+H]⁺.

### Step 4: Preparation of methyl (R)-6-chloro-3-((1-(3-(6-(4-fluorophenyl)pyridin-3-yl)-2,7-dimethyl-1-oxo-1,2-dihydroisoquinolin-5-yl)ethyl)amino)picolinate

To a stirred solution of (R)-5-(1-aminoethyl)-3-(6-(4-fluorophenyl)pyridin-3-yl)-2,7-dimethylisoquinolin-1 (2H)-one hydrochloride (200 mg, 0.47 mmol) and methyl 6-chloro-3-fluoropyridine-2-carboxylate (391 mg, 2.06 mmol) in DMSO (5 mL) was added K₂CO₃ (195 mg, 1.41 mmol) at room temperature under a nitrogen atmosphere. The resulting mixture was stirred for 3 h at 120° C. The reaction was quenched with H₂O (10 mL). The aqueous layer was extracted with CH₂Cl₂ (3×10 mL). The combined organic layers were washed with brine (2×10 mL), dried over anhydrous Na₂SO₄ and concentrated under reduced pressure. The residue was purified by prep TLC (PE/EA=1:1) to afford methyl (R)-6-chloro-3-((1-(3-(6-(4-fluorophenyl)pyridin-3-yl)-2,7-dimethyl-1-oxo-1,2-dihydroisoquinolin-5-yl)ethyl)amino)picolinate (140 mg, 48%) as a yellow solid. MS: (ES⁺) m/z=557.2 [M+H]⁺.

### Step 5: Preparation of (R)-6-chloro-3-((1-(3-(6-(4-fluorophenyl)pyridin-3-yl)-2,7-dimethyl-1-oxo-1,2-dihydroisoquinolin-5-yl)ethyl)amino)picolinic acid

To a stirred solution of methyl (R)-6-chloro-3-((1-(3-(6-(4-fluorophenyl)pyridin-3-yl)-2,7-dimethyl-1-oxo-1,2-dihydroisoquinolin-5-yl)ethyl)amino)picolinate (130 mg, 0.23 mmol) and NaOH (46 mg, 1.16 mmol) in MeOH (9 mL) was added H₂O (1.6 mL) at room temperature under a nitrogen atmosphere. The resulting mixture was stirred overnight at 50° C. The mixture was adjusted to pH 6 with aqueous 1 N HCl and concentrated under reduced pressure. The crude product was purified by prep HPLC (Column: XSelect CSH Prep C18 OBD Column, 19*250 mm, 5 μm; Mobile Phase A: Water (0.1% FA), Mobile Phase B: ACN; Flow rate: 20 mL/min; Gradient: 13% B to 40% B in 8 min, 40% B; Wavelengths: 254 nm; RT1 (min): 8.00) to afford (R)-6-chloro-3-((1-(3-(6-(4-fluorophenyl)pyridin-3-yl)-2,7-dimethyl-1-oxo-1,2-dihydroisoquinolin-5-yl)ethyl)amino)picolinic acid (28 mg, 21%) as a white solid. MS: (ES⁺) m/z=543.0 [M+H]⁺. ¹H NMR: (300 MHz, DMSO-d6): δ 8.94-8.88 (m, 1H), 8.44 (d, J=6.1 Hz, 1H), 8.32-8.20 (m, 2H), 8.25-8.10 (m, 2H), 8.04 (d, J=1.8 Hz, 1H), 7.49 (d, J=1.8 Hz, 1H), 7.45-7.28 (m, 3H), 7.00 (s, 1H), 6.92 (d, J=9.0 Hz, 1H), 5.40-5.30 (m, 1H), 3.42 (s, 3H), 2.40 (s, 3H), 1.54 (d, J=6.5 Hz, 3H).

### Example 27: (R)-5-chloro-2-((1-(3-(6-(4-fluorophenyl)pyridin-3-yl)-2,7-dimethyl-1-oxo-1,2-dihydroisoquinolin-5-yl)ethyl)amino)benzoic acid

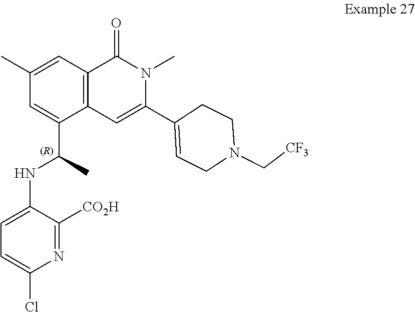

Prepared in a manner similar to Example 26 using 4-(4,4,5,5-tetramethyl-1,3,2-dioxaborolan-2-yl)-1-(2,2,2-trifluoroethyl)-1,2,3,6-tetrahydropyridine in place of 2-(4-fluorophenyl)-5-(4,4,5,5-tetramethyl-1,3,2-dioxaborolan-2-yl)pyridine in Step 2. MS: (ES⁺) m/z=535.0 [M+H]⁺. ¹H NMR: (300 MHz, CD₃OD): δ 8.02 (s, 1H), 7.54 (s, 1H), 7.08 (d, J=8.7 Hz, 1H), 6.84 (s, 1H), 6.77 (d, J=9.0 Hz, 1H), 5.98 (s, 1H), 5.18-5.11 (m, 1H), 3.54 (s, 3H), 3.43 (s, 3H), 3.31-3.20 (m, 2H), 2.98-2.96 (m, 2H), 2.48-2.37 (m, 2H), 2.37 (s, 3H), 1.63 (d, J=6.6 Hz, 3H).

### Example 28: 2-((1-(3-(4,4-dimethylpiperidin-1-yl)-2-ethyl-7-methyl-1-oxo-1,2-dihydroisoquinolin-5-yl)ethyl)amino)benzoic acid (Enantiomer 1)

### Example 29: 2-((1-(3-(4,4-dimethylpiperidin-1-yl)-2-ethyl-7-methyl-1-oxo-1,2-dihydroisoquinolin-5-yl)ethyl)amino)benzoic acid (Enantiomer 2)

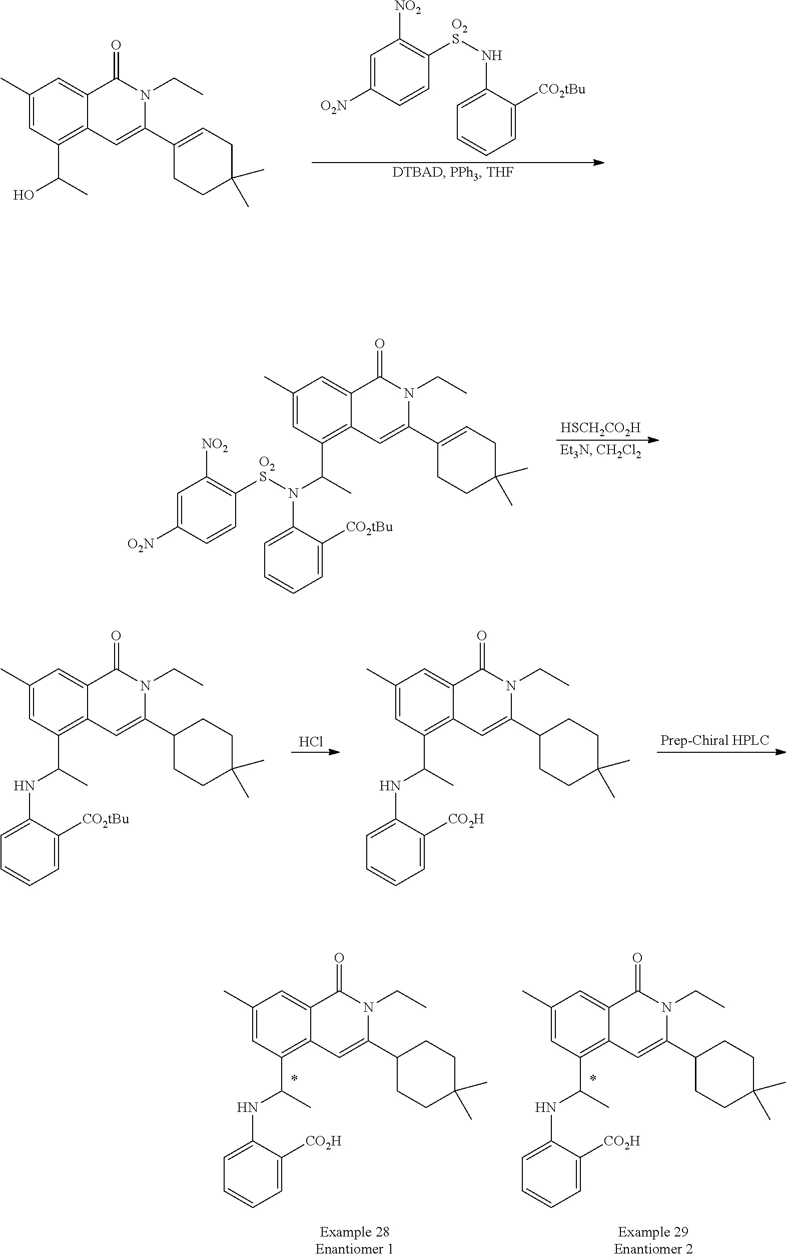

### Step 1: Preparation of tert-butyl 2-((N-(1-(3-(4,4-dimethylpiperidin-1-yl)-2-ethyl-7-methyl-1-oxo-1,2-dihydroisoquinolin-5-yl)ethyl)-2,4-dinitrophenyl)sulfonamido)benzoate

To a solution of 3-(4,4-dimethylpiperidin-1-yl)-2-ethyl-5-(1-hydroxyethyl)-7-methylisoquinolin-1-one (prepared in a manner similar to Example 1, using ethyl iodide in place of iodomethane in Step 3, 400 mg, 1.17 mmol), tert-butyl 2-(2,4-dinitrobenzenesulfonamido)benzoate (494 mg, 1.17 mmol) and triphenylphosphine (612 mg, 2.34 mmol) in tetrahydrofuran (12 mL) was added a solution of DTAD (672 mg, 2.92 mmol) in tetrahydrofuran (3 mL) at 0° C. under a nitrogen atmosphere. The resulting solution was stirred overnight at room temperature and then concentrated under reduced pressure. The residue was purified by prep TLC (PE/EA=3:1) to afford tert-butyl 2-((N-(1-(3-(4,4-dimethylpiperidin-1-yl)-2-ethyl-7-methyl-1-oxo-1,2-dihydroisoquinolin-5-yl)ethyl)-2,4-dinitrophenyl)sulfonamido)benzoate (864 mg, 98%) as a yellow solid. MS: (ES⁺) m/z=748.3 [M+H]⁺.

### Step 2: Preparation of tert-butyl 2-((1-(3-(4,4-dimethylpiperidin-1-yl)-2-ethyl-7-methyl-1-oxo-1,2-dihydroisoquinolin-5-yl)ethyl)amino)benzoate

A mixture of tert-butyl 2-((N-(1-(3-(4,4-dimethylpiperidin-1-yl)-2-ethyl-7-methyl-1-oxo-1,2-dihydroisoquinolin-5-yl)ethyl)-2,4-dinitrophenyl)sulfonamido)benzoate (850 mg, 1.14 mmol), thioglycolic acid (0.16 mL, 2.27 mmol), and triethylamine (0.47 mL, 3.41 mmol) in methylene chloride (20 mL) was stirred at room temperature for 3 h under a nitrogen atmosphere. The mixture was adjusted to pH 7-8 with a saturated aqueous NaHCO₃ solution. The reaction mixture was extracted with methylene chloride (3×100 mL). The combined organic layers were washed with brine (2×100 mL), dried over anhydrous sodium sulfate, and then concentrated under reduced pressure. The residue was purified by prep TLC (PE/EA=4:1) to afford tert-butyl 2-((1-(3-(4,4-dimethylpiperidin-1-yl)-2-ethyl-7-methyl-1-oxo-1,2-dihydroisoquinolin-5-yl)ethyl)amino)benzoate (466 mg, 79%) as a yellow solid. MS: (ES⁺) m/z=518.3 [M+H]⁺.

### Step 3: Preparation of 2-((1-(3-(4,4-dimethylpiperidin-1-yl)-2-ethyl-7-methyl-1-oxo-1,2-dihydroisoquinolin-5-yl)ethyl)amino)benzoic acid

A mixture of tert-butyl 2-((1-(3-(4,4-dimethylpiperidin-1-yl)-2-ethyl-7-methyl-1-oxo-1,2-dihydroisoquinolin-5-yl)ethyl)amino)benzoate (466 mg, 0.90 mmol) in 4 M HCl in 1,4-dioxane (20 mL) was stirred overnight at room temperature under a nitrogen atmosphere. The resulting mixture was concentrated under reduced pressure. The residue was diluted with 5 mL DCM, neutralized with NH₃ in MeOH (7M), and purified by prep TLC (DCM/MeOH=15:1) to afford 2-((1-(3-(4,4-dimethylpiperidin-1-yl)-2-ethyl-7-methyl-1-oxo-1,2-dihydroisoquinolin-5-yl)ethyl)amino)benzoic acid (250 mg, 60%) as a yellow solid. MS: (ES⁺) m/z=462.3 [M+H]⁺.

### Step 4: Preparation of 2-((1-(3-(4,4-dimethylpiperidin-1-yl)-2-ethyl-7-methyl-1-oxo-1,2-dihydroisoquinolin-5-yl)ethyl)amino)benzoic acid (Enantiomers 1 and 2)

A racemic mixture of 2-((1-(3-(4,4-dimethylpiperidin-1-yl)-2-ethyl-7-methyl-1-oxo-1,2-dihydroisoquinolin-5-yl)ethyl)amino)benzoic acid (160 mg, 0.35 mmol) was purified by prep CHIRAL-HPLC (Column: CHIRAL ART Cellulose-SC, 2*25 cm, 5 μm; Mobile Phase A: Hex (0.1% FA)-HPLC, Mobile Phase B: IPA-HPLC; Flow rate: 20 mL/min; Gradient: 5% B to 5% B in 33 min; Wavelengths: 220/254 nm; RT1 (min): 22.187; RT2 (min): 29.177; Sample Solvent: EtOH-HPLC; Injection Volume: 0.5 mL) to afford each enantiomer of 2-((1-(3-(4,4-dimethylpiperidin-1-yl)-2-ethyl-7-methyl-1-oxo-1,2-dihydroisoquinolin-5-yl)ethyl)amino)benzoic acid as a light yellow solid:

2-((1-(3-(4,4-dimethylpiperidin-1-yl)-2-ethyl-7-methyl-1-oxo-1,2-dihydroisoquinolin-5-yl)ethyl)amino)benzoic acid Enantiomer 1 (29 mg, 18%, 99.9% ee). MS: (ES⁺) m/z=462.2 [M+H]⁺. ¹H NMR: (300 MHz, Methanol-d4): δ 7.98 (s, 1H), 7.93-7.76 (m, 1H), 7.52 (d, J=1.9 Hz, 1H), 7.22-7.10 (m, 1H), 6.61 (s, 1H), 6.58-6.47 (m, 1H), 6.41 (d, J=8.4 Hz, 1H), 5.15-5.05 (m, 1H), 4.33-4.17 (m, 2H), 3.00-2.80 (m, 4H), 2.38 (s, 3H), 1.74-1.37 (m, 7H), 1.28 (t, J=6.9 Hz, 3H), 1.04 (s, 6H).

2-((1-(3-(4,4-dimethylpiperidin-1-yl)-2-ethyl-7-methyl-1-oxo-1,2-dihydroisoquinolin-5-yl)ethyl)amino)benzoic acid Enantiomer 2 (29 mg, 17%, 99.3% ee). MS: (ES⁺) m/z=462.2 [M+H]⁺. ¹H NMR: (300 MHz, Methanol-d4): δ 7.98 (s, 1H), 7.93-7.76 (m, 1H), 7.52 (d, J=1.9 Hz, 1H), 7.20-7.10 (m, 1H), 6.61 (s, 1H), 6.58-6.47 (m, 1H), 6.41 (d, J=8.4 Hz, 1H), 5.15-5.05 (m, 1H), 4.34-4.18 (m, 2H), 3.00-2.80 (m, 4H), 2.38 (s, 3H), 1.78-1.36 (m, 7H), 1.28 (t, J=6.9 Hz, 3H), 1.03 (s, 6H).

### Example 30: 2-((1-(2,7-dimethyl-1-oxo-3-((3aR,6aS)-5-(2,2,2-trifluoroethyl)hexahydropyrrolo[3,4-c]pyrrol-2 (1H)-yl)-1,2-dihydroisoquinolin-5-yl)ethyl)amino)benzoic acid (Enantiomer 1)

### Example 31: 2-((1-(2,7-dimethyl-1-oxo-3-((3aR,6aS)-5-(2,2,2-trifluoroethyl)hexahydropyrrolo[3,4-c]pyrrol-2 (1H)-yl)-1,2-dihydroisoquinolin-5-yl)ethyl)amino)benzoic acid (Enantiomer 2)

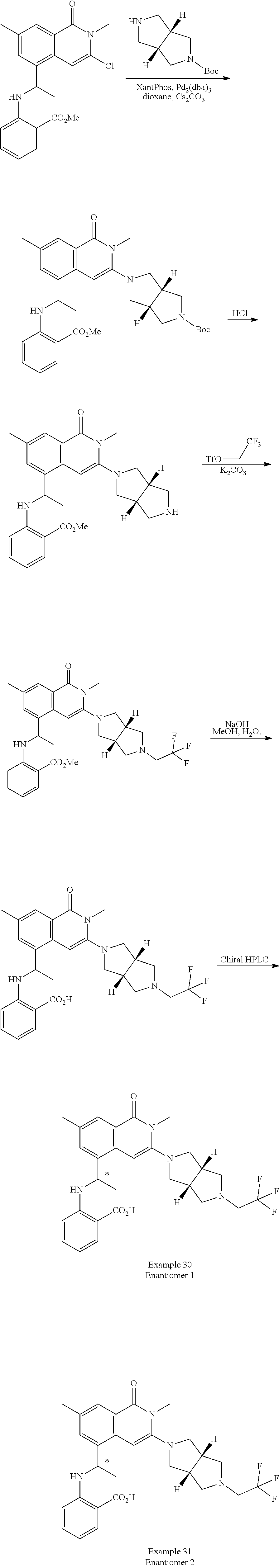

### Step 1: Preparation of tert-butyl (3aR,6aS)-5-(5-(1-((2-(methoxycarbonyl)phenyl)amino)ethyl)-2,7-dimethyl-1-oxo-1,2-dihydroisoquinolin-3-yl)hexahydropyrrolo[3,4-c]pyrrole-2 (1H)-carboxylate

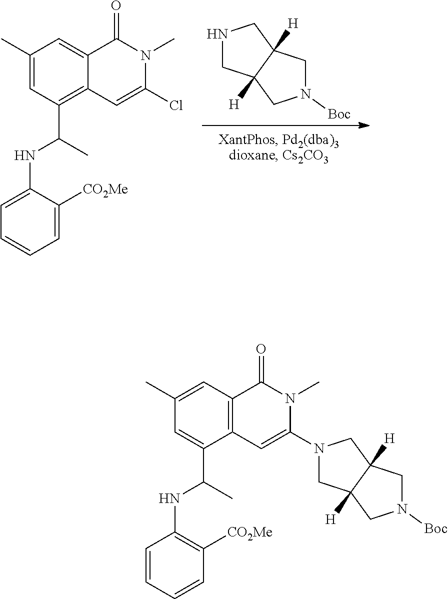

A mixture of methyl 2-((1-(3-chloro-2,7-dimethyl-1-oxo-1,2-dihydroisoquinolin-5-yl)ethyl)amino)benzoate (200 mg, 0.52 mmol), tert-butyl (3aR,6aS)-hexahydro-1H-pyrrolo[3,4-c]pyrrole-2-carboxylate (275 mg, 1.30 mmol), Cs₂CO₃ (509 mg, 1.56 mmol), Xantphos (60 mg, 0.10 mmol) and Pd₂(dba)₃ (47 mg, 0.052 mmol) in 1,4-dioxane (10 mL) was stirred overnight at 100° C. under a nitrogen atmosphere. The reaction mixture was cooled to room temperature, concentrated under reduced pressure, and purified by prep TLC (DCM/MeOH=25:1) to afford tert-butyl (3aR,6aS)-5-(5-(1-((2-(methoxycarbonyl)phenyl)amino)ethyl)-2,7-dimethyl-1-oxo-1,2-dihydroisoquinolin-3-yl)hexahydropyrrolo[3,4-c]pyrrole-2 (1H)-carboxylate (200 mg, 68%) as a yellow solid. MS: (ES⁺) m/z=561.3 [M+H]⁺.

### Step 2: Preparation of methyl 2-((1-(3-((3aR,6aS)-hexahydropyrrolo[3,4-c]pyrrol-2 (1H)-yl)-2,7-dimethyl-1-oxo-1,2-dihydroisoquinolin-5-yl)ethyl)amino)benzoate

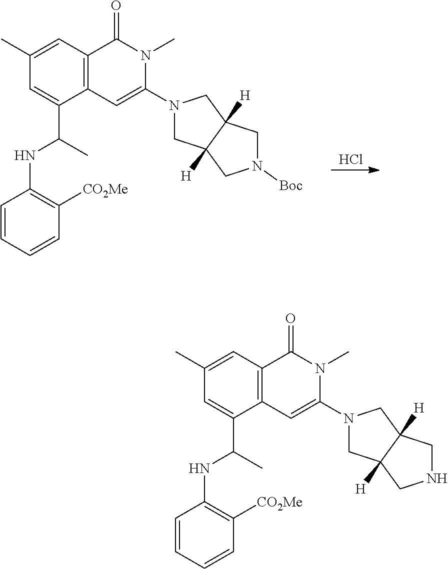 [SMILES: COC(=O)c1ccccc1NC(C)c1cc(C)cc2c(=O)n(C)c(N3C[C@H]4CN(C(=O)OC(C)(C)C)C[C@H]4C3)cc12 ; Cl ; COC(=O)c1ccccc1NC(C)c1cc(C)cc2c(=O)n(C)c(N3C[C@H]4CNC[C@H]4C3)cc12]

A mixture of tert-butyl (3aR,6aS)-5-(5-(1-((2-(methoxycarbonyl)phenyl)amino)ethyl)-2,7-dimethyl-1-oxo-1,2-dihydroisoquinolin-3-yl)hexahydropyrrolo[3,4-c]pyrrole-2 (1H)-carboxylate (200 mg, 0.36 mmol) in a 1:1 mixture of 4 M HCl in 1,4-dioxane and DCM (10 mL) was stirred at room temperature for 1 h under a nitrogen atmosphere. The mixture was concentrated under reduced pressure. The residue was diluted with 5 mL DCM, neutralized with NH₃ in MeOH (7M), and purified by prep TLC (DCM/MeOH=15:1) to afford methyl 2-((1-(3-((3aR,6aS)-hexahydropyrrolo[3,4-c]pyrrol-2 (1H)-yl)-2,7-dimethyl-1-oxo-1,2-dihydroisoquinolin-5-yl)ethyl)amino)benzoate (156 mg, 94%) as a yellow solid. MS: (ES⁺) m/z=461.1 [M+H]⁺.

### Step 3: Preparation of methyl 2-((1-(2,7-dimethyl-1-oxo-3-((3aR,6aS)-5-(2,2,2-trifluoroethyl)hexahydropyrrolo[3,4-c]pyrrol-2 (1H)-yl)-1,2-dihydroisoquinolin-5-yl)ethyl)amino)benzoate

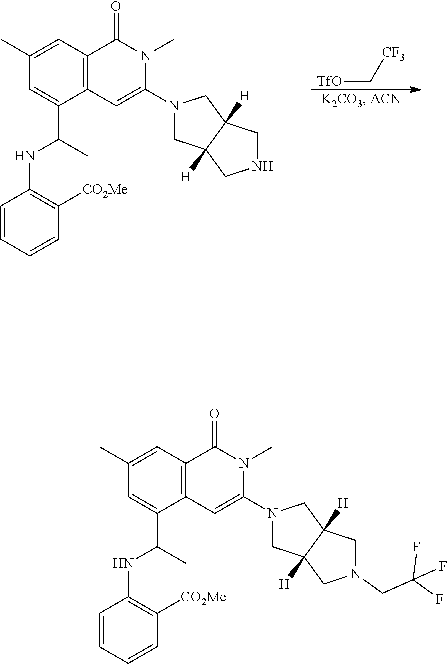

A mixture of methyl 2-((1-(3-((3aR,6aS)-hexahydropyrrolo[3,4-c]pyrrol-2 (1H)-yl)-2,7-dimethyl-1-oxo-1,2-dihydroisoquinolin-5-yl)ethyl)amino)benzoate (156 mg, 0.34 mmol), K₂CO₃ (141 mg, 1.02 mmol) and 2,2,2-trifluoroethyl trifluoromethanesulfonate (0.059 mL, 0.41 mmol) in acetonitrile (20 mL) was stirred overnight at room temperature under a nitrogen atmosphere. The mixture was concentrated under reduced pressure and purified by prep TLC (PE/EA=1:2) to afford methyl 2-((1-(2,7-dimethyl-1-oxo-3-((3aR,6aS)-5-(2,2,2-trifluoroethyl)hexahydropyrrolo[3,4-c]pyrrol-2 (1H)-yl)-1,2-dihydroisoquinolin-5-yl)ethyl)amino)benzoate (110 mg, 59%) as a yellow solid. MS: (ES⁺) m/z=543.2 [M+H]⁺.

### Step 4: Preparation of 2-((1-(2,7-dimethyl-1-oxo-3-((3aR,6aS)-5-(2,2,2-trifluoroethyl)hexahydropyrrolo[3,4-c]pyrrol-2 (1H)-yl)-1,2-dihydroisoquinolin-5-yl)ethyl)amino)benzoic acid

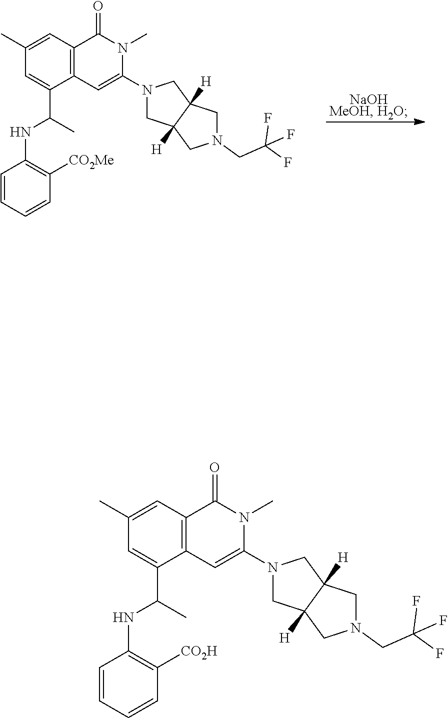 [SMILES: COC(=O)c1ccccc1NC(C)c1cc(C)cc2c(=O)n(C)c(N3C[C@H]4CN(CC(F)(F)F)C[C@H]4C3)cc12 ; "NaOHMeOH, H2O;" ; Cc1cc(C(C)Nc2ccccc2C(=O)O)c2cc(N3C[C@H]4CN(CC(F)(F)F)C[C@H]4C3)n(C)c(=O)c2c1]

A mixture of methyl 2-((1-(2,7-dimethyl-1-oxo-3-((3aR,6aS)-5-(2,2,2-trifluoroethyl)hexahydropyrrolo[3,4-c]pyrrol-2 (1H)-yl)-1,2-dihydroisoquinolin-5-yl)ethyl)amino)benzoate (110 mg, 0.20 mmol), NaOH (121 mg, 3.05 mmol) in methanol (10 mL) and water (1 mL) was stirred overnight at 50° C. under a nitrogen atmosphere. The mixture was adjusted to pH 5-6 with aqueous 1 M HCl. The reaction mixture was diluted with H₂O (30 mL) and extracted with ethyl acetate (3×30 mL). The combined organic layers were washed with brine (2×30 mL), dried over anhydrous sodium sulfate, and then concentrated under reduced pressure. The residue was purified by prep TLC (DCM/MeOH=15:1) and then purified further by prep HPLC (Column: XBridge Prep OBD C18 Column, 30*150 mm, 5 μm; Mobile Phase A: Water (10 mmol/L NH₄HCO₃+0.1% NH₃·H₂O), Mobile Phase B: ACN (Gradient: 24% to 54% in 9 min); Flow rate: 60 mL/min; Detector, UV 254 nm) to afford 2-((1-(2,7-dimethyl-1-oxo-3-((3aR,6aS)-5-(2,2,2-trifluoroethyl)hexahydropyrrolo[3,4-c]pyrrol-2 (1H)-yl)-1,2-dihydroisoquinolin-5-yl)ethyl)amino)benzoic acid (10 mg, 9%) as a white solid. MS: (ES⁺) m/z=529.2 [M+H]⁺.

### Step 5: Preparation of 2-((1-(2,7-dimethyl-1-oxo-3-((3aR,6aS)-5-(2,2,2-trifluoroethyl)hexahydropyrrolo[3,4-c]pyrrol-2 (1H)-yl)-1,2-dihydroisoquinolin-5-yl)ethyl)amino)benzoic acid (Enantiomers 1 and 2)

A racemic mixture of 2-((1-(2,7-dimethyl-1-oxo-3-((3aR,6aS)-5-(2,2,2-trifluoroethyl)hexahydropyrrolo[3,4-c]pyrrol-2 (1H)-yl)-1,2-dihydroisoquinolin-5-yl)ethyl)amino)benzoic acid (30 mg, 0.057 mmol) was separated by prep chiral HPLC with the following conditions (Column: CHIRAL ART Cellulose-SB, 2*25 cm, 5 μm; Mobile Phase A: Hex (0.1% FA)-HPLC, Mobile Phase B: EtOH-HPLC; Flow rate: 20 mL/min; Gradient: 20% B to 20% B in 13.5 min; Wavelengths: 220/254 nm; RT1 (min): 7.385; RT2 (min): 12.31; Sample Solvent: EtOH-HPLC; Injection Volume: 0.5 mL) to afford each enantiomer of 2-((1-(2,7-dimethyl-1-oxo-3-((3aR,6aS)-5-(2,2,2-trifluoroethyl)hexahydropyrrolo[3,4-c]pyrrol-2 (1H)-yl)-1,2-dihydroisoquinolin-5-yl)ethyl)amino)benzoic acid as off-white solids.

2-((1-(2,7-dimethyl-1-oxo-3-((3aR,6aS)-5-(2,2,2-trifluoroethyl)hexahydropyrrolo[3,4-c]pyrrol-2 (1H)-yl)-1,2-dihydroisoquinolin-5-yl)ethyl)amino)benzoic acid Enantiomer 1 (14.4 mg, 46%, 99.9% ee). MS: (ES⁺) m/z=529.2 [M+H]⁺. ¹H NMR: (400 MHz, Methanol-d4): δ 7.96-7.80 (m, 2H), 7.51 (d, J=1.9 Hz, 1H), 7.16-7.10 (m, 1H), 6.56-6.44 (m, 2H), 6.36 (d, J=8.5 Hz, 1H), 5.11-5.03 (m, 1H), 3.64 (s, 3H), 3.23-3.07 (m, 4H), 3.07-2.86 (m, 6H), 2.70-2.55 (m, 2H), 2.36 (s, 3H), 1.62 (d, J=6.6 Hz, 3H).

2-((1-(2,7-dimethyl-1-oxo-3-((3aR,6aS)-5-(2,2,2-trifluoroethyl)hexahydropyrrolo[3,4-c]pyrrol-2 (1H)-yl)-1,2-dihydroisoquinolin-5-yl)ethyl)amino)benzoic acid Enantiomer 2 (14.0 mg, 45%, 99.9% ee). MS: (ES⁺) m/z=529.2 [M+H]⁺. ¹H NMR: ¹H NMR (400 MHz, Methanol-d4): δ 7.96-7.80 (m, 2H), 7.51 (d, J=1.9 Hz, 1H), 7.16-7.05 (m, 1H), 6.54-6.45 (m, 2H), 6.37 (d, J=8.5 Hz, 1H), 5.12-5.01 (m, 1H), 3.64 (s, 3H), 3.23-3.09 (m, 4H), 3.07-2.86 (m, 6H), 2.68-2.63 (m, 2H), 2.36 (s, 3H), 1.63 (d, J=6.6 Hz, 3H).

### Example 32: 2-((1-(2,7-dimethyl-3-(4-(3-methylbutanoyl)piperazin-1-yl)-1-oxo-1,2-dihydroisoquinolin-5-yl)ethyl)amino)benzoic acid (Enantiomer 1)

### Example 33: 2-((1-(2,7-dimethyl-3-(4-(3-methylbutanoyl)piperazin-1-yl)-1-oxo-1,2-dihydroisoquinolin-5-yl)ethyl)amino)benzoic acid (Enantiomer 2)

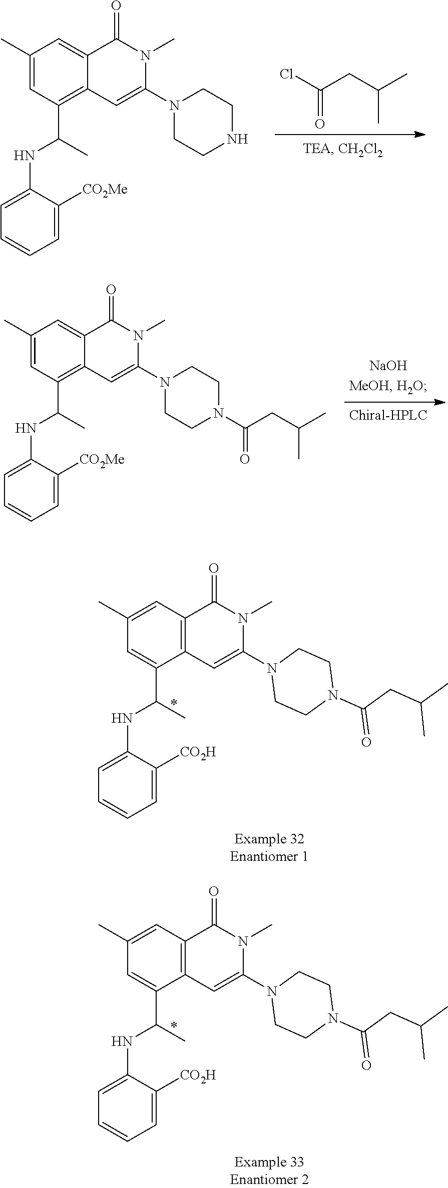

### Step 1: Preparation of methyl 2-((1-(2,7-dimethyl-3-(4-(3-methylbutanoyl)piperazin-1-yl)-1-oxo-1,2-dihydroisoquinolin-5-yl)ethyl)amino)benzoate

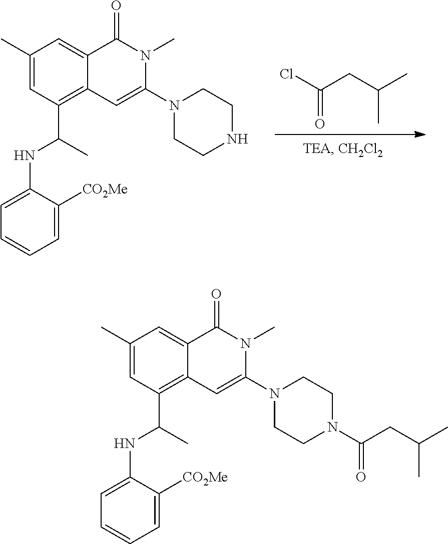

3-Methylbutanoyl chloride (42.5 μL, 0.023 mmol) was added to mixture of methyl 2-((1-(2,7-dimethyl-1-oxo-3-(piperazin-1-yl)-1,2-dihydroisoquinolin-5-yl)ethyl)amino)benzoate (150 mg, 0.35 mmol, prepared in a manner similar to Examples 30 and 31 using tert-butyl piperazine-1-carboxylate in place of tert-butyl (3aR,6aS)-hexahydro-1H-pyrrolo[3,4-c]pyrrole-2-carboxylate in Step 1), CH₂Cl₂ (3 mL), TEA (240 μL, 1.73 mmol) at 0° C. The resulting mixture was stirred for 2 h at room temperature. The resulting mixture was quenched with H₂O (30 mL) and extracted with EtOAc (2×20 mL). The combined organic layers were washed with brine (2×20 mL), dried over anhydrous Na₂SO₄, and then concentrated under reduced pressure. The residue was purified by prep TLC (EA) to afford methyl 2-((1-(2,7-dimethyl-3-(4-(3-methylbutanoyl)piperazin-1-yl)-1-oxo-1,2-dihydroisoquinolin-5-yl)ethyl)amino)benzoate (120 mg, 67%) as a yellow oil. MS: (ES⁺) m/z=519.3 [M+H]⁺.

### Step 2: Preparation of 2-((1-(2,7-dimethyl-3-(4-(3-methylbutanoyl)piperazin-1-yl)-1-oxo-1,2-dihydroisoquinolin-5-yl)ethyl)amino)benzoic acid (Enantiomers 1 and 2)

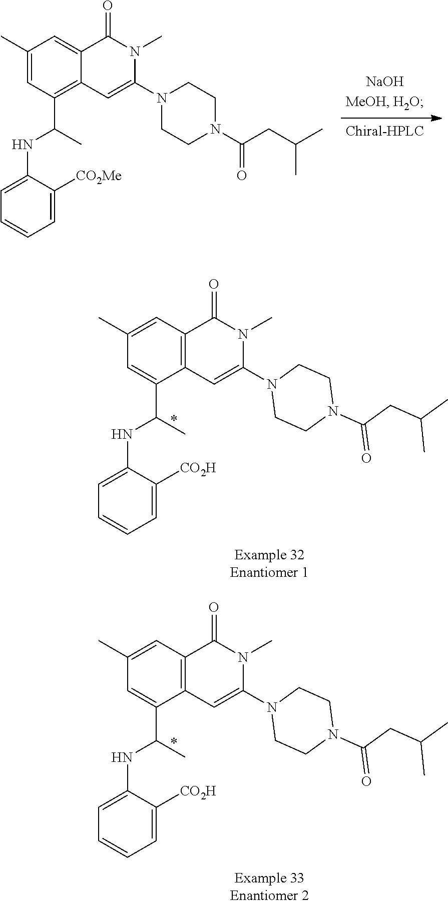

A mixture of methyl 2-((1-(2,7-dimethyl-3-(4-(3-methylbutanoyl)piperazin-1-yl)-1-oxo-1,2-dihydroisoquinolin-5-yl)ethyl)amino)benzoate (120 mg, 0.23 mmol) and NaOH (28 mg, 0.69 mmol) in 5:1 MeOH/H₂O (6 mL) was stirred overnight at 50° C. The mixture was adjusted to pH 5 with an aqueous NaH₂PO₄ solution. The resulting mixture was extracted with EtOAc (2×20 mL). The combined organic layers were washed with brine (2×20 mL), dried over anhydrous Na₂SO₄, and then concentrated under reduced pressure. The residue was purified by reverse phase flash chromatography (Column, C18 silica gel; mobile phase, MeCN in Water (0.1% FA), 5% to 100% gradient in 30 min; detector, UV 254 nm) to afford 2-((1-(2,7-dimethyl-3-(4-(3-methylbutanoyl)piperazin-1-yl)-1-oxo-1,2-dihydroisoquinolin-5-yl)ethyl)amino)benzoic acid (100 mg, 85%) as a yellow solid. MS: (ES⁺) m/z=505.3 [M+H]⁺.

A racemic mixture of 2-((1-(2,7-dimethyl-3-(4-(3-methylbutanoyl)piperazin-1-yl)-1-oxo-1,2-dihydroisoquinolin-5-yl)ethyl)amino)benzoic acid (100 mg, 0.198 mmol) was purified by prep chiral HPLC (Column: CHIRAL Cellulose-SB, 4.6*100 mm, 3 μm; Mobile Phase: Hex (0.1% FA): EtOH=70:30; Flow rate: 1.0 mL/min) to afford each enantiomer of 2-((1-(2,7-dimethyl-3-(4-(3-methylbutanoyl)piperazin-1-yl)-1-oxo-1,2-dihydroisoquinolin-5-yl)ethyl)amino)benzoic acid as a white solid:

2-((1-(2,7-dimethyl-3-(4-(3-methylbutanoyl)piperazin-1-yl)-1-oxo-1,2-dihydroisoquinolin-5-yl)ethyl)amino)benzoic acid Enantiomer 1 (24.2 mg, 24%, >99.9% ee) as a white solid. MS: (ES⁺) m/z=505.3 [M+H]⁺. ¹H NMR (400 MHz, DMSO-d6) δ 12.76 (s, 1H), 8.43 (s, 1H), 7.90-7.88 (m, 1H), 7.85-7.80 (m, 1H), 7.42 (d, J=1.8 Hz, 1H), 7.25-7.18 (m, 1H), 6.55-6.52 (m, 1H), 6.42-6.33 (m, 2H), 5.22-5.14 (m, 1H), 4.46-3.92 (m, 1H), 3.54 (s, 3H), 3.20-2.84 (m, 3H), 2.34 (s, 3H), 2.29-2.26 (m, 5H), 2.05-2.02 (m, 1H), 1.53 (d, J=6.5 Hz, 3H), 0.93 (d, J=6.6 Hz, 6H).

2-((1-(2,7-dimethyl-3-(4-(3-methylbutanoyl)piperazin-1-yl)-1-oxo-1,2-dihydroisoquinolin-5-yl)ethyl)amino)benzoic acid Enantiomer 2 (24.0 mg, 24%, >99.9% ee) as a white solid. MS: (ES⁺) m/z=505.3 [M+H]⁺. ¹H NMR: (400 MHz, DMSO-d6) δ 12.72 (s, 1H), 8.48 (s, 1H), 7.90-7.88 (m, 1H), 7.85-7.80 (m, 1H), 7.42 (d, J=1.9 Hz, 1H), 7.25-7.18 (m, 1H), 6.55-6.52 (m, 1H), 6.42-6.33 (m, 2H), 5.22-5.14 (m, 1H), 4.46-3.92 (m, 1H), 3.54 (s, 3H), 3.20-2.84 (m, 3H), 2.34 (s, 3H), 2.29-2.26 (m, 5H), 2.05-2.02 (m, 1H), 1.53 (d, J=6.5 Hz, 3H), 0.93 (d, J=6.6 Hz, 6H).

### Example 34: (R)-2-((1-(3-(benzylthio)-2,7-dimethyl-1-oxo-1,2-dihydroisoquinolin-5-yl)ethyl)amino)benzoic acid

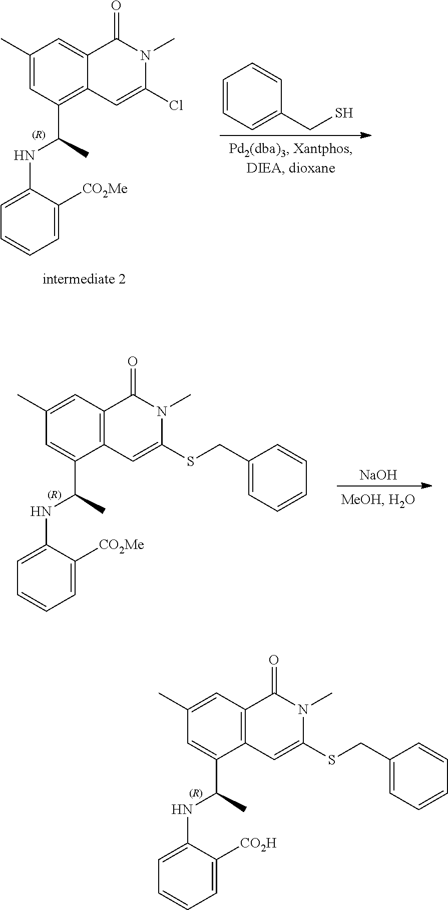

### Step 1: Preparation of methyl (R)-2-((1-(3-(benzylthio)-2,7-dimethyl-1-oxo-1,2-dihydroisoquinolin-5-yl)ethyl)amino)benzoate

To a stirred solution of methyl (R)-2-((1-(3-chloro-2,7-dimethyl-1-oxo-1,2-dihydroisoquinolin-5-yl)ethyl)amino)benzoate (100 mg, 0.26 mmol) and benzyl mercaptan (32 mg, 0.26 mmol) in dioxane (2 mL) were added DIEA (101 mg, 0.78 mmol), Xantphos (30 mg, 0.052 mmol) and Pd₂(dba)₃ (24 mg, 0.026 mmol). The resulting mixture was stirred for 12 h at 100° C. under a N₂ atmosphere. The resulting mixture was diluted with H₂O (20 mL) and extracted with EA (3×30 mL). The combined organic layers were washed with brine (50 mL), dried over anhydrous Na₂SO₄, and then concentrated under reduced pressure. The residue was purified by prep TLC (PE/EA=3:1) to afford methyl (R)-2-((1-(3-(benzylthio)-2,7-dimethyl-1-oxo-1,2-dihydroisoquinolin-5-yl)ethyl)amino)benzoate (110 mg, 89%) as a yellow solid. MS (ES⁺) m/z=473.1 [M+H]⁺.

### Step 2: Preparation of (R)-2-((1-(3-(benzylthio)-2,7-dimethyl-1-oxo-1,2-dihydroisoquinolin-5-yl)ethyl)amino)benzoic acid

To a stirred solution of methyl (R)-2-((1-(3-(benzylthio)-2,7-dimethyl-1-oxo-1,2-dihydroisoquinolin-5-yl)ethyl)amino)benzoate (110 mg, 0.23 mmol) in MeOH (4 mL) was added NaOH (93 mg, 2.33 mmol) in H₂O (2 mL). The resulting mixture was stirred for 12 h at 50° C. The mixture was adjusted to pH 5 with 2 M aqueous HCl. The resulting mixture was diluted with H₂O (30 mL and extracted with DCM (3×50 mL). The combined organic layers were washed with brine (50 mL), dried over anhydrous Na₂SO₄, and then concentrated under reduced pressure. The crude product was purified by prep HPLC with the following conditions (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 26% B to 46% B in 8.5 min, 46% B; Wave Length: 254) to afford (R)-2-((1-(3-(benzylthio)-2,7-dimethyl-1-oxo-1,2-dihydroisoquinolin-5-yl)ethyl)amino)benzoic acid (59.4 mg, 55%) as an off-white solid. MS (ES⁺) m/z=481.1 [M+Na]⁺. ¹H NMR: (400 MHz, Methanol-d4) δ 8.0-7.92 (m, 1H), 7.92-7.85 (m, 1H), 7.53 (d, J=1.9 Hz, 1H), 7.32-7.14 (m, 5H), 7.10-7.0 (m, 1H), 6.90 (s, 1H), 6.55-6.45 (m, 1H), 6.15-6.1 (m, 1H), 4.95-4.85 (m, 1H), 4.18 (s, 2H), 3.75 (s, 3H), 2.34 (s, 3H), 1.43 (d, J=6.7 Hz, 3H).

### Example 35: 2-((1-(2,7-dimethyl-1-oxo-3-(phenylethynyl)-1,2-dihydroisoquinolin-5-yl)ethyl)amino)benzoic acid (Enantiomer 1)

### Example 36: 2-((1-(2,7-dimethyl-1-oxo-3-(phenylethynyl)-1,2-dihydroisoquinolin-5-yl)ethyl)amino)benzoic acid (Enantiomer 2)

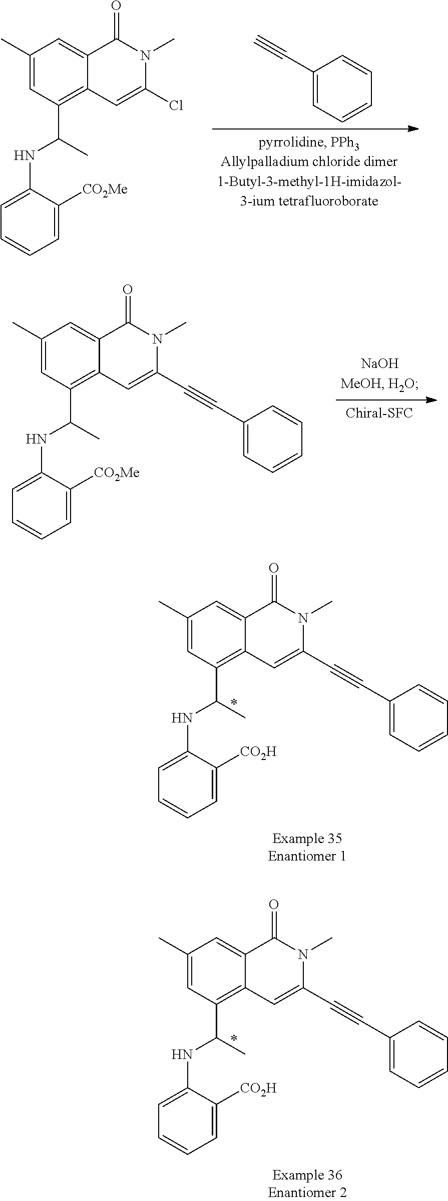

### Step 1: Preparation of methyl 2-((1-(2,7-dimethyl-1-oxo-3-(phenylethynyl)-1,2-dihydroisoquinolin-5-yl)ethyl)amino)benzoate

To a stirred solution of methyl 2-((1-(3-chloro-2,7-dimethyl-1-oxo-1,2-dihydroisoquinolin-5-yl)ethyl)amino)benzoate (450 mg, 1.17 mmol) and phenylacetylene (239 mg, 2.34 mmol) in 1-butyl-3-methyl-1H-imidazol-3-ium tetrafluoroborate (3 mL) was added pyrrolidine (166 mg, 2.34 mmol) at room temperature under a nitrogen atmosphere. To the above mixture was added PPh₃ (31 mg, 0.12 mmol) and allylpalladium chloride dimer (43 mg, 0.12 mmol) at room temperature. The resulting mixture was stirred for 2 h at 100° C. under a nitrogen atmosphere. The resulting mixture was quenched with H₂O (20 mL) and extracted with EA (3×10 mL). The combined organic layers were washed with brine (1×10 mL) and dried over anhydrous Na₂SO₄. After filtration, the filtrate was concentrated under reduced pressure. The residue was purified by silica gel column chromatography, eluted with PE/EA (8:2), to afford methyl 2-((1-(2,7-dimethyl-1-oxo-3-(phenylethynyl)-1,2-dihydroisoquinolin-5-yl)ethyl)amino)benzoate (230 mg, 44% yield) as a light yellow solid. LCMS m/z 451.3 [M+H]⁺.

### Step 2: Preparation of 2-((1-(2,7-dimethyl-1-oxo-3-(phenylethynyl)-1,2-dihydroisoquinolin-5-yl)ethyl)amino)benzoic acid (Enantiomers 1 and 2)

To a stirred mixture of methyl 2-((1-(2,7-dimethyl-1-oxo-3-(phenylethynyl)-1,2-dihydroisoquinolin-5-yl)ethyl)amino)benzoate (230 mg, 0.51 mmol) in MeOH (5 mL) and H₂O (1 mL) was added NaOH (408 mg, 10.2 mmol) at room temperature under a nitrogen atmosphere. The resulting mixture was stirred for 1 h at 70° C. under a nitrogen atmosphere. The residue was adjusted to pH 6 with aqueous 1 N HCl. The residue was purified by reversed-phase flash chromatography to give 2-((1-(2,7-dimethyl-1-oxo-3-(phenylethynyl)-1,2-dihydroisoquinolin-5-yl)ethyl)amino)benzoic acid (110 mg, 49% yield) as an off-white solid. LCMS m/z 437.4 [M+H]⁺.

The racemic product 2-((1-(2,7-dimethyl-1-oxo-3-(phenylethynyl)-1,2-dihydroisoquinolin-5-yl)ethyl)amino)benzoic acid (108 mg) was separated into the individual enantiomers by chiral-SFC to give 2-((1-(2,7-dimethyl-1-oxo-3-(phenylethynyl)-1,2-dihydroisoquinolin-5-yl)ethyl)amino)benzoic acid (Enantiomer 1, 29 mg, 27% yield) as a white solid, LCMS m/z 437.1 [M+H]⁺, ¹H NMR (300 MHz, Chloroform-d) δ 8.28-8.17 (m, 2H), 8.01 (dd, J=8.1, 1.7 Hz, 1H), 7.65-7.59 (m, 2H), 7.54 (d, J=1.8 Hz, 1H), 7.48-7.36 (m, 3H), 7.25-7.13 (m, 2H), 6.60 (ddd, J=8.1, 7.1, 1.0 Hz, 1H), 6.27 (d, J=8.5 Hz, 1H), 5.15-5.0 (m, 1H), 3.86 (s, 3H), 2.42 (s, 3H), 1.68 (d, J=6.7 Hz, 3H); and 2-((1-(2,7-dimethyl-1-oxo-3-(phenylethynyl)-1,2-dihydroisoquinolin-5-yl)ethyl)amino)benzoic acid (Enantiomer 2, 28 mg, 26% yield) as a white solid. LCMS m/z 437.0 [M+H]⁺, ¹H NMR (300 MHz, Chloroform-d) δ 8.25-8.11 (m, 2H), 8.01 (dd, J=8.1, 1.7 Hz, 1H), 7.69-7.58 (m, 2H), 7.57-7.53 (m, 1H), 7.48-7.35 (m, 3H), 7.25-7.13 (m, 2H), 6.66-6.55 (m, 1H), 6.27 (d, J=8.5 Hz, 1H), 5.15-5.0 (m, 1H), 3.86 (s, 3H), 2.42 (s, 3H), 1.68 (d, J=6.7 Hz, 3H).

### Example 37: 2-((1-(2,7-dimethyl-1-oxo-3-(4-(2,2,2-trifluoroethyl)piperazin-1-yl)-1,2-dihydroisoquinolin-5-yl)-2-fluoroethyl)amino)benzoic acid (Enantiomer 1)

### Example 38: 2-((1-(2,7-dimethyl-1-oxo-3-(4-(2,2,2-trifluoroethyl)piperazin-1-yl)-1,2-dihydroisoquinolin-5-yl)-2-fluoroethyl)amino)benzoic acid (Enantiomer 1)

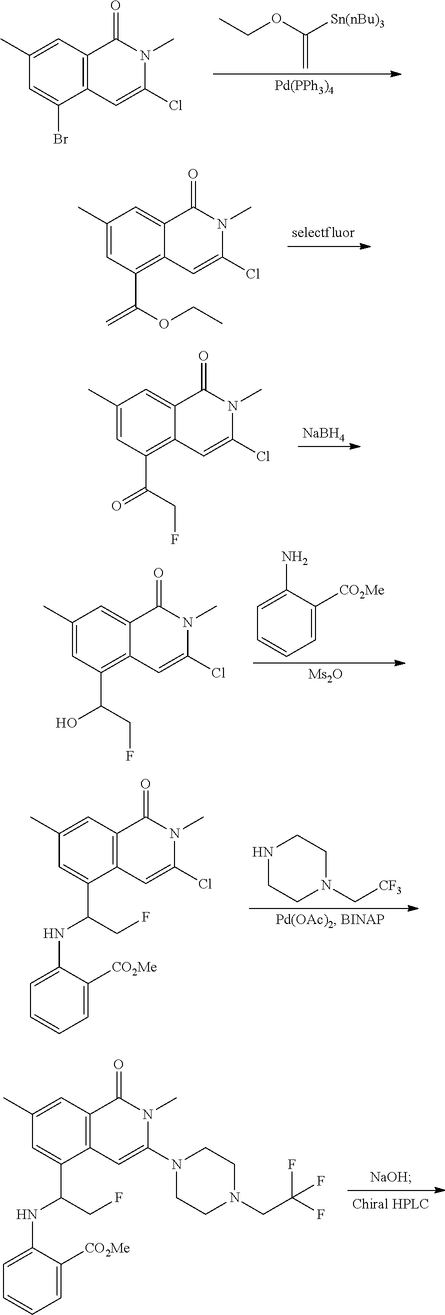

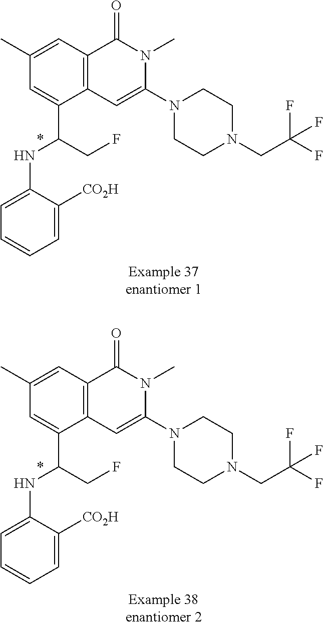 [SMILES: Cc1cc(C(CF)Nc2ccccc2C(=O)O)c2cc(N3CCN(CC(F)(F)F)CC3)n(C)c(=O)c2c1 ; Cc1cc(C(CF)Nc2ccccc2C(=O)O)c2cc(N3CCN(CC(F)(F)F)CC3)n(C)c(=O)c2c1]

### Step 1: Preparation of 3-chloro-5-(1-ethoxyvinyl)-2,7-dimethylisoquinolin-1 (2H)-one

A solution of 5-bromo-3-chloro-2,7-dimethylisoquinolin-1-one (5 g, 17.4 mmol) in dioxane (80 mL) was treated with tributyl(1-ethoxyethenyl)stannane (5.67 g, 15.7 mmol) and Pd(PPh₃)₄ (2.02 g, 1.7 mmol) in portions at room temperature. The resulting mixture was stirred overnight at 90° C. under a nitrogen atmosphere. The resulting mixture was concentrated under reduced pressure. The residue was purified by silica gel column chromatography, eluted with PE/EA (1:1), to afford 3-chloro-5-(1-ethoxyethenyl)-2,7-dimethylisoquinolin-1-one (4.2 g, 87% yield) as a yellow solid. MS: (ES⁺) m/z=278.1 [M+H]⁺.

### Step 2: Preparation of 3-chloro-5-(2-fluoroacetyl)-2,7-dimethylisoquinolin-1 (2H)-one

To a stirred solution of Selectfluor™ (1.42 g, 4 mmol) in ACN (10 mL) and H₂O (5 mL) were added 3-chloro-5-(1-ethoxyethenyl)-2,7-dimethylisoquinolin-1-one (740 mg, 2.7 mmol) in CH₃CN (10 mL) dropwise at 0° C. The resulting mixture was stirred for 1 h at room temperature. The reaction was then quenched with a saturated aqueous NaHCO₃ solution (10 mL) at room temperature. The resulting mixture was extracted with EA (3×50 mL). The combined organic layers were washed with brine (2×200 mL), dried over anhydrous Na₂SO₄, and filtered. The filtrate was concentrated under reduced pressure. The residue was purified by silica gel column chromatography, eluted with PE/EA (1:1), to afford 3-chloro-5-(2-fluoroacetyl)-2,7-dimethylisoquinolin-1-one (500 mg, 70% yield) as a light yellow solid. MS: (ES⁺) m/z=268.1 [M+H]⁺.

### Step 3: Preparation of 3-chloro-5-(2-fluoro-1-hydroxyethyl)-2,7-dimethylisoquinolin-1 (2H)-one

To a stirred solution of 3-chloro-5-(2-fluoroacetyl)-2,7-dimethylisoquinolin-1-one (500 mg, 1.9 mmol) in MeOH (6 mL) was slowly added NaBH₄ (141 mg, 3.7 mmol) portionwise at 0° C. The resulting solution was stirred for 1 h at room temperature. The reaction was quenched by the addition of saturated aqueous ammonium chloride solution (20 mL) at room temperature. The resulting mixture was extracted with EA (3×30 mL). The combined organic layers were washed with brine (3×20 mL), dried over anhydrous Na₂SO₄, and filtered. The filtrate was concentrated under reduced pressure. The residue was purified by silica gel column chromatography, eluted with PE/EA (1:1), to afford 3-chloro-5-(2-fluoro-1-hydroxyethyl)-2,7-dimethylisoquinolin-1-one (198 mg, 39% yield) as a white solid. MS: (ES⁺) m/z=270.1 [M+H]⁺.

### Step 4: Preparation of methyl 2-((1-(3-chloro-2,7-dimethyl-1-oxo-1,2-dihydroisoquinolin-5-yl)-2-fluoroethyl)amino)benzoate

To a stirred solution/mixture of 3-chloro-5-(2-fluoro-1-hydroxyethyl)-2,7-dimethylisoquinolin-1-one (180 mg, 0.67 mmol) in DCM was added TEA (405 mg, 4 mmol) and methanesulfonic anhydride (486 mg, 2.8 mmol) successively at 0° C. The resulting solution was stirred for 1 h at 0° C. To the solution was added methyl anthranilate (486 mg, 3.2 mmol). The resulting solution was stirred overnight at room temperature. The residue was purified by silica gel column chromatography, eluted with PE/EA (1:1), to afford methyl 2-((1-(3-chloro-2,7-dimethyl-1-oxo-1,2-dihydroisoquinolin-5-yl)-2-fluoroethyl)amino)benzoate (170 mg, 63% yield) as a light yellow solid. MS: (ES⁺) m/z=403.2 [M+H]⁺.

### Step 5: Preparation of methyl 2-((1-(2,7-dimethyl-1-oxo-3-(4-(2,2,2-trifluoroethyl)piperazin-1-yl)-1,2-dihydroisoquinolin-5-yl)-2-fluoroethyl)amino)benzoate

To a stirred solution of methyl 2-((1-(3-chloro-2,7-dimethyl-1-oxo-1,2-dihydroisoquinolin-5-yl)-2-fluoroethyl)amino)benzoate (170 mg, 0.42 mmol) in 1,4-dioxane (10 mL) was added 1-(2,2,2-trifluoroethyl)piperazine (177 mg, 1.1 mmol), Cs₂CO₃ (550 mg, 1.7 mmol), BINAP (26 mg, 0.04 mmol) and Pd(OAc)₂ (9 mg, 0.042 mmol) in portions at room temperature under a nitrogen atmosphere. The resulting solution was stirred overnight at 100° C. under a nitrogen atmosphere. The reaction was quenched by the addition of water (20 mL) at room temperature. The aqueous layer was extracted with EA (3×20 mL). The combined organic layers were dried over anhydrous Na₂SO₄ and then filtered. The filtrate was then concentrated under reduced pressure. The residue was purified by silica gel column chromatography, eluted with PE/EA (1:1), to afford methyl 2-((1-(2,7-dimethyl-1-oxo-3-(4-(2,2,2-trifluoroethyl)piperazin-1-yl)-1,2-dihydroisoquinolin-5-yl)-2-fluoroethyl)amino)benzoate (173 mg, 77% yield) as a light yellow oil. MS: (ES⁺) m/z=535.3 [M+H]⁺.

### Step 6: Preparation of 2-((1-(2,7-dimethyl-1-oxo-3-(4-(2,2,2-trifluoroethyl)piperazin-1-yl)-1,2-dihydroisoquinolin-5-yl)-2-fluoroethyl)amino)benzoic acid (Enantiomers 1 and 2)

To a stirred solution of methyl 2-((1-(2,7-dimethyl-1-oxo-3-(4-(2,2,2-trifluoroethyl)piperazin-1-yl)-1,2-dihydroisoquinolin-5-yl)-2-fluoroethyl)amino)benzoate (163 mg, 0.31 mmol) in MeOH (2 mL) and water (2 mL) was added NaOH (125 mg, 3.1 mmol) in portions at room temperature. The resulting mixture was stirred for 1 h at 50° C. under a nitrogen atmosphere. The mixture was neutralized to pH 6 with aqueous HCl (1 N) and the mixture was extracted with EA (3×30 mL). The organic phases were then washed with brine (2×30 mL). The combined organic phases were dried with Na₂SO₄, filtered and concentrated. The residue was purified by reversed-phase flash chromatography to give 2-((1-(2,7-dimethyl-1-oxo-3-(4-(2,2,2-trifluoroethyl)piperazin-1-yl)-1,2-dihydroisoquinolin-5-yl)-2-fluoroethyl)amino)benzoic acid (110 mg, 67% yield) as a white solid. MS: (ES⁺) m/z=521.3 [M+H]⁺.

The racemic product 2-((1-(2,7-dimethyl-1-oxo-3-(4-(2,2,2-trifluoroethyl)piperazin-1-yl)-1,2-dihydroisoquinolin-5-yl)-2-fluoroethyl)amino)benzoic acid (110 mg) was separated by chiral-HPLC with the following conditions (Column: CHIRALCEL OD-3 4.6*50 mm, 3 μm; Mobile Phase A: Hexane (0.1% FA): EtOH=90:10; Gradient: isocratic) to afford 2-((1-(2,7-dimethyl-1-oxo-3-(4-(2,2,2-trifluoroethyl)piperazin-1-yl)-1,2-dihydroisoquinolin-5-yl)-2-fluoroethyl)amino)benzoic acid (Enantiomer 1, 38 mg, 32% yield, 99% ee) as a light yellow solid, ¹H NMR (300 MHz, Methanol-d4) δ 8.05 (s, 1H), 7.96-7.92 (m, 1H), 7.62-7.59 (m, 1H), 7.23-7.18 (m, 1H), 6.68-6.55 (m, 1H), 6.49 (m, 2H), 5.42-5.38 (m, 1H), 4.97-4.80 (m, 2H), 3.65 (s, 3H), 3.19-3.05 (m, 2H), 2.89-2.85 (m, 8H), 2.41 (s, 3H), MS: (ES⁺) m/z=521.1 [M+H]⁺; and 2-((1-(2,7-dimethyl-1-oxo-3-(4-(2,2,2-trifluoroethyl)piperazin-1-yl)-1,2-dihydroisoquinolin-5-yl)-2-fluoroethyl)amino)benzoic acid (Enantiomer 2, 34 mg, 31% yield, >99% ee) as a white solid, ¹H NMR (300 MHz, Methanol-d4) δ 8.05 (s, 1H), 7.96-7.92 (m, 1H), 7.62-7.59 (m, 1H), 7.23-7.18 (m, 1H), 6.68-6.55 (m, 1H), 6.49 (m, 2H), 5.42-5.38 (m, 1H), 4.97-4.80 (m, 2H), 3.65 (s, 3H), 3.22-3.17 (m, 2H), 2.95-2.84 (m, 8H), 2.41 (s, 3H), MS: (ES⁺) m/z=521.1 [M+H]⁺.

### Example 39: (R)-2-fluoro-6-((1-(7-fluoro-2-methyl-1-oxo-3-(3-(trifluoromethyl)bicyclo[1.1.1]pentan-1-yl)-1,2-dihydroisoquinolin-5-yl)ethyl)amino)benzoic acid

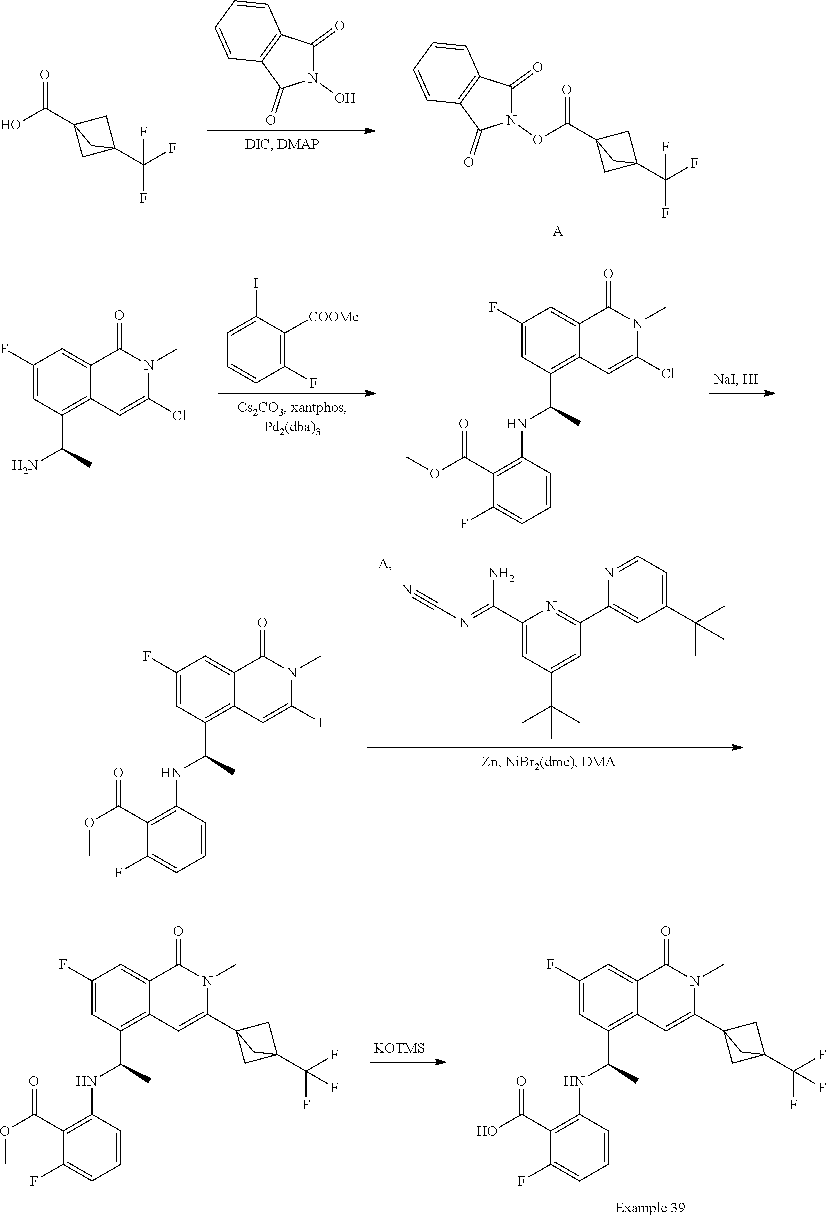

### Step 1: Preparation of 1,3-dioxoisoindolin-2-yl 3-(trifluoromethyl)bicyclo[1.1.1]pentane-1-carboxylate

To a stirred solution of 3-(trifluoromethyl)bicyclo[1.1.1]pentane-1-carboxylic acid (1.0 g, 5.6 mmol), DMAP (102 mg, 0.83 mmol), and N-hydroxyphthalimide (1.0 g, 6.0 mmol) in DCM (20 mL) was added N,N′-diisopropylcarbodiimide (0.95 mL, 6.1 mmol) dropwise at 0° C. under an argon atmosphere. The resulting mixture was stirred overnight. The resulting mixture was filtered and the filter cake was washed with DCM (3×30 mL). The filtrate was concentrated under reduced pressure. The resulting mixture was dissolved with EtOAc (50 mL). The combined organic layers were washed with aqueous Na₂CO₃ (3×50 mL) and aqueous 1 N HCl (3×50 mL) and then dried over anhydrous Na₂SO₄. After filtration, the filtrate was concentrated under reduced pressure to afford 1,3-dioxoisoindol-2-yl 3-(trifluoromethyl)bicyclo[1.1.1]pentane-1-carboxylate (1.6 g, 89% yield) as an off-white solid. ¹H NMR (400 MHz, Chloroform-d) δ 7.93-7.87 (m, 2H), 7.83-7.78 (m, 2H), 2.50 (s, 6H).

### Step 2: Preparation of methyl (R)-2-((1-(3-chloro-7-fluoro-2-methyl-1-oxo-1,2-dihydroisoquinolin-5-yl)ethyl)amino)-6-fluorobenzoate

Into a 100 mL round-bottom flask were added 5-[(1R)-1-aminoethyl]-3-chloro-7-fluoro-2-methylisoquinolin-1-one (1.0 g, 3.9 mmol), dioxane (30 mL), methyl 2-fluoro-6-iodobenzoate (0.6 mL, 3.9 mmol), Cs₂CO₃ (3.84 g, 11.8 mmol), Xantphos (454 mg, 0.78 mmol) and Pd₂(dba)₃ (360 mg, 0.39 mmol) at room temperature. The resulting mixture was stirred for 3 h at 90° C. under an argon atmosphere. The resulting mixture was poured into water (60 mL). The resulting mixture was extracted with EtOAc (3×60 mL). The combined organic layers were washed with brine and then dried over anhydrous Na₂SO₄. After filtration, the filtrate was concentrated under reduced pressure. The residue was purified by silica gel column chromatography to afford methyl (R)-2-((1-(3-chloro-7-fluoro-2-methyl-1-oxo-1,2-dihydroisoquinolin-5-yl)ethyl)amino)-6-fluorobenzoate (922 mg, 58% yield) as a light yellow solid. LCMS (ES⁺) m/z=407.0 [M+H]⁺.

### Step 3: Preparation of methyl (R)-2-fluoro-6-((1-(7-fluoro-3-iodo-2-methyl-1-oxo-1,2-dihydroisoquinolin-5-yl)ethyl)amino)benzoate

Into a 40 mL vial were added methyl (R)-2-((1-(3-chloro-7-fluoro-2-methyl-1-oxo-1,2-dihydroisoquinolin-5-yl)ethyl)amino)-6-fluorobenzoate (729 mg, 1.8 mmol), HI (20 mL) and NaI (1.34 g, 9 mmol) at room temperature. The resulting mixture was stirred for overnight at room temperature under an argon atmosphere. The mixture was adjusted to pH 7 by the addition of a saturated aqueous NaHCO₃ solution (50 mL). The resulting mixture was extracted with EtOAc (3×50 mL). The combined organic layers were washed with brine and then dried over anhydrous Na₂SO₄. After filtration, the filtrate was concentrated under reduced pressure. The residue was purified by silica gel column chromatography to afford methyl (R)-2-fluoro-6-((1-(7-fluoro-3-iodo-2-methyl-1-oxo-1,2-dihydroisoquinolin-5-yl)ethyl)amino)benzoate (526 mg, 63% yield) as a yellow solid. ¹H NMR (400 MHz, DMSO-d6) δ 7.79-7.71 (m, 2H), 7.66 (s, 1H), 7.50-7.43 (m, 1H), 7.22-7.14 (m, 1H), 6.43-6.36 (m, 1H), 6.11 (d, J=8.6 Hz, 1H), 5.30-5.22 (m, 1H), 3.90 (s, 3H), 3.77 (s, 3H), 1.52 (d, J=6.4 Hz, 3H).

### Step 4: Preparation of methyl (R)-2-fluoro-6-((1-(7-fluoro-2-methyl-1-oxo-3-(3-(trifluoromethyl)bicyclo[1.1.1]pentan-1-yl)-1,2-dihydroisoquinolin-5-yl)ethyl)amino)benzoate

Into a 20 mL vial were added methyl (R)-2-fluoro-6-((1-(7-fluoro-3-iodo-2-methyl-1-oxo-1,2-dihydroisoquinolin-5-yl)ethyl)amino)benzoate (200 mg, 0.4 mmol), DMA (5 mL), 1,3-dioxoisoindol-2-yl 3-(trifluoromethyl)bicyclo[1.1.1]pentane-1-carboxylate (261 mg, 0.8 mmol), (Z)-4,4′-di-tert-butyl-N′-cyano-[2,2′-bipyridine]-6-carboximidamide (27 mg, 0.08 mmol), Zn (105 mg, 1.6 mmol) and NiBr₂·DME (14 mg, 0.04 mmol) at room temperature. The resulting mixture was stirred for overnight at 50° C. under an argon atmosphere. The reaction contents were then poured into water (60 mL) and the resulting mixture was extracted with EtOAc (3×60 mL). The combined organic layers were washed with brine and then dried over anhydrous Na₂SO₄. After filtration, the filtrate was concentrated under reduced pressure. The residue was purified by silica gel column chromatography to afford methyl (R)-2-fluoro-6-((1-(7-fluoro-2-methyl-1-oxo-3-(3-(trifluoromethyl)bicyclo[1.1.1]pentan-1-yl)-1,2-dihydroisoquinolin-5-yl)ethyl)amino)benzoate (52 mg, 26% yield) as an off-white solid. LCMS (ES⁺) m/z=507.3 [M+H]⁺.

### Step 5: Preparation of (R)-2-fluoro-6-((1-(7-fluoro-2-methyl-1-oxo-3-(3-(trifluoromethyl)bicyclo[1.1.1]pentan-1-yl)-1,2-dihydroisoquinolin-5-yl)ethyl)amino)benzoic acid

Into an 8 mL vial were added methyl (R)-2-fluoro-6-((1-(7-fluoro-2-methyl-1-oxo-3-(3-(trifluoromethyl)bicyclo[1.1.1]pentan-1-yl)-1,2-dihydroisoquinolin-5-yl)ethyl)amino)benzoate (52 mg, 0.1 mmol), THF (2 mL) and trimethyl(potassiooxy)silane (40 mg, 0.3 mmol) at room temperature. The resulting mixture was stirred for 3 h at 70° C. under an argon atmosphere. The resulting mixture was then poured into water (10 mL) and extracted with EtOAc (3×10 mL). The combined organic layers were washed with brine and dried over anhydrous Na₂SO₄. After filtration, the filtrate was concentrated under reduced pressure. The crude product was purified by prep-HPLC to afford (R)-2-fluoro-6-((1-(7-fluoro-2-methyl-1-oxo-3-(3-(trifluoromethyl)bicyclo[1.1.1]pentan-1-yl)-1,2-dihydroisoquinolin-5-yl)ethyl)amino)benzoic acid (5.5 mg, 11% yield) as a white solid. LCMS (ES⁺) m/z=493.1 [M+H]⁺. ¹H NMR (400 MHz, DMSO-d₆) δ 8.08 (d, J=5.9 Hz, 1H), 7.80-7.74 (m, 1H), 7.50-7.47 (m, 1H), 7.20-7.14 (m, 1H), 6.68 (s, 1H), 6.40-6.33 (m, 1H), 6.16 (d, J=8.6 Hz, 1H), 5.29-5.21 (m, 1H), 3.62 (s, 3H), 2.52 (s, 6H), 1.56 (d, J=6.6 Hz, 3H).

### Example 40: (R)-2-((1-(3-isobutoxy-2,7-dimethyl-1-oxo-1,2-dihydroisoquinolin-5-yl)ethyl)amino)benzoic acid

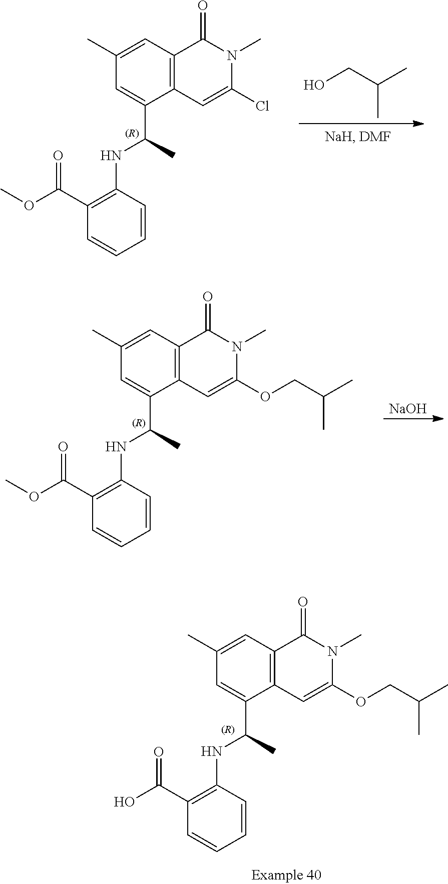

### Step 1: Preparation of methyl (R)-2-((1-(3-isobutoxy-2,7-dimethyl-1-oxo-1,2-dihydroisoquinolin-5-yl)ethyl)amino)benzoate

A stirred solution of methyl (R)-2-((1-(3-chloro-2,7-dimethyl-1-oxo-1,2-dihydroisoquinolin-5-yl)ethyl)amino)benzoate (200 mg, 0.52 mmol) in DMF (10 mL) was treated with NaH (37 mg, 1.6 mmol) and stirred for 15 min at 0° C. under a nitrogen atmosphere. Isobutanol (193 mg, 2.6 mmol) was then added in portions at 0° C. The resulting mixture was stirred for 2 h at 80° C. under a nitrogen atmosphere. The reaction was quenched by the addition of water (20 mL) at room temperature. The resulting mixture was extracted with CH₂Cl₂ (3×15 mL), and the combined organic layers were dried over anhydrous Na₂SO₄, filtered, and then concentrated under reduced pressure. The residue was purified by silica gel column chromatography to afford methyl (R)-2-((1-(3-isobutoxy-2,7-dimethyl-1-oxo-1,2-dihydroisoquinolin-5-yl)ethyl)amino)benzoate (100 mg, 47% yield) as a white solid. MS: (ES⁺) m/z=423.2 [M+H]⁺.

### Step 2: Preparation of (R)-2-((1-(3-isobutoxy-2,7-dimethyl-1-oxo-1,2-dihydroisoquinolin-5-yl)ethyl)amino)benzoic acid

To a stirred solution of methyl (R)-2-((1-(3-isobutoxy-2,7-dimethyl-1-oxo-1,2-dihydroisoquinolin-5-yl)ethyl)amino)benzoate (100 mg, 0.24 mmol) in MeOH (5 mL) and H₂O (1 mL) was added NaOH (95 mg, 2.4 mmol) at room temperature. The reaction mixture was stirred overnight at 50° C. The mixture was acidified to pH 3 with 1 N HCl (aqueous). The aqueous layer was extracted with EtOAc (3×10 mL), and the combined organic layers were dried over anhydrous Na₂SO₄ and then concentrated under reduced pressure. The crude product was purified by prep-HPLC to afford (R)-2-((1-(3-isobutoxy-2,7-dimethyl-1-oxo-1,2-dihydroisoquinolin-5-yl)ethyl)amino)benzoic acid (9.4 mg, 10% yield, >99% ee) as a white solid. ¹H NMR (400 MHz, DMSO-d₆) δ 7.86-7.77 (m, 2H), 7.40-7.35 (m, 1H), 7.13 (s, 1H), 6.50-6.45 (m, 1H), 6.32-6.28 (m, 1H), 6.17 (s, 1H), 5.14 (s, 1H), 3.95-3.88 (m, 2H), 3.45 (s, 3H), 2.30 (s, 3H), 2.15-2.05 (m, 1H), 1.51-1.45 (m, 3H), 1.05-1.00 (m, 6H). MS: (ES⁻) m/z=407.2 [M−H]⁻.

### Example 41: (R)-5-fluoro-2-((1-(4-fluoro-2,7-dimethyl-1-oxo-3-(4-(2,2,2-trifluoroethyl)piperazin-1-yl)-1,2-dihydroisoquinolin-5-yl)ethyl)amino)benzoic acid

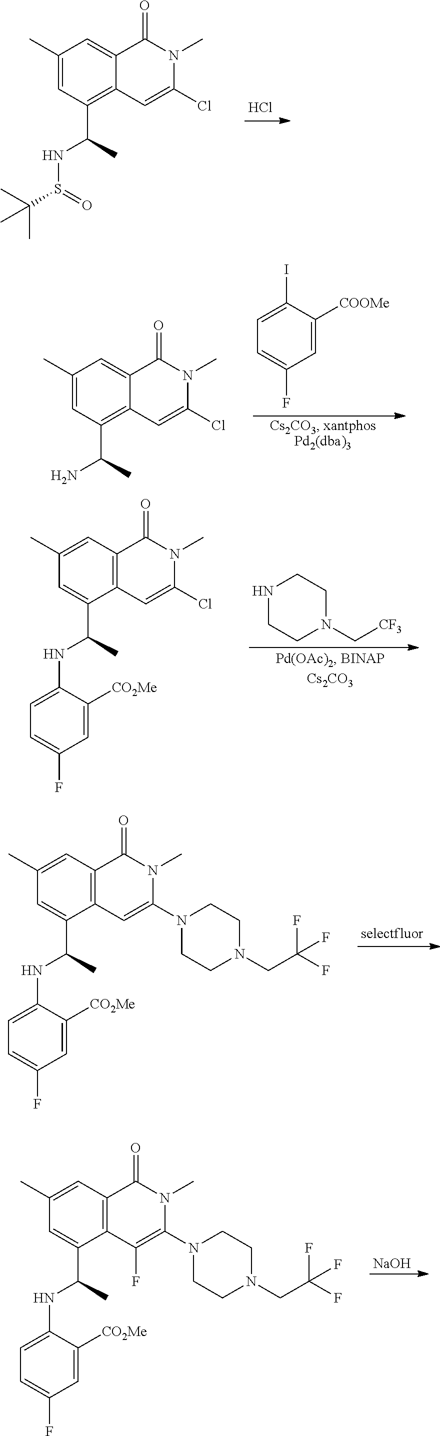

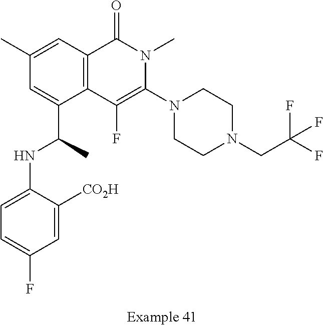 [SMILES: Cc1cc([C@@H](C)Nc2ccc(F)cc2C(=O)O)c2c(F)c(N3CCN(CC(F)(F)F)CC3)n(C)c(=O)c2c1]

### Step 1: Preparation of (R)-5-(1-aminoethyl)-3-chloro-2,7-dimethylisoquinolin-1 (2H)-one

To a stirred solution of (R)—N-[(1R)-1-(3-chloro-2,7-dimethyl-1-oxoisoquinolin-5-yl)ethyl]-2-methylpropane-2-sulfinamide (1 g, 2.8 mmol) in MeOH (10 mL) was added HCl in 1,4-dioxane (4 M, 10 mL) at room temperature. The resulting mixture was stirred for 1 h at room temperature. The resulting mixture was concentrated under reduced pressure. The crude product was used in the next step directly without further purification. MS: (ES⁺) m/z=250.9 [M+H]⁺.

### Step 2: Preparation of methyl (R)-2-((1-(3-chloro-2,7-dimethyl-1-oxo-1,2-dihydroisoquinolin-5-yl)ethyl)amino)-5-fluorobenzoate

A solution of (R)-5-(1-aminoethyl)-3-chloro-2,7-dimethylisoquinolin-1 (2H)-one (1.32 g, 4 mmol) in 1,4-dioxane (20 mL) was treated with methyl 5-fluoro-2-iodobenzoate (2.95 g, 10.5 mmol), Xantphos (0.61 g, 1.1 mmol), Cs₂CO₃ (6.86 g, 21 mmol) and Pd₂(dba)₃ (0.48 g, 0.52 mmol) at room temperature. The mixture was stirred overnight at 90° C. under a nitrogen atmosphere. The reaction was quenched with water (30 mL) at room temperature. The aqueous layer was extracted with EtOAc (3×150 mL), and the combined organic layers were dried over anhydrous Na₂SO₄ and concentrated under reduced pressure. The residue was purified by silica gel column chromatography to afford methyl (R)-2-((1-(3-chloro-2,7-dimethyl-1-oxo-1,2-dihydroisoquinolin-5-yl)ethyl)amino)-5-fluorobenzoate (530 mg, 25% yield) as a brown yellow solid. MS: (ES⁺) m/z=403.0 [M+H]⁺.

### Step 3: Preparation of methyl (R)-2-((1-(2,7-dimethyl-1-oxo-3-(4-(2,2,2-trifluoroethyl)piperazin-1-yl)-1,2-dihydroisoquinolin-5-yl)ethyl)amino)-5-fluorobenzoate

To a stirred mixture of methyl (R)-2-((1-(3-chloro-2,7-dimethyl-1-oxo-1,2-dihydroisoquinolin-5-yl)ethyl)amino)-5-fluorobenzoate (100 mg, 0.25 mmol) and 1-(2,2,2-trifluoroethyl)piperazine (104 mg, 0.62 mmol) in 1,4-dioxane (1 mL) were added Cs₂CO₃ (162 mg, 0.5 mmol), Pd(OAc)₂ (5.6 mg, 0.025 mmol), and BINAP (15.5 mg, 0.025 mmol) at room temperature. The resulting mixture was stirred overnight at 100° C. under a nitrogen atmosphere. The resulting mixture was concentrated under vacuum. The residue was purified by prep-TLC (PE:EA=1:1) to afford methyl (R)-2-((1-(2,7-dimethyl-1-oxo-3-(4-(2,2,2-trifluoroethyl)piperazin-1-yl)-1,2-dihydroisoquinolin-5-yl)ethyl)amino)-5-fluorobenzoate (90 mg, 68% yield) as a yellow solid. MS: (ES⁺) m/z=535.3 [M+H]⁺.

### Step 4: Preparation of methyl (R)-5-fluoro-2-((1-(4-fluoro-2,7-dimethyl-1-oxo-3-(4-(2,2,2-trifluoroethyl)piperazin-1-yl)-1,2-dihydroisoquinolin-5-yl)ethyl)amino)benzoate

To a stirred solution of methyl (R)-2-((1-(2,7-dimethyl-1-oxo-3-(4-(2,2,2-trifluoroethyl)piperazin-1-yl)-1,2-dihydroisoquinolin-5-yl)ethyl)amino)-5-fluorobenzoate (180 mg, 0.34 mmol) in acetonitrile (1 mL) were added Selectfluor™ (107 mg, 0.3 mmol) and acetic acid (4 mg, 0.07 mmol) at room temperature. The resulting mixture was stirred overnight at 60° C. The resulting mixture was concentrated under reduced pressure. The residue was purified by prep-TLC (PE:EA=1:1) to afford methyl (R)-5-fluoro-2-((1-(4-fluoro-2,7-dimethyl-1-oxo-3-(4-(2,2,2-trifluoroethyl)piperazin-1-yl)-1,2-dihydroisoquinolin-5-yl)ethyl)amino)benzoate (69 mg, 37% yield) as a yellow solid. MS: (ES⁺) m/z=553.2 [M+H]⁺.

### Step 5: Preparation of (R)-5-fluoro-2-((1-(4-fluoro-2,7-dimethyl-1-oxo-3-(4-(2,2,2-trifluoroethyl)piperazin-1-yl)-1,2-dihydroisoquinolin-5-yl)ethyl)amino)benzoic acid

To a stirred solution of methyl (R)-5-fluoro-2-((1-(4-fluoro-2,7-dimethyl-1-oxo-3-(4-(2,2,2-trifluoroethyl)piperazin-1-yl)-1,2-dihydroisoquinolin-5-yl)ethyl)amino)benzoate (69 mg, 0.13 mmol) in MeOH (5 ml) were added H₂O (2 mL) and NaOH (100 mg, 0.25 mmol) dropwise at room temperature. The resulting mixture was stirred for 30 min at 70° C. The reaction was diluted with water (10 mL) at room temperature. The aqueous layer was extracted with CH₂Cl₂ (3×20 mL). The organic layers were combined, dried with Na₂SO₄, filtered, and concentrated under reduced pressure. The crude product was purified by prep-HPLC to afford (R)-5-fluoro-2-((1-(4-fluoro-2,7-dimethyl-1-oxo-3-(4-(2,2,2-trifluoroethyl)piperazin-1-yl)-1,2-dihydroisoquinolin-5-yl)ethyl)amino)benzoic acid (20 mg, 30% yield, >99% ee) as a yellow solid. ¹H-NMR (400 MHz, Methanol-d₄) δ 8.07 (m, 1H), 7.67 (s, 1H), 7.59 (m, 1H), 6.94 (m, 1H), 6.28 (m, 1H), 5.37 (m, 1H), 3.65 (s, 3H), 3.54-3.40 (m, 2H), 3.23-3.03 (m, 4H), 3.05-2.95 (m, 2H), 2.76-2.55 (m, 2H), 2.36 (s, 3H), 1.65-1.55 (m, 3H). MS: (ES⁺) m/z=539.1 [M+H]⁺.

### Example 42: (R)-2-((1-(3-(3,5′-difluoro-1′-methyl-2′-oxo-1′,2′-dihydro-[2,4′-bipyridin]-5-yl)-7-fluoro-2-methyl-1-oxo-1,2-dihydroisoquinolin-5-yl)ethyl)amino)benzoic acid

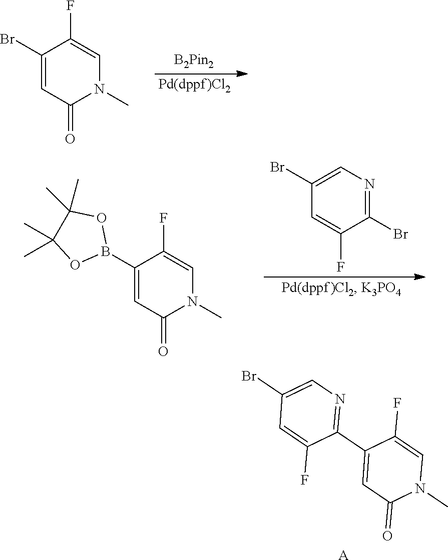

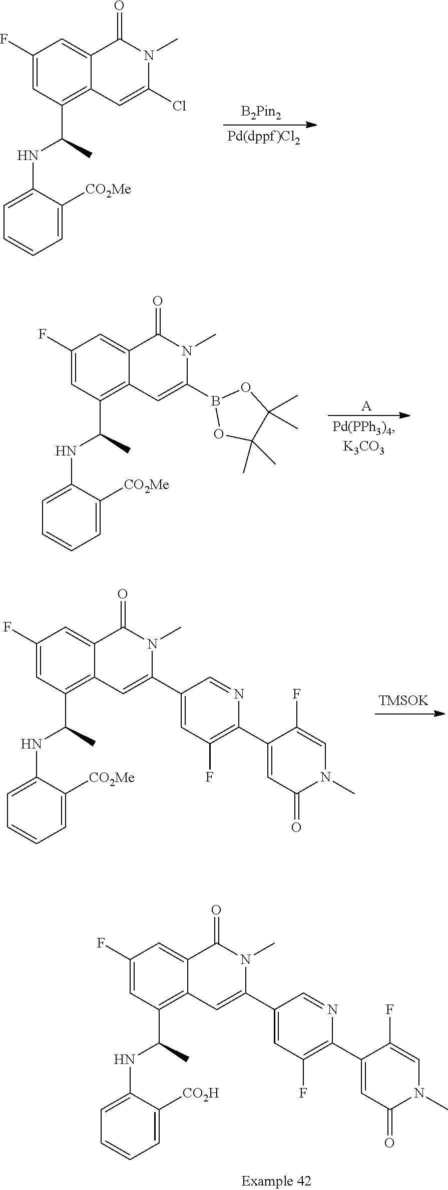

### Step 1: Preparation of 5-fluoro-1-methyl-4-(4,4,5,5-tetramethyl-1,3,2-dioxaborolan-2-yl)pyridin-2 (1H)-one

Into a 40-mL vial, a solution of 4-bromo-5-fluoro-1-methylpyridin-2-one (500 mg, 2.4 mmol), 4,4,5,5-tetramethyl-2-(4,4,5,5-tetramethyl-1,3,2-dioxaborolan-2-yl)-1,3,2-dioxaborolane (924 mg, 3.6 mmol) and KOAc (715 mg, 7.3 mmol), and Pd(dppf)Cl₂ (53 mg, 0.073 mmol) in dioxane (10 mL) was stirred for 1 h at 100° C. under a nitrogen atmosphere. The resulting mixture was filtered, and the filter cake was washed with EtOAc. The filtrate was concentrated under reduced pressure to afford 5-fluoro-1-methyl-4-(4,4,5,5-tetramethyl-1,3,2-dioxaborolan-2-yl)pyridin-2 (1H)-one (500 mg, 81% yield) as a black oil which was used in next step without further purification. MS: (ES⁺) m/z=254.1 [M+H]⁺.

### Step 2: Preparation of 5-bromo-3,5′-difluoro-1′-methyl-[2,4′-bipyridin]-2(1H)-one

Into a 40-mL vial, a solution of 5-fluoro-1-methyl-4-(4,4,5,5-tetramethyl-1,3,2-dioxaborolan-2-yl)pyridin-2 (1H)-one (500 mg, 2 mmol), 2,5-dibromo-3-fluoropyridine (2.52 g, 9.9 mmol), Pd(dppf)Cl₂ (289 mg, 0.4 mmol), and K₃PO₄ (1.26 g, 5.9 mmol) in dioxane (5 mL) and H₂O (1 mL) was stirred for 1 h at 100° C. under a nitrogen atmosphere. The resulting mixture was quenched with 20 mL H₂O and extracted with EtOAc (3×10 mL). The combined organic layers were washed with brine (3×10 mL) and dried over anhydrous Na₂SO₄. After filtration, the filtrate was concentrated under reduced pressure. The residue was purified by prep-TLC (CH₂Cl₂:MeOH=15:1) to afford 5-bromo-3,5′-difluoro-1′-methyl-[2,4′-bipyridin]-2′(1′H)-one (300 mg, 50% yield) as a yellow oil. MS: (ES⁺) m/z=301.0 [M+H]⁺.

### Step 3: Preparation of methyl (R)-2-((1-(7-fluoro-2-methyl-1-oxo-3-(4,4,5,5-tetramethyl-1,3,2-diox aborolan-2-yl)-1,2-dihydroisoquinolin-5-yl)ethyl)amino)benzoate

Into a 40 mL vial, a solution of methyl (R)-2-((1-(3-chloro-7-fluoro-2-methyl-1-oxo-1,2-dihydroisoquinolin-5-yl)ethyl)amino)benzoate (500 mg, 1.3 mmol), 4,4,5,5-tetramethyl-2-(4,4,5,5-tetramethyl-1,3,2-dioxaborolan-2-yl)-1,3,2-dioxaborolane (653 mg, 2.6 mmol), KOAc (379 mg, 3.9 mmol), and Pd(dppf)Cl₂ (94 mg, 0.13 mmol) in dioxane (10 mL) was stirred overnight at 100° C. under a nitrogen atmosphere. The resulting mixture was filtered, and the filter cake was washed with EtOAc (3×5 mL). The filtrate was concentrated under reduced pressure to afford methyl (R)-2-((1-(7-fluoro-2-methyl-1-oxo-3-(4,4,5,5-tetramethyl-1,3,2-diox aborolan-2-yl)-1,2-dihydroisoquinolin-5-yl)ethyl)amino)benzoate (500 mg, 81% yield) as a black oil. The crude product was used in the next step directly without further purification. MS: (ES⁺) m/z=481.2 [M+H]⁺.

### Step 4: Preparation of methyl (R)-2-((1-(3-(3,5′-difluoro-1′-methyl-2′-oxo-1′,2′-dihydro-[2,4′-bipyridin]-5-yl)-7-fluoro-2-methyl-1-oxo-1,2-dihydroisoquinolin-5-yl)ethyl)amino)benzoate

Into a 40 mL vial, a solution of methyl (R)-2-((1-(7-fluoro-2-methyl-1-oxo-3-(4,4,5,5-tetramethyl-1,3,2-diox aborolan-2-yl)-1,2-dihydroisoquinolin-5-yl)ethyl)amino)benzoate (500 mg, 1 mmol), 5-bromo-3,5′-difluoro-1′-methyl-[2,4′-bipyridin]-2′-one (376 mg, 1.25 mmol), Pd(PPh₃)₄ (241 mg, 0.21 mmol), and K₂CO₃ (288 mg, 2.1 mmol) in dioxane (6 mL) and H₂O (1 mL) was stirred for 3 h at 100° C. under a nitrogen atmosphere. The resulting mixture quenched with 20 mL H₂O and the mixture was extracted with EtOAc (3×20 mL). The combined organic layers were washed with brine (3×20 mL) and dried over anhydrous Na₂SO₄. After filtration, the filtrate was concentrated under reduced pressure. The residue was purified by prep-TLC (CH₂Cl₂:MeOH=20:1) to afford methyl (R)-2-((1-(3-(3,5′-difluoro-1′-methyl-2′-oxo-1′,2′-dihydro-[2,4′-bipyridin]-5-yl)-7-fluoro-2-methyl-1-oxo-1,2-dihydroisoquinolin-5-yl)ethyl)amino)benzoate (300 mg, 50% yield) as a yellow oil. MS: (ES⁺) m/z=575.2 [M+H]⁺.

### Step 5: Preparation of (R)-2-((1-(3-(3,5′-difluoro-1′-methyl-2′-oxo-1′,2′-dihydro-[2,4′-bipyridin]-5-yl)-7-fluoro-2-methyl-1-oxo-1,2-dihydroisoquinolin-5-yl)ethyl)amino)benzoic acid

Into a 20 mL vial were added methyl (R)-2-((1-(3-(3,5′-difluoro-1′-methyl-2′-oxo-1′,2′-dihydro-[2,4′-bipyridin]-5-yl)-7-fluoro-2-methyl-1-oxo-1,2-dihydroisoquinolin-5-yl)ethyl)amino)benzoate (300 mg, 0.52 mmol), potassium trimethylsilanolate (670 mg, 5.2 mmol) and THF (10 mL) at room temperature. The resulting mixture was stirred overnight at room temperature. The mixture was acidified to pH 5 by the addition of a saturated aqueous NaH₂PO₄ solution. The resulting mixture was extracted with EtOAc (3×10 mL). The combined organic layers were washed with brine (3×10 mL) and dried over anhydrous Na₂SO₄. After filtration, the filtrate was concentrated under reduced pressure. The crude product was purified by prep-TLC (DCM:MeOH=15:1) and then by prep-achiral-SFC to afford (R)-2-((1-(3-(3,5′-difluoro-1′-methyl-2′-oxo-1′,2′-dihydro-[2,4′-bipyridin]-5-yl)-7-fluoro-2-methyl-1-oxo-1,2-dihydroisoquinolin-5-yl)ethyl)amino)benzoic acid (74 mg, 25% yield, 97.9% ee) as an off-white solid. ¹H NMR (400 MHz, DMSO-d6) δ 12.82 (s, 1H), 8.90-8.85 (m, 1H), 8.40 (d, J=5.9 Hz, 1H), 8.35-8.30 (m, 1H), 8.20 (d, J=6.5 Hz, 1H), 7.90-7.85 (m, 1H), 7.85-7.82 (m, 1H), 7.55-7.48 (m, 1H), 7.33 (s, 1H), 7.19-7.08 (m, 1H), 6.80 (d, J=7.5 Hz, 1H), 6.58-6.50 (m, 1H), 6.29-6.20 (m, 1H), 5.34-5.27 (m, 1H), 3.48 (s, 3H), 3.40 (s, 3H), 1.54 (d, J=6.5 Hz, 3H); MS: (ES⁺) m/z=561.2 [M+H]⁺.

### Example 43: (R)-2-((1-(3-cyclopropyl-2,7-dimethyl-1-oxo-1,2-dihydroisoquinolin-5-yl)ethyl)amino)benzoic acid

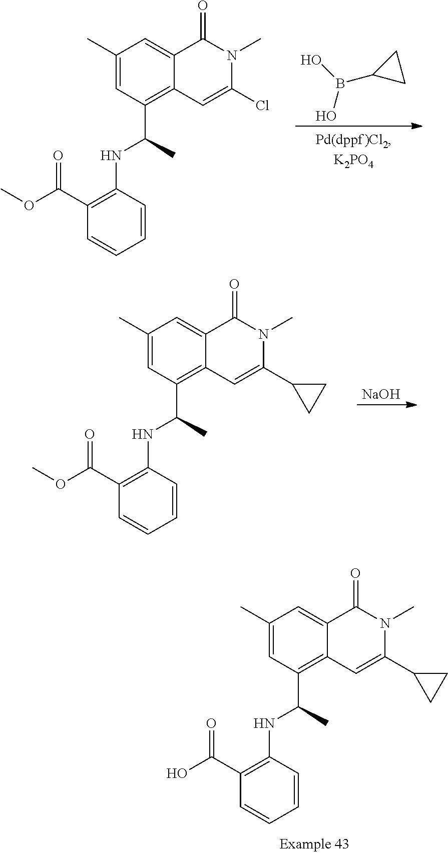

### Step 1: Preparation of methyl (R)-2-((1-(3-cyclopropyl-2,7-dimethyl-1-oxo-1,2-dihydroisoquinolin-5-yl)ethyl)amino)benzoate

Into a 250 mL round-bottle flask were added methyl (R)-2-((1-(3-chloro-2,7-dimethyl-1-oxo-1,2-dihydroisoquinolin-5-yl)ethyl)amino)benzoate (2.3 g, 1.3 mmol), cyclopropylboronic acid (1.0 g, 0.1 mmol), K₃PO₄ (3.7 g, 3.9 mmol), dioxane (25 mL), H₂O (25 mL) and Pd(dppf)Cl₂ (428 mg, 0.13 mmol) at room temperature. The resulting mixture was stirred overnight at 100° C. under a nitrogen atmosphere. The resulting mixture quenched with 20 mL H₂O and extracted with CH₂Cl₂ (3×50 mL). The combined organic layers were washed with brine (2×30 mL) and dried over anhydrous Na₂SO₄. After filtration, the filtrate was concentrated under reduced pressure. The residue was purified by silica gel column chromatography to afford methyl (R)-2-((1-(3-cyclopropyl-2,7-dimethyl-1-oxo-1,2-dihydroisoquinolin-5-yl)ethyl)amino)benzoate (1.1 g, 47% yield) as a yellow solid. MS: (ES⁺) m/z=391.3 [M+H]⁺.

### Step 2: Preparation of (R)-2-((1-(3-cyclopropyl-2,7-dimethyl-1-oxo-1,2-dihydroisoquinolin-5-yl)ethyl)amino)benzoic acid

Into a 20 mL vial were added methyl (R)-2-((1-(3-cyclopropyl-2,7-dimethyl-1-oxo-1,2-dihydroisoquinolin-5-yl)ethyl)amino)benzoate (150 mg, 0.38 mmol), MeOH (3 mL), H₂O (2 mL), THF (3 mL) and NaOH (154 mg, 3.8 mmol) at room temperature. The resulting mixture was stirred at 50° C. for 4 hr. The mixture was acidified to pH 4 with aqueous HCl (1N) and the resulting mixture was extracted with CH₂Cl₂ (3×50 mL). The combined organic layers were washed with brine (2×50 mL) and then dried over anhydrous Na₂SO₄. After filtration, the filtrate was concentrated under reduced pressure. The crude product was purified by prep-HPLC to afford (R)-2-((1-(3-cyclopropyl-2,7-dimethyl-1-oxo-1,2-dihydroisoquinolin-5-yl)ethyl)amino)benzoic acid (55 mg, 35% yield, >99% ee) as a white solid. ¹H-NMR (400 MHz, DMSO-d6) δ 8.54 (s, 1H), 7.92 (s, 1H), 7.80 (d, J=6.8 Hz, 1H), 7.45 (d, J=1.9 Hz, 1H), 7.15-7.13 (m, 1H), 6.65 (s, 1H), 6.52-6.50 (m, 1H), 6.33 (d, J=8.4 Hz, 1H), 5.18-5.10 (m, 1H), 3.69 (s, 3H), 2.34 (s, 3H), 2.06-2.04 (m, 1H), 1.51 (d, J=6.5 Hz, 3H), 1.06-0.94 (m, 2H), 0.84-0.83 (m, 2H). MS (ES⁻) m/z=375.1 [M−H].

### Example 44: 2-(((1R)-1-(3-(3-ethoxy-3,8-diazabicyclo[3.2.1]octan-8-yl)-7-fluoro-2-methyl-1-oxo-1,2-dihydroisoquinolin-5-yl)ethyl)amino)benzoic acid

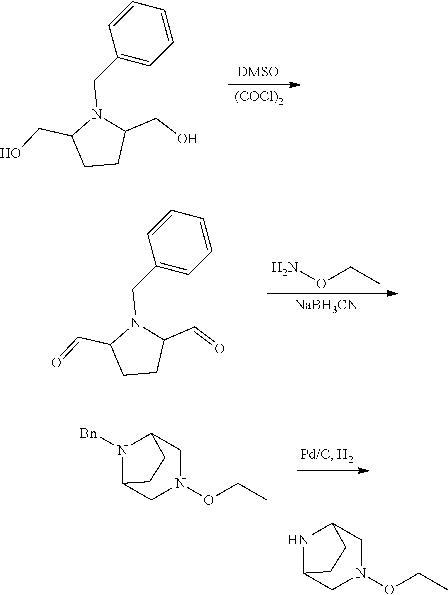

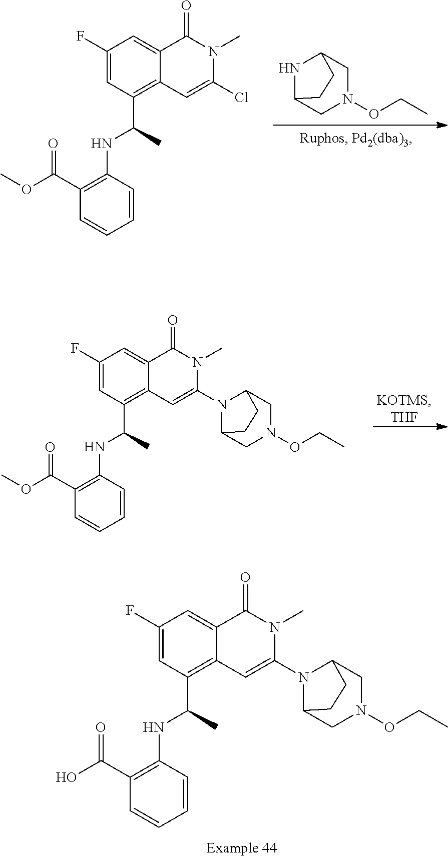

### Step 1: Preparation of 1-benzylpyrrolidine-2,5-dicarbaldehyde

To a stirred solution of oxalyl chloride (3.44 g, 27.1 mmol) in DCM (50 mL) was added DMSO (4.24 g, 54.2 mmol) dropwise at −78° C. under a nitrogen atmosphere. The resulting mixture was stirred for 15 minutes at −78° C. To the above mixture was added [1-benzyl-5-(hydroxymethyl) pyrrolidin-2-yl]methanol (1 g, 4.5 mmol) in DCM (5 mL) dropwise at −78° C. The resulting mixture was stirred for 30 minutes. To the above mixture was added Et₃N (5.49 g, 54.2 mmol) in DCM (5 mL) dropwise at −78° C. The resulting mixture was stirred for 1 hr at room temperature. The resulting mixture was quenched with saturated aqueous sodium bicarbonate (100 mL) and extracted with EtOAc (2×100 mL). The combined organic layers were washed with brine (2×100 mL) and dried over anhydrous Na₂SO₄. After filtration, the filtrate was concentrated under reduced pressure to afford the crude product (1-benzylpyrrolidine-2,5-dicarbaldehyde (980 mg) which was used in the next step directly without further purification. MS (ES⁺) m/z=218.1 [M+H]⁺.

### Step 2: Preparation of 8-benzyl-3-ethoxy-3,8-diazabicyclo[3.2.1]octane

To a stirred solution of 1-benzylpyrrolidine-2,5-dicarbaldehyde (1.46 g, 6.7 mmol) and O-ethylhydroxylamine hydrochloride (1.31 g, 13.4 mmol) in MeOH (40 mL) were added HOAc (2.42 g, 40.3 mmol) and NaBH₃CN (2.53 g, 40.3 mmol) at 0° C. The resulting mixture was stirred for 12 h at room temperature and then the reaction was quenched with H₂O (100 mL). The resulting mixture was extracted with EtOAc (3×50 mL). The combined organic layers were washed with brine (2×50 mL) and dried over anhydrous Na₂SO₄. After filtration, the filtrate was concentrated under reduced pressure. The residue was purified by reversed-phase flash chromatography to afford 8-benzyl-3-ethoxy-3,8-diazabicyclo[3.2.1]octane (750 mg, 45% yield) as a yellow oil. MS: (ES⁺) m/z=247.2 [M+H]⁺.

### Step 3: Preparation of 3-ethoxy-3,8-diazabicyclo[3.2.1]octane

To a stirred solution of 8-benzyl-3-ethoxy-3,8-diazabicyclo[3.2.1]octane (1 g, 4.1 mmol) in MeOH (100 mL) was added Pd/C (2.0 g, 18.8 mmol) in a pressure tank reactor. The mixture was hydrogenated at room temperature under 50 atm of hydrogen pressure for 12 h. The reaction mixture was filtered through a Celite pad and concentrated under reduced pressure. The crude product was purified by Prep-HPLC to afford 3-ethoxy-3,8-diazabicyclo[3.2.1]octane (216 mg, 34% yield) as a yellow oil. MS: (ES⁺) m/z=157.1 [M+H]⁺.

### Step 4: Preparation of methyl 2-(((1R)-1-(3-(3-ethoxy-3,8-diazabicyclo[3.2.1]octan-8-yl)-7-fluoro-2-methyl-1-oxo-1,2-dihydroisoquinolin-5-yl)ethyl)amino)benzoate

To a stirred solution of methyl (R)-2-((1-(3-chloro-7-fluoro-2-methyl-1-oxo-1,2-dihydroisoquinolin-5-yl)ethyl)amino)benzoate (180 mg, 0.46 mmol) and 3-ethoxy-3,8-diazabicyclo[3.2.1]octane (216 mg, 1.4 mmol) in dioxane (5 mL) were added Pd₂(dba)₃ (42 mg, 0.046 mmol), Ruphos (43 mg, 0.092 mmol), and Cs₂CO₃ (451 mg, 1.4 mmol). The resulting mixture was stirred for 12 h at 100° C. under a nitrogen atmosphere. The resulting mixture was diluted with H₂O (20 mL). The resulting mixture was extracted with EtOAc (3×20 mL). The combined organic layers were washed with brine (1×50 mL) and dried over anhydrous Na₂SO₄. After filtration, the filtrate was concentrated under reduced pressure. The residue was purified by silica gel column chromatography to afford methyl 2-(((1R)-1-(3-(3-ethoxy-3,8-diazabicyclo[3.2.1]octan-8-yl)-7-fluoro-2-methyl-1-oxo-1,2-dihydroisoquinolin-5-yl)ethyl)amino)benzoate (170 mg, 73% yield) as a yellow solid. MS: (ES⁺) m/z=509.3 [M+H]⁺.

### Step 5: Preparation of 2-(((1R)-1-(3-(3-ethoxy-3,8-diazabicyclo[3.2.1]octan-8-yl)-7-fluoro-2-methyl-1-oxo-1,2-dihydroisoquinolin-5-yl)ethyl)amino)benzoic acid

To a stirred solution of methyl 2-(((1R)-1-(3-(3-ethoxy-3,8-diazabicyclo[3.2.1]octan-8-yl)-7-fluoro-2-methyl-1-oxo-1,2-dihydroisoquinolin-5-yl)ethyl)amino)benzoate (170 mg, 0.33 mmol) in THF (10 mL) was added trimethyl(potassiooxy) silane (429 mg, 3.3 mmol). The resulting mixture was stirred for 2 h at room temperature. The mixture was acidified to pH 6 with aqueous HCl (1 N). The resulting mixture was diluted with H₂O (20 mL) and extracted with EtOAc (3×30 mL). The combined organic layers were washed with brine (2×50 mL) and then dried over anhydrous Na₂SO₄. After filtration, the filtrate was concentrated under reduced pressure. The crude product was purified by Prep-HPLC to afford 2-(((1R)-1-(3-(3-ethoxy-3,8-diazabicyclo[3.2.1]octan-8-yl)-7-fluoro-2-methyl-1-oxo-1,2-dihydroisoquinolin-5-yl)ethyl)amino)benzoic acid (95 mg, 57% yield, >99% ee) as an off-white solid. ¹H NMR (400 MHz, Methanol-d4) δ 7.97-7.90 (m, 1H), 7.80-7.73 (m, 1H), 7.48-7.40 (m, 1H), 7.24-7.16 (m, 1H), 6.63-6.54 (m, 1H), 6.41 (d, J=8.4 Hz, 1H), 6.33 (s, 1H), 5.14-5.04 (m, 1H), 3.88 (m, 2H), 3.80-3.73 (m, 2H), 3.71 (s, 3H), 3.32-3.25 (m, 2H), 2.92-2.83 (m, 2H), 2.11-1.84 (m, 4H), 1.65 (d, J=6.7 Hz, 3H), 1.23-1.16 (m, 3H). MS (ES⁻) m/z=493.2 [M−H].

### Example 45: 3-(((R)-1-(3-(trans-[1,1′-bi(cyclopropan)]-2-yl)-7-fluoro-2-methyl-1-oxo-1,2-dihydroisoquinolin-5-yl)ethyl)amino)-6-methylpicolinic acid, diastereomer 1

### Example 46: -(((R)-1-(3-(trans-[1,1′-bi(cyclopropan)]-2-yl)-7-fluoro-2-methyl-1-oxo-1,2-dihydroisoquinolin-5-yl)ethyl)amino)-6-methylpicolinic acid, diastereomer 2

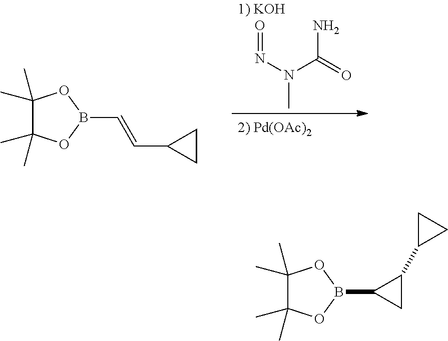

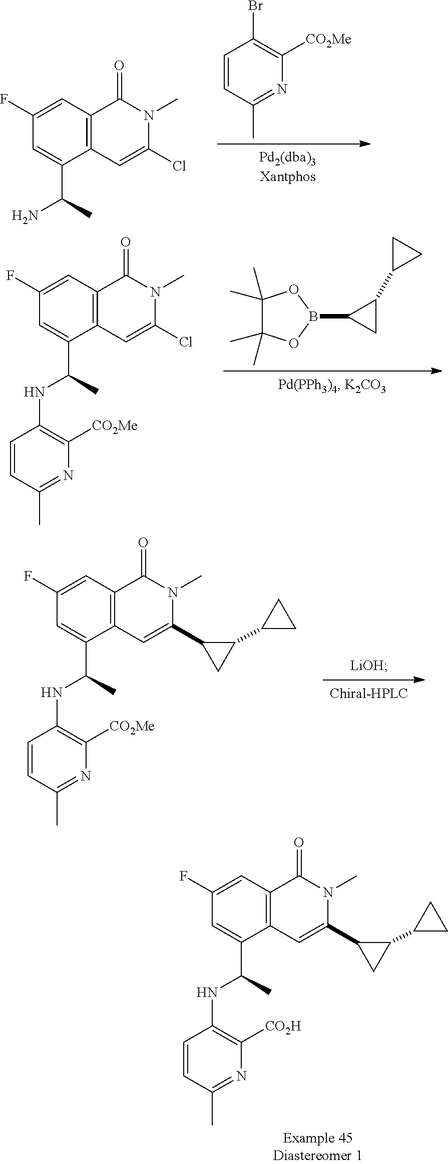

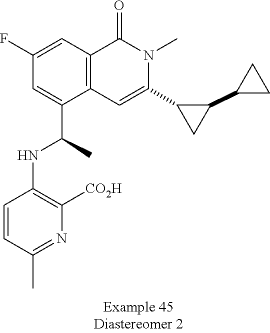

### Step 1: Preparation of 2-{[1,1′-bi(cyclopropane)]-2-yl}-4,4,5,5-tetramethyl-1,3,2-dioxaborolane

To a stirred solution of 1-methyl-1-nitrosourea (2.55 g, 24.7 mmol) in diethyl ether (20 mL) was added 20% potassium hydroxide aqueous solution (20 mL) at 0° C., and the mixture was stirred at this temperature for 1 h. Under ice bath conditions, the organic phase of the above mixed solution was added to a solution of 2-[(E)-2-cyclopropylethenyl]-4,4,5,5-tetramethyl-1,3,2-dioxaborolane (1 g, 5.2 mmol) in diethyl ether (20 mL), and then Pd(OAc)₂ (12 mg, 0.052 mmol) was added to the mixture. The mixture was then stirred for 30 minutes at 0° C. The resulting mixture was then filtered and washed with DCM (3×20 mL). The filtrate was concentrated under reduced pressure. The residue was purified by silica gel column chromatography to afford 2-{[1,1′-bi(cyclopropane)]-2-yl}-4,4,5,5-tetramethyl-1,3,2-dioxaborolane (500 mg, 47% yield) as a colorless oil. GCMS: 208.1.

### Step 2: Preparation of methyl (R)-3-((1-(3-chloro-7-fluoro-2-methyl-1-oxo-1,2-dihydroisoquinolin-5-yl)ethyl)amino)-6-methylpicolinate

To a stirred mixture of 5-isopropyl-2,3,7-trimethyl-1-methylidene-2H-naphthalene (400 mg, 1.8 mmol), methyl 3-bromo-6-methylpyridine-2-carboxylate (1.22 g, 5.3 mmol), Xantphos (205 mg, 0.35 mmol), and Cs₂CO₃ (1.73 g, 5.3 mmol) in toluene (20 mL) was added Pd₂(dba)₃ (162 mg, 0.18 mmol) at room temperature. The resulting mixture was stirred overnight at 110° C. under a nitrogen atmosphere. The resulting mixture was filtered, and the filter cake was washed with EtOAc (3×30 mL). The filtrate was concentrated under reduced pressure. The residue was purified by Prep-TLC to afford methyl (R)-3-((1-(3-chloro-7-fluoro-2-methyl-1-oxo-1,2-dihydroisoquinolin-5-yl)ethyl)amino)-6-methylpicolinate (270 mg, 38% yield) as a yellow solid. MS: (ES⁺) m/z=404.2 [M+H]⁺.

### Step 3: Preparation of methyl 3-(((R)-1-(3-(trans-[1,1′-bi(cyclopropan)]-2-yl)-7-fluoro-2-methyl-1-oxo-1,2-dihydroisoquinolin-5-yl)ethyl)amino)-6-methylpicolinate

To a stirred mixture of methyl (R)-3-((1-(3-chloro-7-fluoro-2-methyl-1-oxo-1,2-dihydroisoquinolin-5-yl)ethyl)amino)-6-methylpicolinate (240 mg, 0.59 mmol) and 2-{[1,1′-bi(cyclopropane)]-2-yl}-4,4,5,5-tetramethyl-1,3,2-dioxaborolane (619 mg, 2.97 mmol), and K₂CO₃ (246 mg, 1.78 mmol), was added Pd(PPh₃)₄ (69 mg, 0.059 mmol) in dioxane (10 mL) and H₂O (2 mL) at room temperature. The resulting mixture was stirred overnight at 100° C. The resulting mixture was quenched with H₂O (20 mL) and extracted with EtOAc (3×30 mL). The combined organic layers were washed with brine (1×30 mL) and dried over anhydrous Na₂SO₄. After filtration, the filtrate was concentrated under reduced pressure. The residue was purified by Prep-TLC to afford methyl 3-(((R)-1-(3-(trans-[1,1′-bi(cyclopropan)]-2-yl)-7-fluoro-2-methyl-1-oxo-1,2-dihydroisoquinolin-5-yl)ethyl)amino)-6-methylpicolinate (170 mg, 64% yield) as a yellow solid. MS: (ES⁺) m/z=450.2 [M+H]⁺.

### Step 4: Preparation of 3-(((R)-1-(3-(trans-[1,1′-bi(cyclopropan)]-2-yl)-7-fluoro-2-methyl-1-oxo-1,2-dihydroisoquinolin-5-yl)ethyl)amino)-6-methylpicolinic acid

To a stirred solution of methyl 3-(((R)-1-(3-(trans-[1,1′-bi(cyclopropan)]-2-yl)-7-fluoro-2-methyl-1-oxo-1,2-dihydroisoquinolin-5-yl)ethyl)amino)-6-methylpicolinate (170 mg, 0.38 mmol) in MeOH (5 mL) and H₂O (1 mL) was added LiOH. H₂O (79 mg, 1.9 mmol) at room temperature. The resulting mixture was stirred for 2 h at room temperature. The mixture was neutralized to pH 6 with aqueous 1M HCl. The resulting mixture was extracted with EtOAc (3×30 mL). The combined organic layers were washed with brine (1×30 mL) and dried over anhydrous Na₂SO₄. After filtration, the filtrate was concentrated under reduced pressure. The residue was purified by Prep-HPLC to afford 3-(((R)-1-(3-(trans-[1,1′-bi(cyclopropan)]-2-yl)-7-fluoro-2-methyl-1-oxo-1,2-dihydroisoquinolin-5-yl)ethyl)amino)-6-methylpicolinic acid (88 mg, 53% yield) as a yellow oil. MS: (ES⁺) m/z=436.4 [M+H]⁺.

The diastereomers of 3-(((R)-1-(3-(trans-[1,1′-bi(cyclopropan)]-2-yl)-7-fluoro-2-methyl-1-oxo-1,2-dihydroisoquinolin-5-yl)ethyl)amino)-6-methylpicolinic acid (80 mg) were separated by chiral HPLC to afford 3-(((R)-1-(3-(trans-[1,1′-bi(cyclopropan)]-2-yl)-7-fluoro-2-methyl-1-oxo-1,2-dihydroisoquinolin-5-yl)ethyl)amino)-6-methylpicolinic acid, diastereomer 1 (23 mg, 29% yield, >99% ee) as a white solid, ¹H NMR (400 MHz, Methanol-d4) δ 7.82-7.81 (m, 1H), 7.45 (d, J=9.2 Hz, 1H), 7.25-6.86 (m, 2H), 6.74 (s, 1H), 5.23-5.22 (m, 1H), 3.82 (s, 3H), 2.48-2.27 (m, 3H), 1.88-1.87 (m, 1H), 1.64 (d, J=6.5 Hz, 3H), 1.35-1.27 (m, 1H), 1.07-0.89 (m, 3H), 0.57-0.45 (m, 2H), 0.29-0.20 (m, 2H), MS: (ES⁺) m/z=436.4 [M+H]⁺; and 3-(((R)-1-(3-(trans-[1,1′-bi(cyclopropan)]-2-yl)-7-fluoro-2-methyl-1-oxo-1,2-dihydroisoquinolin-5-yl)ethyl)amino)-6-methylpicolinic acid, diastereomer 2 (22 mg, 27% yield, >98% ee) as a white solid, ¹H NMR (400 MHz, Methanol-d4) δ 7.83-7.82 (m, 1H), 7.45 (d, J=8.7 Hz, 1H), 7.30-6.92 (m, 2H), 6.73 (s, 1H), 5.25-5.24 (m, 1H), 3.83 (s, 3H), 2.55-2.22 (m, 3H), 1.89-1.87 (m, 1H), 1.66 (d, J=6.5 Hz, 3H), 1.33-1.30 (m, 1H), 1.11-0.91 (m, 3H), 0.57-0.47 (m, 2H), 0.29-0.21 (m, 2H). MS: (ES⁺) m/z=436.4 [M+H]⁺.

### Example 47: (R)-2-((1-(3-(2-([1,1′-bi(cyclopropan)]-1-yl)pyrimidin-5-yl)-7-fluoro-2-methyl-1-oxo-1,2-dihydroisoquinolin-5-yl)ethyl)amino)benzoic acid

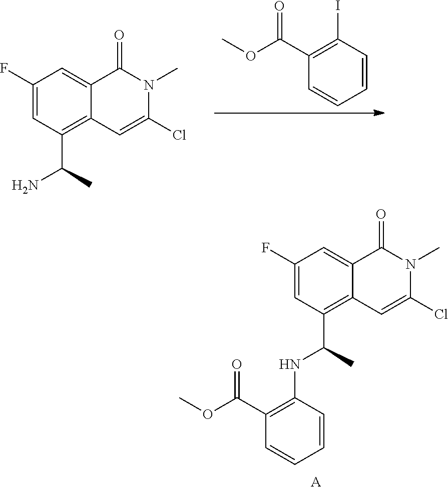

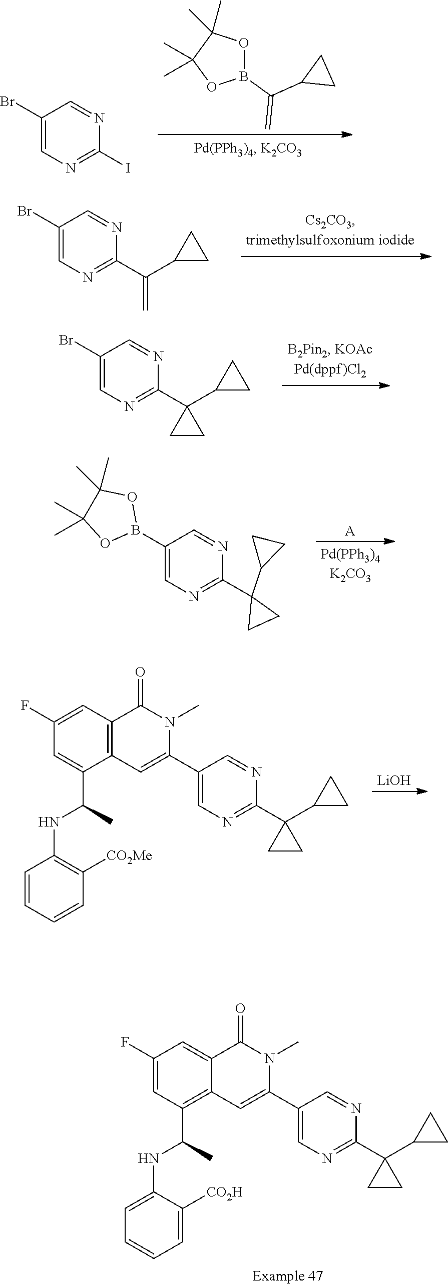

### Step 1: Preparation of methyl (R)-2-((1-(3-chloro-7-fluoro-2-methyl-1-oxo-1,2-dihydroisoquinolin-5-yl)ethyl)amino)benzoate

A mixture of (R)-5-(1-aminoethyl)-3-chloro-7-fluoro-2-methylisoquinolin-1 (2H)-one (2.00 g, 7.85 mmol), methyl 2-iodobenzoate (2.47 g, 9.4 mmol), Pd₂(dba)₃ (1.44 g, 1.6 mmol), Xantphos (909 mg, 1.6 mmol) and Cs₂CO₃ (6.4 g, 19.6 mmol) in dioxane (50 mL) was degassed and purged with nitrogen three times. The mixture was then stirred at 90° C. for 12 hr under a nitrogen atmosphere. The solution was filtered through a pad of celite and the filtrate was concentrated. The residue was purified by column chromatography (SiO₂, Petroleum ether/Ethyl acetate=100/1 to 30/1) to afford methyl (R)-2-((1-(3-chloro-7-fluoro-2-methyl-1-oxo-1,2-dihydroisoquinolin-5-yl)ethyl)amino)benzoate (1.2 g, 39% yield) as a white solid. LCMS (ESI) m/z=389.1 (M+H).

### Step 2: Preparation of 5-bromo-2-(1-cyclopropylvinyl)pyrimidine

A mixture of 5-bromo-2-iodo-pyrimidine (2.10 g, 7.4 mmol), 2-(1-cyclopropylvinyl)-4,4,5,5-tetramethyl-1,3,2-dioxaborolane (1.3 g, 6.7 mmol), Pd(dppf)Cl₂·CH₂Cl₂ (547 mg, 0.67 mmol), and Na₂CO₃ (2 g, 18.9 mmol) in dioxane (15 mL) and H₂O (1.5 mL) was degassed and purged with nitrogen three times. The mixture was then stirred at 90° C. for 24 hr under a nitrogen atmosphere. The reaction mixture was partitioned between H₂O (100 mL) and EtOAc (100 mL). The organic phase was separated, washed with brine (30 mL), dried over Na₂SO₄, filtered, and then concentrated under reduced pressure to give a residue. The residue was purified by prep-TLC (SiO₂, PE/EA=20/1) to afford 5-bromo-2-(1-cyclopropylvinyl)pyrimidine (440 mg, 17% yield) as colorless liquid. LCMS (ESI) m/z=225.1 (M+H); ¹H NMR (400 MHz, CDCl₃): δ 8.77 (s, 2H), 6.35 (s, 1H), 5.31 (s, 1H), 2.19-2.08 (m, 1H), 0.94-0.86 (m, 2H), 0.60-0.58 (m, 2H).

### Step 3: Preparation of 2-([1,1′-bi(cyclopropan)]-1-yl)-5-bromopyrimidine

To a stirred solution of 5-bromo-2-(1-cyclopropylvinyl)pyrimidine (440 mg, 1.95 mmol) and t-BuOK (329 mg, 2.9 mmol) in THF (10 mL) was added trimethylsulfoxonium iodide (645 mg, 2.93 mmol) in portions at 25° C. under a nitrogen atmosphere. The resulting mixture was stirred for 12 hours at 25° C. under nitrogen atmosphere. The reaction mixture was filtered, and the filtrate was concentrated under vacuum to afford a residue. The residue was purified by prep-TLC (SiO₂, PE/EA=20/1) to afford 2-([1,1′-bi(cyclopropan)]-1-yl)-5-bromopyrimidine (150 mg, 19% yield) as a colorless liquid. LCMS (ESI) m/z=238.9 (M+H).

### Step 4: Preparation of 2-([1,1′-bi(cyclopropan)]-1-yl)-5-(4,4,5,5-tetramethyl-1,3,2-dioxaborolan-2-yl)pyrimidine

A mixture of 2-([1,1′-bi(cyclopropan)]-1-yl)-5-bromopyrimidine (150 mg, 627 μmol), 4,4,5,5-tetramethyl-2-(4,4,5,5-tetramethyl-1,3,2-dioxaborolan-2-yl)-1,3,2-dioxaborolane (319 mg, 1.25 mmol), Pd(dppf)Cl₂·CH₂Cl₂ (51 mg, 63 μmol), and KOAc (185 mg, 1.88 mmol) in dioxane (4 mL) was degassed and purged with nitrogen three times. The mixture was then stirred at 90° C. for 12 hr under a nitrogen atmosphere. The reaction mixture was filtered and concentrated under reduced pressure to afford a residue. The residue was purified by prep-TLC (Petroleum ether/Ethyl acetate=20/1) to afford 2-([1,1′-bi(cyclopropan)]-1-yl)-5-(4,4,5,5-tetramethyl-1,3,2-dioxaborolan-2-yl)pyrimidine (179 mg, 99% yield) as a gray solid. ¹H NMR (400 MHz, CDCl₃): δ 8.90 (s, 2H), 1.92-1.84 (m, 1H), 1.35 (s, 12H), 1.25 (s, 2H), 0.83-0.75 (m, 2H), 0.60-0.53 (m, 2H), 0.09-0.05 (m, 2H).

### Step 5: Preparation of methyl (R)-2-((1-(3-(2-([1,1′-bi(cyclopropan)]-1-yl)pyrimidin-5-yl)-7-fluoro-2-methyl-1-oxo-1,2-dihydroisoquinolin-5-yl)ethyl)amino)benzoate

A mixture of 2-([1,1′-bi(cyclopropan)]-1-yl)-5-(4,4,5,5-tetramethyl-1,3,2-dioxaborolan-2-yl)pyrimidine (170 mg, 594 μmol), methyl (R)-2-((1-(3-chloro-7-fluoro-2-methyl-1-oxo-1,2-dihydroisoquinolin-5-yl)ethyl)amino)benzoate (150 mg, 386 μmol), Pd(dppf)Cl₂·CH₂Cl₂ (32 mg, 39 μmol) and Na₂CO₃ (123 mg, 1.2 mmol) in dioxane (3 mL) and H₂O (0.3 mL) was degassed and purged with nitrogen three times. The mixture was then stirred at 90° C. for 4 hr under a nitrogen atmosphere. The reaction mixture was then partitioned between H₂O (10 mL) and EtOAc (10 mL). The organic phase was separated, dried over Na₂SO₄, filtered, and concentrated under reduced pressure to afford a residue. The residue was purified by column chromatography (SiO₂, Petroleum ether/Ethyl acetate=100/1 to 10/1) to afford methyl (R)-2-((1-(3-(2-([1,1′-bi(cyclopropan)]-1-yl)pyrimidin-5-yl)-7-fluoro-2-methyl-1-oxo-1,2-dihydroisoquinolin-5-yl)ethyl)amino)benzoate (210 mg, 87% yield) as yellow oil. LCMS (ESI) m/z=513.3 (M+H);

¹H NMR (400 MHz, CDCl₃): δ 8.77 (s, 2H), 8.30 (d, J=5.2 Hz, 1H), 8.03-7.98 (m, 1H), 7.94 (d, J=8.4 Hz, 1H), 7.50-7.45 (m, 1H), 7.19-7.11 (m, 1H), 6.69 (s, 1H), 6.60-6.55 (m, 1H), 6.16 (d, J=8.4 Hz, 1H), 4.98-4.95 (m, 1H), 3.93 (s, 3H), 3.52 (s, 3H), 1.96-1.92 (m, 1H), 1.64 (d, J=6.8 Hz, 3H), 1.36-1.32 (m, 2H), 0.93-0.86 (m, 2H), 0.66-0.58 (m, 2H), 0.19-0.11 (m, 2H).

### Step 6: Preparation of (R)-2-((1-(3-(2-([1,1′-bi(cyclopropan)]-1-yl)pyrimidin-5-yl)-7-fluoro-2-methyl-1-oxo-1,2-dihydroisoquinolin-5-yl)ethyl)amino)benzoic acid

To a solution of methyl (R)-2-((1-(3-(2-([1,1′-bi(cyclopropan)]-1-yl)pyrimidin-5-yl)-7-fluoro-2-methyl-1-oxo-1,2-dihydroisoquinolin-5-yl)ethyl)amino)benzoate (200 mg, 390 μmol) in THF (1 mL) and MeOH (1 mL) was added aqueous LiOH. H₂O (2 M, 1 mL). The mixture was then stirred at 60° C. for 1 hr. The reaction mixture was adjusted pH to 5-6 by addition of aqueous HCl (1 N) at 25° C., and then diluted with H₂O (5 mL). The mixture was then extracted with EtOAc (3×5 mL). The combined organic layers were dried over Na₂SO₄, filtered, and concentrated under reduced pressure to afford a residue. The residue was purified by prep-HPLC to afford (R)-2-((1-(3-(2-([1,1′-bi(cyclopropan)]-1-yl)pyrimidin-5-yl)-7-fluoro-2-methyl-1-oxo-1,2-dihydroisoquinolin-5-yl)ethyl)amino)benzoic acid (99 mg, 51% yield) as a yellow solid. LCMS (ESI) m/z=499.1 (M+H); ¹H NMR (400 MHz, MeOD-d₄): δ 8.87 (s, 2H), 8.01-7.83 (m, 2H), 7.50-7.45 (m, 1H), 7.26-7.09 (m, 1H), 7.06 (s, 1H), 6.56 (t, J=8.0 Hz, 1H), 6.30 (d, J=8.0 Hz, 1H), 5.26-5.20 (m, 1H), 3.50 (s, 3H), 1.92-1.87 (m, 1H), 1.63 (d, J=6.8 Hz, 3H), 1.40-1.25 (m, 2H), 0.94-0.78 (m, 2H), 0.65-0.54 (m, 2H), 0.23-0.09 (m, 2H).

### Example 48: (R)-2-((1-(3-(6-((1-(difluoromethyl)-1H-pyrazol-4-yl)oxy)-5-fluoropyridin-3-yl)-7-fluoro-2-methyl-1-oxo-1,2-dihydroisoquinolin-5-yl)ethyl)amino)benzoic acid

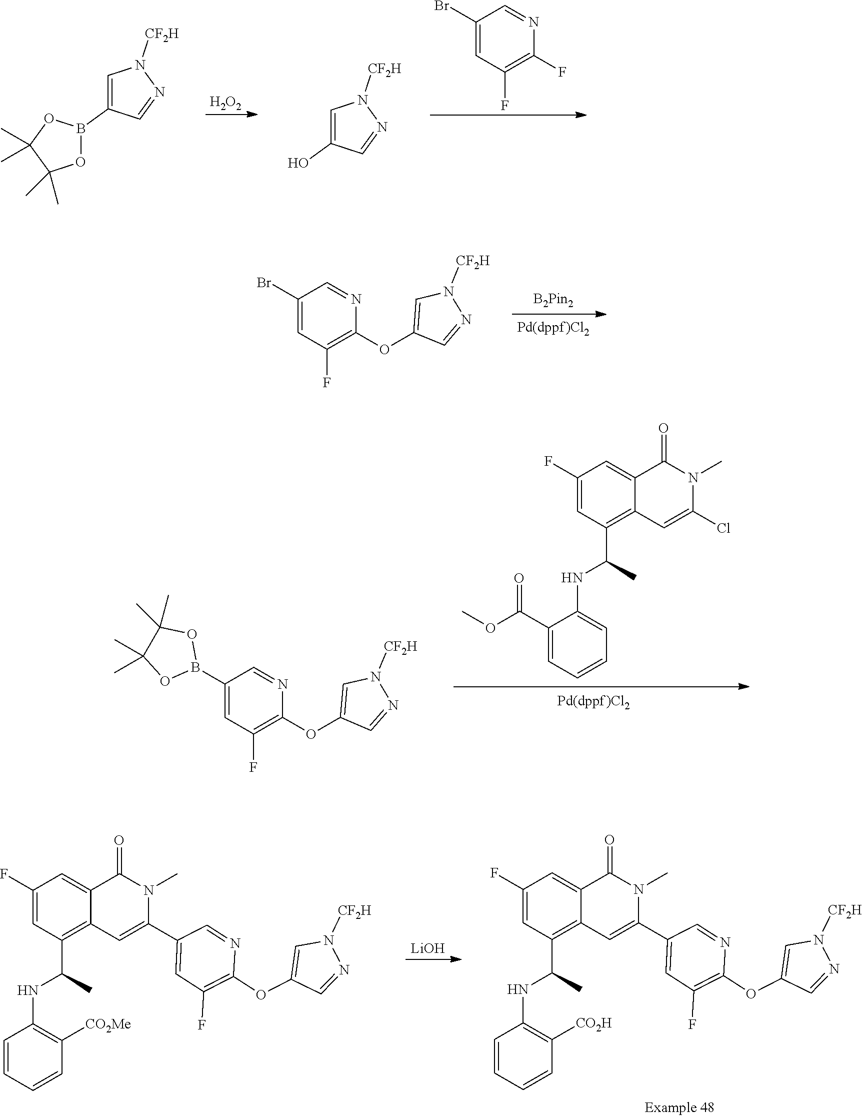

### Step 1: Preparation of 1-(difluoromethyl)-1H-pyrazol-4-ol

To a solution of 1-(difluoromethyl)-4-(4,4,5,5-tetramethyl-1,3,2-dioxaborolan-2-yl) pyrazole (2 g, 8.20 mmol) in THF (30 mL) was added H₂O₂ (30% purity, 1.86 g, 16.4 mmol) and NaOH (2 M, 8.2 mL). The mixture was stirred at 0° C. for 30 min. The reaction was quenched with water (50 mL), and the pH of the mixture was adjusted to 5-6 by adding aqueous hydrochloric acid (1 N). The mixture was extracted with EA (3×60 mL). The combined organic layers were washed sequentially with saturated sodium sulfite solution (200 mL) and brine (200 mL), dried over Na₂SO₄, filtered and concentrated under reduced pressure. The residue was purified by column to afford 1-(difluoromethyl) pyrazol-4-ol (900 mg, 82% yield) as yellow solid. ¹H NMR (400 MHz, CDCl₃): δ 7.43 (s, 1H), 7.40 (s, 1H), 7.04 (t, J=60.4 Hz, 1H).

### Step 2: Preparation of 5-bromo-2-((1-(difluoromethyl)-1H-pyrazol-4-yl)oxy)-3-fluoropyridine

To a solution of 5-bromo-2,3-difluoro-pyridine (1.29 g, 6.67 mmol) and 1-(difluoromethyl) pyrazol-4-ol (895 mg, 6.67 mmol) in DMF (15 mL) was added K₂CO₃ (2.77 g, 20.0 mmol). The mixture was stirred at 60° C. for 1 hr. The reaction was quenched with water (20 mL) and extracted with EA (3×15 mL). The combined organic layers were washed with brine (60 mL), dried over Na₂SO₄, filtered, and concentrated under reduced pressure. The residue was purified by column to afford 5-bromo-2-((1-(difluoromethyl)-1H-pyrazol-4-yl)oxy)-3-fluoropyridine (1.6 g, 77% yield) as a white solid. LCMS (ESI) m/z=308.0 (M+H).

### Step 3: Preparation of 2-((1-(difluoromethyl)-1H-pyrazol-4-yl)oxy)-3-fluoro-5-(4,4,5,5-tetramethyl-1,3,2-dioxaborolan-2-yl)pyridine

A mixture of 5-bromo-2-((1-(difluoromethyl)-1H-pyrazol-4-yl)oxy)-3-fluoropyridine (500 mg, 1.62 mmol), 4,4,5,5-tetramethyl-2-(4,4,5,5-tetramethyl-1,3,2-dioxaborolan-2-yl)-1,3,2-dioxaborolane (618 mg, 2.43 mmol), Pd(dppf)Cl₂·CH₂Cl₂ (133 mg, 162 μmol), and potassium acetate (319 mg, 3.25 mmol) in dioxane (10 mL) was degassed and purged with nitrogen three times. The mixture was then stirred at 90° C. for 12 hr under a nitrogen atmosphere. The reaction mixture was filtered and the filtrate was concentrated to afford 2-((1-(difluoromethyl)-1H-pyrazol-4-yl)oxy)-3-fluoro-5-(4,4,5,5-tetramethyl-1,3,2-dioxaborolan-2-yl)pyridine (620 mg, 45% yield) as a yellow solid. LCMS (ESI) m/z=356.2 (M+H).

### Step 4: Preparation of methyl (R)-2-((1-(3-(6-((1-(difluoromethyl)-1H-pyrazol-4-yl)oxy)-5-fluoropyridin-3-yl)-7-fluoro-2-methyl-1-oxo-1,2-dihydroisoquinolin-5-yl)ethyl)amino)benzoate

A mixture of 2-((1-(difluoromethyl)-1H-pyrazol-4-yl)oxy)-3-fluoro-5-(4,4,5,5-tetramethyl-1,3,2-dioxaborolan-2-yl)pyridine (365 mg, 1.03 mmol), methyl (R)-2-((1-(3-chloro-7-fluoro-2-methyl-1-oxo-1,2-dihydroisoquinolin-5-yl)ethyl)amino)benzoate (200 mg, 514 μmol), Pd(dppf)Cl₂·CH₂Cl₂ (42 mg, 51.4 μmol), and K₂CO₃ (142 mg, 1.03 mmol) in dioxane (8 mL) and H₂O (1 mL) was degassed and purged with nitrogen three times. The mixture was then stirred at 90° C. for 12 hr under a nitrogen atmosphere. The reaction was quenched with water (10 mL) and extracted with DCM (3×15 mL). The combined organic layers were washed with brine (20 mL), dried over Na₂SO₄, filtered, and concentrated under reduced pressure. The residue was purified by column to afford methyl (R)-2-((1-(3-(6-((1-(difluoromethyl)-1H-pyrazol-4-yl)oxy)-5-fluoropyridin-3-yl)-7-fluoro-2-methyl-1-oxo-1,2-dihydroisoquinolin-5-yl)ethyl)amino)benzoate (220 mg, 73% yield) as yellow solid. LCMS (ESI) m/z=582.2 (M+H).

### Step 5: Preparation of (R)-2-((1-(3-(6-((1-(difluoromethyl)-1H-pyrazol-4-yl)oxy)-5-fluoropyridin-3-yl)-7-fluoro-2-methyl-1-oxo-1,2-dihydroisoquinolin-5-yl)ethyl)amino)benzoic acid

To a solution of methyl (R)-2-((1-(3-(6-((1-(difluoromethyl)-1H-pyrazol-4-yl)oxy)-5-fluoropyridin-3-yl)-7-fluoro-2-methyl-1-oxo-1,2-dihydroisoquinolin-5-yl)ethyl)amino)benzoate (210 mg, 361 μmol) in MeOH (1 mL), THF (1 mL) and H₂O (0.4 mL) was added LiOH·H₂O (45 mg, 1.08 mmol) at 60° C. for 1 hr. The reaction was quenched with water (2 mL), and the pH of the mixture was adjusted to 7 by adding an aqueous citric acid solution. The mixture was then extracted with EA (3×2 mL). The combined organic layers were washed with brine (5 mL), dried over Na₂SO₄, filtered, and concentrated under reduced pressure. The residue was purified by prep-HPLC and lyophilized to afford (R)-2-((1-(3-(6-((1-(difluoromethyl)-1H-pyrazol-4-yl)oxy)-5-fluoropyridin-3-yl)-7-fluoro-2-methyl-1-oxo-1,2-dihydroisoquinolin-5-yl)ethyl)amino)benzoic acid (29 mg, 14% yield) as white solid. LCMS (ESI) m/z=590.2 (M+Na); ¹H NMR (400 MHz, CDCl₃): δ 8.23 (s, 2H), 8.15 (d, J=2.0 Hz, 1H), 8.08-7.98 (m, 2H), 7.83 (s, 1H), 7.61-5.56 (m, 1H), 7.52-7.45 (m, 1H), 7.34-7.03 (m, 2H), 6.71 (s, 1H), 6.63 (t, J=7.6 Hz, 1H), 6.20 (d, J=8.4 Hz, 1H), 5.01-4.95 (m, 1H), 3.51 (s, 3H), 1.67 (d, J=6.8 Hz, 3H).

### Examples 49 to 595

Examples 49 to 595 were prepared in a manner similar to that described for Examples 1 to 48 and are identified and characterized in Table 1 below. If not specified otherwise, all depicted chiral centers exist as a (R)- and (S)-racemic mixture or as either (R)- or (S)-enantiomer.

TABLE 1
Examples 49 to 595

| | | | | ¹H NMR data |
|---|---|---|---|---|
| | | Structure | LCMS | (MHz, solvent, |
| Structure | Example | Comment | m/z | ppm) |
| 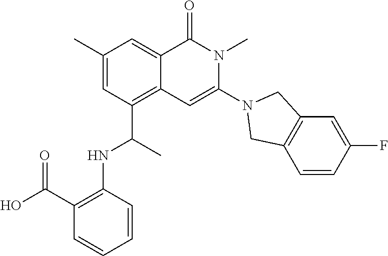 [SMILES: Cc1cc(C(C)Nc2ccccc2C(=O)O)c2cc(N3Cc4ccc(F)cc4C3)n(C)c(=O)c2c1] C₂₈H₂₆FN₃O₃ | 49 | enantiomer 1 | ES+ 472.1 [M + H] | ¹H NMR (300 MHz, Methanol-d₄) δ 7.98 (d, J = 1.7 Hz, 1H), 7.90-7.85 (m, 1H), 7.53 (d, J = 1.9 Hz, 1H), 7.32-7.28 (m, 1H), 7.19-6.95 (m, 3H), 6.72 (s, 1H), 6.51-6.50 (m, 1H), 6.38 (d, J = 8.5 Hz, 1H), 5.15-5.05 (m, 1H), 4.52 (d, J = 10.9 Hz, 4H), 3.68 (s, 3H), 2.37 (s, 3H), 1.62 (d, J = 6.7 Hz, 3H). |
| 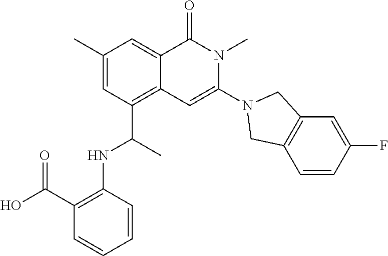 [SMILES: Cc1cc(C(C)Nc2ccccc2C(=O)O)c2cc(N3Cc4ccc(F)cc4C3)n(C)c(=O)c2c1] C₂₈H₂₆FN₃O₃ | 50 | enantiomer 2 | ES+ 472.0 [M + H] | ¹H NMR (300 MHz, Methanol-d₄) δ 7.98 (d, J = 1.9, 0.9 Hz, 1H), 7.89 (d, J = 8.0, 1.7 Hz, 1H), 7.52 (d, J = 1.9 Hz, 1H), 7.35- 7.28 (m, 1H), 7.19- 7.01 (m, 3H), 6.72 (s, 1H), 6.51-6.50 (m, 1H), 6.42-6.35 (m, 1H), 5.12-5.08 (m, 1H), 4.52 (d, J = 10.8 Hz, 4H), 3.68 (s, 3H), 2.37 (s, 3H), 1.62 (d, J = 6.7 Hz, 3H) |
| 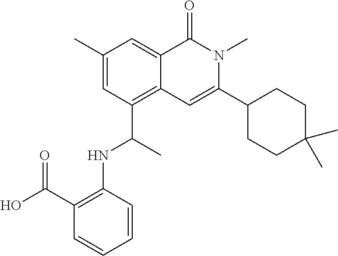 [SMILES: Cc1cc(C(C)Nc2ccccc2C(=O)O)c2cc(C3CCC(C)(C)CC3)n(C)c(=O)c2c1] C₂₈H₃₄N₂O₃ | 51 | enantiomer 1 | ES+ 447.0 [M + H] | ¹H NMR (300 MHz, Methanol-d₄) δ 8.01 (d, J = 1.7 Hz, 1H), 7.92-7.88 (m, 1H), 7.56 (d, J = 1.9 Hz, 1H), 7.17-7.15 (m, 1H), 6.81 (s, 1H), 6.59-6.40 (m, 2H), 5.20-5.10 (m, 1H), 3.65 (s, 3H), 2.75- 2.70 (m, 1H), 2.40 (s, 3H), 1.84-1.72 (m, 2H), 1.65 (d, J = 6.7 Hz, 3H), 1.62- 1.35 (m, 6H), 0.97 (d, J = 2.6 Hz, 6H). |
| 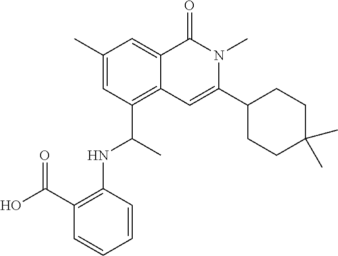 [SMILES: Cc1cc(C(C)Nc2ccccc2C(=O)O)c2cc(C3CCC(C)(C)CC3)n(C)c(=O)c2c1] C₂₈H₃₄N₂O₃ | 52 | enantiomer 2 | ES+ 447.0 [M + H] | ¹H NMR (300 MHz, Methanol-d₄) δ 8.01 (s, 1H), 7.90 (d, J = 8.0, 1.7 Hz, 1H), 7.56 (d, J = 1.9 Hz, 1H), 7.17-7.15 (m, 1H), 6.81 (s, 1H), 6.61-6.42 (m, 2H), 5.15-5.10 (m, 1H), 3.65 (s, 3H), 2.73- 2.68 (m, 1H), 2.40 (s, 3H), 1.84-1.72 (m, 2H), 1.65 (d, J = 6.7 Hz, 3H), 1.62- 1.34 (m, 6H), 0.97 (d, J = 2.5 Hz, 6H). |
| 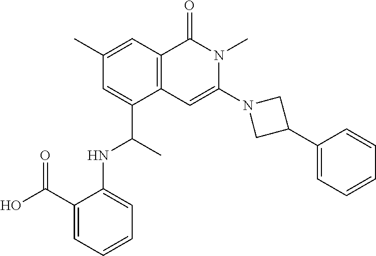 [SMILES: Cc1cc(C(C)Nc2ccccc2C(=O)O)c2cc(N3CC(c4ccccc4)C3)n(C)c(=O)c2c1] C₂₉H₂₉N₃O₃ | 53 | | ES+ 468.1 [M + H] | ¹H NMR (400 MHz, DMSO-d₆) δ 8.56 (s, 1H), 7.81 (d, J = 8.2 Hz, 2H), 7.48-7.33 (m, 5H), 7.32-7.22 (m, 1H), 7.20-7.07 (m, 1H), 6.50-6.47 (m, 1H), 6.37 (d, J = 8.5 Hz, 1H), 5.96 (s, 1H), 5.11-5.09 (m, 1H), 4.37-4.32 (m, 2H), 4.07-3.84 (m, 3H), 3.46 (s, 3H), 2.29 (s, 3H), 1.53 (d, J = 6.5 Hz, 3H). |
| 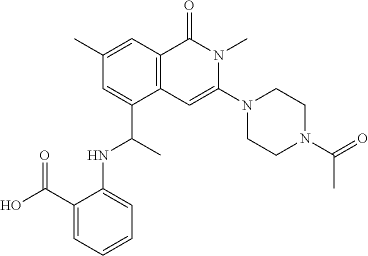 [SMILES: CC(=O)N1CCN(c2cc3c(C(C)Nc4ccccc4C(=O)O)cc(C)cc3c(=O)n2C)CC1] C₂₆H₃₀N₄O₄ | 54 | | ES+ 463.2 [M + H] | ¹H NMR (400 MHz, Methanol-d₄, ppm) 8 8.00-7.95 (m, 1H), 7.90-7.88 (m, 1H), 7.54 (d, J = 1.9 Hz, 1H), 7.17-6.99 (m, 1H), 6.55-6.47 (m, 2H), 6.40-6.35 (m, 1H), 5.12-5.08 (m, 1H), 4.53-4.50 (m, 1H), 3.98-6.84 (m, 1H), 3.66 (s, 3H), 3.54-3.51 (m, 1H), 3.22-3.18 (m, 2H), 2.99 (m, 1H), 2.82-2.78 (m, 2H), 2.37 (s, 3H), 2.16 (s, 3H), 1.63 (d, J = 6.7 Hz, 3H). |
| 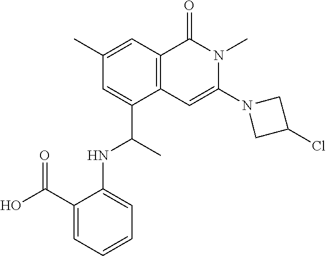 [SMILES: Cc1cc(C(C)Nc2ccccc2C(=O)O)c2cc(N3CC(Cl)C3)n(C)c(=O)c2c1] C₂₃H₂₄ClN₃O₃ | 55 | | ES+ 426.1 [M + H] | ¹H NMR (300 MHz, DMSO-d₆, ppm) 8 12.72 (s, 1H), 8.40 (s, 1H), 7.80 (d, J = 8.0, 1.7 Hz, 2H), 7.37 (d, J = 1.9 Hz, 1H), 7.28-7.19 (m, 1H), 6.57-6.46 (m, 1H), 6.36 (d, J = 8.4 Hz, 1H), 5.93 (s, 1H), 5.14-5.09 (m, 1H), 4.99-4.95 (m, 1H), 4.52-4.45 (m, 2H), 4.13-3.92 (m, 2H), 3.40 (s, 3H), 2.29 (s, 3H), 1.52 (d, J = 6.4 Hz, 3H). |
| 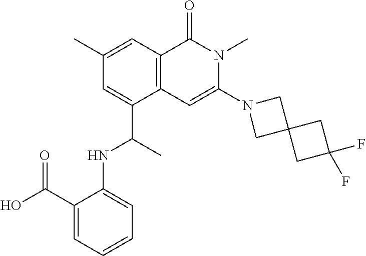 [SMILES: Cc1cc(C(C)Nc2ccccc2C(=O)O)c2cc(N3CC4(C3)CC(F)(F)C4)n(C)c(=O)c2c1] C₂₆H₂₇F₂N₃O₃ | 56 | | ES+ 468.5 [M + H] | ¹H NMR (400 MHz, DMSO-d₆) δ 12.68 (s, 1H), 8.39 (s, 1H), 7.84-7.77 (m, 2H), 7.35 (d, J = 1.9 Hz, 1H), 7.20-7.17 (m, 1H), 6.56-6.47 (m, 1H), 6.35 (d, J = 8.4 Hz, 1H), 5.85 (s, 1H), 5.08 (d, J = 7.2 Hz, 1H), 4.09-3.99 (m, 4H), 3.40 (s, 3H), 2.88 (t, J = 12.5 Hz, 4H), 2.28 (s, 3H), 1.53 (d, J = 6.5 Hz, 3H). |
| 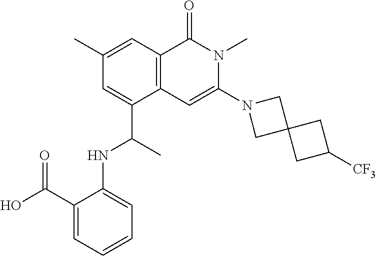 [SMILES: Cc1cc(C(C)Nc2ccccc2C(=O)O)c2cc(N3CC4(CC(C(F)(F)F)C4)C3)n(C)c(=O)c2c1] C₂₇H₂₈F₃N₃O₃ | 57 | | ES+ 500.1 [M + H] | ¹H NMR (400 MHz, DMSO-d₆) δ 8.73 (s, 1H), 7.84-7.76 (m, 2H), 7.36 (d, J = 1.9 Hz, 1H), 7.12- 7.11 (m, 1H), 6.52- 6.44 (m, 1H), 6.29 (d, J = 8.4 Hz, 1H), 5.82 (s, 1H), 5.04 (q, J = 6.6 Hz, 1H), 4.04- 3.88 (m, 4H), 3.39 (s, 3H), 3.20-3.05 (m, 1H), 2.54 (m, 1H), 2.49 (m, 1H), 2.31 (m, 6H), 1.51 (d, J = 6.5 Hz, 3H). |
| 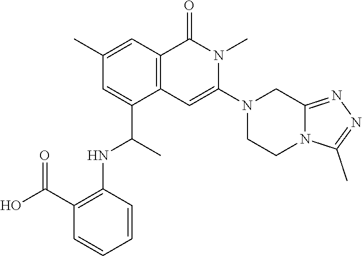 [SMILES: Cc1cc(C(C)Nc2ccccc2C(=O)O)c2cc(N3CCn4c(C)nnc4C3)n(C)c(=O)c2c1] C₂₆H₂₈N₆O₃ | 58 | | ES+ 473.6 [M + H] | ¹H NMR (400 MHz, Methanol-d₄) δ 8.03- 7.98 (m, 1H), 7.89- 7.85 (m, 1H), 7.59- 7.54 (m, 1H), 7.13- 7.10 (m, 1H), 6.70 (s, 1H), 6.51 (d, J = 7.5 Hz, 1H), 6.38 (d, J = 8.5 Hz, 1H), 5.11- 5.10 (m, 1H), 4.43 (s, 2H), 4.16 (s, 2H), 3.66 (s, 3H), 3.31 (d, J = 1.6 Hz, 2H), 2.46 (s, 3H), 2.39 (s, 3H), 1.66-1.60 (m, 3H). |
| 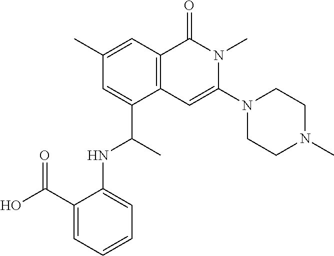 [SMILES: Cc1cc(C(C)Nc2ccccc2C(=O)O)c2cc(N3CCN(C)CC3)n(C)c(=O)c2c1] C₂₅H₃₀N₄O₃ | 59 | | ES+ 435.5 [M + H] | ¹H NMR (300 MHz, DMSO-d₆) δ 8.46 (s, 1H), 7.87 (s, 1H), 7.81-7.79 (m, 1H), 7.42 (d, J = 1.9 Hz, 1H), 7.24-7.12 (m, 1H), 6.51-6.48 (m, 1H), 6.43-6.32 (m, 2H), 5.13 (s, 1H), 3.91 (m, 4H), 3.48 (s, 3H), 2.95 (m, 4H), 2.29 (s, 3H), 2.26 (s, 3H), 1.54 (d, J = 6.5 Hz, 3H). |
| 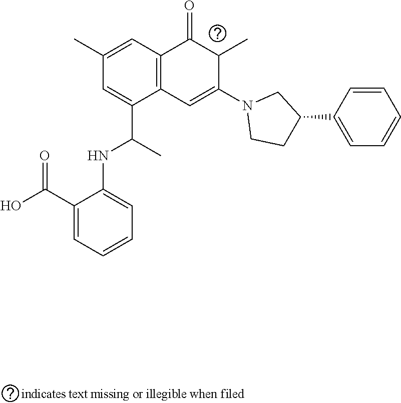 [SMILES: Cc1cc2c(c(C(C)Nc3ccccc3C(=O)O)c1)C=C(N1CC[C@@H](c3ccccc3)C1)C(C)C2=O] C₃₀H₃₁N₃O₃ | 60 | mixture of diastereomers | ES+ 482.6 [M + H] | ¹H NMR (400 MHz, Methanol-d₄) δ 7.93- 7.96 (m, 1H), 7.90- 7.87 (m, 1H), 7.51- 7.49 (m, 1H), 7.40- 7.27 (m, 5H), 7.30- 7.17 (m, 1H), 7.15- 7.12 (m, 1H), 6.50- 6.48 (m, 1H), 6.44- 6.37 (m, 1H), 5.12- 5.02 (m, 1H), 3.62 (s, 3H), 3.60-3.50 (m, 1H), 3.53-3.39 (m, 2H), 3.27-3.25 (m, 2H), 2.45- 2.425 (m, 1H), 2.36 (s, 3H), 2.13-2.11 (m, 1H), 1.65-1.62 (m, 3H). |
| 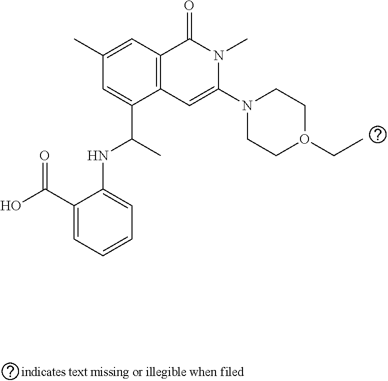 C₂₆H₂₉F₃N₄O₃ | 61 | | ES+ 503.4 [M + H] | ¹H NMR (400 MHz, DMSO-d₆) δ 12.75 (s, 1H), 8.41 (s, 1H), 7.80-7.85 (m, 1H), 7.83-7.77 (m, 1H), 7.42 (s, 1H), 7.23- 7.15 (m, 1H), 6.56- 6.48 (m, 1H), 6.43- 6.35 (m, 2H), 5.16 (m, 1H), 3.49 (s, 3H), 3.40 (m, 1H), 3.32 (m, 2H), 3.35- 3.31 (m, 1H), 3.27 (m, 1H), 3.27-3.24 (m, 1H), 3.12-2.64 (m, 4H), 2.32 (s, 3H), 1.55-1.51 (m, 3H). |
| 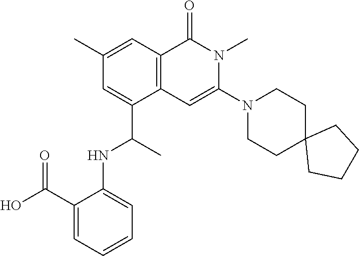 [SMILES: Cc1cc(C(C)Nc2ccccc2C(=O)O)c2cc(N3CCC4(CCCC4)CC3)n(C)c(=O)c2c1] C₂₉H₃₅N₃O₃ | 62 | | ES+ 474.6 [M + H] | ¹H NMR (300 MHz, DMSO-d₆) δ 12.68 (s, 1H), 8.34 (d, J = 5.9 Hz, 1H), 7.87 (d, J = 1.5 Hz, 1H), 7.81- 7.79 (m, 1H), 7.40 (d, J = 1.9 Hz, 1H), 7.22-7.20 (m, 1H), 6.58-6.47 (m, 1H), 6.40 (d, J = 8.4 Hz, 1H), 6.33 (s, 1H), 5.19-5.09 (m, 1H), 3.48 (s, 3H), 2.89 (s, 3H), 2.32 (s, 3H), 1.61-1.53 (m, 16H). |
| 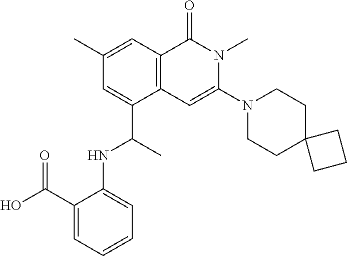 [SMILES: Cc1cc(C(C)Nc2ccccc2C(=O)O)c2cc(N3CCC4(CCC4)CC3)n(C)c(=O)c2c1] C₂₈H₃₃N₃O₃ | 63 | | ES+ 460.6 [M + H] | ¹H NMR (300 MHz, DMSO-d₆) δ 12.72 (s, 1H), 8.35 (d, J = 5.8 Hz, 1H), 7.87- 7.85 (m, 1H), 7.81- 7.79 (m, 1H), 7.40 (d, J = 1.9 Hz, 1H), 7.21-7.19 (m, 1H), 6.53-6.51 (m, 1H), 6.43-6.34 (m, 1H), 6.29 (s, 1H), 5.17- 5.07 (m, 1H), 3.47 (s, 3H), 3.17-2.51 (m, 4H), 2.32 (s, 3H), 1.97-1.66 (m, 10H), 1.54 (d, J = 6.5 Hz, 3H). |
| 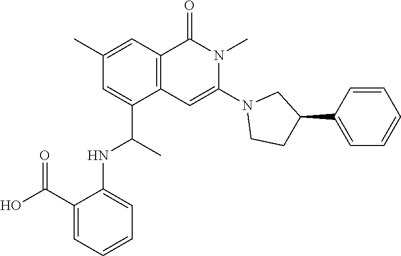 [SMILES: Cc1cc(C(C)Nc2ccccc2C(=O)O)c2cc(N3CC[C@H](c4ccccc4)C3)n(C)c(=O)c2c1] C₃₀H₃₁N₃O₃ | 64 | | ES+ 482.6 [M + H] | ¹H NMR (300 MHz, DMSO-d₆) δ 12.73 (s, 1H), 8.35 (d, J = 5.9 Hz, 1H), 7.88- 7.74 (m, 2H), 7.43- 7.30 (m, 5H), 7.22- 7.20 (m, 2H), 6.49- 6.51 (m, 1H), 6.42- 6.39 (m, 1H), 6.30 (s, 1H), 5.20-5.10 (m, 1H), 3.50-3.45 (m, 6H), 3.37-3.19 (m, 3H), 2.38 (s, 1H), 2.31 (s, 3H), 1.55-1.53 (m, 3H). |
| 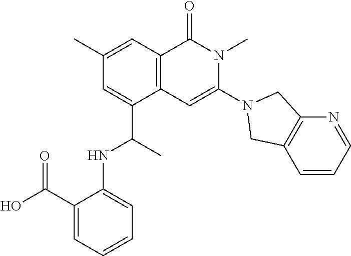 [SMILES: Cc1cc(C(C)Nc2ccccc2C(=O)O)c2cc(N3Cc4cccnc4C3)n(C)c(=O)c2c1] C₂₇H₂₆N₄O₃ | 65 | | ES+ 455.2 [M + H] | ¹H NMR (300 MHz, DMSO-d₆, ppm) 8 13.35-12.00 (br s, 1H), 8.56-8.44 (m, 2H), 7.88 (d, J = 1.7 Hz, 1H), 7.80 (d, J = 7.9, 1.7 Hz, 2H), 7.41 (d, J = 1.9 Hz, 1H), 7.35-7.28 (m, 1H), 7.21-7.09 (m, 1H), 6.74 (s, 1H), 6.52-6.48 (m, 1H), 6.36 (d, J = 8.4 Hz, 1H), 5.21 (m, 1H), 4.76-4.53 (m, 4H), 3.59 (s, 3H), 2.32 (s, 3H), 1.52 (d, J = 6.4 Hz, 3H). |
| 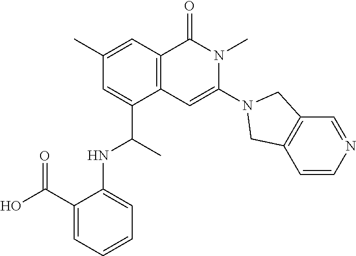 [SMILES: Cc1cc(C(C)Nc2ccccc2C(=O)O)c2cc(N3Cc4ccncc4C3)n(C)c(=O)c2c1] C₂₇H₂₆N₄O₃ | 66 | | ES+ 455.1 [M + H] | ¹H NMR (300 MHz, DMSO-d₆) δ 8.60 (s, 1H), 8.51 (d, J = 5.0 Hz, 1H), 7.88 (s, 1H), 7.80 (d, J = 7.7 Hz, 1H), 7.48-7.39 (m, 2H), 7.15-7.11 (m, 1H), 6.71 (s, 1H), 6.51-6.47 (m, 1H), 6.33 (d, J = 8.5 Hz, 1H), 5.17 (m, 1H), 4.74-4.56 (m, 4H), 3.57 (s, 4H), 2.32 (s, 3H), 1.51 (d, J = 6.4 Hz, 3H). |
| 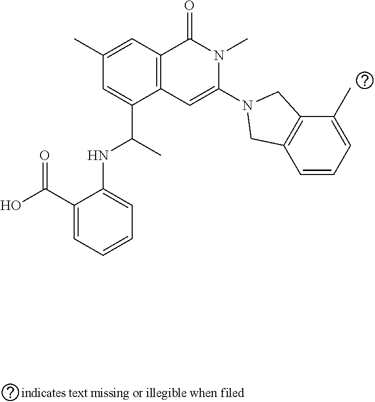 C₂₉H₂₆F₃N₃O₃ | 67 | | ES+ 522.1 [M + H] | ¹H NMR (300 MHz, Methanol-d₄) δ 7.89 (s, 1H), 7.77 (d, J = 8.0, 1.7 Hz, 1H), 7.50-7.34 (m, 5H), 7.08-6.96 (m, 1H), 6.43-6.38 (m, 1H), 6.28 (d, J = 8.5 Hz, 1H), 5.02-4.98 (m, 1H), 4.60-4.45 (m, 4H), 3.60 (s, 3H), 2.27 (s, 3H), 1.51 (d, J = 6.7 Hz, 3H). |
| 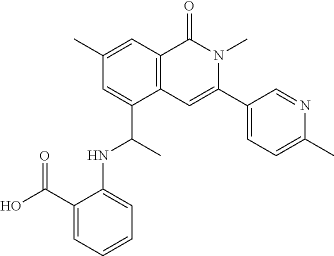 [SMILES: Cc1cc(C(C)Nc2ccccc2C(=O)O)c2cc(-c3ccc(C)nc3)n(C)c(=O)c2c1] C₂₆H₂₅N₃O₃ | 68 | | ES− 425.9 [M − H] | ¹H NMR (400 MHz, DMSO-d₆, ppm) δ 12.82 (s, 1H), 8.67 (d, J = 2.3 Hz, 1H), 8.55 (s, 1H), 7.99 (s, 1H), 7.96-7.91 (m, 1H), 7.82-7.79 (m, 1H), 7.50 (d, J = 1.9 Hz, 1H), 7.42 (d, J = 8.0 Hz, 1H), 7.16- 7.13 (m, 1H), 6.90 (s, 1H), 6.52-6.48 (m, 1H), 6.32 (d, J = 8.5 Hz, 1H), 5.21 (m, 1H), 3.35 (s, 3H), 2.56 (s, 3H), 2.37 (s, 3H), 1.50 (d, J = 6.5 Hz, 3H). |
| 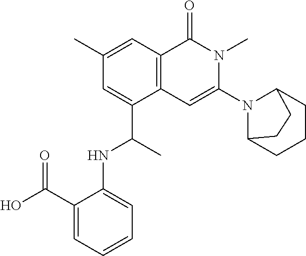 [SMILES: Cc1cc(C(C)Nc2ccccc2C(=O)O)c2cc(N3C4CCCC3CC4)n(C)c(=O)c2c1] C₂₇H₃₁N₃O₃ | 69 | | ES+ 446.6 [M + H] | ¹H NMR (300 MHz, DMSO-d₆) δ 8.39 (s, 1H), 7.87-7.75 (m, 2H), 7.40 (d, J = 1.9 Hz, 1H), 7.20-7.17 (m, 1H), 6.53-6.50 (m, 1H), 6.44 (d, J = 8.5 Hz, 1H), 6.11 (s, 1H), 5.12-5.08 (m, 1H), 3.80-3.75 (m, 2H), 3.56 (s, 3H), 2.32 (s, 3H), 2.14- 1.62 (m, 7H), 1.54 (d, J = 6.5 Hz, 6H). |
| 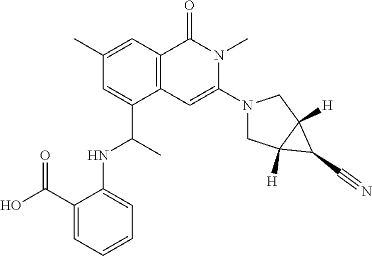 [SMILES: Cc1cc(C(C)Nc2ccccc2C(=O)O)c2cc(N3C[C@@H]4[C@@H](C#N)[C@@H]4C3)n(C)c(=O)c2c1] C₂₆H₂₆N₄O₃ | 70 | | ES+ 443.2 [M + H] | ¹H NMR (400 MHz, Methanol-d₄) δ 7.97 (s, 1H), 7.92 (d, J = 8.0, 1.7 Hz, 1H), 7.54 (d, J = 1.8 Hz, 1H), 7.18-7.14 (m, 1H), 6.59 (s, 1H), 6.56-6.53 (m, 1H), 6.37 (d, J = 8.5 Hz, 1H), 5.12-5.09 (m, 1H), 3.55-3.51 (m, 2H), 3.52 (s, 3H), 3.25-3.18 (m, 2H), 2.34-2.22 (m, 5H), 2.02-2.06 (m, 1H), 1.64 (d, J = 6.7 Hz, 3H). |
| 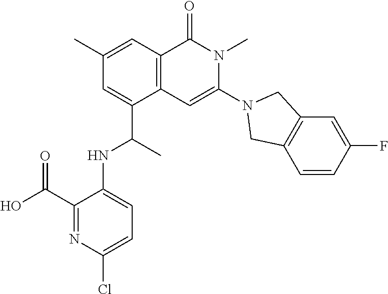 [SMILES: Cc1cc(C(C)Nc2ccc(Cl)nc2C(=O)O)c2cc(N3Cc4ccc(F)cc4C3)n(C)c(=O)c2c1] C₂₇H₂₄ClFN₄O₃ | 71 | | ES+ 507.2 [M + H] | ¹H NMR (Methanol- d4, 300 MHz, ppm): 8 8.00 (d, J = 1.4 Hz, 1H), 7.52 (d, J = 1.8 Hz, 1H), 7.42-7.31 (m, 1H), 7.21-6.98 (m, 3H), 6.83-6.75 (m, 2H), 5.19-5.07 (m, 1H), 4.57 (d, J = 11.2 Hz, 4H), 3.70 (s, 3H), 2.38 (s, 3H), 1.64 (d, J = 6.7 Hz, 3H). |
| 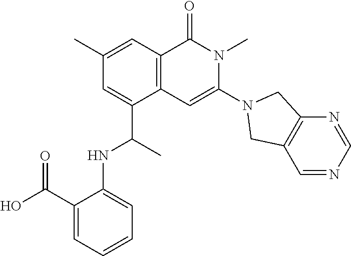 [SMILES: Cc1cc(C(C)Nc2ccccc2C(=O)O)c2cc(N3Cc4cncnc4C3)n(C)c(=O)c2c1] C₂₆H₂₅N₅O₃ | 72 | | ES+ 456.1 [M + H] | ¹H NMR (400 MHz, DMSO-d₆) δ 9.13 (s, 1H), 8.82 (s, 1H), 8.54 (s, 1H), 7.89 (s, 1H), 7.80 (d, J = 8.0 Hz, 1H), 7.42 (s, 1H), 7.16 (s, 1H), 6.79 (s, 1H), 6.50 (s, 1H), 6.34 (d, J = 8.6 Hz, 1H), 5.25-5.20 (m, 1H), 4.75-4.66 (m, 4H), 3.58 (s, 3H), 2.32 (s, 3H), 1.52 (d, J = 6.3 Hz, 3H). |
| 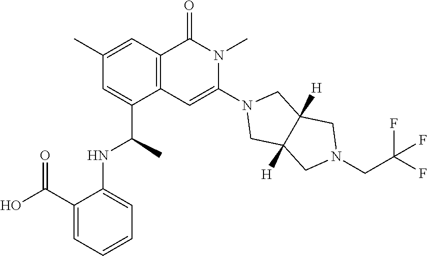 [SMILES: Cc1cc([C@@H](C)Nc2ccccc2C(=O)O)c2cc(N3C[C@H]4CN(CC(F)(F)F)C[C@H]4C3)n(C)c(=O)c2c1] C₂₈H₃₁F₃N₄O₃ | 73 | | ES+ 529.2 [M + H] | ¹H NMR (DMSO-d₆, 400 MHz, ppm): δ 8.67 (s, 1H), 7.92- 7.74 (m, 2H), 7.42 (s, 1H), 7.14 (d, J = 7.8 Hz, 1H), 6.52- 6.48 (m, 1H), 6.37- 6.29 (m, 2H), 5.12- 5.08 (m, 1H), 3.52 (s, 3H), 3.30-3.19 (m, 2H), 3.08-3.05 (m, 2H), 3.03-2.89 (m, 4H), 2.84 (m, 2H), 2.59-2.52 (m, 2H), 2.32 (s, 3H), 1.53 (d, J = 6.5 Hz, 3H). |
| 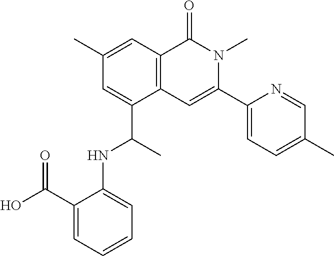 [SMILES: Cc1ccc(-c2cc3c(C(C)Nc4ccccc4C(=O)O)cc(C)cc3c(=O)n2C)nc1] C₂₆H₂₅N₃O₃ | 74 | | ES+ 428.2 [M + H] | ¹H NMR (400 MHz, DMSO-d₆) δ 8.67 (s, 1H), 8.58 (d, J = 2.2 Hz, 1H), 8.00 (d, J = 1.7 Hz, 1H), 7.85- 7.77 (m, 2H), 7.67 (d, J = 7.9 Hz, 1H), 7.51 (d, J = 1.9 Hz, 1H), 7.16-7.07 (m, 1H), 6.97 (s, 1H), 6.53-6.44 (m, 1H), 6.30 (d, J = 8.4 Hz, 1H), 5.19 (m, 1H), 3.35 (s, 3H), 2.39 (d, J = 8.3 Hz, 6H), 1.51 (d, J = 6.5 Hz, 3H). |
| 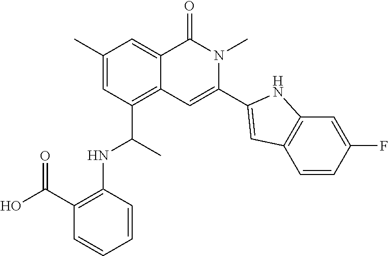 [SMILES: Cc1cc(C(C)Nc2ccccc2C(=O)O)c2cc(-c3cc4ccc(F)cc4[nH]3)n(C)c(=O)c2c1] C₂₈H₂₄FN₃O₃ | 75 | | ES+ 470.2 [M + H] | ¹H NMR (300 MHz, Methanol-d₄) δ 8.12- 8.05 (m, 1H), 7.90- 7.87 (m, 1H), 7.65- 7.54 (m, 2H), 7.20 (s, 1H), 7.18-7.03 (m, 2H), 6.90-6.86 (m, 1H), 6.78 (d, J = 0.9 Hz, 1H), 6.57- 6.45 (m, 1H), 6.30 (d, J = 8.1 Hz, 1H), 5.20-5.16 (m, 1H), 3.64 (s, 3H), 2.41 (s, 3H), 1.62 (d, J = 6.7 Hz, 3H). |
| 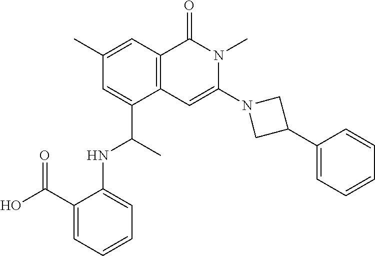 [SMILES: Cc1cc(C(C)Nc2ccccc2C(=O)O)c2cc(N3CC(c4ccccc4)C3)n(C)c(=O)c2c1] C₂₉H₂₉N₃O₃ | 76 | enantiomer 1 | ES+ 468.1 [M + H] | ¹H NMR (400 MHz, DMSO-d₆) δ 8.42- 8.36 (m, 1H), 7.85- 7.76 (m, 2H), 7.46- 7.34 (m, 5H), 7.28 (d, J = 7.2 Hz, 1H), 7.22-7.16 (m, 1H), 6.52-6.48 (m, 1H), 6.39 (d, J = 8.5 Hz, 1H), 5.96 (s, 1H), 5.15-5.09 (m, 1H), 4.40-4.35 (m, 2H), 3.97-3.93 (m, 3H), 3.46 (s, 3H), 2.30 (s, 3H), 1.54 (d, J = 6.5 Hz, 3H). |
| 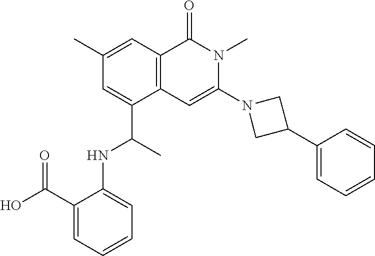 [SMILES: Cc1cc(C(C)Nc2ccccc2C(=O)O)c2cc(N3CC(c4ccccc4)C3)n(C)c(=O)c2c1] C₂₉H₂₉N₃O₃ | 77 | enantiomer 2 | ES+ 468.1 [M + H] | ¹H NMR (400 MHz, DMSO-d₆) δ 8.41 (s, 1H), 7.85-7.76 (m, 2H), 7.41 (d, J = 15.3, 7.9 Hz, 5H), 7.30-7.26 (m, 1H), 7.22-7.16 (m, 1H), 6.52-6.48 (m, 1H), 6.39 (d, J = 8.5 Hz, 1H), 5.96 (s, 1H), 5.16-5.08 (m, 1H), 4.34-4.32 (m, 2H), 3.99-3.91 (m, 3H), 3.46 (s, 3H), 2.30 (s, 3H), 1.54 (d, J = 6.5 Hz, 3H). |
| 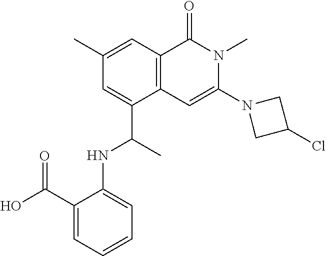 [SMILES: Cc1cc(C(C)Nc2ccccc2C(=O)O)c2cc(N3CC(Cl)C3)n(C)c(=O)c2c1] C₂₃H₂₄ClN₃O₃ | 78 | enantiomer 1 | ES+ 426.1 [M + H] | ¹H NMR (DMSO-d₆, 400 MHz, ppm): 12.72 (s, 1H), 8.40 (s, 1H), 7.80-7.78 (m, 2H), 7.40-7.35 (m, 1H), 7.28-7.19 (m, 1H), 6.57-6.46 (m, 1H), 6.36 (d, J = 8.4 Hz, 1H), 5.93 (s, 1H), 5.18-5.12 (m, 1H), 4.99-4.99 (m, 1H), 4.50-4.45(m, 2H), 4.13-3.92 (m, 2H), 3.40 (s, 3H), 2.29 (s, 3H), 1.52 (d, J = 6.4 Hz, 3H). |
| 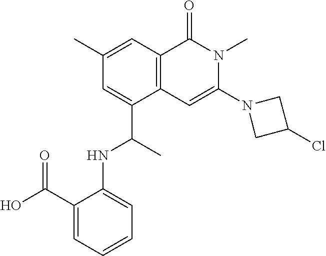 [SMILES: Cc1cc(C(C)Nc2ccccc2C(=O)O)c2cc(N3CC(Cl)C3)n(C)c(=O)c2c1] C₂₃H₂₄ClN₃O₃ | 79 | enantiomer 2 | ES+ 426.1 [M + H] | ¹H NMR (DMSO-d₆, 400 MHz, ppm): 8.40 (s, 1H), 7.80- 7.78 (m, 2H), 7.37 (d, J = 1.9 Hz, 1H), 7.28-7.19 (m, 1H), 6.57-6.46 (m, 1H), 6.36 (d, J = 8.4 Hz, 1H), 5.93 (s, 1H), 5.18-5.12 (m, 1H), 4.91-4.84 (m, 1H), 4.51-4.45 (m, 2H), 4.13-3.92 (m, 2H), 3.40 (s, 3H), 2.29 (s, 3H), 1.52 (d, J = 6.4 Hz, 3H). |
| 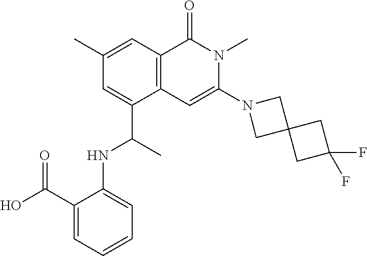 [SMILES: Cc1cc(C(C)Nc2ccccc2C(=O)O)c2cc(N3CC4(C3)CC(F)(F)C4)n(C)c(=O)c2c1] C₂₆H₂₇F₂N₃O₃ | 80 | enantiomer 1 | ES+ 468.5 [M + H] | ¹H NMR (300 MHz, DMSO-d₆) δ 8.45 (s, 1H), 7.78-7.81 (m, 2H), 7.35 (d, J = 1.9 Hz, 1H), 7.18-7.11 (m, 1H), 6.53-6.50 (m, 1H), 6.33 (d, J = 8.4 Hz, 1H), 5.85 (s, 1H), 5.10-5.05 (m, 1H), 4.11-3.98 (m, 4H), 3.40 (s, 3H), 2.90-2.85 (m, 4H), 2.28 (s, 3H), 1.53 (d, J = 6.5 Hz, 3H). |
| 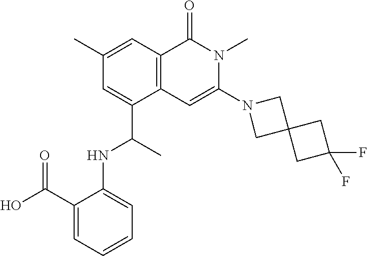 [SMILES: Cc1cc(C(C)Nc2ccccc2C(=O)O)c2cc(N3CC4(C3)CC(F)(F)C4)n(C)c(=O)c2c1] C₂₆H₂₇F₂N₃O₃ | 81 | enantiomer 2 | ES+ 468.5 [M + H] | ¹H NMR (300 MHz, DMSO-d₆) δ 8.48 (s, 1H), 7.85-7.76 (m, 2H), 7.35 (d, J = 1.9 Hz, 1H), 7.19-7.15 (m, 1H), 6.52-6.50 (m, 1H), 6.33 (d, J = 8.4 Hz, 1H), 5.85 (s, 1H), 5.10-5.05 (m, 1H), 4.11-3.98 (m, 4H), 3.40 (s, 3H), 2.85-2.90 (m, 4H), 2.28 (s, 3H), 1.53 (d, J = 6.5 Hz, 3H). |
| 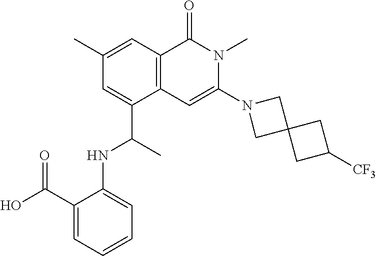 [SMILES: Cc1cc(C(C)Nc2ccccc2C(=O)O)c2cc(N3CC4(CC(C(F)(F)F)C4)C3)n(C)c(=O)c2c1] C₂₇H₂₈F₃N₃O₃ | 82 | enantiomer 1 | ES+ 500.1 [M + H] | ¹H NMR (400 MHz, DMSO-d₆) δ 12.73 (s, 1H), 8.45 (s, 1H), 7.80-7.78 (m, 2H), 7.35 (d, J = 1.9 Hz, 1H), 7.22-7.17 (m, 1H), 6.52-6.48 (m, 1H), 6.34 (d, J = 8.5 Hz, 1H), 5.81 (s, 1H), 5.11-5.03 (m, 1H), 4.04-3.88 (m, 4H), 3.39 (s, 3H), 3.15-3.11 (m, 1H), 2.54 (m, 2H), 2.38- 2.30 (m, 2H), 2.28 (s, 3H), 1.52 (d, J = 6.5 Hz, 3H). |
| 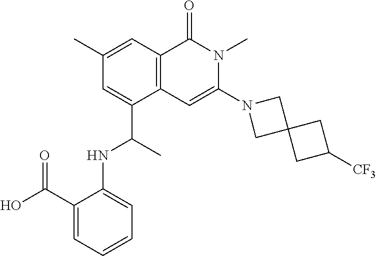 [SMILES: Cc1cc(C(C)Nc2ccccc2C(=O)O)c2cc(N3CC4(CC(C(F)(F)F)C4)C3)n(C)c(=O)c2c1] C₂₇H₂₈F₃N₃O₃ | 83 | enantiomer 2 | ES+ 500.1 [M + H] | ¹H NMR (400 MHz DMSO-d₆) δ 12.73 (s, 1H), 8.41 (s, 1H), 7.80-7.78 (m, 2H), 7.35 (d, J = 1.9 Hz, 1H), 7.23-7.14 (m, 1H), 6.55-6.49 (m, 1H), 6.34 (d, J = 8.5 Hz, 1H), 5.81 (s, 1H), 5.11-5.04 (m, 1H), 4.08-3.84 (m, 4H), 3.39 (s, 3H), 3.15-3.11 (m, 1H), 2.54 (m, 2H), 2.38- 2.30 (m, 2H), 2.28 (s, 3H), 1.53 (d, J = 6.6 Hz, 3H). |
| 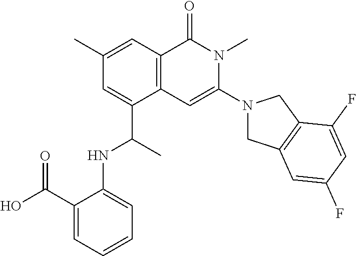 [SMILES: Cc1cc(C(C)Nc2ccccc2C(=O)O)c2cc(N3Cc4cc(F)cc(F)c4C3)n(C)c(=O)c2c1] C₂₈H₂₅F₂N₃O₃ | 84 | | ES+ 490.0 [M + H] | ¹H NMR (300 MHz, DMSO-d₆) δ 12.72 (s, 1H), 8.40 (d, J = 5.8 Hz, 1H), 7.89 (d, J = 1.9 Hz, 1H), 7.85- 7.76 (m, 1H), 7.41 (d, J = 1.9 Hz, 1H), 7.27-7.16 (m, 1H), 7.22-7.11 (m, 2H), 6.74 (s, 1H), 6.58- 6.47 (m, 1H), 6.37 (d, J = 8.5 Hz, 1H), 5.29-5.18 (m, 1H), 4.74-4.56 (m, 4H), 3.34 (s, 3H), 2.33 (s, 3H), 1.52 (d, J = 6.4 Hz, 3H). |
| 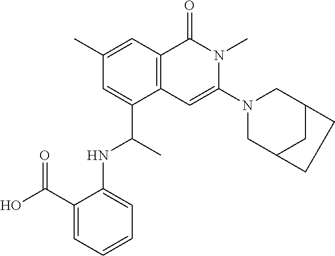 [SMILES: Cc1cc(C(C)Nc2ccccc2C(=O)O)c2cc(N3CC4CCC(C4)C3)n(C)c(=O)c2c1] C₂₇H₃₁N₃O₃ | 85 | | ES+ 446.6 [M + H] | ¹H NMR (300 MHz, DMSO-d₆) δ 8.75 (s, 1H), 7.85 (s, 1H), 7.80-7.79 (m, 1H), 7.42 (d, J = 1.9 Hz, 1H), 7.12-7.10 (m, 1H), 6.53-6.42 (m, 2H), 6.30 (d, J = 8.4 Hz, 1H), 5.10-5.06 (m, 1H), 3.56 (s, 3H), 2.97-2.95 (m, 2H), 2.79-2.77 (m, 2H), 2.29 (m, 5H), 1.82 (d, J = 7.7 Hz, 2H), 1.77-1.62 (m, 2H), 1.60-1.44 (m, 5H). |
| 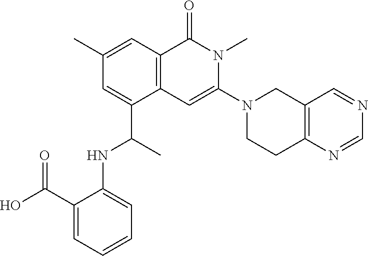 [SMILES: Cc1cc(C(C)Nc2ccccc2C(=O)O)c2cc(N3CCc4ncncc4C3)n(C)c(=O)c2c1] C₂₇H₂₇N₅O₃ | 86 | | ES+ 470.5 [M + H] | ¹H NMR (300 MHz, DMSO-d₆) δ 9.01 (s, 1H), 8.63 (s, 1H), 8.41 (d, J = 6.0 Hz, 1H), 7.90 (d, J = 1.5 Hz, 1H), 7.82-7.80 (m, 1H), 7.44 (d, J = 1.9 Hz, 1H), 7.22- 7.20 (m, 1H), 6.59- 6.48 (m, 2H), 6.39 (d, J = 8.6 Hz, 1H), 5.23-5.13 (m, 1H), 4.28 (m, 2H), 3.52 (s, 3H), 3.49-2.72 (m, 4H), 2.34 (s, 3H), 1.56 (d, J = 6.4 Hz, 3H). |
| 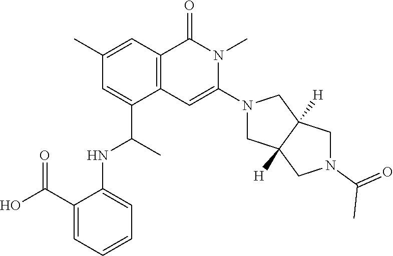 [SMILES: CC(=O)N1C[C@@H]2CN(c3cc4c(C(C)Nc5ccccc5C(=O)O)cc(C)cc4c(=O)n3C)C[C@H]2C1] C₂₈H₃₂N₄O₄ | 87 | | ES− 487.3 [M − H] | ¹H NMR (300 MHz, DMSO-d₆) δ 13.5- 12.0 (m, 1H), 8.42 (s, 1H), 7.90-7.74 (m, 2H), 7.38-7.28 (m, 1H), 7.26-7.12 (m, 1H), 6.55-6.40 (m, 1H), 6.38-6.30 (m, 1H), 6.21 (d, J = 3.9 Hz, 1H), 5.14- 5.02 (m, 1H), 3.71- 3.60 (m, 2H), 3.46 (s, 3H), 3.39 (m, 1H), 3.28-3.15 (m, 3H), 2.99-2.88 (m, 1H), 2.29 (d, J = 5.5 Hz, 6H), 1.95 (s, 3H), 1.53-1.50 (m, 3H). |
| 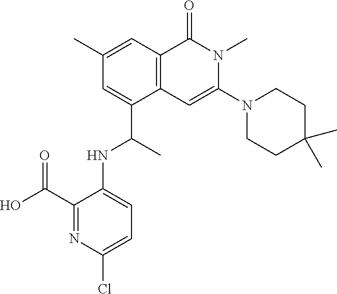 [SMILES: Cc1cc(C(C)Nc2ccc(Cl)nc2C(=O)O)c2cc(N3CCC(C)(C)CC3)n(C)c(=O)c2c1] C₂₆H₃₁ClN₄O₃ | 88 | | ES+ 483.2 [M + H] | ¹H NMR (300 MHz, Methanol-d₄) δ 7.95 (s, 1H), 7.54 (d, J = 1.5 Hz, 1H), 7.00- 6.97 (m, 1H), 6.69 (d, J = 8.7 Hz, 1H), 6.48 (s, 1H), 5.09- 5.01 (m, 1H), 3.61 (s, 3H), 2.98 (m, 4H), 2.39 (s, 3H), 1.85-1.40 (m, 7H), 1.05 (s, 6H). |
| 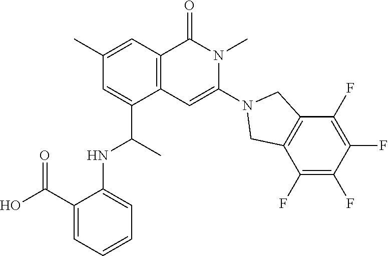 [SMILES: Cc1cc(C(C)Nc2ccccc2C(=O)O)c2cc(N3Cc4c(F)c(F)c(F)c(F)c4C3)n(C)c(=O)c2c1] C₂₈H₂₃F₄N₃O₃ | 89 | | ES+ 526.2 [M + H] | ¹H NMR (400 MHz, DMSO-d₆) δ 12.73 (s, 1H), 8.36 (d, J = 6.0 Hz, 1H), 7.89 (d, J = 1.5 Hz, 1H), 7.82- 7.78 (m, 1H), 7.41 (d, J = 1.9 Hz, 1H), 7.22-7.18 (m, 1H), 6.77 (s, 1H), 6.55- 6.50 (m, 1H), 6.40- 6.34 (m, 1H), 5.29- 5.22 (m, 1H), 4.76 (m, 4H), 3.57 (s, 3H), 2.32 (s, 3H), 1.51 (d, J = 6.5 Hz, 3H). |
| 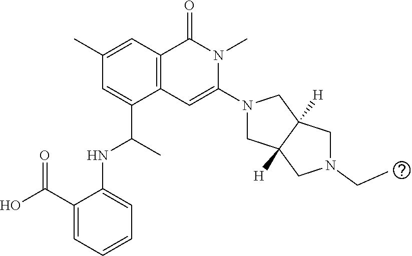 C₂₈H₃₁F₃N₄O₃ | 90 | mixture of trans hexahydro- pyrrolopyrrolyl diastereomers 3 and 4 | ES+ 529.3 [M + H] | ¹H NMR (300 MHz, DMSO-d₆) δ 12.85 (s, 1H), 8.49 (s, 1H), 7.82-7.60 (m, 2H), 7.36-7.30 (m, 1H), 7.24-7.13 (m, 1H), 6.52-6.48 (m, 1H), 6.35 (d, J = 8.5 Hz, 1H), 6.18 (d, J = 2.4 Hz, 1H), 5.11-5.07 (m, 1H), 3.48-3.31 (m, 6H), 3.16 (s, 2H), 2.99 (m, 2H), 2.80-2.70 (m, 2H), 2.37-2.25 (m, 6H), 1.53-1.50 (m, 3H). |
| 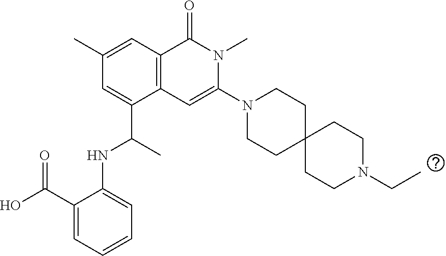 C₃₁H₃₇F₃N₄O₃ | 91 | | ES+ 571.7 [M + H] | ¹H NMR (400 MHz, DMSO-d₆) δ 9.00 (s, 1H), 7.84-7.80 (m, 2H), 7.42 (d, J = 1.9 Hz, 1H), 7.08-7.05 (m, 1H), 6.46-6.43 (m, 1H), 6.38 (s, 1H), 6.28 (d, J = 8.4 Hz, 1H), 5.11-5.08 (m, 1H), 3.47 (s, 3H), 3.17-3.12 (m, 2H), 3.01-2.70 (m, 4H), 2.65-2.59 (m, 4H), 2.31 (s, 3H), 1.81-1.42 (m, 11H). |
| 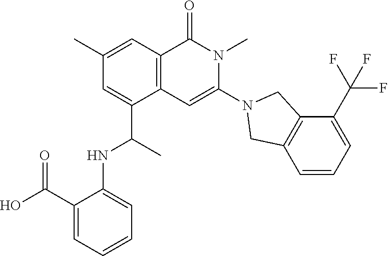 [SMILES: Cc1cc(C(C)Nc2ccccc2C(=O)O)c2cc(N3Cc4cccc(C(F)(F)F)c4C3)n(C)c(=O)c2c1] C₂₉H₂₆F₃N₃O₃ | 92 | enantiomer 1 | ES+ 522.1 [M + H] | ¹H NMR (400 MHz, Methanol-d₄) δ 7.89 (d, J = 1.2 Hz, 1H), 7.82-7.76 (m, 1H), 7.53-7.48 (m, 2H), 7.46-7.36 (m, 2H), 7.05-7.01 (m, 1H), 6.45-6.37 (m, 1H), 6.28 (d, J = 8.4 Hz, 1H), 5.05-5.02 (m, 1H), 4.62 (m, 1H), 4.54-4.47 (m, 4H), 3.61 (s, 3H), 2.28 (s, 3H), 1.52 (d, J = 6.7 Hz, 3H). |
| 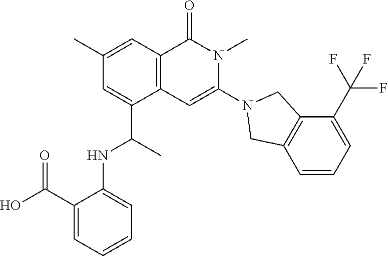 [SMILES: Cc1cc(C(C)Nc2ccccc2C(=O)O)c2cc(N3Cc4cccc(C(F)(F)F)c4C3)n(C)c(=O)c2c1] C₂₉H₂₆F₃N₃O₃ | 93 | enantiomer 2 | ES+ 522.1 [M + H] | ¹H NMR (400 MHz, Methanol-d₄) δ 7.89 (s, 1H), 7.80-7.76 (m, 1H), 7.55-7.47 (m, 2H), 7.46-7.32 (m, 2H), 7.06-7.01 (m, 1H), 6.45-6.37 (m, 1H), 6.27 (d, J = 8.4 Hz, 1H), 5.03- 5.01 (m, 1H), 4.62 (m, 1H), 4.51-4.46 (m, 4H), 3.61 (s, 3H), 2.28 (s, 3H), 1.52 (d, J = 6.7 Hz, 3H) |
| 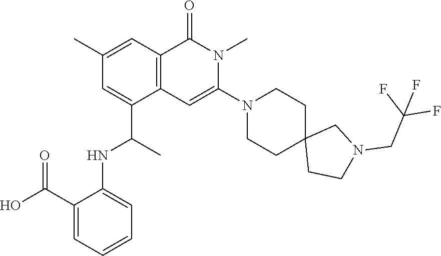 [SMILES: Cc1cc(C(C)Nc2ccccc2C(=O)O)c2cc(N3CCC4(CCN(CC(F)(F)F)C4)CC3)n(C)c(=O)c2c1] C₃₀H₃₅F₃N₄O₃ | 94 | | ES+ 557.5 [M + H] | ¹H NMR (300 MHz, DMSO-d₆) δ 12.73 (s, 1H), 8.37 (d, J = 5.9 Hz, 1H), 7.87 (d, J = 1.5 Hz, 1H), 7.82- 7.77 (m, 1H), 7.40 (d, J = 1.9 Hz, 1H), 7.22-7.18 (m, 1H), 6.58-6.46 (m, 1H), 6.43-6.30 (m, 2H), 5.19-5.10 (m, 1H), 3.48 (s, 3H), 3.26 (d, J = 10.1 Hz, 3H), 3.10-2.90 (m, 2H), 2.79-2.68 (m, 2H), 2.63 (s, 3H), 2.32 (s, 3H), 1.67 (s, 6H), 1.54 (d, J = 6.5 Hz, 3H). |
| 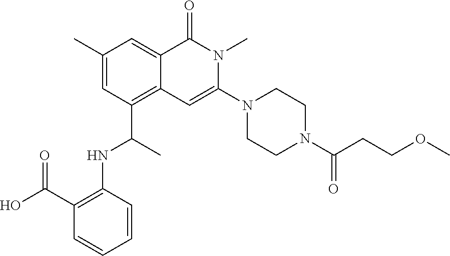 [SMILES: COCCC(=O)N1CCN(c2cc3c(C(C)Nc4ccccc4C(=O)O)cc(C)cc3c(=O)n2C)CC1] C₂₈H₃₄N₄O₅ | 95 | | ES+ 507.6 [M + H] | ¹H NMR (300 MHz, DMSO-d₆) δ 8.42 (s, 1H), 7.88 (d, J = 1.7 Hz, 1H), 7.81-7.79 (m, 1H), 7.42 (d, J = 1.9 Hz, 1H), 7.19- 7.17 (m, 1H), 6.52- 6.50 (m, 1H), 6.43- 6.31 (m, 2H), 5.20- 5.10 (m, 1H), 3.63- 3.51 (m, 6H), 3.34 (m, 7H), 3.24 (s, 3H), 2.65-2.62 (m, 2H), 2.32 (s, 3H), 1.53 (d, J = 6.5 Hz, 3H). |
| 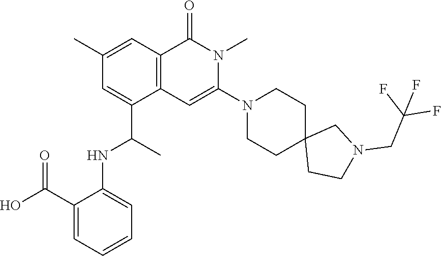 [SMILES: Cc1cc(C(C)Nc2ccccc2C(=O)O)c2cc(N3CCC4(CCN(CC(F)(F)F)C4)CC3)n(C)c(=O)c2c1] C₃₀H₃₅F₃N₄O₃ | 96 | enantiomer 1 | ES+ 557.2 [M + H] | ¹H NMR (300 MHz, DMSO-d₆) δ 7.88- 7.77 (m, 2H), 7.50- 7.40 (m, 1H), 7.07 (s, 1H), 6.50-6.42 (m, 1H), 6.36-6.25 (m, 2H), 5.12-5.08 (m, 1H), 3.48 (s, 3H), 3.23-3.20 (m, 3H), 3.12-2.89 (m, 2H), 2.82-2.65 (m, 3H), 2.63 (s, 2H), 2.31 (s, 3H), 1.67 (s, 6H), 1.51 (d, J = 6.5 Hz, 3H). |
| 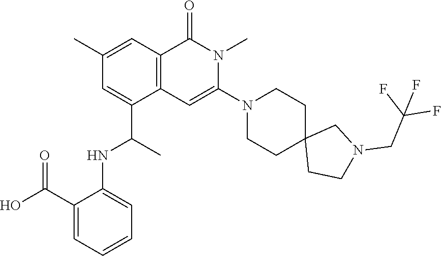 [SMILES: Cc1cc(C(C)Nc2ccccc2C(=O)O)c2cc(N3CCC4(CCN(CC(F)(F)F)C4)CC3)n(C)c(=O)c2c1] C₃₀H₃₅F₃N₄O₃ | 97 | enantiomer 2 | ES+ 557.2 [M + H] | ¹H NMR (300 MHz, DMSO-d₆) δ 8.98 (s, 1H), 7.85-7.79 (m, 2H), 7.48-7.42 (m, 1H), 7.05 (s, 1H), 6.52-6.38 (m, 1H), 6.36-6.24 (m, 2H), 5.11-5.08 (m, 1H), 3.48 (s, 3H), 3.28- 3.24 (m, 3H), 3.11- 2.89 (m, 2H), 2.81- 2.71 (m, 3H), 2.65 (s, 2H), 2.31 (s, 3H), 1.67 (s, 6H), 1.51 (d, J = 6.5 Hz, 3H). |
| 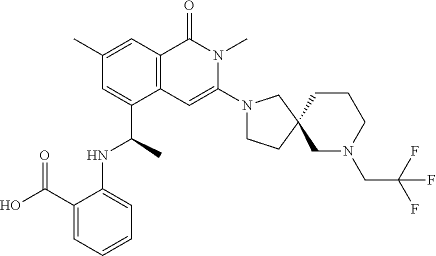 [SMILES: Cc1cc([C@@H](C)Nc2ccccc2C(=O)O)c2cc(N3CC[C@@]4(CCCN(CC(F)(F)F)C4)C3)n(C)c(=O)c2c1] C₃₀H₃₅F₃N₄O₃ | 98 | | ES+ 557.2 [M + H] | ¹H NMR (DMSO-d₆, 400 MHz): δ 12.66 (s, 1H), 8.40 (s, 1H), 7.87-7.77 (m, 2H), 7.39 (d, J = 1.9 Hz, 1H), 7.23-7.17 (m, 1H), 6.55-6.50 (m, 1H), 6.38 (d, J = 8.5 Hz, 1H), 6.26 (s, 1H), 5.14-5.07 (m, 1H), 3.47 (s, 3H), 3.42-3.04 (m, 6H), 2.82 (d, J = 9.5 Hz, 1H), 2.68-2.43 (m, 3H), 2.31 (s, 3H), 1.88-1.76 (m, 1H), 1.70-1.63 (m, 1H), 1.61-1.47 (m, 7H). |
| 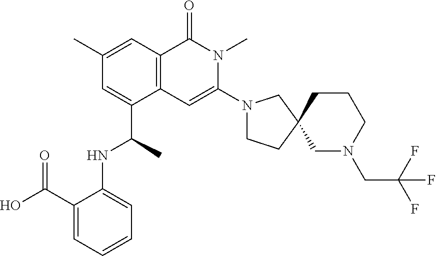 [SMILES: Cc1cc([C@@H](C)Nc2ccccc2C(=O)O)c2cc(N3CC[C@]4(CCCN(CC(F)(F)F)C4)C3)n(C)c(=O)c2c1] C₃₀H₃₅F₃N₄O₃ | 99 | | ES+ 557.2 [M + H] | ¹H NMR (DMSO-d₆, 400 MHz): δ 12.79 (s, 1H), 8.44 (s, 1H), 7.87-7.77 (m, 2H), 7.39 (d, J = 1.9 Hz, 1H), 7.20-7.13 (m, 1H), 6.54-6.51 (m, 1H), 6.38 (d, J = 8.5 Hz, 1H), 6.26 (s, 1H), 5.14-5.07 (m, 1H), 3.47 (s, 3H), 3.42-3.04 (m, 6H), 2.84 (d, J = 9.5 Hz, 1H), 2.66-2.51 (m, 3H), 2.31 (s, 3H), 1.89-1.78 (m, 1H), 1.72-1.62 (m, 1H), 1.59-1.46 (m, 7H). |
| 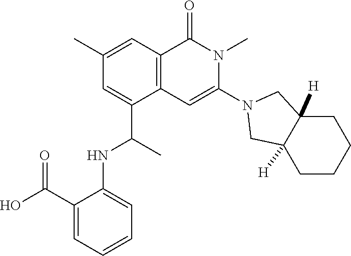 [SMILES: Cc1cc(C(C)Nc2ccccc2C(=O)O)c2cc(N3C[C@@H]4CCCC[C@H]4C3)n(C)c(=O)c2c1] C₂₈H₃₃N₃O₃ | 100 | trans- octahydroisoindolyl diastereomer 1 | ES+ 460.2 [M + H] | ¹H NMR (300 MHz, Methanol-d₄) δ 7.99- 7.78 (m, 2H), 7.48 (d, J = 1.8 Hz, 1H), 7.21-7.09 (m, 1H), 6.59-6.47 (m, 1H), 6.35 (d, J = 8.5 Hz, 1H), 5.09-4.97 (m, 1H), 3.62-3.51 (m, 3H), 3.24-3.03 (m, 4H), 2.35 (s, 3H), 2.00-1.91 (m, 2H), 1.89-1.83 (m, 2H), 1.71-1.60 (m, 5H), 1.45-1.34 (m, 2H), 1.25-1.17 (m, 2H). |
| 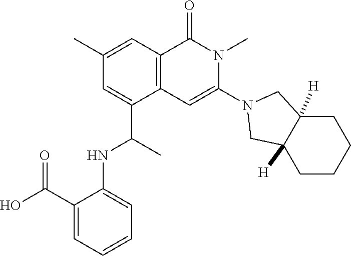 [SMILES: Cc1cc(C(C)Nc2ccccc2C(=O)O)c2cc(N3C[C@H]4CCCC[C@@H]4C3)n(C)c(=O)c2c1] C₂₈H₃₃N₃O₃ | 101 | trans- octahydroisoindolyl diastereomer 2 | ES+ 460.2 [M + H] | ¹H NMR (300 MHz, Methanol-d₄) δ 7.98- 7.81 (m, 2H), 7.48 (d, J = 1.8 Hz, 1H), 7.23-7.07 (m, 1H), 6.59-6.28 (m, 2H), 5.12-4.97 (m, 1H), 3.57 (s, 3H), 3.20- 3.09 (m, 4H), 2.35 (s, 3H), 2.00-1.90 (m, 2H), 1.89-1.81 (m, 2H), 1.70-1.55 (m, 5H), 1.43-1.35 (m, 2H), 1.33-1.27 (m, 2H). |
| 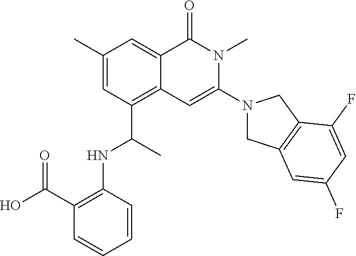 [SMILES: Cc1cc(C(C)Nc2ccccc2C(=O)O)c2cc(N3Cc4cc(F)cc(F)c4C3)n(C)c(=O)c2c1] C₂₈H₂₅F₂N₃O₃ | 102 | enantiomer 1 | ES+ 490.0 [M + H] | ¹H NMR (300 MHz, DMSO-d₆) δ 12.72 (s, 1H), 8.40 (d, J = 5.8 Hz, 1H), 7.89 (d, J = 1.9 Hz, 1H), 7.85- 7.76 (m, 1H), 7.41 (d, J = 1.9 Hz, 1H), 7.27-7.16 (m, 1H), 7.22-7.11 (m, 2H), 6.74 (s, 1H), 6.58- 6.47 (m, 1H), 6.37 (d, J = 8.5 Hz, 1H), 5.29-5.18 (m, 1H), 4.74-4.56 (m, 4H), 3.34 (s, 3H), 2.33 (s, 3H), 1.52 (d, J = 6.4 Hz, 3H). |
| 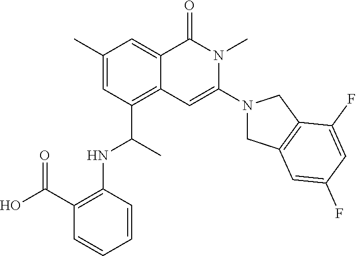 [SMILES: Cc1cc(C(C)Nc2ccccc2C(=O)O)c2cc(N3Cc4cc(F)cc(F)c4C3)n(C)c(=O)c2c1] C₂₈H₂₅F₂N₃O₃ | 103 | enantiomer 2 | ES+ 490.0 [M + H] | ¹H NMR (300 MHz, DMSO-d₆) δ 12.73 (s, 1H), 8.38 (d, J = 6.0 Hz, 1H), 7.89 (d, J = 1.9 Hz, 1H), 7.85- 7.76 (m, 1H), 7.41 (d, J = 1.9 Hz, 1H), 7.27-7.16 (m, 1H), 7.22-7.11 (m, 2H), 6.74 (s, 1H), 6.58- 6.47 (m, 1H), 6.38 (d, J = 8.4 Hz, 1H), 5.29-5.19 (m, 1H), 4.65 (d, J = 2.8 Hz, 4H), 3.34 (s, 3H), 2.33 (s, 3H), 1.52 (d, J = 6.3 Hz, 3H). |
| 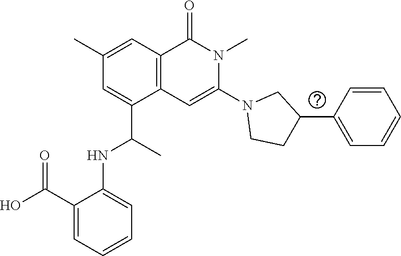 [SMILES: Cc1cc(C(C)Nc2ccccc2C(=O)O)c2cc(N3CCC(c4ccccc4)C3)n(C)c(=O)c2c1] C₃₀H₃₁N₃O₃ | 104 | enantiomer 1 | ES+ 482.2 [M + H] | ¹H NMR (300 MHz, DMSO-d₆) δ 8.47 (s, 1H), 7.87-7.76 (m, 2H), 7.43-7.30 (m, 5H), 7.30-7.19 (m, 1H), 7.19-7.05 (M, 1H), 6.56-6.45 (m, 1H), 6.36 (d, J = 8.5 Hz, 1H), 6.30 (s, 1H), 5.14 (m, 1H), 3.57-3.42 (m, 2H), 3.51 (m, 4H), 3.40- 3.26 (m, 1H), 2.40- 2.30 (m, 2H), 2.30 (s, 3H), 2.14-1.96 (m, 1H), 1.52 (d, J = 6.5 Hz, 3H). |
| 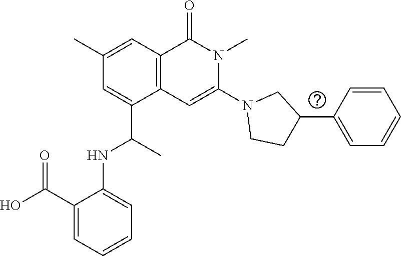 [SMILES: Cc1cc(C(C)Nc2ccccc2C(=O)O)c2cc(N3CCC(c4ccccc4)C3)n(C)c(=O)c2c1] C₃₀H₃₁N₃O₃ | 105 | enantiomer 2 | ES+ 482.2 [M + H] | ¹H NMR (300 MHz, DMSO-d₆) δ 8.47 (s, 1H), 7.87-7.76 (m, 2H), 7.43-7.30 (m, 5H), 7.30-7.19 (m, 1H), 7.19-7.05 (m, 1H), 6.56-6.45 (m, 1H), 6.36 (d, J = 8.5 Hz, 1H), 6.30 (s, 1H), 5.14 (m, 1H), 3.57-3.42 (m, 2H), 3.51 (m, 4H), 3.40- 3.26 (m, 1H), 2.40- 2.30 (m, 2H), 2.30 (s, 3H), 2.14-1.96 (m, 1H), 1.52 (d, J = 6.5 Hz, 3H). |
| 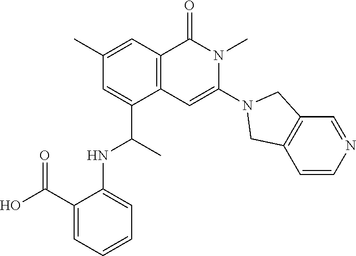 [SMILES: Cc1cc(C(C)Nc2ccccc2C(=O)O)c2cc(N3Cc4ccncc4C3)n(C)c(=O)c2c1] C₂₇H₂₆N₄O₃ | 106 | enantiomer 1 | ES− 453.1 [M − H] | ¹H NMR (300 MHz, DMSO-d₆) δ 8.72- 8.52 (m, 3H), 7.88 (s, 1H), 7.80 (d, J = 7.9 Hz, 1H), 7.52 (s, 1H), 7.44 (d, J = 9.0 Hz, 1H), 7.12-7.10 (m, 1H), 6.72 (d, J = 6.6 Hz, 1H), 6.50- 6.48 (m, 1H), 6.32 (d, J = 8.5 Hz, 1H), 5.18 (m, 1H), 4.65 (d, J = 5.6 Hz, 4H), 3.57 (s, 3H), 2.32 (s, 3H), 1.50 (d, J = 6.5 Hz, 3H). |
| 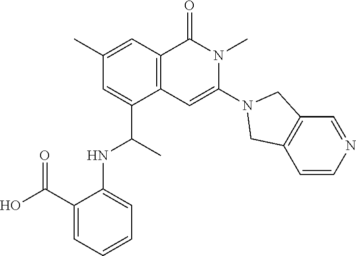 [SMILES: Cc1cc(C(C)Nc2ccccc2C(=O)O)c2cc(N3Cc4ccncc4C3)n(C)c(=O)c2c1] C₂₇H₂₆N₄O₃ | 107 | enantiomer 2 | ES+ 455.1 [M + H] | ¹H NMR (300 MHz, DMSO-d₆) δ 8.72- 8.52 (m, 3H), 7.81 (s, 1H), 7.80 (d, J = 7.9 Hz, 1H), 7.52 (s, 1H), 7.44 (d, J = 9.0 Hz, 1H), 7.12-7.10 (m, 1H), 6.72 (d, J = 6.6 Hz, 1H), 6.50- 6.48 (m, 1H), 6.32 (d, J = 8.5 Hz, 1H), 5.18 (m, 1H), 4.65 (d, J = 5.6 Hz, 4H), 3.57 (s, 3H), 2.32 (s, 3H), 1.50 (d, J = 6.5 Hz, 3H). |
| 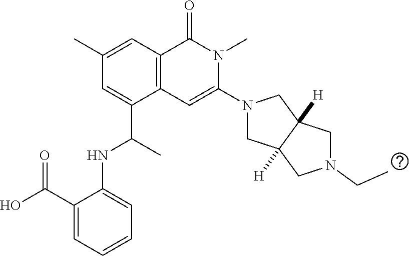 [SMILES: CCN1C[C@H]2CN(c3cc4c(C(C)Nc5ccccc5C(=O)O)cc(C)cc4c(=O)n3C)C[C@@H]2C1] C₂₈H₃₁F₃N₄O₃ | 108 | trans hexahydro- pyrrolopyrrolyl diastereomer 1 | ES− 527.0 [M − H] | ¹H NMR (300 MHz, DMSO-d₆) δ 12.73 (s, 1H), 8.49 (s, 1H), 7.84-7.74 (m, 2H), 7.33 (d, J = 1.9 Hz, 1H), 7.28-7.15 (m, 1H), 6.71-6.32 (m, 1H), 6.35 (d, J = 8.5 Hz, 1H), 6.18 (s, 1H), 5.12-5.05 (m, 1H), 4.03-3.31 (m, 2H), 3.45 (s, 3H), 3.20-3.13 (m, 2H), 3.15 (s, 2H), 2.99 (m, 2H), 2.84-2.72 (m, 2H), 2.34 (m, 2H), 2.30 (s, 3H), 1.54 (d, J = 6.5 Hz, 3H). |
| 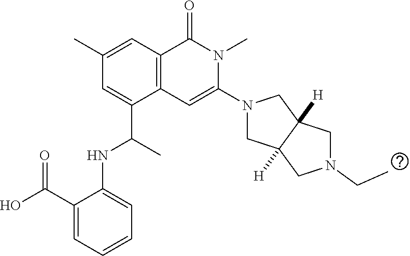 [SMILES: CCN1C[C@H]2CN(c3cc4c(C(C)Nc5ccccc5C(=O)O)cc(C)cc4c(=O)n3C)C[C@@H]2C1] C₂₈H₃₁F₃N₄O₃ | 109 | trans hexahydro- pyrrolopyrrolyl diastereomer 2 | ES− 526.9 [M − H] | ¹H NMR (300 MHz, DMSO-d₆) δ 12.75 (s, 1H), 8.46 (s, 1H), 7.84-7.74 (m, 2H), 7.38 (d, J = 1.9 Hz, 1H), 7.22-7.10 (m, 1H),6.63-6.39 (m, 1H), 6.36 (d, J = 8.4 Hz, 1H), 6.19 (s, 1H), 5.12-5.05 (m, 1H), 4.03-3.31 (m, 2H), 3.45 (s, 3H), 3.20-3.13 (m, 2H), 3.15 (m, 2H), 2.99 (m, 2H), 2.84-2.72 (m, 2H), 2.34 (s, 2H), 2.30 (s, 3H), 1.54 (d, J = 6.5 Hz, 3H). |
| 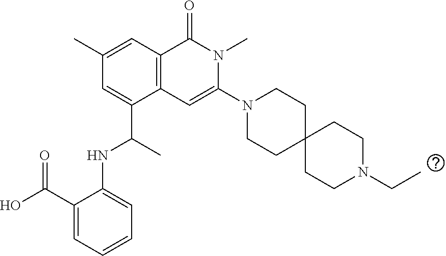 C₃₁H₃₇F₃N₄O₃ | 110 | enantiomer 1 | ES+ 571.1 [M + H] | ¹H NMR (400 MHz, DMSO-d₆) δ 8.41 (s, 1H), 7.86 (s, 1H), 7.80 (d, J = 7.9 Hz, 1H), 7.40 (d, J = 1.8 Hz, 1H), 7.25-7.19 (m, 1H), 6.60-6.48 (m, 1H), 6.38 (d, J = 9.8 Hz, 2H), 5.20- 5.10 (m, 1H), 3.47 (s, 3H), 3.20-3.10 (m, 2H), 2.90-2.89 (m, 4H), 2.70-2.59 (m, 4H), 2.32 (s, 3H), 1.81-1.31 (m, 11H). |
| 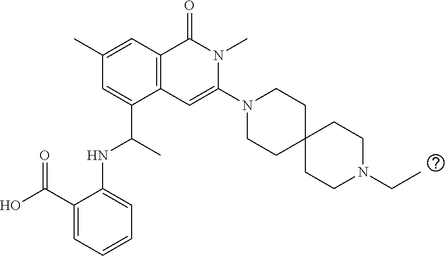 C₃₁H₃₇F₃N₄O₃ | 111 | enantiomer 2 | ES+ 571.1 [M + H] | ¹H NMR (400 MHz, DMSO-d₆) δ 8.41 (s, 1H), 7.86 (s, 1H), 7.80 (d, J = 7.9 Hz, 1H), 7.40 (d, J = 1.8 Hz, 1H), 7.25-7.19 (m, 1H), 6.60-6.48 (m, 1H), 6.38 (d, J = 9.8 Hz, 2H), 5.20- 5.10 (m, 1H), 3.47 (s, 3H), 3.20-3.10 (m, 2H), 2.91-2.89 (m, 4H), 2.70-2.59 (m, 4H), 2.32 (s, 3H), 1.80-1.31 (m, 11H). |
| 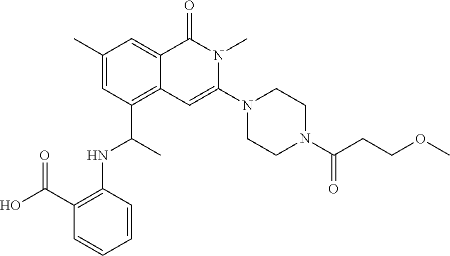 [SMILES: COCCC(=O)N1CCN(c2cc3c(C(C)Nc4ccccc4C(=O)O)cc(C)cc3c(=O)n2C)CC1] C₂₈H₃₄N₄O₅ | 112 | enantiomer 1 | ES+ 507.6 [M + H] | ¹H NMR (300 MHz, DMSO-d₆) δ 8.42 (s, 1H), 7.88 (d, J = 1.7 Hz, 1H), 7.81-7.79 (m, 1H), 7.42 (d, J = 1.9 Hz, 1H), 7.19- 7.17 (m, 1H), 6.52- 6.50 (m, 1H), 6.43- 6.31 (m, 2H), 5.20- 5.10 (m, 1H), 3.63- 3.52 (m, 5H), 3.28- 3.26 (m, 2H), 3.24 (s, 3H), 3.12-2.81 (m, 6H), 2.62-2.65 (m, 2H), 2.32 (s, 3H), 1.53 (d, J = 6.5 Hz, 3H). |
| 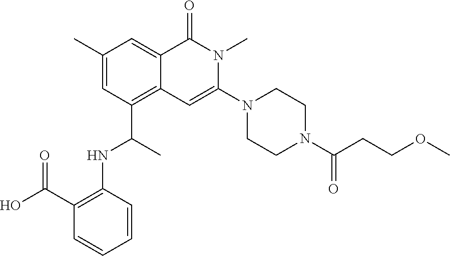 [SMILES: COCCC(=O)N1CCN(c2cc3c(C(C)Nc4ccccc4C(=O)O)cc(C)cc3c(=O)n2C)CC1] C₂₈H₃₄N₄O₅ | 113 | enantiomer 2 | ES+ 507.6 [M + H] | ¹H NMR (300 MHz, DMSO-d₆) δ 8.42 (s, 1H), 7.88 (d, J = 1.7 Hz, 1H), 7.81-7.79 (m, 1H), 7.42 (d, J = 1.9 Hz, 1H), 7.19- 7.17 (m, 1H), 6.52- 6.50 (m, 1H), 6.43- 6.31 (m, 2H), 5.20- 5.10 (m, 1H), 3.63- 3.51 (m, 5H), 3.26- 3.25 (m, 2H), 3.24 (s, 3H), 3.12-2.79 (m, 6H), 2.65-2.62 (m, 2H), 2.32 (s, 3H), 1.53 (d, J = 6.5 Hz, 3H). |
| 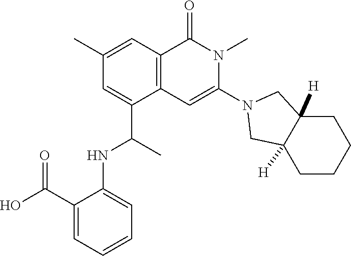 [SMILES: Cc1cc(C(C)Nc2ccccc2C(=O)O)c2cc(N3C[C@@H]4CCCC[C@H]4C3)n(C)c(=O)c2c1] C₂₈H₃₃N₃O₃ | 114 | trans- octahydro- isoindolyl diastereomer 3 | ES+ 460.2 [M + H] | ¹H NMR (400 MHz, DMSO-d₆) δ 8.47 (s, 1H), 7.85-7.75 (m, 2H), 7.32 (d, J = 2.0 Hz, 1H), 7.25-7.09 (m, 1H), 6.59-6.47 (m, 1H), 6.35 (d, J = 8.5 Hz, 1H), 6.10 (s, 1H), 5.09-5.05 (m, 1H), 3.44-3.20 (m, 4H), 3.15 (d, J = 7.2 Hz, 3H), 2.38 (s, 3H), 1.92-1.87 (m, 2H), 1.85-1.72 (m, 3H), 1.60-1.47 (m, 6H), 1.32-1.10 (m, 2H). |
| 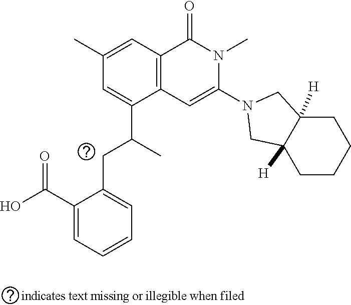 [SMILES: Cc1cc(C(C)Cc2ccccc2C(=O)O)c2cc(N3C[C@H]4CCCC[C@@H]4C3)n(C)c(=O)c2c1] C₂₈H₃₃N₃O₃ | 115 | trans- octahydro- isoindolyl diastereomer 4 | ES+ 460.2 [M + H] | ¹H NMR (400 MHz, DMSO-d₆) δ 8.49 (s, 1H), 7.85-7.77 (m, 2H), 7.32 (d, J = 2.0 Hz, 1H), 7.23-7.13 (m, 1H), 6.63-6.49 (m, 1H), 6.35 (d, J = 8.5 Hz, 1H), 6.10 (s, 1H), 5.09-5.05 (m, 1H), 3.44-3.20 (m, 4H), 3.15 (d, J = 7.5 Hz, 3H), 2.35 (s, 3H), 1.92-1.87 (m, 2H), 1.85-1.72 (m, 3H), 1.60-1.48 (m, 6H), 1.1.03 (m, 2H). |
| 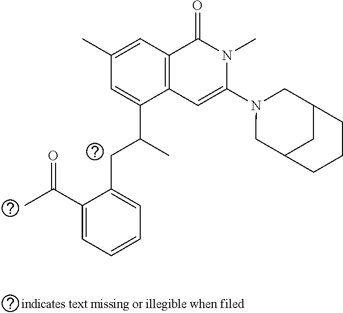 C₂₈H₃₃N₃O₃ | 116 | | ES+ 460.6 [M + H] | ¹H NMR (300 MHz, Methanol-d₄) δ 7.99- 7.93 (m, 1H), 7.90- 7.88 (m, 1H), 7.53 (d, J = 1.9 Hz, 1H), 7.15-7.12 (m, 1H), 6.57-6.46 (m, 2H), 6.37 (d, J = 8.5 Hz, 1H), 5.12-5.08 (m, 1H), 4.60 (m, 4H), 3.69 (s, 3H), 3.22- 3.15 (m, 1H), 3.08- 2.91 (m, 2H), 2.72- 2.53 (m, 1H), 2.37 (s, 3H), 2.00-1.97 (m, 2H), 1.78-1.72 (m, 2H), 1.65 (d, J = 6.5 Hz, 3H). |
| 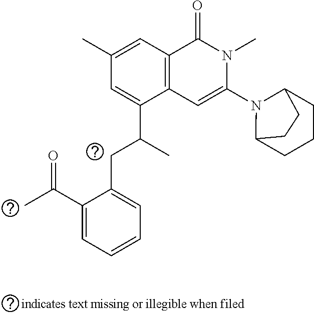 C₂₇H₃₁N₃O₃ | 117 | enantiomer 1 | ES+ 446.1 [M + H] | ¹H NMR (300 MHz, DMSO-d₆) δ 8.56 (s, 1H), 7.86-7.76 (m, 2H), 7.41 (d, J = 1.9 Hz, 1H), 7.21-7.10 (m, 1H), 6.82-6.44 (m, 1H), 6.51-6.33 (m, 1H), 6.12 (s, 1H), 5.10-5.03 (m, 1H), 3.82-3.61 (m, 2H), 3.56 (s, 3H), 2.32 (s, 3H), 2.02- 1.91 (m, 2H), 1.90- 1.73 (m, 2H), 1.73- 1.56 (m, 3H), 1.61- 1.47 (m, 6H). |
| 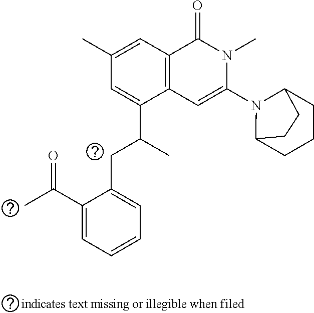 C₂₇H₃₁N₃O₃ | 118 | enantiomer 2 | ES+ 446.1 [M + H] | ¹H NMR (300 MHz, DMSO-d₆) δ 8.39 (s, 1H), 7.87-7.76 (m, 2H), 7.41 (d, J = 1.9 Hz, 1H), 7.25-7.13 (m, 1H), 6.52-6.12 (m, 1H), 6.49-6.40 (m, 1H), 6.12 (s, 1H), 5.10-5.03 (m, 1H), 3.82-3.61 (m, 2H), 3.56 (s, 3H), 2.32 (s, 3H), 2.02- 1.91 (m, 2H), 1.90- 1.73 (m, 2H), 1.73- 1.56 (m, 3H), 1.61- 1.47 (m, 6H). |
| 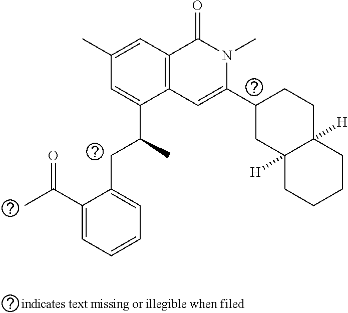 C₂₉H₃₅N₃O₃ | 119 | cis diastereomer 1 | ES+ 474.0 [M + H] | ¹H NMR (Methanol- d4, 400 MHz, ppm): 8 8.01-7.85 (m, 2H), 7.54 (d, J = 1.9 Hz, 1H), 7.20-7.15 (m, 1H), 6.56-6.52 (m, 1H), 6.49-6.38 (m, 2H), 5.12-5.06 (m, 1H), 3.62 (s, 3H), 3.21 (m, 1H), 2.99 (d, J = 11.2 Hz, 1H), 2.82 (m, 1H), 2.62 (m, 1H), 2.37 (s, 3H), 2.18-1.95 (d, J = 6.5 Hz, 2H), 1.93-1.81 (m, 3H), 1.63 (d, J = 6.7 Hz, 4H), 1.55-1.32 (m, 6H). |
| 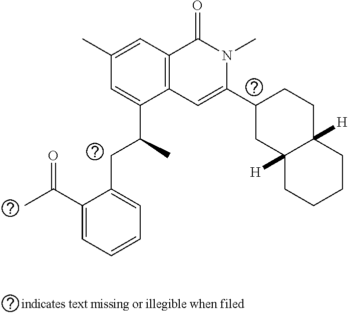 C₂₉H₃₅N₃O₃ | 120 | cis diastereomer 2 | ES+ 474.1 [M + H] | ¹H NMR (Methanol- d4, 300 MHz, ppm): 8 8.01-7.85 (m, 2H), 7.54 (d, J = 1.9 Hz, 1H), 7.20-7.15 (m, 1H), 6.56-6.52 (m, 1H), 6.49-6.38 (m, 2H), 5.12-5.06 (m, 1H), 3.62 (s, 3H), 3.21 (m, 1H), 2.99 (d, J = 11.2 Hz, 1H), 2.82 (m, 1H), 2.62 (m, 1H), 2.37 (s, 3H), 2.18-1.95 (d, J = 6.5 Hz, 2H), 1.93-1.81 (m, 3H), 1.63 (d, J = 6.7 Hz, 4H), 1.55-1.30 (m, 6H). |
| 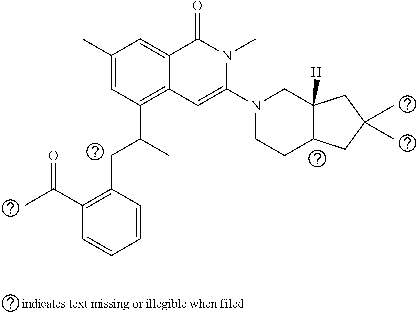 [SMILES: CC(=O)c1ccccc1CC(C)c1cc(C)cc2c(=O)n(C)c(N3CCC4CC(C)(C)C[C@H]4C3)cc12] C₂₇H₃₀F₂N₄O₃ | 121 | diastereomer 1 | ES− 495.0 [M − H] | ¹H NMR (400 MHz, Methanol-d₄) δ 7.98 (s, 1H), 7.91-7.89 (m, 1H), 7.54 (s, 1H), 7.20-7.16 (m, 1H), 6.65-6.50 (m, 2H), 6.45-6.43 (m, 1H), 5.18-5.11 (m, 1H), 3.62 (s, 3H), 3.50-3.38 (m, 1H), 3.32-3.22 (m, 2H), 3.10-3.07 (m, 1H), 3.00-2.91 (m, 1H), 2.75-2.69 (m, 3H), 2.62-2.57 (m, 1H), 2.39 (s, 3H), 2.36- 2.26 (m, 1H), 2.10- 1.91 (m, 1H), 1.65 1.55 (m, 3H). |
| 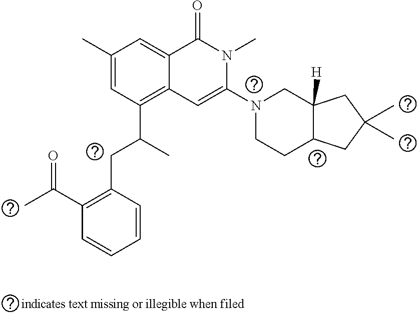 [SMILES: CC(=O)c1ccccc1CC(C)c1cc(C)cc2c(=O)n(C)c(N3CCC4CC(C)(C)C[C@H]4C3)cc12] C₂₇H₃₀F₂N₄O₃ | 122 | diastereomer 2 | ES− 495.1 [M − H] | ¹H NMR (400 MHz, Methanol-d₄) δ 7.98 (s, 1H), 7.92-7.89 (m, 1H), 7.55 (s, 1H), 7.19-7.16 (m, 1H), 6.65-6.50 (m, 2H), 6.44-6.43 (m, 1H), 5.19-5.11 (m, 1H), 3.62 (s, 3H), 3.50-3.38 (m, 1H), 3.32-3.21 (m, 2H), 3.11-3.07 (m, 1H), 2.99-2.91 (m, 1H), 2.77-2.70 (m, 3H), 2.63-2.57 (m, 1H), 2.39 (s, 3H), 2.36- 2.26 (m, 1H), 2.10- 1.91 (m, 1H), 1.66- 1.54 (m, 3H). |
| 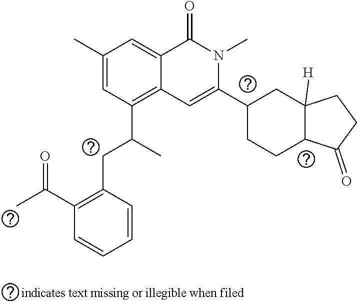 C₂₇H₃₀N₄O₄ | 123 | diastereomer 1 | ES+ 475.1 [M + H] | ¹H NMR (400 MHz, Methanol-d₄) δ 7.98 (d, J = 1.5 Hz, 1H), 7.89 (d, J = 8.0, 1.7 Hz, 1H), 7.55 (d, J = 1.8 Hz, 1H), 7.15- 7.11 (m, 1H), 6.57- 6.48 (m, 2H), 6.38 (d, J = 8.5 Hz, 1H), 5.12-5.05 (m, 1H), 4.05 (d, J = 12.9 Hz, 1H), 3.98-3.87 (m, 1H), 3.66 (s, 3H), 3.58-3.42 (m, 1H), 3.27-3.13 (m, 2H), 2.70-2.64 (m, 1H), 2.57-2.41 (m, 3H), 2.39 (s, 3H), 2.34- 2.20 (m, 1H), 1.83- 1.71 (m, 1H), 1.64 (d, J = 6.7 Hz, 3H). |
| 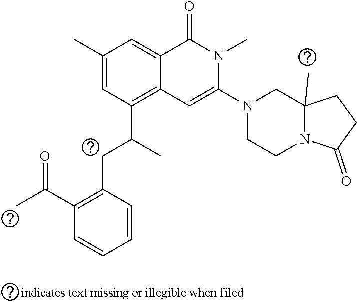 C₂₇H₃₀N₄O₄ | 124 | diastereomer 2 | ES+ 475.1 [M + H] | ¹H NMR (400 MHz, Methanol-d₄) δ 7.99 (d, J = 1.7 Hz, 1H), 7.89 (m, 1H), 7.55 (d, J = 2.0 Hz, 1H), 7.18-7.14 (m, 1H), 6.57-6.49 (m, 2H), 6.43 (d, J = 8.5 Hz, 1H), 5.12-5.05 (m, 1H), 4.06 (d, J = 12.9 Hz, 1H), 3.98- 3.87 (m, 1H), 3.66 (s, 3H), 3.58-3.42 (m, 1H), 3.27-3.13 (m, 2H), 2.70-2.64 (m, 1H), 2.57-2.41 (m, 3H), 2.39 (s, 3H), 2.34-2.20 (m, 1H), 1.83-1.71 (m, 1H), 1.64 (d, J = 6.7 Hz, 3H). |
| 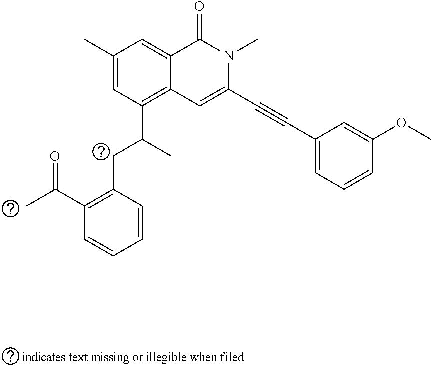 [SMILES: COc1cccc(C#Cc2cc3c(C(C)Cc4ccccc4C(C)=O)cc(C)cc3c(=O)n2C)c1] C₂₉H₂₆N₂O₄ | 125 | enantiomer 1 | ES− 464.9 [M − H] | ¹H NMR (400 MHz, Methanol-d₄) δ 8.07- 8.01 (m, 1H), 7.90 (dd, J = 7.9, 1.7 Hz, 1H), 7.62 (d, J = 1.9 Hz, 1H), 7.49 (s, 1H), 7.39-7.30 (m, 1H), 7.25-7.16 (m, 2H), 7.12-7.07 (m, 1H), 7.05-7.01 (m, 1H), 6.58-6.44 (m, 1H), 6.27 (d, J = 8.5, 1.1 Hz, 1H), 5.26- 5.12 (m, 1H), 3.85 (d, J = 6.1 Hz, 6H), 2.40 (s, 3H), 1.62 (d, J = 6.7 Hz, 3H). |
| 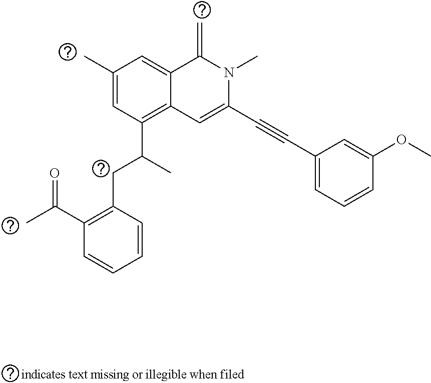 [SMILES: C=C1c2cc(C)cc(C(C)Cc3ccccc3C(C)=O)c2C=C(C#Cc2cccc(OC)c2)N1C] C₂₉H₂₆N₂O₄ | 126 | enantiomer 2 | ES− 464.9 [M − H] | ¹H NMR (400 MHz, Methanol-d₄) δ 8.09- 8.01 (m, 1H), 7.90 (dd, J = 7.9, 1.7 Hz, 1H), 7.60 (d, J = 1.9 Hz, 1H), 7.49 (s, 1H), 7.38-7.30 (m, 1H), 7.26-7.20 (m, 1H), 7.22-7.16 (m, 1H), 7.15-7.08 (m, 1H), 7.05-7.00 (m, 1H), 6.55-6.50 (m, 1H), 6.37-6.21 (m, 1H), 5.23-5.19 (m, 1H), 3.84 (d, J = 5.8 Hz, 6H), 2.40 (s, 3H), 1.62 (d, J = 6.7 Hz, 3H). |
| 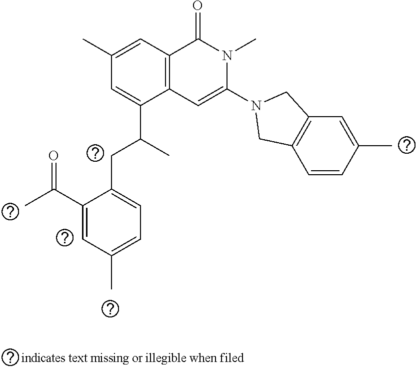 C₂₇H₂₄ClFN₄O₃ | 127 | enantiomer 1 | ES+ 507.2 [M + H] | ¹H NMR (300 MHz, Methanol-d₄) δ 7.98 (s, 1H), 7.55 (s, 1H), 7.39-7.29 (m, 1H), 7.17-6.90 (m, 3H), 6.72 (d, J = 9.1 Hz, 2H), 5.05-5.00 (m, 1H), 4.59 (s, 2H), 4.55 (s, 2H), 3.69 (s, 3H), 2.37 (s, 3H), 1.62 (d, J = 6.6 Hz, 3H). |
| 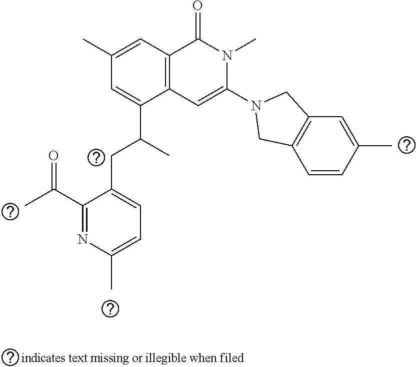 C₂₇H₂₄ClFN₄O₃ | 128 | enantiomer 2 | ES+ 507.2 [M + H] | ¹H NMR (300 MHz, Methanol-d₄) δ 7.98 (s, 1H), 7.56 (s, 1H), 7.39-7.29 (m, 1H), 7.16-6.93 (m, 3H), 6.72 (d, J = 12.2 Hz, 2H), 5.05-5.00 (m, 1H), 4.59 (s, 2H), 4.55 (s, 2H), 3.69 (s, 3H), 2.37 (s, 3H), 1.62 (d, J = 6.7 Hz, 3H). |
| 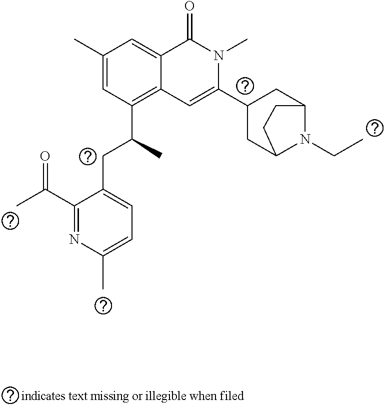 C₂₇H₂₉ClF₃N₅O₃ | 129 | | ES+ 564.0 [M + H] | ¹H NMR (400 MHz, Methanol-d₄) δ 8.01 (m, 1H), 7.50 (d, J = 1.8 Hz, 1H), 7.21 (d, J = 8.9 Hz, 1H), 6.88 (d, J = 9.0 Hz, 1H), 6.59 (s, 1H), 5.24- 5.15 (m, 1H), 3.69 (s, 3H), 3.43 (s, 2H), 3.19-3.05 (m, 4H), 3.05-2.96 (m, 2H), 2.39 (s, 3H), 2.13- 2.00 (m, 4H), 1.67 (d, J = 6.6 Hz, 3H). |
| 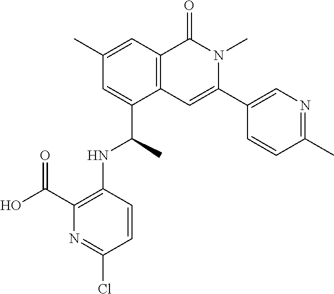 [SMILES: Cc1cc([C@@H](C)Nc2ccc(Cl)nc2C(=O)O)c2cc(-c3ccc(C)nc3)n(C)c(=O)c2c1] C₂₅H₂₃ClN₄O₃ | 130 | | ES+ 463.1 [M + H] | ¹H NMR (300 MHz, Methanol-d₄) δ 8.65- 8.58 (m, 1H), 8.14- 8.07 (m, 1H), 7.97- 7.88 (m, 1H), 7.62- 7.43 (m, 2H), 7.14 (d, J = 8.9 Hz, 1H), 6.96 (s, 1H), 6.84 (d, J = 9.0 Hz, 1H), 5.26- 5.13 (m, 1H), 3.45 (s, 3H), 2.64 (s, 3H), 2.42 (s, 3H), 1.63 (d, J = 6.6 Hz, 3H). |
| 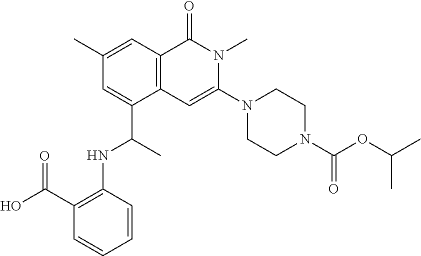 [SMILES: Cc1cc(C(C)Nc2ccccc2C(=O)O)c2cc(N3CCN(C(=O)OC(C)C)CC3)n(C)c(=O)c2c1] C₂₈H₃₄N₄O₅ | 131 | enantiomer 1 | ES− 505.2 [M − H] | ¹H NMR (300 MHz, DMSO-d₆) δ 8.53 (s, 1H), 7.88 (d, J = 1.6 Hz, 1H), 7.80-7.78 (m, 1H), 7.42 (d, J = 1.9 Hz, 1H), 7.20- 7.10 (m, 1H), 6.55- 6.44 (m, 1H), 6.41 (s, 1H), 6.35 (d, J = 8.5 Hz, 1H), 5.20- 5.15 (m, 1H), 4.85- 4.60 (m, 1H), 3.95 (s, 2H), 3.52 (s, 3H), 3.04 (s, 6H), 2.32 (s, 3H), 1.52 (d, J = 6.5 Hz, 3H), 1.22 (d, J = 6.2 Hz, 6H). |
| 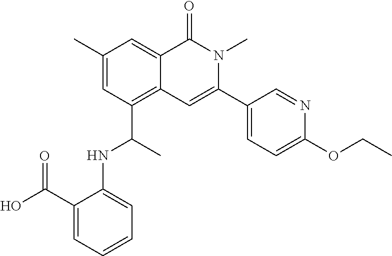 [SMILES: CCOc1ccc(-c2cc3c(C(C)Nc4ccccc4C(=O)O)cc(C)cc3c(=O)n2C)cn1] C₂₇H₂₇N₃O₄ | 132 | enantiomer 1 | ES+ 458.1 [M + H] | ¹H NMR (300 MHz, DMSO-d₆) δ 12.71 (s, 1H), 8.46-8.35 (m, 2H), 8.02-7.91 (m, 2H), 7.85-7.76 (m, 1H), 7.48 (d, J = 1.9 Hz, 1H), 7.22- 7.10 (m, 1H), 6.98- 6.87 (m, 2H), 6.57- 6.46 (m, 1H), 6.34 (d, J = 8.5 Hz, 1H), 5.23-5.18 (m, 1H), 4.45-4.32 (m, 2H), 3.36 (s, 3H), 2.37 (s, 3H), 1.51 (d, J = 6.4 Hz, 3H), 1.42-1.31 (m, 3H). |
| 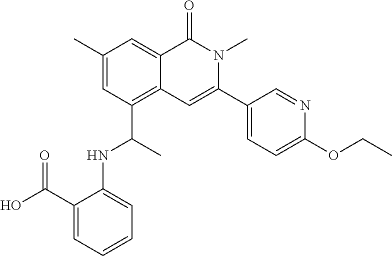 [SMILES: CCOc1ccc(-c2cc3c(C(C)Nc4ccccc4C(=O)O)cc(C)cc3c(=O)n2C)cn1] C₂₇H₂₇N₃O₄ | 133 | enantiomer 2 | ES+ 458.1 [M + H] | ¹H NMR (300 MHz, DMSO-d₆) δ 12.71 (s, 1H), 8.46-8.35 (m, 2H), 8.02-7.91 (m, 2H), 7.85-7.76 (m, 1H), 7.48 (d, J = 1.9 Hz, 1H), 7.22- 7.10 (m, 1H), 6.98- 6.87 (m, 2H), 6.57- 6.46 (m, 1H), 6.34 (d, J = 8.5 Hz, 1H), 5.23-5.18 (m, 1H), 4.45-4.32 (m, 2H), 3.36 (s, 3H), 2.37 (s, 3H), 1.51 (d, J = 6.4 Hz, 3H), 1.42-1.31 (m, 3H). |
| 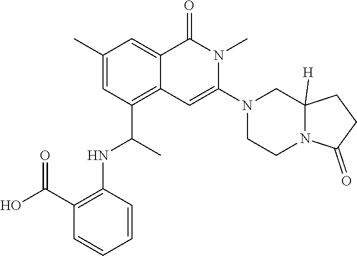 [SMILES: Cc1cc(C(C)Nc2ccccc2C(=O)O)c2cc(N3CCN4C(=O)CCC4C3)n(C)c(=O)c2c1] C₂₇H₃₀N₄O₄ | 134 | diastereomer 3 | ES+ 473.0 [M + H] | ¹H NMR (400 MHz, Methanol-d₄) δ 8.02- 7.99 (m, 1H), 7.91- 7.89 (m, 1H), 7.55 (s, 1H), 7.20-7.10 (m, 1H), 6.58-6.46 (m, 2H), 6.43-6.38 (m, 1H), 5.11-5.09 (m, 1H), 4.06-4.02 (m, 1H), 3.92-3.82 (m, 1H), 3.66 (s, 3H), 3.48 (s, 1H), 3.21-3.19 (m, 1H), 3.10 (s, 1H), 2.78- 2.66 (m, 1H), 2.56- 2.36 (m, 6H), 2.35- 2.22 (m, 1H), 1.76- 1.67 (m, 1H), 1.64- 1.62 (m, 3H). |
| 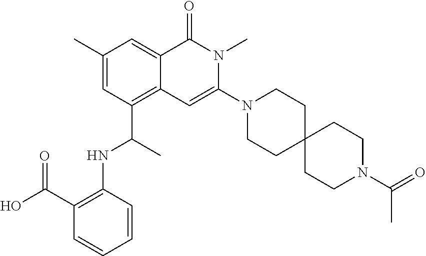 [SMILES: CC(=O)N1CCC2(CC1)CCN(c1cc3c(C(C)Nc4ccccc4C(=O)O)cc(C)cc3c(=O)n1C)CC2] C₃₁H₃₈N₄O₄ | 135 | enantiomer 1 | ES− 529.2 [M − H] | ¹H NMR (400 MHz, Methanol-d₄) δ 7.96 (s, 1H), 7.92-7.89 (m, 1H), 7.58-7.56 (m, 1H), 7.15-7.10 (m, 1H), 6.59-6.43 (m, 2H), 6.42-6.37 (m, 1H), 5.12-5.05 (m, 1H), 3.74-3.60 (m, 5H), 3.60-3.48 (m, 2H), 3.10-2.80 (m, 4H), 2.38 (s, 3H), 2.11 (s, 3H), 1.89-1.87 (m, 2H), 1.75-1.63 (m, 7H), 1.60-1.40 (m, 2H). |
| 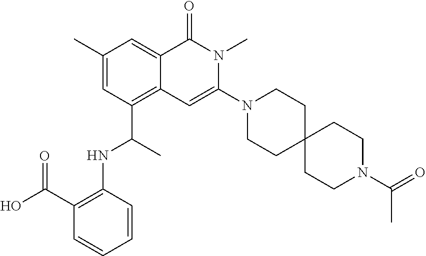 [SMILES: CC(=O)N1CCC2(CC1)CCN(c1cc3c(C(C)Nc4ccccc4C(=O)O)cc(C)cc3c(=O)n1C)CC2] C₃₁H₃₈N₄O₄ | 136 | enantiomer 2 | ES− 529.2 [M − H] | ¹H NMR (400 MHz, Methanol-d₄) δ 7.96 (s, 1H), 7.93-7.89 (m, 1H), 7.59-7.56 (m, 1H), 7.15-7.11 (m, 1H), 6.59-6.44 (m, 2H), 6.42-6.38 (m, 1H), 5.12-5.04 (m, 1H), 3.75-3.60 (m, 5H), 3.60-3.50 (m, 2H), 3.10-2.81 (m, 4H), 2.38 (s, 3H), 2.11 (s, 3H), 1.90-1.87 (m, 2H), 1.75-1.62 (m, 7H), 1.61-1.42 (m, 2H). |
| 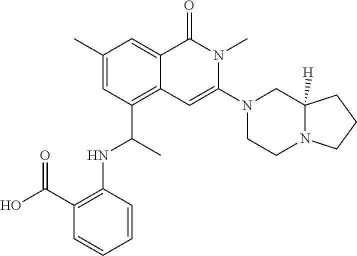 [SMILES: Cc1cc(C(C)Nc2ccccc2C(=O)O)c2cc(N3CCN4CCC[C@@H]4C3)n(C)c(=O)c2c1] C₂₇H₃₂N₄O₃ | 137 | diastereomer 1 | ES+ 461.2 [M + H] | ¹H NMR (400 MHz, Methanol-d₄) δ 7.97 (s, 1H), 7.90-7.87 (m, 1H), 7.58-7.56 (m, 1H), 7.10-7.06 (m, 1H), 6.66 (s, 1H), 6.54-6.30 (m, 2H), 5.07 (s, 1H), 3.61 (s, 3H), 3.37- 3.40 (m, 3H), 3.25 (s, 1H), 3.03 (s, 1H), 2.87 (s, 2H), 2.81- 2.71 (m, 2H), 2.39 (s, 3H), 2.03-1.91 (m, 3H), 1.70-1.61 (m, 4H). |
| 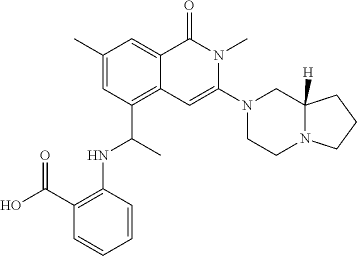 [SMILES: Cc1cc(C(C)Nc2ccccc2C(=O)O)c2cc(N3CCN4CCC[C@H]4C3)n(C)c(=O)c2c1] C₂₇H₃₂N₄O₃ | 138 | diastereomer 2 | ES− 459.0 [M − H] | ¹H NMR (400 MHz, Methanol-d₄) δ 7.97 (s, 1H), 7.93-7.82 (m, 1H), 7.63-7.52 (m, 1H), 7.12-7.08 (m, 1H), 6.68 (s, 1H), 6.51-6.49 (m, 1H), 6.41-6.38 (m, 1H), 5.12-5.07 (m, 1H), 3.61 (s, 3H), 3.43 (s, 1H), 3.2 (s, 2H) 3.02 (s, 1H), 2.74 (s, 3H), 2.56 (s, 1H), 2.39 (s, 3H), 2.12-1.99 (m, 4H), 1.65 (d, J = 6.7 Hz, 4H). |
| 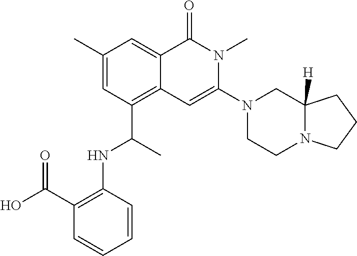 [SMILES: Cc1cc(C(C)Nc2ccccc2C(=O)O)c2cc(N3CCN4CCC[C@H]4C3)n(C)c(=O)c2c1] C₂₇H₃₂N₄O₃ | 139 | diastereomer 3 | ES− 459.0 [M − H] | ¹H NMR (400 MHz, Methanol-d₄) δ 7.97 (s, 1H), 7.92-7.82 (m, 1H), 7.63-7.51 (m, 1H), 7.11-7.08 (m, 1H), 6.68 (s, 1H), 6.51-6.47 (m, 1H), 6.42-6.38 (m, 1H), 5.12-5.08 (m, 1H), 3.61 (s, 3H), 3.43 (s, 1H), 3.20 (s, 2H) 3.02 (s, 1H), 2.74 (s, 3H), 2.56 (s, 1H), 2.39 (s, 3H), 2.12-1.98 (m, 4H), 1.65 (d, J = 6.7 Hz, 4H) |
| 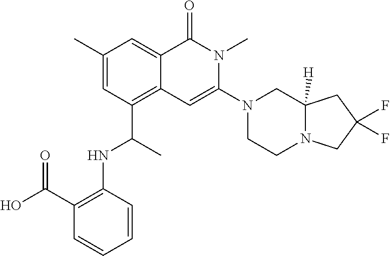 [SMILES: Cc1cc(C(C)Nc2ccccc2C(=O)O)c2cc(N3CCN4CC(F)(F)C[C@@H]4C3)n(C)c(=O)c2c1] C₂₇H₃₀F₂N₄O₃ | 140 | mixture of diastereomers | ES− 495.1 [M − H] | ¹H NMR (400 MHz, Methanol-d₄) δ 7.98 (s, 1H), 7.90-7.88 (m, 1H), 7.53-7.51 (d, J = 1.9 Hz, 1H), 7.16-7.12 (m, 1H), 6.60-6.47 (m, 2H), 6.40-6.38 (d, J = 8.5 Hz, 1H), 5.18- 5.13 (m, 1H), 3.62 (s, 3H), 3.48-3.41 (m, 1H), 3.27 (s, 1H), 3.20-3.18 (d, J = 11.7 Hz, 1H), 3.08-3.05 (d, J = 11.1 Hz, 1H), 2.92 (s, 1H), 2.75-2.66 (m, 3H), 2.61-2.50 (m, 1H), 2.38-2.32 (m, 4H), 2.05-1.98 (m, 1H), 1.64-1.50 (d, J = 6.7 Hz, 3H). |
| 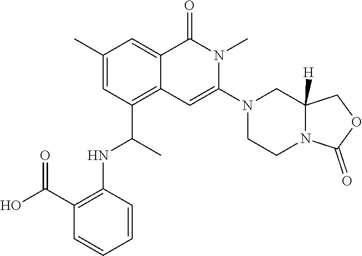 [SMILES: Cc1cc(C(C)Nc2ccccc2C(=O)O)c2cc(N3CCN4C(=O)OC[C@H]4C3)n(C)c(=O)c2c1] C₂₆H₂₈N₄O₅ | 141 | diastereomer 1 | ES− 475.2 [M − H] | ¹H NMR (400 MHz, Methanol-d₄) δ 8.01- 7.98 (m, 1H), 7.91- 7.89 (m, 1H), 7.57- 7.55 (m, 1H), 7.24- 7.10 (m, 1H), 6.57- 6.49 (m, 2H), 6.42- 6.40 (m, 1H), 5.12- 5.08 (m, 1H), 4.63- 4.42 (m, 1H), 4.23- 4.05 (m, 2H), 3.88- 3.74 (m, 1H), 3.65 (s, 3H), 3.31-3.21 (m, 2H), 3.20-3.12 (m, 1H), 2.79-2.65 (m, 2H), 2.39 (s, 3H), 1.64 (d, J = 6.7 Hz, 3H). |
| 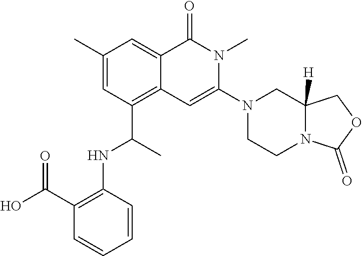 [SMILES: Cc1cc(C(C)Nc2ccccc2C(=O)O)c2cc(N3CCN4C(=O)OC[C@H]4C3)n(C)c(=O)c2c1] C₂₆H₂₈N₄O₅ | 142 | diastereomer 2 | ES− 475.2 [M − H] | ¹H NMR (400 MHz, Methanol-d₄) δ 8.01- 7.98 (m, 1H), 7.89- 7.82 (m, 1H), 7.55- 7.42 (m, 1H), 7.15- 7.08 (m, 1H), 6.57- 6.49 (m, 2H), 6.42- 6.39 (m, 1H), 5.12- 5.06 (m, 1H), 4.58- 4.45 (m, 1H), 4.16- 4.05 (m, 2H), 3.85- 3.80 (m, 1H), 3.65 (s, 3H), 3.41-3.33 (m, 2H), 3.20-3.12 (m, 1H), 2.70-2.66 (m, 2H), 2.39 (s, 3H), 1.64 (d, J = 6.7 Hz, 3H). |
| 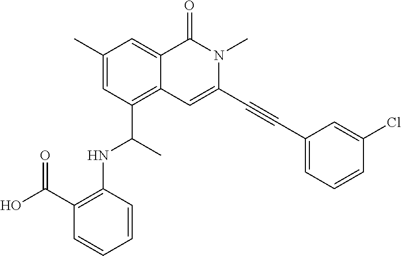 [SMILES: Cc1cc(C(C)Nc2ccccc2C(=O)O)c2cc(C#Cc3cccc(Cl)c3)n(C)c(=O)c2c1] C₂₈H₂₃ClN₂O₃ | 143 | enantiomer 1 | ES+ 471.0 [M + H] | ¹H NMR (400 MHz, Chloroform-d) δ 8.20-8.17 (m, 2H), 8.05-7.96 (m, 1H), 7.59-7.54 (m, 2H), 7.52-7.46 (m, 1H), 7.42-7.38 (m, 1H), 7.22-7.20 (m, 2H), 7.14 (s, 1H), 6.62- 6.58 (m, 1H), 6.26 (d, J = 8.5 Hz, 1H), 5.12-5.08 (m, 1H), 3.84 (s, 3H), 2.41 (s, 3H), 1.67 (d, J = 6.7 Hz, 3H). |
| 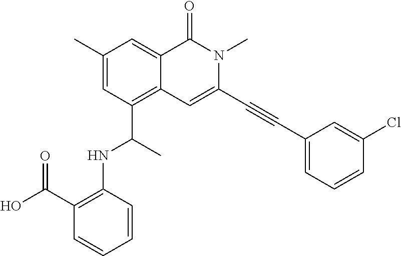 [SMILES: Cc1cc(C(C)Nc2ccccc2C(=O)O)c2cc(C#Cc3cccc(Cl)c3)n(C)c(=O)c2c1] C₂₈H₂₃ClN₂O₃ | 144 | enantiomer 2 | ES+ 471.0 [M + H] | ¹H NMR (400 MHz, Chloroform-d) δ 8.22-8.19 (m, 2H), 8.05-7.96 (m, 1H), 7.59-7.54 (m, 2H), 7.44-7.37 (m, 1H), 7.36-7.32 (m, 1H), 7.22-7.20 (m, 2H), 7.14 (s, 1H), 6.62- 6.58 (m, 1H), 6.26 (d, J = 8.5 Hz, 1H), 5.12-5.08 (m, 1H), 3.82 (s, 3H), 2.43 (s, 3H), 1.67 (d, J = 6.7 Hz, 3H). |
| 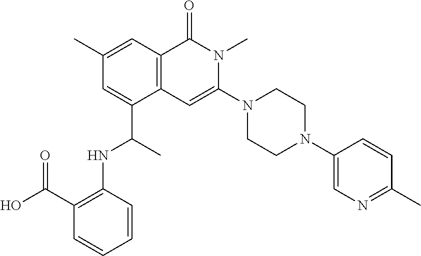 [SMILES: Cc1cc(C(C)Nc2ccccc2C(=O)O)c2cc(N3CCN(c4ccc(C)nc4)CC3)n(C)c(=O)c2c1] C₃₀H₃₃N₅O₃ | 145 | enantiomer 1 | ES+ 512.2 [M + H] | ¹H NMR (400 MHz, Methanol-d₄) δ 8.18 (d, J = 3.0 Hz, 1H), 7.99 (s, 1H), 7.92- 7.88 (m, 1H), 7.68- 7.63 (m, 1H), 7.52- 7.41 (m, 1H), 7.23 (d, J = 8.6 Hz, 1H), 7.19-7.10 (m, 1H), 6.79-6.52 (m, 2H), 6.39 (d, J = 8.5 Hz, 1H), 5.15-5.08 (m, 1H), 3.95-3.61 (m, 5H), 3.48-3.34 (m, 1H), 3.33-3.15 (m, 2H), 3.14-2.97 (m, 3H), 2.51 (s, 3H), 2.39 (s, 3H), 1.65 (d, J = 6.6 Hz, 3H). |
| 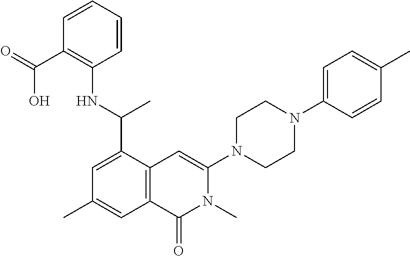 [SMILES: Cc1ccc(N2CCN(c3cc4c(C(C)Nc5ccccc5C(=O)O)cc(C)cc4c(=O)n3C)CC2)cc1] C₃₀H₃₃N₅O₃ | 146 | enantiomer 2 | ES+ 512.2 [M + H] | ¹H NMR (400 MHz, Methanol-d₄) δ 8.15 (d, J = 2.9 Hz, 1H), 7.99 (s, 1H), 7.92- 7.88 (m, 1H), 7.68- 7.63 (m, 1H), 7.52- 7.41 (m, 1H), 7.21 (d, J = 8.6 Hz, 1H), 7.16-7.12 (m, 1H), 6.66-6.61 (m, 2H), 6.39 (d, J = 8.5 Hz, 1H), 5.15-5.10 (m, 1H), 3.98-3.54 (m, 5H), 3.48-3.34 (m, 1H), 3.33-3.15 (m, 2H), 3.14-2.97 (m, 3H), 2.45 (s, 3H), 2.38 (s, 3H), 1.65 (d, J = 6.7 Hz, 3H). |
| 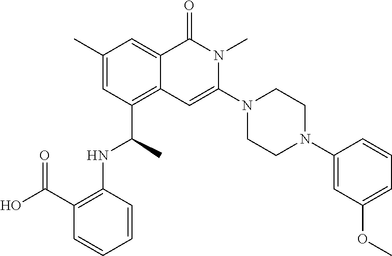 [SMILES: COc1cccc(N2CCN(c3cc4c([C@@H](C)Nc5ccccc5C(=O)O)cc(C)cc4c(=O)n3C)CC2)c1] C₃₁H₃₄N₄O₄ | 147 | | ES+ 527.1 [M + H] | ¹H NMR (400 MHz, Methanol-d₄) δ 8.08- 7.81 (m, 2H), 7.54 (s, 1H), 7.19-7.10 (m, 2H), 6.67-6.32 (m, 6H), 5.19-5.10 (m, 1H), 3.78 (s, 3H), 3.66 (s, 3H), 3.60 (s, 1H), 3.25 (s, 4H), 3.01 (s, 3H), 2.38 (s, 3H), 1.65- 1.58 (m, 3H). |
| 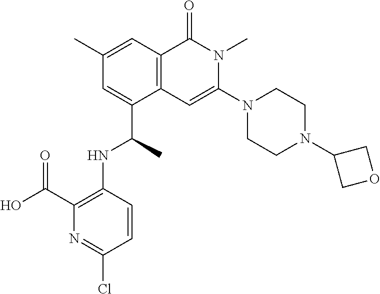 [SMILES: Cc1cc([C@@H](C)Nc2ccc(Cl)nc2C(=O)O)c2cc(N3CCN(C4COC4)CC3)n(C)c(=O)c2c1] C₂₆H₃₀ClN₅O₄ | 148 | | ES+ 512.0 [M + H] | ¹H NMR (400 MHz, DMSO-d₆) δ 8.35 (d, J = 6.2 Hz, 1H), 7.89 (s, 1H), 7.41 (d, J = 1.9 Hz, 1H), 7.36 (d, J = 9.0 Hz, 1H), 6.93 (d, J = 9.1 Hz, 1H), 6.36 (s, 1H), 5.28- 5.20 (m, 1H), 4.78- 4.52 (m, 4H), 3.50- 3.22 (m, 5H), 3.05- 2.85 (m, 7H), 2.33 (s, 3H), 1.56 (d, J = 6.5 Hz, 3H). |
| 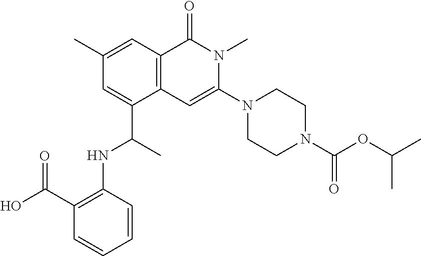 [SMILES: Cc1cc(C(C)Nc2ccccc2C(=O)O)c2cc(N3CCN(C(=O)OC(C)C)CC3)n(C)c(=O)c2c1] C₂₈H₃₄N₄O₅ | 149 | enantiomer 2 | ES− 505.2 [M − H] | ¹H NMR (300 MHz, DMSO-d₆) δ 8.53 (s, 1H), 7.88 (d, J = 1.6 Hz, 1H), 7.82-7.78 (m, 1H), 7.42 (d, J = 1.9 Hz, 1H), 7.20- 7.10 (m, 1H), 6.55- 6.44 (m, 1H), 6.41 (s, 1H), 6.35 (d, J = 8.5 Hz, 1H), 5.18- 5.14 (m, 1H), 4.85- 4.60 (m, 1H), 3.95 (s, 2H), 3.52 (s, 3H), 3.32-2.75 (m, 6H), 2.32 (s, 3H), 1.52 (d, J = 6.5 Hz, 3H), 1.22 (d, J = 6.2 Hz, 6H). |
| 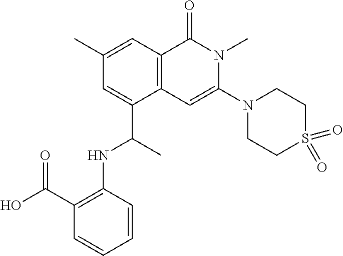 [SMILES: Cc1cc(C(C)Nc2ccccc2C(=O)O)c2cc(N3CCS(=O)(=O)CC3)n(C)c(=O)c2c1] C₂₄H₂₇N₃O₅S | 150 | enantiomer 1 | ES− 468.1 [M − H] | ¹H NMR (300 MHz, DMSO-d₆) δ 12.70 (s, 1H), 8.46 (s, 1H), 7.88 (d, J = 1.8 Hz, 1H), 7.85-7.75 (m, 1H), 7.41 (d, J = 1.9 Hz, 1H), 7.22-7.10 (m, 1H), 6.58 (s, 1H), 6.61-6.37 (m, 1H), 6.35 (d, J = 8.5 Hz, 1H), 5.23 (s, 1H), 3.55 (s, 3H), 3.54-3.50 (m, 4H), 3.46-3.43 (m, 4H), 2.32 (s, 3H), 1.51 (d, J = 6.5 Hz, 3H). |
| 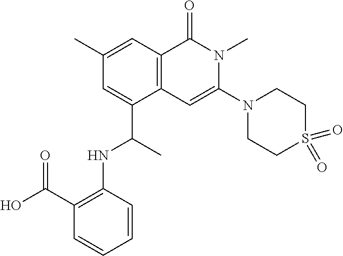 [SMILES: Cc1cc(C(C)Nc2ccccc2C(=O)O)c2cc(N3CCS(=O)(=O)CC3)n(C)c(=O)c2c1] C₂₄H₂₇N₃O₅S | 151 | enantiomer 2 | ES− 468.1 [M − H] | ¹H NMR (300 MHz, DMSO-d₆) δ 12.70 (s, 1H), 8.38 (s, 1H), 7.88 (d, J = 1.7 Hz, 1H), 7.85-7.75 (m, 1H), 7.41 (d, J = 1.9 Hz, 1H), 7.22-7.11 (m, 1H), 6.59 (s, 1H), 6.59 (m, 1H), 6.37 (d, J = 8.4 Hz, 1H), 5.54-4.84 (m, 1H), 3.55 (s, 3H), 3.53-3.49 (m, 4H), 3.47-3.42 (m, 4H), 2.32 (s, 3H), 1.52 (d, J = 6.5 Hz, 3H). |
| 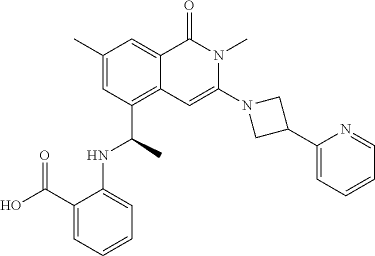 [SMILES: Cc1cc([C@@H](C)Nc2ccccc2C(=O)O)c2cc(N3CC(c4ccccn4)C3)n(C)c(=O)c2c1] C₂₈H₂₈N₄O₃ | 152 | | ES+ 469.3 [M + H] | ¹H NMR (DMSO-d₆, 300 MHz, ppm): δ 8.60-8.57 (m, 1H), 8.42 (s, 1H), 7.85- 7.73 (m, 3H), 7.54- 7.15 (m, 4H), 6.52- 6.48 (m, 1H), 6.36 (d, J = 8.5 Hz, 1H), 5.92 (s, 1H), 5.10 (d, J = 6.8 Hz, 1H), 4.45- 4.25 (m, 2H), 4.21- 4.04 (m, 3H), 3.45 (s, 3H), 2.30 (s, 3H), 1.53 (d, J = 6.5 Hz, 3H). |
| 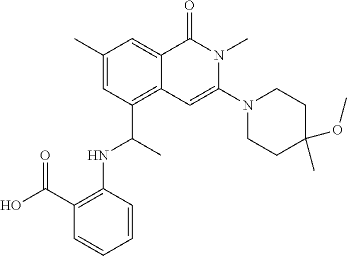 [SMILES: COC1(C)CCN(c2cc3c(C(C)Nc4ccccc4C(=O)O)cc(C)cc3c(=O)n2C)CC1] C₂₇H₃₃N₃O₄ | 153 | enantiomer 1 | ES+ 464.1 [M + H] | ¹H NMR (300 MHz, DMSO-d₆) δ 12.50 (s, 1H), 8.38 (s, 1H), 7.87-7.79 (m, 2H), 7.42-7.39 (d, J = 2.0 Hz, 1H), 7.22- 7.17 (m, 1H), 6.54- 6.49 (m, 1H), 6.40- 6.33 (m, 2H), 5.16- 5.13 (m, 1H), 3.48 (s, 3H), 3.14 (s, 3H), 2.89-2.73 (m, 4H), 2.32 (s, 3H), 1.82- 1.80 (m, 2H), 1.69- 1.59 (m, 2H), 1.52 (d, J = 6.4 Hz, 3H), 1.16 (s, 3H). |
| 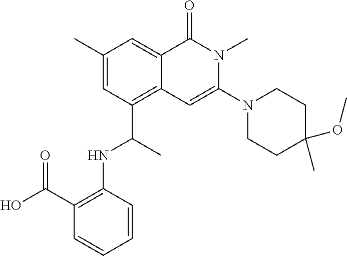 [SMILES: COC1(C)CCN(c2cc3c(C(C)Nc4ccccc4C(=O)O)cc(C)cc3c(=O)n2C)CC1] C₂₇H₃₃N₃O₄ | 154 | enantiomer 2 | ES+ 464.1 [M + H] | ¹H NMR (300 MHz, DMSO-d₆) δ 12.30 (s, 1H), 8.39 (s, 1H), 7.86-7.79 (m, 2H), 7.42-7.38 (d, J = 1.9 Hz, 1H), 7.21- 7.17 (m, 1H), 6.54- 6.49 (m, 1H), 6.39- 6.30 (m, 2H), 5.15- 5.12 (m, 1H), 3.52 (s, 3H), 3.14 (s, 3H), 2.89-2.87 (m, 4H), 2.31 (s, 3H), 1.81- 1.79 (m, 2H), 1.70- 1.64 (m, 2H), 1.52 (d, J = 6.4 Hz, 3H), 1.17 (s, 3H). |
| 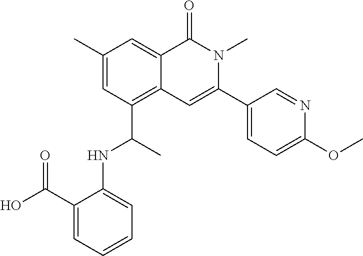 [SMILES: COc1ccc(-c2cc3c(C(C)Nc4ccccc4C(=O)O)cc(C)cc3c(=O)n2C)cn1] C₂₆H₂₅N₃O₄ | 155 | enantiomer 1 | ES− 442.1 [M − H] | ¹H NMR (400 MHz, DMSO-d₆, ppm) 8 12.79 (s, 1H), 8.44- 8.39 (m, 2H), 8.02- 7.95 (m, 2H), 7.80- 7.78 (m, 1H), 7.48 (d, J = 1.9 Hz, 1H), 7.16 (m, 1H), 6.97 (d, J = 8.6 Hz, 1H), 6.90 (s, 1H), 6.56- 6.47 (m, 1H), 6.34 (d, J = 8.5 Hz, 1H), 5.27-5.19 (m, 1H), 3.93 (s, 3H), 3.36 (s, 3H), 2.37 (s, 3H), 1.51 (d, J = 6.5 Hz, 3H). |
| 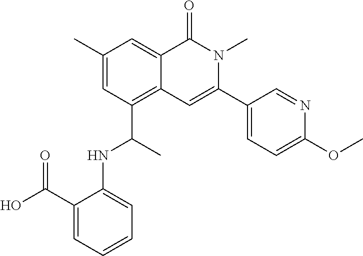 [SMILES: COc1ccc(-c2cc3c(C(C)Nc4ccccc4C(=O)O)cc(C)cc3c(=O)n2C)cn1] C₂₆H₂₅N₃O₄ | 156 | enantiomer 2 | ES− 442.1 [M − H] | ¹H NMR (400 MHz, DMSO-d₆, ppm) 8 8.47-8.41 (m, 2H), 8.02-7.95 (m, 2H), 7.80-7.78 (m, 1H), 7.49 (d, J = 1.9 Hz, 1H), 7.16 (m, 1H), 6.97 (d, J = 8.6 Hz, 1H), 6.90 (s, 1H), 6.55-6.47 (m, 1H), 6.33 (d, J = 8.5 Hz, 1H), 5.27-5.19 (m, 1H), 3.93 (s, 3H), 3.36 (s, 3H), 2.37 (s, 3H), 1.51 (d, J = 6.5 Hz, 3H). |
| 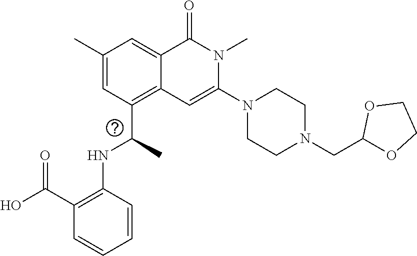 [SMILES: Cc1cc([C@@H](C)Nc2ccccc2C(=O)O)c2cc(N3CCN(CC4OCCO4)CC3)n(C)c(=O)c2c1] C₂₈H₃₄N₄O₅ | 157 | | ES+ 507.6 [M + H] | ¹H NMR (300 MHz, DMSO-d₆) δ 8.36 (d, J = 5.9 Hz, 1H), 7.87 (d, J = 1.7 Hz, 1H), 7.81-7.79 (m, 1H), 7.42 (d, J = 1.9 Hz, 1H), 7.21-7.19 (m, 1H), 6.55-6.50 (m, 1H), 6.44-6.33 (m, 2H), 5.15-5.13 (m, 1H), 4.95-4.93 (m, 1H), 3.98-3.70 (m, 4H), 3.48 (s, 3H), 2.94 (s, 6H), 2.58 (d, J = 4.4 Hz, 2H), 2.32 (s, 3H), 2.07 (s, 2H), 1.54 (d, J = 6.5 Hz, 3H). |
| 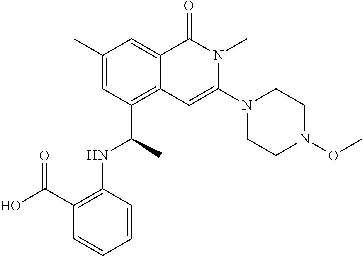 [SMILES: CON1CCN(c2cc3c([C@@H](C)Nc4ccccc4C(=O)O)cc(C)cc3c(=O)n2C)CC1] C₂₅H₃₀N₄O₄ | 158 | | ES+ 451.5 [M + H] | ¹H NMR (300 MHz, DMSO-d₆) δ 12.73 (s, 1H), 8.37 (d, J = 5.9 Hz, 1H), 7.88- 7.86 (m, 1H), 7.81- 7.79 (m, 1H), 7.41 (d, J = 1.9 Hz, 1H), 7.20-7.18 (m, 1H), 6.50-6.53 (m, 1H), 6.38 (d, J = 9.1 Hz, 2H), 5.21-5.11 (m, 1H), 3.48-3.44 (m, 6H), 3.19 (s, 3H), 2.89-2.71 (m, 4H), 2.69 (s, 1H), 2.32 (s, 3H), 1.53 (d, J = 6.5 Hz, 3H). |
| 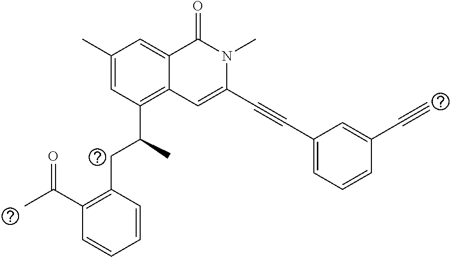 [SMILES: C#Cc1cccc(C#Cc2cc3c([C@@H](C)Cc4ccccc4C(C)=O)cc(C)cc3c(=O)n2C)c1] C₂₉H₂₃N₃O₃ | 159 | | ES− 459.9 [M − H] | ¹H NMR (400 MHz, Chloroform-d) 8 8.25-8.11 (m, 2H), 8.05-7.92 (m, 1H), 7.88-7.85 (m, 1H), 7.81-7.75 (m, 1H), 7.69 (m, 1H), 7.58- 7.49 (m, 2H), 7.24 (s, 1H), 7.19-7.15 (m, 1H), 6.61-6.52 (m, 1H), 6.26-6.19 (m, 1H), 5.07-5.02 (m, 1H), 3.84 (s, 3H), 2.43 (s, 3H), 1.68 (d, J = 6.8 Hz, 3H). |
| 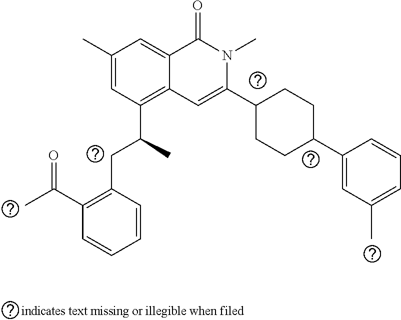 [SMILES: CC(=O)c1ccccc1C[C@H](C)c1cc(C)cc2c(=O)n(C)c(C3CCC(c4cccc(C)c4)CC3)cc12] C₃₀H₃₁ClN₄O₃ | 160 | | ES− 529.1 [M − H] | ¹H NMR (400 MHz, Methanol-d₄) δ 8.01- 7.98 (m, 1H), 7.89- 7.85 (m, 1H), 7.53 (s, 1H), 7.20-7.16 (m, 1H), 7.14-7.10 (m, 1H), 6.97-6.92 (m, 1H), 6.90-6.85 (m, 1H), 6.82-6.79 (m, 1H), 6.56-6.46 (m, 2H), 6.39-6.32 (m, 1H), 5.15-5.08 (m, 1H), 3.83-3.51 (s, 5H), 3.25-2.85 (m, 6H), 2.36 (s, 3H), 1.74-1.56 (m, 3H). |
| 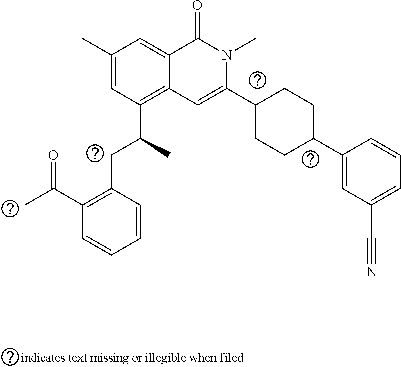 [SMILES: CC(=O)c1ccccc1C[C@H](C)c1cc(C)cc2c(=O)n(C)c(C3CCC(c4cccc(C#N)c4)CC3)cc12] C₃₁H₃₁N₅O₃ | 161 | | ES− 520.1 [M − H] | ¹H NMR (400 MHz, Methanol-d₄) δ 8.02- 7.98 (m, 1H), 7.95- 7.89 (m, 1H), 7.54- 7.49 (m, 1H), 7.45- 7.37 (m, 1H), 7.33- 7.30 (m, 2H), 7.2- 7.08 (m, 2H), 6.57 (s, 1H), 6.53-6.49 (m, 1H), 6.42-6.35 (m, 1H), 5.12-5.09 (m, 1H), 3.86-3.70 (m, 2H), 3.67 (s, 3H), 3.06 (s, 6H), 2.39 (s, 3H), 1.65- 1.55 (m, 3H). |
| 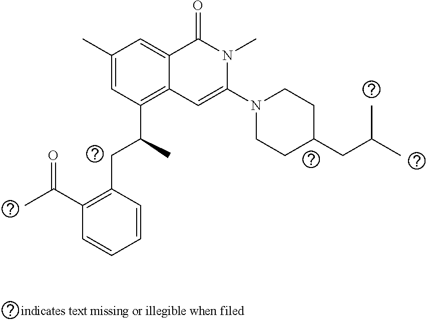 C₂₆H₃₀F₂N₄O₃ | 162 | | ES+ 485.3 [M + H] | ¹H NMR (400 MHz, Methanol-d₄) δ 8.02- 7.91 (m, 1H), 7.89- 7.85 (m, 1H), 7.53- 7.50 (m, 1H), 7.19- 7.09 (m, 1H), 6.54- 6.49 (m, 2H), 6.38- 6.35 (m, 1H), 6.25- 5.91 (m, 1H), 5.15- 5.09 (m, 1H), 3.62 (s, 3H), 3.24-2.64 (m, 10H), 2.37 (s, 3H), 1.65-1.55 (m, 3H). |
| 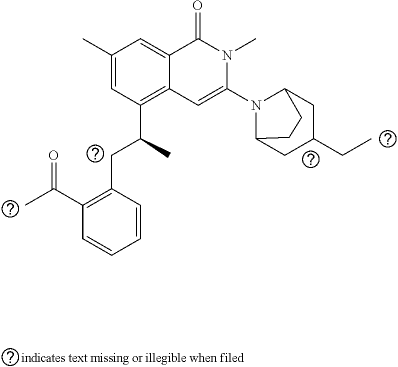 C₂₈H₃₁F₃N₄O₃ | 163 | | ES+ 529.1 [M + H] | ¹H NMR (400 MHz, DMSO-d₆) δ 8.40 (s, 1H), 7.84-7.78 (m, 2H), 7.42 (d, J = 1.9 Hz, 1H), 7.21-7.17 (m, 1H), 6.54-6.50 (m, 1H), 6.42 (d, J = 8.5 Hz, 1H), 6.13 (s, 1H), 5.03-5.01 (m, 1H), 3.84-3.82 (m, 2H), 3.56 (s, 3H), 3.39-3.22 (m, 2H), 2.86-2.76 (m, 4H), 2.32 (s, 3H), 1.94- 1.78 (m, 4H), 1.54 (d, J = 6.5 Hz, 3H). |
| 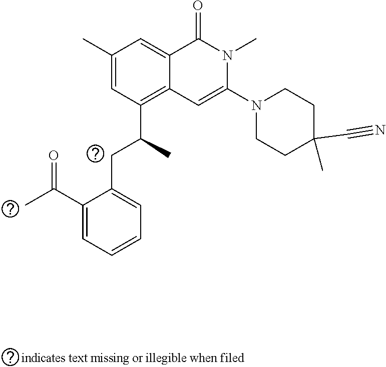 C₂₇H₃₀N₄O₃ | 164 | | ES+ 459.1 [M + H] | ¹H NMR (400 MHz, DMSO-d₆) δ 8.86 (s, 1H), 7.86-7.78 (m, 2H), 7.42 (d, J = 1.9 Hz, 1H), 7.11-7.07 (m, 1H), 6.48-6.29 (m, 3H), 5.17-5.15 (m, 1H), 3.65 (s, 3H), 3.33-2.81 (m, 4H), 2.31 (s, 3H), 2.04-1.99 (m, 2H), 1.82-1.76 (m, 2H), 1.52 (d, J = 6.6 Hz, 3H), 1.44 (s, 3H). |
| 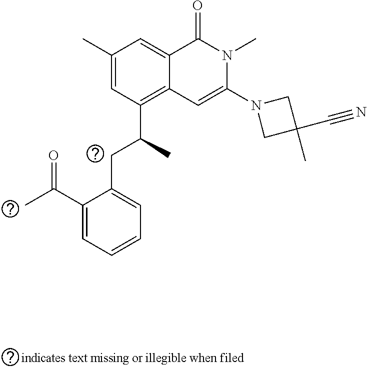 C₂₅H₂₆N₄O₃ | 165 | | ES+ 431.2 [M + H] | ¹H NMR (400 MHz, DMSO-d₆) δ 8.45 (s, 1H), 7.84-7.77 (m, 2H), 7.37 (d, J = 1.9 Hz, 1H), 7.21-7.15 (m, 1H), 6.55-6.47 (m, 1H), 6.34 (d, J = 8.4 Hz, 1H), 5.95 (s, 1H), 5.20-5.12 (m, 1H), 4.33-4.27 (m, 2H), 3.97-3.92 (m, 2H), 3.41 (s, 3H), 2.29 (s, 3H), 1.69 (s, 3H), 1.52 (d, J = 6.6 Hz, 3H). |
| 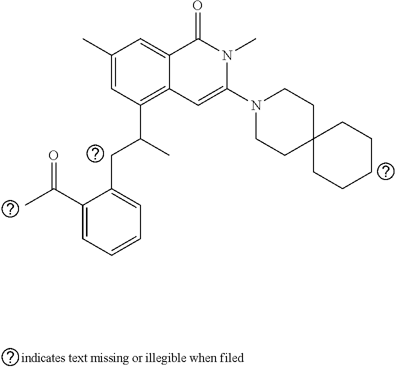 C₂₉H₃₅N₃O₄ | 166 | enantiomer 1 | ES+ 490.1 [M + H] | ¹H NMR (300 MHz, DMSO-d₆) δ 8.40 (s, 1H), 7.86 (d, J = 1.9 Hz, 1H), 7.82-7.76 (m, 1H), 7.40 (d, J = 1.9 Hz, 1H), 7.23- 7.18 (m, 1H), 6.55- 6.51 (m, 1H), 6.38 (d, J = 8.0 Hz, 2H), 5.18-5.12 (m, 1H), 3.61-3.58 (m, 4H), 3.55 (s, 3H), 3.05- 2.89 (m, 4H), 2.32 (s, 3H), 1.72-1.54 (m, 11H). |
| 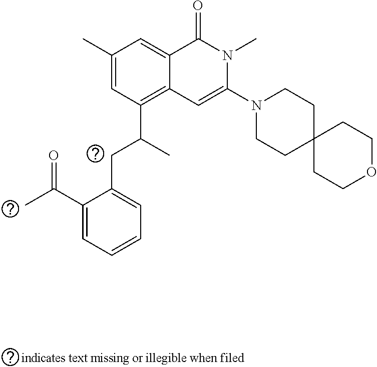 C₂₉H₃₅N₃O₄ | 167 | enantiomer 2 | ES+ 490.1 [M + H] | ¹H NMR (300 MHz, DMSO-d₆) δ 8.44 (s, 1H), 7.86 (d, J = 1.8 Hz, 1H), 7.82-7.76 (m, 1H), 7.40 (d, J = 1.9 Hz, 1H), 7.24- 7.12 (m, 1H), 6.55- 6.50 (m, 1H), 6.42- 6.33 (m, 2H), 5.18- 5.12 (m, 1H), 3.61- 3.58 (m, 4H), 3.48 (s, 3H), 3.05-2.89 (m, 4H), 2.32 (s, 3H), 1.72-1.54 (m, 11H). |
| 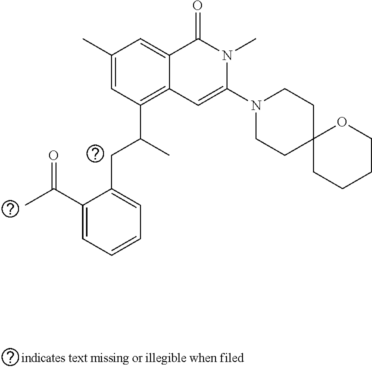 C₂₉H₃₅N₃O₄ | 168 | enantiomer 1 | ES+ 490.3 [M + H] | ¹H NMR (300 MHz, Methanol-d₄) δ 7.9 (s, 1H), 7.94-7.85 (m, 1H), 7.52 (d, J = 1.9 Hz, 1H), 7.19- 7.08 (m, 1H), 6.58- 6.48 (m, 1H), 6.47 (s, 1H), 6.36 (d, J = 8.5 Hz, 1H), 5.15- 5.02 (m, 1H), 3.75- 3.66 (m, 2H), 3.62 (s, 3H), 2.95 (s, 4H), 2.37 (s, 3H), 2.09 (s, 2H), 1.74-1.67 (m, 3H), 1.63 (d, J = 6.7 Hz, 3H), 1.57-1.45 (m, 5H). |
| 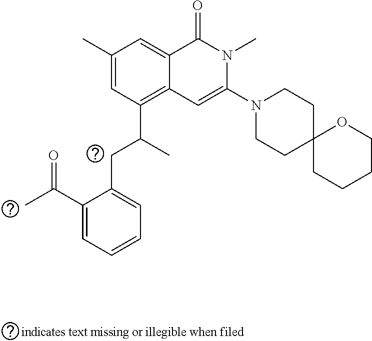 C₂₉H₃₅N₃O₄ | 169 | enantiomer 2 | ES+ 490.2 [M + H] | ¹H NMR (300 MHz, Methanol-d₄) δ 7.96 (s, 1H), 7.93-7.85 (m, 1H), 7.52 (d, J = 1.9 Hz, 1H), 7.20- 7.08 (m, 1H), 6.58- 6.48 (m, 1H), 6.47 (s, 1H), 6.36 (d, J = 8.5 Hz, 1H), 5.15- 5.03 (m, 1H), 3.75- 3.66 (m, 2H), 3.62 (s, 3H), 2.95 (s, 4H), 2.37 (s, 3H), 2.09 (s, 2H), 1.74-1.67 (m, 3H), 1.63 (d, J = 6.7 Hz, 3H), 1.57-1.45 (m, 5H). |
| 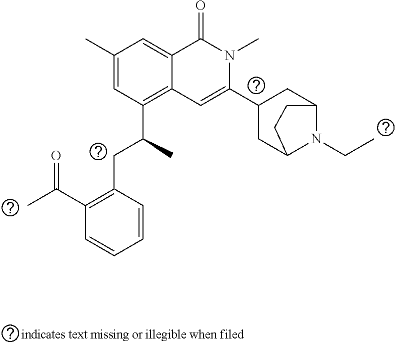 C₂₈H₃₁F₃N₄O₃ | 170 | | ES+ 529.1 [M + H] | ¹H NMR (300 MHz, DMSO-d₆) δ 12.71 (s, 1H), 8.36 (d, J = 5.8 Hz, 1H), 7.87 (d, J = 1.7 Hz, 1H), 7.85- 7.50 (m, 1H), 7.39 (d, J = 1.9 Hz, 1H), 7.37-6.64 (m, 1H), 6.57-6.44 (m, 2H), 6.36 (d, J = 8.4 Hz, 1H), 5.68-4.92 (m, 1H), 3.56 (s, 3H), 3.54-3.20 (m, 2H), 3.17-3.10 (m, 2H), 3.06-3.02 (m, 1H), 2.98-2.90 (m, 3H), 2.31 (s, 3H), 1.94- 1.85 (m, 4H), 1.51 (d, J = 6.5 Hz, 3H). |
| 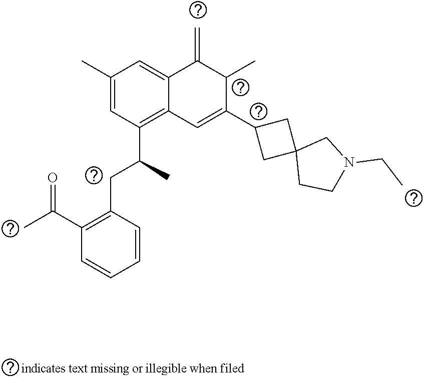 C₂₈H₃₁F₃N₄O₃ | 171 | | ES+ 529.1 [M + H] | ¹H NMR (400 MHz, DMSO-d₆) δ 8.58 (s, 1H), 7.83-7.76 (m, 2H), 7.36 (d, J = 1.9 Hz, 1H), 7.18-7.13 (m, 1H), 6.52-6.47 (m, 1H), 6.32 (d, J = 8.5 Hz, 1H), 5.84 (s, 1H), 5.12-5.02 (m, 1H), 3.91-3.84 (m, 4H), 3.35-3.25 (m, 5H), 2.96 (s, 2H), 2.79-2.71 (m, 2H), 2.28 (s, 3H), 2.13- 2.03 (m, 2H), 1.52 (d, J = 6.5 Hz, 3H). |
| 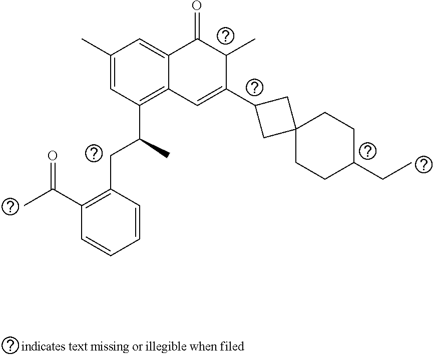 C₂₉H₃₃F₃N₄O₃ | 172 | | ES+ 543.2 [M + H] | ¹H NMR (DMSO-d₆, 300 MHz, ppm): δ 8.76 (s, 1H), 7.85- 7.75 (m, 2H), 7.36 (d, J = 1.9 Hz, 1H), 7.16-7.04 (m, 1H), 6.50-6.45 (m, 1H), 6.31 (d, J = 8.4 Hz, 1H), 5.82 (s, 1H), 5.05-5.00 (m, 1H), 3.72-3.61 (m, 4H), 3.39 (s, 3H), 3.22- 3.05 (m, 2H), 2.62- 2.56 (m, 4H), 2.28 (s, 3H), 1.79-1.74 (m, 4H), 1.52 (d, J = 6.4 Hz, 3H). |
| 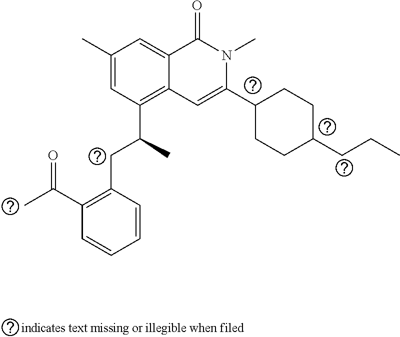 [SMILES: CCCC1CCC(c2cc3c([C@@H](C)Cc4ccccc4C(C)=O)cc(C)cc3c(=O)n2C)CC1] C₂₆H₃₂N₄O₄ | 173 | | ES− 463.0 [M − H] | ¹H NMR (DMSO-d₆, 400 MHz, ppm): δ 8.40 (s, 1H), 7.87 (d, J = 1.7 Hz, 2H), 7.41 (d, J = 1.9 Hz, 1H), 7.25-7.15 (m, 1H), 6.57-6.46 (m, 1H), 6.43-6.33 (m, 2H), 5.20-5.13 (m, 1H), 3.80-3.65 (m, 2H), 3.49 (s, 3H), 3.18 (s, 4H), 2.89-2.60 (m, 4H), 2.32 (s, 3H), 1.53 (d, J = 6.4 Hz, 3H), 1.15 (m, 3H). |
| 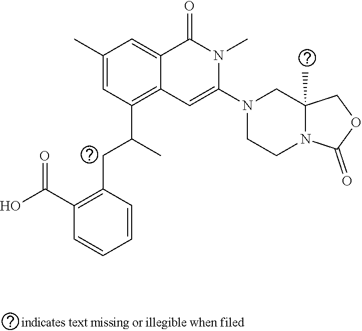 [SMILES: Cc1cc(C(C)Cc2ccccc2C(=O)O)c2cc(N3CCN4C(=O)OC[C@]4(C)C3)n(C)c(=O)c2c1] C₂₆H₂₈N₄O₅ | 174 | diastereomer 3 | ES− 475.2 [M − H] | ¹H NMR (400 MHz, Methanol-d₄) δ 8.01- 7.96 (m, 1H), 7.89- 7.85 (m, 1H), 7.55 (s, 1H), 7.15-7.10 (m, 1H), 6.57 (s, 1H), 6.53-6.49 (m, 1H), 6.45-6.38 (m, 1H), 5.15-5.08 (m, 1H), 4.62-4.49 (m, 1H), 4.22-4.04 (m, 2H), 3.84-3.80 (m, 1H), 3.65 (s, 3H), 3.31-3.09 (m, 2H), 3.21-3.16 (m, 1H), 2.78-2.63 (m, 2H), 2.39 (s, 3H), 1.64 (d, J = 6.7 Hz, 3H). |
| 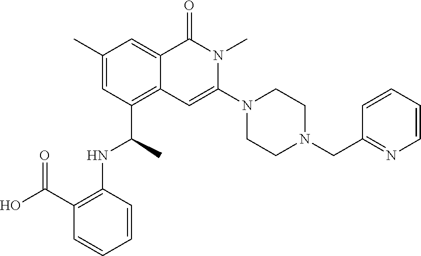 [SMILES: Cc1cc([C@@H](C)Nc2ccccc2C(=O)O)c2cc(N3CCN(Cc4ccccn4)CC3)n(C)c(=O)c2c1] C₃₀H₃₃N₅O₃ | 175 | | ES+ 512.1 [M + H] | ¹H NMR (400 MHz, Methanol-d₄) δ 8.56- 8.50 (m, 1H), 7.97 (s, 1H), 7.92-7.81 (m, 2H), 7.62-7.56 (m, 1H), 7.55-7.49 (m, 1H), 7.40-7.35 (m, 1H), 7.16-7.09 (m, 1H), 6.56-6.47 (m, 2H), 6.40-6.35 (m, 1H), 5.15-5.08 (m, 1H), 3.84 (s, 2H), 3.62 (s, 3H), 3.19-2.85 (m, 6H), 2.80-2.55 (m, 2H), 2.37 (s, 3H), 1.70- 1.60 (m, 3H). |
| 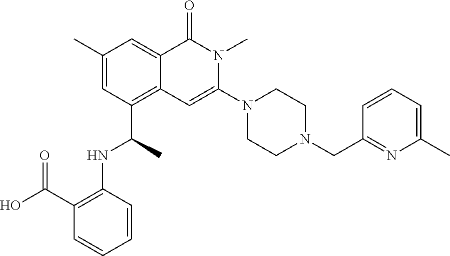 [SMILES: Cc1cc([C@@H](C)Nc2ccccc2C(=O)O)c2cc(N3CCN(Cc4cccc(C)n4)CC3)n(C)c(=O)c2c1] C₃₁H₃₅N₅O₃ | 176 | | ES+ 526.1 [M + H] | ¹H NMR (400 MHz, Methanol-d₄) δ 7.97 (s, 1H), 7.88-7.86 (m, 1H), 7.75-7.70 (m, 1H), 7.60-7.52 (m, 1H), 7.42-7.35 (m, 1H), 7.25-7.20 (m, 1H), 7.15-7.10 (m, 1H), 6.58-6.44 (m, 2H), 6.39-6.35 (m, 1H), 5.15-5.08 (m, 1H), 3.78 (s, 2H), 3.62 (s, 3H), 3.05-3.00 (m, 6H), 2.54 (s, 5H), 2.37 (s, 3H), 1.65-1.59 (m, 3H). |
| 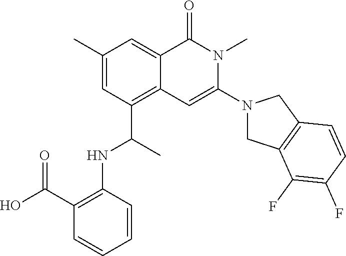 [SMILES: Cc1cc(C(C)Nc2ccccc2C(=O)O)c2cc(N3Cc4ccc(F)c(F)c4C3)n(C)c(=O)c2c1] C₂₈H₂₅F₂N₃O₃ | 177 | enantiomer 1 | ES+ 490.2 [M + H] | ¹H NMR (400 MHz, Methanol-d₄) δ 8.01- 7.96 (m, 1H), 7.88- 7.87 (m, 1H), 7.54 (d, J = 1.9 Hz, 1H), 7.26-7.17 (m, 1H), 7.17-7.07 (m, 2H), 6.78 (s, 1H), 6.51- 6.49 (m, 1H), 6.40 (d, J = 8.7 Hz, 1H), 5.13-5.11 (m, 1H), 4.65 (s, 2H), 4.55 (s, 2H), 3.69 (s, 3H), 2.38 (s, 3H), 1.63 (d, J = 6.7 Hz, 3H). |
| 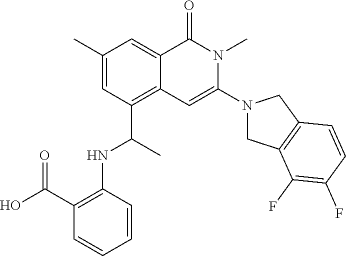 [SMILES: Cc1cc(C(C)Nc2ccccc2C(=O)O)c2cc(N3Cc4ccc(F)c(F)c4C3)n(C)c(=O)c2c1] C₂₈H₂₅F₂N₃O₃ | 178 | enantiomer 2 | ES+ 490.2 [M + H] | ¹H NMR (400 MHz, Methanol-d₄) δ 8.00- 7.95 (m, 1H), 7.88- 7.87 (m, 1H), 7.55 (d, J = 1.9 Hz, 1H), 7.23-7.17 (m, 1H), 7.15-7.05 (m, 2H), 6.78 (s, 1H), 6.51- 6.49 (m, 1H), 6.36 (d, J = 8.4 Hz, 1H), 5.13-5.11 (m, 1H), 4.65 (s, 2H), 4.55 (s, 2H), 3.69 (s, 3H), 2.37 (s, 3H), 1.62 (d, J = 6.7 Hz, 3H). |
| 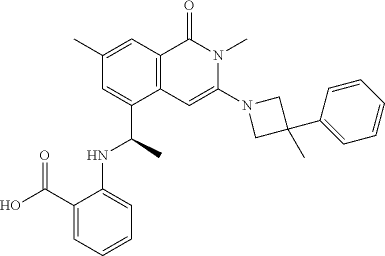 [SMILES: Cc1cc([C@@H](C)Nc2ccccc2C(=O)O)c2cc(N3CC(C)(c4ccccc4)C3)n(C)c(=O)c2c1] C₃₀H₃₁N₃O₃ | 179 | | ES+ 482.3 [M + H] | ¹H NMR (DMSO-d₆, 400 MHz, ppm): δ 8.80 (s, 1H), 7.82- 7.77 (m, 2H), 7.42- 7.34 (m, 3H), 7.34- 7.28 (m, 2H), 7.28- 7.20 (m, 1H), 7.09 (s, 1H), 6.50-6.41 (m, 1H), 6.30 (d, J = 8.4 Hz, 1H), 5.91 (s, 1H), 5.09-5.05 (m, 1H), 4.19-4.05 (m, 2H), 4.01-3.90 (m, 2H), 3.45 (s, 3H), 2.28 (s, 3H), 1.69 (s, 3H), 1.50 (d, J = 6.6 Hz, 3H). |
| 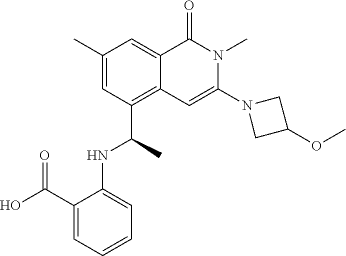 [SMILES: COC1CN(c2cc3c([C@@H](C)Nc4ccccc4C(=O)O)cc(C)cc3c(=O)n2C)C1] C₂₄H₂₇N₃O₄ | 180 | | ES+ 422.2 [M + H] | ¹H NMR (400 MHz, DMSO-d₆) δ 9.73 (s, 1H), 7.78 (d, J = 9.3 Hz, 2H), 7.39 (d, J = 1.9 Hz, 1H), 6.95- 6.86 (m, 1H), 6.39- 6.31 (m, 1H), 6.27 (s, 1H), 6.12 (d, J = 8.2 Hz, 1H), 5.91 (s, 1H), 4.97-4.96 (m, 1H), 4.30-4.22 (m, 1H), 4.20-4.11 (m, 2H), 3.79-3.75 (m, 2H), 3.40 (s, 3H), 3.26 (s, 3H), 2.27 (s, 3H), 1.46 (d, J = 6.6 Hz, 3H). |
| 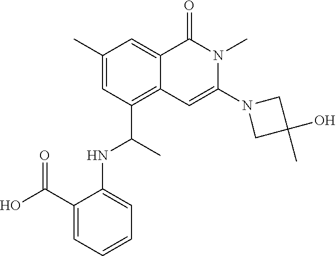 [SMILES: Cc1cc(C(C)Nc2ccccc2C(=O)O)c2cc(N3CC(C)(O)C3)n(C)c(=O)c2c1] C₂₄H₂₇N₃O₄ | 181 | enantiomer 1 | ES+ 422.1 [M + H] | ¹H NMR (300 MHz, Chloroform-d) 8 8.16 (s, 1H), 8.07 (s, 1H), 8.00 (d, J = 7.9 Hz, 1H), 7.45 (s, 1H), 7.25-7.21 (m, 1H), 6.62-6.58 (m, 1H), 6.37 (d, J = 8.5 Hz, 1H), 5.91 (s, 1H), 5.05-4.95 (m, 1H), 3.85-3.77 (m, 4H), 3.54 (s, 3H), 2.38 (s, 3H), 1.65 (d, J = 5.4 Hz, 3H), 1.25-1.10 (m, 4H). |
| 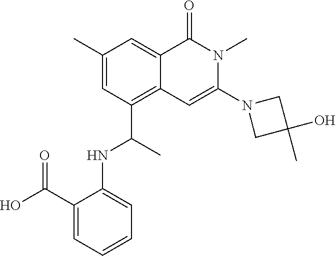 [SMILES: Cc1cc(C(C)Nc2ccccc2C(=O)O)c2cc(N3CC(C)(O)C3)n(C)c(=O)c2c1] C₂₄H₂₇N₃O₄ | 182 | enantiomer 2 | ES+ 422.2 [M + H] | ¹H NMR (300 MHz, Chloroform-d) 8 8.17 (s, 1H), 8.07 (s, 1H), 8.00 (d, J = 8.1, Hz, 1H), 7.45 (s, 1H) 7.26-7.21 (m, 1H), 6.62-6.58 (m, 1H), 6.37 (d, J = 8.6 Hz, 1H), 5.91 (s, 1H), 5.05-4.95 (m, 1H), 3.85-3.77 (m, 4H), 3.54 (s, 3H), 2.38 (s, 3H), 1.65 (d, J = 5.4 Hz, 3H), 1.26-1.11 (m, 4H), |
| 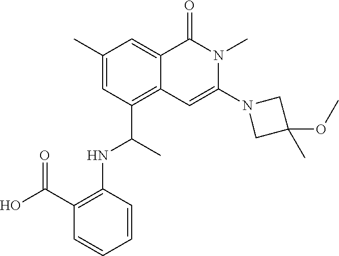 [SMILES: COC1(C)CN(c2cc3c(C(C)Nc4ccccc4C(=O)O)cc(C)cc3c(=O)n2C)C1] C₂₅H₂₉N₃O₄ | 183 | enantiomer 1 | ES+ 436.2 [M + H] | ¹H NMR (300 MHz, DMSO-d₆) δ 8.67 (s, 1H), 7.80-7.78 (m, 2H), 7.37 (d, J = 1.9 Hz, 1H), 7.15-7.12 (m, 1H), 6.53-6.42 (m, 1H), 6.33 (d, J = 8.3 Hz, 1H), 5.89 (s, 1H), 5.12-5.05 (m, 1H), 3.87-3.85 (m, 2H), 3.78-3.45 (m, 2H), 3.45 (s, 3H), 3.20 (s, 3H), 2.29 (s, 3H), 1.52 (d, J = 7.0 Hz, 6H). |
| 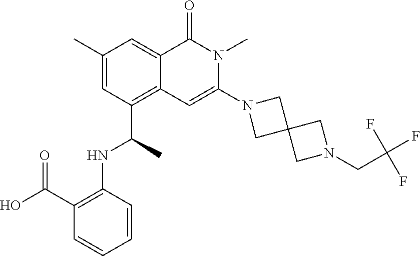 [SMILES: Cc1cc([C@@H](C)Nc2ccccc2C(=O)O)c2cc(N3CC4(CN(CC(F)(F)F)C4)C3)n(C)c(=O)c2c1] C₂₇H₂₉F₃N₄O₃ | 184 | | ES+ 515.2 [M + H] | ¹H NMR (DMSO-d₆, 400 MHz, ppm): δ 8.80 (s, 1H), 7.78 (s, 2H), 7.37 (d, J = 1.8 Hz, 1H), 7.06 (s, 1H), 6.45 (d, J = 7.7 Hz, 1H), 6.25 (d, J = 8.4 Hz, 1H), 5.84 (s, 1H), 5.01-4.99 (m, 1H), 4.09-3.99 (m, 4H), 3.58-3.48 (m, 4H), 3.39 (s, 3H), 3.21-3.17 (m, 2H), 2.27 (s, 3H), 1.51 (d, J = 6.5 Hz, 3H). |
| 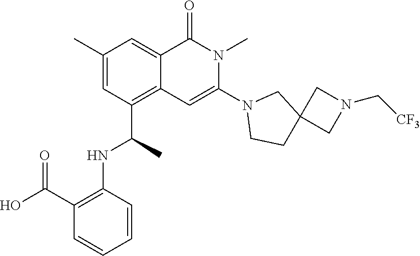 [SMILES: Cc1cc([C@@H](C)Nc2ccccc2C(=O)O)c2cc(N3CCC4(CN(CC(F)(F)F)C4)C3)n(C)c(=O)c2c1] C₂₈H₃₁F₃N₄O₃ | 185 | | ES+ 529.2 [M + H] | ¹H NMR (400 MHz, DMSO-d₆) δ 8.49 (s, 1H), 7.86-7.77 (m, 2H), 7.39 (d, J = 1.9 Hz, 1H), 7.17-7.10 (m, 1H), 6.51-6.45 (m, 1H), 6.36 (d, J = 8.5 Hz, 1H), 6.24 (s, 1H), 5.10-5.00 (m, 1H), 3.42-3.20 (m, 8H), 3.19-3.12 (m, 5H), 2.30 (s, 3H), 2.10 (d, J = 6.8 Hz, 2H), 1.53 (d, J = 6.5 Hz, 3H). |
| 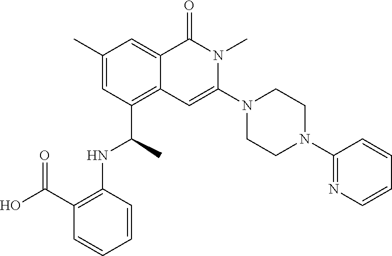 [SMILES: Cc1cc([C@@H](C)Nc2ccccc2C(=O)O)c2cc(N3CCN(c4ccccn4)CC3)n(C)c(=O)c2c1] C₂₉H₃₁N₅O₃ | 186 | | ES+ 498.3 [M + H] | ¹H NMR (400 MHz, Methanol-d₄) δ 8.21- 8.03 (m, 1H), 8.01- 7.95 (m, 1H), 7.95- 7.83 (m, 1H), 7.74- 7.66 (m, 1H), 7.66- 7.61 (m, 1H), 7.19- 7.03 (m, 1H), 6.97- 6.85 (m, 1H), 6.82- 6.64 (m, 1H), 6.54 (s, 1H), 6.55-6.42 (m, 1H), 6.41-6.33 (m, 1H), 5.21-5.04 (m, 1H), 4.24 (s, 2H), 3.68 (s, 3H), 3.30-2.69 (m, 6H), 2.37 (s, 3H), 1.63 (d, J = 6.7 Hz, 3H). |
| 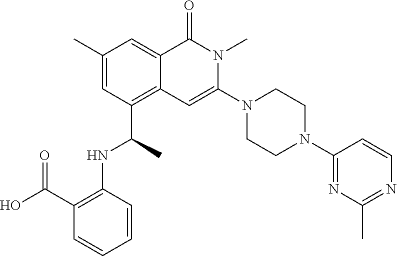 [SMILES: Cc1cc([C@@H](C)Nc2ccccc2C(=O)O)c2cc(N3CCN(c4ccnc(C)n4)CC3)n(C)c(=O)c2c1] C₂₉H₃₂N₆O₃ | 187 | | ES+ 513.1 [M + H] | ¹H NMR (400 MHz, Methanol-d₄) 8 8.12-8.01 (m, 1H), 7.99 (s, 1H), 7.89- 7.85 (m, 1H), 7.56- 7.52 (m, 1H), 7.15- 7.10 (m, 1H), 6.77- 6.72 (m, 1H), 6.58- 6.45 (m, 2H), 6.40- 6.35 (m, 1H), 5.15- 5.09 (m, 1H), 4.85- 4.52 (m, 2H), 3.70 (s, 3H), 3.31-3.29 (m, 4H), 3.08-2.75 (m, 2H), 2.48 (s, 3H), 2.38 (s, 3H), 1.63 (d, J = 6.4 Hz, 3H). |
| 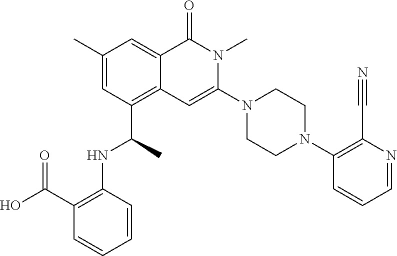 [SMILES: Cc1cc([C@@H](C)Nc2ccccc2C(=O)O)c2cc(N3CCN(c4cccnc4C#N)CC3)n(C)c(=O)c2c1] C₃₀H₃₀N₆O₃ | 188 | | ES− 521.2 [M − H] | ¹H NMR (400 MHz, Methanol-d₄) δ 8.32- 8.26 (m, 1H), 8.02- 7.96 (m, 1H), 7.92- 7.86 (m, 1H), 7.76- 7.70 (m, 1H), 7.62- 7.58 (m, 1H), 7.56- 7.53 (m, 1H), 7.20- 7.15 (m, 1H), 6.59 (s, 1H), 6.55-6.49 (m, 1H), 6.45-6.38 (m, 1H), 5.20-5.14 (m, 1H), 3.80-3.60 (m, 5H), 3.30-3.00 (m, 6H), 2.39 (s, 3H), 1.70-1.64 (m, 3H) |
| 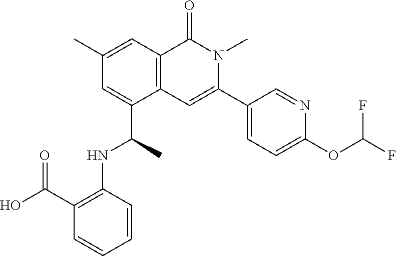 [SMILES: Cc1cc([C@@H](C)Nc2ccccc2C(=O)O)c2cc(-c3ccc(OC(F)F)nc3)n(C)c(=O)c2c1] C₂₆H₂₃F₂N₃O₄ | 189 | | ES− 478.1 [M − H] | ¹H NMR (400 MHz, Methanol-d₄) δ 8.42- 8.37 (m, 1H), 8.11- 8.01 (m, 2H), 7.92- 7.81 (m, 1H), 7.69- 7.42 (m, 2H), 7.16- 7.06 (m, 2H), 6.99 (s, 1H), 6.55-6.48 (m, 1H), 6.35-6.29 (m, 1H), 5.20-5.12 (m, 1H), 3.46-3.43 (m, 3H), 2.41 (s, 3H), 1.62-1.58 (m, 3H). |
| 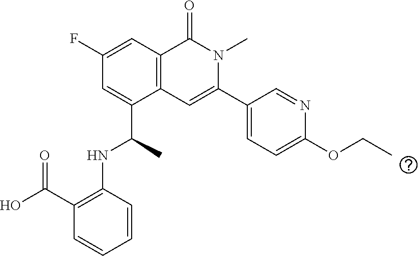 C₂₇H₂₄F₃N₃O₄ | 190 | | ES+ 512.3 [M + H] | ¹H NMR (400 MHz, Methanol-d₄) δ 8.38- 8.32 (m, 1H), 8.11- 8.06 (m, 1H), 7.97- 7.86 (m, 2H), 7.62- 7.59 (m, 1H), 7.15- 7.03 (m, 2H), 6.97 (s, 1H), 6.53-6.48 (m, 1H), 6.33-6.28 (m, 1H), 5.17-5.12 (m, 1H), 4.99-4.96 (m, 2H), 3.45 (s, 3H), 2.41 (s, 3H), 1.62-1.58 (m, 3H). |
| 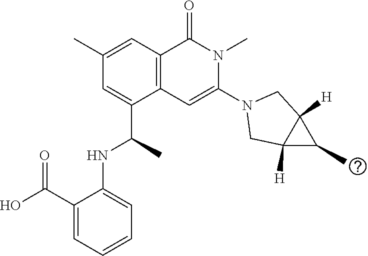 C₂₆H₂₆F₃N₃O₃ | 191 | | ES+ 486.2 [M + H] | ¹H NMR (400 MHz, DMSO-d₆) δ 12.92 (s, 1H), 8.56 (s, 1H), 7.87-7.77 (m, 2H), 7.41 (d, J = 1.9 Hz, 1H), 7.15 (m, 1H), 6.54-6.44 (m, 2H), 6.33 (d, J = 8.5 Hz, 1H), 5.18-5.12 (m, 1H), 3.50-3.43 (m, 5H), 3.22 (d, J = 9.4 Hz, 1H), 3.16 (d, J = 9.4 Hz, 1H), 2.31 (s, 3H), 2.24-2.22 (m, 1H), 2.12 (d, J = 16 Hz, 2H), 1.51 (d, J = 6.5 Hz, 3H). |
| 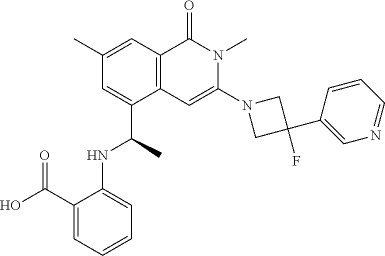 [SMILES: Cc1cc([C@@H](C)Nc2ccccc2C(=O)O)c2cc(N3CC(F)(c4cccnc4)C3)n(C)c(=O)c2c1] C₂₈H₂₇FN₄O₃ | 192 | | ES− 485.3 [M − H] | ¹H NMR (400 MHz, DMSO-d₆) δ 8.85 (d, J = 2.3 Hz, 1H), 8.69- 8.63 (m, 1H), 8.56 (s, 1H), 8.03 (d, J = 8.1 Hz, 1H), 7.86- 7.77 (m, 2H), 7.55- 7.52 (m, 1H), 7.39 (d, J = 1.9 Hz, 1H), 7.16-7.11 (m, 1H), 6.50-6.47 (m, 1H), 6.33 (d, J = 8.5 Hz, 1H), 6.09 (s, 1H), 5.16-5.12 (m, 1H), 4.63-4.41 (m, 4H), 3.47 (s, 3H), 2.30 (s, 3H), 1.52 (d, J = 6.5 Hz, 3H). |
| 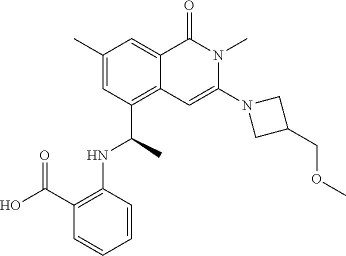 [SMILES: COCC1CN(c2cc3c([C@@H](C)Nc4ccccc4C(=O)O)cc(C)cc3c(=O)n2C)C1] C₂₅H₂₉N₃O₄ | 193 | | ES− 436.3 [M + H] | ¹H NMR (400 MHz, DMSO-d₆) δ 12.77 (s, 1H), 8.45 (s, 1H), 7.80 (d, J = 6.7 Hz, 2H), 7.35 (d, J = 2.0 Hz, 1H), 7.14 (s, 1H), 6.52-6.47 (m, 1H), 6.31 (d, J = 8.5 Hz, 1H), 5.83 (s, 1H), 5.10-5.05 (m, 1H), 4.05-4.01 (m, 2H), 3.69-3.65 (m, 2H), 3.55 (d, J = 6.6 Hz, 2H), 3.40 (s, 3H), 3.29 (s, 3H), 2.88-2.83 (m, 1H), 2.28 (s, 3H), 1.52 (d, J = 6.5 Hz, 3H). |
| 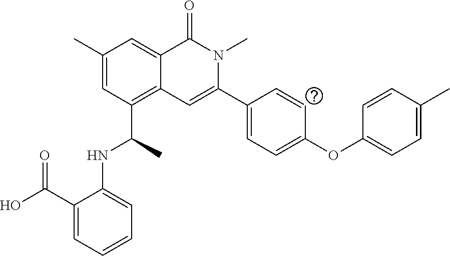 [SMILES: Cc1ccc(Oc2ccc(-c3cc4c([C@@H](C)Nc5ccccc5C(=O)O)cc(C)cc4c(=O)n3C)cc2)cc1] C₃₁H₂₆FN₃O₄ | 194 | | ES− 522.2 [M − H] | ¹H NMR (400 MHz, DMSO-d₆) δ 8.74 (s, 1H), 8.28 (s, 1H), 8.16-8.11 (m, 1H), 7.98 (s, 1H), 7.87 (d, J = 2.1 Hz, 1H), 7.81 (d, J = 7.8 Hz, 1H), 7.50 (d, J = 1.9 Hz, 1H), 7.33-7.17 (m, 5H), 6.92 (s, 1H), 6.48 (d, J = 7.5 Hz, 1H), 6.29 (d, J = 8.5 Hz, 1H), 5.20-5.14 (m, 1H), 3.38-3.25 (m, 3H), 2.36 (s, 3H), 1.50-1.43 (m, 3H). |
| 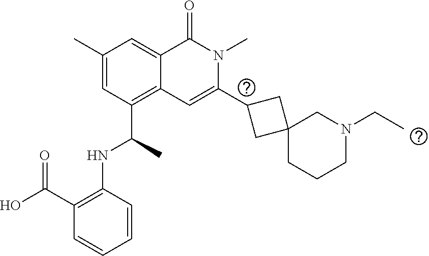 C₂₉H₃₃F₃N₄O₃ | 195 | | ES+ 543.3 [M + H] | ¹H NMR (400 MHz, DMSO-d₆) δ 8.60 (s, 1H), 7.82-7.60 (m, 2H), 7.35 (d, J = 1.9 Hz, 1H), 7.19-7.05 (m, 1H), 6.55-6.40 (m, 1H), 6.32 (d, J = 8.5 Hz, 1H), 5.83 (s, 1H), 5.12-5.05 (m, 1H), 3.69-3.63 (m, 4H), 3.40 (s, 3H), 3.26-3.18 (m, 2H), 2.79 (s, 2H), 2.58 (d, J = 5.5 Hz, 2H), 2.28 (s, 3H), 1.64-1.56 (m, 2H), 1.55-1.50 (m, 5H). |
| 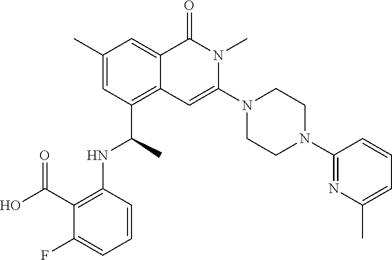 [SMILES: Cc1cc([C@@H](C)Nc2cccc(F)c2C(=O)O)c2cc(N3CCN(c4cccc(C)n4)CC3)n(C)c(=O)c2c1] C₃₀H₃₃N₅O₃ | 196 | | ES+ 512.4 [M + H] | ¹H NMR (400 MHz, Methanol-d₄) δ 8.07- 7.93 (m, 1H), 7.92- 7.81 (m, 1H), 7.54 (d, J = 1.8 Hz, 1H), 7.50-7.42 (m, 1H), 7.15-7.03 (m, 1H), 6.65-6.56 (m, 2H), 6.53 (s, 1H), 6.52- 6.45 (m, 1H), 6.36 (d, J = 8.4 Hz, 1H), 5.16-5.00 (m, 1H), 4.26 (s, 2H), 3.67 (s, 3H), 3.29-2.65 (m, 6H), 2.37 (d, J = 4.5 Hz, 6H), 1.63 (d, J = 6.7 Hz, 3H). |
| 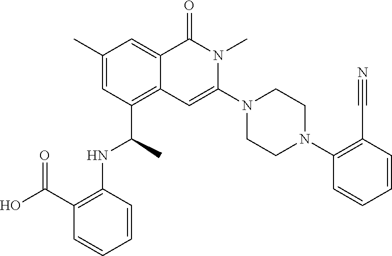 [SMILES: Cc1cc([C@@H](C)Nc2ccccc2C(=O)O)c2cc(N3CCN(c4ccccc4C#N)CC3)n(C)c(=O)c2c1] C₃₁H₃₁N₅O₃ | 197 | | ES− 520.3 [M − H] | ¹H NMR (400 MHz, DMSO-d₆) δ 8.91 (s, 1H), 7.88 (d, J = 1.9 Hz, 1H), 7.80-7.75 (m, 2H), 7.68-7.64 (m, 1H), 7.43 (d, J = 1.9 Hz, 1H), 7.27 (d, J = 8.3 Hz, 1H), 7.17- 7.07 (m, 2H), 6.50- 6.44 (m, 2H), 6.30 (d, J = 8.4 Hz, 1H), 5.21-5.17 (m, 1H), 3.56 (s, 3H), 3.30- 2.93 (m, 8H), 2.32 (s, 3H), 1.53-1.52 (d, J = 6.6 Hz, 3H). |
| 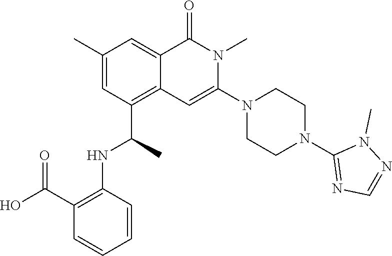 [SMILES: Cc1cc([C@@H](C)Nc2ccccc2C(=O)O)c2cc(N3CCN(c4ncnn4C)CC3)n(C)c(=O)c2c1] C₂₇H₃₁N₇O₃ | 198 | | ES− 500.3 [M − H] | ¹H NMR (400 MHz, DMSO-d₆) δ 12.68 (s, 1H), 8.41 (d, J = 5.9 Hz, 1H), 7.90 (d, J = 1.8 Hz, 1H), 7.81- 7.78 (m, 1H), 7.66 (s, 1H), 7.43 (d, J = 1.9 Hz, 1H), 7.24- 7.18 (m, 1H), 6.55- 6.51 (m, 1H), 6.46 (s, 1H), 6.39 (d, J = 8.5 Hz, 1H), 5.22- 5.17 (m, 1H), 3.71 (s, 3H), 3.55 (s, 3H), 3.29-2.98 (m, 8H) 2.34 (s, 3H), 1.55 (d, J = 6.5 Hz, 3H). |
| 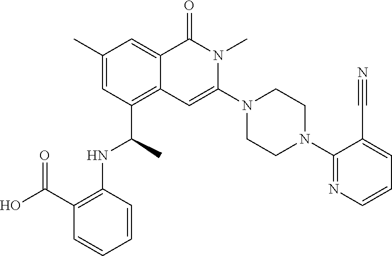 [SMILES: Cc1cc([C@@H](C)Nc2ccccc2C(=O)O)c2cc(N3CCN(c4ncccc4C#N)CC3)n(C)c(=O)c2c1] C₃₀H₃₀N₆O₃ | 199 | | ES+ 523.3 [M + H] | ¹H NMR (400 MHz, DMSO-d₆, ppm) 8 9.03 (s, 1H), 8.47- 8.45 (m, 1H), 8.15- 8.12 (m, 1H), 7.87 (s, 1H), 7.79 (d, J = 7.7 Hz, 1H), 7.43 (d, J = 2.0 Hz, 1H), 7.04- 6.80 (m, 2H), 6.47- 6.39 (m, 2H), 6.27 (d, J = 8.4 Hz, 1H), 5.17-5.11 (m, 1H), 4.65-3.98 (m, 2H), 3.57 (s, 3H), 3.29- 2.79 (m, 6H), 2.31 (s, 3H), 1.50 (d, J = 6.5 Hz, 3H). |
| 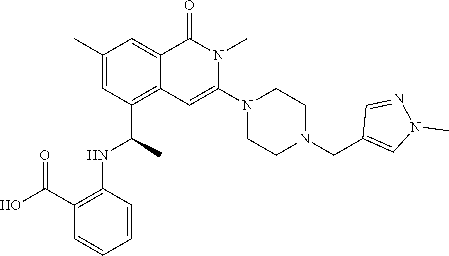 [SMILES: Cc1cc([C@@H](C)Nc2ccccc2C(=O)O)c2cc(N3CCN(Cc4cnn(C)c4)CC3)n(C)c(=O)c2c1] C₂₉H₃₄N₆O₃ | 200 | | ES+ 515.3 [M + H] | ¹H NMR (400 MHz, DMSO-d₆) δ 8.38 (s, 1H), 7.87 (s, 1H), 7.82-7.78 (m, 1H), 7.59 (s, 1H), 7.45- 7.40 (m, 1H), 7.33 (s, 1H), 7.24-7.14 (m, 1H), 6.53-6.50 (m, 1H), 6.43-6.33 (m, 2H), 5.15-5.10 (m, 1H), 3.80 (s, 3H), 3.47 (s, 3H), 3.43 (s, 2H), 3.23- 2.85 (m, 8H), 2.32 (s, 3H), 1.52-1.50 (d, J = 7.5 Hz, 3H). |
| 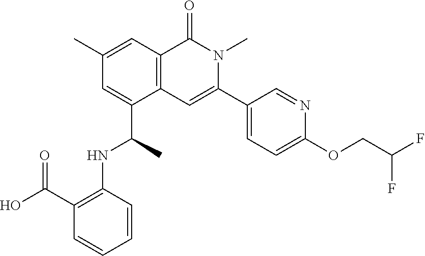 [SMILES: Cc1cc([C@@H](C)Nc2ccccc2C(=O)O)c2cc(-c3ccc(OCC(F)F)nc3)n(C)c(=O)c2c1] C₂₇H₂₅F₂N₃O₄ | 201 | | ES+ 494.2 [M + H] | ¹H NMR (400 MHz, Methanol-d₄) δ 8.36- 8.32 (m, 1H), 8.12- 8.06 (m, 1H), 7.92- 7.90 (m, 2H), 7.62- 7.58 (m, 1H), 7.13- 7.08 (m, 1H), 7.05- 6.93 (m, 2H), 6.56- 6.47 (m, 1H), 6.41- 6.08 (m, 2H), 5.15- 5.09 (m, 1H), 4.64- 4.60 (m, 2H), 3.46 (s, 3H), 2.42 (s, 3H), 1.62-1.58 (m, 3H). |
| 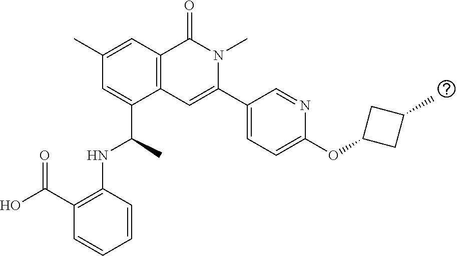 [SMILES: Cc1cc([C@@H](C)Nc2ccccc2C(=O)O)c2cc(-c3ccc(O[C@H]4C[C@@H](C)C4)nc3)n(C)c(=O)c2c1] C₂₉H₂₈FN₃O₄ | 202 | | ES− 500.1 [M − H] | ¹H NMR (400 MHz, Methanol-d₄) δ 8.32- 8.27 (m, 1H), 8.08 (s, 1H), 7.95-7.73 (m, 2H), 7.61-7.57 (m, 1H), 7.13-7.10 (m, 1H), 6.97-6.88 (m, 2H), 6.54-6.48 (m, 1H), 6.33-6.30 (m, 1H), 5.18-5.13 (m, 1H), 4.96-4.92 (m, 1H), 4.76-4.72 (m, 1H), 3.46 (s, 3H), 3.14-2.94 (m, 2H), 2.45-2.23 (m, 5H), 1.62-1.59 (m, 3H). |
| 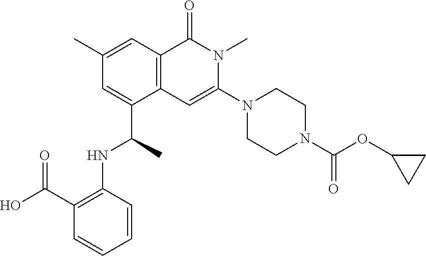 [SMILES: Cc1cc([C@@H](C)Nc2ccccc2C(=O)O)c2cc(N3CCN(C(=O)OC4CC4)CC3)n(C)c(=O)c2c1] C₂₈H₃₂N₄O₅ | 203 | | ES+ 505.3 [M + H] | ¹H NMR (400 MHz, Methanol-d₄) δ 7.97 (s, 1H), 7.89-7.88 (m, 1H), 7.55 (d, J = 1.9 Hz, 1H), 7.14- 7.06 (m, 1H), 6.55- 6.46 (m, 2H), 6.35 (d, J = 8.4 Hz, 1H), 5.09-5.06 (m, 1H), 4.33-3.90 (m, 3H), 3.64 (s, 3H), 3.16 (s, 4H), 2.73 (s, 2H), 2.36 (s, 3H), 1.62 (d, J = 6.7 Hz, 3H), 0.75- 0.68 (m, 4H). |
| 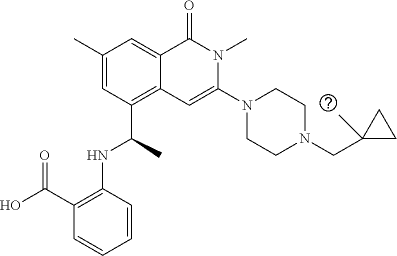 C₂₈H₃₃FN₄O₃ | 204 | | ES+ 493.3 [M + H] | ¹H NMR (400 MHz, DMSO-d₆) δ 8.48 (s, 1H), 7.87 (d, J = 1.9 Hz, 1H), 7.83-7.76 (m, 1H), 7.42 (d, J = 1.9 Hz, 1H), 7.22- 7.13 (m, 1H), 6.55- 6.47 (m, 1H), 6.42- 6.35 (m, 2H), 5.16- 5.06 (m, 1H), 3.50 (s, 3H), 3.38-2.99 (m, 8H), 2.83-2.77 (m, 2H), 2.32 (s, 3H), 1.54 (d, J = 6.5 Hz, 3H), 1.08-0.95 (m, 2H), 0.76-0.64 (m, 2H). |
| 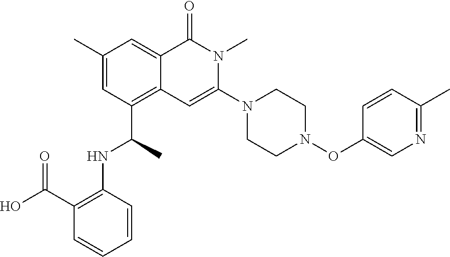 [SMILES: Cc1cc([C@@H](C)Nc2ccccc2C(=O)O)c2cc(N3CCN(Oc4ccc(C)nc4)CC3)n(C)c(=O)c2c1] C₃₁H₃₅N₅O₃ | 205 | | ES− 524.4 [M − H] | ¹H NMR (400 MHz, Methanol-d₄) δ 8.40 (d, J = 2.2 Hz, 1H), 7.96 (s, 1H), 7.88- 7.77 (m, 1H), 7.77- 7.75 (m, 1H), 7.53 (s, 1H), 7.31 (d, J = 7.9 Hz, 1H), 7.11- 7.09 (m, 1H), 6.54- 6.46 (m, 2H), 6.36 (d, J = 8.5 Hz, 1H), 5.08-5.05 (m, 1H), 3.63 (d, J = 14.1 Hz, 5H), 3.20-2.70 (m, 6H), 2.53-2.37 (m, 8H), 1.62 (d, J = 6.7 Hz, 3H). |
| 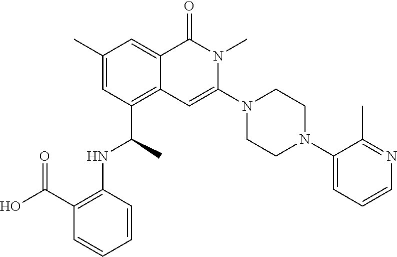 [SMILES: Cc1cc([C@@H](C)Nc2ccccc2C(=O)O)c2cc(N3CCN(c4cccnc4C)CC3)n(C)c(=O)c2c1] C₃₀H3N₅O₃ | 206 | | ES+ 512.6 [M + H] | ¹H NMR (400 MHz, DMSO-d₆) δ 12.77 (s, 1H), 8.38 (d, J = 6.0 Hz, 1H), 8.17 (d, J = 4.7 Hz, 1H), 7.89 (s, 1H), 7.81 (d, J = 7.9 Hz, 1H), 7.50 (d, J = 8.0 Hz, 1H), 7.43 (s, 1H), 7.23-7.21 (m, 2H), 6.55-6.52 (m, 1H), 6.48 (s, 1H), 6.42 (d, J = 8.5 Hz, 1H), 5.20-5.12 (m, 1H), 3.56 (s, 3H), 3.10-3.05 (m, 8H), 2.51 (s, 3H), 2.33 (s, 3H), 1.56 (d, J = 6.5 Hz, 3H). |
| 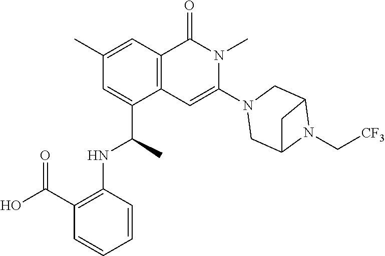 [SMILES: Cc1cc([C@@H](C)Nc2ccccc2C(=O)O)c2cc(N3CC4CC(C3)N4CC(F)(F)F)n(C)c(=O)c2c1] C₂₇H₂₉F₃N₄O₃ | 207 | | ES+ 515.1 [M + H] | ¹H NMR (400 MHz, DMSO-d₆) δ 12.70 (s, 1H), 8.47 (s, 1H), 7.88 (d, J = 1.9 Hz, 1H), 7.81-7.79 (m, 1H), 7.43 (d, J = 1.9 Hz, 1H), 7.21-7.16 (m, 1H), 6.77 (s, 1H), 6.53-6.49 (m, 1H), 6.41 (d, J = 8.5 Hz, 1H), 5.28-5.25 (m, 1H), 3.67-3.57 (m, 4H), 3.50 (s, 3H), 3.34 (d, J =3.4 Hz, 2H), 3.40-3.19 (m, 2H), 2.58-2.54 (m, 1H), 2.33 (s, 3H), 1.91 (d, J = 8.2 Hz, 1H), 1.53 (d, J = 6.5 Hz, 3H). |
| 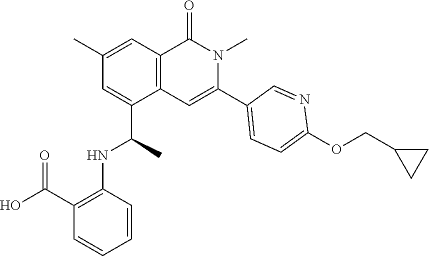 [SMILES: Cc1cc([C@@H](C)Nc2ccccc2C(=O)O)c2cc(-c3ccc(OCC4CC4)nc3)n(C)c(=O)c2c1] C₂₉H₂₉N₃O₄ | 208 | | ES− 482.3 [M − H] | ¹H NMR (400 MHz, DMSO-d₆) δ 12.78 (s, 1H), 8.58-8.32 (m, 2H), 8.01-7.93 (m, 2H), 7.86-7.76 (m, 1H), 7.49 (d, J = 1.9 Hz, 1H), 7.18- 7.09 (m, 1H), 6.97 (d, J = 8.6 Hz, 1H), 6.89 (s, 1H), 6.53- 6.46 (m, 1H), 6.32 (d, J = 8.5 Hz, 1H), 5.21-5.15 (m, 1H), 4.17 (d, J = 7.2 Hz, 2H), 3.35 (s, 3H), 2.37 (s, 3H), 1.47- 1.44 (d, J = 6.8 Hz, 3H), 1.35-1.21 (m, 1H), 0.63-0.52 (m, 2H), 0.40-0.31 (m, 2H). |
| 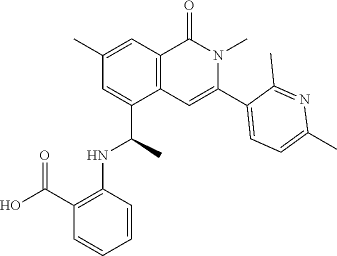 [SMILES: Cc1cc([C@@H](C)Nc2ccccc2C(=O)O)c2cc(-c3ccc(C)nc3C)n(C)c(=O)c2c1] C₂₇H₂₇N₃O₃ | 209 | | ES+ 442.2 [M + H] | ¹H NMR (400 MHz, DMSO-d₆) δ 8.00 (s, 1H), 7.82-7.79 (m, 1H), 7.73-7.70 (m, 1H), 7.53-7.49 (m, 1H), 7.27-7.24 (m, 1H), 7.16-7.06 (m, 1H), 6.85 (d, J = 12.8 Hz, 1H), 6.50- 6.47 (m, 1H), 6.34 (d, J = 8.5 Hz, 1H), 6.27 (d, J = 8.4 Hz, 1H), 5.17-5.14 (m, 1H), 3.19 (s, 3H), 2.52 (d, J = 1.2 Hz, 3H), 2.40-2.31 (m, 6H), 1.49-1.48 (d, J = 2.4 Hz, 3H). |
| 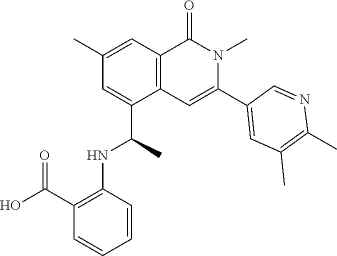 [SMILES: Cc1cc([C@@H](C)Nc2ccccc2C(=O)O)c2cc(-c3cnc(C)c(C)c3)n(C)c(=O)c2c1] C₂₇H₂₇N₃O₃ | 210 | | ES+ 442.1 [M + H] | ¹H NMR (400 MHz, Methanol-d₄) δ 8.40 (s, 1H), 8.08 (s, 1H), 7.89 (d, J = 8.0 Hz, 1H), 7.76 (s, 1H), 7.60 (s, 1H), 7.21- 7.11 (m, 1H), 6.95 (s, 1H), 6.54-6.48 (m, 1H), 6.32 (d, J = 8.5 Hz, 1H), 5.15- 5.10 (m, 1H), 3.44 (s, 3H), 2.58 (s, 3H), 2.42 (d, J = 2.7 Hz, 6H), 1.60 (d, J = 6.6 Hz, 3H). |
| 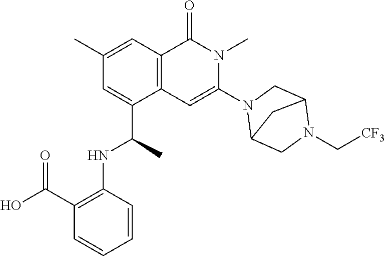 [SMILES: Cc1cc([C@@H](C)Nc2ccccc2C(=O)O)c2cc(N3CC4CC3CN4CC(F)(F)F)n(C)c(=O)c2c1] C₂₇H₂₉F₃N₄O₃ | 211 | diastereomer 1 | ES+ 515.4 [M + H] | ¹H NMR (400 MHz, Methanol-d₄) δ 7.97- 7.83 (m, 2H), 7.51 (d, J = 1.9 Hz, 1H), 7.20-7.06 (m, 1H), 6.55-6.47 (m, 1H), 6.43 (d, J = 8.4 Hz, 1H), 5.10-5.00 (m, 1H), 4.01 (s, 1H), 3.66 (s, 1H), 3.60 (s, 3H), 3.47 (d, J = 9.8 Hz, 1H), 3.27 (d, J = 9.6 Hz, 2H), 3.20- 3.12 (m, 1H), 3.11- 3.03 (m, 1H), 2.97 (d, J = 10.0 Hz, 1H), 2.36 (s, 3H), 1.87 (d, J = 3.4 Hz, 2H), 1.63 (d, J = 6.7 Hz, 3H). |
| 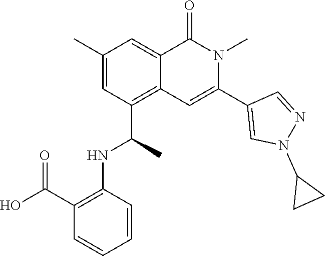 [SMILES: Cc1cc([C@@H](C)Nc2ccccc2C(=O)O)c2cc(-c3cnn(C4CC4)c3)n(C)c(=O)c2c1] C₂₆H₂₆N₄O₃ | 212 | | ES− 441.2 [M − H] | ¹H NMR (400 MHz, DMSO-d₆) δ 12.78 (s, 1H), 8.47 (s, 1H), 8.23 (s, 1H), 7.94 (s, 1H), 7.79 (d, J = 5.1 Hz, 2H), 7.44 (d, J = 1.9 Hz, 1H), 7.18- 7.14 (m, 1H), 6.89 (s, 1H), 6.53-6.49 (m, 1H), 6.32 (d, J = 8.5 Hz, 1H), 5.21- 5.17 (m, 1H), 3.83- 3.77 (m, 1H), 3.53 (s, 3H), 2.35 (s, 3H), 1.51 (d, J = 6.5 Hz, 3H), 1.17-1.11 (m, 2H), 1.03-1.01 (m, 2H). |
| 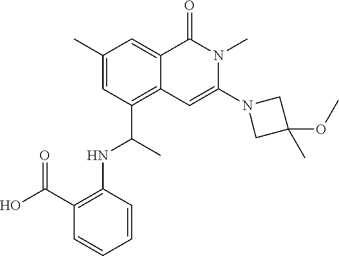 [SMILES: COC1(C)CN(c2cc3c(C(C)Nc4ccccc4C(=O)O)cc(C)cc3c(=O)n2C)C1] C₂₅H₂₉N₃O₄ | 213 | enantiomer 2 | ES− 434.3 [M − H] | ¹H NMR (400 MHz, DMSO-d₆) δ 8.89 (s, 1H), 7.79 (s, 2H), 7.38 (d, J = 1.9 Hz, 1H), 7.13-7.04 (m, 1H), 6.48-6.44 (m, 1H), 6.29 (d, J = 8.4 Hz, 1H), 5.90 (s, 1H), 5.10-5.05 (m, 1H), 3.87-3.73 (m, 4H), 3.41 (s, 3H), 3.21 (s, 3H), 2.28 (s, 3H), 1.51 (d, J = 4.8 Hz, 6H). |
| 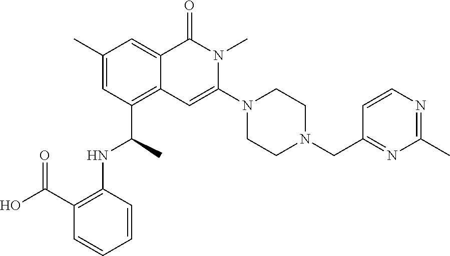 [SMILES: Cc1cc([C@@H](C)Nc2ccccc2C(=O)O)c2cc(N3CCN(Cc4ccnc(C)n4)CC3)n(C)c(=O)c2c1] C₃₀H₃₄N₆O₃ | 214 | | ES− 525.1 [M − H] | ¹H NMR (400 MHz, Methanol-d₄) δ 8.66- 8.62 (m, 1H), 7.97 (s, 1H), 7.91-7.88 (m, 1H), 7.58-7.46 (m, 2H), 7.16-7.12 (m, 1H), 6.56-6.45 (m, 2H), 6.40-6.37 (m, 1H), 5.12-5.09 (m, 1H), 3.74 (s, 2H), 3.63 (s, 3H), 3.13-2.80 (m, 8H), 2.69 (s, 3H), 2.59- 2.48 (m, 1H), 2.37 (s, 3H), 1.64-1.63 (d, J = 6.8 Hz, 3H). |
| 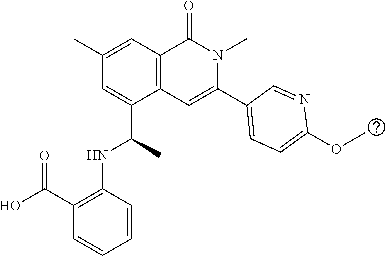 [SMILES: COc1ccc(-c2cc3c([C@@H](C)Nc4ccccc4C(=O)O)cc(C)cc3c(=O)n2C)cn1] C₂₆H₂₂F₃N₃O₄ | 215 | | ES− 496.0 [M − H] | ¹H NMR (400 MHz, DMSO-d₆) δ 8.66- 8.62 (m, 1H), 8.46 (s, 1H), 8.34-8.30 (m, 1H), 8.03-8.00 (m, 1H), 7.82-7.78 (m, 1H), 7.52-7.42 (m, 2H), 7.21-7.10 (m, 1H), 7.02 (s, 1H), 6.53-6.50 (m, 1H), 6.35-6.31 (m, 1H), 5.30-5.25 (m, 1H), 3.30 (s, 3H), 2.38 (s, 3H), 1.52- 1.49 (m, 3H). |
| 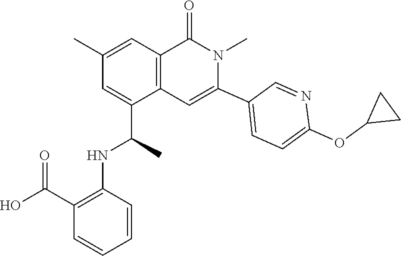 [SMILES: Cc1cc([C@@H](C)Nc2ccccc2C(=O)O)c2cc(-c3ccc(OC4CC4)nc3)n(C)c(=O)c2c1] C₂₈H₂₇N₃O₄ | 216 | | ES− 468.0 [M − H] | ¹H NMR (400 MHz, Methanol-d₄) δ 8.36- 3.33 (m, 1H), 8.10- 8.06 (m, 1H), 7.92- 7.88 (m, 2H), 7.61- 7.59 (m, 1H), 7.14- 7.04 (m, 2H), 6.96 (s, 1H), 6.53-6.50 (m, 1H), 6.34-6.30 (m, 1H), 5.17-5.13 (m, 1H), 4.27-4.18 (m, 1H), 3.46 (s, 3H), 2.42 (s, 3H), 1.63-1.59 (m, 3H), 0.91-0.64 (m, 4H). |
| 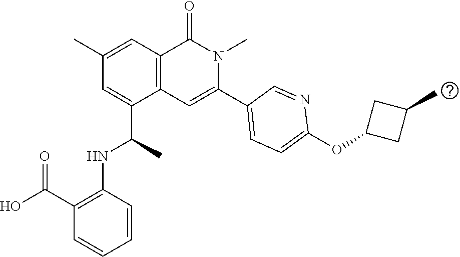 C₂₉H₂₈FN₃O₄ | 217 | | ES+ 502.3 [M + H] | ¹H NMR (400 MHz, Methanol-d₄) δ 8.32- 8.28 (m, 1H), 8.10- 8.05 (m, 1H), 7.93- 7.82 (m, 2H), 7.61- 7.58 (m, 1H), 7.13- 7.09 (m, 1H), 6.96- 6.89 (m, 2H), 6.53- 6.48 (m, 1H), 6.32- 6.28 (m, 1H), 5.53- 5.19 (m, 2H), 5.16- 5.12 (m, 1H), 3.45 (s, 3H), 2.81-2.64 (m, 2H), 2.68-2.49 (m, 2H), 2.41 (s, 3H), 1.61-1.59 (d, J = 6.8 Hz, 3H). |
| 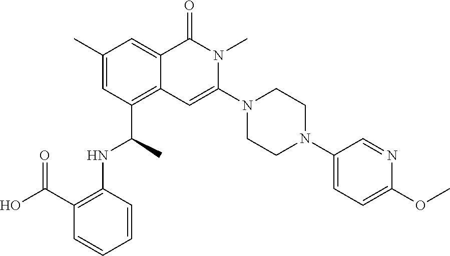 [SMILES: COc1ccc(N2CCN(c3cc4c([C@@H](C)Nc5ccccc5C(=O)O)cc(C)cc4c(=O)n3C)CC2)cn1] C₃₀H₃₃N₅O₄ | 218 | | ES+ 528.2 [M + H] | ¹H NMR (400 MHz, Methanol-d₄) δ 7.99 (s, 1H), 7.92-7.88 (m, 1H), 7.85-7.81 (m, 1H), 7.57-7.49 (m, 2H), 7.17-7.12 (m, 1H), 6.79-6.68 (m, 1H), 6.58 (s, 1H), 6.54-6.50 (m, 1H), 6.41-6.38 (m, 1H), 5.15-5.12 (m, 1H), 3.86 (s, 3H), 3.67 (s, 3H), 3.58- 3.46 (m, 2H), 3.21- 3.02 (m, 2H), 3.01- 2.99 (m, 4H), 2.42- 2.39 (m, 3H), 1.68- 1.65 (m, 3H). |
| 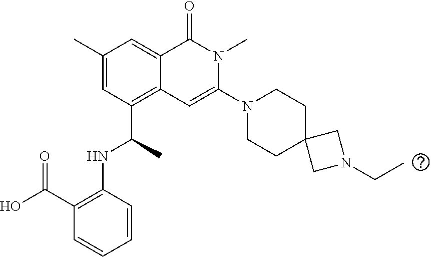 [SMILES: CCN1CC2(CCN(c3cc4c([C@@H](C)Nc5ccccc5C(=O)O)cc(C)cc4c(=O)n3C)CC2)C1] C₂₉H₃₃F₃N₄O₃ | 219 | | ES− 541.9 [M − H] | ¹H NMR (DMSO-d₆, 400 MHz): δ 8.80- 8.40 (s, 1H), 7.86 (s, 1H), 7.84-7.78 (m, 1H), 7.41 (d, J = 1.8 Hz, 1H), 7.21-7.12 (m, 1H), 6.55-6.47 (m, 1H), 6.35-6.28 (m, 2H), 5.15-5.05 (m, 1H), 3.47 (s, 3H), 3.24-3.19 (m, 8H), 3.04-2.98 (m, 2H), 2.32 (s, 3H), 1.92-1.75 (m, 4H), 1.53 (d, J = 6.5 Hz, 3H). |
| 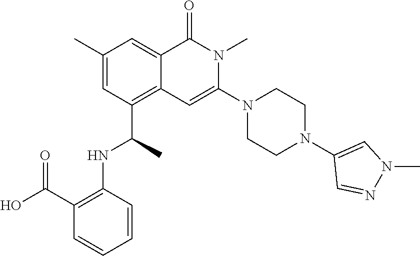 [SMILES: Cc1cc([C@@H](C)Nc2ccccc2C(=O)O)c2cc(N3CCN(c4cnn(C)c4)CC3)n(C)c(=O)c2c1] C₂₈H₃₂N₆O₃ | 220 | | ES− 499.3 [M − H] | ¹H NMR (400 MHz, DMSO-d₆) δ 12.50 (s, 1H), 8.48 (s, 1H), 7.88 (d, J = 1.9 Hz, 1H), 7.83-7.78 (m, 1H), 7.44 (d, J = 2.0 Hz, 1H), 7.32 (d, J = 0.9 Hz, 1H), 7.23- 7.14 (m, 2H), 6.56- 6.47 (m, 1H), 6.44- 6.36 (m, 2H), 5.18- 5.05 (m, 1H), 3.75 (s, 3H), 3.52 (s, 3H), 3.33-2.89 (m, 8H), 2.33 (s, 3H), 1.55 (d, J = 6.5 Hz, 3H). |
| 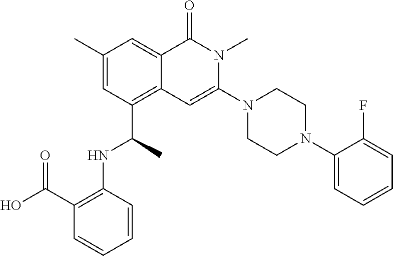 [SMILES: Cc1cc([C@@H](C)Nc2ccccc2C(=O)O)c2cc(N3CCN(c4ccccc4F)CC3)n(C)c(=O)c2c1] C₃₀H₃₁FN₄O₃ | 221 | | ES+ 515.3 [M + H] | ¹H NMR (400 MHz, DMSO-d₆) δ 8.64 (s, 1H), 7.88 (s, 1H), 7.81-7.79 (m, 1H), 7.43 (d, J = 1.9 Hz, 1H), 7.20-7.10 (m, 4H), 7.04-6.99 (m, 1H), 6.51-6.48 (m, 2H), 6.36 (d, J = 8.4 Hz, 1H), 5.23-5.17 (m, 1H), 3.55 (s, 3H), 3.16-2.67 (m, 8H), 2.33 (s, 3H), 1.53 (d, J = 6.6 Hz, 3H). |
| 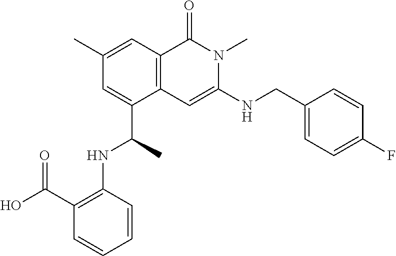 [SMILES: Cc1cc([C@@H](C)Nc2ccccc2C(=O)O)c2cc(NCc3ccc(F)cc3)n(C)c(=O)c2c1] C₂₇H₂₆FN₃O₃ | 222 | | ES− 458.2 [M − H] | ¹H NMR (400 MHz, Methanol-d₄) δ 7.91- 7.84 (m, 1H), 7.82 (d, J = 1.6 Hz, 1H), 7.49-7.41 (m, 2H), 7.32 (d, J = 1.9 Hz, 1H), 7.11-7.00 (m, 3H), 6.54-6.45 (m, 1H), 6.13 (d, J = 8.5 Hz, 1H), 5.41-5.26 (m, 1H), 4.75-4.68 (m, 1H), 4.51-4.38 (m, 2H), 3.66 (s, 3H), 2.27 (s, 3H), 1.36-1.26 (m, 3H). |
| 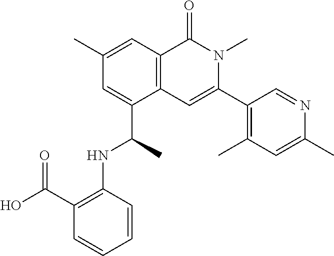 [SMILES: Cc1cc([C@@H](C)Nc2ccccc2C(=O)O)c2cc(-c3cnc(C)cc3C)n(C)c(=O)c2c1] C₂₇H₂₇N₃O₃ | 223 | | ES− 440.3 [M − H] | ¹H NMR (400 MHz, Methanol-d₄) δ 8.39 (d, J = 20.7 Hz, 1H), 8.13-8.08 (m, 1H), 7.95-7.88 (m, 1H), 7.69-7.62 (m, 1H), 7.37 (s, 1H), 7.12- 7.01 (m, 1H), 6.96 (d, J = 7.0 Hz, 1H), 6.55-6.45 (m, 1H), 6.34-6.25 (m, 1H), 5.17-5.06 (m, 1H), 4.87 (s, 3H), 2.61 (s, 3H), 2.42 (d, J = 4.4 Hz, 3H), 2.26 (d, J = 18.2 Hz, 3H), 1.65- 1.54 (m, 3H). |
| 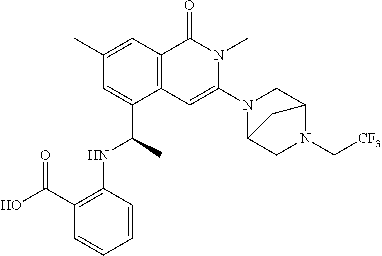 [SMILES: Cc1cc([C@@H](C)Nc2ccccc2C(=O)O)c2cc(N3CC4CC3CN4CC(F)(F)F)n(C)c(=O)c2c1] C₂₇H₂₉F₃N₄O₃ | 224 | diastereomer 2 | ES+ 515.2 [M + H] | ¹H NMR (400 MHz, DMSO-d₆) δ 8.49 (s, 1H), 7.95-7.86 (m, 2H), 7.39 (d, J = 1.9 Hz, 1H), 7.16-7.14 (m, 1H), 6.58-6.47 (m, 1H), 6.46-6.42 (m, 1H), 6.19 (s, 1H), 5.16-5.12 (m, 1H), 4.09 (s, 1H), 3.62 (s, 1H), 3.47 (s, 3H), 3.45-3.30 (m, 3H), 3.17 (d, J = 9.6 Hz, 1H), 3.00-2.91 (m, 1H), 2.91-2.80 (m, 1H), 2.30 (s, 3H), 1.80 (s, 2H), 1.64 (d, J = 6.7 Hz, 3H). |
| 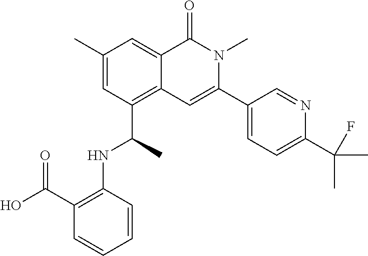 [SMILES: Cc1cc([C@@H](C)Nc2ccccc2C(=O)O)c2cc(-c3ccc(C(C)(C)F)nc3)n(C)c(=O)c2c1] C₂₈H₂₈FN₃O₃ | 225 | | ES+ 496.2 [M+Na] | ¹H NMR (400 MHz, DMSO-d₆) δ 12.75 (s, 1H), 8.81 (s, 1H), 8.38 (d, J = 5.9 Hz, 1H), 8.15-8.11 (m, 1H), 8.01 (s, 1H), 7.82-7.78 (m, 1H), 7.70 (d, J = 8.2 Hz, 1H), 7.49 (s, 1H), 7.19-7.15 (m, 1H), 6.98 (s, 1H), 6.55- 6.47 (m, 1H), 6.35 (d, J = 8.6 Hz, 1H), 5.30-5.23 (m, 1H), 3.36 (s, 3H), 2.38 (s, 3H), 1.76 (s, 3H), 1.70 (s, 3H), 1.50 (d, J = 6.5 Hz, 3H). |
| 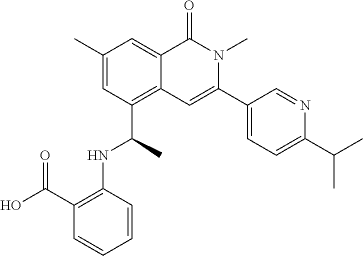 [SMILES: Cc1cc([C@@H](C)Nc2ccccc2C(=O)O)c2cc(-c3ccc(C(C)C)nc3)n(C)c(=O)c2c1] C₂₈H₂₉N₃O₃ | 226 | | ES+ 456.6 [M + H] | ¹H NMR (400 MHz, DMSO-d₆) δ 9.24 (s, 1H), 8.73 (d, J = 2.3 Hz, 1H), 8.02-7.95 (m, 2H), 7.80 (d, J = 7.6 Hz, 1H), 7.53 (d, J = 1.8 Hz, 1H), 7.44 (d, J = 8.1 Hz, 1H), 7.00-6.98 (m, 1H), 6.93 (s, 1H), 6.42- 6.40 (m, 1H), 6.21 (d, J = 8.3 Hz, 1H), 5.17-5.10 (br s, 1H), 3.33 (s, 3H), 3.15-3.12 (m, 1H), 2.36 (s, 3H), 1.47 (d, J = 6.5 Hz, 3H), 1.30 (d, J = 6.9 Hz, 6H). |
| 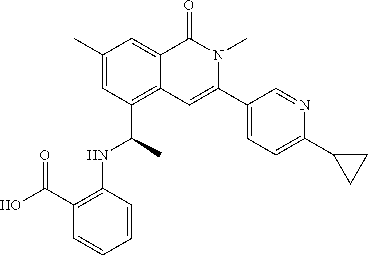 [SMILES: Cc1cc([C@@H](C)Nc2ccccc2C(=O)O)c2cc(-c3ccc(C4CC4)nc3)n(C)c(=O)c2c1] C₂₈H₂₇N₃O₃ | 227 | | ES+ 454.5 [M + H] | ¹H NMR (400 MHz, DMSO-d₆) δ 12.76 (s, 1H), 8.62 (d, J = 2.2 Hz, 1H), 8.42 (s, 1H), 7.99 (s, 1H), 7.92-7.89 (m, 1H), 7.81-7.79 (m, 1H), 7.51-7.42 (m, 2H), 7.20-7.11 (m, 1H), 6.90 (s, 1H), 6.53- 6.50 (m, 1H), 6.34 (d, J = 8.5 Hz, 1H), 5.25-5.22 (m, 1H), 3.34 (s, 3H), 2.37 (s, 3H), 2.23-2.20 (m, 1H), 1.50 (d, J = 6.5 Hz, 3H), 1.06-1.05 (m, 4H). |
| 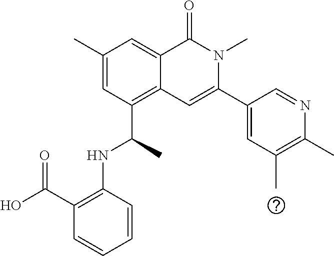 [SMILES: Cc1cc([C@@H](C)Nc2ccccc2C(=O)O)c2cc(-c3cnc(C)c(C)c3)n(C)c(=O)c2c1] C₂₆H₂₄FN₃O₃ | 228 | | ES− 444.2 [M − H] | ¹H NMR (400 MHz, Methanol-d₄) δ 8.48- 8.43 (m, 1H), 8.11- 8.06 (m, 1H), 7.92- 7.85 (m, 1H), 7.84- 7.77 (m, 1H), 7.62 (d, J = 1.9 Hz, 1H), 7.13-7.04 (m, 1H), 7.01 (s, 1H), 6.54- 6.46 (m, 1H), 6.31 (m, 1H), 5.20-5.11 (m, 1H), 3.46 (s, 3H), 2.60 (d, J = 2.9 Hz, 3H), 2.41 (s, 3H), 1.60 (d, J = 6.7 Hz, 3H). |
| 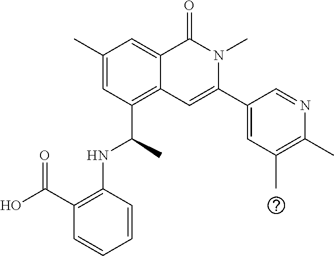 C₂₆H₂₄ClN₃O₃ | 229 | | ES+ 462.1 [M + H] | ¹H NMR (400 MHz, DMSO-d₆) δ 8.66 (d, J = 2.0 Hz, 1H), 8.51 (s, 1H), 8.22 (d, J = 2.0 Hz, 1H), 7.99 (s, 1H), 7.84-7.77 (m, 1H), 7.50 (d, J = 1.9 Hz, 1H), 7.12 (d, J = 7.7 Hz, 1H), 6.97 (s, 1H), 6.49 (d, J = 7.5 Hz, 1H), 6.31 (d, J = 8.5 Hz, 1H), 5.23- 5.20 (m, 1H), 3.36 (s, 3H), 2.64 (s, 3H), 2.37 (s, 3H), 1.49 (d, J = 6.5 Hz, 3H). |
| 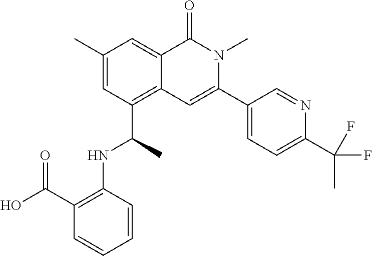 [SMILES: Cc1cc([C@@H](C)Nc2ccccc2C(=O)O)c2cc(-c3ccc(C(C)(F)F)nc3)n(C)c(=O)c2c1] C₂₇H₂₅F₂N₃O₃ | 230 | | ES+ 478.3 [M + H] | ¹H NMR (400 MHz, DMSO-d₆) δ 8.93 (d, J = 2.1 Hz, 1H), 8.65 (s, 1H), 8.29-8.26 (m,1H), 8.01 (s, 1H), 7.89-7.86 (m, 1H), 7.80 (d, J = 7.7 Hz, 1H), 7.51 (d, J = 1.9 Hz, 1H), 7.12-7.09 (m, 1H), 7.02 (s, 1H), 6.50-6.47 (m, 1H), 6.31 (d, J = 8.4 Hz, 1H), 5.27-5.20 (m, 1H), 3.36 (s, 3H), 2.38 (s, 3H), 2.12-2.03 (m, 3H), 1.49 (d, J = 6.5 Hz, 3H). |
| 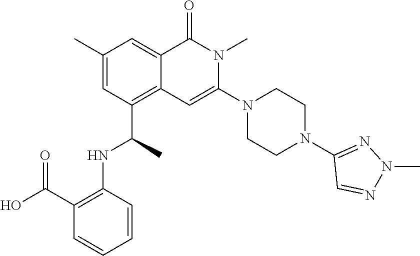 [SMILES: Cc1cc([C@@H](C)Nc2ccccc2C(=O)O)c2cc(N3CCN(c4cnn(C)n4)CC3)n(C)c(=O)c2c1] C₂₇H₃₁N₇O₃ | 231 | | ES− 500.1 [M − H] | ¹H NMR (300 MHz, DMSO-d₆) δ 12.74 (s, 1H), 8.40-8.36 (m, 1H), 7.91-7.86 (m, 1H), 7.82-7.79 (m, 1H), 7.45-7.41 (m, 1H), 7.33 (s, 1H), 7.20-7.19 (m, 1H), 6.55-6.50 (m, 1H), 6.43-6.39 (m, 2H), 5.26-5.05 (m, 1H), 3.99 (s, 3H), 3.53-3.52 (m, 2H), 3.25-2.89 (m, 6H), 2.33 (s, 3H), 2.08 (s, 3H), 1.57-1.53 (m, 3H). |
| 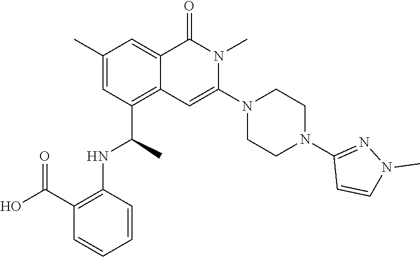 [SMILES: Cc1cc([C@@H](C)Nc2ccccc2C(=O)O)c2cc(N3CCN(c4ccn(C)n4)CC3)n(C)c(=O)c2c1] C₂₈H₃₂N₆O₃ | 232 | | ES− 499.0 [M − H] | ¹H NMR (400 MHz, Methanol-d₄) δ 7.98 (s, 1H), 7.92-7.88 (m, 1H), 7.56-7.53 (m, 1H), 7.42-7.36 (m, 1H), 7.16-7.12 (m, 1H), 6.57-6.48 (m, 2H), 6.41-6.36 (m, 1H), 5.80-5.75 (m, 1H), 5.15-5.10 (m, 1H), 3.74 (s, 3H), 3.68-3.66 (m, 4H), 3.33 (s, 3H), 3.05-2.89 (m, 4H), 2.38 (s, 3H), 1.66- 1.62 (m, 3H). |
| 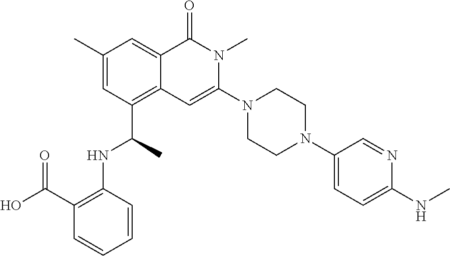 [SMILES: CNc1ccc(N2CCN(c3cc4c([C@@H](C)Nc5ccccc5C(=O)O)cc(C)cc4c(=O)n3C)CC2)cn1] C₃₀H₃₄N₆O₃ | 233 | | ES+ 527.1 [M + H] | ¹H NMR (400 MHz, Methanol-d₄) δ 8.02- 7.97 (m, 1H), 7.92- 7.88 (m, 1H), 7.62- 7.50 (m, 3H), 7.16- 7.12 (m, 1H), 6.72- 6.65 (m, 1H), 6.58 (s, 1H), 6.54-6.50 (m, 1H), 6.42-6.37 (m, 1H), 5.15-5.10 (m, 1H), 3.66 (s, 3H), 3.48-3.44 (m, 2H), 3.25-2.98 (m, 5H), 2.89-2.88 (m, 4H), 2.38 (s, 3H), 1.65 (d, J = 6.4 Hz, 3H) |
| 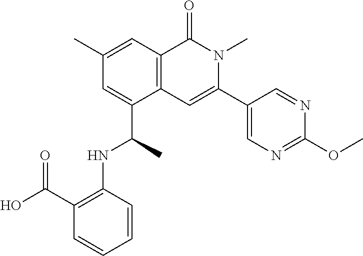 [SMILES: COc1ncc(-c2cc3c([C@@H](C)Nc4ccccc4C(=O)O)cc(C)cc3c(=O)n2C)cn1] C₂₅H₂₄N₄O₄ | 234 | | ES− 443.1 [M − H] | ¹H NMR (400 MHz, Methanol-d₄) δ 8.78 (s, 2H), 8.11-7.98 (m, 1H), 7.92-7.88 (m, 1H), 7.64-7.59 (m, 1H), 7.13-7.09 (m, 1H), 7.04 (s, 1H), 6.52-6.48 (m, 1H), 6.35-6.29 (m, 1H), 5.20-5.15 (m, 1H), 4.10 (s, 3H), 3.49 (s, 3H), 2.42 (s, 3H), 1.63-1.59 (m, 3H). |
| 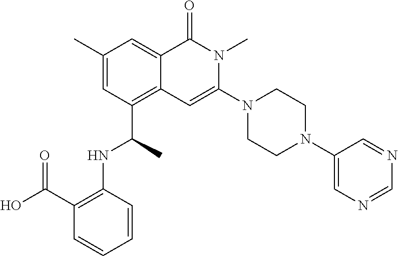 [SMILES: Cc1cc([C@@H](C)Nc2ccccc2C(=O)O)c2cc(N3CCN(c4cncnc4)CC3)n(C)c(=O)c2c1] C₂₈H₃₀N₆O₃ | 235 | | ES+ 499.6 [M + H] | ¹H NMR (400 MHz, DMSO-d₆) δ 12.72 (s, 1H), 8.64 (s, 1H), 8.58 (s, 2H), 8.38 (d, J = 6.1 Hz, 1H), 7.89 (s, 1H), 7.81-7.79 (m, 1H), 7.43 (d, J = 1.9 Hz, 1H), 7.21- 7.19 (m, 1H), 6.53- 6.51 (m, 1H), 6.45 (s, 1H), 6.40 (d, J = 8.5 Hz, 1H), 5.20- 5.18 (m, 1H), 4.12- 3.63 (m, 2H), 3.55 (s, 3H), 3.31-2.98 (m, 6H), 2.33 (s, 3H), 1.55 (d, J = 6.6 Hz, 3H). |
| 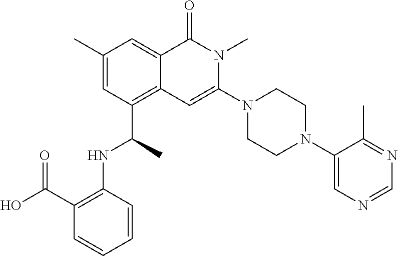 [SMILES: Cc1cc([C@@H](C)Nc2ccccc2C(=O)O)c2cc(N3CCN(c4cncnc4C)CC3)n(C)c(=O)c2c1] C₂₉H₃₂N₆O₃ | 236 | | ES+ 513.2 [M + H] | ¹H NMR (400 MHz, DMSO-d₆) δ 8.75 (s, 1H), 8.48 (s, 1H), 8.41 (d, J = 5.3 Hz, 1H), 7.89 (d, J = 1.9 Hz, 1H), 7.81-7.78 (m, 1H), 7.43 (d, J = 1.9 Hz, 1H), 7.22- 7.18 (m, 1H), 6.54- 6.50 (m, 1H), 6.48 (s, 1H), 6.41 (d, J = 8.5 Hz, 1H), 5.20- 5.15 (m, 1H), 3.56 (s, 3H), 3.32-3.17 (m, 8H), 2.65 (s, 3H), 2.33 (s, 3H), 1.56 (d, J = 6.5 Hz, 3H). |
| 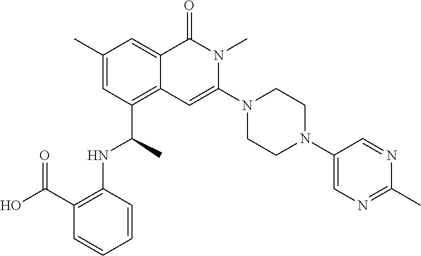 [SMILES: Cc1cc([C@@H](C)Nc2ccccc2C(=O)O)c2cc(N3CCN(c4cnc(C)nc4)CC3)n(C)c(=O)c2c1] C₂₉H₃₂N₆O₃ | 237 | | ES+ 513.6 [M + H] | ¹H NMR (400 MHz, DMSO-d₆) δ 12.71 (s, 1H), 8.47 (s, 2H), 8.38 (d, J = 6.1 Hz, 1H), 7.89 (s, 1H), 7.79-7.81 (m, 1H), 7.43 (d, J = 1.9 Hz, 1H), 7.22-7.19 (m, 1H), 6.53-6.50 (m, 1H), 6.45 (s, 1H), 6.40 (d, J = 8.5 Hz, 1H), 5.19-5.17 (m, 1H), 4.13-3.62 (m, 2H), 3.55 (s, 3H), 3.29-2.77 (s, 6H), 2.33 (s, 3H), 1.55 (d, J = 6.6 Hz, 3H), 1.41- 1.01 (m, 3H). |
| 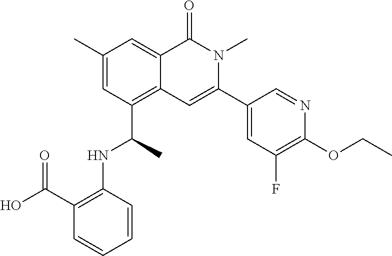 [SMILES: CCOc1ncc(-c2cc3c([C@@H](C)Nc4ccccc4C(=O)O)cc(C)cc3c(=O)n2C)cc1F] C₂₇H₂₆FN₃O₄ | 238 | | ES− 474.3 [M − H] | ¹H NMR (400 MHz, DMSO-d₆) δ 8.95 (s, 1H), 8.23 (d, J = 2.0 Hz, 1H), 8.08-8.05 (m, 1H), 7.97 (d, J = 1.6 Hz, 1H), 7.80- 7.78 (m, 1H), 7.50 (d, J = 1.9 Hz, 1H), 7.07-7.03 (m, 1H), 6.94 (s, 1H), 6.47- 6.43 (m, 1H), 6.25 (d, J = 8.5 Hz, 1H), 5.20-5.15 (m, 1H), 4.51-4.46 (m, 2H), 3.37-3.31 (m, 3H), 2.37 (s, 3H), 1.48 (d, J = 6.6 Hz, 3H), 1.42- 1.38 (m, 3H). |
| 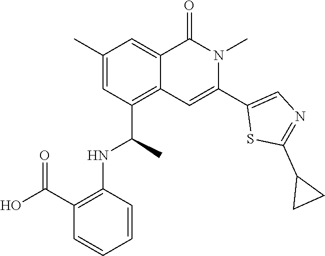 [SMILES: Cc1cc([C@@H](C)Nc2ccccc2C(=O)O)c2cc(-c3cnc(C4CC4)s3)n(C)c(=O)c2c1] C₂₆H₂₅N₃O₃S | 239 | | ES− 458.2 [M − H] | ¹H NMR (400 MHz, Methanol-d₄) δ 8.09 (d, J = 1.7 Hz, 1H), 7.91 (d, J = 1.7 Hz, 1H), 7.77 (s, 1H), 7.63 (d, J = 1.9 Hz, 1H), 7.13 (s, 1H), 7.11-7.07 (m,1H), 6.53-6.50 (m, 1H), 6.31 (d, J = 8.0 Hz, 1H), 5.17-5.10 (m, 1H), 4.59 (s, 1H), 3.57 (s, 3H), 2.50- 2.43 (m, 4H), 1.62 (d, J = 6.6 Hz, 3H), 1.26-1.18 (m, 2H), 1.15-1.13 (m, 2H). |
| 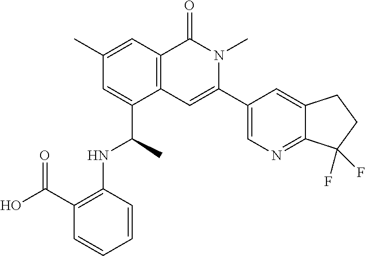 [SMILES: Cc1cc([C@@H](C)Nc2ccccc2C(=O)O)c2cc(-c3cnc4c(c3)CCC4(F)F)n(C)c(=O)c2c1] C₂₈H₂₅F₂N₃O₃ | 240 | | ES− 488.2 [M − H] | ¹H NMR (400 MHz, DMSO-d₆) δ 8.85 (d, J = 2.0 Hz, 1H), 8.69 (s, 1H), 8.17 (s, 1H), 8.01 (s, 1H), 7.81- 7.78 (m, 1H), 7.51 (d, J = 1.9 Hz, 1H), 7.11-7.08 (m, 1H), 7.00 (s, 1H), 6.49 (d, J = 7.5 Hz, 1H), 6.30 (d, J = 8.4 Hz, 1H), 5.21-5.15 (m, 1H), 3.36 (s, 3H), 3.18- 3.10 (m, 2H), 2.79- 2.71 (m, 2H), 2.38 (s, 3H), 1.49 (d, J = 6.5 Hz, 3H). |
| 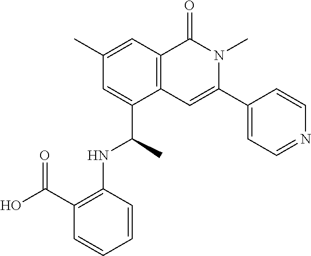 [SMILES: Cc1cc([C@@H](C)Nc2ccccc2C(=O)O)c2cc(-c3ccncc3)n(C)c(=O)c2c1] C₂₅H₂₃N₃O₃ | 241 | | ES+ 414.2 [M + H] | ¹H NMR (400 MHz, DMSO-d₆) δ 12.75 (s, 1H), 8.77-8.71 (m, 2H), 8.40 (d, J = 5.7 Hz, 1H), 8.04- 7.98 (m, 1H), 7.82- 7.77 (m, 1H), 7.67- 7.61 (m, 2H), 7.51 (d, J = 1.9 Hz, 1H), 7.19-7.10 (m, 1H), 6.95 (s, 1H), 6.55- 6.44 (m, 1H), 6.39- 6.32 (m, 1H), 5.29- 5.10 (m, 1H), 3.36 (s, 3H), 2.38 (s, 3H), 1.51 (d, J = 6.6 Hz, 3H). |
| 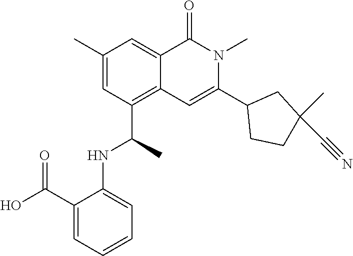 [SMILES: Cc1cc([C@@H](C)Nc2ccccc2C(=O)O)c2cc(C3CCC(C)(C#N)C3)n(C)c(=O)c2c1] C₂₆H₂₈N₄O₃ | 242 | pyrrolidine diastereomer | ES− 443.4 [M − H] | ¹H NMR (400 MHz, Methanol-d₄) δ 7.98 (s, 1H), 7.95-7.88 (m, 1H), 7.56 (d, J = 1.9 Hz, 1H), 7.21- 7.12 (m, 1H), 6.61- 6.49 (m, 2H), 6.42 (d, J = 8.5 Hz, 1H), 5.17-5.07 (m, 1H), 3.66 (s, 3H), 3.62- 3.46 (m, 2H), 3.29- 3.12 (m, 2H), 2.61- 2.45 (m, 1H), 2.38 (s, 3H), 2.19-2.09 (m, 1H), 1.66 (d, J = 6.7 Hz, 3H), 1.60 (s, 3H). |
| 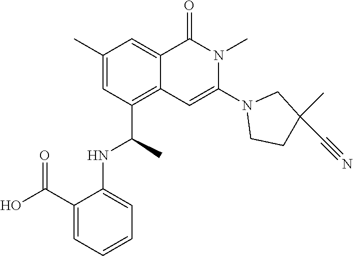 [SMILES: Cc1cc([C@@H](C)Nc2ccccc2C(=O)O)c2cc(N3CCC(C)(C#N)C3)n(C)c(=O)c2c1] C₂₆H₂₈N₄O₃ | 243 | pyrrolidine diastereomer | ES− 443.3 [M − H] | ¹H NMR (400 MHz, Methanol-d₄) δ 7.91 (s, 1H), 7.90-7.88 (m, 1H), 7.56 (d, J = 1.9 Hz, 1H), 7.15- 7.12 (m, 1H), 6.54- 6.50 (m, 2H), 6.38 (d, J = 8.5 Hz, 1H), 5.17-5.07 (m, 1H), 3.66 (s, 3H), 3.62- 3.46 (m, 2H), 3.29- 3.12 (m, 2H), 2.61- 2.45 (m, 1H), 2.37 (s, 3H), 2.19-2.09 (m, 1H), 1.62 (d, J = 6.7 Hz, 3H), 1.58 (s, 3H). |
| 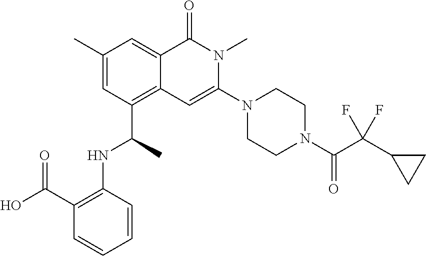 C₂₉H₃₄F₂N₄O₃ | 244 | | ES+ 525.4 [M + H] | ¹H NMR (400 MHz, DMSO-d₆) δ 8.74 (s, 1H), 7.86 (d, J = 1.9 Hz, 1H), 7.81 (d, J = 7.9 Hz, 1H), 7.43 (d, J = 1.9 Hz, 1H), 7.15- 7.07 (m, 1H), 6.51- 6.43 (m, 1H), 6.38 (s, 1H), 6.32 (d, J = 8.4 Hz, 1H), 5.12- 5.05 (m, 1H), 3.49 (s, 3H), 3.14-2.70 (m, 10H), 2.31 (s, 3H), 1.56-1.46 (m, 4H), 0.66-0.53 (m, 4H). |
| 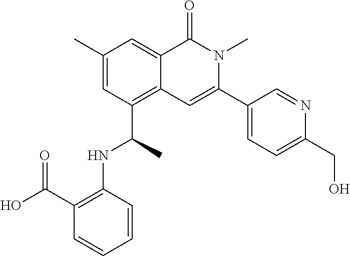 C₂₇H₂₄F₃N₃O₃ | 245 | | ES+ 496.5 [M + H] | ¹H NMR (400 MHz, DMSO-d₆) δ 12.75 (s, 1H), 8.85-8.82 (m, 1H), 8.40 (d, J = 5.9 Hz, 1H), 8.13- 8.11 (m, 1H), 8.03- 7.98 (m, 1H), 7.81- 7.79 (m, 1H), 7.73 (s, 1H), 7.64 (d, J = 8.0 Hz, 1H), 7.16- 7.19 (m, 1H), 6.99 (s, 1H), 6.53-6.51 (m, 1H), 6.37-6.33 (m, 1H), 5.31-5.23 (m, 1H), 3.97-3.91 (m, 2H), 3.35 (s, 3H), 2.38 (s, 3H), 1.50 (d, J = 6.6 Hz, 3H). |
| 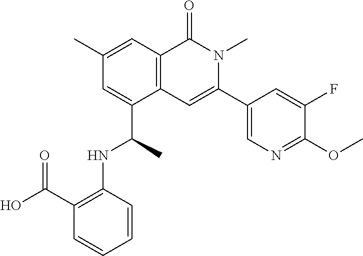 [SMILES: COc1ncc(-c2cc3c([C@@H](C)Nc4ccccc4C(=O)O)cc(C)cc3c(=O)n2C)cc1F] C₂₆H₂₄FN₃O₄ | 246 | | ES− 460.2 [M − H] | ¹H NMR (400 MHz, DMSO-d₆) δ 9.05 (s, 1H), 8.36 (d, J = 2.9 Hz, 1H), 7.98-7.96 (m, 2H), 7.78 (d, J = 7.8 Hz, 1H), 7.52 (s, 1H), 7.07-7.04 (m, 1H), 6.94 (d, J = 13.7 Hz, 1H), 6.47- 6.41 (m, 1H), 6.28- 6.19 (m, 1H), 5.22- 5.06 (m, 1H), 3.90 (d, J = 7.1 Hz, 3H), 3.27 (s, 3H), 2.37 (s, 3H), 1.51 (d, J = 6.6 Hz, 3H). |
| 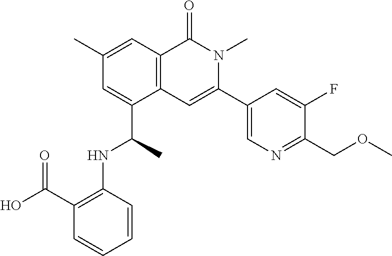 [SMILES: COCc1ncc(-c2cc3c([C@@H](C)Nc4ccccc4C(=O)O)cc(C)cc3c(=O)n2C)cc1F] C₂₇H₂₆FN₃O₄ | 247 | | ES+ 476.2 [M + H] | ¹H NMR (400 MHz, DMSO-d₆) δ 12.76 (s, 1H), 8.67 (d, J = 1.6 Hz, 1H), 8.57- 8.51 (m, 1H), 8.12- 8.08 (m, 1H), 8.01 (d, J = 1.8 Hz, 1H), 7.81-7.78 (m, 1H), 7.51 (d, J = 1.9 Hz, 1H), 7.18-7.09 (m, 1H), 7.02 (s, 1H), 6.50 (d, J = 7.5 Hz, 1H), 6.33 (d, J = 8.5 Hz, 1H), 5.24-5.15 (m, 1H), 4.63-4.62 (m, 2H), 3.37 (s, 6H), 2.38 (s, 3H), 1.50 (d, J = 6.5 Hz, 3H). |
| 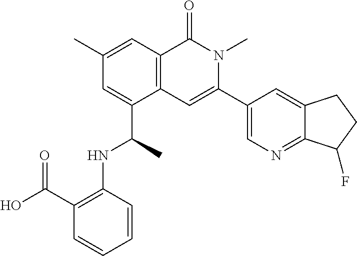 [SMILES: Cc1cc([C@@H](C)Nc2ccccc2C(=O)O)c2cc(-c3cnc4c(c3)CCC4F)n(C)c(=O)c2c1] C₂₈H₂₆FN₃O₃ | 248 | mixture of diastereomers | ES+ 471.9 [M + H] | ¹H NMR (400 MHz, DMSO-d₆) δ 8.73 (s, 2H), 8.06 (s, 1H), 8.00 (s, 1H), 7.81 (d, J = 7.9 Hz, 1H), 7.51 (s, 1H), 7.08 (d, J = 8.2 Hz, 1H), 6.95 (s, 1H), 6.52-6.45 (m, 1H), 6.29 (d, J = 8.5 Hz, 1H), 6.11-5.94 (m, 1H), 5.20-5.10 (m, 1H), 3.35 (s, 3H), 3.17 (s, 1H), 3.04-2.98 (m, 1H), 2.55 (s, 1H), 2.37 (s, 3H), 2.25-2.15 (m, 1H), 1.49 (d, J = 6.5 Hz, 3H). |
| 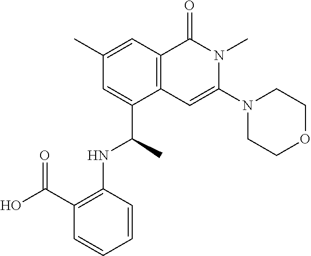 [SMILES: Cc1cc([C@@H](C)Nc2ccccc2C(=O)O)c2cc(N3CCOCC3)n(C)c(=O)c2c1] C₂₄H₂₇N₃O₄ | 249 | | ES+ 422.5 [M + H] | ¹H NMR (400 MHz, DMSO-d₆) δ 12.77 (s, 1H), 8.41 (s, 1H), 7.88 (d, J = 1.8 Hz, 1H), 7.81-7.79 (m, 1H), 7.43 (d, J = 1.9 Hz, 1H), 7.22-7.18 (m, 1H), 6.56-6.48 (m, 1H), 6.45-6.36 (m, 2H), 5.15-5.05 (m, 1H), 3.76-3.67 (m, 4H), 3.52 (s, 3H), 3.17-2.95 (m, 4H), 2.33 (s, 3H), 1.55 (d, J = 6.6 Hz, 3H). |
| 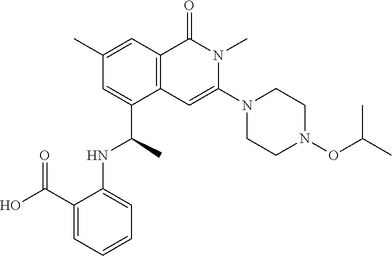 [SMILES: Cc1cc([C@@H](C)Nc2ccccc2C(=O)O)c2cc(N3CCN(OC(C)C)CC3)n(C)c(=O)c2c1] C₂₇H₃₄N₄O₄ | 250 | | ES− 477.0 [M − H] | ¹H NMR (400 MHz, Methanol-d₄) δ 7.96- 7.92 (m, 1H), 7.92- 7.85 (m, 1H), 7.54- 7.50 (m, 1H), 7.15- 7.13 (m, 1H), 6.55- 6.51 (m, 1H), 6.50 (s, 1H), 6.40-6.35 (m, 1H), 5.13-5.08 (m, 1H), 3.99-3.94 (m, 1H), 3.62 (s, 3H), 3.25-3.15 (m, 4H), 2.93-2.70 (m, 4H) 2.37 (s, 3H), 1.63 (d, J = 6.8 Hz, 3H), 1.20-1.15 (m, 6H). |
| 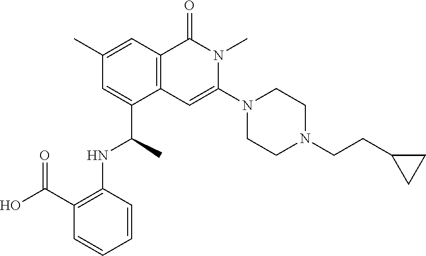 [SMILES: Cc1cc([C@@H](C)Nc2ccccc2C(=O)O)c2cc(N3CCN(CCC4CC4)CC3)n(C)c(=O)c2c1] C₂₈H₃₄N₄O₄ | 251 | | ES− 489.1 [M − H] | ¹H NMR (400 MHz, Methanol-d₄) δ 7.97 (s, 1H), 7.92-7.88 (m, 1H), 7.54-7.50 (m, 1H), 7.16-7.12 (m, 1H), 6.67-6.44 (m, 2H), 6.40-6.35 (m, 1H), 5.12-5.09 (m, 1H), 3.62 (s, 3H), 3.59-3.54 (m, 2H), 3.37-3.36 (m, 2H), 3.21-3.20 (m, 2H), 2.88-2.84 (m, 4H), 2.37 (s, 3H), 1.63 (d, J = 6.8 Hz, 3H), 1.14-0.93 (m, 1H), 0.76-0.40 (m, 2H), 0.35-0.28 (m, 2H). |
| 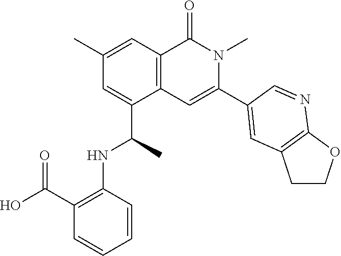 [SMILES: Cc1cc([C@@H](C)Nc2ccccc2C(=O)O)c2cc(-c3cnc4c(c3)CCO4)n(C)c(=O)c2c1] C₂₇H₂₅N₃O₄ | 252 | | ES− 454.3 [M − H] | ¹H NMR (400 MHz, DMSO-d₆) δ 8.74 (s, 1H), 8.13 (d, J = 6.5, 2.2 Hz, 1H), 7.98 (s, 1H), 7.87 (d, J = 2.1 Hz, 1H), 7.81 (d, J = 7.8 Hz, 1H), 7.50 (d, J = 1.9 Hz, 1H), 7.10 (d, J = 7.9 Hz, 1H), 6.85 (s, 1H), 6.48 (d, J = 7.5 Hz, 1H), 6.29 (d, J = 8.5 Hz, 1H), 5.17-5.10 (m, 1H), 4.75-4.55 (m, 2H), 3.36 (s, 3H), 3.32- 3.25 (m, 2H), 2.36 (s, 3H), 1.49 (d, J = 6.5 Hz, 3H). |
| 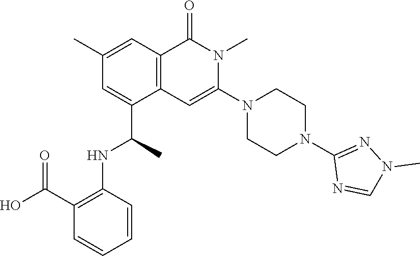 [SMILES: Cc1cc([C@@H](C)Nc2ccccc2C(=O)O)c2cc(N3CCN(c4ncn(C)n4)CC3)n(C)c(=O)c2c1] C₂₇H₃₁N₇O₃ | 253 | | ES+ 502.4 [M + H] | ¹H NMR (400 MHz, DMSO-d₆) δ 8.55 (s, 1H), 8.12 (s, 1H), 7.88 (s, 1H), 7.80 (d, J = 7.8 Hz, 1H), 7.43 (d, J = 1.9 Hz, 1H), 7.18-7.14 (m, 1H), 6.55-6.48 (m, 1H), 6.42-6.33 (m, 2H), 5.14-5.08 (m, 1H), 4.12-3.75 (m, 2H), 3.70 (s, 3H), 3.55 (s, 3H), 3.25-2.70 (m, 6H), 2.33 (s, 3H), 1.54 (d, J = 6.5 Hz, 3H). |
| 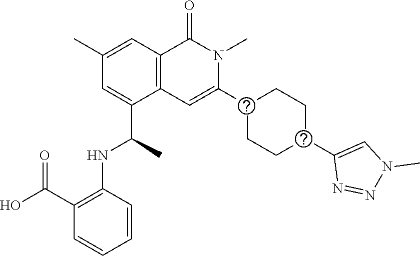 [SMILES: Cc1cc([C@@H](C)Nc2ccccc2C(=O)O)c2cc(C3CCC(c4cn(C)nn4)CC3)n(C)c(=O)c2c1] C₂₇H₃₁N₇O₃ | 254 | | ES+ 502.4 [M + H] | ¹H NMR (400 MHz, DMSO-d₆) δ 8.71 (s, 1H), 7.88 (s, 1H), 7.80 (d, J = 7.8 Hz, 1H), 7.52 (s, 1H), 7.44 (s, 1H), 7.14 (d, J = 8.9 Hz, 1H), 6.51- 6.47 (m, 1H), 6.42 (s, 1H), 6.35 (d, J = 8.4 Hz, 1H), 5.13- 5.05 (m, 1H), 3.96 (s, 3H), 3.55-3.54 (m, 2H), 3.45 (s, 3H), 3.12-2.89 (m, 6H), 2.33 (s, 3H), 1.54 (d, J = 6.5 Hz, 3H). |
| 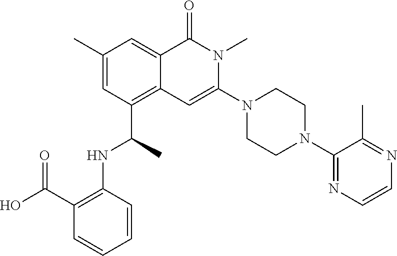 [SMILES: Cc1cc([C@@H](C)Nc2ccccc2C(=O)O)c2cc(N3CCN(c4nccnc4C)CC3)n(C)c(=O)c2c1] C₂₉H₃₂N₆O₃ | 255 | | ES+ 513.6 [M + H] | ¹H NMR (400 MHz, DMSO-d₆) δ 12.86 (s, 1H), 8.53 (s, 1H), 8.18-8.10 (m, 2H), 7.89 (s, 1H), 7.81 (d, J = 7.9 Hz, 1H), 7.43 (s, 1H), 7.19-7.16 (m, 1H), 6.52-6.49 (m, 1H), 6.45 (s, 1H), 6.38 (d, J = 8.5 Hz, 1H), 5.18-5.12 (m, 1H), 3.75-3.14 (m, 11H), 2.32 (s, 3H), 1.54 (d, J = 6.4 Hz, 3H). |
| 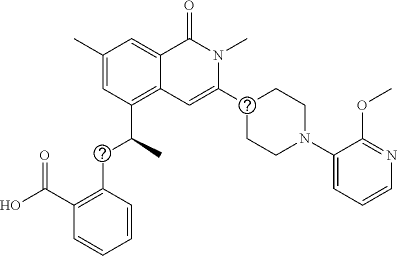 [SMILES: COc1ncccc1N1CCC(c2cc3c([C@@H](C)Cc4ccccc4C(=O)O)cc(C)cc3c(=O)n2C)CC1] C₃₀H₃₃N₅O₄ | 256 | | ES+ 528.6 [M + H] | ¹H NMR (400 MHz, DMSO-d₆) δ 12.76 (s, 1H), 8.43 (s, 1H), 7.89 (s, 1H), 7.84- 7.76 (m, 2H), 7.45- 7.40 (m, 1H), 7.27 (d, J = 7.6 Hz, 1H), 7.21-7.19 (m, 1H), 6.97-6.93 (m, 1H), 6.55-6.50 (m, 1H), 6.48 (s, 1H), 6.42 (d, J = 8.5 Hz, 1H), 5.25- 5.18 (m, 1H), 3.91 (s, 3H), 3.58-3.50 (m, 5H), 3.48-2.75 (m, 6H), 2.33 (s, 3H), 1.55 (d, J = 6.5 Hz, 3H). |
| 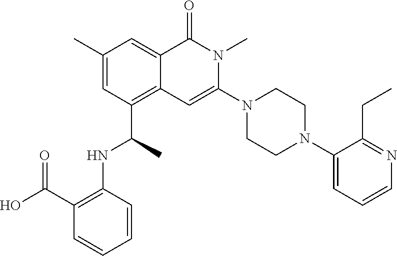 [SMILES: CCc1ncccc1N1CCN(c2cc3c([C@@H](C)Nc4ccccc4C(=O)O)cc(C)cc3c(=O)n2C)CC1] C₃₁H₃₅N₅O₃ | 257 | | ES+ 526.2 [M + H] | ¹H NMR (400 MHz, DMSO-d₆) δ 9.03 (s, 1H), 8.28-8.22 (m, 1H), 7.87 (s, 1H), 7.84-7.76 (m, 1H), 7.57-7.51 (m, 1H), 7.45 (d, J = 1.9 Hz, 1H), 7.25-7.17 (m, 1H), 7.12-7.03 (m, 1H), 6.50-6.41 (m, 2H), 6.31 (d, J = 8.4 Hz, 1H), 5.15 (d, J = 7.0 Hz, 1H), 3.55 (s, 3H), 3.23-2.94 (m, 8H), 2.92-2.81 (m, 2H), 2.32 (s, 3H), 1.53 (d, J = 6.5 Hz, 3H), 1.30-1.22 (m, 3H). |
| 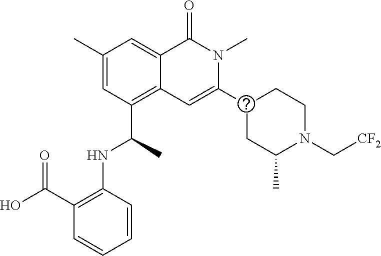 C₂₇H₃₁F₃N₄O₃ | 258 | | ES− 515.4 [M − H] | ¹H NMR (400 MHz, Methanol-d₄) δ 7.98 (d, J = 1.7 Hz, 1H), 7.94-7.88 (m, 1H), 7.56 (d, J = 1.9 Hz, 1H), 7.17-7.08 (m, 1H), 6.57-6.47 (m, 2H), 6.38 (d, J = 8.4 Hz, 1H), 5.15-5.05 (m, 1H), 3.65 (s, 3H), 3.21-2.79 (m, 6H), 2.51 (s, 3H), 2.38 (s, 3H), 1.65 (d, J = 6.6 Hz, 3H). |
| 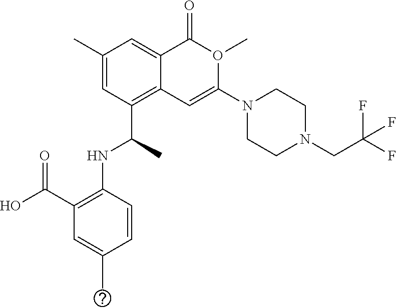 C₂₆H₂₈FAN₄O₃ | 259 | | ES+ 521.3 [M + H] | ¹H NMR (DMSO-d₆, 400 MHz, ppm): δ 13.09 (s, 1H), 8.19 (s, 1H), 7.92-7.84 (m, 1H), 7.58-7.51 (m, 1H), 7.40 (d, J = 1.9 Hz, 1H), 7.20- 7.10 (m, 1H), 6.47- 6.32 (m, 2H), 5.15- 5.05 (m, 1H), 3.49 (s, 3H), 3.30-3.28 (m, 2H), 3.19-2.75 (m, 8H), 2.32 (s, 3H), 1.52 (d, J = 6.5 Hz, 3H). |
| 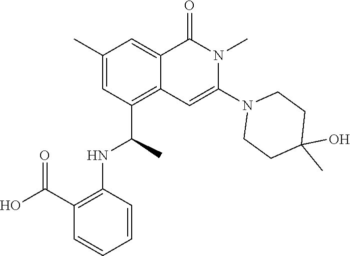 [SMILES: Cc1cc([C@@H](C)Nc2ccccc2C(=O)O)c2cc(N3CCC(C)(O)CC3)n(C)c(=O)c2c1] C₂₆H₃₁N₃O₄ | 260 | | ES+ 450.6 [M + H] | ¹H NMR (400 MHz, DMSO-d₆) δ 12.75 (s, 1H), 8.45 (s, 1H) 7.87-7.84 (m, 1H), 7.83-7.77 (m, 1H), 7.42-7.39 (m, 1H), 7.18-7.15 (m, 1H), 6.52-6.49 (m, 1H), 6.34 (d, J = 6.9 Hz, 2H), 5.15-5.05 (m, 1H), 3.48 (s, 3H), 3.10-2.82 (m, 4H), 2.31 (s, 3H), 1.75- 1.55 (m, 4H), 1.53 (d, J = 6.5 Hz, 3H), 1.21 (s, 3H). |
| 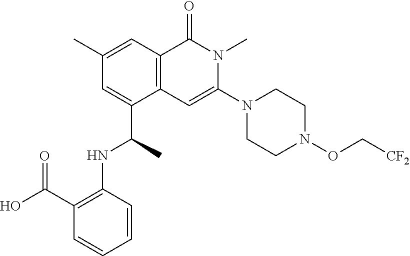 C₂₆H₂₉F₃N₄O₄ | 261 | | ES− 517.0 [M − H] | ¹H NMR (400 MHz, Methanol-d₄) δ 7.99- 7.95 (m, 1H), 7.92- 7.87 (m, 1H), 7.55- 7.51 (m, 1H), 7.16-7.13 (m, 1H), 6.62-6.46 (m, 2H), 6.40-6.35 (m, 1H), 5.20-5.05 (m, 1H), 4.22-4.19 (m, 2H), 3.62 (s, 3H), 3.44- 3.22 (s, 4H), 2.92- 2.85 (m, 4H), 2.37 (s, 3H), 1.65-1.61 (m, 3H). |
| 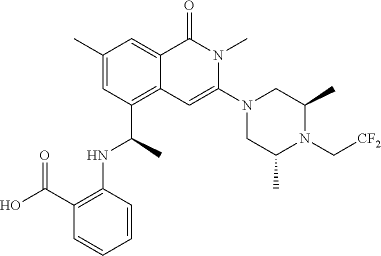 C₂₈H₃₃F₃N₄O₃ | 262 | | ES− 529.3 [M − H] | ¹H NMR (400 MHz, DMSO-d₆) δ 12.73 (s, 1H), 8.39 (d, J = 5.9 Hz, 1H), 7.87 (s, 1H), 7.83-7.77 (m, 1H), 7.41 (s, 1H), 7.23-7.14 (m, 1H), 6.56-6.48 (m, 1H), 6.37 (d, J = 7.6 Hz, 2H), 5.21-5.11 (m, 1H), 3.54 (s, 3H), 3.48-2.90 (m, 8H), 2.32 (s, 3H), 1.53 (d, J = 6.5 Hz, 3H), 1.40- 0.92 (m, 6H). |
| 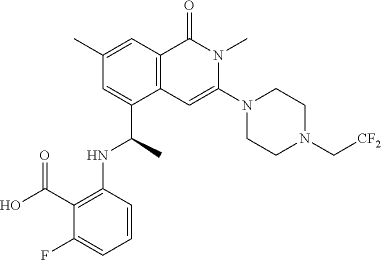 C₂₆H₂₈F₄N₄O₃ | 263 | | ES− 519.3 [M − H] | ¹H NMR (400 MHz, DMSO-d₆) δ 8.22 (s, 1H), 7.86 (s, 1H), 7.42 (d, J = 1.9 Hz, 1H), 7.05 (d, J = 7.7 Hz, 1H), 6.39 (s, 1H), 6.30-6.23 (m, 1H), 6.11 (d, J = 8.6 Hz, 1H), 5.15-5.05 (m, 1H), 3.49 (s, 3H), 3.29-3.20 (m, 2H), 3.15-2.60 (m, 8H), 2.32 (s, 3H), 1.50 (d, J = 6.5 Hz, 3H). |
| 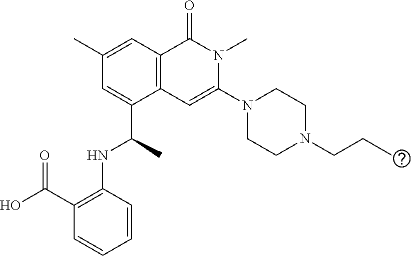 [SMILES: CCCN1CCN(c2cc3c([C@@H](C)Nc4ccccc4C(=O)O)cc(C)cc3c(=O)n2C)CC1] C₂₇H₃₁F₃N₄O₃ | 264 | | ES− 515.3 [M − H] | ¹H NMR (400 MHz, Methanol-d₄) δ 7.97 (s, 1H), 7.93-7.86 (m, 1H), 7.54 (d, J = 1.9 Hz, 1H), 7.13- 7.11 (m, 1H), 6.54 (s, 1H), 6.53-6.49 (m, 1H), 6.36 (d, J = 8.5 Hz, 1H), 5.11- 5.09 (m, 1H), 3.62 (s, 3H), 3.33-2.80 (m, 6H), 2.76-2.67 (m, 2H), 2.52-2.37 (m, 7H), 1.63 (d, J = 6.7 Hz, 3H). |
| 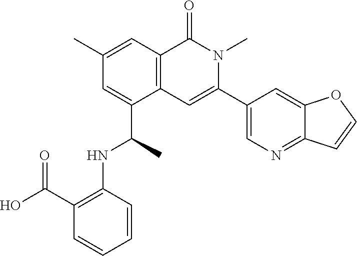 [SMILES: Cc1cc([C@@H](C)Nc2ccccc2C(=O)O)c2cc(-c3cnc4ccoc4c3)n(C)c(=O)c2c1] C₂₇H₂₃N₃O₄ | 265 | | ES− 452.2 [M − H] | ¹H NMR (400 MHz, DMSO-d₆) δ 12.73 (s, 1H), 8.77 (d, J = 1.8 Hz, 1H), 8.46 (d, J = 2.3 Hz, 1H), 8.39- 8.38 (m, 2H), 8.02 (s, 1H), 7.81-7.79 (m, 1H), 7.49 (d, J = 1.9 Hz, 1H), 7.28- 7.27 (m, 1H), 7.20- 7.17 (m, 1H), 7.00 (s, 1H), 6.54-6.50 (m, 1H), 6.35 (d, J = 8.5 Hz, 1H), 5.26- 5.23 (m, 1H), 3.39 (s, 3H), 2.39 (s, 3H), 1.51 (d, J = 6.6 Hz, 3H). |
| 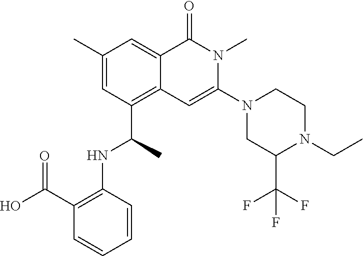 [SMILES: CCN1CCN(c2cc3c([C@@H](C)Nc4ccccc4C(=O)O)cc(C)cc3c(=O)n2C)CC1C(F)(F)F] C₂₇H₃₁F₃N₄O₃ | 266 | piperazine diastereomer 1 | ES+ 517.1 [M + H] | ¹H NMR (400 MHz, DMSO-d₆) δ 12.79 (s, 1H), 8.48 (s, 1H), 7.88 (s, 1H), 7.81- 7.78 (m, 1H), 7.42 (d, J = 1.9 Hz, 1H), 7.20-7.16 (m, 1H), 6.53-6.49 (m, 1H), 6.44 (s, 1H), 6.37 (d, J = 8.5 Hz, 1H), 5.19- 5.16 (m, 1H), 3.66- 3.65 (m, 1H), 3.49 (s, 3H), 3.32-3.19 (m, 1H), 3.14-3.05 (m, 3H), 2.82-2.81 (m, 4H), 2.33 (s, 3H), 1.53 (d, J = 6.6 Hz, 3H), 1.10-1.08 (m, 3H). |
| 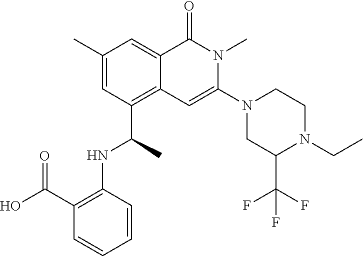 [SMILES: CCN1CCN(c2cc3c([C@@H](C)Nc4ccccc4C(=O)O)cc(C)cc3c(=O)n2C)CC1C(F)(F)F] C₂₇H₃₁F₃N₄O₃ | 267 | piperazine diastereomer 2 | ES+ 517.1 [M + H] | ¹H NMR (400 MHz, DMSO-d₆) δ 12.71 (s, 1H), 8.42 (s, 1H), 7.88 (s, 1H), 7.81- 7.79 (m, 1H), 7.42 (d, J = 1.9 Hz, 1H), 7.20-7.16 (m, 1H), 6.53-6.50 (m, 1H), 6.47 (s, 1H), 6.37 (d, J = 8.5 Hz, 1H), 5.21- 5.18 (m, 1H), 3.69- 3.64 (m, 1H), 3.49 (s, 3H), 3.32-3.22 (m, 2H), 3.09-3.06 (m, 2H), 2.82-2.80 (m, 4H), 2.33 (s, 3H), 1.54 (d, J = 6.6 Hz, 3H), 1.10-1.08 (m, 3H). |
| 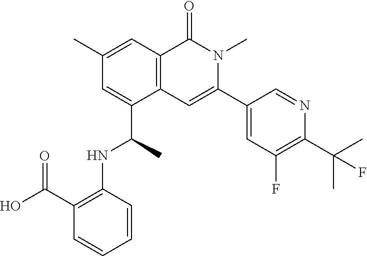 [SMILES: Cc1cc([C@@H](C)Nc2ccccc2C(=O)O)c2cc(-c3cnc(C(C)(C)F)c(F)c3)n(C)c(=O)c2c1] C₂₈H₂₇F₂N₃O₃ | 268 | | ES− 490.2 [M − H] | ¹H NMR (400 MHz, Methanol-d₄) δ 8.60- 8.54 (m, 1H), 8.10 (s, 1H), 7.92-7.86 (m, 2H), 7.65-7.59 (m, 1H), 7.13-7.08 (m, 1H), 7.04 (s, 1H), 6.56-6.46 (m, 1H), 6.35-6.30 (m, 1H), 5.20-5.12 (m, 1H), 3.48 (s, 3H), 2.42 (s, 3H), 1.87- 1.83 (m, 3H), 1.82- 1.79 (m, 3H), 1.62- 1.58 (m, 3H). |
| 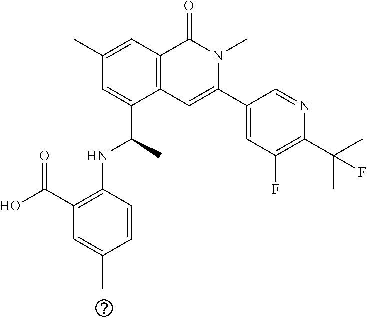 [SMILES: Cc1ccc(N[C@H](C)c2cc(C)cc3c(=O)n(C)c(-c4cnc(C(C)(C)F)c(F)c4)cc23)c(C(=O)O)c1] C₂₈H₂₆F₃N₃O₃ | 269 | | ES− 508.1 [M − H] | ¹H NMR (300 MHz, Methanol-d₄) δ 8.58 (s, 1H), 8.12 (s, 1H), 7.93-7.86 (m, 1H), 7.68-7.46 (m, 2H), 7.05 (s, 1H), 7.02- 6.84 (m, 1H), 6.35- 6.29 (m, 1H), 5.22- 5.15 (m, 1H), 3.50 (s, 3H), 2.45 (s, 3H), 1.95 (s, 3H), 1.88 (s, 3H), 1.61 (d, J = 8.8 Hz, 3H). |
| 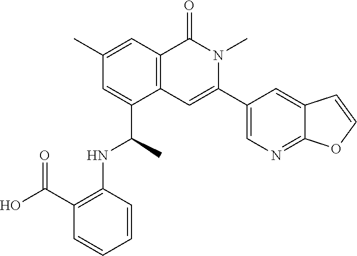 [SMILES: Cc1cc([C@@H](C)Nc2ccccc2C(=O)O)c2cc(-c3cnc4occc4c3)n(C)c(=O)c2c1] C₂₇H₂₃N₃O₄ | 270 | | ES+ 454.2 [M + H] | ¹H NMR (400 MHz, Methanol-d₄) δ 8.48 (d, J = 2.1 Hz, 1H), 8.31 (d, J = 2.2 Hz, 1H), 8.12 (d, J = 1.6 Hz, 1H), 8.04 (d, J = 2.5 Hz, 1H), 7.93- 7.87 (m, 1H), 7.66 (d, J = 2.0 Hz, 1H), 7.13-7.03 (m, 3H), 6.56-6.47 (m, 1H), 6.32 (d, J = 8.0 Hz, 1H), 5.21-5.11 (m, 1H), 3.48 (s, 3H), 2.44 (s, 3H), 1.62 (d, J = 6.7 Hz, 3H). |
| 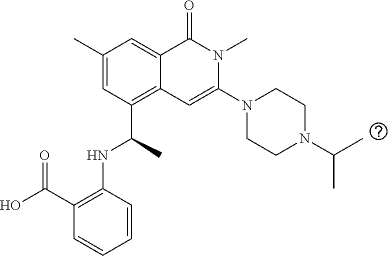 C₂₇H₃₁F₃N₄O₃ | 271 | mixture of piperazine diastereomers | ES+ 517.1 [M + H] | ¹H NMR (400 MHz, DMSO-d₆) δ 12.74 (s, 1H), 8.45-8.30 (m, 1H), 7.94-7.84 (m, 1H), 7.84-7.76 (m, 1H), 7.45-7.37 (m, 1H), 7.25-7.15 (m, 1H), 6.56-6.48 (m, 1H), 6.44-6.34 (m, 2H), 5.24-5.12 (m, 1H), 3.64-3.52 (m, 1H), 3.50 (s, 3H), 3.25-2.60 (m, 8H), 2.32 (s, 3H), 1.60-1.47 (m, 3H), 1.29-1.18 (m, 3H). |
| 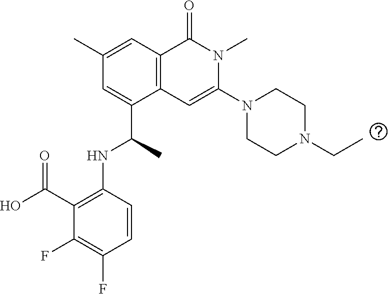 C₂₆H₂₇F₅N₄O₃ | 272 | | ES+ 539.4 [M + H] | ¹H NMR (400 MHz, Methanol-d₄) δ 7.97 (d, J = 1.7 Hz, 1H), 7.52 (d, J = 1.8 Hz, 1H), 7.02-6.95 (m, 1H), 6.49 (s, 1H), 6.08-6.00 (m, 1H), 5.08-4.99 (m, 1H), 3.62 (s, 3H), 3.20- 2.65 (m, 10H), 2.37 (s, 3H), 1.60 (d, J = 6.7 Hz, 3H). |
| 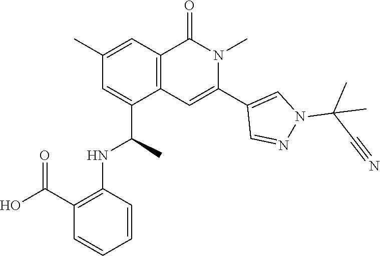 [SMILES: Cc1cc([C@@H](C)Nc2ccccc2C(=O)O)c2cc(-c3cnn(C(C)(C)C#N)c3)n(C)c(=O)c2c1] C₂₇H₂₇N₅O₃ | 273 | | ES+ 470.5 [M + H] | ¹H NMR (400 MHz, DMSO-d₆) δ 12.77 (s, 1H), 8.45 (s, 1H), 8.40 (d, J = 5.9 Hz, 1H), 8.04 (s, 1H), 7.97 (s, 1H), 7.82- 7.80 (m, 1H), 7.46 (d, J = 1.9 Hz, 1H), 7.20-7.17 (m, 1H), 6.97 (s, 1H), 6.53- 6.50 (m, 1H), 6.34 (d, J = 8.5 Hz, 1H), 5.26-5.24 (m, 1H), 3.55 (s, 3H), 2.36 (s, 3H), 2.05 (s, 6H), 1.53 (d, J = 6.5 Hz, 3H). |
| 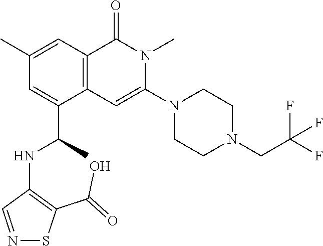 [SMILES: Cc1cc([C@@H](C)Nc2cnsc2C(=O)O)c2cc(N3CCN(CC(F)(F)F)CC3)n(C)c(=O)c2c1] C₂₃H₂₆F₃N₅O₃S | 274 | | ES+ 510.2 [M + H] | ¹H NMR (400 MHz, Methanol-d₄) δ 8.01 (d, J = 1.7 Hz, 1H), 7.86 (s, 1H), 7.58- 7.57 (m, 1H), 6.51 (s, 1H), 5.29-5.23 (m, 1H), 3.64 (s, 3H), 3.23-2.82 (m, 10H), 2.41 (s, 3H), 1.62 (d, J = 6.7 Hz, 3H). |
| 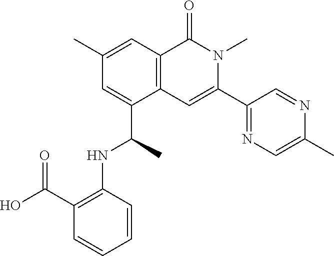 [SMILES: Cc1cc([C@@H](C)Nc2ccccc2C(=O)O)c2cc(-c3cnc(C)cn3)n(C)c(=O)c2c1] C₂₅H₂₄N₄O₃ | 275 | | ES+ 429.5 [M + H] | ¹H NMR (400 MHz, DMSO-d₆) δ 12.85 (s, 1H), 8.92 (d, J = 1.5 Hz, 1H), 8.71 (d, J = 1.4 Hz, 1H), 8.56 (s, 1H), 8.02 (d, J = 1.9 Hz, 1H), 7.82- 7.80 (m, 1H), 7.53 (d, J = 1.9 Hz, 1H), 7.15-7.12 (m, 2H), 6.53-6.50 (m, 1H), 6.34 (d, J = 8.5 Hz, 1H), 5.27-5.15 (m, 1H), 3.43 (s, 3H), 2.62 (s, 3H), 2.39 (s, 3H), 1.52 (d, J = 6.5 Hz, 3H). |
| 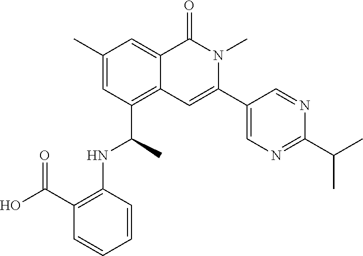 [SMILES: Cc1cc([C@@H](C)Nc2ccccc2C(=O)O)c2cc(-c3cnc(C(C)C)nc3)n(C)c(=O)c2c1] C₂₇H₂₈N₄O₃ | 276 | | ES+ 457.0 [M + H] | ¹H NMR (400 MHz, DMSO-d₆) δ 12.82 (s, 1H), 9.03 (s, 2H), 8.44 (s, 1H), 8.01 (d, J = 1.9 Hz, 1H), 7.83- 7.79 (m, 1H), 7.50 (d, J = 1.9 Hz, 1H), 7.19-7.13 (m, 1H), 7.07 (s, 1H), 6.56- 6.48 (m, 1H), 6.35 (d, J = 8.5 Hz, 1H), 5.28-5.15 (m, 1H), 3.39 (s, 3H), 3.30- 3.20 (m, 1H), 2.38 (s, 3H), 1.51 (d, J = 6.5 Hz, 3H), 1.36 (d, J = 6.9 Hz, 6H). |
| 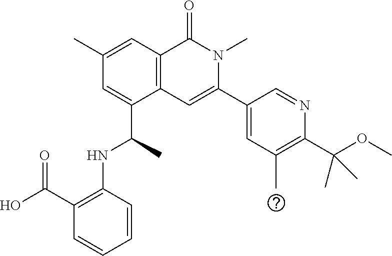 [SMILES: COC(C)(C)c1ncc(-c2cc3c([C@@H](C)Nc4ccccc4C(=O)O)cc(C)cc3c(=O)n2C)cc1C] C₂₉H₃₀FN₃O₄ | 277 | | ES− 502.2 [M − H] | ¹H NMR (400 MHz, Methanol-d₄) δ 8.60- 8.55 (m, 1H), 8.16- 8.04 (m, 1H), 7.92- 7.86 (m, 1H), 7.83- 7.80 (m, 1H), 7.63- 7.59 (m, 1H), 7.15- 7.08 (m, 1H), 7.03 (s, 1H), 6.53-6.48 (m, 1H), 6.35-6.30 (m, 1H), 5.20-5.15 (m, 1H), 3.48 (s, 3H), 3.18 (s, 3H), 2.42 (s, 3H), 1.70- 1.65 (m, 6H), 1.62- 1.58 (m, 3H). |
| 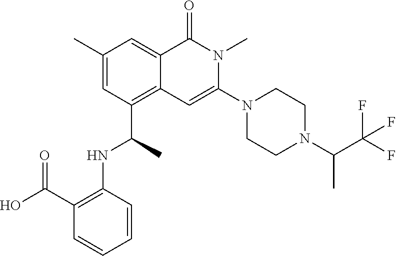 [SMILES: Cc1cc([C@@H](C)Nc2ccccc2C(=O)O)c2cc(N3CCN(C(C)C(F)(F)F)CC3)n(C)c(=O)c2c1] C₂₇H₃₁F₃N₄O₃ | 278 | piperazine diastereomer 1 | ES− 515.3 [M − H] | ¹H NMR (400 MHz, Methanol-d₄) δ 8.01- 7.96 (m, 1H), 7.94- 7.87 (m, 1H), 7.56- 7.52 (m, 1H), 7.18- 7.12 (m, 1H), 6.56- 6.48 (m, 2H), 6.41- 6.35 (m, 1H), 5.15- 5.08 (m, 1H), 3.64 (s, 3H), 3.46-3.38 (m, 1H) , 3.22-2.76 (m, 8H), 2.38 (s, 3H), 1.65 (d, J = 6.7 Hz, 3H), 1.35-1.28 (m, 3H). |
| 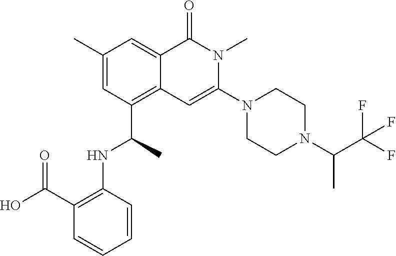 [SMILES: Cc1cc([C@@H](C)Nc2ccccc2C(=O)O)c2cc(N3CCN(C(C)C(F)(F)F)CC3)n(C)c(=O)c2c1] C₂₇H₃₁F₃N₄O₃ | 279 | piperazine diastereomer 2 | ES+ 517.1 [M + H] | ¹H NMR (400 MHz, Methanol-d₄) δ 8.01- 7.96 (m, 1H), 7.95- 7.85 (m, 1H), 7.56- 7.52 (m, 1H), 7.20- 7.10 (m, 1H), 6.58- 6.48 (m, 2H), 6.42- 6.34 (m, 1H), 5.16- 5.07 (m, 1H), 3.64 (s, 3H), 3.51-3.37 (m, 1H), 3.24-2.56 (m, 8H), 2.39 (s, 3H), 1.65 (d, J = 6.7 Hz, 3H), 1.32 (d, J = 7.1 Hz, 3H). |
| 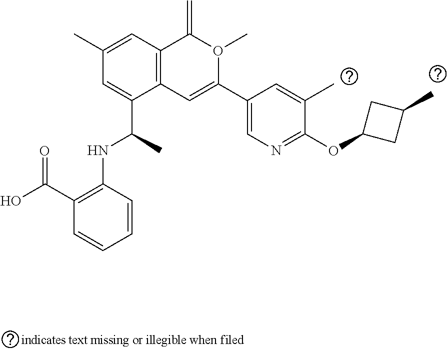 C₂₉H₂₇F₂N₃O₄ | 280 | | ES+ 520.2 [M + H] | ¹H NMR (400 MHz, Methanol-d₄) δ 8.60- 8.55 (m, 1H), 8.16- 8.04 (m, 1H), 7.92- 7.86 (m, 1H), 7.83- 7.80 (m, 1H), 7.63- 7.59 (m, 1H), 7.15- 7.08 (m, 1H), 7.03 (s, 1H), 6.53-6.48 (m, 1H), 6.35-6.30 (m, 1H), 5.20-5.15 (m, 3H), 3.48 (s, 3H), 3.15-3.10 (m, 2H), 2.45-2.37 (m, 5H), 1.61 (d, J = 6.4 Hz, 3H). |
| 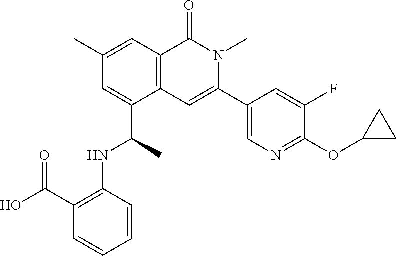 [SMILES: Cc1cc([C@@H](C)Nc2ccccc2C(=O)O)c2cc(-c3cnc(OC4CC4)c(F)c3)n(C)c(=O)c2c1] C₂₈H₂₆FN₃O₄ | 281 | | ES− 486.0 [M − H] | ¹H NMR (400 MHz, DMSO-d₆) δ 12.72 (s, 1H), 8.42-8.37 (m, 1H), 8.32-8.26 (m, 1H), 8.12-8.06 (m, 1H), 8.02-7.99 (m, 1H), 7.82-7.78 (m, 1H), 7.50-7.46 (m, 1H), 7.20-7.16 (m, 1H), 6.98 (s, 1H), 6.55-6.50 (m, 1H), 6.36-6.32 (m, 1H), 5.27-5.23 (m, 1H), 4.45-4.42 (m, 1H), 3.38 (s, 3H), 2.37 (s, 3H), 1.52- 1.48 (m, 3H), 0.92- 0.73 (m, 4H). |
| 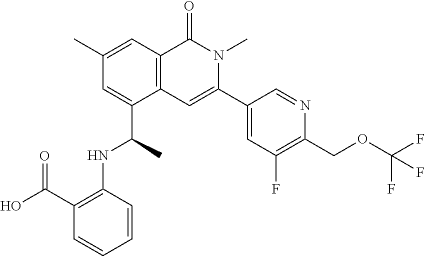 [SMILES: Cc1cc([C@@H](C)Nc2ccccc2C(=O)O)c2cc(-c3cnc(COC(F)(F)F)c(F)c3)n(C)c(=O)c2c1] C₂₇H₂₃F₄N₃O₄ | 282 | | ES+ 530.2 [M + H] | ¹H NMR (400 MHz, Methanol-d₄) δ 8.68- 8.64 (m, 1H), 8.10 (s, 1H), 8.02-7.98 (m, 1H), 7.90-7.87 (m, 1H), 7.65-7.62 (m, 1H), 7.14-7.04 (m, 2H), 6.55-6.49 (m, 1H), 6.35-6.30 (m, 1H), 5.33-5.29 (m, 2H), 5.20-5.15 (m, 1H), 3.47 (s, 3H), 2.43 (s, 3H), 1.61 (d, J = 6.6 Hz, 3H). |
| 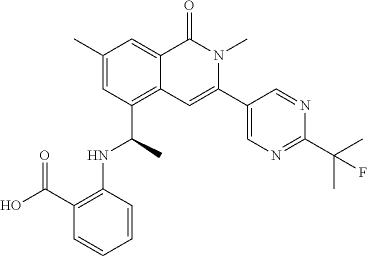 [SMILES: Cc1cc([C@@H](C)Nc2ccccc2C(=O)O)c2cc(-c3cnc(C(C)(C)F)nc3)n(C)c(=O)c2c1] C₂₇H₂₇FN₄O₃ | 283 | | ES− 473.2 [M − H] | ¹H NMR (300 MHz, Methanol-d₄) δ 9.06 (s, 2H), 8.13 (s, 1H), 7.95-7.90 (m, 1H), 7.65 (s, 1H), 7.12 (s, 2H), 6.56-6.52 (m, 1H), 6.40-6.33 (m, 1H), 5.25-5.18 (m, 1H), 3.52 (s, 3H), 2.45 (s, 3H), 1.89 (s, 3H), 1.82 (s, 3H), 1.65-1.60 (m, 3H). |
| 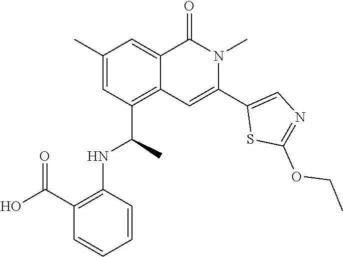 [SMILES: CCOc1ncc(-c2cc3c([C@@H](C)Nc4ccccc4C(=O)O)cc(C)cc3c(=O)n2C)s1] C₂₅H₂₅N₃O₄S | 284 | | ES− 462.2 [M − H] | ¹H NMR (300 MHz, DMSO-d₆) δ 12.76 (s, 1H), 8.40-8.36 (m, 1H), 7.98 (s, 1H), 7.83-7.79 (m, 1H), 7.52-7.49 (m, 2H), 7.25-7.15 (m, 1H), 7.09 (s, 1H), 6.62-6.55 (m, 1H), 6.40-6.33 (m, 1H), 5.32-5.15 (m, 1H), 4.52-4.50 (m, 2H), 3.49 (s, 3H), 2.38 (s, 3H), 1.54-1.50 (m, 3H), 1.45-1.39 (m, 3H). |
| 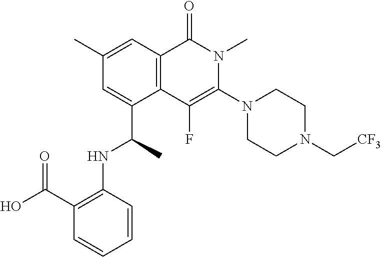 [SMILES: Cc1cc([C@@H](C)Nc2ccccc2C(=O)O)c2c(F)c(N3CCN(CC(F)(F)F)CC3)n(C)c(=O)c2c1] C₂₆H₂₈F₄N₄O₃ | 285 | | ES+ 521.8 [M + H] | ¹H NMR (400 MHz, Methanol-d₄) δ 8.09- 8.03 (m, 1H), 7.92- 7.89 (m, 1H), 7.69- 7.68 (m, 1H), 7.13- 7.09 (m, 1H), 6.54- 6.50 (m, 1H), 6.30- 6.27 (m, 1H), 5.40- 5.39 (m, 1H), 3.65 (s, 3H), 3.54-3.43 (m, 2H), 3.24-3.06 (m, 4H), 3.08-2.99 (m, 2H), 2.79-2.73 (m, 2H), 2.36 (s, 3H), 1.63-1.57 (m, 3H). |
| 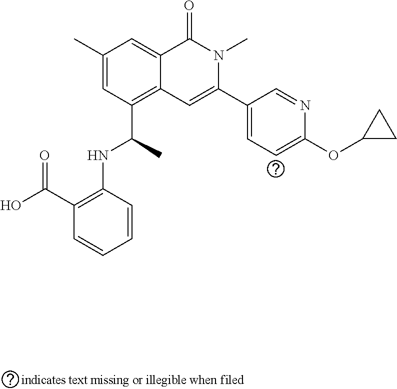 [SMILES: Cc1cc([C@@H](C)Nc2ccccc2C(=O)O)c2cc(-c3ccc(OC4CC4)nc3)n(C)c(=O)c2c1] C₂₇H₂₆N₄O₄ | 286 | | ES+ 471.5 [M + H] | ¹H NMR (400 MHz, Methanol-d₄) δ 8.82 (s, 2H), 8.09 (s, 1H), 7.91-7.88 (m, 1H), 7.72-7.69 (m, 1H), 7.08 (s, 1H), 7.05- 7.01 (m, 1H), 6.55- 6.42 (m, 1H), 6.27 (d, J = 8.4 Hz, 1H), 5.18-5.12 (m, 1H), 4.52-4.45 (m, 1H), 3.52 (s, 3H), 2.43 (s, 3H), 1.62 (d, J = 6.6 Hz, 3H), 0.94-0.82 (m, 4H). |
| 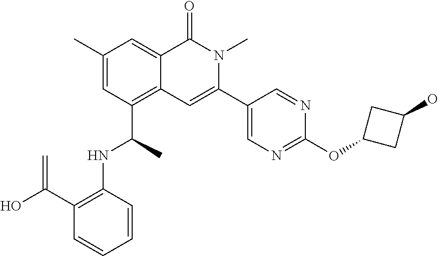 C₂₈H₂₇FN₄O₄ | 287 | | ES+ 503.6 [M + H] | ¹H NMR (400 MHz, DMSO-d₆) δ 12.78 (s, 1H), 8.90 (s, 2H), 8.45-8.38 (m, 1H), 8.00 (s, 1H), 7.82- 7.80 (m, 1H), 7.49 (d, J = 1.9 Hz, 1H), 7.21-7.12 (m, 1H), 7.02 (s, 1H), 6.53- 6.50 (m, 1H), 6.34 (d, J = 8.5 Hz, 1H), 5.43-5.46 (m, 1H), 5.36-5.22 (m, 1H), 3.39 (s, 3H), 3.37- 3.35 (m, 1H), 2.81- 2.52 (m, 4H), 2.38 (s, 3H), 1.51 (d, J = 6.5 Hz, 3H). |
| 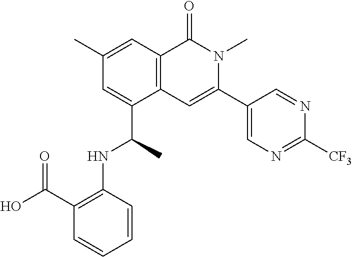 [SMILES: Cc1cc([C@@H](C)Nc2ccccc2C(=O)O)c2cc(-c3cnc(C(F)(F)F)nc3)n(C)c(=O)c2c1] C₂₅H₂₁F₃N₄O₃ | 288 | | ES− 481.1 [M − H] | ¹H NMR (400 MHz, DMSO-d₆) δ 12.76 (s, 1H), 9.39 (s, 2H), 8.45-8.40 (m, 1H), 8.06-8.00 (m, 1H), 7.83-7.80 (m, 1H), 7.54-7.51 (m, 1H), 7.27-7.10 (m, 2H), 6.57-6.47 (m, 1H), 6.36-6.32 (m, 1H), 5.28-5.24 (m, 1H), 3.44 (s, 3H), 2.39 (s, 3H), 1.55-1.50 (m, 3H). |
| 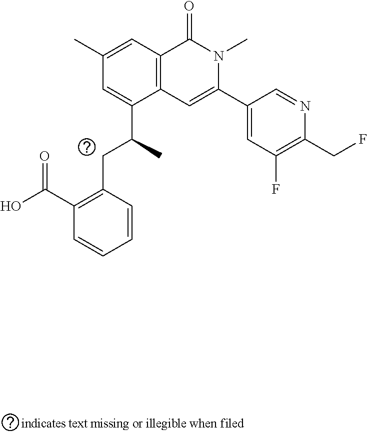 [SMILES: Cc1cc([C@@H](C)Cc2ccccc2C(=O)O)c2cc(-c3cnc(CF)c(F)c3)n(C)c(=O)c2c1] C₂₆H₂₃F₂N₃O₃ | 289 | | ES+ 464.2 [M + H] | ¹H NMR (400 MHz, Methanol-d₄) δ 8.64 (s, 1H), 8.10 (s, 1H), 7.99-7.95 (m, 1H), 7.93-7.86 (m, 1H), 7.66-7.60 (m, 1H), 7.15-7.09 (m, 1H), 7.06 (s, 1H), 6.54- 6.48 (m, 1H), 6.35- 6.31 (m, 1H), 5.60- 5.52 (m, 2H), 5.21- 5.15 (m, 1H), 3.47 (s, 3H), 2.43 (s, 3H), 1.61 (d, J = 6.7 Hz, 3H). |
| 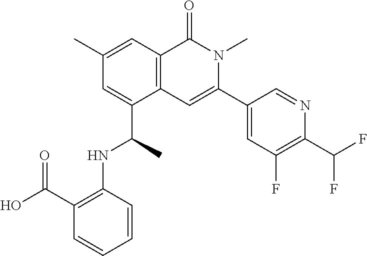 [SMILES: Cc1cc([C@@H](C)Nc2ccccc2C(=O)O)c2cc(-c3cnc(C(F)F)c(F)c3)n(C)c(=O)c2c1] C₂₆H₂₂F₃N₃O₃ | 290 | | ES+ 482.2 [M + H] | ¹H NMR (400 MHz, DMSO-d₆) δ 12.76 (s, 1H), 8.85-8.80 (m, 1H), 8.46-8.32 (m, 2H), 8.05-8.00 (m, 1H), 7.83-7.79 (m, 1H), 7.53-7.49 (m, 1H), 7.42-7.12 (m, 2H), 7.10 (s, 1H), 6.55-6.51 (m, 1H), 6.38-6.29 (m, 1H), 5.29-5.24 (m, 1H), 3.39 (s, 3H), 2.38 (s, 3H), 1.55- 1.50 (m, 3H). |
| 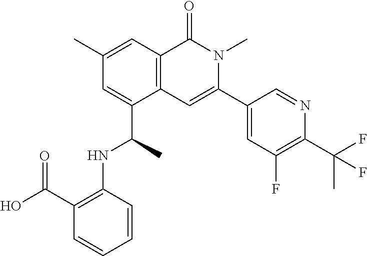 [SMILES: Cc1cc([C@@H](C)Nc2ccccc2C(=O)O)c2cc(-c3cnc(C(C)(F)F)c(F)c3)n(C)c(=O)c2c1] C₂₇H₂₄F₃N₃O₃ | 291 | | ES− 494.0 [M − H] | ¹H NMR (400 MHz, DMSO-d₆) δ 12.76 (s, 1H), 8.78 (s, 1H), 8.42-8.38 (m, 1H), 8.35-8.31 (m, 1H), 8.02 (s, 1H), 7.84- 7.78 (m, 1H), 7.52- 7.48 (m, 1H), 7.21- 7.12 (m, 1H), 7.09 (s, 1H), 6.55-6.50 (m, 1H), 6.36-6.32 (m, 1H), 5.30-5.25 (m, 1H), 3.40 (s, 3H), 2.38 (s, 3H), 2.15-2.11 (m, 3H), 1.53-1.48 (m, 3H). |
| 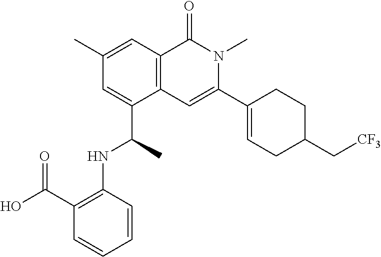 [SMILES: Cc1cc([C@@H](C)Nc2ccccc2C(=O)O)c2cc(C3=CCC(CC(F)(F)F)CC3)n(C)c(=O)c2c1] C₂₇H₂₈F₃N₃O₃ | 292 | | ES− 498.3 [M − H] | ¹H NMR (400 MHz, DMSO-d₆) δ 12.75 (s, 1H), 8.39 (d, J = 5.5 Hz, 1H), 7.93 (s, 1H), 7.83-7.78 (m, 1H), 7.44 (d, J = 1.9 Hz, 1H), 7.19-7.14 (m, 1H), 6.76 (s, 1H), 6.54-6.49 (m, 1H), 6.32 (d, J = 8.5 Hz, 1H), 5.98-5.92 (m, 1H), 5.23-5.15 (m, 1H), 3.41-3.31 (m, 7H), 2.93-2.90 (m, 2H), 2.40 (s, 2H), 2.34 (s, 3H), 1.51 (d, J = 6.5 Hz, 3H). |
| 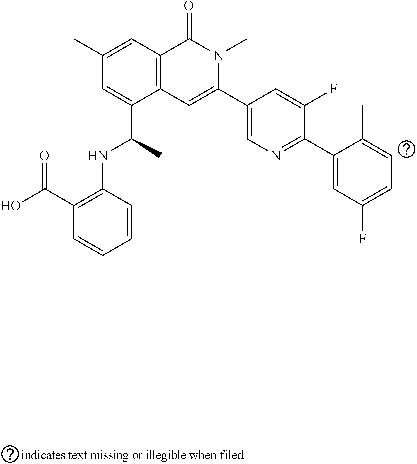 [SMILES: Cc1cc([C@@H](C)Nc2ccccc2C(=O)O)c2cc(-c3cnc(-c4cc(F)ccc4C)c(F)c3)n(C)c(=O)c2c1] C₃₁H₂₆F₂N₄O₃ | 293 | | ES+ 541.1 [M + H] | ¹H NMR (400 MHz, DMSO-d₆) δ 12.77 (s, 1H), 8.89-8.83 (m, 1H), 8.64 (d, J = 2.9 Hz, 1H), 8.53 (s, 1H), 8.34-8.30 (m, 1H), 8.03 (s, 1H), 7.87-7.85 (m, 1H), 7.83-7.75 (m, 1H), 7.52 (d, J = 1.9 Hz, 1H), 7.22-7.13 (m, 1H), 7.10 (s, 1H), 6.55-6.46 (m, 1H), 6.35 (d, J = 8.5 Hz, 1H), 5.29-5.20 (m, 1H), 3.45 (s, 3H), 2.46 (s, 3H), 2.39 (s, 3H), 1.53 (d, J = 6.5 Hz, 3H). |
| 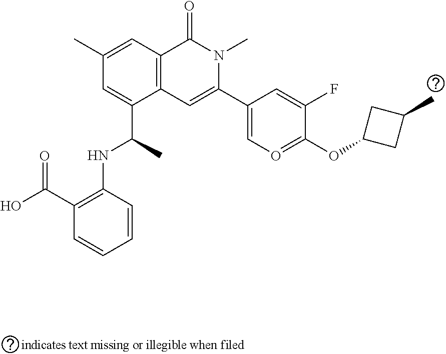 C₂₉H₂₇F₂N₃O₄ | 294 | | ES− 518.2 [M − H] | ¹H NMR (400 MHz, Methanol-d₄) δ 8.12- 8.07 (m, 2H), 7.93- 7.89 (m, 1H), 7.78- 7.75 (m, 1H), 7.62 (d, J = 2.0 Hz, 1H), 7.10-7.05 (m, 1H), 6.99 (s, 1H), 6.53- 6.49 (m, 1H), 6.30 (d, J = 8.4 Hz, 1H), 5.62-5.57 (m, 1H), 5.44-5.39 (m, 1H), 5.17-5.14 (m, 1H), 3.48 (s, 3H), 2.86- 2.55 (m, 4H), 2.42 (s, 3H), 1.61 (d, J = 6.6 Hz, 3H). |
| 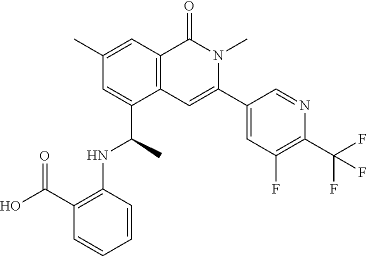 [SMILES: Cc1cc([C@@H](C)Nc2ccccc2C(=O)O)c2cc(-c3cnc(C(F)(F)F)c(F)c3)n(C)c(=O)c2c1] C₂₆H₂₁F₄N₃O₃ | 295 | | ES+ 501.1 [M + H] | ¹H NMR (400 MHz, Methanol-d₄) δ 8.74 (s, 1H), 8.17-8.15 (m, 1H), 8.10 (s, 1H), 7.89-7.87 (m, 1H), 7.63-7.62 (m, 1H), 7.12-7.08 (m, 2H), 6.53-6.51 (m, 1H), 6.33-6.31 (m, 1H), 5.19-5.15 (m, 1H), 3.48 (s, 3H), 2.43 (s, 3H), 1.61- 1.60 (d, J = 6.7 Hz, 3H). |
| 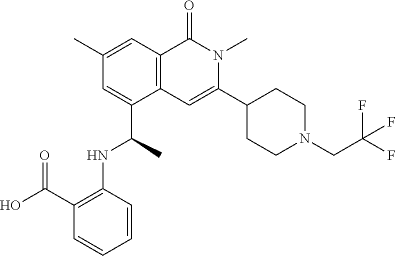 [SMILES: Cc1cc([C@@H](C)Nc2ccccc2C(=O)O)c2cc(C3CCN(CC(F)(F)F)CC3)n(C)c(=O)c2c1] C₂₇H₃₀F₃N₃O₃ | 296 | | ES− 500.3 [M − H] | ¹H NMR (DMSO-d₆, 400 MHz): δ 12.69 (s, 1H), 8.36 (d, J = 6.0 Hz, 1H), 7.92 (s, 1H), 7.85-7.77 (m, 1H), 7.44 (d, J = 1.9 Hz, 1H), 7.24-7.15 (m, 1H), 6.71 (s, 1H), 6.57-6.48 (m, 1H), 6.39 (d, J = 8.5 Hz, 1H), 5.31-5.22 (m, 1H), 3.58 (s, 3H), 3.25-3.20 (m, 2H), 3.06-3.01 (m, 2H), 2.83-2.77 (m, 1H), 2.67-2.56 (m, 2H), 2.34 (s, 3H), 1.92-1.84 (m, 2H), 1.83-1.63 (m, 2H), 1.53 (d, J = 6.5 Hz, 3H). |
| 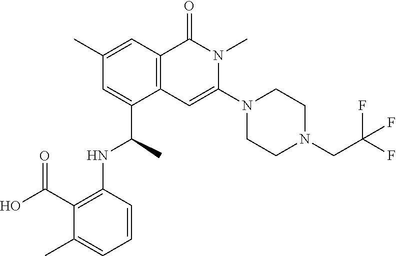 [SMILES: Cc1cc([C@@H](C)Nc2cccc(C)c2C(=O)O)c2cc(N3CCN(CC(F)(F)F)CC3)n(C)c(=O)c2c1] C₂₇H₃₁F₃N₄O₃ | 297 | | ES− 515.3 [M − H] | ¹H NMR (400 MHz, DMSO-d₆) δ 13.09 (s, 1H), 7.87 (s, 1H), 7.42 (d, J = 2.0 Hz, 1H), 7.24 (s, 1H), 7.01-6.95 (m, 1H), 6.40 (d, J = 7.6 Hz, 2H), 6.21 (d, J = 8.4 Hz, 1H), 5.12-5.02 (m, 1H), 3.49 (s, 3H), 3.45-3.22 (m, 2H), 3.21-2.58 (m, 8H), 2.40-2.30 (m, 6H), 1.50 (d, J = 6.5 Hz, 3H). |
| 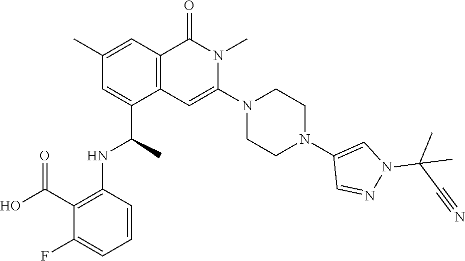 [SMILES: Cc1cc([C@@H](C)Nc2cccc(F)c2C(=O)O)c2cc(N3CCN(c4cnn(C(C)(C)C#N)c4)CC3)n(C)c(=O)c2c1] C₃₁H₃₅N₇O₃ | 298 | | ES− 552.4 [M − H] | ¹H NMR (400 MHz, DMSO-d₆) δ 12.71 (s, 1H), 8.39 (d, J = 5.5 Hz, 1H), 7.89 (s, 1H), 7.84-7.77 (m, 2H), 7.44 (d, J = 1.9 Hz, 1H), 7.26-7.16 (m, 1H), 6.56-6.49 (m, 1H), 6.42 (d, J = 7.9 Hz, 2H), 5.98 (d, J = 2.6 Hz, 1H), 5.21- 5.11 (m, 1H), 3.90- 3.48 (m, 5H), 3.40- 2.72 (m, 6H), 2.34 (s, 3H), 1.91 (s, 6H), 1.56 (d, J = 6.6 Hz, 3H). |
| 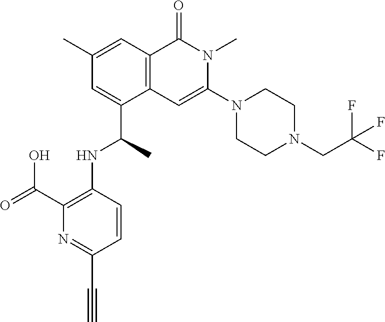 [SMILES: C#Cc1ccc(N[C@H](C)c2cc(C)cc3c(=O)n(C)c(N4CCN(CC(F)(F)F)CC4)cc23)c(C(=O)O)n1] C₂₇H₂₈F₃N₅O₃ | 299 | | ES+ 528.3 [M + H] | ¹H NMR (400 MHz, CDCl₃) δ 8.51-8.38 (m, 1H), 8.12 (s, 1H), 7.39 (s, 1H), 7.31-7.28 (m, 1H), 6.59 (d, J = 8.8 Hz, 1H), 6.23-6.17 (m, 1H), 4.98-4.87 (m, 1H), 3.67-3.60 (m, 3H), 3.17-2.78 (m, 11H), 2.38 (s, 3H), 1.69 (d, J = 6.8 Hz, 3H). |
| 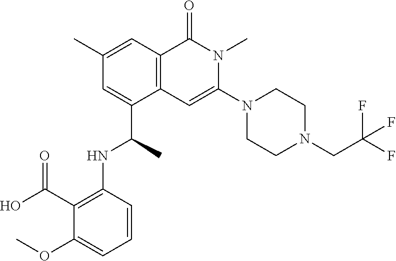 [SMILES: COc1cccc(N[C@H](C)c2cc(C)cc3c(=O)n(C)c(N4CCN(CC(F)(F)F)CC4)cc23)c1C(=O)O] C₂₇H₃₁F₃N₄O₄ | 300 | | ES− 531.3 [M − H] | ¹H NMR (DMSO-d₆, 400 MHz, ppm): δ 12.43 (s, 1H), 7.86 (s, 1H), 7.40 (d, J = 1.9 Hz, 1H), 7.26 (d, J = 6.0 Hz, 1H), 7.15- 7.06 (m, 1H), 6.38 (s, 1H), 6.26 (d, J = 8.2 Hz, 1H), 6.02 (d, J = 8.5 Hz, 1H), 5.14- 5.06 (m, 1H), 3.76 (s, 3H), 3.49 (s, 3H), 3.25-3.20 (m, 2H), 3.15-2.60 (m, 8H), 2.32 (s, 3H), 1.49 (d, J = 6.5 Hz, 3H). |
| 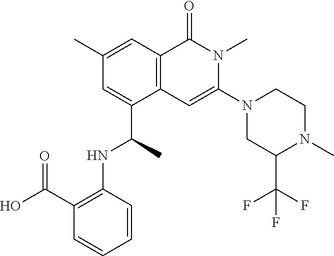 [SMILES: Cc1cc([C@@H](C)Nc2ccccc2C(=O)O)c2cc(N3CCN(C)C(C(F)(F)F)C3)n(C)c(=O)c2c1] C₂₆H₂₉F₃N₄O₃ | 301 | piperazine diastereomer 1 | ES− 501.3 [M − H] | ¹H NMR (400 MHz, DMSO-d₆) δ 12.81 (s, 1H), 8.43 (s, 1H), 7.89 (s, 1H), 7.81 (d, J = 7.9 Hz, 1H), 7.44 (s, 1H), 7.23-7.14 (m, 1H), 6.56-6.48 (m, 1H), 6.47 (s, 1H), 6.39 (d, J = 8.5 Hz, 1H), 5.20-5.10 (m, 1H), 3.50 (s, 3H), 3.31-3.23 (m, 2H), 3.15-2.89 (m, 4H), 2.65-2.60 (m, 1H), 2.50 (s, 3H), 2.33 (s, 3H), 1.54 (d, J = 6.5 Hz, 3H). |
| 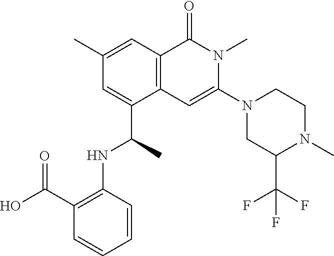 [SMILES: Cc1cc([C@@H](C)Nc2ccccc2C(=O)O)c2cc(N3CCN(C)C(C(F)(F)F)C3)n(C)c(=O)c2c1] C₂₆H₂₉F₃N₄O₃ | 302 | piperazine diastereomer 2 | ES− 501.3 [M − H] | ¹H NMR (400 MHz, DMSO-d₆) δ 12.76 (s, 1H), 8.40 (s, 1H), 7.89 (s, 1H), 7.84- 7.77 (m, 1H), 7.44 (d, J = 2.0 Hz, 1H), 7.24-7.16 (m, 1H), 6.56-6.48 (m, 1H), 6.47-6.39 (m, 2H), 5.24-5.16 (m, 1H), 3.50 (s, 3H), 3.32- 3.28 (m, 2H), 3.19- 2.75 (m, 4H), 2.66- 2.63 (m, 1H), 2.54 (s, 3H), 2.34 (s, 3H), 1.54 (d, J = 6.5 Hz, 3H). |
| 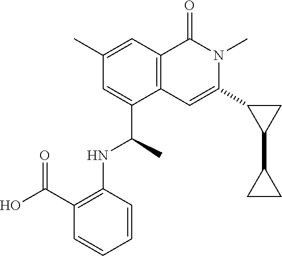 C₂₆H₂₈N₂O₃ | 303 | trans cyclopropyl, mix of diastereomers | ES+ 417.2 [M + H] | ¹H NMR (400 MHz, CDCl₃) δ 8.15 (s, 2H), 8.01 (d, J = 8.0 Hz, 1H), 7.48 (s, 1H), 7.25-7.14 (m, 1H), 6.60 (t, J = 7.6 Hz, 1H), 6.52 (d, J = 2.4 Hz, 1H), 6.28 (t, J = 8.4 Hz, 1H), 5.08- 4.91 (m, 1H), 3.82 (s, 3H), 2.40 (s, 3H), 1.77-1.72 (m, 1H), 1.66-1.60 (m, 3H), 1.15-0.79 (m, 4H), 0.67-0.41 (m, 2H), 0.35-0.11 (m, 2H). |
| 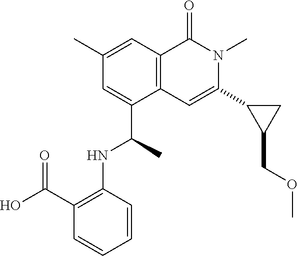 C₂₅H₂₈N₂O₄ | 304 | trans cyclopropyl, mix of diastereomers | ES+ 421.2 [M + H] | ¹H NMR (400 MHz, DMSO-d₆) δ 12.76 (br s, 1H), 8.49- 8.35 (m, 1H), 7.91 (s, 1H), 7.80 (d, J = 8.0 Hz, 1H), 7.43 (s, 1H), 7.17 (t, J = 7.6 Hz, 1H), 6.64 (d, J = 8.4 Hz, 1H), 6.51 (t, J = 7.2 Hz, 1H), 6.35 (d, J = 7.6 Hz, 1H), 5.28-5.12 (m, 1H), 3.68 (s, 3H), 3.43 (t, J = 6.4 Hz, 2H), 3.32 (s, 3H), 2.33 (s, 3H), 2.00-1.95 (m, 1H), 1.50 (d, J = 5.6 Hz, 3H), 1.47-1.41 (m, 1H), 1.23-1.14 (m, 1H), 0.98-0.88 (m, 1H). |
| 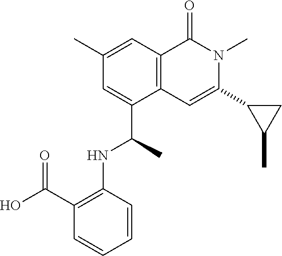 C₂₄H₂₆N₂O₃ | 305 | trans cyclopropyl, mix of diastereomers | ES+ 413.2 [M + Na] | ¹H NMR (400 MHz, CDCl₃) δ 8.15 (s, 2H), 8.02 (d, J = 8.0 Hz, 1H), 7.49 (s, 1H), 7.24-7.15 (m, 1H), 6.65-6.49 (m, 2H), 6.28-6.25 (m, 1H), 5.09-4.92 (m, 1H), 3.79 (s, 3H), 2.40 (s, 3H), 1.70- 1.57 (m, 4H), 1.36- 1.26 (m, 3H), 1.17- 1.01 (m, 2H), 0.87- 0.78 (m, 1H). |
| 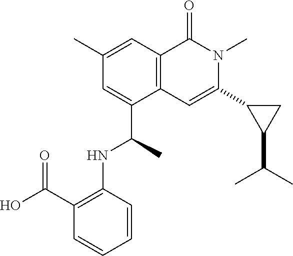 C₂₆H₃₀N₂O₃ | 306 | trans cyclopropyl, mix of diastereomers | ES+ 419.2 [M + H] | ¹H NMR (400 MHz, CDCl₃) δ 8.15 (s, 2H), 8.02 (d, J = 8.0 Hz, 1H), 7.49 (s, 1H), 7.21 (t, J = 7.2 Hz, 1H), 6.68-6.58 (m, 1H), 6.56 (s, 1H), 6.31-6.28 (m, 1H), 5.09-4.92 (m, 1H), 3.81 (s, 3H), 2.40 (s, 3H), 1.75- 1.69 (m, 1H), 1.66 (d, J = 6.4 Hz, 3H), 1.32-1.30 (m, 1H), 1.20-1.08 (m, 4H), 1.05-1.03 (m, 3H), 0.94-0.81 (m, 2H). |
| 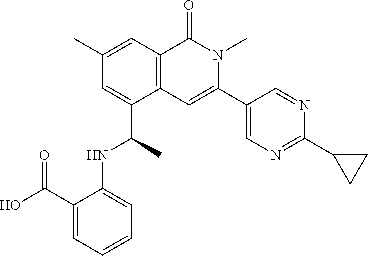 [SMILES: Cc1cc([C@@H](C)Nc2ccccc2C(=O)O)c2cc(-c3cnc(C4CC4)nc3)n(C)c(=O)c2c1] C₂₇H₂₆N₄O₃ | 307 | | ES− 453.2 [M − H] | ¹H NMR (400 MHz, DMSO-d₆) δ 12.76 (s, 1H), 8.91 (d, J = 2.7 Hz, 2H), 8.41 (s, 1H), 8.00 (s, 1H), 7.84-7.77 (m, 1H), 7.50 (s, 1H), 7.19- 7.04 (m, 1H), 7.03 (s, 1H), 6.55-6.49 (m, 1H), 6.35 (d, J = 8.5 Hz, 1H), 5.26- 5.20 (m, 1H), 3.38 (s, 3H), 2.31 (s, 3H), 2.30-2.20 (m, 1H), 1.51 (d, J = 6.5 Hz, 3H), 1.17-1.07 (m, 4H) |
| 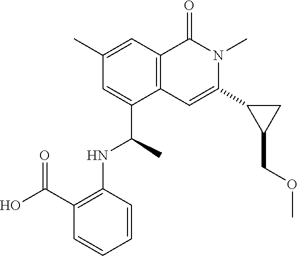 C₂₅H₂₈N₂O₄ | 308 | trans cyclopropyl diastereomer 1 | ES+ 421.3 [M + H] | ¹H NMR (400 MHz, DMSO-d₆) δ 9.19- 8.70 (m, 1H), 7.89 (s, 1H), 7.82 (d, J = 7.2 Hz, 1H), 7.45 (s, 1H), 7.06 (t, J = 7.2 Hz, 1H), 6.64 (s, 1H), 6.45 (t, J = 7.6 Hz, 1H), 6.26 (d, J = 8.4 Hz, 1H), 5.21- 5.01 (m, 1H), 3.68 (s, 3H), 3.45-3.39 (m, 2H), 3.29 (s, 3H), 2.32 (s, 3H), 2.04-1.91 (m, 1H), 1.48 (d, J = 6.4 Hz, 3H), 1.46-1.40 (m, 1H), 1.22-1.16 (m, 1H), 0.91-0.89 (m, 1H). |
| 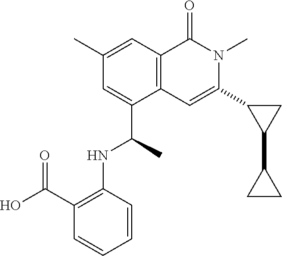 C₂₆H₂₈N₂O₃ | 309 | trans cyclopropyl diastereomer 1 | ES+ 439.2 [M + Na] | ¹H NMR (400 MHz, CDCl₃) δ 8.25-8.07 (m, 2H), 7.98 (d, J = 7.2 Hz, 1H), 7.47 (s, 1H), 7.24-7.09 (m, 1H), 6.58-6.55 (m, 1H), 6.50 (s, 1H), 6.25 (d, J = 8.4 Hz, 1H), 5.06-4.89 (m, 1H), 3.81 (s, 3H), 2.37 (s, 3H), 1.74- 1.71 (m, 1H), 1.62 (d, J = 5.2 Hz, 3H), 1.14-1.04 (m, 1H), 0.99-0.90 (m, 2H), 0.84-0.79 (m, 1H), 0.61-0.45 (m, 2H), 0.30-0.17 (m, 2H). |
| 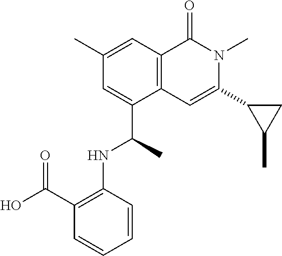 C₂₄H₂₆N₂O₃ | 310 | trans cyclopropyl diastereomer 1 | ES+ 413.2 [M + Na] | ¹H NMR (400 MHz, CDCl₃) δ 8.29-8.09 (m, 2H), 8.00-7.95 (m, 1H), 7.48 (s, 1H), 7.24-7.15 (m, 1H), 6.67-6.49 (m, 2H), 6.27 (d, J = 8.4 Hz, 1H), 5.09-4.94 (m, 1H), 3.78 (s, 3H), 2.40 (s, 3H), 1.68-1.61 (m, 4H), 1.32 (d, J = 6.0 Hz, 3H), 1.16-1.01 (m, 2H), 0.86-0.80 (m, 1H). |
| 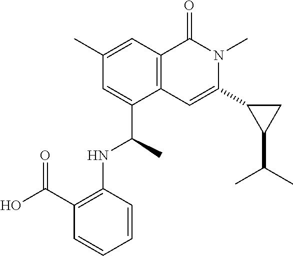 C₂₆H₃₀N₂O₃ | 311 | trans cyclopropyl diastereomer 1 | ES+ 419.3 [M + H] | ¹H NMR (400 MHz, CDCl₃) δ 8.14 (s, 2H), 7.99 (d, J = 6.8 Hz, 1H), 7.48 (s, 1H), 7.21 (t, J = 7.6 Hz, 1H), 6.66-6.50 (m, 2H), 6.31 (d, J = 8.4 Hz, 1H), 4.99- 4.90 (m, 1H), 3.80 (s, 3H), 2.40 (s, 3H), 1.74-1.69 (m, 1H), 1.65 (d, J = 6.8 Hz, 3H), 1.36-1.30 (m, 1H), 1.13-1.04 (m, 6H), 0.93-0.80 (m, 3H). |
| 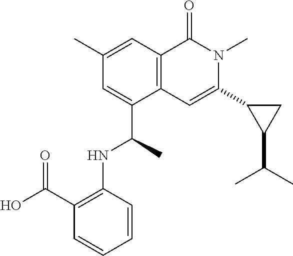 C₂₆H₃₀N₂O₃ | 312 | trans cyclopropyl diastereomer 2 | ES+ 419.3 [M + H] | ¹H NMR (400 MHz, CDCl₃) δ 8.14 (s, 2H), 7.93 (d, J = 7.6 Hz, 1H), 7.48 (s, 1H), 7.21 (t, J = 7.6 Hz, 1H), 6.65-6.51 (m, 2H), 6.31 (d, J = 8.4 Hz, 1H), 5.07- 4.93 (m, 1H), 3.80 (s, 3H), 2.40 (s, 3H), 1.75-1.68 (m, 1H), 1.65 (d, J = 6.8 Hz, 3H), 1.34-1.27 (m, 1H), 1.13-1.03 (m, 6H), 0.88-0.80 (m, 3H). |
| 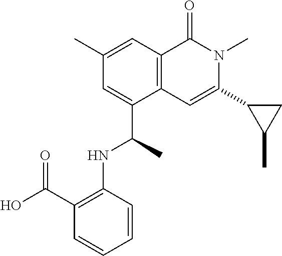 C₂₄H₂₆N₂O₃ | 313 | trans cyclopropyl diastereomer 2 | ES+ 413.2 [M + Na] | ¹H NMR (400 MHz, CDCl₃) δ 8.14 (s, 2H), 8.00-7.98 (m, 1H), 7.48 (s, 1H), 7.23-7.15 (m, 1H), 6.64-6.52 (m, 2H), 6.28 (d, J = 8.4 Hz, 1H), 5.08-4.96 (m, 1H), 3.78 (s, 3H), 2.40 (s, 3H), 1.65- 1.60 (m, 4H), 1.31 (d, J = 5.6 Hz, 3H), 1.13-1.04 (m, 2H), 0.83 (d, J = 8.4 Hz, 1H). |
| 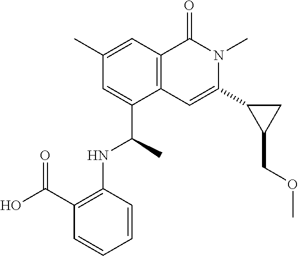 C₂₅H₂₈N₂O₄ | 314 | trans cyclopropyl diastereomer 2 | ES+ 421.3 [M + H] | ¹H NMR (400 MHz, DMSO-d₆) δ 8.86 (br s, 1H), 7.90 (s, 1H), 7.81 (d, J = 7.6 Hz, 1H), 7.45 (s, 1H), 7.15-6.98 (m, 1H), 6.66 (s, 1H), 6.46 (t, J = 7.6 Hz, 1H), 6.28 (d, J = 8.4 Hz, 1H), 5.22-5.0 (m, 1H), 3.68 (s, 3H), 3.48-3.40 (m, 2H), 3.30 (s, 3H), 2.33 (s, 3H), 2.03-1.97 (m, 1H), 1.48 (d, J = 6.4 Hz, 3H), 1.46-1.39 (m, 1H), 1.22-1.16 (m, 1H), 0.97-0.88 (m, 1H). |
| 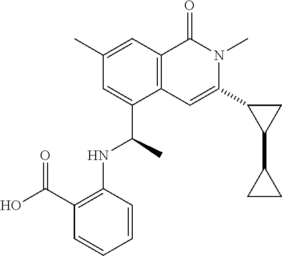 C₂₆H₂₈N₂O₃ | 315 | trans cyclopropyl diastereomer 2 | ES+ 439.2 [M + Na] | ¹H NMR (400 MHz, CDCl₃) δ 8.31-8.07 (m, 2H), 8.06-7.88 (m, 1H), 7.47 (s, 1H), 7.23-7.08 (m, 1H), 6.62-6.53 (m, 2H), 6.30-6.25 (m, 1H), 5.11-4.82 (m, 1H), 3.80 (s, 3H), 2.36 (s, 3H), 1.77- 1.71 (m, 1H), 1.62- 1.52 (m, 3H), 1.05 (m, 1H), 0.99-0.91 (m, 2H), 0.86-0.80 (m, 1H), 0.61-0.44 (m, 2H), 0.29-0.16 (m, 2H). |
| 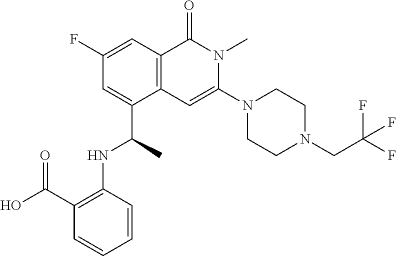 [SMILES: C[C@@H](Nc1ccccc1C(=O)O)c1cc(F)cc2c(=O)n(C)c(N3CCN(CC(F)(F)F)CC3)cc12] C₂₅H₂₆F₄N₄O₃ | 316 | | ES− 505.2 [M − H] | ¹H NMR (400 MHz, DMSO-d₆) δ 8.61- 8.53 (m, 1H),7.81 (d, J = 7.8 Hz, 1H), 7.73-7.66 (m, 1H), 7.41-7.33 (m, 1H), 7.14 (d, J = 8.2 Hz, 1H), 6.56-6.48 (m, 1H), 6.46 (s, 1H), 6.32 (d, J = 8.6 Hz, 1H), 5.23 (s, 1H), 3.51 (s, 3H), 3.27 (d, J = 10.2 Hz, 2H), 3.05-2.53 (m, 8H), 1.54 (d, J = 6.5 Hz, 3H) |
| 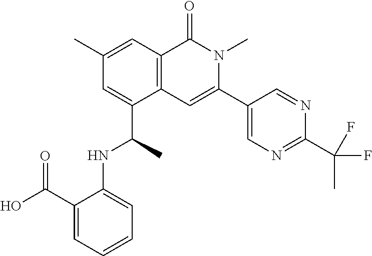 [SMILES: Cc1cc([C@@H](C)Nc2ccccc2C(=O)O)c2cc(-c3cnc(C(C)(F)F)nc3)n(C)c(=O)c2c1] C₂₆H₂₄F₂N₄O₃ | 317 | | ES− 477.2 [M − H] | ¹H NMR (400 MHz, Methanol-d₄) δ 9.15 (d, J = 2.7 Hz, 2H), 8.12 (s, 1H), 7.94- 7.88 (m, 1H), 7.65 (s, 1H), 7.17-7.06 (m, 2H), 6.60-6.52 (m, 1H), 6.34 (d, J = 8.4 Hz, 1H), 5.20- 5.10 (m, 1H), 3.54- 3.48 (m, 3H), 2.44 (d, J = 4.8 Hz, 3H), 2.19-2.07 (m, 3H), 1.62-1.61 (m, 3H). |
| 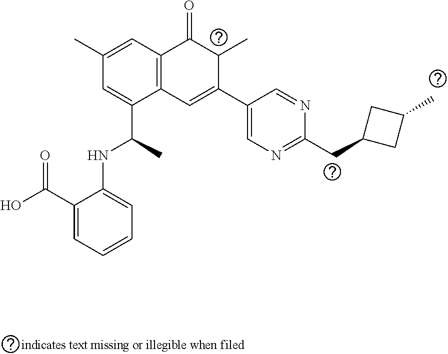 C₂₈H₂₇FN₄O₄ | 318 | | ES+ 503.1 [M + H] | ¹H NMR (400 MHz, DMSO-d₆) δ 12.75 (s, 1H), 8.89 (s, 2H), 8.39 (d, J = 6.1 Hz, 1H), 8.00 (d, J = 1.9 Hz, 1H), 7.82-7.78 (m, 1H), 7.49 (d, J = 1.9 Hz, 1H), 7.20- 7.14 (m, 1H), 7.02 (s, 1H), 6.57-6.48 (m, 1H), 6.35 (d, J = 8.5 Hz, 1H), 5.31- 5.22 (m, 1H), 5.03- 4.88 (m, 2H), 3.39 (s, 3H), 3.11-2.98 (m, 2H), 2.41-2.31 (m, 5H), 1.51 (d, J = 6.5 Hz, 3H). |
| 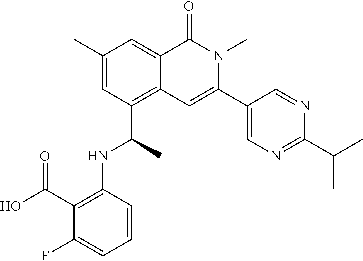 [SMILES: Cc1cc([C@@H](C)Nc2cccc(F)c2C(=O)O)c2cc(-c3cnc(C(C)C)nc3)n(C)c(=O)c2c1] C₂₇H₂₇FN₄O₃ | 319 | | ES− 473.0 [M − H] | ¹H NMR (DMSO-d₆, 400 MHz, ppm): δ 13.17 (s, 1H), 9.03 (s, 2H), 8.15 (d, J = 5.9 Hz, 1H), 8.04- 7.99 (m, 1H), 7.48 (d, J = 1.9 Hz, 1H), 7.20-7.10 (m, 1H), 7.05 (s, 1H), 6.42- 6.32 (m, 1H), 6.14 (d, J = 8.6 Hz, 1H), 5.30-5.22 (m, 1H), 3.40 (s, 3H), 3.24- 3.19 (m, 1H), 2.39 (s, 3H), 1.49 (d, J = 6.5 Hz, 3H), 1.35 (d, J = 6.9 Hz, 6H). |
| 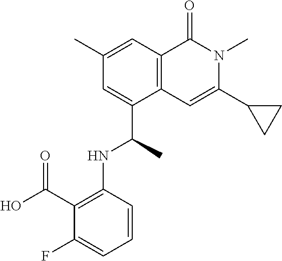 [SMILES: Cc1cc([C@@H](C)Nc2cccc(F)c2C(=O)O)c2cc(C3CC3)n(C)c(=O)c2c1] C₂₃H₂₃FN₂O₃ | 320 | | ES+ 417.2 [M + Na] | ¹H NMR (400 MHz, DMSO-d₆) δ 8.16- 8.09 (m, 1H), 7.92 (s, 1H), 7.42 (s, 1H), 7.20-7.07 (m, 1H), 6.62 (s, 1H), 6.35- 6.26 (m, 1H), 6.15 (d, J = 8.4 Hz, 1H), 5.25-5.10 (m, 1H), 3.68 (s, 3H), 2.34 (s, 3H), 2.11-1.96 (m, 1H), 1.49 (d, J = 6.4 Hz, 3H), 1.04-0.94 (m, 2H), 0.90-0.78 (m, 2H). |
| 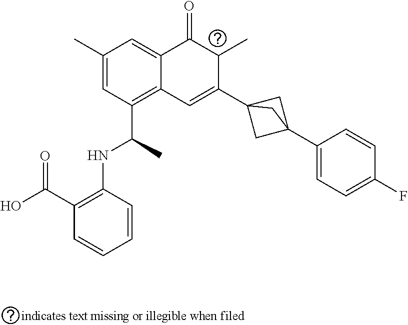 [SMILES: Cc1cc2c(c([C@@H](C)Nc3ccccc3C(=O)O)c1)C=C(C13CC(c4ccc(F)cc4)(C1)C3)C(C)C2=O] C₃₁H₂₉FN₂O₃ | 321 | | ES+ 519.3 [M + Na] | ¹H NMR (400 MHz, CDCl₃) 8 8.21-8.15 (m, 2H), 8.03- 7.97 (m, 1H), 7.55-7.50 (m, 1H), 7.28-7.20 (m, 3H), 7.07-7.00 (m, 2H), 6.69-6.57 (m, 2H), 6.38- 6.31 (m, 1H), 5.15-4.96 (m, 1H), 3.80 (s, 3H), 2.54 (s, 6H), 2.43 (s, 3H), 1.69 (d, J = 6.8 Hz, 3H). |
| 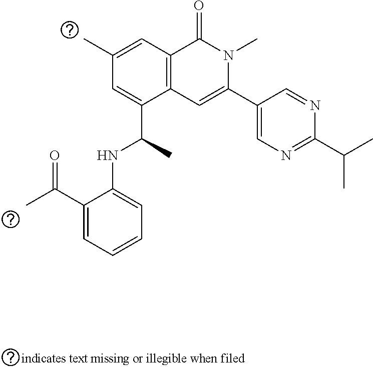 [SMILES: CC(=O)c1ccccc1N[C@H](C)c1cc(C)cc2c(=O)n(C)c(-c3cnc(C(C)C)nc3)cc12] C₂₆H₂₅FN₄O₃ | 322 | | ES+ 461.0 [M + H] | ¹H NMR (DMSO-d₆, 400 MHz, ppm): δ 12.83 (s, 1H), 9.04 (s, 2H), 8.42 (s, 1H), 7.94-7.83 (m, 2H), 7.55-7.44 (m, 1H), 7.22- 7.13 (m, 1H), 7.13 (s, 1H), 6.65-6.55 (m, 1H), 6.30 (d, J = 8.5 Hz, 1H), 5.35 (s, 1H), 3.40 (s, 3H), 3.30-3.25 (m, 1H), 1.53 (d, J = 6.5 Hz, 3H), 1.36 (d, J = 6.9 Hz, 6H). |
| 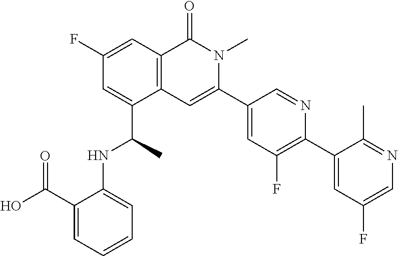 [SMILES: Cc1ncc(F)cc1-c1ncc(-c2cc3c([C@@H](C)Nc4ccccc4C(=O)O)cc(F)cc3c(=O)n2C)cc1F] C₃₀H₂₃F₃N₄O₃ | 323 | | ES+ 545.4 [M + H] | ¹H NMR (400 MHz, DMSO-d₆) δ 12.79 (s, 1H), 8.90-8.85 (m, 1H), 8.64 (d, J = 2.9 Hz, 1H), 8.43 (s, 1H), 8.37-8.30 (m, 1H), 7.92-7.86 (m, 2H), 7.86- 7.80 (m, 1H), 7.50- 7.43 (m, 1H), 7.24-7.14 (m, 2H), 6.60-6.52 (m, 1H), 6.34 (d, J = 8.5 Hz, 1H), 5.37-5.35 (m, 1H), 3.46 (s, 3H), 2.47 (s, 3H), 1.56 (d, J = 6.6 Hz, 3H). |
| 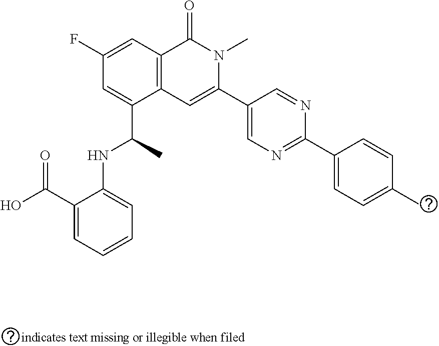 C₂₉H₂₂F₂N₄O₃ | 324 | | ES+ 513.3 [M + H] | ¹H NMR (400 MHz, DMSO-d₆) δ 9.21 (s, 2H), 8.60-8.50 (m, 3H), 7.90- 7.79 (m, 2H), 7.50- 7.38 (m, 3H), 7.21 (s, 1H), 7.18-7.14 (m, 1H), 6.56- 6.52 (m, 1H), 6.31 (d, J = 8.5 Hz, 1H), 5.34-5.28 (m, 1H), 3.47 (s, 3H), 1.55 (d, J = 6.5 Hz, 3H). |
| 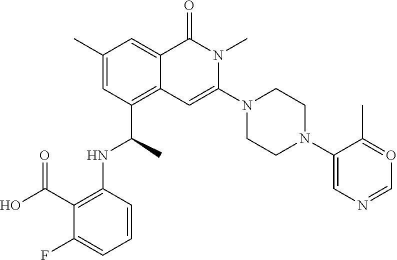 C₂₉H₃₁FN₆O₃ | 325 | | ES+ 531.5 [M + H] | ¹H NMR (Methanol-d₄, 400 MHz, ppm): δ 8.74 (s, 1H), 8.50 (s, 1H), 8.02 (s, 1H), 7.59 (s, 1H), 7.11- 6.98 (m, 1H), 6.60 (s, 1H), 6.36-6.24 (m, 1H), 6.19 (d, J = 8.5 Hz, 1H), 5.21- 5.03 (m, 1H), 3.71 (s, 3H), 3.31-3.19 (m, 6H), 3.18- 3.02 (m, 2H), 2.61 (s, 3H), 2.41 (s, 3H), 1.66 (d, J = 6.6 Hz, 3H). |
| 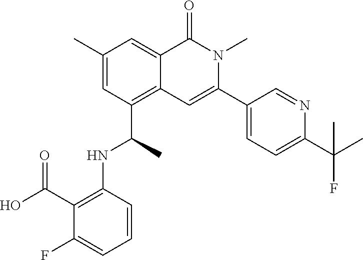 [SMILES: Cc1cc([C@@H](C)Nc2cccc(F)c2C(=O)O)c2cc(-c3ccc(C(C)(C)F)nc3)n(C)c(=O)c2c1] C₂₈H₂₇F₂N₃O₃ | 326 | | ES+ 492.1 [M + H] | ¹H NMR (400 MHz, Methanol-d₄) δ 8.76- 8.70 (m, 1H), 8.12 (s, 1H), 8.09-8.02 (m, 1H), 7.77 (d, J = 8.1 Hz, 1H), 7.63 (d, J = 1.8 Hz, 1H), 7.08- 6.98 (m, 1H), 6.99 (s, 1H), 6.31-6.22 (m, 1H), 6.10 (d, J = 8.5 Hz, 1H), 5.19- 5.09 (m, 1H), 3.48 (s, 3H), 2.44 (s, 3H), 1.79 (s, 3H), 1.74 (s, 3H), 1.60 (d, J = 6.7 Hz, 3H). |
| 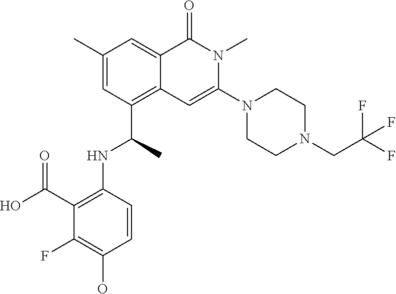 C₂₆H₂₇ClF₄N₄O₃ | 327 | | ES+ 555.1 [M + H] | ¹H NMR (400 MHz, DMSO-d₆) δ 13.56 (s, 1H), 8.09 (d, J = 5.9 Hz, 1H), 7.89 (s, 1H), 7.44-7.29 (m, 2H), 6.38 (s, 1H), 6.22 (d, J = 9.3 Hz, 1H), 5.18- 5.15 (m, 1H), 3.50 (s, 3H), 3.31-3.25 (m, 2H), 3.18- 2.72 (m, 8H), 2.34 (s, 3H), 1.53 (d, J = 6.5 Hz, 3H). |
| 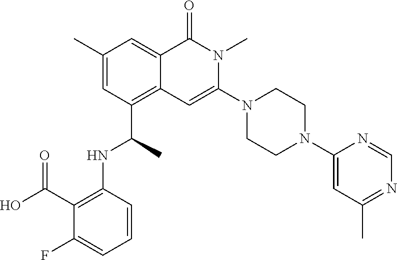 [SMILES: Cc1cc([C@@H](C)Nc2cccc(F)c2C(=O)O)c2cc(N3CCN(c4cc(C)ncn4)CC3)n(C)c(=O)c2c1] C₂₉H₃₁FN₆O₃ | 328 | | ES+ 531.3 [M + H] | ¹H NMR (Chloroform-d, 400 MHz, ppm): δ 8.83- 8.50 (m, 2H), 8.15 (s, 1H), 7.47 (s, 1H), 7.16-6.95 (m, 1H), 6.69 (s, 1H), 6.42 (s, 1H), 6.35-6.23 (m, 1H), 6.19 (d, J = 8.7 Hz, 1H), 4.84 (d, J = 7.1 Hz, 1H), 4.72-4.19 (m, 2H), 3.65 (s, 3H), 3.48-2.73 (m, 6H), 2.48 (s, 6H), 1.73 (d, J = 6.8 Hz, 3H). |
| 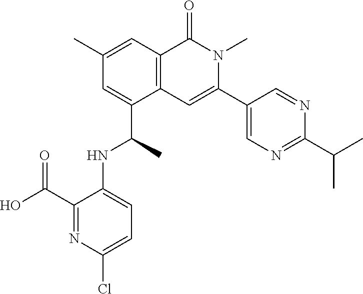 [SMILES: Cc1cc([C@@H](C)Nc2ccc(Cl)nc2C(=O)O)c2cc(-c3cnc(C(C)C)nc3)n(C)c(=O)c2c1] C₂₆H₂₆ClN₅O₃ | 329 | | ES+ 492.4 [M + H] | ¹H NMR (400 MHz, Methanol-d₄) δ 8.96 (s, 2H), 8.12 (s, 1H), 7.61 (d, J = 1.8 Hz, 1H), 7.13 (d, J = 8.9 Hz, 1H), 7.05 (s, 1H), 6.85 (d, J = 8.9 Hz, 1H), 5.27-5.18 (m, 1H), 3.50 (s, 3H), 3.29-3.20 (m, 1H), 2.44 (s, 3H), 1.64 (d, J = 6.6 Hz, 3H), 1.42 (d, J = 6.9 Hz, 6H). |
| 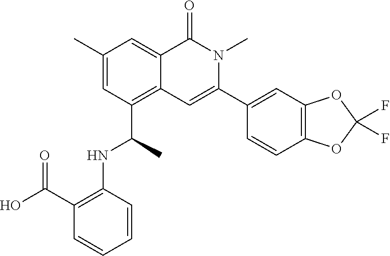 [SMILES: Cc1cc([C@@H](C)Nc2ccccc2C(=O)O)c2cc(-c3ccc4c(c3)OC(F)(F)O4)n(C)c(=O)c2c1] C₂₇H₂₂F₂N₂05 | 330 | | ES+ 493.1 [M + H] | ¹H NMR (400 MHz, DMSO-d₆) δ 12.65 (s, 1H), 8.57 (s, 1H), 7.89 (d, J = 1.9 Hz, 1H), 7.81-7.75 (m, 2H), 7.56 (d, J = 8.3 Hz, 1H), 7.49-7.45 (m, 2H), 7.15-7.11 (m, 1H), 6.88 (s, 1H), 6.52-6.48 (m, 1H), 6.29 (d, J = 8.4 Hz, 1H), 5.20-5.18 (m, 1H), 3.34 (s, 3H), 2.37 (s, 3H), 1.51 (d, J = 6.5 Hz, 3H) |
| 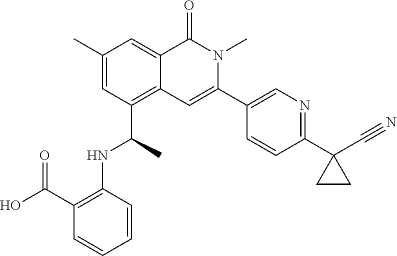 [SMILES: Cc1cc([C@@H](C)Nc2ccccc2C(=O)O)c2cc(-c3ccc(C4(C#N)CC4)nc3)n(C)c(=O)c2c1] C₂₉H₂₆N₄O₃ | 331 | | ES− 477.4 [M − H] | ¹H NMR (400 MHz, Methanol-d₄) δ 8.67 (d, J = 2.3 Hz, 1H), 8.10 (s, 1H), 8.03-7.97 (m, 1H), 7.91 (d, J = 7.8 Hz, 1H), 7.83 (d, J = 8.2 Hz, 1H), 7.64 (s, 1H), 7.14-7.05 (m, 1H), 6.99 (s, 1H), 6.56-6.43 (m, 1H), 6.31 (d, J = 8.4 Hz, 1H), 5.15-5.10 (m, 1H), 3.46 (s, 3H), 2.43 (s, 3H), 1.94-1.84 (m, 4H), 1.62 (d, J = 6.7 Hz, 3H). |
| 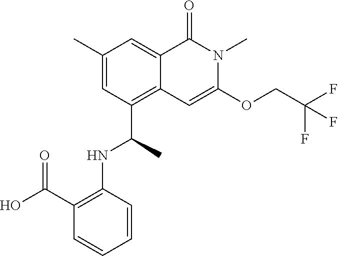 [SMILES: Cc1cc([C@@H](C)Nc2ccccc2C(=O)O)c2cc(OCC(F)(F)F)n(C)c(=O)c2c1] C₂₂H₂₁F₃N₂O₄ | 332 | | ES− 433.1 [M − H] | ¹H NMR (400 MHz, DMSO-d₆) δ 12.83 (s, 1H), 8.55 (s, 1H), 7.89-7.84 (m, 1H), 7.82-7.69 (m, 1H), 7.41 (d, J = 1.9 Hz, 1H), 7.16-7.05 (m, 1H), 6.55-6.46 (m, 1H), 6.44 (s, 1H), 6.30 (d, J = 8.4 Hz, 1H), 5.20-5.10 (m, 1H), 5.16-4.97 (m, 2H), 3.46 (s, 3H), 2.30 (s, 3H), 1.51 (d, J = 6.5 Hz, 3H). |
| 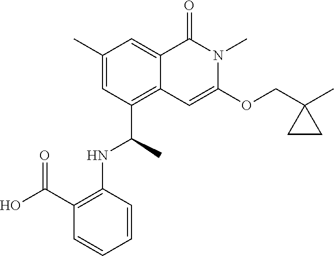 [SMILES: Cc1cc([C@@H](C)Nc2ccccc2C(=O)O)c2cc(OCC3(C)CC3)n(C)c(=O)c2c1] C₂₅H₂₈N₂O₄ | 333 | | ES− 419.2 [M − H] | ¹H NMR (400 MHz, DMSO-d₆) δ 12.73 (s, 1H), 8.36 (d, J = 5.9 Hz, 1H), 7.86-7.76 (m, 2H), 7.37 (d, J = 1.9 Hz, 1H), 7.20- 7.15 (m, 1H), 6.55-6.45 (m, 1H), 6.34 (d, J = 8.5 Hz, 1H), 6.12 (s, 1H), 5.18- 5.00 (m, 1H), 4.02- 3.91 (m, 2H), 3.48 (s, 3H), 2.29 (s, 3H), 1.51 (d, J = 6.6 Hz, 3H), 1.24 (s, 3H), 0.61-0.55 (m, 2H), 0.50- 0.42 (m, 2H). |
| 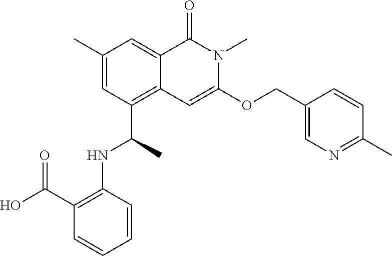 [SMILES: Cc1cc([C@@H](C)Nc2ccccc2C(=O)O)c2cc(OCc3ccc(C)nc3)n(C)c(=O)c2c1] C₂₇H₂₇N₃O₄ | 334 | | ES+ 458.3 [M + H] | ¹H NMR (400 MHz, DMSO-d₆) δ 12.77 (s, 1H), 8.64 (d, J = 2.3 Hz, 1H), 7.91-7.82 (m, 3H), 7.81- 7.78 (m, 1H), 7.40 (d, J = 1.9 Hz, 1H), 7.32 (d, J = 7.9 Hz, 1H), 7.13 (s, 1H), 6.57-6.46 (m, 1H), 6.37 (s, 1H), 6.29-6.27 (m, 1H), 5.38-5.30 (m, 2H), 5.16-5.10 (m, 1H), 3.45 (s, 3H), 2.50 (s, 3H) 2.30 (s, 3H), 1.50 (d, J = 6.5 Hz, 3H). |
| 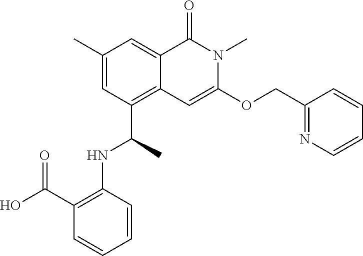 [SMILES: Cc1cc([C@@H](C)Nc2ccccc2C(=O)O)c2cc(OCc3ccccn3)n(C)c(=O)c2c1] C₂₆H₂₅N₃O₄ | 335 | | ES− 442.3 [M − H] | ¹H NMR (400 MHz, DMSO-d₆) δ 12.72 (s, 1H), 8.61 (d, J = 5.0 Hz, 1H), 8.42 (s, 1H), 7.91-7.79 (m, 3H), 7.66 (d, J = 8.0 Hz, 1H), 7.40-7.37 (m, 2H), 7.17-7.13 (m, 1H), 6.53-6.50 (m, 1H), 6.35- 6.28 (m, 2H), 5.49- 5.43 (m, 2H), 5.13-5.11 (m, 1H), 3.54 (s, 3H), 1.46 (d, J = 6.5 Hz, 3H), 0.52- 0.33 (m, 3H). |
| 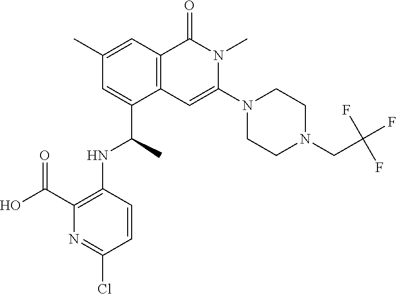 [SMILES: Cc1cc([C@@H](C)Nc2ccc(Cl)nc2C(=O)O)c2cc(N3CCN(CC(F)(F)F)CC3)n(C)c(=O)c2c1] C₂₅H₂₇ClF₃N₅O₃ | 336 | | ES− 536.2 [M − H] | ¹H NMR (400 MHz, DMSO-d₆) δ 8.75 (s, 1H), 7.88 (s, 1H), 7.40 (s, 1H), 7.24 (d, J = 8.7 Hz, 1H), 6.84 (d, J = 8.9 Hz, 1H), 6.39 (s, 1H), 5.19-5.10 (m, 1H), 3.49 (s, 3H), 3.25- 3.15 (m, 2H), 3.14- 2.75 (m, 8H), 2.32 (s, 3H), 1.53 (d, J = 6.5 Hz, 3H). |
| 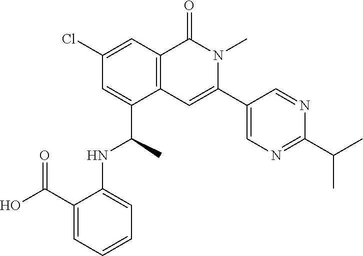 [SMILES: CC(C)c1ncc(-c2cc3c([C@@H](C)Nc4ccccc4C(=O)O)cc(Cl)cc3c(=O)n2C)cn1] C₂₆H₂₅ClN₄O₃ | 337 | | ES− 475.2 [M − H] | ¹H NMR (400 MHz, Methanol-d₄) δ 8.96 (s, 2H), 8.24 (d, J = 2.2 Hz, 1H), 7.93-7.90 (m, 1H), 7.70 (d, J = 2.2 Hz, 1H), 7.13-7.10 (m, 1H), 7.08 (s, 1H), 6.56 (d, J = 7.5 Hz, 1H), 6.29 (d, J = 8.5 Hz, 1H), 5.20-5.10 (m, 1H), 3.50 (s, 3H), 3.32-3.28 (m, 1H), 1.62 (d, J = 6.6 Hz, 3H), 1.42 (d, J = 6.9 Hz, 6H). |
| 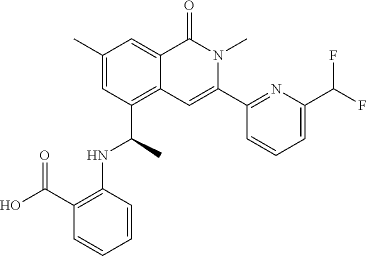 [SMILES: Cc1cc([C@@H](C)Nc2ccccc2C(=O)O)c2cc(-c3cccc(C(F)F)n3)n(C)c(=O)c2c1] C₂₆H₂₃F₂N₃O₃ | 338 | | ES− 462.1 [M − H] | ¹H NMR (400 MHz, DMSO-d₆) δ 12.77 (s, 1H), 8.42 (s, 1H), 8.28-8.19 (m, 1H), 8.03 (s, 1H), 7.98 (d, J = 7.9 Hz, 1H), 7.87- 7.78 (m, 2H), 7.52 (s, 1H), 7.23-7.13 (m, 1H), 7.11 (s, 1H), 7.07 (s, 1H), 6.57- 6.48 (m, 1H), 6.36 (d, J = 8.5 Hz, 1H), 5.31-5.23 (m, 1H), 3.45 (s, 3H), 2.40 (s, 3H), 1.53 (d, J = 6.5 Hz, 3H). |
| 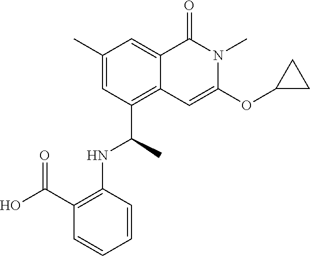 [SMILES: Cc1cc([C@@H](C)Nc2ccccc2C(=O)O)c2cc(OC3CC3)n(C)c(=O)c2c1] C₂₃H₂₄N₂O₄ | 339 | | ES− 391.2 [M − H] | ¹H NMR (400 MHz, DMSO-d₆) δ 12.72 (s, 1H), 8.39 (s, 1H), 7.86 (s, 1H), 7.80 (d, J = 7.8 Hz, 1H), 7.44 (s, 1H), 7.19 (d, 7.8 Hz, 1H), 6.55 (d, J = 7.5 Hz, 1H), 6.46-6.43 (m, 2H), 5.10-5.00 (m, 1H), 4.06-4.03 (m, 1H), 3.31 (s, 3H), 2.33 (s, 3H), 1.57 (d, J = 6.5 Hz, 3H), 0.89- 0.73 (m, 4H). |
| 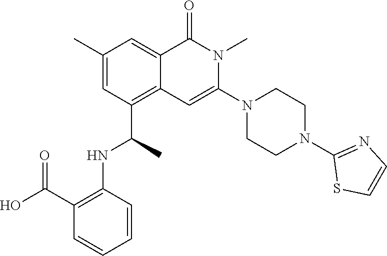 [SMILES: Cc1cc([C@@H](C)Nc2ccccc2C(=O)O)c2cc(N3CCN(c4nccs4)CC3)n(C)c(=O)c2c1] C₂₇H₂₉N₅O₃S | 340 | | ES− 502.2 [M − H] | ¹H NMR (400 MHz, Methanol-d₄) δ 8.01 (s, 1H), 7.91 (d, J = 8.0 Hz, 1H), 7.57 (s, 1H), 7.21 (d, J = 3.7 Hz, 1H), 7.20-7.15 (m, 1H), 6.82 (d, J = 3.7 Hz, 1H), 6.58 (s, 1H), 6.55- 6.50 (m, 1H), 6.42 (d, J = 8.5 Hz, 1H), 5.20-5.10 (m, 1H), 4.15-3.97 (m, 2H), 3.70 (s, 3H), 3.37- 3.31 (m, 4H), 2.99-2.89 (m, 2H), 2.40 (s, 3H), 1.66 (d, J = 6.6 Hz, 3H). |
| 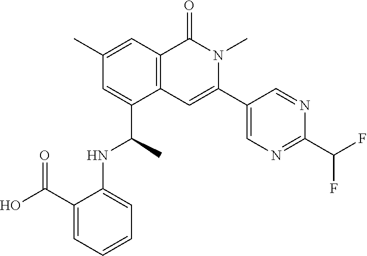 [SMILES: Cc1cc([C@@H](C)Nc2ccccc2C(=O)O)c2cc(-c3cnc(C(F)F)nc3)n(C)c(=O)c2c1] C₂₅H₂₂F₂N₄O₃ | 341 | | ES+ 465.0 [M + H] | ¹H NMR (400 MHz, DMSO-d₆) δ 12.79 (s, 1H), 9.30 (s, 2H), 8.48 (s, 1H), 8.03 (s, 1H), 7.81 (d, J = 7.9 Hz, 1H), 7.52 (s, 1H), 7.20-6.98 (m, 3H), 6.55- 6.51 (m, 1H), 6.34 (d, J = 8.5 Hz, 1H), 5.30-5.23 (m, 1H), 3.42 (s, 3H), 2.39 (s, 3H), 1.52 (d, J = 6.5 Hz, 3H). |
| 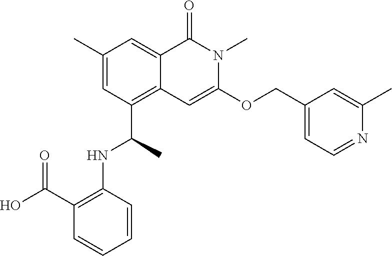 [SMILES: Cc1cc([C@@H](C)Nc2ccccc2C(=O)O)c2cc(OCc3ccnc(C)c3)n(C)c(=O)c2c1] C₂₇H₂₇N₃O₄ | 342 | | ES+ 458.1 [M + H] | ¹H NMR (400 MHz, DMSO-d₆) δ 12.76 (s, 1H), 8.48 (d, J = 5.0 Hz, 1H), 8.44 (s, 1H), 7.85 (d, J = 1.7 Hz, 1H), 7.80-7.78 (m, 1H), 7.42-7.36 (m, 2H), 7.33 (d, J = 5.0 Hz, 1H), 7.15-7.10 (m, 1H), 6.52-6.48 (m, 1H), 6.29- 6.20 (m, 2H), 5.41- 5.25 (m, 1H), 5.30-5.25 (m, 1H), 5.17-5.09 (m, 1H), 3.54 (s, 3H), 2.50 (s, 3H), 2.30 (s, 3H), 1.45 (d, J = 6.5 Hz, 3H). |
| 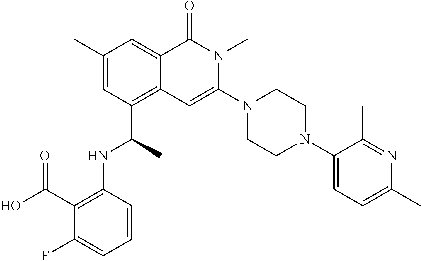 [SMILES: Cc1cc([C@@H](C)Nc2cccc(F)c2C(=O)O)c2cc(N3CCN(c4ccc(C)nc4C)CC3)n(C)c(=O)c2c1] C₃₁H₃₄FN₅O₃ | 343 | | ES+ 544.2 [M + H] | ¹H NMR (400 MHz, Methanol-d₄) δ 8.01 (d, J = 1.9 Hz, 1H), 7.59-7.58 (m, 2H), 7.18 (d, J = 8.2 Hz, 1H), 7.10-7.00 (m, 1H), 6.59 (s, 1H), 6.32- 6.23 (m, 1H), 6.18 (d, J = 8.5 Hz, 1H), 5.16-5.11 (m, 1H), 3.70 (d, J = 1.0 Hz, 3H), 3.31-3.13 (m, 8H), 2.57 (s, 3H), 2.49 (s, 3H), 2.41 (s, 3H), 1.65 (d, J = 6.6 Hz, 3H). |
| 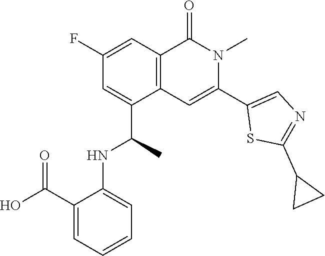 [SMILES: C[C@@H](Nc1ccccc1C(=O)O)c1cc(F)cc2c(=O)n(C)c(-c3cnc(C4CC4)s3)cc12] C₂₅H₂₂FN₃O₃S | 344 | | ES− 462.1 [M − H] | ¹H NMR (400 MHz, DMSO-d₆) δ 12.85 (s, 1H), 8.42 (s, 1H), 7.90 (s, 1H), 7.86-7.79 (m, 2H), 7.46- 7.43 (m, 1H), 7.22- 7.12 (m, 2H), 6.57-6.53 (m, 1H), 6.30 (d, J = 8.5 Hz, 1H), 5.29 (s, 1H), 3.32 (s, 3H), 2.67-2.46 (m, 1H), 1.53 (d, J = 6.5 Hz, 3H), 1.54-1.52 (m, 2H), 1.08-1.04 (m, 2H). |
| 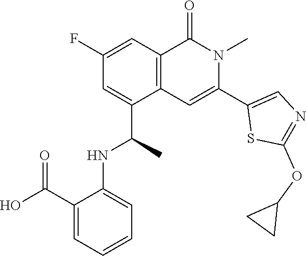 [SMILES: C[C@@H](Nc1ccccc1C(=O)O)c1cc(F)cc2c(=O)n(C)c(-c3cnc(OC4CC4)s3)cc12] C₂₅H₂₂FN₃O₄S | 345 | | ES− 477.9 [M − H] | ¹H NMR (400 MHz, DMSO-d₆) δ 12.80 (s, 1H), 8.42 (s, 1H), 7.86-7.79 (m, 2H), 7.59 (s, 1H), 7.49- 7.41 (m, 1H), 7.23- 7.13 (m, 2H), 6.59-6.51 (m, 1H), 6.31 (d, J = 8.4 Hz, 1H), 5.30 (s, 1H), 4.42- 4.33 (m, 1H), 3.50 (s, 3H), 1.54 (d, J = 6.6 Hz, 3H), 0.96-0.83 (m, 4H). |
| 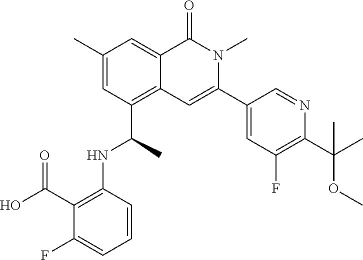 [SMILES: COC(C)(C)c1ncc(-c2cc3c([C@@H](C)Nc4cccc(F)c4C(=O)O)cc(C)cc3c(=O)n2C)cc1F] C₂₉H₂₉F₂N₃O₄ | 346 | | ES− 520.2 [M − H] | ¹H NMR (400 MHz, DMSO-d₆) δ 8.65 (d, J = 1.8 Hz, 1H), 8.27-8.19 (m, 1H), 8.08-8.05 (m, 1H), 8.00 (s, 1H), 7.52 (d, J = 1.9 Hz, 1H), 7.01 (d, J = 9.1 Hz, 2H), 6.28-6.23 (m, 1H), 6.07 (d, J = 8.4 Hz, 1H), 5.20 (d, J = 6.7 Hz, 1H), 3.38 (s, 3H), 3.06 (s, 3H), 2.38 (s, 3H), 1.61 (s, 6H), 1.47 (d, J = 6.5 Hz, 3H). |
| 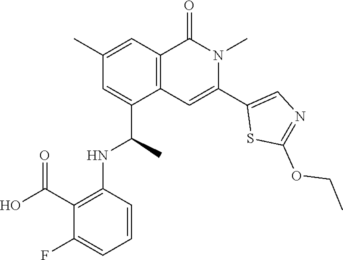 [SMILES: CCOc1ncc(-c2cc3c([C@@H](C)Nc4cccc(F)c4C(=O)O)cc(C)cc3c(=O)n2C)s1] C₂₅H₂₄FN₃O₄S | 347 | | ES− 480.1 [M − H] | ¹H NMR (400 MHz, DMSO-d₆) δ 8.22 (s, 1H), 7.97 (s, 1H), 7.50 (d, J = 3.9 Hz, 2H), 7.06 (s, 1H), 7.03 (s, 1H), 6.27 (t, J = 9.7 Hz, 1H), 6.08 (d, J = 8.4 Hz, 1H), 5.15 (s, 1H), 4.50 (q, J = 7.0 Hz, 2H), 3.48 (s, 3H), 2.37 (s, 3H), 1.48 (d, J = 6.5 Hz, 3H), 1.40 (t, J = 7.0 Hz, 3H). |
| 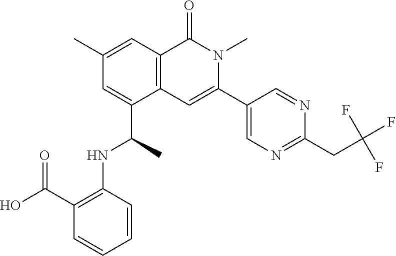 [SMILES: Cc1cc([C@@H](C)Nc2ccccc2C(=O)O)c2cc(-c3cnc(CC(F)(F)F)nc3)n(C)c(=O)c2c1] C₂₆H₂₃F₃N₄O₃ | 348 | | ES− 495.1 [M − H] | ¹H NMR (400 MHz, DMSO-d₆) δ 12.76 (s, 1H), 9.15 (s, 2H), 8.42 (s, 1H), 8.01 (s, 1H), 7.84-7.78 (m, 1.7 Hz, 1H), 7.50 (d, J = 1.9 Hz, 1H), 7.14 (d, J = 17.3 Hz, 2H), 6.54-6.50 (m, 1H), 6.34 (d, J = 8.5 Hz, 1H), 5.28 (s, 1H), 4.15- 4.07 (m, 2H), 3.39 (s, 3H), 2.38 (s, 3H), 1.51 (d, J = 6.5 Hz, 3H). |
| 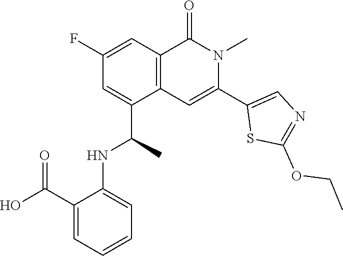 [SMILES: CCOc1ncc(-c2cc3c([C@@H](C)Nc4ccccc4C(=O)O)cc(F)cc3c(=O)n2C)s1] C₂₄H₂₂FN₃O₄S | 349 | | ES− 466.0 [M − H] | ¹H NMR (400 MHz, DMSO-d₆) δ 8.47 (s, 1H), 7.85-7.78 (m, 2H), 7.52 (s, 1H), 7.45 (d, J = 9.9, 2.8 Hz, 1H), 7.15 (d, J = 8.4 Hz, 2H), 6.57-6.51 (m, 1H), 6.30 (d, J = 8.6 Hz, 1H), 5.29 (s, 1H), 4.50 (q, J = 7.0 Hz, 2H), 3.50 (s, 3H), 1.53 (d, J = 6.5 Hz, 3H), 1.44-1.38 (m, 3H). |
| 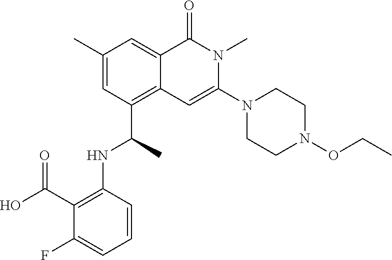 [SMILES: CCON1CCN(c2cc3c([C@@H](C)Nc4cccc(F)c4C(=O)O)cc(C)cc3c(=O)n2C)CC1] C₂₆H₃₁FN₄O₄ | 350 | | ES+ 483.1 [M + H] | ¹H NMR (400 MHz, DMSO-d₆) δ 8.35 (s, 1H), 7.85 (s, 1H), 7.44 (s, 1H), 6.92 (s, 1H), 6.39 (s, 1H), 6.19 (s, 1H), 6.01 (s, 1H), 5.03 (s, 1H), 3.75-3.66 (m, 2H), 3.49 (s, 3H), 3.19 (s, 4H), 2.92 (s, 2H), 2.67 (s, 2H), 2.32 (s, 3H), 1.47 (d, J = 6.2 Hz, 3H), 1.17-1.09 (m, 3H). |
| 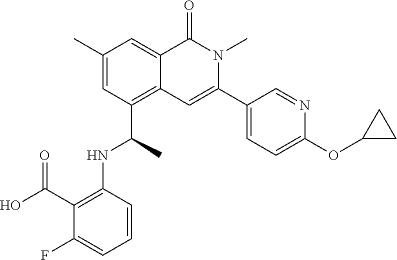 [SMILES: Cc1cc([C@@H](C)Nc2cccc(F)c2C(=O)O)c2cc(-c3ccc(OC4CC4)nc3)n(C)c(=O)c2c1] C₂₈H₂₆FN₃O₄ | 351 | | ES+ 488.4 [M + H] | ¹H NMR (400 MHz, DMSO-d₆) δ 13.20 (s, 1H), 8.44 (d, J = 2.5 Hz, 1H), 8.13 (d, J = 5.3 Hz, 1H), 8.03-7.96 (m, 2H), 7.48 (d, J = 1.9 Hz, 1H), 7.15- 7.12 (m, 1H), 7.00 (d, J = 8.6 Hz, 1H), 6.90 (s, 1H), 6.38-6.32 (m, 1H), 6.15 (d, J = 8.6 Hz, 1H), 5.26- 5.19 (m, 1H), 4.31-4.29 (m, 1H), 3.36 (s, 3H), 2.38 (s, 3H), 1.49 (d, J = 6.6 Hz, 3H), 0.86-0.75 (m, 2H), 0.75-0.73 (m, 2H). |
| 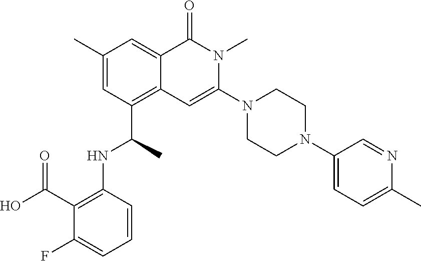 [SMILES: Cc1cc([C@@H](C)Nc2cccc(F)c2C(=O)O)c2cc(N3CCN(c4ccc(C)nc4)CC3)n(C)c(=O)c2c1] C₃₀H₃₂FN₅O₃ | 352 | | ES+ 530.3 [M + H] | ¹H NMR (400 MHz, DMSO-d₆) δ 8.23 (d, J = 2.9 Hz, 1H), 8.18 (s, 1H), 7.89 (s, 1H), 7.43 (s, 1H), 7.35-7.31 (m, 1H), 7.12 (d, J = 8.4 Hz, 2H), 6.43 (s, 1H), 6.30 (s, 1H), 6.18 (d, J = 7.8 Hz, 1H), 5.14 (s, 1H), 3.54 (s, 3H), 3.14 (s, 8H), 2.37 (s, 3H), 2.34 (s, 3H), 1.53 (d, J = 6.6 Hz, 3H). |
| 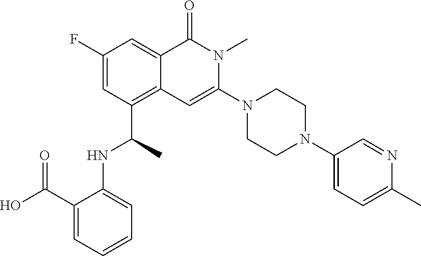 [SMILES: Cc1ccc(N2CCN(c3cc4c([C@@H](C)Nc5ccccc5C(=O)O)cc(F)cc4c(=O)n3C)CC2)cn1] C₂₉H₃₀FN₅O₃ | 353 | | ES+ 516.2 [M + H] | ¹H NMR (400 MHz, DMSO-d₆) δ 8.80 (s, 1H), 8.24-8.23 (d, J = 3.0 Hz, 1H), 7.83 (d, J = 7.7 Hz, 1H), 7.73-7.72 (m, 1H), 7.41-7.39 (m, 1H), 7.35- 7.34 (m, 1H), 7.12 (d, J = 8.5 Hz, 2H), 6.55-6.47 (m, 2H), 6.31 (d, J = 8.4 Hz, 1H), 5.24 (d, J = 6.8 Hz, 1H), 3.56 (s, 3H), 3.17 (s, 8H), 2.37 (s, 3H), 1.55 (d, J = 6.5 Hz, 3H). |
| 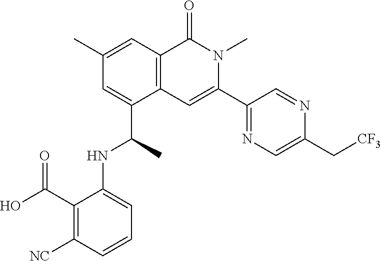 [SMILES: Cc1cc([C@@H](C)Nc2cccc(C#N)c2C(=O)O)c2cc(-c3cnc(CC(F)(F)F)cn3)n(C)c(=O)c2c1] C₂₇H₂₈F₃N₅O₃ | 354 | | ES+ 528.2 [M + H] | ¹H NMR (400 MHz, CDCl₃) 8 8.18-8.11 (m, 1H), 7.49- 7.42 (m, 1H), 7.42-7.36 (m, 1H), 7.22-7.12 (m, 1H), 6.57-6.49 (m, 1H), 6.45-6.37 (m, 1H), 6.25- 6.18 (m, 1H), 5.09-4.99 (m, 1H), 3.65 (s, 3H), 3.12- 3.10 (m, 2H), 3.06-2.75 (m, 8H), 2.41 (s, 3H), 1.72- 1.68 (m, 3H). |
| 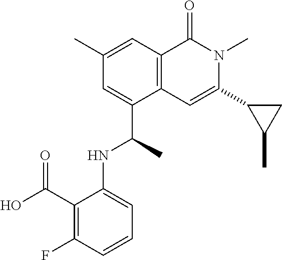 C₂₄H₂₅FN₂O₃ | 355 | trans cyclopropyl diastereomer 1 | ES+ 409.2 [M + H] | ¹H NMR (400 MHz, CDCl₃) 8 8.53-8.34 (m, 1H), 8.14 (s, 1H), 7.42 (s, 1H), 7.14- 6.99 (m, 1H), 6.50 (s, 1H), 6.36-6.21 (m, 1H), 6.11- 5.96 (m, 1H), 5.04-4.88 (m, 1H), 3.77 (s, 3H), 2.37 (s, 3H), 1.61 (d, J = 6.0 Hz, 4H), 1.30 (s, 3H), 1.06 (s, 2H), 0.83 (d, J = 8.4 Hz, 1H). |
| 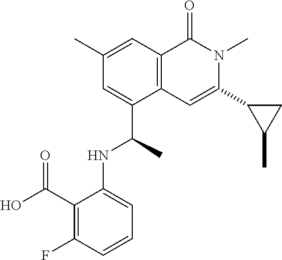 C₂₄H₂₅FN₂O₃ | 356 | trans cyclopropyl diastereomer 2 | ES+ 409.2 [M + H] | ¹H NMR (400 MHz, CDCl₃) 8 8.46 (s, 1H), 8.14 (s, 1H), 7.42 (s, 1H), 7.13- 7.02 (m, 1H), 6.49 (s, 1H), 6.31-6.25 (m, 1H), 6.04 (d, J = 8.8 Hz, 1H), 5.01- 4.95 (m, 1H), 3.78 (s, 3H), 2.39 (s, 3H), 1.67-1.58 (m, 4H), 1.32 (d, J = 6.0 Hz, 3H), 1.15-1.10 (m, 1H), 1.08-1.01 (m, 1H), 0.87-0.80 (m, 1H). |
| 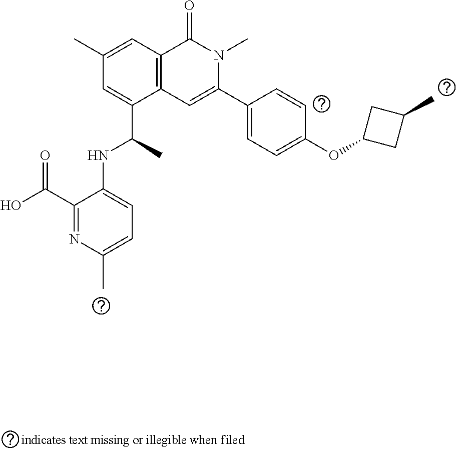 C₂₈H₂₆ClFN₄O₄ | 357 | | ES+ 537.4 [M + H] | ¹H NMR (400 MHz, DMSO-d₆) δ 10.05 (d, J = 6.5 Hz, 1H), 8.40 (d, J = 2.5 Hz, 1H), 8.03-7.94 (m, 2H), 7.49 (d, J = 1.9 Hz, 1H), 6.96 (d, J = 8.6 Hz, 1H), 6.93-6.85 (m, 2H), 6.53 (d, J = 8.6 Hz, 1H), 5.50-5.25 (m, 2H), 5.10-5.07 (m, 1H), 3.34 (s, 3H), 2.78-2.58 (m, 2H), 2.58-2.51 (m, 2H), 2.36 (s, 3H), 1.45 (d, J = 6.5 Hz, 3H). |
| 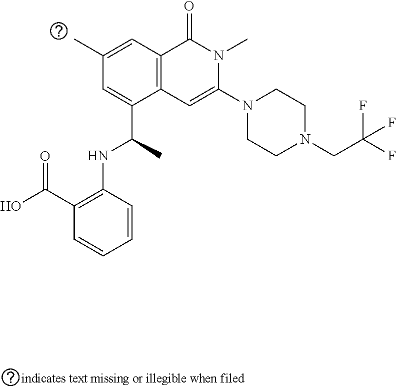 [SMILES: Cc1cc([C@@H](C)Nc2ccccc2C(=O)O)c2cc(N3CCN(CC(F)(F)F)CC3)n(C)c(=O)c2c1] C₂₅H₂₆ClF₃N₄O₃ | 358 | | ES− 521.0 [M − H] | ¹H NMR (400 MHz, Chloroform-d) δ 8.27 (d, J = 2.3 Hz, 1H), 8.15 (s, 1H), 8.04-7.98 (m, 1H), 7.60 (d, J = 2.3 Hz, 1H), 7.25-7.17 (m, 1H), 6.66-6.58 (m, 1H), 6.27-6.20 (m, 2H), 4.93 (s, 1H), 3.62 (s, 3H), 3.08 (q, J = 9.5 Hz, 5H), 2.82 (s, 5H), 1.65 (d, J = 6.7 Hz, 3H). |
| 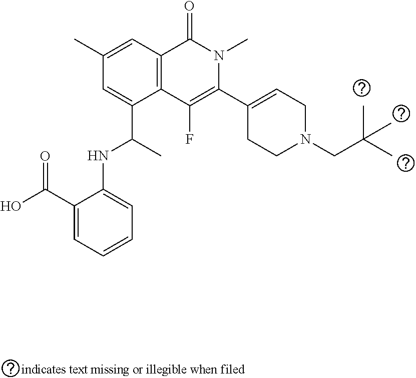 [SMILES: Cc1cc(C(C)Nc2ccccc2C(=O)O)c2c(F)c(C3=CCN(CC(C)(C)C)CC3)n(C)c(=O)c2c1] C₂₇H₂₇F₄N₃O₃ | 359 | enantiomer 1 | ES− 516.2 (M − H) | ¹H NMR (400 MHz, DMSO-d₆) δ 8.80 (s, 1H), 8.04 (d, J = 2.5 Hz, 1H), 7.86-7.79 (m, 1H), 7.66- 7.61 (m, 1H), 7.15- 7.07 (m, 1H), 6.54-6.45 (m, 1H), 6.23 (d, J = 8.5 Hz, 1H), 6.06 (s, 1H), 5.29 (d, J = 6.8 Hz, 1H), 3.40 (s, 5H), 3.36 (d, J = 10.1 Hz, 2H), 2.94 (s, 2H), 2.36 (d, J = 7.8 Hz, 5H), 1.51 (d, J = 6.5 Hz, 3H). |
| 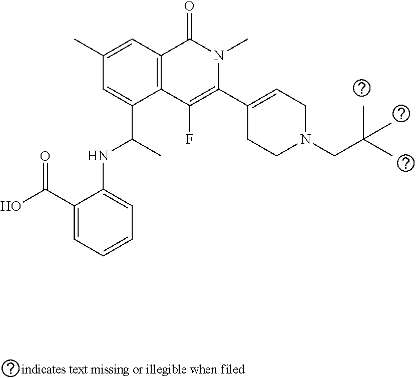 [SMILES: Cc1cc(C(C)Nc2ccccc2C(=O)O)c2c(F)c(C3=CCN(CC(C)(C)C)CC3)n(C)c(=O)c2c1] C₂₇H₂₇F₄N₃O₃ | 360 | enantiomer 2 | ES− 516.2 [M − H] | ¹H NMR (400 MHz, DMSO-d₆) δ 8.53 (s, 1H), 8.04 (s, 1H), 7.86-7.79 (m, 1H), 7.62 (s, 1H), 7.21- 7.12 (m, 1H), 6.57- 6.49 (m, 1H), 6.27 (d, J = 8.5 Hz, 1H), 6.06 (s, 1H), 5.31 (d, J = 7.0 Hz, 1H), 3.40 (s, 5H), 3.36 (d, J = 10.1 Hz, 2H), 2.95 (d, J = 5.6 Hz, 2H), 2.36 (d, J = 9.0 Hz, 5H), 1.53 (d, J = 6.5 Hz, 3H). |
| 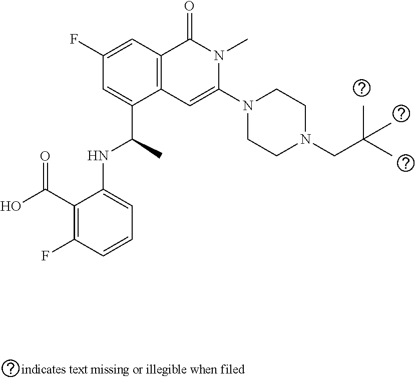 C₂₅H₂₅F₅N₄O₃ | 361 | | ES+ 525.2 [M + H] | ¹H NMR (400 MHz, DMSO-d₆) δ 8.27 (s, 1H), 7.72-7.65 (m, 1H), 7.44- 7.36 (m, 1H), 6.96 (s, 1H), 6.45 (s, 1H), 6.29-6.20 (m, 1H), 6.03 (d, J = 8.1 Hz, 1H), 5.16 (d, J = 6.8 Hz, 1H), 3.51 (s, 3H), 3.30- 3.24 (m, 2H), 3.09-2.66 (m, 8H), 1.49 (d, J = 6.5 Hz, 3H). |
| 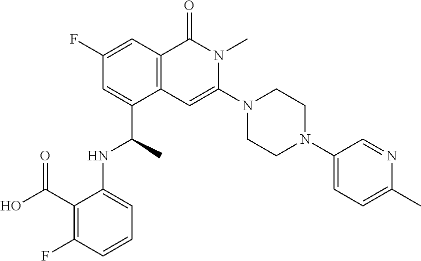 [SMILES: Cc1ccc(N2CCN(c3cc4c([C@@H](C)Nc5cccc(F)c5C(=O)O)cc(F)cc4c(=O)n3C)CC2)cn1] C₂₉H₂₉F₂N₅O₃ | 362 | | ES+ 534.3 [M + H] | ¹H NMR (400 MHz, DMSO-d₆) δ 8.24 (d, J = 3.0 Hz, 1H), 8.11 (d, J = 14.2 Hz, 1H), 7.75-7.72 (m, 1H), 7.42 (d, J = 2.9 Hz, 1H), 7.35-7.32 (m, 1H), 7.12 (d, J = 8.6 Hz, 2H), 6.48 (s, 1H), 6.34 (d, J = 9.3 Hz, 1H), 6.16 (s, 1H), 5.24 (s, 1H), 3.56 (s, 3H), 3.28-2.85 (m, 8H), 2.38 (s, 3H), 1.56 (d, J = 6.6 Hz, 3H). |
| 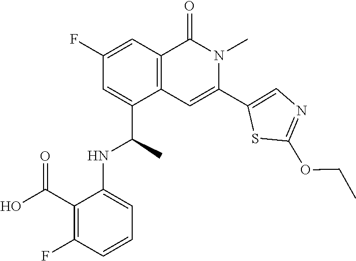 [SMILES: CCOc1ncc(-c2cc3c([C@@H](C)Nc4cccc(F)c4C(=O)O)cc(F)cc3c(=O)n2C)s1] C₂₄H₂₁F₂N₃O₄S | 363 | | ES− 484.1 [M − H] | ¹H NMR (400 MHz, DMSO-d₆) δ 8.08 (s, 1H), 7.82 (d, J = 8.9 Hz, 1H), 7.49 (d, J = 22.5 Hz, 2H), 7.13 (s, 2H), 6.40-6.31 (m, 1H), 6.10 (d, J = 8.6 Hz, 1H), 5.27 (s, 1H), 4.55- 4.46 (m, 2H), 3.50 (s, 3H), 1.52 (d, J = 6.5 Hz, 3H), 1.45-1.37 (m, 3H). |
| 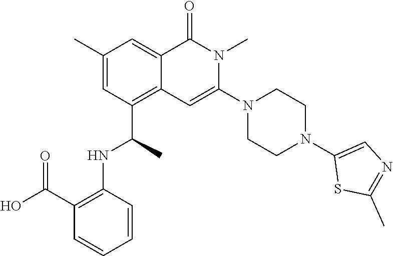 [SMILES: Cc1cc([C@@H](C)Nc2ccccc2C(=O)O)c2cc(N3CCN(c4cnc(C)s4)CC3)n(C)c(=O)c2c1] C₂₈H₃₁N₅O₃S | 364 | | ES+ 518.1 [M + H] | ¹H NMR (400 MHz, Chloroform-d) δ 8.27 (s, 1H), 8.13 (s, 1H), 8.05- 7.97 (m, 1H), 7.50 (s, 1H), 7.25-7.17 (m, 1H), 6.94 (s, 1H), 6.65-6.55 (m, 1H), 6.41 (s, 1H), 6.36 (d, J = 8.5 Hz, 1H), 5.02-4.92 (m, 1H), 3.65 (s, 3H), 3.50- 2.95 (m, 8H), 2.66 (s, 3H), 2.42 (s, 3H), 1.70 (d, J = 6.7 Hz, 3H). |
| 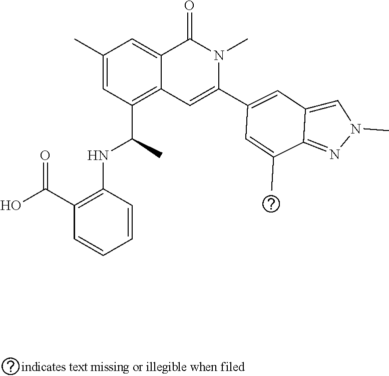 [SMILES: Cc1cc([C@@H](C)Nc2ccccc2C(=O)O)c2cc(-c3cc(C)c4nn(C)cc4c3)n(C)c(=O)c2c1] C₂₈H₂₅FN₄O₃ | 365 | | ES− 483.3 [M − H] | ¹H NMR (400 MHz, DMSO-d₆) δ 12.73 (s, 1H), 8.60 (d, J = 2.7 Hz, 1H), 8.39 (d, J = 5.8 Hz, 1H), 8.00 (s, 1H), 7.84-7.78 (m, 2H), 7.59-7.49 (m, 1H), 7.36-7.28 (m, 1H), 7.22-7.13 (m, 1H), 6.91 (s, 1H), 6.57-6.48 (m, 1H), 6.35 (d, J = 8.5 Hz, 1H), 5.25-5.18 (m, 1H), 4.25 (s, 3H), 3.37 (s, 3H), 2.38 (s, 3H), 1.52 (d, J = 6.5 Hz, 3H). |
| 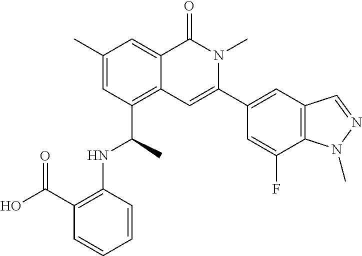 [SMILES: Cc1cc([C@@H](C)Nc2ccccc2C(=O)O)c2cc(-c3cc(F)c4c(cnn4C)c3)n(C)c(=O)c2c1] C₂₈H₂₅FN₄O₃ | 366 | | ES− 483.3 [M − H] | ¹H NMR (400 MHz, DMSO-d₆) δ 12.73 (s, 1H), 8.38 (d, J = 5.9 Hz, 1H), 8.24 (d, J = 2.3 Hz, 1H), 8.01 (s, 1H), 7.85-7.78 (m, 2H), 7.59-7.50 (m, 1H), 7.33-7.30 (m, 1H), 7.19-7.15 (m, 1H), 6.90 (s, 1H), 6.56-6.48 (m, 1H), 6.35 (d, J = 8.4 Hz, 1H), 5.25-5.17 (m, 1H), 4.24 (s, 3H), 3.36 (s, 3H), 2.38 (s, 3H), 1.52 (d, J = 6.5 Hz, 3H). |
| 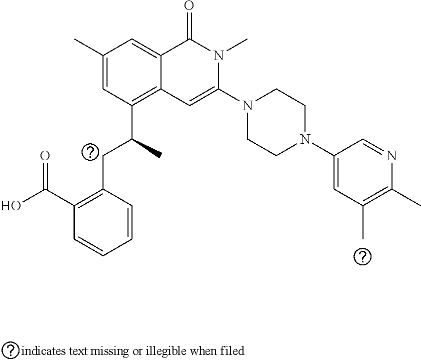 C₃₀H₃₂FN₅O₃ | 367 | | ES+ 530.4 [M + H] | ¹H NMR (400 MHz, DMSO-d₆) δ 12.74 (s, 1H), 8.38 (d, J = 6.0 Hz, 1H), 8.14 (s, 1H), 7.90 (s, 1H), 7.80 (d, J = 7.9 Hz, 1H), 7.44 (s, 1H), 7.36 (d, J = 12.9 Hz, 1H), 7.25-7.12 (m, 1H), 6.56-6.50 (m, 1H), 6.47-6.37 (m, 2H), 5.18-5.17 (m, 1H), 3.92- 3.64 (m, 2H), 3.55 (s, 3H), 3.25-2.98 (m, 6H), 2.39-2.18 (m, 6H), 1.56 (d, J = 6.5 Hz, 3H). |
| 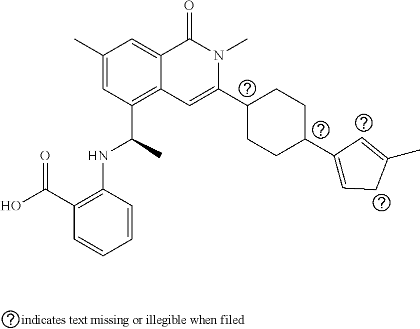 [SMILES: CC1=CC(C2CCC(c3cc4c([C@@H](C)Nc5ccccc5C(=O)O)cc(C)cc4c(=O)n3C)CC2)=CC1] C₂₈H₃₁N₅O₃S | 368 | | ES+ 540.5 [M+23] | ¹H NMR (400 MHz, DMSO-d₆) δ 8.49 (s, 1H), 7.88 (s, 1H), 7.79 (d, J = 1.7 Hz, 1H), 7.44 (s, 1H), 7.20-7.11 (m, 1H), 6.50- 6.45 (m, 1H), 6.41- 6.38 (m, 2H), 6.13 (s, 1H), 5.15-5.11 (m, 1H), 4.00- 3.60 (m, 2H), 3.53 (s, 3H), 3.20-2.80 (m, 6H), 2.58 (s, 3H), 2.33 (s, 3H), 1.54 (d, J = 6.6 Hz, 3H). |
| 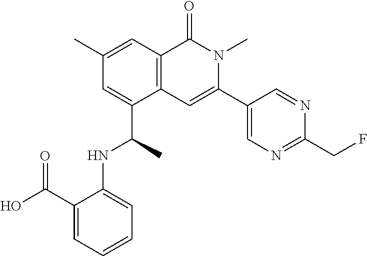 [SMILES: Cc1cc([C@@H](C)Nc2ccccc2C(=O)O)c2cc(-c3cnc(CF)nc3)n(C)c(=O)c2c1] C₂₅H₂₃FN₄O₃ | 369 | | ES− 445.2 [M − H] | ¹H NMR (400 MHz, DMSO-d₆) δ 12.76 (s, 1H), 9.16 (s, 2H), 8.42 (s, 1H), 8.01 (s, 1H), 7.83-7.78 (m, 1H), 7.51 (d, J = 1.9 Hz, 1H), 7.20-7.12 (m, 1H), 7.09 (s, 1H), 6.55- 6.48 (m, 1H), 6.35 (d, J = 8.5 Hz, 1H), 5.70 (s, 1H), 5.58 (s, 1H), 5.31-5.21 (m, 1H), 3.40 (s, 3H), 2.38 (s, 3H), 1.51 (d, J = 6.5 Hz, 3H). |
| 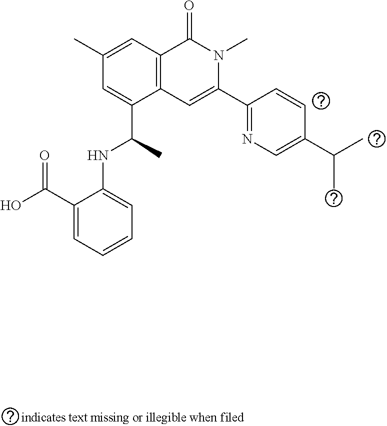 C₂₅H₂₂F₂N₄O₃ | 370 | | ES+ 465.2 [M + H] | ¹H NMR (400 MHz, DMSO-d₆) δ 12.84 (s, 1H), 9.20 (d, J = 1.5 Hz, 1H), 9.12 (s, 1H), 8.48 (s, 1H), 8.04 (s, 1H), 7.82-7.80 (m, 1H), 7.51 (d, J = 1.9 Hz, 1H), 7.39-7.25 (m, 2H), 7.18-7.14 (m, 1H), 6.50 (d, J = 7.5 Hz, 1H), 6.36 (d, J = 8.5 Hz, 1H), 5.33-5.30 (m, 1H), 3.47 (s, 3H), 2.40 (s, 3H), 1.52 (d, J = 6.4 Hz, 3H). |
| 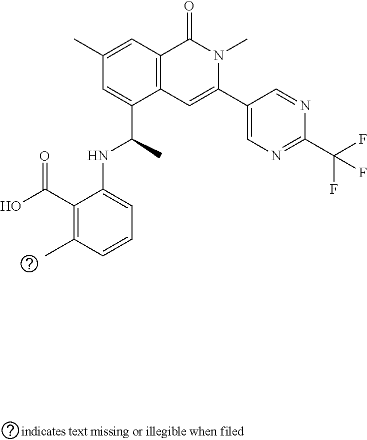 C₂₅H₂₀F₄N₄O₃ | 371 | | ES+ 501.0 [M + H] | ¹H NMR (400 MHz, DMSO-d₆) δ 13.22 (s, 1H), 9.39 (s, 2H), 8.16 (d, J = 6.0 Hz, 1H), 8.04 (s, 1H), 7.51 (d, J = 1.9 Hz, 1H), 7.19 (s, 1H), 7.20-7.10 (m, 1H), 6.39-6.30 (m, 1H), 6.15 (d, J = 8.6 Hz, 1H), 5.28-5.20 (m, 1H), 3.44 (s, 3H), 2.41 (s, 3H), 1.51 (d, J = 6.5 Hz, 3H). |
| 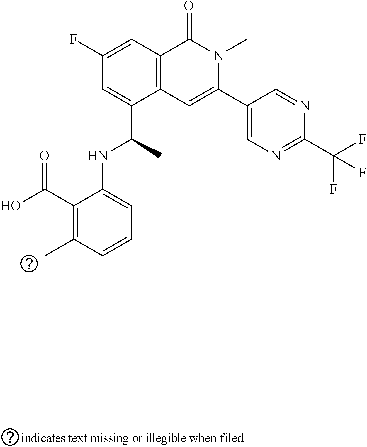 [SMILES: Cc1cccc(N[C@H](C)c2cc(F)cc3c(=O)n(C)c(-c4cnc(C(F)(F)F)nc4)cc23)c1C(=O)O] C₂₄H₁₇F₅N₄O₃ | 372 | | ES− 503.2 [M − H] | ¹H NMR (400 MHz, DMSO-d₆) δ 13.26 (s, 1H), 9.40 (s, 2H), 8.08 (d, J = 6.0 Hz, 1H), 7.91-7.84 (m, 1H), 7.52-7.46 (m, 1H), 7.24 (s, 1H), 7.18- 7.09 (m, 1H), 6.34-6.31 (m, 1H), 6.10 (d, J = 8.5 Hz, 1H), 5.36-5.22 (m, 1H), 3.45 (s, 3H), 1.53 (d, J = 6.6 Hz, 3H). |
| 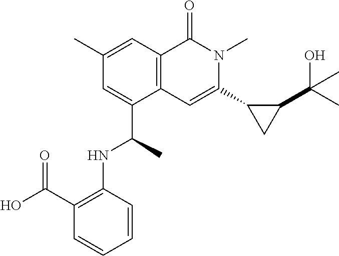 [SMILES: Cc1cc([C@@H](C)Nc2ccccc2C(=O)O)c2cc([C@H]3C[C@@H]3C(C)(C)O)n(C)c(=O)c2c1] C₂₆H₃₀N₂O₄ | 373 | trans cyclopropyl diastereomer 1 | ES+ 457.2 [M + Na] | ¹H NMR (400 MHz, Methanol-d₄) δ 8.01 (s, 1H), 7.91-7.81 (m, 1H), 7.58 (d, J = 1.2 Hz, 1H), 7.18-7.09 (m, 1H), 6.75 (s, 1H), 6.58-6.47 (m, 1H), 6.39 (d, J = 8.4 Hz, 1H), 5.14-5.07 (m, 1H), 3.82 (s, 3H), 2.39 (s, 3H), 2.14-2.01 (m, 1H), 1.62 (d, J = 6.4 Hz, 3H), 1.45- 1.39 (m, 1H), 1.33 (s, 3H), 1.25 (s, 3H), 1.17-1.09 (m, 1H), 0.83-0.78 (m, 1H). |
| 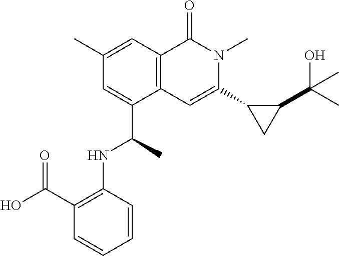 [SMILES: Cc1cc([C@@H](C)Nc2ccccc2C(=O)O)c2cc([C@H]3C[C@@H]3C(C)(C)O)n(C)c(=O)c2c1] C₂₆H₃₀N₂O₄ | 374 | trans cyclopropyl diastereomer 2 | ES+ 457.2 [M + Na] | ¹H NMR (400 MHz, Methanol-d₄) δ 7.92 (s, 1H), 7.79 (d, J = 7.2 Hz, 1H), 7.47 (d, J = 1.2 Hz, 1H), 7.05 (t, J = 7.6 Hz, 1H), 6.70 (s, 1H), 6.43 (t, J = 7.2 Hz, 1H), 6.32 (d, J = 8.4 Hz, 1H), 5.03-4.97 (m, 1H), 3.72 (s, 3H), 2.30 (s, 3H), 2.02-1.95 (m, 1H), 1.53 (d, J = 6.4 Hz, 3H), 1.43-1.37 (m, 1H), 1.23 (s, 3H), 1.17 (s, 3H), 1.05-0.99 (m, 1H), 0.73- 0.67 (m, 1H). |
| 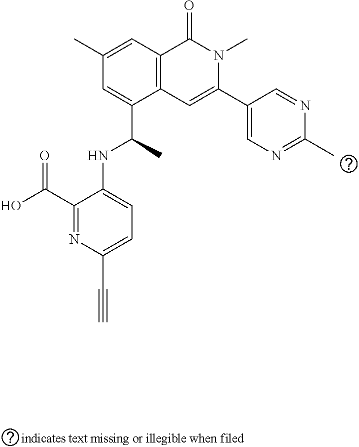 C₂₆H₂₀F₃N₅O₃ | 375 | | ES+ 508.1 [M + H] | ¹H NMR (400 MHz, CDCl₃) δ 9.08 (s, 2H), 8.48 (d, J = 5.2 Hz, 1H), 8.25 (s, 1H), 7.52 (d, J = 1.6 Hz, 1H), 7.29 (d, J = 8.8 Hz, 1H), 6.66 (s, 1H), 6.59 (d, J = 8.8 Hz, 1H), 5.06-4.92 (m, 1H), 3.50 (s, 3H), 3.06 (s, 1H), 2.46 (s, 3H), 1.69 (d, J = 6.8 Hz, 3H). |
| 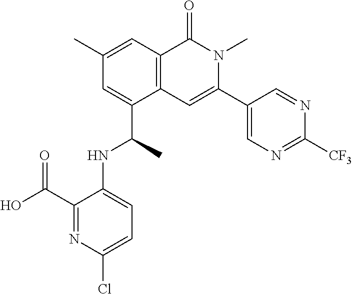 [SMILES: Cc1cc([C@@H](C)Nc2ccc(Cl)nc2C(=O)O)c2cc(-c3cnc(C(F)(F)F)nc3)n(C)c(=O)c2c1] C₂₄H₁₉ClF₃N₅O₃ | 376 | | ES+ 518.1 [M + H] | ¹H NMR (400 MHz, Methanol-d₄) δ 9.34- 9.20 (m, 2H), 8.22-8.07 (m, 1H), 7.61 (d, J = 1.2 Hz, 1H), 7.18 (d, J = 8.8 Hz, 1H), 7.13 (s, 1H), 6.95- 6.80 (m, 1H), 5.29-5.22 (m, 1H), 3.51 (s, 3H), 2.45 (s, 3H), 1.64 (d, J = 6.8 Hz, 3H). |
| 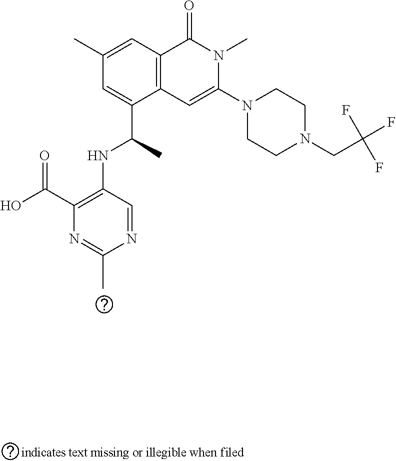 C₂₄H₂₆ClF₃N₆O₃ | 377 | | ES+ 539.3 [M + H] | ¹H NMR (400 MHz, CDCl₃) δ 8.14 (s, 1H), 7.95 (d, J = 5.2 Hz, 1H), 7.91 (s, 1H), 7.37 (d, J = 1.2 Hz, 1H), 6.17 (s, 1H), 5.08-4.95 (m, 1H), 3.63 (s, 3H), 3.17- 3.10 (m, 2H), 3.09-2.75 (m, 8H), 2.39 (s, 3H), 1.71 (d, J = 6.4 Hz, 3H). |
| 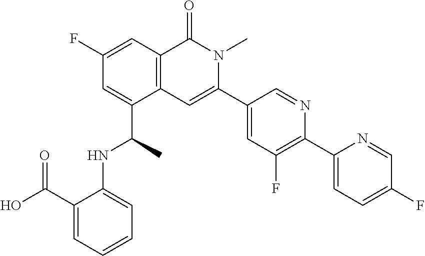 [SMILES: C[C@@H](Nc1ccccc1C(=O)O)c1cc(F)cc2c(=O)n(C)c(-c3cnc(-c4ccc(F)cn4)c(F)c3)cc12] C₂₉H₂₁F₃N₄O₃ | 378 | | ES+ 531.2 [M + H] | ¹H NMR (400 MHz, DMSO-d₆) δ 12.86 (s, 1H), 8.84 (s, 1H), 8.77 (d, J = 3.0 Hz, 1H), 8.71-8.31 (m, 1H), 8.31-8.23 (m, 1H), 8.23-8.15 (m, 1H), 8.01-7.91 (m, 1H), 7.90- 7.79 (m, 2H), 7.50- 7.42 (m, 1H), 7.15 (d, J = 7.3 Hz, 2H), 6.57-6.48 (m, 1H), 6.30 (d, J = 8.5 Hz, 1H), 5.34 (s, 1H), 3.45 (s, 3H), 1.54 (d, J = 6.5 Hz, 3H). |
| 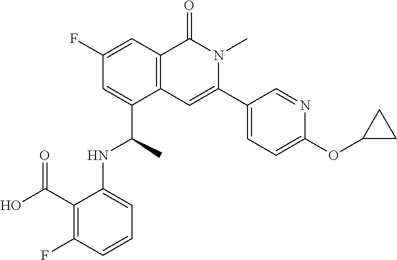 [SMILES: C[C@@H](Nc1cccc(F)c1C(=O)O)c1cc(F)cc2c(=O)n(C)c(-c3ccc(OC4CC4)nc3)cc12] C₂₇H₂₃F₂N₃O₄ | 379 | | ES− 490.2 [M − H] | ¹H NMR (400 MHz, DMSO-d₆) δ 8.46 (d, J = 2.5 Hz, 1H), 8.25 (s, 1H), 8.05-7.89 (m, 1H), 7.85- 7.77 (m, 1H), 7.51-7.44 (m, 1H), 7.01 (d, J = 8.6 Hz, 1H), 6.96 (s, 1H), 6.95 (d, J = 14.7 Hz, 1H), 6.30- 6.21 (m, 1H), 6.02 (d, J = 8.4 Hz, 1H), 5.23 (d, J = 6.9 Hz, 1H), 4.33-4.24 (m, 1H), 3.37 (s, 3H), 1.48 (d, J = 6.6 Hz, 3H), 0.88-0.76 (m, 2H), 0.79-0.68 (m, 2H). |
| 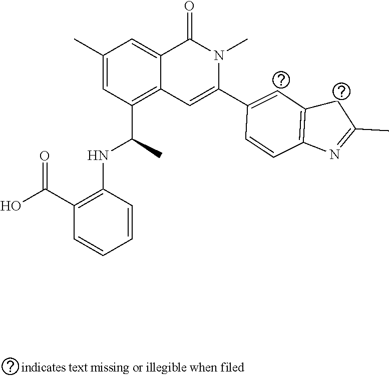 [SMILES: CC1=Nc2ccc(-c3cc4c([C@@H](C)Nc5ccccc5C(=O)O)cc(C)cc4c(=O)n3C)cc2C1] C₂₇H₂₄N₄O₃S | 380 | | ES− 483.4 [M − H] | ¹H NMR (400 MHz, DMSO-d₆) δ 12.86 (s, 1H), 8.58 (s, 1H), 8.48 (d, J = 8.3 Hz, 1H), 8.03 (s, 1H), 7.92 (d, J = 8.4 Hz, 1H), 7.81 (d, J = 7.9 Hz, 1H), 7.53 (s, 1H), 7.13 (d, J = 8.2 Hz, 2H), 6.54-6.46 (m, 1H), 6.33 (d, J = 8.5 Hz, 1H), 5.24-5.15 (m, 1H), 3.45 (s, 3H), 2.90 (s, 3H), 2.39 (s, 3H), 1.52 (d, J = 6.5 Hz, 3H). |
| 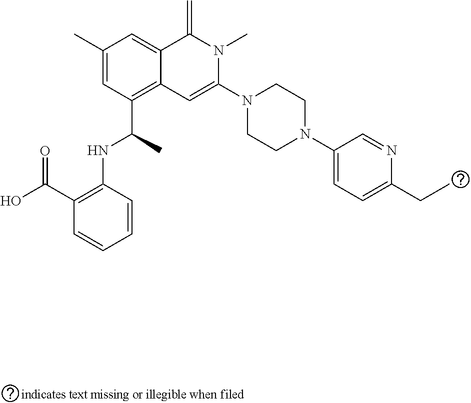 [SMILES: C=C1c2cc(C)cc([C@@H](C)Nc3ccccc3C(=O)O)c2C=C(N2CCN(c3ccc(CC)nc3)CC2)N1C] C₃₀H₃₂FN₅O₃ | 381 | | ES+ 530.3 [M + H] | ¹H NMR (400 MHz, Methanol-d₄) δ 8.33 (d, J = 2.8 Hz, 1H), 8.03-7.98 (m, 1H), 7.94-7.87 (m, 1H), 7.57 (d, J = 1.9 Hz, 1H), 7.55-7.50 (m, 1H), 7.46 (d, J = 8.7 Hz, 1H), 7.20-7.11 (m, 1H), 6.60 (s, 1H), 6.58-6.49 (m, 1H), 6.41 (d, J = 8.4 Hz, 1H), 5.42 (s, 1H), 5.30 (s, 1H), 5.14-5.10 (m, 1H), 4.00-3.87 (m, 2H), 3.70 (s, 3H), 3.46-3.36 (m, 3H), 3.26-2.95 (m, 3H) 2.41 (s, 3H), 1.67 (d, J = 6.7 Hz, 3H). |
| 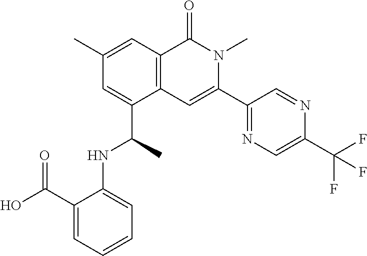 [SMILES: Cc1cc([C@@H](C)Nc2ccccc2C(=O)O)c2cc(-c3cnc(C(F)(F)F)cn3)n(C)c(=O)c2c1] C₂₅H₂₁F₃N₄O₃ | 382 | | ES+ 483.0 [M + H] | ¹H NMR (400 MHz, DMSO-d₆) δ 12.78 (s, 1H), 9.36-9.29 (m, 2H), 8.42 (d, J = 6.0 Hz, 1H), 8.05 (d, J = 1.8 Hz, 1H), 7.83- 7.79 (m, 1H), 7.54 (d, J = 1.9 Hz, 1H), 7.38 (s, 1H), 7.20-7.14 (m, 1H), 6.57- 6.49 (m, 1H), 6.36 (d, J = 8.4 Hz, 1H), 5.33-5.30 (m, 1H), 3.33 (s, 3H), 2.41 (s, 3H), 1.54 (d, J = 6.5 Hz, 3H). |
| 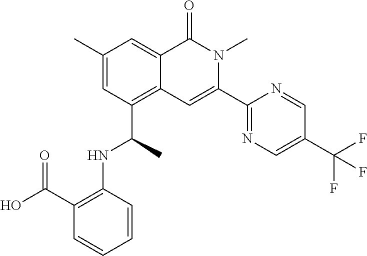 [SMILES: Cc1cc([C@@H](C)Nc2ccccc2C(=O)O)c2cc(-c3ncc(C(F)(F)F)cn3)n(C)c(=O)c2c1] C₂₅H₂₁F₃N₄O₃ | 383 | | ES− 481.1 [M − H] | ¹H NMR (400 MHz, DMSO-d₆) δ 12.80 (s, 1H), 9.50 (s, 2H), 8.56 (s, 1H), 8.05 (s, 1H), 7.78 (d, J = 7.9 Hz, 1H), 7.55 (d, J = 1.8 Hz, 1H), 7.48 (s, 1H), 7.17-7.09 (m, 1H), 6.53- 6.49 (m, 1H), 6.25 (d, J = 8.4 Hz, 1H), 5.21-5.16 (m, 1H), 3.57 (s, 3H), 2.40 (s, 3H), 1.53 (d, J = 6.5 Hz, 3H). |
| 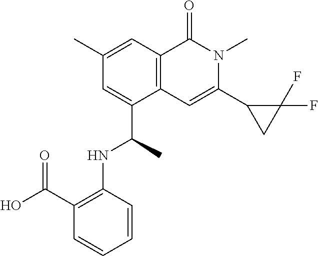 [SMILES: Cc1cc([C@@H](C)Nc2ccccc2C(=O)O)c2cc(C3CC3(F)F)n(C)c(=O)c2c1] C₂₃H₂₂F₂N₂O₃ | 384 | diastereomer 1 | ES+ 413.2 [M + H] | ¹H NMR (400 MHz, CDCl₃) δ 8.23-8.12 (m, 2H), 8.03- 7.96 (m, 1H), 7.52 (s, 1H), 7.23-7.14 (m, 1H), 6.78-6.68 (m, 1H), 6.63- 6.55 (m, 1H), 6.28 (d, J = 8.8 Hz, 1H), 5.13-4.91 (m, 1H), 3.72 (s, 3H), 2.87- 2.66 (m, 1H), 2.41 (s, 3H), 2.07-1.97 (m, 1H), 1.86-1.78 (m, 1H), 1.65 (d, J = 6.8 Hz, 3H). |
| 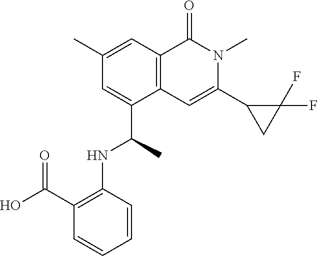 [SMILES: Cc1cc([C@@H](C)Nc2ccccc2C(=O)O)c2cc(C3CC3(F)F)n(C)c(=O)c2c1] C₂₃H₂₂F₂N₂O₃ | 385 | diastereomer 2 | ES+ 413.1 [M + H] | ¹H NMR (400 MHz, CDCl₃) δ 8.16 (s, 2H), 8.00 (d, J = 7.2 Hz, 1H), 7.52 (s, 1H), 7.26-7.16 (m, 1H), 6.71 (s, 1H), 6.61 (t, J = 7.6 Hz, 1H), 6.28 (d, J = 8.8 Hz, 1H), 5.09-4.91 (m, 1H), 3.72 (s, 3H), 2.90-2.69 (m, 1H), 2.41 (s, 3H), 2.04- 1.96 (m, 1H), 1.82-1.74 (m, 1H), 1.65 (d, J = 6.8 Hz, 3H). |
| 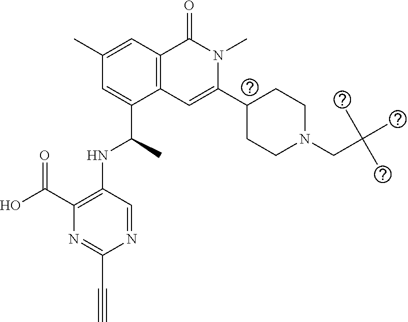 C₂₆H₂₇F₃N₆O₃ | 386 | | ES+ 529.3 [M + H] | ¹H NMR (400 MHz, CDCl₃) δ 8.21-8.11 (m, 2H), 8.00 (s, 1H), 7.38 (d, J = 1.2 Hz, 1H), 6.18 (s, 1H), 5.07- 5.03 (m, 1H), 3.63 (s, 3H), 3.23-3.12 (m, 2H), 3.12- 2.70 (m, 9H), 2.39 (s, 3H), 1.72 (d, J = 6.8 Hz, 3H). |
| 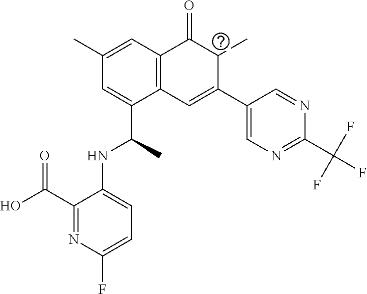 [SMILES: Cc1cc2c(c([C@@H](C)Nc3ccc(F)nc3C(=O)O)c1)C=C(c1cnc(C(F)(F)F)nc1)C(C)C2=O] C₂₄H₁₉F₄N₅O₃ | 387 | | ES+ 502.1 [M + H] | ¹H NMR (400 MHz, CDCl₃) δ 9.07 (s, 2H), 8.31-8.16 (m, 2H), 7.59-7.50 (m, 1H), 6.90-6.83 (m, 1H), 6.82-6.74 (m, 1H), 6.68 (s, 1H), 5.04-4.85 (m, 1H), 3.50 (s, 3H), 2.46 (s, 3H), 1.68 (d, J = 6.8 Hz, 3H). |
| 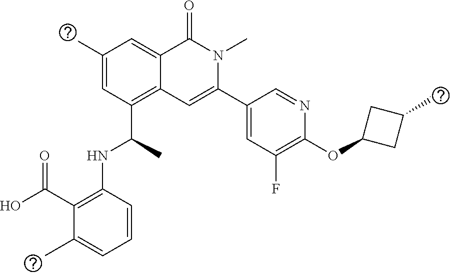 [SMILES: Cc1cc([C@@H](C)Nc2cccc(C)c2C(=O)O)c2cc(-c3cnc(O[C@H]4C[C@H](C)C4)c(F)c3)n(C)c(=O)c2c1] C₂₈H₂₃F₄N₃O₄ | 388 | | ES− 540.1 [M − H] | ¹H NMR (400 MHz, DMSO-d₆) δ 8.56 (d, J = 6.8 Hz, 1H), 8.26 (d, J = 1.9 Hz, 1H), 8.12 (d, J = 11.2 Hz, 1H), 7.80-7.77 (m, 1H), 7.55-7.51 (m, 1H), 6.99 (s, 1H), 6.75- 6.69 (m, 1H), 6.16-6.07 (m, 1H), 5.89 (d, J = 8.2 Hz, 1H), 5.52 (s, 1H), 5.31 (s, 1H), 5.15-5.12 (m, 1H), 3.37 (s, 3H), 2.84-2.50 (m, 4H), 1.43 (d, J = 6.5 Hz, 3H). |
| 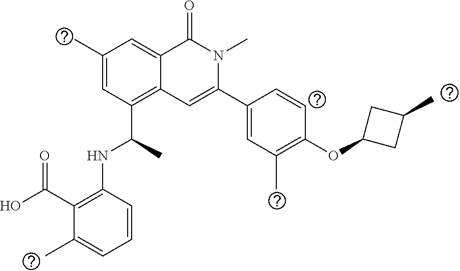 [SMILES: Cc1cc([C@@H](C)Nc2cccc(C)c2C(=O)O)c2cc(-c3ccc(O[C@H]4C[C@@H](C)C4)c(C)c3)n(C)c(=O)c2c1] C₂₈H₂₃F₄N₃O₄ | 389 | | ES+ 542.2 [M + H] | ¹H NMR (400 MHz, DMSO-d₆) δ 13.25 (s, 1H), 8.24 (d, J = 2.0 Hz, 1H), 8.15-8.08 (m, 1H), 8.06 (d, J = 5.9 Hz, 1H), 7.88-7.80 (m, 1H), 7.49-7.42 (m, 1H), 7.18-7.08 (m, 1H), 7.01 (s, 1H), 6.40-6.31 (m, 1H), 6.10 (d, J = 8.6 Hz, 1H), 5.34-5.26 (m, 1H), 5.04 (q, J = 6.5 Hz, 2H), 4.96-4.85 (m, 3H), 3.12- 3.01 (m, 2H), 2.46- 2.30 (m, 2H), 1.51 (d, J = 6.5 Hz, 3H). |
| 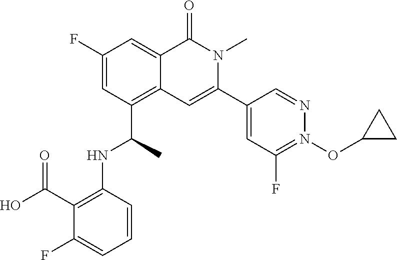 C₂₇H₂₂F₃N₃O₄ | 390 | | ES+ 510.1 [M + H] | ¹H NMR (400 MHz, DMSO-d₆) δ 13.24 (s, 1H), 8.30 (d, J = 2.0 Hz, 1H), 8.11-8.05 (m, 2.1 Hz, 2H), 7.87-7.81 (m, 2.8 Hz, 1H), 7.49-7.43 (m, 2.9 Hz, 1H), 7.16-7.09 (m, 6.0 Hz, 1H), 7.02 (s, 1H), 6.40-6.31 (m, 8.1 Hz, 1H), 6.10 (d, J = 8.6 Hz, 1H), 5.30 (s, 1H), 4.48-4.40 (m, 3.1 Hz, 1H), 3.39 (s, 3H), 1.52 (d, J = 6.5 Hz, 3H), 0.87-0.77 (m, 4H) |
| 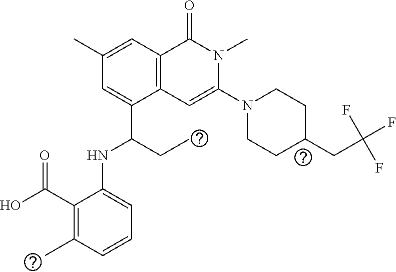 C₂₆H₂₇F₅N₄O₃ | 391 | enantiomer 1 | ES+ 539.1 [M + H] | ¹H NMR: (DMSO-d₆, 400 MHz, ppm): δ 13.28 (s, 1H), 8.46 (d, J = 6.7 Hz, 1H), 7.94 (d, J = 1.9 Hz, 1H), 7.43 (d, J = 1.9 Hz, 1H), 7.30-7.15 (m, 1H), 6.44 (s, 1H), 6.60-6.38 (m, 1H), 6.27 (d, J = 8.6 Hz, 1H), 5.55-5.35 (m, 1H), 4.87-4.60 (m, 2H), 3.49 (s, 3H), 3.39-3.20 (m, 2H), 3.27-2.82 (m, 8H), 2.33 (s, 3H). |
| 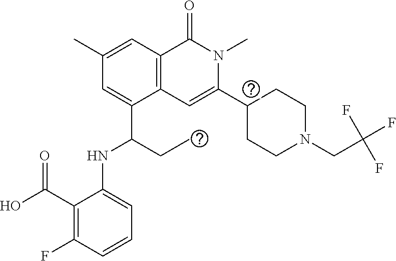 C₂₆H₂₇F₅N₄O₃ | 392 | enantiomer 2 | ES+ 539.1 [M + H] | ¹H NMR (DMSO-d₆, 400 MHz, ppm): δ 13.28 (s, 1H), 8.46 (d, J = 6.7 Hz, 1H), 7.94 (d, J = 1.9 Hz, 1H), 7.43 (d, J = 1.9 Hz, 1H), 7.30-7.15 (m, 1H), 6.44 (s, 1H), 6.60-6.38 (m, 1H), 6.27 (d, J = 8.6 Hz, 1H), 5.55-5.44 (m, 1H), 4.88-4.60 (m, 2H), 3.49 (s, 3H), 3.32-3.24 (m, 2H), 3.15-2.79 (m, 8H), 2.33 (s, 3H). |
| 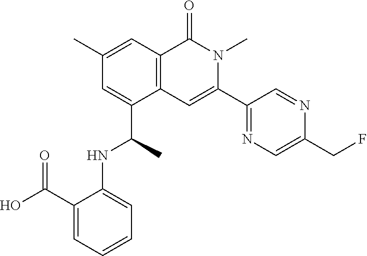 [SMILES: Cc1cc([C@@H](C)Nc2ccccc2C(=O)O)c2cc(-c3cnc(CF)cn3)n(C)c(=O)c2c1] C₂₅H₂₃FN₄O₃ | 393 | | ES− 445.3 [M − H] | ¹H NMR (400 MHz, DMSO-d₆) δ 9.10 (d, J = 1.4 Hz, 1H), 8.93 (s, 1H), 8.46 (s, 1H), 8.03 (s, 1H), 7.83-7.78 (m, 1H), 7.53 (d, J = 1.9 Hz, 1H), 7.24 (s, 1H), 7.21-7.12 (m, 1H), 6.56-6.48 (m, 1H), 6.36 (d, J = 8.5 Hz, 1H), 5.74 (s, 1H), 5.63 (s, 1H), 5.30- 5.20 (m, 1H), 3.45 (s, 3H), 2.39 (s, 3H), 1.52 (d, J = 6.5 Hz, 3H). |
| 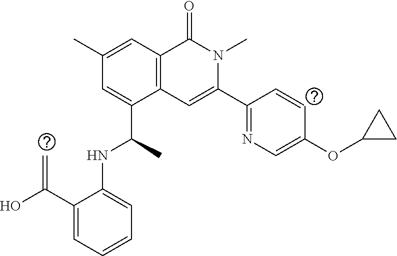 C₂₇H₂₆N₄O₄ | 394 | | ES+ 471.0 [M + H] | ¹H NMR (400 MHz, DMSO-d₆) δ 12.75 (s, 1H), 8.66 (d, J = 1.4 Hz, 1H), 8.48 (d, J = 1.3 Hz, 1H), 8.43 (d, J = 5.6 Hz, 1H), 8.02 (s, 1H), 7.83-7.79 (m, 1H), 7.51 (d, J = 1.9 Hz, 1H), 7.18-7.14 (m, 2H), 6.57-6.48 (m, 1H), 6.36 (d, J = 8.5 Hz, 1H), 5.30-5.27 (m, 1H), 4.39- 4.36 (m, 1H), 3.43 (s, 3H), 2.39 (s, 3H), 1.53 (d, J = 6.5 Hz, 3H), 0.88-0.81 (m, 4H). |
| 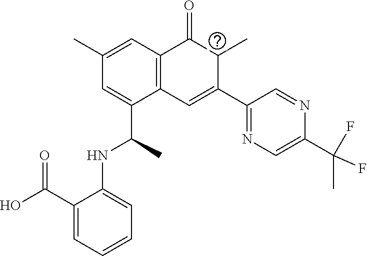 [SMILES: Cc1cc2c(c([C@@H](C)Nc3ccccc3C(=O)O)c1)C=C(c1cnc(C(C)(F)F)cn1)C(C)C2=O] C₂₆H₂₄F₂N₄O₃ | 395 | | ES+ 479.1 [M+1] | ¹H NMR (400 MHz, Methanol-d₄) δ 9.11- 9.02 (m, 2H), 8.16-8.11 (m, 1H), 7.95-7.87 (m, 1H), 7.67 (d, J = 1.9 Hz, 1H), 7.28 (s, 1H), 7.15- 7.06 (m, 1H), 6.57-6.48 (m, 1H), 6.37-6.31 (m, 1H), 5.28-5.18 (m, 1H), 3.58 (s, 3H), 2.45 (s, 3H), 2.21-2.02 (m, 3H), 1.64 (d, J = 6.7 Hz, 3H). |
| 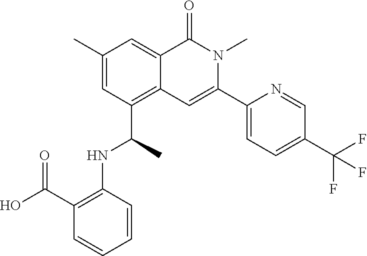 [SMILES: Cc1cc([C@@H](C)Nc2ccccc2C(=O)O)c2cc(-c3ccc(C(F)(F)F)cn3)n(C)c(=O)c2c1] C₂₆H₂₂F₃N₃O₃ | 396 | | ES+ 482.1 [M + H] | ¹H NMR (400 MHz, DMSO-d₆) δ 9.17-9.12 (m, 1H), 8.51 (s, 1H), 8.48- 8.42 (m, 1H), 8.09- 8.00 (m, 2H), 7.84-7.79 (m, 1H), 7.53 (d, J = 1.9 Hz, 1H), 7.19-7.10 (m, 2H), 6.55-6.49 (m, 1H), 6.33 (d, J = 8.4 Hz, 1H), 5.26-5.18 (m, 1H), 3.42 (s, 3H), 2.39 (s, 3H), 1.52 (d, J = 6.5 Hz, 3H). |
| 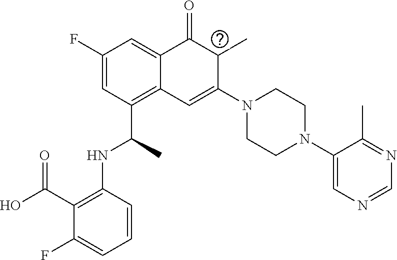 [SMILES: Cc1ncncc1N1CCN(C2=Cc3c(cc(F)cc3[C@@H](C)Nc3cccc(F)c3C(=O)O)C(=O)C2C)CC1] C₂₈H₂₈F₂N₆O₃ | 397 | | ES+ 535.2 [M + H] | ¹H NMR (Methanol-d₄, 400 MHz): δ 8.75 (s, 1H), 8.51 (s, 1H), 7.90-7.77 (m, 1H), 7.61-7.45 (m, 1H), 7.12-7.00 (m, 1H), 6.61 (s, 1H), 6.38-6.24 (m, 1H), 6.12 (d, J = 8.4 Hz, 1H), 5.23-5.10 (m, 1H), 3.72 (s, 3H), 3.33- 3.15 (m, 8H), 2.62 (s, 3H), 1.66 (d, J = 6.6 Hz, 3H). |
| 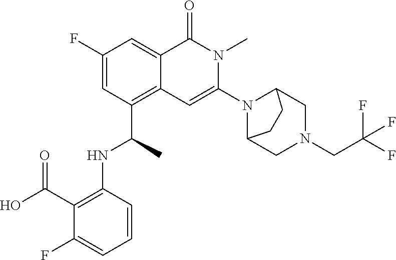 [SMILES: C[C@@H](Nc1cccc(F)c1C(=O)O)c1cc(F)cc2c(=O)n(C)c(N3C4CCC3CN(CC(F)(F)F)C4)cc12] C₂₇H₂₇F₅N₄O₃ | 398 | | ES+ 551.4 [M + H] | ¹H NMR (400 MHz, DMSO-d₆) δ 13.11 (s, 1H), 8.02 (d, J = 6.0 Hz, 1H), 7.73-7.65 (m, 1H), 7.42- 7.35 (m, 1H), 7.21- 7.13 (m, 1H), 6.41-6.32 (m, 1H), 6.23-6.11 (m, 2H), 5.22-5.12 (m, 1H), 3.97-3.80 (m, 2H), 3.58 (s, 3H), 3.40-3.22 (m, 2H), 2.90-2.76 (m, 4H), 2.02-1.76 (m, 4H), 1.54 (d, J = 6.5 Hz, 3H). |
| 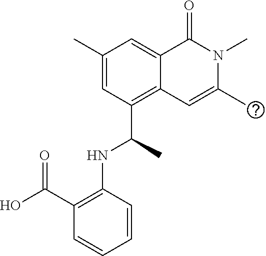 [SMILES: Cc1cc([C@@H](C)Nc2ccccc2C(=O)O)c2cc(C)n(C)c(=O)c2c1] C₂₀H₂₀N₂O₃ | 399 | | ES− 335.1 [M − H] | ¹H NMR (400 MHz, DMSO-d₆) δ 12.73 (s, 1H), 8.42 (s, 1H), 7.97 (s, 1H), 7.83-7.79 (m, 1H), 7.53- 7.44 (m, 2H), 7.20- 7.14 (m, 1H), 6.93 (d, J = 7.6 Hz, 1H), 6.56-6.49 (m, 1H), 6.31 (d, J = 8.5 Hz, 1H), 5.19-5.16 (m, 1H), 3.52 (s, 3H), 2.51 (s, 3H) 1.52 (d, J = 6.5 Hz, 3H). |
| 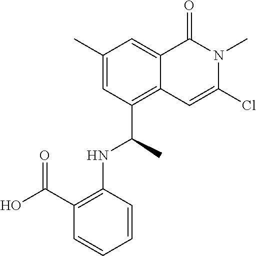 [SMILES: Cc1cc([C@@H](C)Nc2ccccc2C(=O)O)c2cc(Cl)n(C)c(=O)c2c1] C₂₀H₁₉ClN₂O₃ | 400 | | ES+ 371.0 [M + H] | ¹H NMR (400 MHz, DMSO-d₆) δ 12.77 (s, 1H), 8.39 (d, J = 5.0 Hz, 1H), 7.94 (d, J = 1.8 Hz, 1H), 7.83-7.79 (m, 1H), 7.50 (d, J = 1.8 Hz, 1H), 7.27 (s, 1H), 7.22-7.16 (m, 1H), 6.58-6.49 (m, 1H), 6.32 (d, J = 8.4 Hz, 1H), 5.22- 5.19 (m, 1H), 3.66 (s, 3H), 2.35 (s, 3H), 1.50 (d, J = 6.5 Hz, 3H). |
| 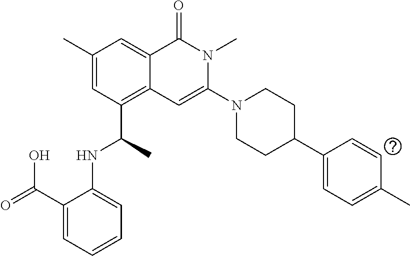 [SMILES: Cc1ccc(C2CCN(c3cc4c([C@@H](C)Nc5ccccc5C(=O)O)cc(C)cc4c(=O)n3C)CC2)cc1] C₃₁H₃₄N₄O₃ | 401 | | ES− 509.3 [M − H] | ¹H NMR (DMSO-d₆, 400 MHz): δ 12.65 (s, 1H), 8.54-8.25 (m, 2H), 7.89 (d, J = 1.8 Hz, 1H), 7.85- 7.75 (m, 1H), 7.70-7.64 (m, 1H), 7.44 (d, J = 1.9 Hz, 1H), 7.25-7.05 (m, 2H), 6.78-6.51 (m, 1H), 6.41 (d, J = 9.6 Hz, 2H), 5.29-5.02 (m, 1H), 3.53 (s, 3H), 2.80-2.73 (m, 3H), 2.51-2.44 (m, 4H), 2.34 (s, 3H), 1.91-1.88 (m, 5H), 1.57 (d, J = 6.6 Hz, 3H). |
| 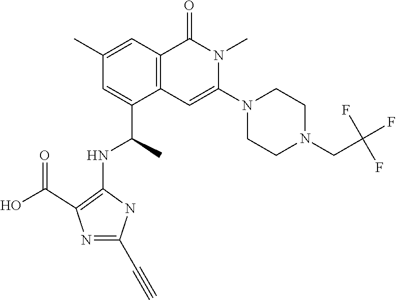 [SMILES: C#Cc1nc(C(=O)O)c(N[C@H](C)c2cc(C)cc3c(=O)n(C)c(N4CCN(CC(F)(F)F)CC4)cc23)[nH]1] C₂₅H₂₆F₃N₅O₃S | 402 | | ES+ 534.2 [M + H] | ¹H NMR (400 MHz, CDCl₃) 8 8.18 (s, 1H), 8.01 (d, J = 5.2 Hz, 1H), 7.42 (s, 1H), 6.11 (s, 1H), 4.77-4.73 (m, 1H), 3.62 (s, 3H), 3.32 (s, 1H), 3.17-3.10 (m, 2H), 3.10-2.65 (m, 8H), 2.44 (s, 3H), 1.73 (d, J = 6.8 Hz, 3H). |
| 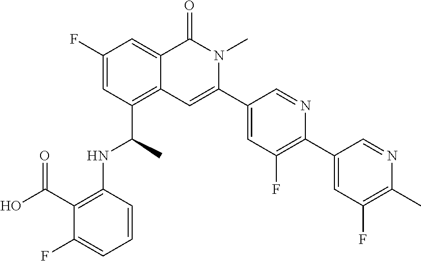 [SMILES: Cc1ncc(-c2ncc(-c3cc4c([C@@H](C)Nc5cccc(F)c5C(=O)O)cc(F)cc4c(=O)n3C)cc2F)cc1F] C₃₀H₂₂F₄N₄O₃ | 403 | | 563.1 [M + H] | ¹H NMR (400 MHz, DMSO-d₆) δ 8.94 (d, J = 1.7 Hz, 1H), 8.88 (d, J = 1.7 Hz, 1H), 8.36-8.28 (m, 1H), 8.23 (s, 1H), 8.21- 8.13 (m, 1H), 7.89-7.81 (m, 1H), 7.54-7.47 (m, 1H), 7.13 (s, 1H), 7.01 (s, 1H), 6.32-6.23 (m, 1H), 6.05 (d, J = 8.4 Hz, 1H), 5.28 (s, 1H), 3.45 (s, 3H), 2.56 (d, J = 2.9 Hz, 3H), 1.50 (d, J = 6.5 Hz, 3H). |
| 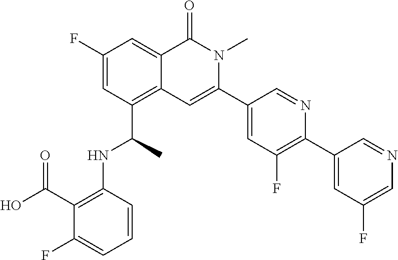 [SMILES: C[C@@H](Nc1cccc(F)c1C(=O)O)c1cc(F)cc2c(=O)n(C)c(-c3cnc(-c4cncc(F)c4)c(F)c3)cc12] C₂₉H₂₀F₄N₄O₃ | 404 | | 547.2 [ [M − H] | ¹H NMR (400 MHz, DMSO-d₆) δ 9.07 (s, 1H), 8.92 (d, J = 2.0 Hz, 1H), 8.76 (d, J = 2.9 Hz, 1H), 8.41-8.33 (m, 2H), 8.27 (d, J = 9.6 Hz, 1H), 7.88- 7.80 (m, 1H), 7.58-7.50 (m, 1H), 7.14 (s, 1H), 6.87 (s, 1H), 6.25-6.16 (m, 1H), 5.99 (d, J = 8.3 Hz, 1H), 5.23 (s, 1H), 3.45 (s, 3H), 1.48 (d, J = 6.5 Hz, 3H). |
| 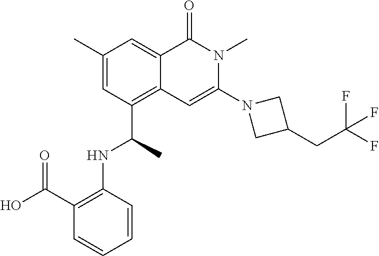 [SMILES: Cc1cc([C@@H](C)Nc2ccccc2C(=O)O)c2cc(N3CC(CC(F)(F)F)C3)n(C)c(=O)c2c1] C₂₅H₂₆F₃N₃O₃ | 405 | | ES+ 474.0 [M + H] | ¹H NMR (400 MHz, DMSO-d₆) δ 12.73 (s, 1H), 8.41-8.35 (m, 1H), 7.84- 7.77 (m, 2H), 7.36 (d, J = 1.9 Hz, 1H), 7.22-7.16 (m, 1H), 6.56-6.48 (m, 1H), 6.36 (d, J = 8.3 Hz, 1H), 5.85 (s, 1H), 5.14- 5.07 (m, 1H), 4.14-4.07 (m, 2H), 3.77-3.69 (m, 2H), 3.42 (s, 3H), 2.98- 2.90 (m, 1H), 2.75-2.65 (m, 2H), 2.29 (s, 3H), 1.54 (d, J = 6.5 Hz, 3H). |
| 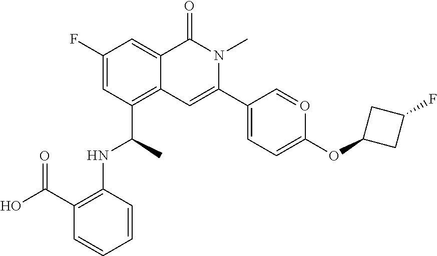 C₂₈H₂₄F₃N₃O₄ | 406 | | ES− 522.4 [M − H] | ¹H NMR (400 MHz, DMSO-d₆) δ 13.22 (s, 1H), 8.42 (d, J = 2.4 Hz, 1H), 8.06 (d, J = 6.0 Hz, 1H), 8.05-8.00 (m, 1H), 7.99- 7.84 (m, 1H), 7.46- 7.43 (m, 1H), 7.16-7.11 (m, 1H), 7.03-6.94 (m, 2H), 6.38-6.33 (m, 1H), 6.11 (d, J = 8.6 Hz, 1H), 5.47-5.27 (m, 3H), 3.37 (s, 3H), 2.79-2.64 (m, 2H), 2.63-2.52 (m, 2H), 1.52 (d, J = 6.5 Hz, 3H). |
| 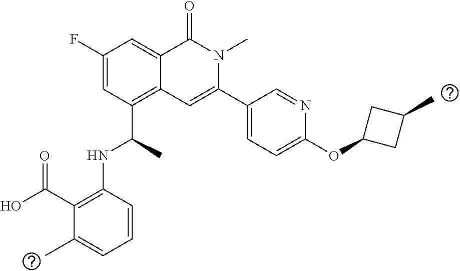 C₂₈H₂₄F₃N₃O₄ | 407 | | ES+ 524.1 [M + H] | ¹H NMR (400 MHz, DMSO-d₆) δ 13.20 (s, 1H), 8.40 (d, J = 2.4 Hz, 1H), 8.07-8.00 (m, 2H), 7.85- 7.82 (m, 1H), 7.46- 7.43 (m, 1H), 7.14-7.12 (m, 1H), 7.01-6.94 (m, 2H), 6.38-6.33 (m, 1H), 6.11 (d, J = 8.6 Hz, 1H), 5.31-5.29 (m, 1H), 5.01- 4.84 (m, 2H), 3.37 (s, 3H), 3.10-2.97 (m, 2H), 2.40-2.22 (m, 2H), 1.52 (d, J = 6.4 Hz, 3H). |
| 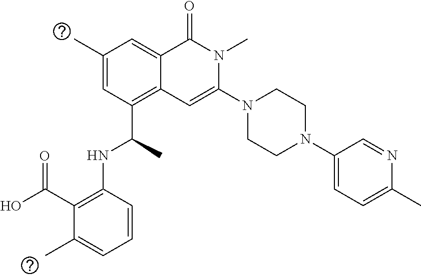 C₂₉H₂₉ClFN₅O₃ | 408 | | ES+ 550.3 [M + H] | ¹H NMR (400 MHz, Methanol-d₄) δ 8.17 (s, 1H), 7.84-7.77 (m, 1H), 7.72-7.64 (m, 1H), 7.60- 7.53 (m, 1H), 7.40 (d, J = 8.8 Hz, 1H), 6.97-6.88 (m, 1H), 6.64 (d, J = 7.9 Hz, 1H), 6.58 (s, 1H), 6.22 (d, J = 8.3 Hz, 1H), 5.14- 5.05 (m, 1H), 3.78 (s, 2H), 3.70 (s, 3H), 3.39 (s, 2H), 3.15 (s, 4H), 2.54 (s, 3H), 1.62 (d, J = 6.6 Hz, 3H). |
| 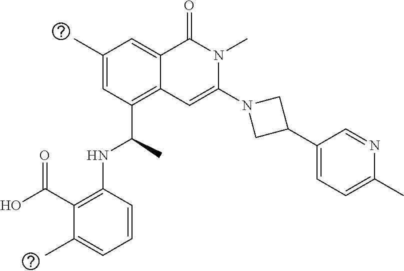 C₂₈H₂₆F₂N₄O₃ | 409 | | ES+ 505.4 [M + H] | ¹H NMR (400 MHz, DMSO-d₆) δ 13.02 (s, 1H), 8.50 (d, J = 2.4 Hz, 1H), 8.09 (s, 1H), 7.80-7.79 (m, 1H), 7.67-7.66 (m, 1H), 7.35-7.33 (m, 1H), 7.28 (d, J = 8.1 Hz, 1H), 7.16-7.14 (m, 1H), 6.35- 6.34 (m, 1H), 6.14 (d, J = 8.6 Hz, 1H), 5.98 (s, 1H), 5.19-5.18 (m, 1H), 4.47- 4.39 (m, 1H), 4.37- 3.98 (m, 1H), 4.05-3.96 (m, 1H), 4.02-3.89 (m, 2H), 3.48 (s, 3H), 2.47 (s, 3H), 1.55 (d, J = 6.5 Hz, 3H). |
| 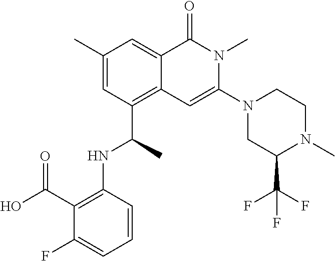 [SMILES: Cc1cc([C@@H](C)Nc2cccc(F)c2C(=O)O)c2cc(N3CCN(C)[C@@H](C(F)(F)F)C3)n(C)c(=O)c2c1] C₂₆H₂₈F₄N₄O₃ | 410 | | ES− 519.4 [M − H] | ¹H NMR (400 MHz, DMSO-d₆) δ 13.16 (s, 1H), 8.16 (s, 1H), 7.89 (d, J = 1.9 Hz, 1H), 7.43 (d, J = 1.9 Hz, 1H), 7.17-7.07 (m, 1H), 6.43 (s, 1H), 6.35- 6.26 (m, 1H), 6.19 (d, J = 8.6 Hz, 1H), 5.20-5.12 (m, 1H), 3.50 (s, 3H), 3.32 (s, 3H), 3.21-3.20 (m, 2H), 3.12-2.75 (m, 4H), 2.64-2.63 (m, 1H), 2.34 (s, 3H), 1.52 (d, J = 6.5 Hz, 3H). |
| 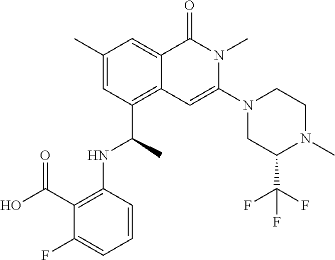 [SMILES: Cc1cc([C@@H](C)Nc2cccc(F)c2C(=O)O)c2cc(N3CCN(C)[C@H](C(F)(F)F)C3)n(C)c(=O)c2c1] C₂₆H₂₈F₄N₄O₃ | 411 | | ES− 519.4 [M − H] | ¹H NMR (400 MHz, DMSO-d₆) δ 13.21 (s, 1H), 8.13 (s, 1H), 7.90 (d, J = 1.9 Hz, 1H), 7.43 (d, J = 1.9 Hz, 1H), 7.20-7.09 (m, 1H), 6.44 (s, 1H), 6.36- 6.28 (m, 1H), 6.20 (d, J = 8.6 Hz, 1H), 5.20-5.12 (s, 1H), 3.50 (s, 3H), 3.32- 3.23 (m, 5H), 3.15-2.75 (m, 4H), 3.24 (d, J = 10.7 Hz, 1H), 2.34 (s, 3H), 1.53 (d, J = 6.5 Hz, 3H). |
| 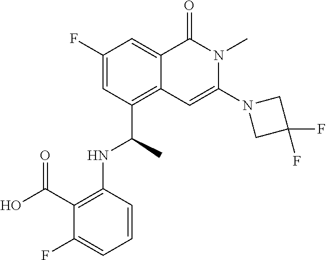 [SMILES: C[C@@H](Nc1cccc(F)c1C(=O)O)c1cc(F)cc2c(=O)n(C)c(N3CC(F)(F)C3)cc12] C₂₂H₁₉F₄N₃O₃ | 412 | | ES+ 449.9 [M + H] | ¹H NMR (400 MHz, Methanol-d₄) δ 7.79- 7.71 (m, 1H), 7.51-7.43 (m, 1H), 7.06-6.96 (m, 1H), 6.36-6.17 (m, 2H), 6.06 (d, J = 8.4 Hz, 1H), 5.17-5.07 (m, 1H), 4.49- 4.33 (m, 4H), 3.58 (s, 3H), 1.62 (d, J = 6.7 Hz, 3H). |
| 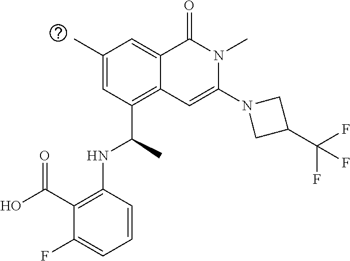 C₂₃H₂₀F₅N₃O₃ | 413 | | ES+ 482.3 [M + H] | ¹H NMR (400 MHz, DMSO-d₆) δ 13.23 (s, 1H), 8.08 (d, J = 5.9 Hz, 1H), 7.76-7.58 (m, 1H), 7.39- 7.29 (m, 1H), 7.26- 7.08 (m, 1H), 6.46-6.26 (m, 1H), 6.12 (d, J = 8.5 Hz, 1H), 5.99 (s, 1H), 5.28- 5.16 (m, 1H), 4.45- 4.16 (m, 2H), 4.10-4.04 (m, 2H), 3.88-3.52 (m, 1H), 3.43 (s, 3H), 1.53 (d, J = 6.4 Hz, 3H). |
| 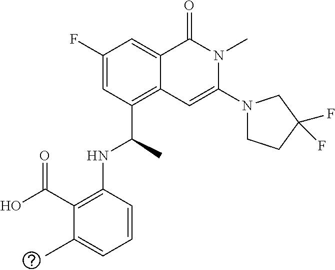 C₂₃H₂₁F₄N₃O₃ | 414 | | ES+ 464.0 [M + H] | ¹H NMR (400 MHz, Chloroform-d) δ 8.45 (s, 1H), 8.00-7.94 (m, 1H), 7.43-7.37 (m, 1H), 7.17- 7.09 (m, 1H), 6.42- 6.34 (m, 1H), 6.24 (s, 1H), 6.04 (d, J = 8.6 Hz, 1H), 4.99-4.90 (m, 1H), 3.66 (s, 3H), 3.57-3.49 (m, 2H), 3.44-3.38 (m, 2H), 2.61-2.47 (m, 2H), 1.69 (d, J = 6.7 Hz, 3H). |
| 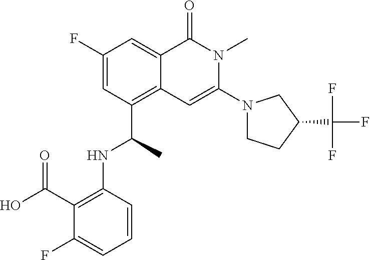 [SMILES: C[C@@H](Nc1cccc(F)c1C(=O)O)c1cc(F)cc2c(=O)n(C)c(N3CC[C@@H](C(F)(F)F)C3)cc12] C₂₄H₂₂F₅N₃O₃ | 415 | | ES+ 496.2 [M + H] | ¹H NMR (400 MHz, DMSO-d₆) δ 13.23 (s, 1H), 8.08 (d, J = 6.0 Hz, 1H), 7.77-7.63 (m, 1H), 7.45- 7.30 (m, 1H), 7.23- 7.08 (m, 1H), 6.41 (s, 1H), 6.39-6.31 (m, 1H), 6.13 (d, J = 8.6 Hz, 1H), 5.25- 5.20 (m, 1H), 3.51 (s, 3H), 3.40-3.35 (m, 3H), 3.29- 3.20 (m, 2H), 2.36- 2.21 (m, 1H), 2.11-1.93 (m, 1H), 1.54 (d, J = 6.5 Hz, 3H). |
| 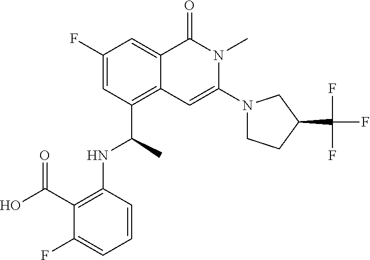 [SMILES: C[C@@H](Nc1cccc(F)c1C(=O)O)c1cc(F)cc2c(=O)n(C)c(N3CC[C@H](C(F)(F)F)C3)cc12] C₂₄H₂₂F₅N₃O₃ | 416 | | ES+ 496.2 [M + H] | ¹H NMR (400 MHz, DMSO-d₆) δ 13.23 (s, 1H), 8.07 (d, J = 6.0 Hz, 1H), 7.78-7.63 (m, 1H), 7.47- 7.33 (m, 1H), 7.24- 7.07 (m, 1H), 6.41 (s, 1H), 6.39-6.31 (m, 1H), 6.14 (d, J = 8.5 Hz, 1H), 5.25- 5.20 (m, 1H), 3.51 (s, 3H), 3.40-3.35 (m, 3H), 3.29- 3.16 (m, 2H), 2.39- 2.18 (m, 1H), 2.10-1.98 (m, 1H), 1.54 (d, J = 6.5 Hz, 3H). |
| 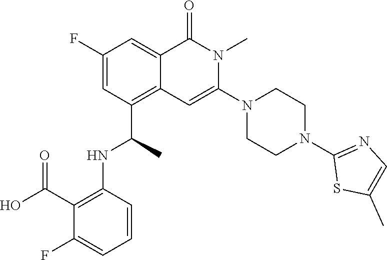 [SMILES: Cc1cnc(N2CCN(c3cc4c([C@@H](C)Nc5cccc(F)c5C(=O)O)cc(F)cc4c(=O)n3C)CC2)s1] C₂₇H₂₇F₂N₅O₃S | 417 | | ES+ 540.2 [M + H] | ¹H NMR (400 MHz, DMSO-d₆) δ 8.19 (s, 1H), 7.72-7.70 (m, 1H), 7.42- 7.40 (m, 1H), 7.06 (s, 1H), 6.88 (d, J = 1.4 Hz, 1H), 6.47 (s, 1H), 6.35- 6.25 (m, 1H), 6.09 (d, J = 8.4 Hz, 1H), 5.21-5.15 (m, 1H), 3.90-3.57 (m, 5H), 3.30 (s, 3H), 3.29- 2.90 (m, 3H), 2.25 (s, 3H), 1.53 (d, J = 6.6 Hz, 3H). |
| 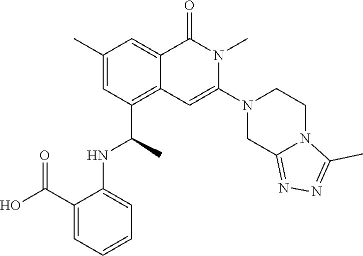 [SMILES: Cc1cc([C@@H](C)Nc2ccccc2C(=O)O)c2cc(N3CCn4c(C)nnc4C3)n(C)c(=O)c2c1] C₂₆H₂₈N₆O₃ | 418 | | ES+ 473.4 [M + H] | ¹H NMR (400 MHz, DMSO-d₆) δ 12.76 (s, 1H), 8.39 (d, J = 6.0 Hz, 1H), 7.90 (s, 1H), 7.84-7.76 (m, 1H), 7.43 (d, J = 1.9 Hz, 1H), 7.24-7.12 (m, 1H), 6.58 (s, 1H), 6.56- 6.49 (m, 1H), 6.38 (d, J = 8.5 Hz, 1H), 5.24-5.16 (m, 1H), 4.43 (s, 2H), 4.11 (s, 2H), 3.53 (s, 3H), 3.45- 3.40 (m, 2H), 2.35 (s, 3H), 2.30 (s, 3H), 1.54 (d, J = 6.5 Hz, 3H). |
| 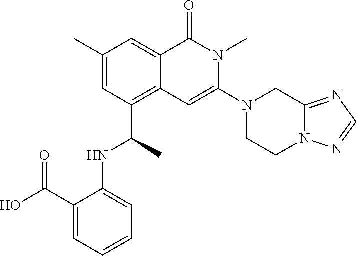 [SMILES: Cc1cc([C@@H](C)Nc2ccccc2C(=O)O)c2cc(N3CCn4ncnc4C3)n(C)c(=O)c2c1] C₂₅H₂₆N₆O₃ | 419 | | ES− 457.2 [M − H] | ¹H NMR (400 MHz, DMSO-d₆) δ 12.73 (s, 1H), 8.40 (d, J = 6.1 Hz, 1H), 8.02 (s, 1H), 7.91 (s, 1H), 7.85-7.75 (m, 1H), 7.44 (d, J = 2.0 Hz, 1H), 7.29- 7.08 (m, 1H), 6.61 (s, 1H), 6.57-6.50 (m, 1H), 6.38 (d, J = 8.5 Hz, 1H), 5.31- 5.09 (m, 1H), 4.49 (d, J = 3.6 Hz, 2H), 4.35 (s, 2H), 3.55 (s, 3H), 3.40-3.35 (m, 2H), 2.34 (s, 3H), 1.54 (d, J = 6.5 Hz, 3H). |
| 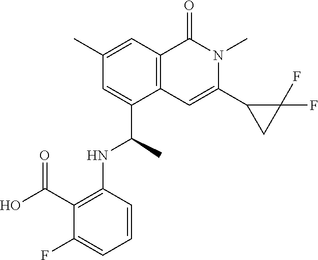 [SMILES: Cc1cc([C@@H](C)Nc2cccc(F)c2C(=O)O)c2cc(C3CC3(F)F)n(C)c(=O)c2c1] C₂₃H₂₁F₃N₂O₃ | 420 | diastereomer 1 | ES+ 453.1 [M + Na] | ¹H NMR (400 MHz, Methanol-d₄) δ 8.04 (s, 1H), 7.73 (s, 1H), 7.01 (s, 1H), 6.88-6.76 (m, 1H), 6.29-6.19 (m, 1H), 5.95 (d, J = 8.4 Hz, 1H), 5.06- 5.04 (m, 1H), 3.74 (s, 3H), 3.19-3.08 (m, 1H), 2.42 (s, 3H), 2.17-2.01 (m, 2H), 1.57 (d, J = 6.8 Hz, 3H). |
| 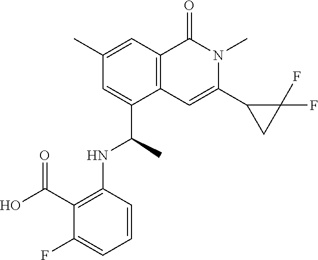 [SMILES: Cc1cc([C@@H](C)Nc2cccc(F)c2C(=O)O)c2cc(C3CC3(F)F)n(C)c(=O)c2c1] C₂₃H₂₁F₃N₂O₃ | 421 | diastereomer 2 | ES+ 453.2 [M + Na] | ¹H NMR (400 MHz, Methanol-d₄) δ 8.01 (s, 1H), 7.70 (s, 1H), 7.01 (s, 1H), 6.86-6.74 (m, 1H), 6.28-6.14 (m, 1H), 5.92 (d, J = 8.4 Hz, 1H), 5.14- 5.02 (q, J = 6.4 Hz, 1H), 3.72 (s, 3H), 3.16-3.05 (m, 1H), 2.39 (s, 3H), 2.16- 2.00 (m, 2H), 1.55 (d, J = 6.8 Hz, 3H). |
| 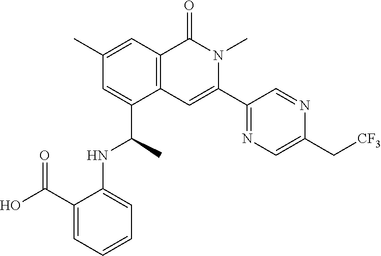 [SMILES: Cc1cc([C@@H](C)Nc2ccccc2C(=O)O)c2cc(-c3cnc(CC(F)(F)F)cn3)n(C)c(=O)c2c1] C₂₆H₂₃F₃N₄O₃ | 422 | | ES+ 497.2 [M + H] | ¹H NMR (400 MHz, DMSO-d₆) δ 12.69 (s, 1H), 9.07 (s, 1H), 8.87 (s, 1H), 8.40 (s, 1H), 8.03 (s, 1H), 7.80 (d, J = 7.9 Hz, 1H), 7.52 (s, 1H), 7.24 (s, 1H), 7.20-7.13 (m, 1H), 6.56- 6.48 (m, 1H), 6.37 (d, J = 8.5 Hz, 1H), 5.31 (s, 1H), 4.09 (d, J = 11.2 Hz, 1H), 4.04 (d, J = 11.3 Hz, 1H), 3.44 (s, 3H), 2.39 (s, 3H), 1.52 (d, J = 6.5 Hz, 3H). |
| 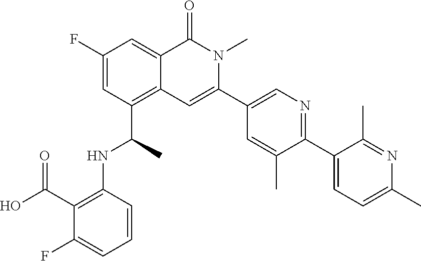 C₃₁H₂₅F₃N₄O₃ | 423 | | ES+ 559.4 [M + H] | ¹H NMR (400 MHz, DMSO-d₆) δ 13.25 (s, 1H), 8.83 (s, 1H), 8.26 (d, J = 10.6 Hz, 1H), 8.08 (s, 1H), 7.87 (d, J = 8.6 Hz, 1H), 7.74 (d, J = 7.8 Hz, 1H), 7.48 (d, J = 9.4 Hz, 1H), 7.26 (d, J = 7.8 Hz, 1H), 7.15 (s, 2H), 6.41-6.31 (m, 1H), 6.12 (d, J = 8.5 Hz, 1H), 5.35 (s, 1H), 3.46 (s, 3H), 2.53 (s, 3H), 2.43 (s, 3H), 1.54 (d, J = 6.4 Hz, 3H). |
| 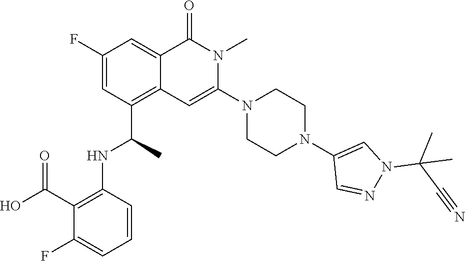 [SMILES: C[C@@H](Nc1cccc(F)c1C(=O)O)c1cc(F)cc2c(=O)n(C)c(N3CCN(c4cnn(C(C)(C)C#N)c4)CC3)cc12] C₃₀H₃₁F₂N₇O₃ | 424 | | ES+ 576.2 [M + H] | ¹H NMR (400 MHz, Methanol-d₄) δ 7.83- 7.80 (m, 1H), 7.68 (d, J = 2.7 Hz, 1H), 7.45-7.40 (m, 1H), 7.14-7.11 (m, 1H), 6.55 (s, 1H), 6.40- 6.31 (m, 1H), 6.15 (d, J = 8.6 Hz, 1H), 5.95 (d, J = 2.7 Hz, 1H), 5.19-5.14 (m, 1H), 4.00-3.69 (m, 5H), 3.40-2.90 (m, 6H), 1.97 (s, 6H), 1.66 (d, J = 6.7 Hz, 3H). |
| 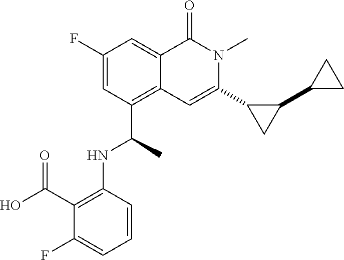 C₂₅H₂₄F₂N₂O₃ | 425 | trans cyclopropyl diastereomer 1 | ES+ 439.2 [M + H] | ¹H NMR (400 MHz, DMSO-d₆) δ 13.11(s, 1H), 8.20 (s, 1H), 7.77-7.69 (m, 1H), 7.47-7.37 (m, 1H), 7.07-6.95 (m, 1H), 6.61 (s, 1H), 6.32-6.23 (m, 1H), 6.04 (d, J = 8.0 Hz, 1H), 5.22-5.15 (m, 1H), 3.70 (s, 3H), 1.94- 1.85 (m, 1H), 1.48 (d, J = 4.0 Hz, 3H), 1.15-0.79 (m, 4H), 0.52-0.18 (m, 4H). |
| 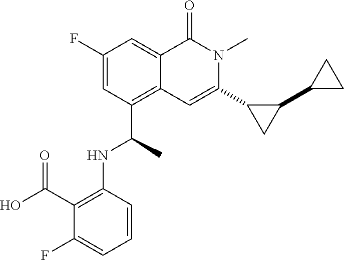 C₂₅H₂₄F₂N₂O₃ | 426 | trans cyclopropyl diastereomer 2 | ES+ 439.2 [M + H] | ¹H NMR (400 MHz, DMSO-d₆) δ 13.11(s, 1H), 8.19 (s, 1H), 7.77-7.69 (m, 1H), 7.45-7.36 (m, 1H), 7.07-6.96 (m, 1H), 6.63 (s, 1H), 6.33-6.24 (m, 1H), 6.04 (d, J = 8.5 Hz, 1H), 5.22-5.15 (m, 1H), 3.70 (s, 3H), 1.93- 1.84 (m, 1H), 1.47 (d, J = 6.5 Hz, 3H), 1.17-0.74 (m, 4H), 0.55-0.16 (m, 4H). |
| 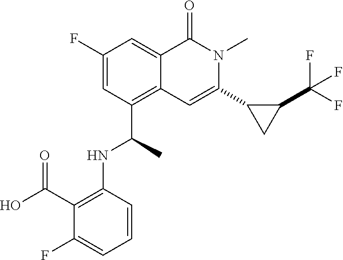 C₂₃H₁₉F₅N₂O₃ | 427 | trans cyclopropyl diastereomer 1 | ES− 465.3 [M − H] | ¹H NMR (400 MHz, DMSO-d₆) δ 13.29 (s, 1H), 8.15-8.11 (m, 1H), 7.77- 7.74 (m, 1H), 7.43- 7.40 (m, 1H), 7.12-7.06 (m, 1H), 6.80 (s, 1H), 6.35- 6.30 (m, 1H), 6.09 (d, J = 8.5 Hz, 1H), 5.37-5.35 (m, 1H), 3.68 (s, 3H), 2.72- 2.67 (m, 2H), 1.53- 1.41 (m, 5H). |
| 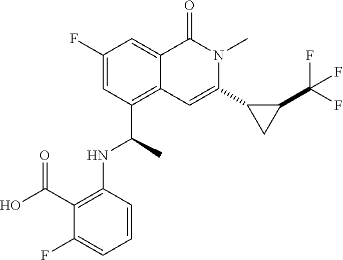 C₂₃H₁₉F₅N₂O₃ | 428 | trans cyclopropyl diastereomer 2 | ES− 465.3 [M − H] | ¹H NMR (400 MHz, DMSO-d₆) δ 13.15 (s, 1H), 8.15-8.14 (m, 1H), 7.77- 7.74 (m, 1H), 7.45- 7.42 (m, 1H), 7.09-7.01 (m, 1H), 6.78 (s, 1H), 6.32- 6.27 (m, 1H), 6.05 (d, J = 8.5 Hz, 1H), 5.31-5.30 (m, 1H), 3.68 (s, 3H), 2.73- 2.70 (m, 2H), 1.67- 1.42 (m, 5H). |
| 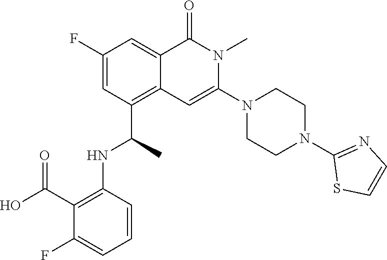 [SMILES: C[C@@H](Nc1cccc(F)c1C(=O)O)c1cc(F)cc2c(=O)n(C)c(N3CCN(c4nccs4)CC3)cc12] C₂₆H₂₅F₂N₅O₃S | 429 | | ES+ 526.0 [M + H] | ¹H NMR (400 MHz, DMSO-d₆) δ 13.21 (s, 1H), 8.10 (d, J = 6.0 Hz, 1H), 7.75-7.70 (m, 1H), 7.42- 7.31 (m, 1H), 7.22 (d, J = 3.6 Hz, 1H), 7.17-7.01 (m, 1H), 6.92 (d, J = 3.6 Hz, 1H), 6.48 (s, 1H), 6.39- 6.20 (m, 1H), 6.14 (d, J = 8.6 Hz, 1H), 5.25-5.10 (m, 1H), 4.01-3.75 (m, 2H), 3.57 (s, 3H), 3.32 (s, 3H), 3.15 (s, 3H), 1.55 (d, J = 6.5 Hz, 3H). |
| 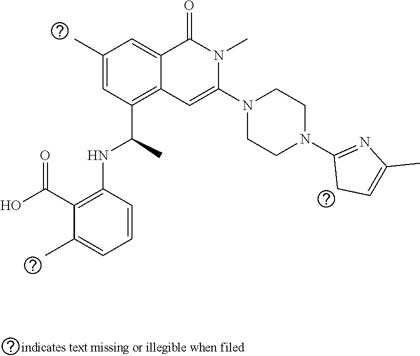 C₂₇H₂₇F₂N₅O₃S | 430 | | ES+ 540.4 [M + H] | ¹H NMR (400 MHz, DMSO-d₆) δ 8.20 (s, 1H), 7.74-7.69 (m, 1H), 7.45- 7.38 (m, 1H), 7.07- 7.01 (m, 1H), 6.49-6.42 (m, 2H), 6.33-6.24 (m, 1H), 6.07 (d, J = 8.5 Hz, 1H), 5.19-5.10 (m, 1H), 3.97-3.96 (m, 1H), 3.56 (s, 3H), 3.32-3.28 (m, 3H), 3.22-2.75 (m, 4H), 2.17 (s, 3H), 1.52 (d, J = 6.6 Hz, 3H). |
| 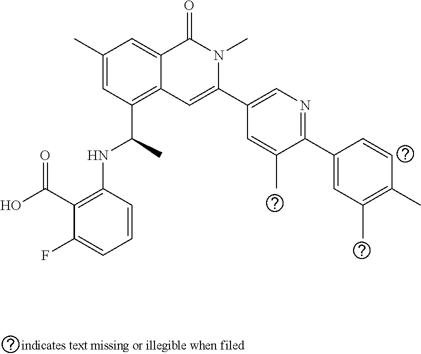 [SMILES: Cc1cc([C@@H](C)Nc2cccc(F)c2C(=O)O)c2cc(-c3cnc(-c4ccc(C)c(C)c4)c(C)c3)n(C)c(=O)c2c1] C₃₁H₂₅F₃N₄O₃ | 431 | | ES− 557.4 [M − H] | ¹H NMR (400 MHz, DMSO-d₆) δ 13.11 (s, 1H), 8.95 (d, J = 1.7 Hz, 1H), 8.87 (d, J = 1.7 Hz, 1H), 8.32-8.28 (m, 1H), 8.21- 8.13 (m, 2H), 8.03 (s, 1H), 7.52 (d, J = 1.9 Hz, 1H), 7.11-7.08 (m, 2H), 6.38-6.30 (m, 1H), 6.15 (d, J = 8.6 Hz, 1H), 5.25- 5.15 (m, 1H), 3.45 (s, 3H), 2.57 (d, J = 2.9 Hz, 3H), 2.41 (s, 3H), 1.51 (d, J = 6.5 Hz, 3H). |
| 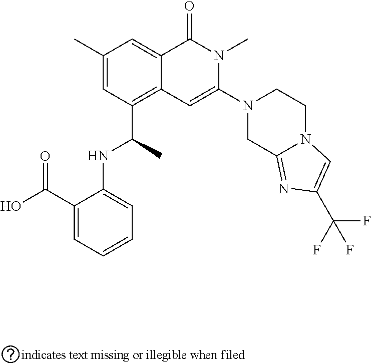 [SMILES: Cc1cc([C@@H](C)Nc2ccccc2C(=O)O)c2cc(N3CCn4cc(C(F)(F)F)nc4C3)n(C)c(=O)c2c1] C₂₇H₂₆F₃N₅O₃ | 432 | | ES− 524.3 [M − H] | ¹H NMR (400 MHz, Methanol-d₄) δ 8.02 (d, J = 1.7 Hz, 1H), 7.91-7.90 (m, 1H), 7.66 (d, J = 1.4 Hz, 1H), 7.59 (d, J = 1.7 Hz, 1H), 7.11-7.10 (m, 1H), 6.71 (s, 1H), 6.52- 6.50 (m, 1H), 6.36-6.30 (m, 1H), 5.11-5.09 (m, 1H), 4.35-4.15 (m, 3H), 3.69 (s, 3H), 3.33 (s, 3H), 2.40 (s, 3H), 1.63 (d, J = 6.7 Hz, 3H). |
| 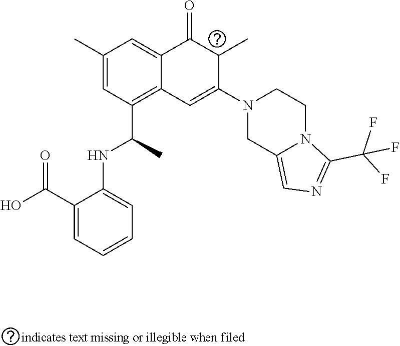 [SMILES: Cc1cc2c(c([C@@H](C)Nc3ccccc3C(=O)O)c1)C=C(N1CCn3c(cnc3C(F)(F)F)C1)C(C)C2=O] C₂₇H₂₆F₃N₅O₃ | 433 | | ES+ 526.0 [M + H] | ¹H NMR (400 MHz, DMSO-d₆) δ 12.72 (s, 1H), 8.40 (d, J = 6.0 Hz, 1H), 7.91 (d, J = 1.9 Hz, 1H), 8.31-7.42 (m, 1H), 7.45 (d, J = 1.9 Hz, 1H), 7.21- 7.17 (m, 1H), 7.04 (s, 1H), 6.58-6.50 (m, 2H), 6.38 (d, J = 8.5 Hz, 1H), 5.16- 5.13 (m, 1H), 4.39-4.33 (m, 4H), 3.54-3.45 (m, 5H), 2.34 (s, 3H), 1.53 (d, J = 6.5 Hz, 3H). |
| 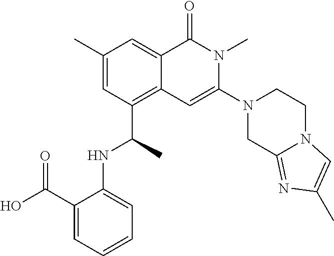 [SMILES: Cc1cc([C@@H](C)Nc2ccccc2C(=O)O)c2cc(N3CCn4cc(C)nc4C3)n(C)c(=O)c2c1] C₂₇H₂₉N₅O₃ | 434 | | ES+ 472.3 [M + H] | ¹H NMR (400 MHz, Methanol-d₄) δ 8.00 (s, 1H), 7.92 (d, J = 7.9 Hz, 1H), 7.60 (s, 1H), 7.09- 7.01 (m, 1H), 6.82 (s, 1H), 6.65 (s, 1H), 6.54-6.45 (m, 1H), 6.30 (d, J = 5.2 Hz, 1H), 5.04-5.00 (m, 1H), 4.27-4.00 (m, 4H), 3.68 (s, 3H), 3.64 (s, 2H), 2.37 (s, 3H), 2.20 (s, 3H), 1.61 (d, J = 6.7 Hz, 3H). |
| 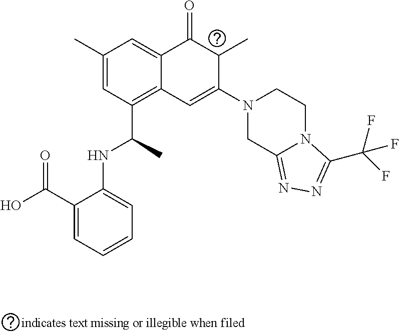 [SMILES: Cc1cc2c(c([C@@H](C)Nc3ccccc3C(=O)O)c1)C=C(N1CCn3c(nnc3C(F)(F)F)C1)C(C)C2=O] C₂₆H₂₅F₃N₆O₃ | 435 | | ES− 525.4 [M − H] | ¹H NMR (DMSO-d₆, 400 MHz, ppm): δ 8.54 (s, 1H), 7.90 (s, 1H), 7.84-7.77 (m, 1H), 7.44 (d, J = 1.9 Hz, 1H), 7.20-7.10 (m, 1H), 6.61 (s, 1H), 6.55- 6.45 (m, 1H), 6.35 (d, J = 8.5 Hz, 1H), 5.20-5.10 (m, 1H), 4.61-4.38 (m, 4H), 3.55 (s, 3H), 3.35 (s, 2H), 2.33 (s, 3H), 1.53 (d, J = 6.5 Hz, 3H). |
| 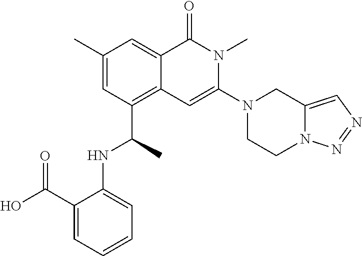 [SMILES: Cc1cc([C@@H](C)Nc2ccccc2C(=O)O)c2cc(N3CCn4nncc4C3)n(C)c(=O)c2c1] C₂₅H₂₆N₆O₃ | 436 | | ES− 457.3 [M − H] | ¹H NMR (400 MHz, Chloroform-d) δ 8.16 (s, 1H), 8.00 (d, J = 7.9 Hz, 1H), 7.70 (s, 1H), 7.56 (s, 1H), 7.28-7.19 (m, 1H), 6.67 (s, 2H), 6.51 (d, J = 8.2 Hz, 1H), 4.95-4.94 (m, 1H), 4.67-4.59 (m, 2H), 4.38-4.24 (m, 2H), 3.63 (s, 3H), 3.60-3.58 (m, 2H), 2.48 (s, 3H), 1.73 (d, J = 6.6 Hz, 3H). |
| 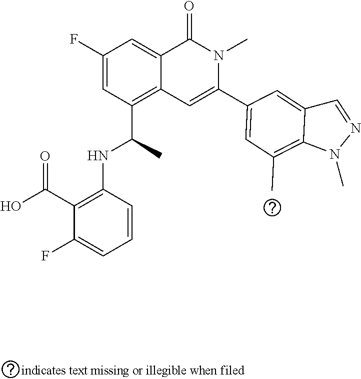 [SMILES: Cc1cc(-c2cc3c([C@@H](C)Nc4cccc(F)c4C(=O)O)cc(F)cc3c(=O)n2C)cc2cnn(C)c12] C₂₇H₂₁F₃N₄O₃ | 437 | | ES− 505.2 [M − H] | ¹H NMR (400 MHz, DMSO-d₆) δ 8.25 (d, J = 2.3 Hz, 1H), 8.16 (s, 1H), 7.87-7.80 (m, 2H), 7.59- 7.53 (m, 1H), 7.50- 7.45 (m, 1H), 7.05 (d, J = 8.4 Hz, 1H), 6.94 (s, 1H), 6.35-6.26 (m, 1H), 6.06 (d, J = 8.5 Hz, 1H), 5.23- 5.15 (m, 1H), 4.24 (s, 3H), 3.37 (s, 3H), 1.50 (d, J = 6.5 Hz, 3H). |
| 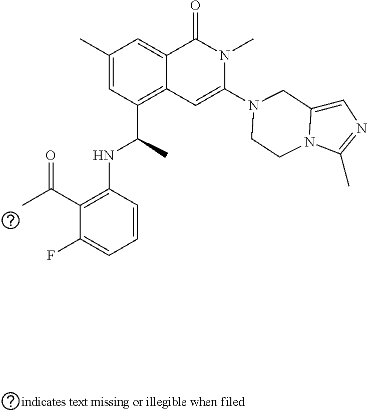 [SMILES: CC(=O)c1c(F)cccc1N[C@H](C)c1cc(C)cc2c(=O)n(C)c(N3CCn4c(cnc4C)C3)cc12] C₂₇H₂₈FN₅O₃ | 438 | | ES+ 490.2 [M + H] | ¹H NMR (400 MHz, Methanol-d₄) δ 8.02 (s, 1H), 7.64 (d, J = 1.8 Hz, 1H), 7.01-6.98 (m, 2H), 6.73 (s, 1H), 6.29-6.27 (m, 1H), 6.13 (d, J = 8.4 Hz, 1H), 5.05-4.98 (m, 1H), 4.40-4.18 (m, 4H), 3.67 (s, 4H), 3.33 (s, 1H), 2.50 (s, 3H), 2.42 (s, 3H), 1.62 (d, J = 6.7 Hz, 3H).5 |
| 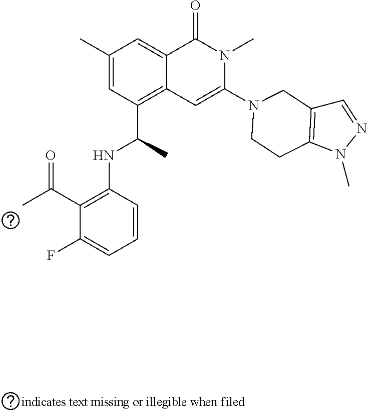 [SMILES: CC(=O)c1c(F)cccc1N[C@H](C)c1cc(C)cc2c(=O)n(C)c(N3CCc4c(cnn4C)C3)cc12] C₂₇H₂₈FN₅O₃ | 439 | | ES+ 490.1 [M + H] | ¹H NMR (400 MHz, DMSO-d₆) δ 13.11 (s, 1H), 8.13-8.12 (m, 1H), 7.89 (s, 1H), 7.43-7.42 (m, 1H), 7.17-7.13 (m, 2H), 6.46 (s, 1H), 6.36-6.31 (m, 1H), 6.22-6.20 (m, 1H), 5.14-5.11 (m, 1H), 4.05-3.97 (m, 2H), 3.72 (s, 3H), 3.54-3.50 (m, 3H), 3.31 (s, 2H), 2.92- 2.87 (m, 2H), 2.34 (s, 3H), 1.53 (d, J = 6.5 Hz, 3H). |
| 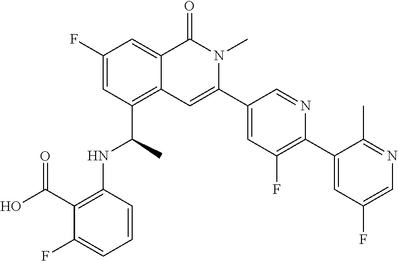 [SMILES: Cc1ncc(F)cc1-c1ncc(-c2cc3c([C@@H](C)Nc4cccc(F)c4C(=O)O)cc(F)cc3c(=O)n2C)cc1F] C₃₀H₂₂F₄N₄O₃ | 440 | | ES+ 563.2 [M + H] | ¹H NMR (400 MHz, DMSO-d₆) δ 8.87 (s, 1H), 8.64 (d, J = 2.9 Hz, 1H), 8.34-8.31 (m, 1H), 8.09 (s, 1H), 7.89-7.85 (m, 2H), 7.50-7.47 (m, 1H), 7.13 (d, J = 7.4 Hz, 2H), 6.38-6.33 (m, 1H), 6.12 (d, J = 8.6 Hz, 1H), 5.34 (s, 1H), 3.46 (s, 3H), 2.46 (s, 3H), 1.54 (d, J = 6.6 Hz, 3H). |
| 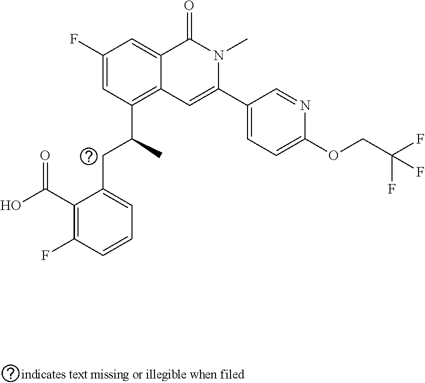 [SMILES: C[C@@H](Cc1cccc(F)c1C(=O)O)c1cc(F)cc2c(=O)n(C)c(-c3ccc(OCC(F)(F)F)nc3)cc12] C₂₆H₂₀F₅N₃O₄ | 441 | | ES− 532.2 [M − H] | ¹H NMR (400 MHz, DMSO-d₆) δ 8.48 (d, J = 2.5 Hz, 1H), 8.28 (s, 1H), 8.14-8.09 (m, 1H), 7.87- 7.80 (m, 1H), 7.51-7.44 (m, 1H), 7.17 (d, J = 8.6 Hz, 1H), 7.09-7.01 (m, 1H), 6.98 (s, 1H), 6.35- 6.27 (m, 1H), 6.07 (d, J = 8.5 Hz, 1H), 5.27 (d, J = 6.8 Hz, 1H), 5.13-5.05 (m, 2H), 3.36 (s, 3H), 1.50 (d, J = 6.5 Hz, 3H) |
| 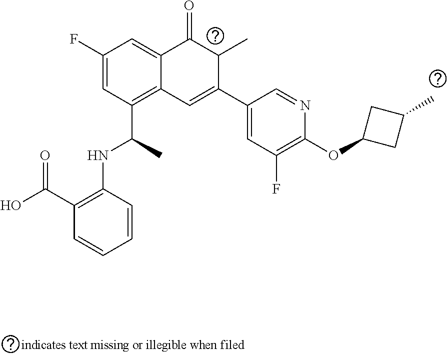 C₂₈H₂₄F₃N₃O₄ | 442 | | ES+ 524.3 [M + H] | ¹H NMR (400 MHz, DMSO-d₆) δ 8.40 (d, J = 5.9 Hz, 1H), 8.25 (d, J = 2.0 Hz, 1H), 8.13-8.11 (m, 1H), 7.86-7.83 (m, 2H), 7.45-7.43 (m, 1H), 7.22-7.13 (m, 1H), 7.02 (s, 1H), 6.58-6.55 (m, 1H), 6.30 (d, J = 8.5 Hz, 1H), 5.56-5.49 (m, 2H), 5.36-5.31 (m, 1H), 3.38 (s, 3H), 2.82-2.49 (m, 4H), 1.53 (d, J = 6.5 Hz, 3H). |
| 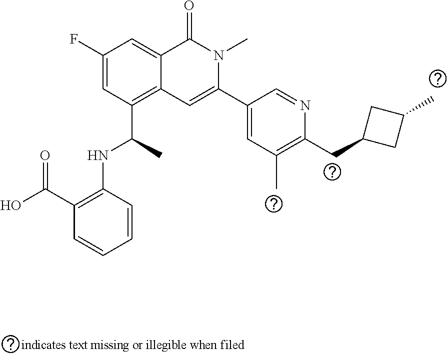 [SMILES: Cc1cc(-c2cc3c([C@@H](C)Nc4ccccc4C(=O)O)cc(F)cc3c(=O)n2C)cnc1C[C@H]1C[C@H](C)C1] C₂₈H₂₄F₃N₃O₄ | 443 | | ES− 522.3 [M − H] | ¹H NMR (400 MHz, DMSO-d₆) δ 12.82 (s, 1H), 8.41 (s, 1H), 8.24 (d, J = 2.0 Hz, 1H), 8.14-8.09 (m, 1H), 7.86-7.80 (m , 2H), 7.45-7.40 (m, 1H), 7.21- 7.14 (m, 1H), 7.03 (s, 1H), 6.58-6.52 (m, 1H), 6.30 (d, J = 8.5 Hz, 1H), 5.32 (s, 1H), 5.08-4.98 (m, 1H), 4.96-4.86 (m, 1H), 3.38 (s, 3H), 3.11-3.00 (m, 2H), 2.45-2.30 (m, 2H), 1.53 (d, J = 6.5 Hz, 3H). |
| 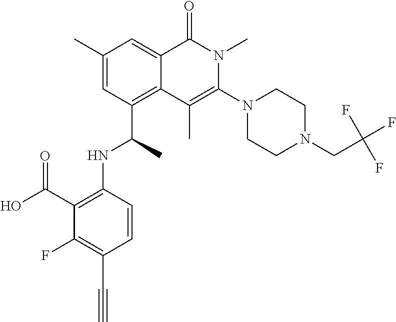 [SMILES: C#Cc1ccc(N[C@H](C)c2cc(C)cc3c(=O)n(C)c(N4CCN(CC(F)(F)F)CC4)c(C)c23)c(C(=O)O)c1F] C₂₈H₂₈F₄N₄O₃ | 444 | | ES− 543.3 [M − H] | ¹H NMR (400 MHz, DMSO-d₆) δ 13.50 (s, 1H), 8.32 (d, J = 6.1 Hz, 1H), 7.89 (s, 1H), 7.39 (d, J = 1.9 Hz, 1H), 7.33-7.25 (m, 1H), 6.37 (s, 1H), 6.20 (d, J = 9.0 Hz, 1H), 5.25- 5.16 (m, 1H), 4.14-4.10 (m, 1H), 3.49 (s, 3H), 3.32- 3.23 (m, 2H), 3.17- 2.82 (m, 8H), 2.33 (s, 3H), 1.53 (d, J = 6.5 Hz, 3H). |
| 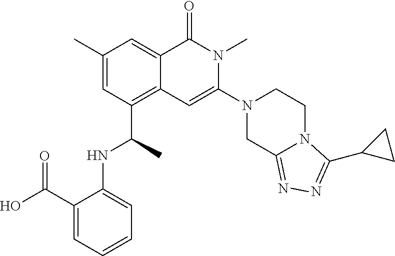 [SMILES: Cc1cc([C@@H](C)Nc2ccccc2C(=O)O)c2cc(N3CCn4c(nnc4C4CC4)C3)n(C)c(=O)c2c1] C₂₈H₃₀N₆O₃ | 445 | | ES− 497.3 [M − H] | ¹H NMR (400 MHz, DMSO-d₆) δ 8.48 (s, 1H), 7.90 (s, 1H), 7.86-7.75 (m, 1H), 7.44 (d, J = 1.9 Hz, 1H), 7.22-7.14 (m, 1H), 6.57 (s, 1H), 6.57- 6.45 (m, 1H), 6.36 (d, J = 8.5Hz, 1H), 5.18-5.12 (m, 1H), 4.42-4.30 (m, 2H), 4.28-4.13 (m, 2H), 3.82-3.68 (m, 2H), 3.55 (s, 3H), 2.33 (s, 3H), 2.04- 1.93 (m, 1H), 1.53 (d, J = 6.5 Hz, 3H), 1.04-0.87 (m, 4H). |
| 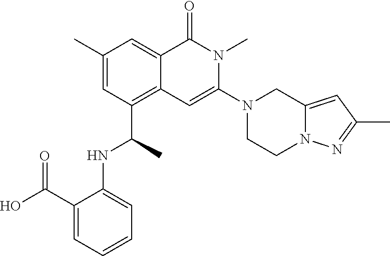 [SMILES: Cc1cc([C@@H](C)Nc2ccccc2C(=O)O)c2cc(N3CCn4nc(C)cc4C3)n(C)c(=O)c2c1] C₂₇H₂₉N₅O₃ | 446 | | ES− 470.4 [M − H] | ¹H NMR (400 MHz, Methanol-d₄) δ 8.01 (s, 1H), 7.92-7.90 (m, 1H), 7.57 (d, J = 1.7 Hz, 1H), 7.15-7.12 (m, 1H), 6.64 (s, 1H), 6.54-6.52 (m, 1H), 6.37 (d, J = 8.6 Hz, 1H), 6.00 (s, 1H), 5.0.7- 5.05 (m, 1H), 4.87-4.23 (m, 5H), 3.68 (s, 4H), 2.41 (s, 3H), 2.26 (s, 3H), 1.61 (d, J = 6.6 Hz, 3H). |
| 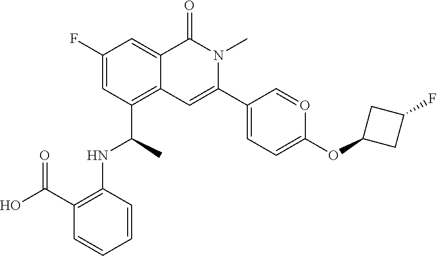 C₂₈H₂₅F₂N₃O₄ | 447 | | ES− 504.4 [M − H] | ¹H NMR (400 MHz, DMSO-d₆) δ 9.00 (s, 1H), 8.42 (d, J = 2.5 Hz, 1H), 8.02-8.00 (m, 1H), 7.85- 7.78 (m, 2H), 7.45- 7.43 (m, 1H), 7.08-7.04 (m, 1H), 7.03-6.95 (m, 2H), 6.50-6.46 (m, 1H), 6.25 (d, J = 8.4 Hz, 1H), 5.46-5.43 (m, 2H), 5.30- 5.26 (m, 1H), 3.37 (s, 3H), 2.74-2.65 (m, 2H), 2.61-2.50 (m, 2H), 1.51 (d, J = 6.5 Hz, 3H). |
| 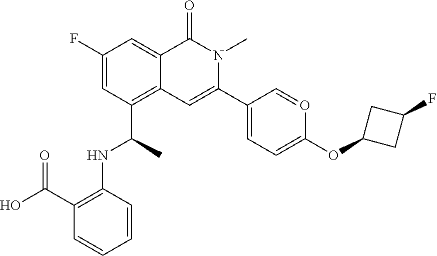 C₂₈H₂₅F₂N₃O₄ | 448 | | ES+ 506.0 [M + H] | ¹H NMR (400 MHz, DMSO-d₆) δ 12.81 (s, 1H), 8.41 (d, J = 2.5 Hz, 2H), 8.03-8.00 (m, 1H), 7.84- 7.81 (m, 2H), 7.44- 7.41 (m, 1H), 7.20-7.16 (m, 1H), 7.03-6.96 (m, 2H), 6.57-6.53 (m, 1H), 6.31 (d, J = 8.5 Hz, 1H), 5.32-5.30 (m, 1H), 5.02- 5.01 (m, 1H), 4.87- 4.84 (m, 1H), 3.37 (s, 3H), 3.06-3.00 (m, 2H), 2.34- 2.27 (m, 2H), 1.53 (d, J = 6.5 Hz, 3H). |
| 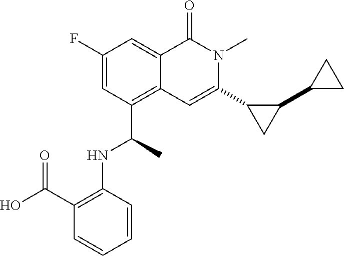 C₂₅H₂₅FN₂O₃ | 449 | trans cyclopropyl diastereomer 1 | ES− 419.2 [M − H] | ¹H NMR (400 MHz, DMSO-d₆) δ 12.76 (s, 1H), 8.37 (d, J = 6.0 Hz, 1H), 7.86-7.79 (m, 1H), 7.78- 7.72 (m, 1H), 7.43- 7.36 (m, 1H), 7.23-7.16 (m, 1H), 6.64 (s, 1H), 6.59- 6.51 (m, 1H), 6.33 (d, J = 8.4 Hz, 1H), 5.31-5.23 (m, 1H), 3.71 (s, 3H), 1.91- 1.89 (m, 1H), 1.52 (d, J = 6.6 Hz, 3H), 1.12-0.80 (m, 4H), 0.52-0.19 (m, 4H). |
| 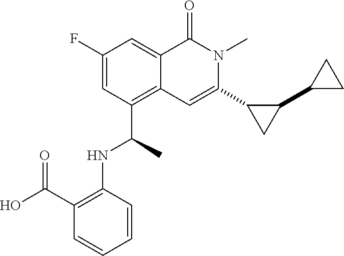 C₂₅H₂₅FN₂O₃ | 450 | trans cyclopropyl diastereomer 2 | ES− 419.2 [M − H] | ¹H NMR (400 MHz, DMSO-d₆) δ 12.80 (s, 1H), 8.36 (d, J = 6.0 Hz, 1H), 7.85-7.79 (m, 1H), 7.78- 7.72 (m, 1H), 7.42- 7.35 (m, 1H), 7.23-7.15 (m, 1H), 6.63 (s, 1H), 6.59- 6.52 (m, 1H), 6.32 (d, J = 8.4 Hz, 1H), 5.32-5.25 (m, 1H), 3.71 (s, 3H), 1.93- 1.85 (m, 1H), 1.52 (d, J = 6.5 Hz, 3H), 1.15-0.84 (m, 4H), 0.55-0.18 (m, 4H). |
| 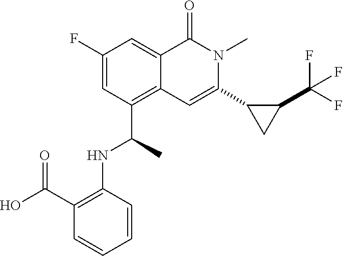 C₂₃H₂₀F₄N₂O₃ | 451 | trans cyclopropyl diastereomer 1 | ES− 447.2 [M − H] | ¹H NMR (400 MHz, DMSO-d₆) δ 8.55 (s, 1H), 7.83-7.74 (m, 2H), 7.41- 7.38 (m, 1H), 7.17- 7.13 (m, 1H), 6.83 (s, 1H), 6.55-6.52 (m, 1H), 6.31- 6.29 (m, 1H), 5.39- 5.38 (m, 1H), 3.68 (s, 3H), 2.70-2.67 (m, 2H), 1.52 (d, J = 6.6 Hz, 3H), 1.45- 1.42 (m, 2H). |
| 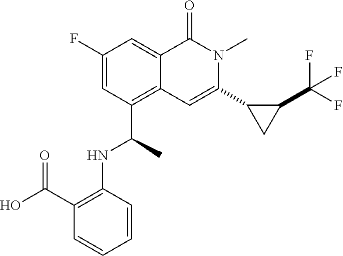 C₂₃H₂₀F₄N₂O₃ | 452 | trans cyclopropyl diastereomer 2 | ES− 447.2 [M − H] | ¹H NMR (400 MHz, DMSO-d₆) δ 8.45 (s, 1H), 7.84-7.75 (m, 1H), 7.77 (d, J = 9.1, 2.8 Hz, 1H), 7.42-7.39 (m, 1H), 7.18- 7.15 (m, 1H), 6.80 (s, 1H), 6.56-6.52 (m, 1H), 6.31-6.29 (m, 1H), 5.37- 5.36 (m, 1H), 3.68 (s, 3H), 2.71-2.66 (m, 2H), 1.69-1.67 (m, 1H), 1.53 (d, J = 6.5 Hz, 3H), 1.43- 1.42 (m, 1H). |
| 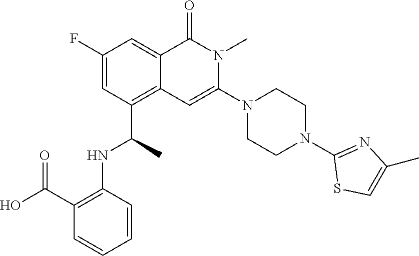 [SMILES: Cc1csc(N2CCN(c3cc4c([C@@H](C)Nc5ccccc5C(=O)O)cc(F)cc4c(=O)n3C)CC2)n1] C₂₇H₂₈FN₅O₃S | 453 | | ES+ 522.4 [M + H] | ¹H NMR (400 MHz, DMSO-d₆) δ 12.85 (s, 1H), 8.57 (s, 1H), 7.82-7.81 (m, 1H), 7.73-7.71 (m, 1H), 7.40-7.39 (m, 1H), 7.17-7.15 (m, 1H), 6.57- 6.48 (m, 2H), 6.45 (d, J = 1.3 Hz, 1H), 6.34 (d, J = 8.4 Hz, 1H), 5.25 (s, 1H), 3.76 (s, 5H), 3.55 (s, 2H), 3.07 (s, 4H), 2.18 (d, J = 1.1 Hz, 3H), 1.56 (d, J = 6.6 Hz, 3H). |
| 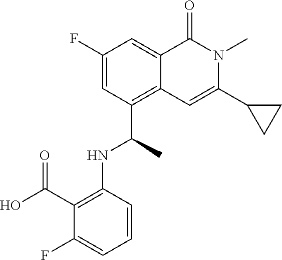 [SMILES: C[C@@H](Nc1cccc(F)c1C(=O)O)c1cc(F)cc2c(=O)n(C)c(C3CC3)cc12] C₂₂H₂₀F₂N₂O₃ | 454 | | ES− 397.2 [M − H] | ¹H NMR (400 MHz, Methanol-d₄) δ 7.83- 7.84 (m, 1H), 7.49-7.48 (m, 1H), 7.01-7.03 (m, 1H), 6.81 (s, 1H), 6.32- 6.30 (m, 1H), 6.04 (d, J = 8.5 Hz, 1H), 5.17-5.15 (m, 1H), 3.84 (s, 3H), 2.09- 2.06 (m, 1H), 1.61 (d, J = 6.6 Hz, 3H), 1.19-1.07 (m, 2H), 0.95-0.85 (m, 2H). |
| 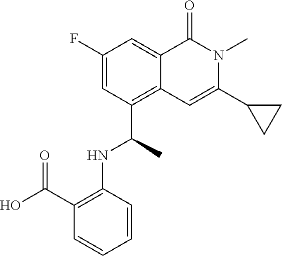 [SMILES: C[C@@H](Nc1ccccc1C(=O)O)c1cc(F)cc2c(=O)n(C)c(C3CC3)cc12] C₂₂H₂₁FN₂O₃ | 455 | | ES− 379.1 [M − H] | ¹H NMR (400 MHz, Methanol-d₄) δ 7.93-7.90 (m, 1H), 7.83-7.80 (m, 1H), 7.46-7.43 (m, 1H), 7.13-7.10 (m, 1H), 6.83 (s, 1H), 6.60-6.52 (m, 1H), 6.28 (d, J = 8.4 Hz, 1H), 5.19-5.13 (m, 1H), 3.84 (s, 3H), 2.08- 2.02 (m, 1H), 1.63 (d, J = 6.7 Hz, 3H), 1.19-1.06 (m, 2H), 0.93-0.84 (m, 2H). |
| 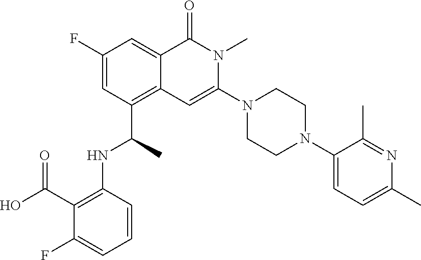 [SMILES: Cc1ccc(N2CCN(c3cc4c([C@@H](C)Nc5cccc(F)c5C(=O)O)cc(F)cc4c(=O)n3C)CC2)c(C)n1] C₃₀H₃₁F₂N₅O₃ | 456 | | ES+ 548.3 [M + H] | ¹H NMR (400 MHz, DMSO-d₆) δ 8.19 (s, 1H), 7.76-7.69 (m, 1H), 7.41 (d, J = 8.5 Hz, 2H), 7.06 (d, J = 8.1 Hz, 1H), 6.52 (d, J = 10.3 Hz, 2H), 6.30 (s, 1H), 6.10 (s, 1H), 5.24 (s, 1H), 3.57 (d, J = 1.2 Hz, 3H), 3.12 (d, J = 63.9 Hz, 8H), 2.46 (s, 3H), 2.38 (s, 3H), 1.54 (d, J = 6.4 Hz, 3H). |
| 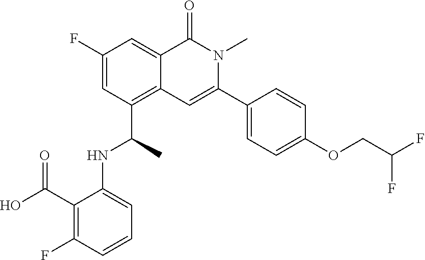 [SMILES: C[C@@H](Nc1cccc(F)c1C(=O)O)c1cc(F)cc2c(=O)n(C)c(-c3ccc(OCC(F)F)cc3)cc12] C₂₆H₂₁F₄N₃O₄ | 457 | | ES− 514.0 [M − H] | ¹H NMR (400 MHz, DMSO-d₆) δ 8.45 (d, J = 2.4 Hz, 1H), 8.21 (s, 1H), 8.11-8.04 (m, 1H), 7.86- 7.78 (m, 1H), 7.52- 7.44 (m, 1H), 7.10 (d, J = 8.6 Hz, 1H), 7.05-6.95 (m, 1H), 6.96 (s, 1H), 6.34- 6.23 (m, 2H), 6.04 (d, J = 8.4 Hz, 1H), 5.24 (d, J = 6.9 Hz, 1H), 4.73-4.60 (m, 2H), 3.36 (s, 3H), 1.49 (d, J = 6.5 Hz, 3H). |
| 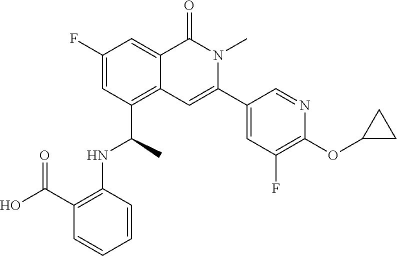 [SMILES: C[C@@H](Nc1ccccc1C(=O)O)c1cc(F)cc2c(=O)n(C)c(-c3cnc(OC4CC4)c(F)c3)cc12] C₂₇H₂₃F₂N₃O₄ | 458 | | ES− 490.3 [M − H] | ¹H NMR (400 MHz, DMSO-d₆) δ 8.72 (s, 1H), 8.31 (d, J = 2.0 Hz, 1H), 8.14-8.06 (m, 1H), 7.87- 7.79 (m, 2H), 7.47-7.41 (m, 1H), 7.16-7.08 (m, 1H), 7.04 (s, 1H), 6.56- 6.48 (m, 1H), 6.27 (d, J = 8.4 Hz, 1H), 5.30 (d, J = 7.0 Hz, 1H), 4.49-4.41 (m, 1H), 3.40 (s, 3H), 1.52 (d, J = 6.5 Hz, 3H), 0.88-0.76 (m, 4H). |
| 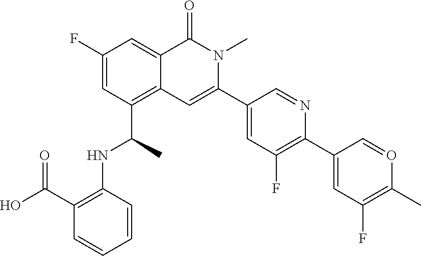 C₃₀H₂₃F₃N₄O₃ | 459 | | ES+ 545.3 [M + H] | ¹H NMR (300 MHz, DMSO-d₆) δ 8.92 (d, J = 17.6 Hz, 2H), 8.53 (s,1H),8.47 (s,1H),8.35 (d, J = 12.1 Hz, 1H), 8.19 (d, J = 10.6 Hz, 1H), 7.91- 7.79 (m, 1H), 7.48 (s, 1H), 7.16 (s, 1H), 6.56 (s, 1H), 6.53(s,1H) ,6.32 (d, J = 8.5 Hz, 1H), 5.36 (d, J = 8.5 Hz, 1H), 3.46 (s, 3H), 2.57 (d, J = 2.9 Hz, 3H), 1.55 (d, J = 6.4 Hz, 3H). |
| 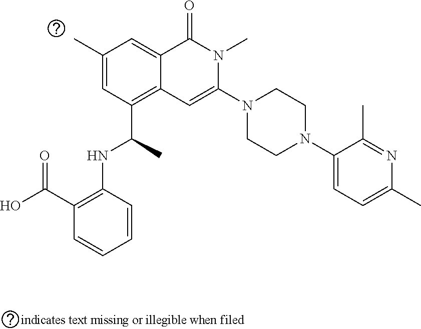 C₃₀H₃₂FN₅O₃ | 460 | | ES+ 530.1 [M + H] | ¹H NMR (400 MHz, DMSO-d₆) δ 12.75 (s, 1H), 8.41 (s, 1H), 7.85-7.78 (m, 1H), 7.76-7.69 (m, 1H), 7.44-7.34 (m, 2H), 7.25- 7.16 (m, 1H), 7.06 (d, J = 8.1 Hz, 1H), 6.60-6.50 (m, 2H), 6.37 (d, J = 8.4 Hz, 1H), 5.33-5.25 (m, 1H), 3.57 (s, 3H), 3.11 (d, J = 62.2 Hz, 8H), 2.46 (s, 3H), 2.38 (s, 3H), 1.57 (d, J = 6.6 Hz, 3H). |
| 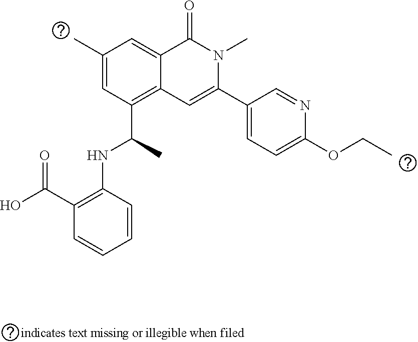 [SMILES: CCOc1ccc(-c2cc3c([C@@H](C)Nc4ccccc4C(=O)O)cc(C)cc3c(=O)n2C)cn1] C₂₆H₂₁F₄N₃O₄ | 461 | | ES− 514.2 [M − H] | ¹H NMR (400 MHz, DMSO-d₆) δ 12.82 (s, 1H), 8.48 (d, J = 2.4 Hz, 1H), 8.39 (d, J = 6.0 Hz, 1H), 8.14-8.09 (m, 1H), 7.87- 7.80 (m, 2H), 7.47-7.41 (m, 1H), 7.22-7.14 (m, 2H), 6.99 (s, 1H), 6.58- 6.52 (m, 1H), 6.32 (d, J = 8.5 Hz, 1H), 5.36-5.28 (m, 1H), 5.14-5.05 (m, 2H), 3.37 (s, 3H), 1.54 (d, J = 6.5 Hz, 3H). |
| 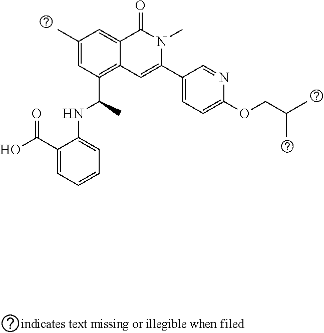 C₂₆H₂₂F₃N₃O₄ | 462 | | ES+ 498.3 [M + H] | ¹H NMR (400 MHz, DMSO-d₆) δ 8.45 (d, J = 2.4 Hz, 1H), 8.43-8.37 (m, 1H), 8.10-8.07 (m, 1H), 7.88-7.83 (m, 2H), 7.47-7.44 (m, 1H), 7.22- 7.14 (m, 1H), 7.10 (d, J = 8.6 Hz, 1H), 6.98 (s, 1H), 6.62-6.51 (m, 1H), 6.49- 6.45 (m, 1H), 6.35-6.28 (m, 1H), 5.31 (s, 1H), 4.75- 4.67 (m, 2H), 3.37 (s, 3H), 1.54 (d, J = 6.5 Hz, 3H) |
| 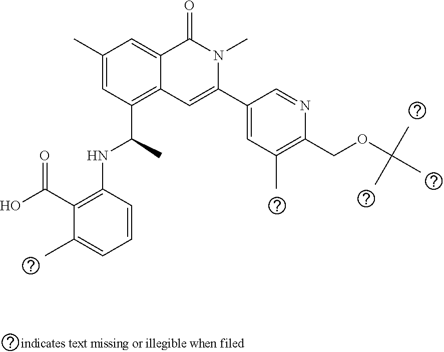 C₂₇H₂₂F₅N₃O₄ | 463 | | ES+ 548.4 [M + H] | ¹H NMR (400 MHz, DMSO-d₆) δ 13.30 (s, 1H), 8.77 (d, J = 1.6 Hz, 1H), 8.27-8.23 (m, 1H), 8.19 (s, 1H), 8.02 (s, 1H), 7.52 (d, J = 1.9 Hz, 1H), 7.06 (d, J = 14.1 Hz, 2H), 6.35-6.25 (m, 1H), 6.12 (d, J = 8.6 Hz, 1H), 5.37 (d, J = 1.9 Hz, 2H), 5.23-5.22 (m, 1H), 3.38 (s, 3H), 2.40 (s, 3H), 1.49 (d, J = 6.5 Hz, 3H). |
| 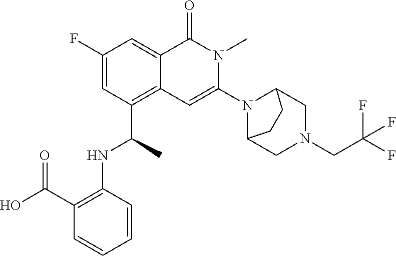 [SMILES: C[C@@H](Nc1ccccc1C(=O)O)c1cc(F)cc2c(=O)n(C)c(N3C4CCC3CN(CC(F)(F)F)C4)cc12] C₂₇H₂₈F₄N₄O₃ | 464 | | ES+ 533.4 [M + H] | ¹H NMR (400 MHz, DMSO-d₆) δ 8.64 (s, 1H), 7.82-7.81 (m, 1H), 7.68- 7.67 (m, 1H), 7.38- 7.37 (m, 1H), 7.16-7.14 (m, 1H), 6.53-6.51 (m, 1H), 6.36 (d, J = 8.4 Hz, 1H), 6.18 (s, 1H), 5.17- 5.15 (m, 1H), 3.92 (s, 1H), 3.91 (s, 1H), 3.86 (s, 3H), 3.27-3.24 (m, 2H), 2.90- 2.75 (m, 4H), 1.98- 1.85 (m, 4H), 1.55 (d, J = 6.6 Hz, 3H). |
| 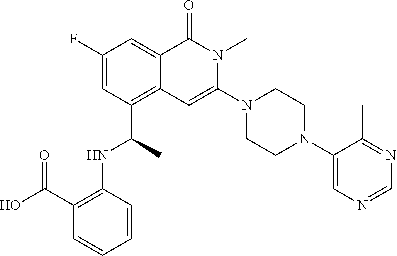 [SMILES: Cc1ncncc1N1CCN(c2cc3c([C@@H](C)Nc4ccccc4C(=O)O)cc(F)cc3c(=O)n2C)CC1] C₂₈H₂₉FN₆O₃ | 465 | | ES+ 517.4 [M + H] | ¹H NMR (400 MHz, Methanol-d₄) δ 8.75 (s, 1H), 8.50 (s, 1H), 7.98- 7.91 (m, 1H), 7.87-7.77 (m, 1H), 7.52-7.44 (m, 1H), 7.24-7.16 (m, 1H), 6.65-6.54 (m, 2H), 6.41 (d, J = 8.5 Hz, 1H), 5.25- 5.17 (m, 1H), 3.72 (s, 3H), 3.32-3.03 (m, 8H), 2.61 (s, 3H), 1.69 (d, J = 6.6 Hz, 3H). |
| 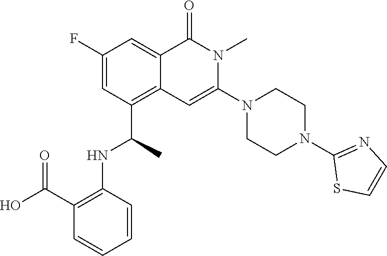 [SMILES: C[C@@H](Nc1ccccc1C(=O)O)c1cc(F)cc2c(=O)n(C)c(N3CCN(c4nccs4)CC3)cc12] C₂₆H₂₆FN₅O₃S | 466 | | ES− 506.3 [M − H] | ¹H NMR (400 MHz, DMSO-d₆) δ 12.84 (s, 1H), 8.42 (s, 1H), 7.82-7.80 (m, 1H), 7.77-7.70 (m, 1H), 7.41-7.35 (m, 1H), 7.25-7.16 (m, 2H), 6.92 (d, J = 3.6 Hz, 1H), 6.59- 6.51 (m, 1H), 6.50 (s, 1H), 6.35 (d, J = 8.5 Hz, 1H), 5.27-5.24 (m, 1H), 4.15- 3.57 (m, 7H), 3.32- 3.04 (m, 4H), 1.57 (d, J = 6.5 Hz, 3H). |
| 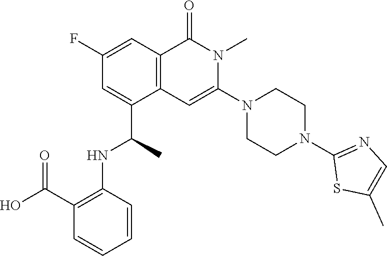 [SMILES: Cc1cnc(N2CCN(c3cc4c([C@@H](C)Nc5ccccc5C(=O)O)cc(F)cc4c(=O)n3C)CC2)s1] C₂₇H₂₈FN₅O₃S | 467 | | ES+ 522.4 [M + H] | ¹H NMR (400 MHz, Methanol-d₄) δ 7.97- 7.88 (m, 1H), 7.85-7.76 (m, 1H), 7.52-7.41 (m, 1H), 7.23-7.12 (m, 1H), 6.89-6.81 (m, 1H), 6.62- 6.51 (m, 2H), 6.37 (d, J = 8.5 Hz, 1H), 5.18- 5.15(m, 1H), 3.77 (s, 2H), 3.69 (s, 3H), 3.40-3.35 (m, 2H), 3.30-2.90 (m, 4H), 2.33 (s, 3H), 1.66 (d, J = 6.6 Hz, 3H). |
| 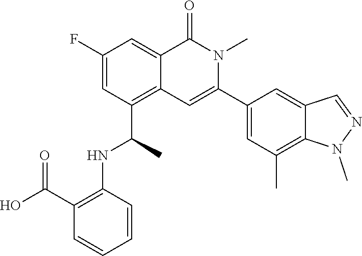 C₂₇H₂₂F₂N₄O₃ | 468 | | ES+ 489.0 [M + H] | ¹H NMR (400 MHz, DMSO-d₆) δ 8.60 (s, 1H), 8.25 (d, J = 2.3 Hz, 1H), 7.89-7.78 (m, 3H), 7.60- 7.53 (m, 1H), 7.48- 7.41 (m, 1H), 7.19-7.10 (m, 1H), 6.96 (s, 1H), 6.57- 6.50 (m, 1H), 6.29 (d, J = 8.5 Hz, 1H), 5.27-5.17 (m, 1H), 4.24 (s, 3H), 3.37 (s, 3H), 1.53 (d, J = 6.6 Hz, 3H). |
| 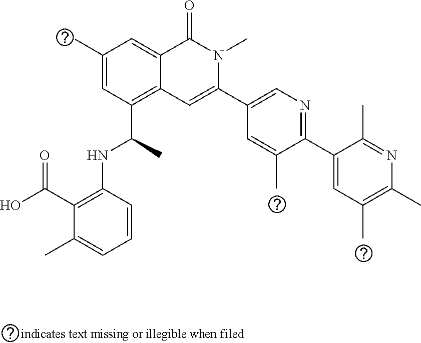 C₃₁H₂₄F₄N₄O₃ | 469 | | ES+ 577.2 [M + H] | ¹H NMR (400 MHz, DMSO-d₆) δ 13.28 (s, 1H), 8.85 (d, J = 1.8 Hz, 1H), 8.32-8.30 (m, 1H), 8.09 (s, 1H), 7.90-7.88 (m, 1H), 7.74 (d, J = 9.8 Hz, 1H), 7.50-7.49 (m, 1H), 7.17-7.09 (m, 2H), 6.39- 6.36 (m, 1H), 6.12 (d, J = 8.6 Hz, 1H), 5.34 (m, 1H), 3.46 (s, 3H), 2.50 (s, 3H), 2.42 (s, 3H), 1.54 (d, J = 6.5 Hz, 3H). |
| 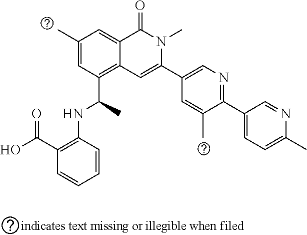 C₂₉H₂₁F₃N₄O₃ | 470 | | ES+ 531.3 [M + H] | ¹H NMR (400 MHz, DMSO-d₆) δ 9.07 (s, 1H), 8.91 (s, 1H), 8.77 (d, J = 2.8 Hz, 1H), 8.40-8.32 (m, 1H), 8.27 (d, J = 9.6 Hz, 1H), 7.89-7.80 (m, 8.6 Hz, 2H), 7.47 (d, J = 8.0 Hz, 1H), 7.17-7.11 (m, 2H), 7.12 (s, 1H), 6.53 (d, J = 7.8 Hz, 1H), 6.29 (d, J = 8.5 Hz, 1H), 5.33 (s, 1H), 3.46 (s, 3H), 1.54 (d, J = 6.5 Hz, 3H). |
| 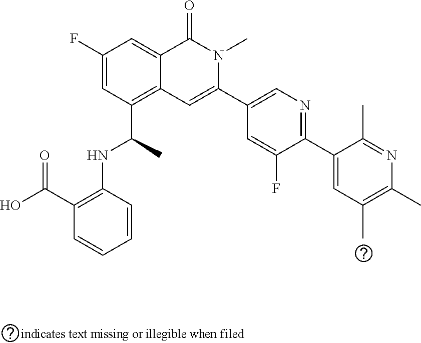 C₃₁H₂₅F₃N₄O₃ | 471 | | ES+ 559.3 [M + H] | ¹H NMR (400 MHz, DMSO-d₆) δ 12.84 (s, 1H), 8.85 (d, J = 1.6 Hz, 1H), 8.43 (s, 1H), 8.33-8.31 (m, 1H), 7.90-7.85 (m, 2H), 7.74 (d, J = 9.8 Hz, 1H), 7.49-7.46 (m, 1H), 7.23-7.13 (m, 2H), 6.59- 6.56 (m, 1H), 6.33 (d, J = 8.5 Hz, 1H), 5.36 (s, 1H), 3.31 (s, 3H), 2.50 (s, 3H), 2.42 (s, 3H), 1.56 (d, J = 6.5 Hz, 3H). |
| 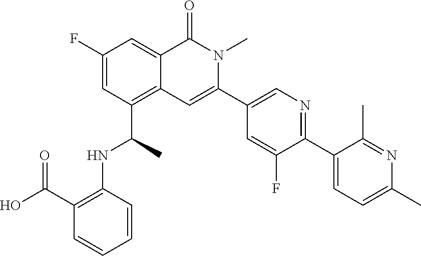 [SMILES: Cc1ccc(-c2ncc(-c3cc4c([C@@H](C)Nc5ccccc5C(=O)O)cc(F)cc4c(=O)n3C)cc2F)c(C)n1] C₃₁H₂₆F₂N₄O₃ | 472 | | ES+ 541.3 [M + H] | ¹H NMR (400 MHz, DMSO-d₆) 812.8 (s, 1H), 8.83 (d, J = 1.7 Hz, 1H), 8.54 (s, 1H), 8.29-8.24 (m, 1H), 7.89-7.80 (m, 2H), 7.74 (d, J = 7.8 Hz, 1H), 7.48-7.43 (m, 1H), 7.26 (d, J = 7.9 Hz, 1H), 7.20-7.11 (m, 2H), 6.57-6.51 (m, 1H), 6.32 (d, J = 8.5 Hz, 1H), 5.36 (s, 1H), 3.46 (s, 3H), 2.53 (s, 3H), 2.43 (s, 3H), 1.55 (d, J = 6.6 Hz, 3H) |
| 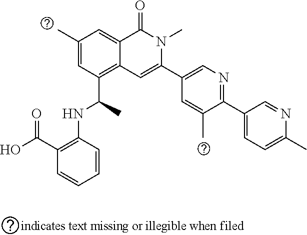 C₃₀H₂₄F₂N₄O₃ | 473 | | ES+ 527.2 [M + H] | ¹H NMR (400 MHz, DMSO-d₆) δ 12.84 (s, 1H), 9.06 (s, 1H), 8.86 (s, 1H), 8.43 (s, 1H), 8.32-8.25 (m, 2H), 7.90-7.79 (m, 2H), 7.51-7.42 (m, 2H), 7.23-7.12 (m, 2H), 6.59- 6.52 (m, 1H), 6.33 (d, J = 8.5 Hz, 1H), 5.35 (s, 1H), 3.46 (s, 3H), 2.58 (s, 3H), 1.55 (d, J = 6.5 Hz, 3H). |
| 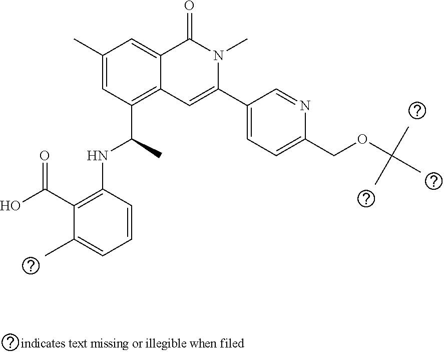 [SMILES: Cc1cc([C@@H](C)Nc2cccc(C)c2C(=O)O)c2cc(-c3ccc(COC(C)(C)C)nc3)n(C)c(=O)c2c1] C₂₇H₂₃F₄N₃O₄ | 474 | | ES+ 528.1 [M − H] | ¹H NMR (400 MHz, Methanol-d₄) δ 8.79 (d, J = 2.2 Hz, 1H), 8.15-8.08 (m, 2H), 7.72 (d, J = 8.1 Hz, 1H), 7.67 (d, J = 1.9 Hz, 1H), 7.01 (s, 1H), 6.99- 6.90 (m, 1H), 6.30- 6.21 (m, 1H), 6.06 (d, J = 8.4 Hz, 1H), 5.28 (s, 2H), 5.16-5.06 (m, 1H), 3.47 (s, 3H), 2.44 (s, 3H), 1.58 (d, J = 6.6 Hz, 3H). |
| 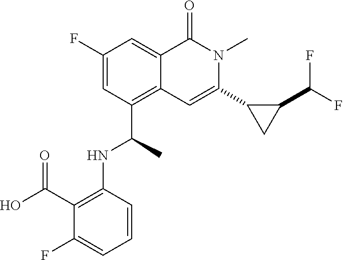 C₂₃H₂₀F₄N₂O₃ | 475 | trans cyclopropyl diastereomer 1 | ES+ 449.3 [M + H] | ¹H NMR (400 MHz, Methanol-d₄) δ 7.89- 7.82 (m, 1H), 7.52-7.44 (m, 1H), 7.14-7.05 (m, 1H), 6.85 (s, 1H), 6.37- 6.28 (m, 1H), 6.13-6.06 (m, 1H), 5.94 (d, J = 4.6 Hz, 1H), 5.25-5.16 (m, 1H), 3.82 (s, 3H), 2.49- 2.40 (m, 1H), 1.89-1.85 (m, 1H), 1.63 (d, J = 6.9 Hz, 3H), 1.42-1.34 (m, 2H). |
| 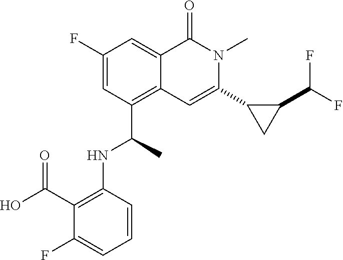 C₂₃H₂₀F₄N₂O₃ | 476 | trans cyclopropyl diastereomer 2 | ES+ 449.3 [M + H] | ¹H NMR (400 MHz, Methanol-d₄) δ 7.90-7.82 (m, 1H), 7.51-7.45(m, 1H), 7.14-7.04 (m, 1H), 6.84 (s, 1H), 6.37-6.28 (m, 1H), 6.11-6.09 (m, 1H), 5.95 (d, J = 4.5 Hz, 1H), 5.26-5.16 (m, 1H), 3.82 (s, 3H), 2.49-2.39 (m, 1H), 1.93-1.81 (m, 1H), 1.63 (d, J = 6.6 Hz, 3H), 1.44-1.33 (m, 2H). |
| 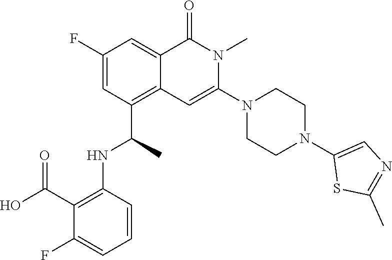 [SMILES: Cc1ncc(N2CCN(c3cc4c([C@@H](C)Nc5cccc(F)c5C(=O)O)cc(F)cc4c(=O)n3C)CC2)s1] C₂₇H₂₇F₂N₅O₃S | 477 | | ES+ 540.1 [M + H] | ¹H NMR (400 MHz, DMSO-d₆) δ 8.14 (s, 1H), 7.72-7.62 (m, 1H), 7.42- 7.40 (m, 1H), 7.10- 7.00 (m, 1H), 6.88 (s, 1H), 6.47 (s, 1H), 6.32-6.25 (m, 1H), 6.12 (d, J = 8.5 Hz, 1H), 5.21-5.11 (m, 1H), 3.54 (s, 3H), 3.44- 2.84 (m, 11H), 1.54 (d, J = 6.5 Hz, 3H). |
| 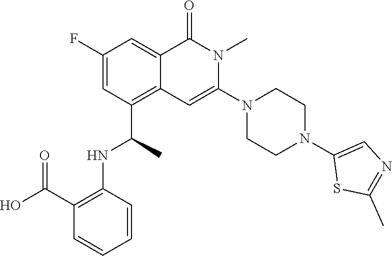 [SMILES: Cc1ncc(N2CCN(c3cc4c([C@@H](C)Nc5ccccc5C(=O)O)cc(F)cc4c(=O)n3C)CC2)s1] C₂₇H₂₈FN₅O₃S | 478 | | ES+ 522.0 [M + H] | ¹H NMR (400 MHz, DMSO-d₆) δ 8.52 (s, 1H), 7.82-7.78 (m, 1H), 7.72- 7.69 (m, 1H), 7.39- 7.30 (m, 1H), 7.18-7.08 (m, 1H), 6.88 (s, 1H), 6.59- 6.45 (m, 2H), 6.34 (d, J = 8.5 Hz, 1H), 5.24-5.14 (m, 1H), 3.54 (s, 3H), 3.49- 2.89 (m, 11H), 1.56 (d, J = 6.5 Hz, 3H). |
| 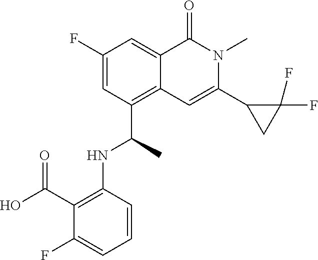 [SMILES: C[C@@H](Nc1cccc(F)c1C(=O)O)c1cc(F)cc2c(=O)n(C)c(C3CC3(F)F)cc12] C₂₂H₁₈F₄N₂O₃ | 479 | diastereomer 1 | ES+ 435.2 [M + H] | ¹H NMR (400 MHz, Methanol-d₄) δ 7.91- 7.82 (m, 1H), 7.53-7.45 (m, 1H), 7.13-7.02 (m, 1H), 6.98 (d, J = 1.4 Hz, 1H), 6.36-6.26 (m, 1H), 6.09 (d, J = 8.5 Hz, 1H), 5.24-5.13 (m, 1H), 3.73 (s, 3H), 3.23-3.07 (m, 1H), 2.21-1.97 (m, 2H), 1.61 (d, J = 6.7 Hz, 3H). |
| 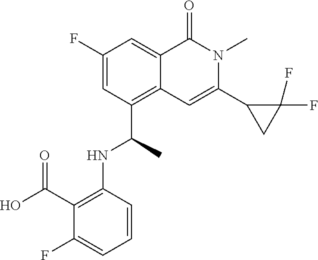 [SMILES: C[C@@H](Nc1cccc(F)c1C(=O)O)c1cc(F)cc2c(=O)n(C)c(C3CC3(F)F)cc12] C₂₂H₁₈F₄N₂O₃ | 480 | diastereomer 2 | ES+ 435.2 [M + H] | ¹H NMR (400 MHz, Methanol-d₄) δ 7.91- 7.82 (m, 1H), 7.52-7.43 (m, 1H), 7.16-7.03 (m, 1H), 6.98 (d, J = 1.4 Hz, 1H), 6.38-6.25 (m, 1H), 6.09 (d, J = 8.5 Hz, 1H), 5.32-5.15 (m, 1H), 3.74 (s, 3H), 3.21-3.04(m, 1H), 2.21-1.99 (m, 2H), 1.63 (d, J = 6.7 Hz, 3H). |
| 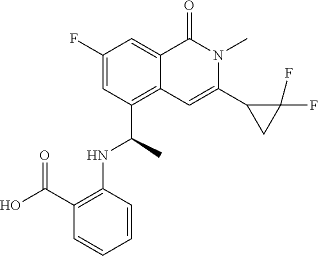 [SMILES: C[C@@H](Nc1ccccc1C(=O)O)c1cc(F)cc2c(=O)n(C)c(C3CC3(F)F)cc12] C₂₂H₁₉F₃N₂O₃ | 481 | diastereomer 1 | ES+ 417.2 [M + H] | ¹H NMR (400 MHz, Methanol-d₄) δ 7.97- 7.90 (m, 1H), 7.90-7.83 (m, 1H), 7.54-7.46 (m, 1H), 7.19-7.11 (m, 1H), 7.02 (d, J = 1.4 Hz, 1H), 6.63-6.51 (m, 1H), 6.32 (d, J = 8.4 Hz, 1H), 5.32- 5.11 (m, 1H), 3.74 (s, 3H), 3.24-3.05 (m, 1H), 2.24- 1.94 (m, 2H), 1.64 (d, J = 6.7 Hz, 3H). |
| 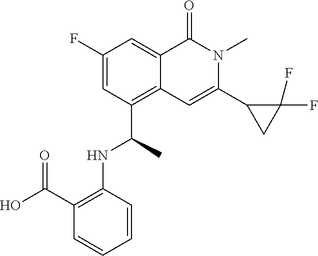 [SMILES: C[C@@H](Nc1ccccc1C(=O)O)c1cc(F)cc2c(=O)n(C)c(C3CC3(F)F)cc12] C₂₂H₁₉F₃N₂O₃ | 482 | diastereomer 2 | ES+ 417.2 [M + H] | ¹H NMR (400 MHz, Methanol-d₄) δ 7.98- 7.90 (m, 1H), 7.90-7.82 (m, 1H), 7.53-7.43 (m, 1H), 7.20-7.12 (m, 1H), 7.00 (d, J = 1.4 Hz, 1H), 6.62-6.54 (m, 1H), 6.32 (d, J = 8.4 Hz, 1H), 5.32- 5.21 (m, 1H), 3.74 (s, 3H), 3.19-3.07 (m, 1H), 2.17- 1.98 (m, 2H), 1.65 (d, J = 6.6 Hz, 3H). |
| 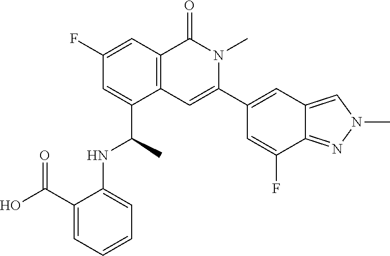 [SMILES: C[C@@H](Nc1ccccc1C(=O)O)c1cc(F)cc2c(=O)n(C)c(-c3cc(F)c4nn(C)cc4c3)cc12] C₂₇H₂₂F₂N₄O₃ | 483 | | ES+ 489.0 [M + H] | ¹H NMR (400 MHz, DMSO-d₆) δ 8.72 (s, 1H), 8.62 (d, J = 2.8 Hz, 1H), 7.87-7.77 (m, 3H), 7.37- 7.31 (m, 1H), 7.20 (s, 1H), 7.18-7.06 (m, 1H), 6.97 (s, 1H), 6.55-6.48 (m, 1H), 6.27 (d, J = 8.5 Hz, 1H), 5.27-5.17 (m, 1H), 4.25 (s, 3H), 3.38 (s, 3H), 1.53 (d, J = 6.6 Hz, 3H). |
| 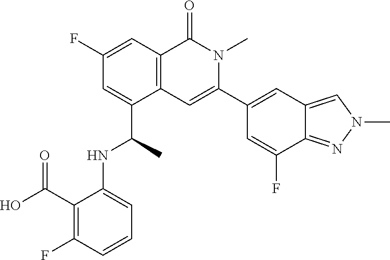 [SMILES: C[C@@H](Nc1cccc(F)c1C(=O)O)c1cc(F)cc2c(=O)n(C)c(-c3cc(F)c4nn(C)cc4c3)cc12] C₂₇H₂₁F₃N₄O₃ | 484 | | ES+ 507.2 [M + H] | ¹H NMR (400 MHz, DMSO-d₆) δ 8.61 (d, J = 2.8 Hz, 1H), 8.18 (s, 1H), 7.87-7.78 (m, 2H), 7.50- 7.44 (m, 1H), 7.37- 7.31 (m, 1H), 7.07-6.98 (m, 1H), 6.95 (s, 1H), 6.34- 6.25 (m, 1H), 6.05 (d, J = 8.5 Hz, 1H), 5.23-5.18 (m, 1H), 4.25 (s, 3H), 3.38 (s, 3H), 1.50 (d, J = 6.5 Hz, 3H). |
| 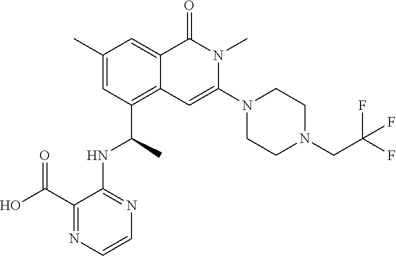 [SMILES: Cc1cc([C@@H](C)Nc2nccnc2C(=O)O)c2cc(N3CCN(CC(F)(F)F)CC3)n(C)c(=O)c2c1] C₂₄H₂₇F₃N₆O₃ | 485 | | ES+ 505.4 [M + H] | ¹H NMR (400 MHz, Methanol-d₄) δ 8.20 (s, 1H), 8.00 (s, 1H), 7.60 (s, 1H), 7.50 (s, 1H), 6.45 (s, 1H), 5.83-5.78 (m, 1H), 3.61 (s, 3H), 3.25-2.65 (m, 10 H), 2.49-2.31 (m, 3H), 1.78-1.62 (m, 3H). |
| 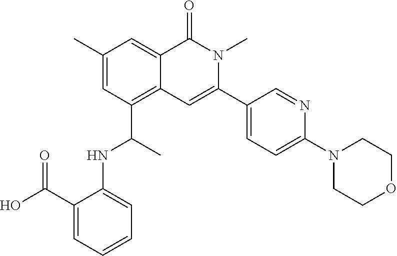 [SMILES: Cc1cc(C(C)Nc2ccccc2C(=O)O)c2cc(-c3ccc(N4CCOCC4)nc3)n(C)c(=O)c2c1] C₂₉H₃₀N₄O₄ | 486 | enantiomer 1 | ES− 497.1 [M − H] | ¹H NMR (400 MHz, Methanol-d₄) δ 8.29 (d, J = 8.0 Hz, 1H), 8.08 (s, 1H), 7.93-7.88 (m, 1H), 7.76- 7.71 (m, 1H), 7.61 (d, J = 4.0 Hz, 1H), 7.15-7.08 (m, 1H), 6.96 (s, 1H), 6.94 (s, 1H), 6.56-6.49 (m, 1H), 6.32 (d, J = 8.0 Hz, 1H), 5.19-5.11 (m, 1H), 3.86-3.80 (m, 4H), 3.65- 3.58 (m, 4H), 3.50 (s, 3H), 2.42 (s, 3H), 1.62 (d, J = 8.0 Hz, 3H). |
| 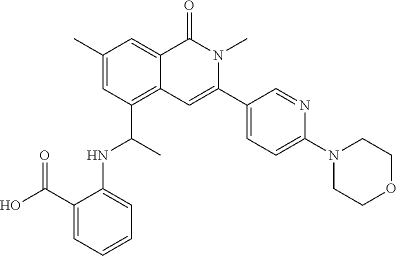 [SMILES: Cc1cc(C(C)Nc2ccccc2C(=O)O)c2cc(-c3ccc(N4CCOCC4)nc3)n(C)c(=O)c2c1] C₂₉H₃₀N₄O₄ | 487 | enantiomer 2 | ES− 497.1 [M − H] | ¹H NMR (400 MHz, Methanol-d₄) δ 8.29 (d, J = 8.0 Hz, 1H), 8.08 (s, 1H), 7.93-7.88 (m, 1H), 7.76- 7.71 (m, 1H), 7.61 (d, J = 4.0 Hz, 1H), 7.15-7.08 (m, 1H), 6.96 (s, 1H), 6.94 (s, 1H), 6.56-6.49 (m, 1H), 6.32 (d, J = 8.0 Hz, 1H), 5.19-5.11 (m, 1H), 3.86-3.80 (m, 4H), 3.65- 3.58 (m, 4H), 3.50 (s, 3H), 2.42 (s, 3H), 1.62 (d, J = 8.0 Hz, 3H). |
| 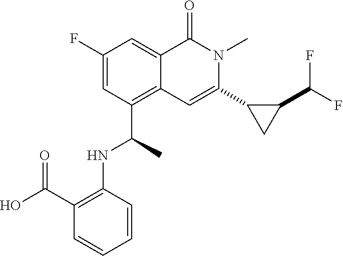 C₂₃H₂₁F₃N₂O₃ | 488 | trans cyclopropyl diastereomer 1 | ES+ 431.3 [M + H] | ¹H NMR (400 MHz, Methanol-d₄) δ 8.44 (s, 1H), 7.94 (d, J = 7.7 Hz, 1H), 7.86-7.83 (m, 1H), 7.49-7.46 (m, 1H), 7.16- 7.09 (m, 1H), 6.86 (s, 1H), 6.59-6.54 (m, 1H), 6.31 (d, J = 8.2 Hz, 1H), 5.95-5.81 (m, 1H), 5.22- 5.20 (m, 1H), 3.82 (s, 3H), 2.43-2.42 (m, 1H), 1.86- 1.85 (m, 1H), 1.64 (d, J = 6.5 Hz, 3H), 1.40-1.28 (m, 1H). |
| 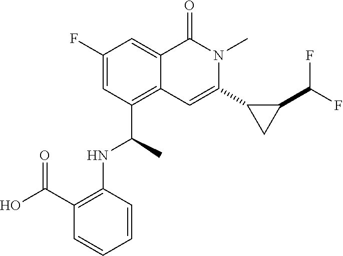 C₂₃H₂₁F₃N₂O₃ | 489 | trans cyclopropyl diastereomer 2 | ES+ 431.3 [M + H] | ¹H NMR (400 MHz, Methanol-d₄) δ 8.44 (s, 1H), 7.94 (d, J = 8.0 Hz, 1H), 7.85-7.82 (m, 1H), 7.49-7.46 (m, 1H), 7.17- 7.14 (m, 1H), 6.87 (s, 1H), 6.59-6.56 (m, 1H), 6.33 (d, J = 8.5 Hz, 1H), 5.94-5.79 (m, 1H), 5.22- 5.21 (m, 1H), 3.82 (s, 3H), 2.43-2.42 (m, 1H), 1.86- 1.85 (m, 1H), 1.65 (d, J = 6.4 Hz, 3H), 1.41-1.33 (m, 1H). |
| 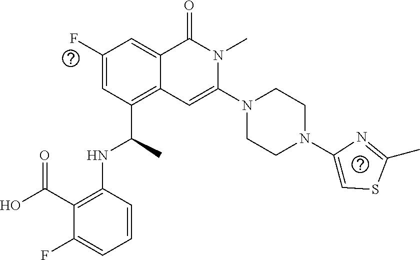 [SMILES: Cc1nc(N2CCN(c3cc4c([C@@H](C)Nc5cccc(F)c5C(=O)O)cc(F)cc4c(=O)n3C)CC2)cs1] C₂₇H₂₇F₂N₅O₃S | 490 | | ES− 538.2 [M − H] | ¹H NMR (400 MHz, DMSO-d₆) δ 8.26 (s, 1H), 7.72-7.71 (m, 1H), 7.43- 7.41 (m, 1H), 7.23- 6.95 (m, 1H), 6.46 (s, 1H), 6.19-6.18 (m, 1H), 6.15 (d, J = 1.4 Hz, 1H), 6.07 (d, J = 8.4 Hz, 1H), 5.19- 5.15 (m, 1H), 4.00-3.60 (m, 1H), 3.55 (s, 4H), 3.33- 2.80 (m, 6H), 2.59 (s, 3H), 1.52 (d, J = 6.5 Hz, 3H). |
| 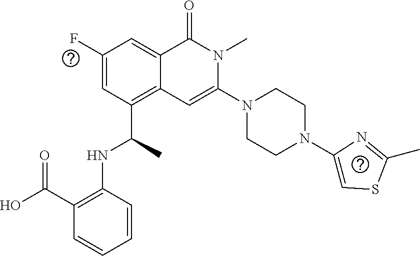 [SMILES: Cc1nc(N2CCN(c3cc4c([C@@H](C)Nc5ccccc5C(=O)O)cc(F)cc4c(=O)n3C)CC2)cs1] C₂₇H₂₈FN₅O₃S | 491 | | ES− 520.3 [M − H] | ¹H NMR (400 MHz, DMSO-d₆) δ 12.83 (s, 1H), 8.55 (s, 1H), 7.83-7.82 (m, 1H), 7.74-7.73 (m, 1H), 7.41-7.39 (m, 1H), 7.20-7.19 (m, 1H), 6.54- 6.52 (m, 1H), 6.48 (s, 1H), 6.36-6.34 (m, 1H), 6.15 (s, 1H), 5.24-5.20 (m, 1H), 3.81-3.60 (m, 1H), 3.55 (s, 4H), 3.33- 2.80 (m,6H), 2.59 (s, 3H), 1.57 (d, J = 6.6 Hz, 3H). |
| 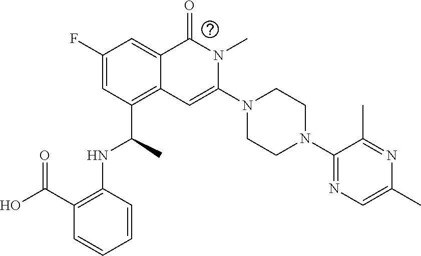 [SMILES: Cc1cnc(N2CCN(c3cc4c([C@@H](C)Nc5ccccc5C(=O)O)cc(F)cc4c(=O)n3C)CC2)c(C)n1] C₂₉H₃₁FN₆O₃ | 492 | | ES+ 531.3 [M + H] | ¹H NMR (400 MHz, Methanol-d₄) δ 8.04 (s, 1H), 7.98-7.88 (m, 1H), 7.86-7.75 (m, 1H), 7.51- 7.40 (m, 1H), 7.24- 7.11 (m, 1H), 6.62-6.52 (m, 2H), 6.39 (d, J = 8.5 Hz, 1H), 5.25-5.14 (m, 1H), 3.70 (s, 3H), 3.52 (s, 2H), 3.23-3.00 (m, 6H), 2.56 (s, 3H), 2.45 (s, 3H), 1.68 (d, J = 6.7 Hz, 3H). |
| 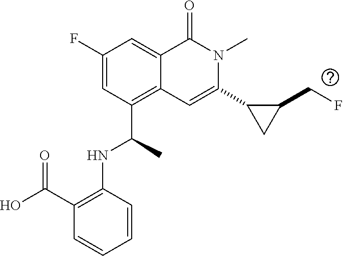 C₂₃H₂₂F₂N₂O₃ | 493 | trans cyclopropyl diastereomer 1 | ES+ 413.0 [M + H] | ¹H NMR (400 MHz, Methanol-d₄) δ 7.96- 7.89 (m, 1H), 7.85-7.79 (m, 1H), 7.48-7.43 (m, 1H), 7.16-7.13 (m, 1H), 6.85 (s, 1H), 6.61-6.53 (m, 1H), 6.35-6.30 (m, 1H), 5.22-5.17 (m, 1H), 4.75-4.59 (m, 1H), 4.45- 4.29 (m, 1H), 3.83 (s, 3H), 2.18-2.17 (m, 1H), 1.64-1.62 (m, 4H), 1.36- 1.26 (m, 1H), 1.17- 1.14 (m, 1H). |
| 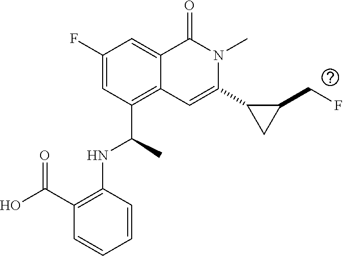 C₂₃H₂₂F₂N₂O₃ | 494 | trans cyclopropyl diastereomer 2 | ES+ 413.0 [M + H] | ¹H NMR (400 MHz, Methanol-d₄) δ 7.94- 7.92 (m, 1H), 7.86-7.81 (m, 1H), 7.46-7.44 (m, 1H), 7.15-7.13 (m, 1H), 6.83 (s, 1H), 6.58-6.55 (m, 1H), 6.31-6.30 (m, 1H), 5.21-5.20 (m, 1H), 4.76-4.72 (m, 1H), 4.63- 4.59 (m, 1H), 3.85 (s, 3H), 2.19-2.17 (m, 1H), 1.67-1.62 (m, 4H), 1.30- 1.29 (m, 1H), 1.13- 1.12 (m, 1H). |
| 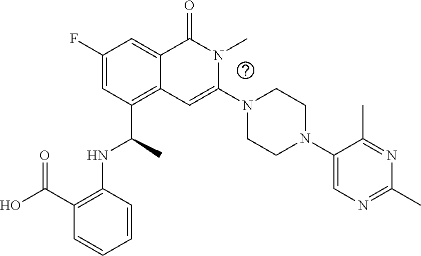 [SMILES: Cc1ncc(N2CCN(c3cc4c([C@@H](C)Nc5ccccc5C(=O)O)cc(F)cc4c(=O)n3C)CC2)c(C)n1] C₂₉H₃₁FN₆O₃ | 495 | | ES+ 531.4 [M + H] | ¹H NMR (400 MHz, Methanol-d₄) δ 8.39 (s, 1H), 7.95-7.93 (m, 1H), 7.91-7.79 ( m, 1H), 7.50- 7.47 (m, 1H), 7.19- 7.11 (m, 1H), 6.61 (d, J = 2.2 Hz, 1H), 6.56-6.54 (m, 1H), 6.37-6.36 (m, 1H), 5.19-5.16 (m, 1H), 3.71 (s, 3H), 3.33 (s, 2H), 3.30-3.00 (m, 6H), 2.63 (s, 3H), 2.58 (s, 3H), 1.68 (d, J = 6.6 Hz, 3H). |
| 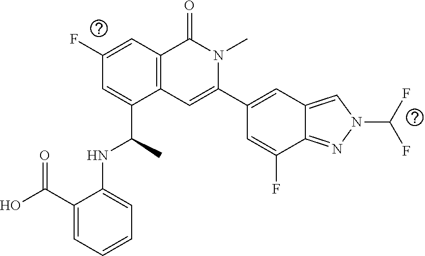 [SMILES: C[C@@H](Nc1ccccc1C(=O)O)c1cc(F)cc2c(=O)n(C)c(-c3cc(F)c4nn(C(F)F)cc4c3)cc12] C₂₇H₂₀F₄N₄O₃ | 496 | | ES+ 525.1 [M + H] | ¹H NMR (400 MHz, DMSO-d₆) δ 9.20 (s, 1H), 8.60-8.03 (m, 2H), 8.00- 7.73 (m, 3H), 7.60-7.53 (m, 1H), 7.50-7.40 (m, 1H), 7.20-7.13 (m, 1H), 7.04 (s, 1H), 6.56 (s, 1H), 6.33 (s, 1H), 5.31 (m, 1H), 3.4 (s, 3H), 1.65-1.49 (m, 3H) |
| 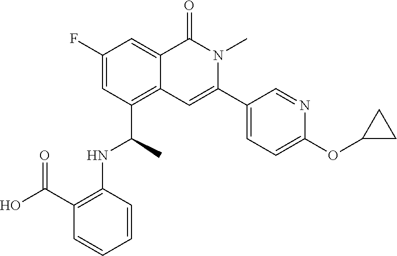 [SMILES: C[C@@H](Nc1ccccc1C(=O)O)c1cc(F)cc2c(=O)n(C)c(-c3ccc(OC4CC4)nc3)cc12] C₂₇H₂₄FN₃O₄ | 497 | | ES− 472.1 [M − H] | ¹H NMR (400 MHz, DMSO-d₆) δ 8.46 (d, J = 2.5 Hz, 1H), 8.43 (s, 1H), 8.05-7.98 (m, 1H), 7.87- 7.79 (m, 2H), 7.47- 7.39 (m, 1H), 7.22-7.13 (m, 1H), 7.06-6.96 (m, 2H), 6.59-6.51 (m, 1H), 6.31 (d, J = 8.5 Hz, 1H), 5.35-5.28 (m, 1H), 4.34- 4.25 (m, 1H), 3.38- 3.17 (m, 3H) , 1.53 (d, J = 6.6 Hz, 3H), 0.88-0.77 (m, 2H), 0.80-0.69 (m, 2H). |
| 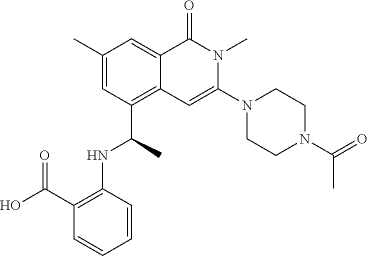 [SMILES: CC(=O)N1CCN(c2cc3c([C@@H](C)Nc4ccccc4C(=O)O)cc(C)cc3c(=O)n2C)CC1] C₂₆H₃₀N₄O₄ | 498 | | ES− 461.2 [M − H] | ¹H NMR (400 MHz, Methanol-d₄) δ 8.00 (s, 1H), 7.94-7.87 (m, 1H), 7.56 (d, J = 1.9 Hz, 1H), 7.14 (d, J = 7.9 Hz, 1H), 6.54 (d, J = 6.0 Hz, 2H), 6.39 (d, J = 8.5 Hz, 1H), 5.11-5.05 (m, 1H), 4.65- 4.35 (m, 1H), 4.02- 3.85 (m, 1H), 3.67 (s, 3H), 3.49-3.48 (m, 1H), 3.28- 2.75 (m, 5H), 2.39 (s, 3H), 2.18 (s, 3H), 1.64 (d, J = 6.6 Hz, 3H). |
| 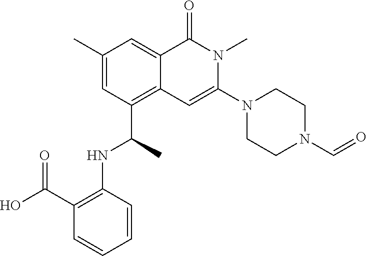 [SMILES: Cc1cc([C@@H](C)Nc2ccccc2C(=O)O)c2cc(N3CCN(C=O)CC3)n(C)c(=O)c2c1] C₂₅H₂₈N₄O₄ | 499 | | ES− 447.3 [M − H] | ¹H NMR (400 MHz, DMSO-d₆) δ 12.70 (s, 1H), 8.51 (s, 1H), 8.11 (s, 1H), 7.88 (s, 1H), 7.84-7.75 (m, 1H), 7.43 (d, J = 1.9 Hz, 1H), 7.21-7.11 (m, 1H), 6.56-6.46 (m, 1H), 6.42 (s, 1H), 6.36 (d, J = 8.5 Hz, 1H), 5.14-5.10 (m, 1H), 4.22-4.10 (m, 1H), 3.88-3.60 (m, 1H), 3.55 (s, 3H), 3.40-2.74 (m, 6H), 2.33 (s, 3H), 1.53 (d, J = 6.6 Hz, 3H). |
| 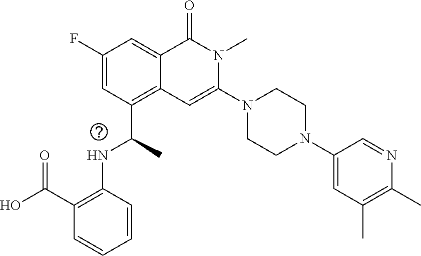 C₂₉H₂₉F₂N₅O₃ | 500 | | ES+ 534.5 [M + H] | ¹H NMR (400 MHz, DMSO-d₆) δ 12.81 (s, 1H), 8.44 (s, 1H), 8.16-8.11 (m, 1H), 7.83-7.81 (m, 1H), 7.74-7.71 (m, 1H), 7.40-7.30 (m, 2H), 7.21- 7.18 (m, 1H), 6.57- 6.53 (m, 2H), 6.36-6.28 (m, 1H), 5.28-5.25 (m, 1H), 3.85-3.57 (m, 5H), 3.32 (s, 3H), 3.25-2.89 (m, 3H), 2.35 (s, 3H), 1.57 (d, J = 6.6 Hz, 3H). |
| 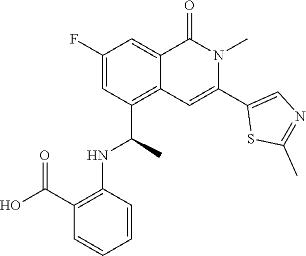 [SMILES: Cc1ncc(-c2cc3c([C@@H](C)Nc4ccccc4C(=O)O)cc(F)cc3c(=O)n2C)s1] C₂₃H₂₀FN₃O₃S | 501 | | ES+ 438.2 [M + H] | ¹H NMR (400 MHz, Methanol-d₄) δ 7.97- 7.85 (m, 3H), 7.53-7.49 (m, 1H), 7.21-7.12 (m, 2H), 6.62-6.53 (m, 1H), 6.29-6.22 (m, 1H), 5.20- 5.10 (m, 1H), 3.58 (s, 3H), 2.85 (s, 3H), 1.66- 1.59 (m, 3H). |
| 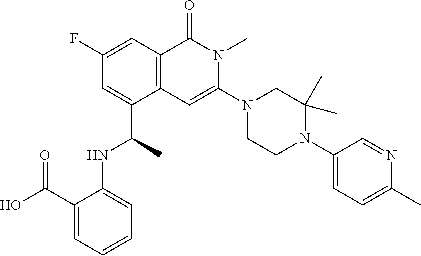 [SMILES: Cc1ccc(N2CCN(c3cc4c([C@@H](C)Nc5ccccc5C(=O)O)cc(F)cc4c(=O)n3C)CC2(C)C)cn1] C₃₁H₃₄FN₅O₃ | 502 | | ES+ 544.2 [M + H] | ¹H NMR (400 MHz, DMSO-d₆) δ 13.02-12.41 (m, 1H), 8.42 (s, 1H), 8.26 (d, J = 2.6 Hz, 1H), 7.84- 7.80 (m, 1H), 7.77-7.70 (m, 1H), 7.55-7.48 (m, 1H), 7.43.7.36 (m, 1H), 7.25-7.18 (m, 2H), 6.59- 7.52 (m, 1H), 6.48 (s, 1H), 6.38 (d, J = 8.5 Hz, 1H), 5.27 (s, 1H), 3.62 (s, 3H), 3.16 (s, 6H), 2.44 (s, 3H), 1.58 (d, J = 6.5 Hz, 3H), 1.11 (s, 6H). |
| 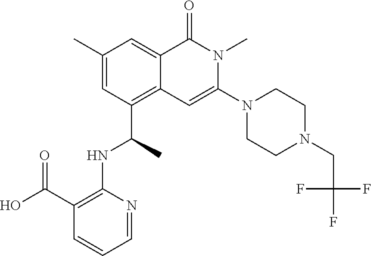 [SMILES: Cc1cc([C@@H](C)Nc2ncccc2C(=O)O)c2cc(N3CCN(CC(F)(F)F)CC3)n(C)c(=O)c2c1] C₂₅H₂₈F₃N₅O₃ | 503 | | ES− 502.2 [M − H] | ¹H NMR (400 MHz, DMSO-d₆) δ 13.03 (s, 1H), 8.44 (d, J = 7.7 Hz, 1H), 8.27-8.21 (m, 1H), 8.12- 8.05 (m, 1H), 7.89 (s, 1H), 7.49 (d, J = 1.9 Hz, 1H), 6.65-6.57 (m, 1H), 6.25 (s, 1H), 5.88-5.78 (m, 1H), 3.40 (s, 3H), 3.33- 3.21 (m, 2H), 2.78 (s, 8H), 2.38 (s, 3H), 1.57 (d, J = 6.7 Hz, 3H). |
| 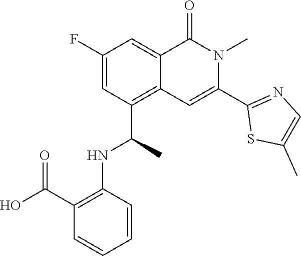 [SMILES: Cc1cnc(-c2cc3c([C@@H](C)Nc4ccccc4C(=O)O)cc(F)cc3c(=O)n2C)s1] C₂₃H₂₀FN₃O₃S | 504 | | ES+ 438.0 [M + H] | ¹H NMR (400 MHz, DMSO-d₆) δ 10.27 (d, J = 6.8 Hz, 1H), 7.84-7.76 (m, 3H), 7.54-7.51 (m, 1H), 7.26 (s, 1H), 6.86- 6.82 (m, 1H), 6.38-6.34 (m, 1H), 6.12 (d, J = 8.1 Hz, 1H), 5.19-5.14 (m, 1H), 3.59 (s, 3H), 2.56 (d, J = 1.2 Hz, 3H), 1.48 (d, J = 6.6 Hz, 3H). |
| 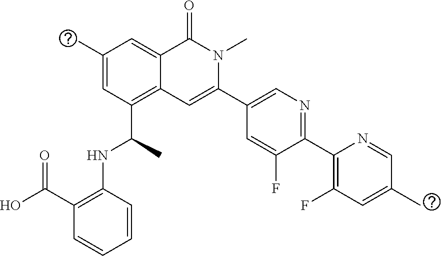 C₂₉H₂₀ClF₃N₄O₃ | 505 | | ES+ 565.2 [M + H] | ¹H NMR (400 MHz, DMSO-d₆) δ 12.81 (s, 1H), 9.51-8.41 (m, 1H), 9.12- 8.37 (m, 1H), 8.44 (s, 1H), 8.42-8.32 (m, 2H), 7.91-7.80 (m, 2H), 7.96- 7.01 (m, 1H), 7.23- 7.14 (m, 2H), 6.61-6.52 (m, 1H), 6.33 (d, J = 8.4 Hz, 1H), 5.37-5.27 (m, 1H), 3.32 (s, 3H), 1.56 (d, J = 6.5 Hz, 3H). |
| 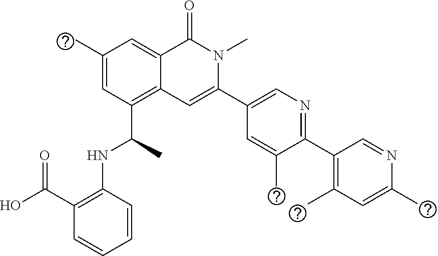 C₂₉H₂₀F₄N₄O₃ | 506 | | ES+ 549.2 [M + H] | ¹H NMR (400 MHz, DMSO-d₆) δ 12.91 (s, 1H), 8.97 (d, J = 1.6 Hz, 1H), 8.83 (s, 2H), 8.62 (s, 1H), 8.45-8,42 (m, 1H), 7.90- 7.75 (m, 2H), 7.46- 7.36 (m, 1H), 7.24 (s, 1H), 7.19-7.12 (m, 1H), 6.53- 6.48 ( m, 1H), 6.30 (d, J = 8.4 Hz, 1H), 5.38-5.28 (m, 1H), 3.46 (s, 3H), 1.54 (d, J = 6.5 Hz, 3H). |
| 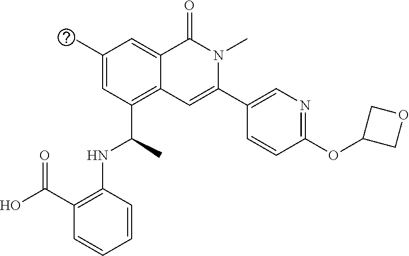 C₂₇H₂₄FN₃O₅ | 507 | | ES− 490.4 [M + H] | ¹H NMR (400 MHz, Methanol-d₄) δ 8.30 (d, J = 2.4 Hz, 1H), 7.97-7.86 (m, 3H), 7.53-7.49 (m, 1H), 7.18-7.10 (m, 1H), 7.04 (d, J = 8.5 Hz, 1H), 6.98 (s, 1H), 6.59-6.55 (m, 1H), 6.29 (d, J = 8.4 Hz, 1H), 5.74-5.69 (m, 1H), 5.24-5.19 (m, 1H), 5.08-5.03 (m, 2H), 4.79- 4.73 (m, 2H), 3.49 (s, 3H), 1.69-1.60 (m, 3H). |
| 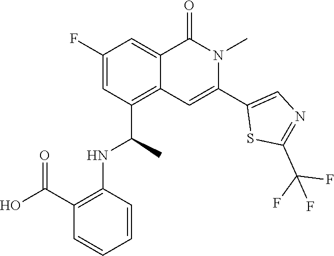 [SMILES: C[C@@H](Nc1ccccc1C(=O)O)c1cc(F)cc2c(=O)n(C)c(-c3cnc(C(F)(F)F)s3)cc12] C₂₃H₁₇F₄N₃O₃S | 508 | | ES+ 492.3 [M + H] | ¹H NMR (400 MHz, DMSO-d₆) δ 8.66 (s, 1H), 8.49 (s, 1H), 7.90-7.79 (m, 2H), 7.48-7.46 (m, 1H), 7.35 (s, 1H), 7.14- 7.12 (m, 1H), 6.54-6.53 (m, 1H), 6.29 (d, J = 8.5 Hz, 1H), 5.32-5.25(m, 1H), 3.49 (s, 3H), 1.53 (d, J = 6.6 Hz, 3H). |
| 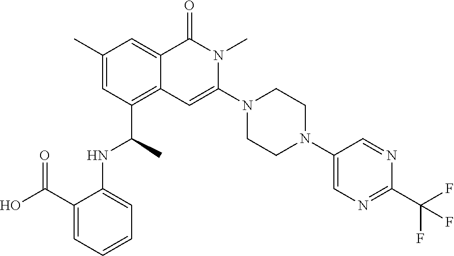 [SMILES: Cc1cc([C@@H](C)Nc2ccccc2C(=O)O)c2cc(N3CCN(c4cnc(C(F)(F)F)nc4)CC3)n(C)c(=O)c2c1] C₂₉H₂₉F₃N₆O₃ | 509 | | ES− 565.4 [M − H] | ¹H NMR (400 MHz, DMSO-d₆) δ 12.75 (s, 1H), 8.73 (s, 2H), 8.39 (d, J = 5.8 Hz, 1H), 7.90 (s, 1H), 7.79-7.78 (m, 1H), 7.43 (d, J = 1.9 Hz, 1H), 7.23- 7.14 (m, 1H), 6.51-6.50 (m, 1H), 6.46 (s, 1H), 6.38 (d, J = 8.5 Hz, 1H), 5.18- 5.17 (m, 1H), 4.25-3.75 (m, 2H), 3.56 (s, 3H), 3.32- 2.89 (m, 6H), 2.33 (s, 3H), 1.54 (d, J = 6.6 Hz, 3H). |
| 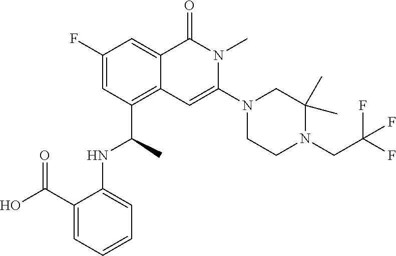 [SMILES: C[C@@H](Nc1ccccc1C(=O)O)c1cc(F)cc2c(=O)n(C)c(N3CCN(CC(F)(F)F)C(C)(C)C3)cc12] C₂₇H₃₀F₄N₄O₃ | 510 | | ES+ 535.2 [M + H] | ¹H NMR (400 MHz, DMSO-d₆) δ 12.79 (s, 1H), 8.39 (s, 1H), 7.85-7.79 (m, 1H), 7.74-7.68 (m, 1H), 7.39-7.33 (m, 1H), 7.25-7.16 (m, 1H), 6.58- 6.51 (m, 1H), 6.45 (s, 1H), 6.36 (d, J = 8.5 Hz, 1H), 5.31-5.22 (m, 1H), 3.56 (s, 3H), 3.32-2.59 (m, 8H), 1.55 (d, J = 6.5 Hz, 3H), 1.13 (d, J = 8.0 Hz, 6H). |
| 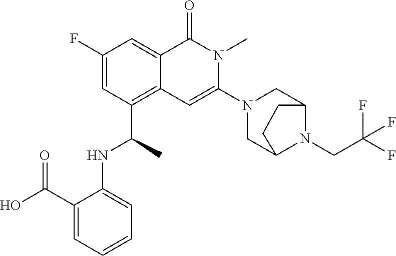 [SMILES: C[C@@H](Nc1ccccc1C(=O)O)c1cc(F)cc2c(=O)n(C)c(N3CC4CCC(C3)N4CC(F)(F)F)cc12] C₂₇H₂₈F₄N₄O₃ | 511 | | ES+ 533.5 [M + H] | ¹H NMR (400 MHz, DMSO-d₆) δ 12.71 (s, 1H), 8.48 (s, 1H), 7.82-7.81 (m, 1H), 7.70-7.68 (m, 1H), 7.34-7.32 (m, 1H), 7.18-7.15 (m, 1H), 6.58- 6.50 (m, 2H), 6.31 (d, J = 8.5 Hz, 1H), 5.30-5.25 (m, 1H), 3.57 (s, 3H), 3.41- 3.34 (m, 2H), 3.19- 3.07 (m, 3H), 3.04-2.90 (m, 3H), 1.93 (s, 4H), 1.53 (d, J = 6.5 Hz, 3H). |
| 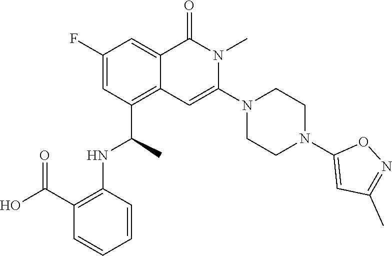 [SMILES: Cc1cc(N2CCN(c3cc4c([C@@H](C)Nc5ccccc5C(=O)O)cc(F)cc4c(=O)n3C)CC2)on1] C₂₇H₂₈FN₅O₄ | 512 | | ES− 504.2 [M − H] | ¹H NMR (400 MHz, DMSO-d₆) δ 8.72 (s, 1H), 7.84-7.77 (m, 1H), 7.74- 7.67 (m, 1H), 7.42-7.34 (m, 1H), 7.17-7.09 (m, 1H), 6.55-6.47 (m, 2H), 6.30 (d, J = 8.4 Hz, 1H), 5.27 (s, 1H), 5.26-5.19 (m, 1H), 3.55 (s, 3H), 3.34- 2.50 (m, 8H), 2.09 (s, 3H), 1.54 (d, J = 6.5 Hz, 3H). |
| 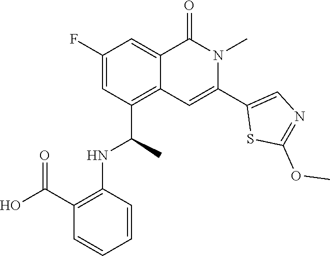 [SMILES: COc1ncc(-c2cc3c([C@@H](C)Nc4ccccc4C(=O)O)cc(F)cc3c(=O)n2C)s1] C₂₃H₂₀FN₃O_{4S} | 513 | | ES− 452.1 [M − H] | ¹H NMR (400 MHz, DMSO-d₆) δ 8.47 (s, 1H), 7.86-7.78 (m, 2H), 7.53 (s, 1H), 7.49-7.42 (m, 1H), 7.16 (d, J = 10.8 Hz, 2H), 6.59-6.51 (m, 1H), 6.31 (d, J = 8.5 Hz, 1H), 5.29 (d, J = 7.2 Hz, 1H), 4.11 (s, 3H), 3.50 (s, 3H), 1.54 (d, J = 6.5 Hz, 3H). |
| 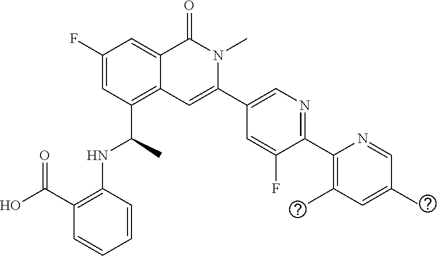 C₂₉H₂₀ClF₃N₄O₃ | 514 | | ES+ 565.2 [M + H] | ¹H NMR (400 MHz, DMSO-d₆) δ 12.81 (s, 1H), 8.90-8.85 (m, 1H), 8.81 (d, J = 2.5 Hz, 1H), 8.45- 8.31 (m, 3H), 7.90-7.85 (m, 1H), 7.84-7.81 (m, 1H), 7.50-7.40 (m, 1H), 7.25-7.12 (m, 2H), 6.60- 6.51 (m, 1H), 6.33 (d, J = 8.5 Hz, 1H), 5.46-5.33 (m, 1H), 3.45 (s, 3H), 1.56 (d, J = 6.5 Hz, 3H). |
| 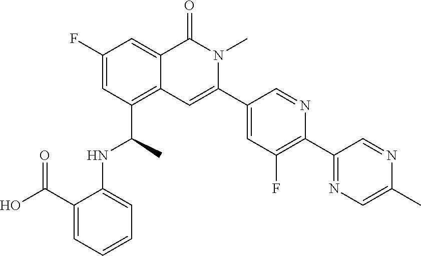 [SMILES: Cc1cnc(-c2ncc(-c3cc4c([C@@H](C)Nc5ccccc5C(=O)O)cc(F)cc4c(=O)n3C)cc2F)cn1] C₂₉H₂₃F₂N₅O₃ | 515 | | ES+ 528.2 [M + H] | ¹H NMR (DMSO-d₆, 400 MHz, ppm): δ 12.84 (s, 1H), 9.17 (d, J = 1.4 Hz, 1H), 8.91-8.86 (m, 1H), 8.74 (s, 1H), 8.45 (s, 1H), 8.35-8.27 (m, 1H), 7.91- 7.79 (m, 2H), 7.50- 7.41 (m, 1H), 7.17 (s, 2H), 6.60-6.50 (m, 1H), 6.32 (d, J = 8.5 Hz, 1H), 5.36- 5.25 (m, 1H), 3.46 (s, 3H), 2.62 (s, 3H), 1.55 (d, J = 6.6 Hz, 3H). |
| 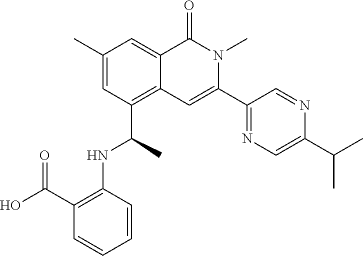 [SMILES: Cc1cc([C@@H](C)Nc2ccccc2C(=O)O)c2cc(-c3cnc(C(C)C)cn3)n(C)c(=O)c2c1] C₂₇H₂₈N₄O₃ | 516 | | ES+ 457.1 [M + H] | ¹H NMR (400 MHz, DMSO-d₆) δ 12.70 (s, 1H), 8.96 (d, J = 1.4 Hz, 1H), 8.77 (d, J = 1.4 Hz, 1H), 8.55 (s, 1H), 8.02 (s, 1H), 7.81-7.79 (m, 1H), 7.52 (d, J = 1.8 Hz, 1H), 7.19- 7.15 (m, 2H), 6.51-6.48 (m, 1H), 6.34 (d, J = 8.5 Hz, 1H), 5.29-5.25 (m, 1H), 3.44 (s, 3H), 3.24- 3.20 (m, 1H), 2.39 (s, 3H), 1.52 (d, J = 6.5 Hz, 3H), 1.35 (s, 3H), 1.34 (s, 3H). |
| 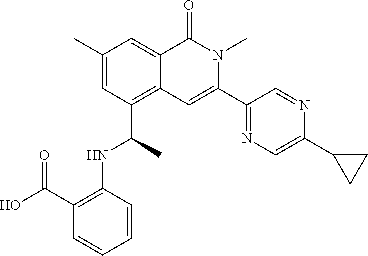 [SMILES: Cc1cc([C@@H](C)Nc2ccccc2C(=O)O)c2cc(-c3cnc(C4CC4)cn3)n(C)c(=O)c2c1] C₂₇H₂₆N₄O₃ | 517 | | ES− 453.4 [M − H] | ¹H NMR (400 MHz, Methanol-d₄) δ 8.75 (d, J = 1.4 Hz, 1H), 8.69 (d, J = 1.5 Hz, 1H), 8.12 (s, 1H), 7.94-7.87 (m, 1H), 7.64 (d, J = 1.8 Hz, 1H), 7.18- 7.06 (m, 2H), 6.56-6.50 (m, 1H), 6.34 (d, J = 8.4 Hz, 1H), 5.24-5.17 (m, 1H), 3.54 (s, 3H), 2.44 (s, 3H), 2.36-2.24 (m, 1H), 1.63 (d, J = 6.7 Hz, 3H), 1.24-1.10 (m, 4H). |
| 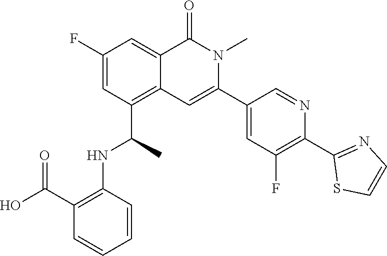 [SMILES: C[C@@H](Nc1ccccc1C(=O)O)c1cc(F)cc2c(=O)n(C)c(-c3cnc(-c4nccs4)c(F)c3)cc12] C₂₇H₂₀F₂N₄O₃S | 518 | | ES+ 519.3 [M + H] | ¹H NMR (Methanol-d₄, 400 MHz, ppm): δ 8.78- 8.67 (m, 1H), 8.15-8.06 (m, 2H), 7.98-7.91 (m, 2H), 7.87 (d, J = 3.3 Hz, 1H), 7.50-7.59 (m, 1H), 7.22-7.10 (m, 2H), 6.62- 6.55 (m, 1H), 6.33 (d, J = 8.4 Hz, 1H), 5.32-5.21 (m, 1H), 3.56 (s, 3H), 1.66 (d, J = 6.7 Hz, 3H). |
| 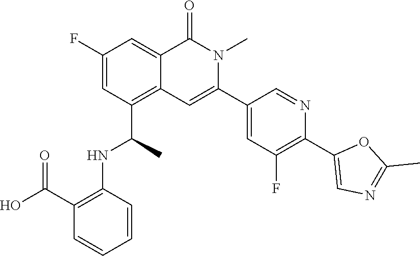 [SMILES: Cc1ncc(-c2ncc(-c3cc4c([C@@H](C)Nc5ccccc5C(=O)O)cc(F)cc4c(=O)n3C)cc2F)o1] C₂₈H₂₂F₂N₄O₃S | 519 | | ES+ 533.1 [M + H] | ¹H NMR (400 MHz, DMSO-d₆) δ 12.82 (s, 1H), 8.74 (d, J = 1.8 Hz, 1H), 8.44 (s, 1H), 8.33-8.25 (m, 2H), 7.89-7.79 (m, 2H), 7.48-7.42 (m, 1H), 7.22-7.12 (m, 2H), 6.58- 6.52 (m, 1H), 6.31 (d, J = 8.5 Hz, 1H), 5.38-5.30 (m, 1H), 3.44 (s, 3H), 2.75 (s, 3H), 1.54 (d, J = 6.5 Hz, 3H). |
| 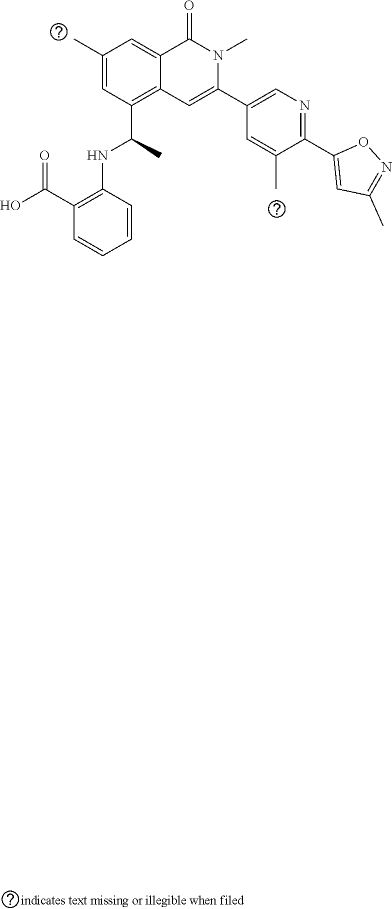 C₂₈H₂₂F₂N₄O₄ | 520 | | ES+ 517.3 [M + H] | ¹H NMR (400 MHz, DMSO-d₆) δ 8.88 (t, J = 1.7 Hz, 1H), 8.43 (s, 1H), 8.38-3.35 (m, 1H), 7.90- 7.79 (m, 2H), 7.47-7.44 (m, 1H), 7.17 (s, 2H), 7.08 (d, J = 2.7 Hz, 1H), 6.57- 6.53 (m, 1H), 6.31 (d, J = 8.5 Hz, 1H), 5.34 (s, 1H), 3.44 (s, 3H), 2.38 (s, 3H), 1.54 (d, J = 6.5 Hz, 3H). |
| 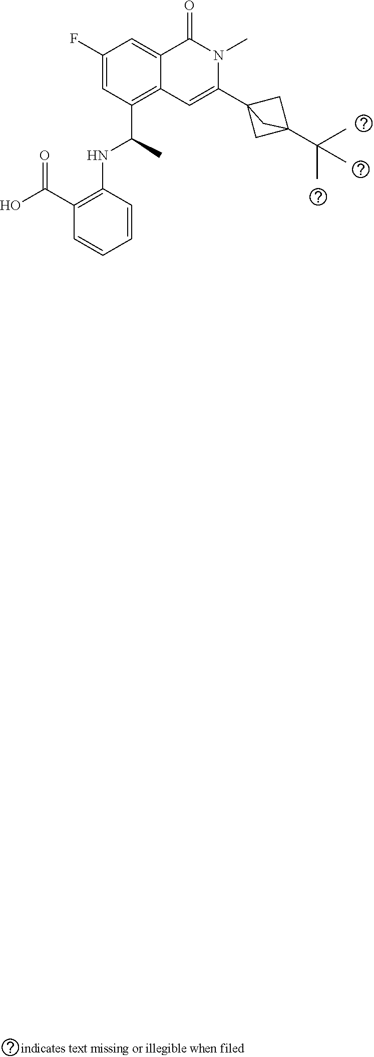 [SMILES: C[C@@H](Nc1ccccc1C(=O)O)c1cc(F)cc2c(=O)n(C)c(C34CC(C(C)(C)C)(C3)C4)cc12] C₂₅H₂₂F₄N₂O₃ | 521 | | ES− 473.2 [M − H] | ¹H NMR (400 MHz, DMSO-d₆) δ 12.83 (s, 1H), 8.39 (s, 1H), 7.85-7.73 (m, 2H), 7.51-7.42 (m, 1H), 7.25-7.17 (m, 1H), 6.70 (s, 1H), 6.6-6.52 (m, 1H), 6.37 (d, J = 8.5 Hz, 1H), 5.31-5.23 (m, 1H), 3.62 (s, 3H), 2.59-2.47 (m, 6H), 1.57 (d, J = 6.5 Hz, 3H). |
| 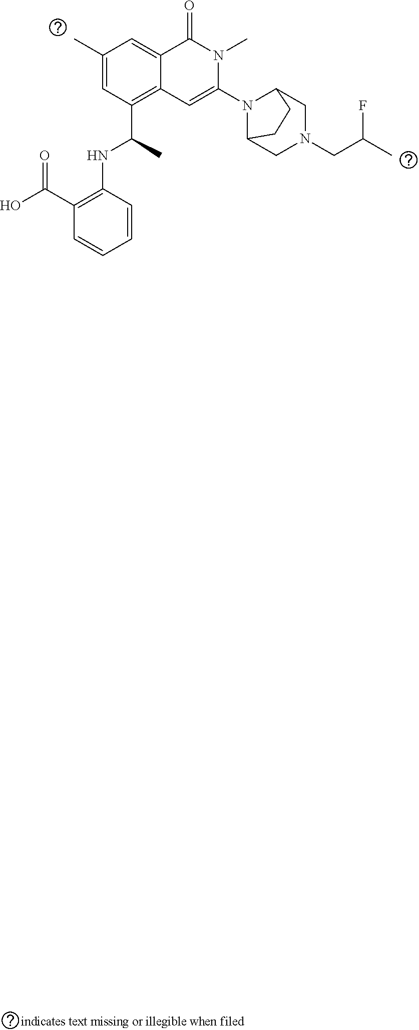 C₂₇H₂₉F₃N₄O₃ | 522 | | ES+ 515.1 [M + H] | ¹H NMR (400 MHz, DMSO-d₆) δ 12.74 (S, 1H), 8.35 (s, 1H), 7.85-7.78 (m, 1H), 7.72-7.64 (m, 1H), 7.41-7.33 (m, 1H), 7.21 (t, J = 8.2 Hz, 1H), 6.55 (t, J = 7.5 Hz, 1H), 6.40 (d, J = 8.5 Hz, 1H), 6.17 (s, 1H), 5.19 (s, 1H), 3.90 (d, J = 6.0 Hz, 1H), 3.83 (s, 1H), 3.58 (s, 3H), 2.89-2.76 (m, 3H), 2.77 (s, 1H), 2.67 (t, J = 11.7 Hz, 2H), 1.95 (s, 1H), 1.87 (d, J = 16.7 Hz, 4H), 1.55 (d, J = 6.6 Hz, 3H). |
| 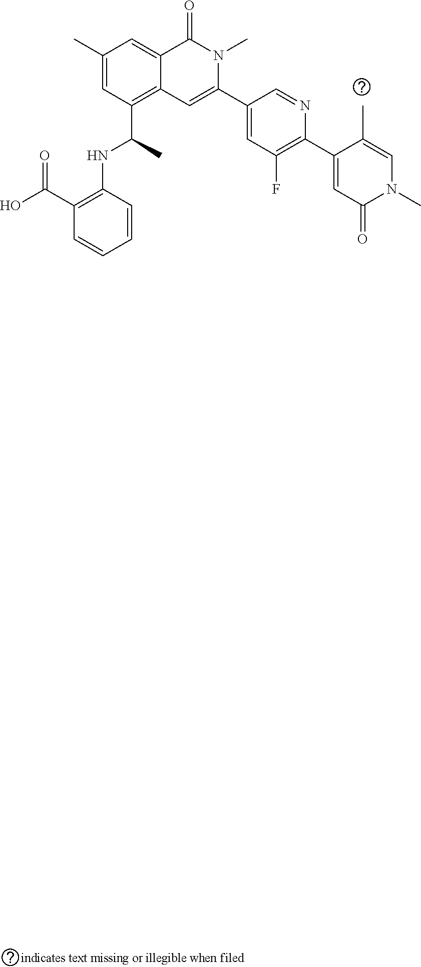 C₃₁H₂₆F₂N₄O₄ | 523 | | ES− 557.6 [M + H] | ¹H NMR (400 MHz, DMSO-d₆) δ 12.76 (s, 1H), 8.90-8.80 (m, 1H), 8.40 (d, J = 5.8 Hz, 1H), 8.31- 8.25 (m, 1H), 8.20 (d, J = 6.5 Hz, 1H), 8.05 (s, 1H), 7.82-7.79(m, 1H), 7.54 (d, J = 1.9 Hz, 1H), 7.26 (s, 1H), 7.20-7.10 (m, 1H), 6.80 (d, J = 7.5 Hz, 1H), 6.58-6.49 (m, 1H), 6.32 (d, J = 8.5 Hz, 1H), 5.27- 5.20 (m, 1H), 3.48 (s, 3H), 3.32 (s, 3H), 2.40 (s, 3H), 1.51 (d, J = 6.5 Hz, 3H). |
| 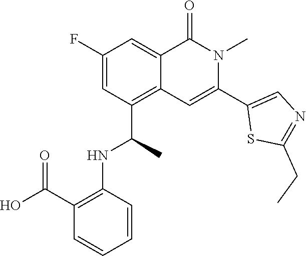 [SMILES: CCc1ncc(-c2cc3c([C@@H](C)Nc4ccccc4C(=O)O)cc(F)cc3c(=O)n2C)s1] C₂₄H₂₂FN₃O₃S | 524 | | ES− 450.2 [M − H] | ¹H NMR (400 MHz, DMSO-d₆) δ 12.84 (s, 1H), 8.46 (s, 1H), 7.98 (s, 1H), 8.31-7.43 (m, 2H), 7.55- 7.06 (m, 1H), 7.16 (d, J = 6.1 Hz, 2H), 6.59-6.50 (m, 1H), 6.30 (d, J = 8.5 Hz, 1H), 5.34-5.26 (m, 1H), 3.47 (s, 3H), 3.48- 2.79 (m, 2H), 1.53 (d, J = 6.6 Hz, 3H), 1.87-1.03 (m, 3H). |
| 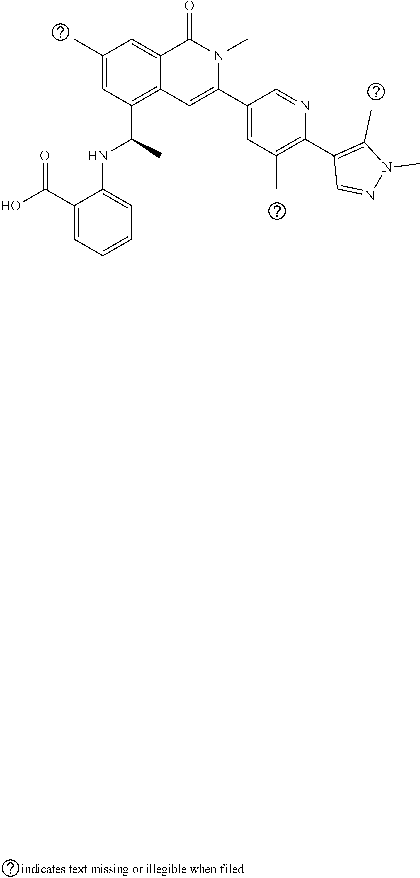 [SMILES: Cc1cc([C@@H](C)Nc2ccccc2C(=O)O)c2cc(-c3cnc(-c4cnn(C)c4C)c(C)c3)n(C)c(=O)c2c1] C₂₈H₂₂F₃N₅O₃ | 525 | | ES− 532.2 [M − H] | ¹H NMR (400 MHz, Methanol-d₄) δ 8.63- 8.58 (m, 1H), 8.01-7.88 (m, 4H), 7.52-7.45 (m, 1H), 7.15-7.05 (m, 1H), 7.09 (s, 1H), 6.61-6.55 (m, 1H), 6.32 (d, J = 8.4 Hz, 1H), 5.24-5.19 (m, 1H), 3.86 (d, J = 1.4 Hz, 3H), 3.53 (s, 3H), 1.64 (d, J = 6.6 Hz, 3H). |
| 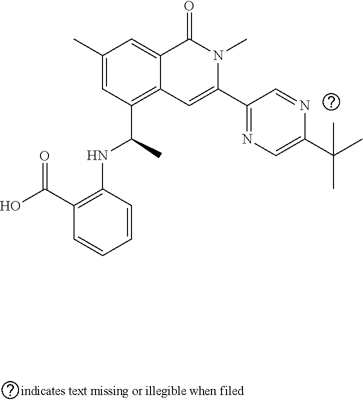 C₂₇H₂₇FN₄O₃ | 526 | | ES+ 475.1 [M + H] | ¹H NMR (400 MHz, Methanol-d₄) δ 8.97- 8.92 (m, 2H), 8.13 (d, J = 1.7 Hz, 1H), 7.94-7.90 (m, 1H), 7.64 (d, J = 1.9 Hz, 1H), 7.22 (s, 1H), 7.14- 7.09 (m, 1H), 6.57- 6.49 (m, 1H), 6.35 (d, J = 8.5 Hz, 1H), 5.27-5.22 (m, 1H), 3.57 (s, 3H), 2.45 (s, 3H), 1.83 (s, 3H), 1.78 (s, 3H), 1.63 (d, J = 6.7 Hz, 3H).5 |
| 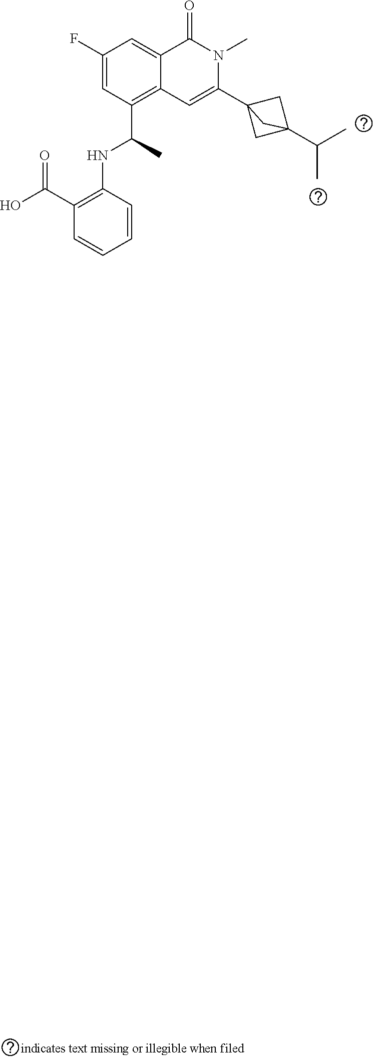 C₂₅H₂₃F₃N₂O₃ | 527 | | ES+ 457.2 [M + H] | ¹H NMR (400 MHz, DMSO-d₆) δ 12.81 (s, 1H), 8.47 (s, 1H), 7.82-7.80 (m, 1H), 7.76-7.70 (m, 1H), 7.46-7.41 (m, 1H), 7.20-7.15 (m, 1H), 6.68 (s, 1H), 6.60-6.51 (m, 1H), 6.40-6.30 (m, 1H), 6.18 (s, 1H), 5.25-5.20 (m, 1H), 3.62 (s, 3H), 2.33- 2.25 (m, 6H), 1.57 (d, J = 6.6 Hz, 3H). |
| 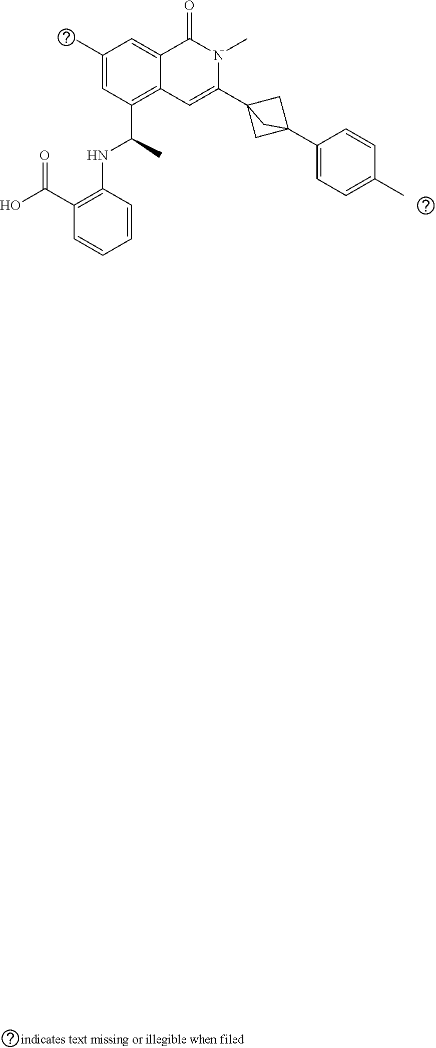 C₃₀H₂₆F₂N₂O₃ | 528 | | ES+ 501.2 [M + H] | ¹H NMR (Methanol-d₄, 400 MHz, ppm): δ 7.99- 7.91 (m, 1H), 7.89-7.81 (m, 1H), 7.54-7.51 (m, 1H), 7.39-7.31 (m, 2H), 7.19-7.14 (m, 1H), 7.12- 7.03 (m, 2H), 6.86 (s, 1H), 6.61-6.55 (m, 1H), 6.37 (d, J = 8.5 Hz, 1H), 5.30-5.12 (m, 1H), 3.81 (s, 3H), 2.56-2.54 (m, 6H), 1.69 (d, J = 6.7 Hz, 3H) |
| 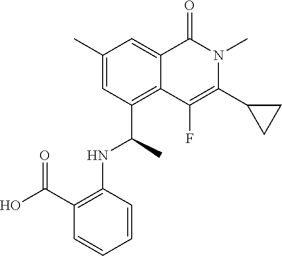 [SMILES: Cc1cc([C@@H](C)Nc2ccccc2C(=O)O)c2c(F)c(C3CC3)n(C)c(=O)c2c1] C₂₃H₂₃FN₂O₃ | 529 | | ES− 393.2 [M − H] | ¹H NMR (400 MHz, DMSO-d₆) δ 12.78 (s, 1H), 8.46-8.41 (m, 1H), 8.02 (d, J = 2.4 Hz, 1H), 7.82- 7.81 (m, 1H), 7.60 (s, 1H), 7.18-7.15 (m, 1H), 6.54- 6.51 (m, 1H), 6.28 (d, J = 8.5 Hz, 1H), 5.38-5.30 (m, 1H), 3.67 (s, 3H), 2.34 (s, 3H), 1.92-1.90 (m, 1H), 1.54 (d, J = 6.4 Hz, 3H), 1.16 (d, J = 7.9 Hz, 2H), 0.92 (d, J = 5.5 Hz, 2H). |
| 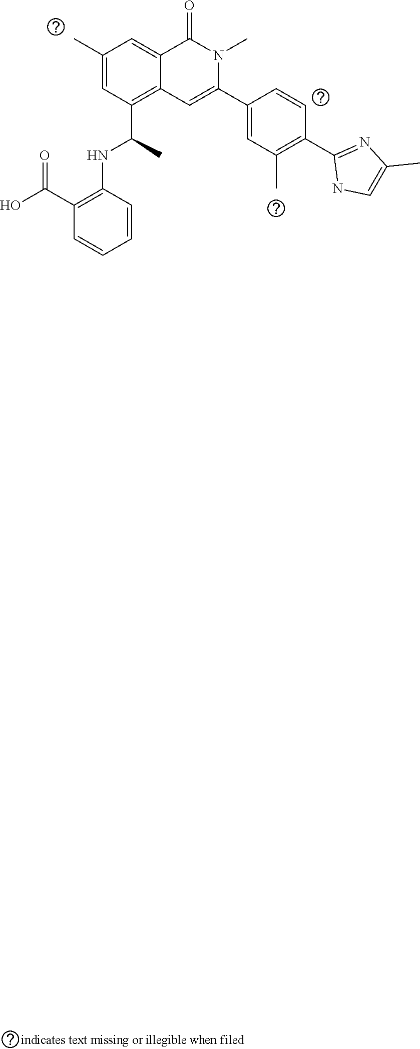 C₂₈H₂₂F₂N₄O₃S | 530 | | ES+ 533.2 [M + H] | ¹H NMR (400 MHz, DMSO-d₆) δ 12.86 (s, 1H), 8.82-8.77 (m, 1H), 8.43 (s, 1H), 8.34-8.27 (m, 1H), 7.90-7.79 (m, 2H), 7.57 (d, J = 1.2 Hz, 1H), 7.49- 7.42 (m, 1H), 7.23-7.14 (m, 1H), 7.18 (s, 1H), 6.60- 6.51 (m, 1H), 6.32 (d, J = 8.5 Hz, 1H), 5.35 (s, 1H), 3.45 (s, 3H), 2.54-2.46 (m, 3H), 1.55 (d, J = 6.6 Hz, 3H). |
| 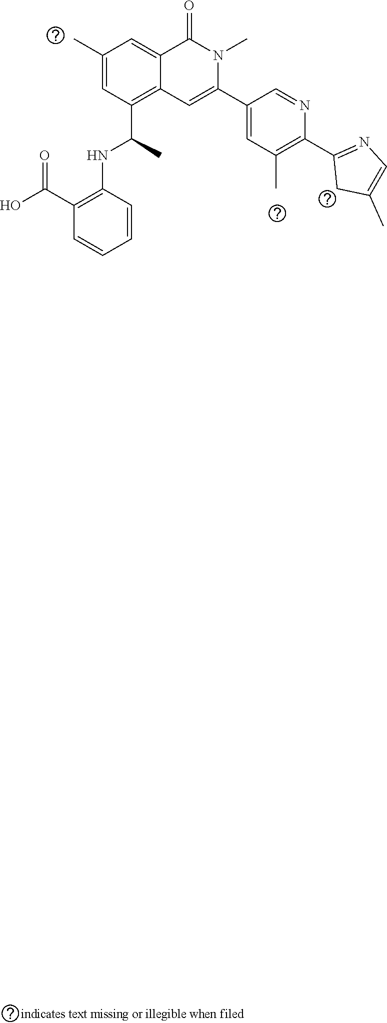 C₂₈H₂₂F₂N₄O₃S | 531 | | ES+ 533.2 [M + H] | ¹H NMR (400 MHz, DMSO-d₆) δ 8.78 (s, J = 1.5 Hz, 1H), 8.65-8.28 (m, 2H), 7.89-7.78 (m, 3H), 7.47-7.44 (m, 1H), 7.15 (d, J = 15.6 Hz, 2H), 6.57-6.50 (m, 1H), 6.30 (d, J = 8.5 Hz, 1H), 5.34 (d, J = 6.8 Hz, 1H), 3.33 (s, 3H), 2.56 (d, J = 1.2 Hz, 3H), 1.54 (d, J = 6.5 Hz, 3H). |
| 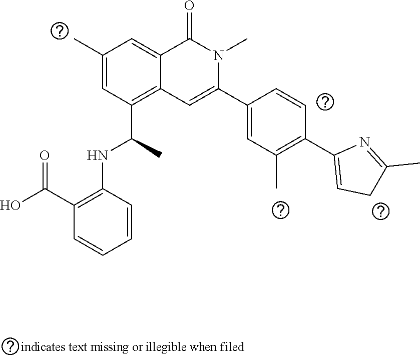 C₂₈H₂₂F₂N₄O₃S | 532 | | ES+ 533.1 [M + H] | ¹H NMR (400 MHz, DMSO-d₆) δ 8.77 (d, J = 1.7 Hz, 1H), 8.45 (s, 1H), 8.22 (d, J = 11.7 Hz, 2H), 7.89-7.78 (m, 2H), 7.49- 7.41 (m, 1H), 7.16 (d, J = 14.9 Hz, 2H), 6.58-6.50 (m, 1H), 6.32 (d, J = 8.5 Hz, 1H), 5.34 (s, 1H), 3.44 (s, 3H), 2.77 (s, 3H), 1.55 (d, J = 6.5 Hz, 3H). |
| 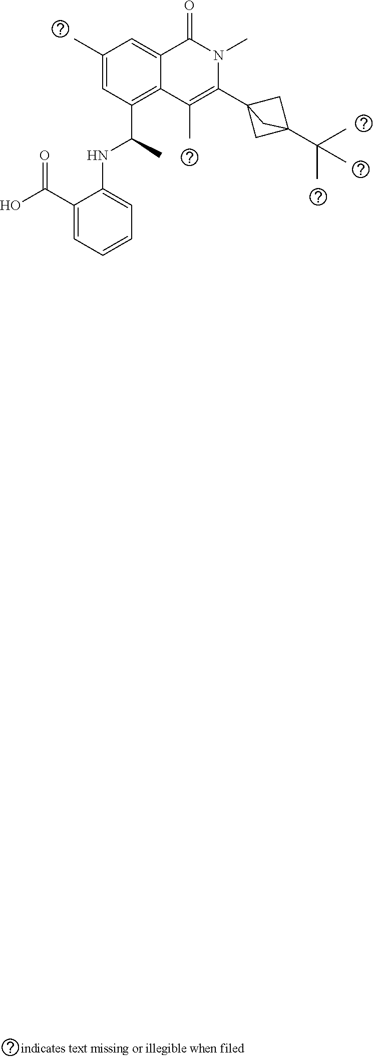 [SMILES: Cc1cc([C@@H](C)Nc2ccccc2C(=O)O)c2c(C)c(C34CC(C(C)(C)C)(C3)C4)n(C)c(=O)c2c1] C₂₅H₂₁F₅N₂O₃ | 533 | | ES− 491.2 [M − H] | ¹H NMR (400 MHz, DMSO-d₆) δ 8.62 (s, 1H), 7.91-7.81 (m, 2H), 7.58 (s, 1H), 7.20-7.12 (m, 1H), 6.60-6.52 (m, 1H), 6.22 (s, 1H), 5.29 (s, 1H), 3.63 (s, 3H), 2.71 (d, J = 1.4 Hz, 6H), 1.56 (d, J = 6.5 Hz, 3H). |
| 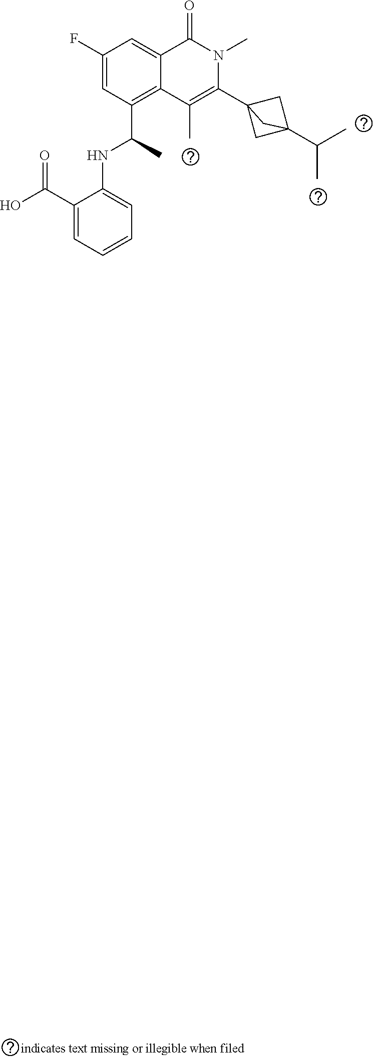 C₂₅H₂₂F₄N₂O₃ | 534 | | ES+ 475.1 [M + H] | ¹H NMR (400 MHz, DMSO-d₆) δ 12.89 (s, 1H), 8.46 (s, 1H), 7.86-7.75 (m, 2H), 7.57 (d, J = 10.0 Hz, 1H), 7.20-7.10 (m, 1H), 6.57-6.45 (m, 1H), 6.25 (d, J = 8.4 Hz, 1H), 6.17 (s, 1H), 5.32-5.22 (m, 1H), 3.63 (s, 3H), 2.54- 2.52 (m, 6H), 1.57 (d, J = 6.4 Hz, 3H). |
| 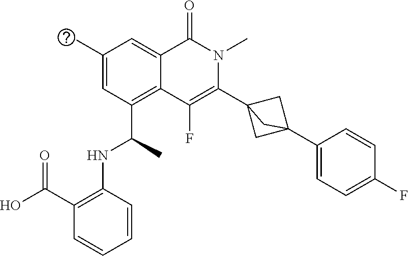 C₃₀H₂₅F₃N₂O₃ | 535 | | ES+ 519.2 [M + H] | ¹H NMR (Methanol-d₄, 400 MHz): δ 7.99-7.91 (m, 2H), 7.65 (d, J = 7.9 Hz, 1H), 7.40-7.32 (m, 2H), 7.17-7.13 (m, 1H), 7.08-7.02 (m, 2H), 6.61- 6.57 (m, 1H), 6.29 (d, J = 8.5 Hz, 1H), 5.46-5.35 (m, 1H), 3.82 (s, 3H), 2.80- 2.76 (m, 6H), 1.64 (d, J = 6.4 Hz, 3H). |
| 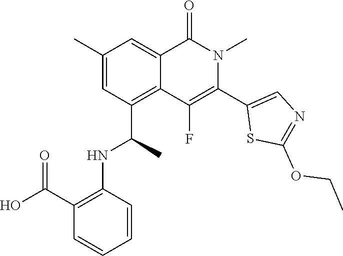 [SMILES: CCOc1ncc(-c2c(F)c3c([C@@H](C)Nc4ccccc4C(=O)O)cc(C)cc3c(=O)n2C)s1] C₂₅H₂₄FN₃O₄S | 536 | | ES− 480.3 [M − H] | ¹H NMR (400 MHz, DMSO-d₆) δ 12.82 (s, 1H), 8.45 (s, 1H), 8.10 (s, 1H), 7.83 (d, J = 7.9 Hz, 1H), 7.67 (s, 1H), 7.53 (s, 1H), 7.69-6.55 (m, 1H), 7.02- 6.05 (m, 1H), 6.29 (d, J = 8.5 Hz, 1H), 5.33-5.26 (m, 1H), 5.02-4.17 (m, 2H), 3.38 (s, 3H), 2.39 (s, 3H), 1.54 (d, J = 6.5 Hz, 3H), 1.93-1.12 (m, 3H). |
| 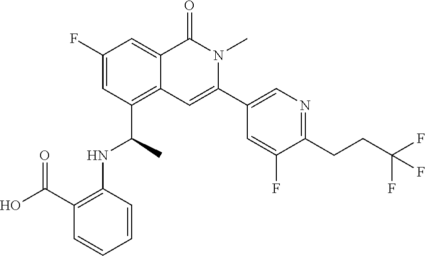 [SMILES: C[C@@H](Nc1ccccc1C(=O)O)c1cc(F)cc2c(=O)n(C)c(-c3cnc(CCC(F)(F)F)c(F)c3)cc12] C₂₆H₂₀F₅N₃O₄ | 537 | | ES− 532.2 [M − H] | ¹H NMR (400 MHz, Methanol-d₄) δ 8.22- 8.16 (m, 1H), 7.97-7.84 (m, 3H), 7.54-7.45 (m, 1H), 7.18-7.09 (m, 1H), 7.04 (s, 1H), 6.61-6.50 (m, 1H), 6.35-6.24 (m, 1H), 5.27-5.16 (m, 1H), 5.14-5.00 (m, 2H), 3.48 (d, J = 2.4 Hz, 3H), 1.70- 1.58 (m, 3H). |
| 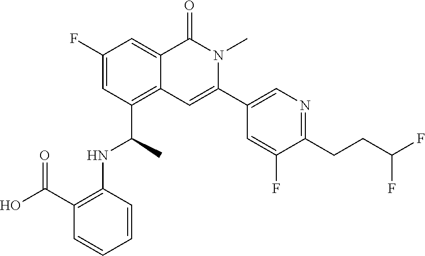 [SMILES: C[C@@H](Nc1ccccc1C(=O)O)c1cc(F)cc2c(=O)n(C)c(-c3cnc(CCC(F)F)c(F)c3)cc12] C₂₆H₂₁F₄N₃O₄ | 538 | | ES+ 516.2 [M + H] | ¹H NMR (400 MHz, Methanol-d₄) δ 8.18 (s, 1H), 7.97-7.82 (m, 3H), 7.57-7.49 (m, 1H), 7.14- 7.05 (m, 1H), 7.09- 7.01 (m, 1H), 6.60-6.50 (m, 1H), 6.34-6.24 (m, 2H), 5.19-5.18 (m, 1H), 4.80-4.67 (m, 2H), 3.33 (s, 3H), 1.62 (d, J = 6.0, 3H). |
| 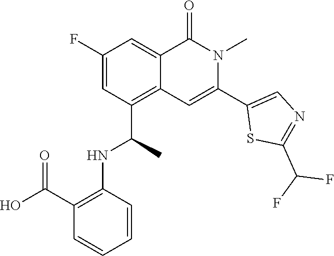 [SMILES: C[C@@H](Nc1ccccc1C(=O)O)c1cc(F)cc2c(=O)n(C)c(-c3cnc(C(F)F)s3)cc12] C₂₃H₁₈F₃N₃O₃S | 539 | | ES+ 474.3 [M + H] | ¹H NMR (400 MHz, DMSO-d₆) δ 13.05 (s, 1H), 8.57 (s, 1H), 8.36 (d, J = 1.7 Hz, 1H), 7.89-7.79 (m, 2H), 7.51-7.44 (m, 2H), 7.31 (d, J = 14.9 Hz, 1H), 7.15 (s, 1H), 6.58- 6.50 (m, 1H), 6.30 (d, J = 8.4 Hz, 1H), 5.33-5.25 (m, 1H), 3.48 (s, 3H), 1.53 (d, J = 6.5 Hz, 3H). |
| 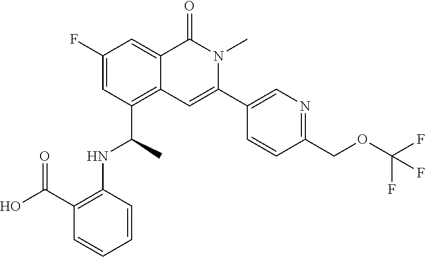 [SMILES: C[C@@H](Nc1ccccc1C(=O)O)c1cc(F)cc2c(=O)n(C)c(-c3ccc(COC(F)(F)F)nc3)cc12] C₂₆H₂₁F₄N₃O₄ | 540 | | ES+ 516.2 [M + H] | ¹H NMR (400 MHz, Methanol-d₄) δ 8.80 (d, J = 2.2 Hz, 1H), 8.21-8.06 (m, 1H), 7.96-7.88 (m, 2H), 7.73 (d, J = 8.1 Hz, 1H), 7.59-7.50 (m, 1H), 7.17-7.08 (m, 1H), 7.06 (s, 1H), 6.63-6.52 (m, 1H), 6.28 (d, J = 8.5 Hz, 1H), 5.28 (s, 2H), 5.27- 5.18 (m, 1H), 3.48 (s, 3H), 1.63 (d, J = 6.7 Hz, 3H). |
| 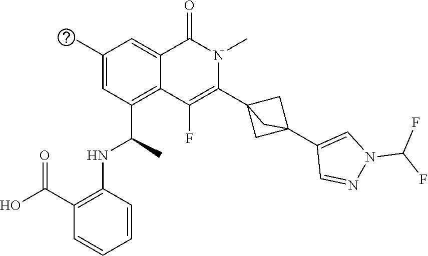 [SMILES: Cc1cc([C@@H](C)Nc2ccccc2C(=O)O)c2c(F)c(C34CC(c5cnn(C(F)F)c5)(C3)C4)n(C)c(=O)c2c1] C₂₈H₂₅F₃N₄O₃ | 541 | | ES− 521.0 [M − H] | ¹H NMR (400 MHz, DMSO-d₆) δ 12.81 (s, 1H), 8.43 (s, 1H), 8.15 (s, 1H), 7.86- 7.73 (m, 4H), 7.51- 7.44 (m, 1H), 7.26- 7.17 (m, 1H), 6.70 (s, 1H), 6.60-6.52 (m, 1H), 6.37 (d, J = 8.5 Hz, 1H), 5.28- 5.20 (m, 1H), 3.66 (s, 3H), 2.50 (s, 6H), 1.59 (d, J = 6.6 Hz, 3H). |
| 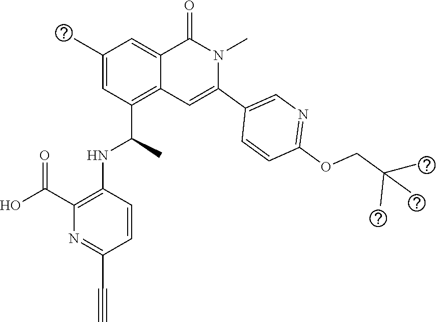 [SMILES: C#Cc1ccc(N[C@H](C)c2cc(C)cc3c(=O)n(C)c(-c4ccc(OCC(C)(C)C)nc4)cc23)c(C(=O)O)n1] C₂₇H₂₀F₄N₄O₄ | 542 | | ES+ 541.1 [M + H] | ¹H NMR (400 MHz, DMSO-d₆) δ 8.54 (s, 1H), 8.47 (d, J = 2.4 Hz, 1H), 8.13-8.08 (m, 1H), 7.88-7.84 (m, 1H), 7.52-7.48 (m, 1H), 7.38 (d, J = 8.8 Hz, 1H), 7.17 (d, J = 8.6 Hz, 1H), 6.96 (s, 1H), 6.76 (d, J = 8.9 Hz, 1H), 5.42- 5.35 (m, 1H), 5.14- 5.06 (m, 2H), 4.08 (s, 1H), 3.34 (s, 3H), 1.56 (d, J = 6.6 Hz, 3H). |
| 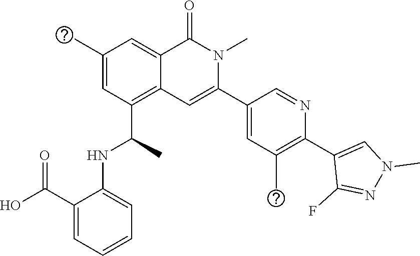 [SMILES: Cc1cc([C@@H](C)Nc2ccccc2C(=O)O)c2cc(-c3cnc(-c4cn(C)nc4F)c(C)c3)n(C)c(=O)c2c1] C₂₈H₂₂F₃N₅O₃ | 543 | | ES+ 534.3 [M + H] | ¹H NMR: (400 MHz, Methanol-d₄) δ 8.62 (s, 1H), 8.12 (s, 1H), 7.96-7.88 (m, 3H), 7.60-7.47 (m, 1H), 7.20-7.12 (m, 1H), 7.10 (s, 1H), 6.63- 6.50 (m, 1H), 6.32 (d, J = 8.5 Hz, 1H), 5.35-5.18 (m, 1H), 3.89 (s, 3H), 3.53 (s, 3H), 1.65 (d, J = 6.6 Hz, 3H). |
| 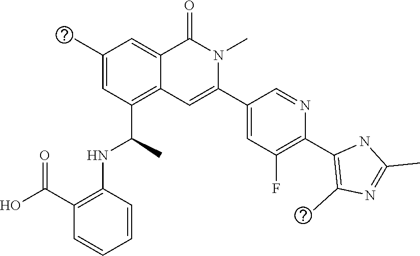 [SMILES: Cc1cc([C@@H](C)Nc2ccccc2C(=O)O)c2cc(-c3cnc(-c4[nH]c(C)nc4C)c(F)c3)n(C)c(=O)c2c1] C₂₈H₂₁F₃N₄O₃S | 544 | | ES− 549.3 [M − H] | ¹H NMR (400 MHz, DMSO-d₆) δ 12.83 (s, 1H), 8.78 (s, 1H), 8.40 (s, 1H), 8.30- 8.27 (m, 1H), 7.89- 7.79 (m, 2H), 7.46- 7.43 (m, 1H), 7.22- 7.12 (m, 2H), 6.58- 6.53 (m, 1H), 6.31 (d, J = 8.2 Hz, 1H), 5.37-5.32 (m, 1H), 3.44 (s, 3H), 2.71 (s, 3H), 1.54 (d, J = 6.5 Hz, 3H). |
| 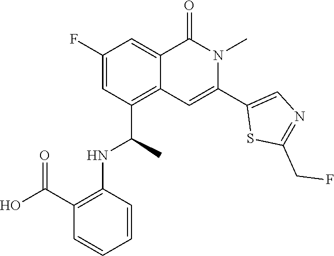 [SMILES: C[C@@H](Nc1ccccc1C(=O)O)c1cc(F)cc2c(=O)n(C)c(-c3cnc(CF)s3)cc12] C₂₃H₁₉F₂N₃O₃S | 545 | | ES+ 456.3 [M + H] | ¹H NMR (400 MHz, DMSO-d₆) δ 12.76 (s, 1H), 8.49 (s, 1H), 8.21 (s, 1H), 7.85- 7.81 (m, 2H), 7.47- 7.44 (m, 1H), 7.23- 7.16 (m, 2H), 6.56- 6.52 (m, 1H), 6.30 (d, J = 8.4 Hz, 1H), 5.85-5.84 (m, 1H), 5.74-5.73 (m, 1H), 5.32-5.31 (m, 1H), 3.47 (s, 3H), 1.53 (d, J = 6.5 Hz, 3H). |
| 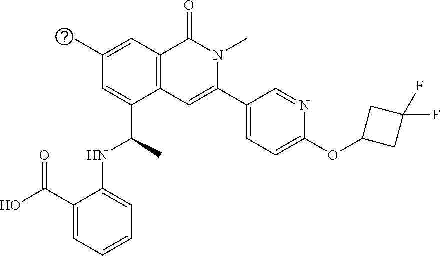 C₂₈H₂₄F₃N₃O₄ | 546 | | ES+ 524.1 [M + H] | ¹H NMR (400 MHz, Methanol-d₄) δ 8.35- 8.34 (m, 1H), 7.94- 7.90 (m, 3H), 7.54- 7.49 (m, 1H), 7.17- 7.13 (m, 1H), 7.01- 6.99 (m, 2H), 6.59- 6.55 (m, 1H), 6.31- 6.27 (m, 1H), 5.26- 5.18 (m, 2H), 3.48 (s, 3H), 3.25-3.11 (m, 2H), 2.86-2.69 (m, 2H), 1.64 (d, J = 6.6 Hz, 3H). |
| 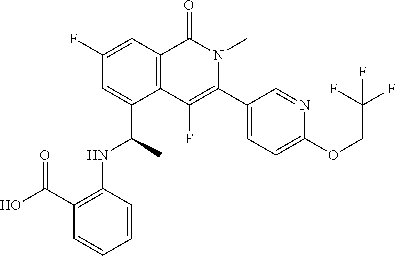 [SMILES: C[C@@H](Nc1ccccc1C(=O)O)c1cc(F)cc2c(=O)n(C)c(-c3ccc(OCC(F)(F)F)nc3)c(F)c12] C₂₆H₂₀F₅N₃O₄ | 547 | | ES− 532.0 [M − H] | ¹H NMR (400 MHz, DMSO-d₆) δ 8.49 (s, 1H), 8.47-8.41 (m, 1H), 8.11 (d, J = 8.3 Hz, 1H), 7.97 (d, J = 8.5 Hz, 1H), 7.84 (d, J = 7.9 Hz, 1H), 7.60 (d, J = 10.0 Hz, 1H), 7.26-7.15 (m, 2H), 6.61-6.53 (m, 1H), 6.30 (s, 1H), 5.34- 5.27 (m, 1H), 5.15- 5.03 (m, 2H), 3.26 (s, 3H), 1.55 (d, J = 6.4 Hz, 3H). |
| 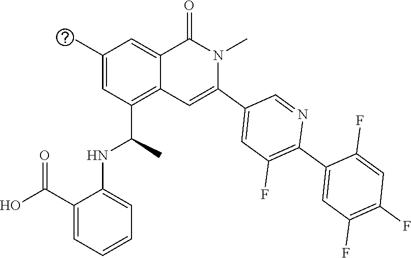 C₃₀H₂₀F₅N₃O₃ | 548 | | ES+ 566.2 [M + H] | ¹H NMR (400 MHz, DMSO-d₆) δ 12.79 (s, 1H), 8.89 (d, J = 1.7 Hz, 1H), 8.41 (d, J = 6.0 Hz, 1H), 8.35- 8.30 (m, 1H), 7.91- 7.76 (m, 4H), 7.49- 7.42 (m, 1H), 7.23- 7.15 (m, 2H), 6.59- 6.53 (m, 1H), 6.32 (d, J = 8.5 Hz, 1H), 5.41-5.33 (m, 1H), 3.45 (s, 3H), 1.55 (d, J = 6.6 Hz, 3H). |
| 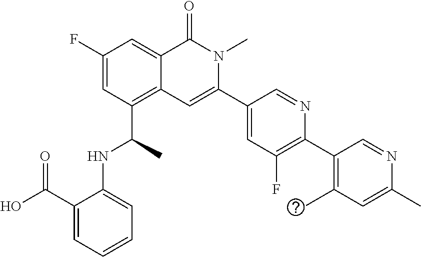 C₃₀H₂₃F₃N₄O₃ | 549 | | ES+ 545.4 [M + H] | ¹H NMR (400 MHz, DMSO-d₆) δ 12.81 (s, 1H), 8.90 (s, 1H), 8.82 (d, J = 11.0 Hz, 1H), 8.43 (d, J = 8.4 Hz, 1H), 8.35 (d, J = 10.8 Hz, 1H), 7.92- 7.89 (m, 1H), 7.85- 7.82 (m, 1H), 7.53- 7.44 (m, 2H), 7.33 (s, 1H), 7.26-7.14 (m, 1H), 6.58-6.55 (m, 1H), 6.29 (d, J = 8.5 Hz, 1H), 5.33- 5.30 (m, 1H), 3.42 (s, 3H), 2.59 (s, 3H), 1.54 (d, J = 6.5 Hz, 3H). |
| 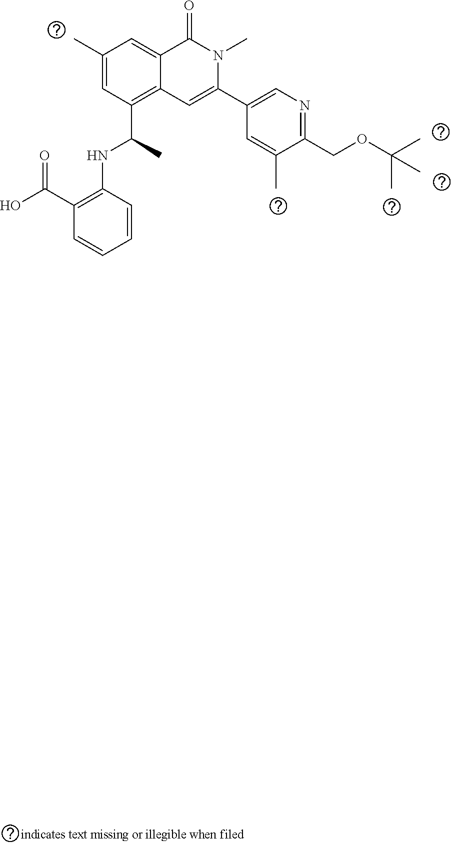 C₂₆H₂₀F₅N₃O₄ | 550 | | ES+ 534.5 [M + H] | ¹H NMR (400 MHz, Methanol-d₄) δ 8.69 (s, 1H), 8.07-8.01 (m, 1H), 7.93-7.91 (m, 2H), 7.54-7.51 (m, 1H), 7.16-7.08 (m, 2H), 6.58-6.55 (m, 1H), 6.32-6.28 (m, 1H), 5.34 (d, J = 2.0 Hz, 2H), 5.24- 5.16 (m, 1H), 3.50 (s, 3H), 1.64 (d, J = 6.7 Hz, 3H). |
| 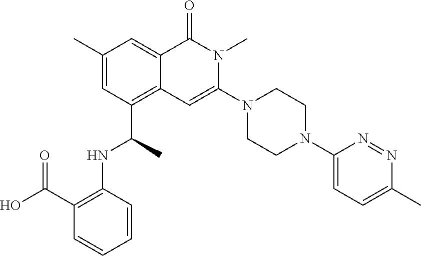 [SMILES: Cc1cc([C@@H](C)Nc2ccccc2C(=O)O)c2cc(N3CCN(c4ccc(C)nn4)CC3)n(C)c(=O)c2c1] C₂₉H₃₂N₆O₃ | 551 | | ES+ 513.4 [M + H] | ¹H NMR: (400 MHz, Methanol-d₄) δ 8.01 (s, 1H), 7.97-7.86 (m, 1H), 7.57 (d, J = 1.9 Hz, 1H), 7.39 (d, J = 9.4 Hz, 1H), 7.31 (d, J = 9.4 Hz, 1H), 7.20-7.08 (m, 1H), 6.58 (s, 1H), 6.57- 6.48 (m, 1H), 6.40 (d, J = 8.4 Hz, 1H), 5.19-5.08 (m, 1H), 4.36 (s, 2H), 3.72 (s, 3H), 3.42-3.35 (m, 2H), 3.30-3.12 (m, 2H), 3.10-2.81 (m, 2H), 2.54 (s, 3H), 2.41 (s, 3H), 1.66 (d, J = 6.6 Hz, 3H). |
| 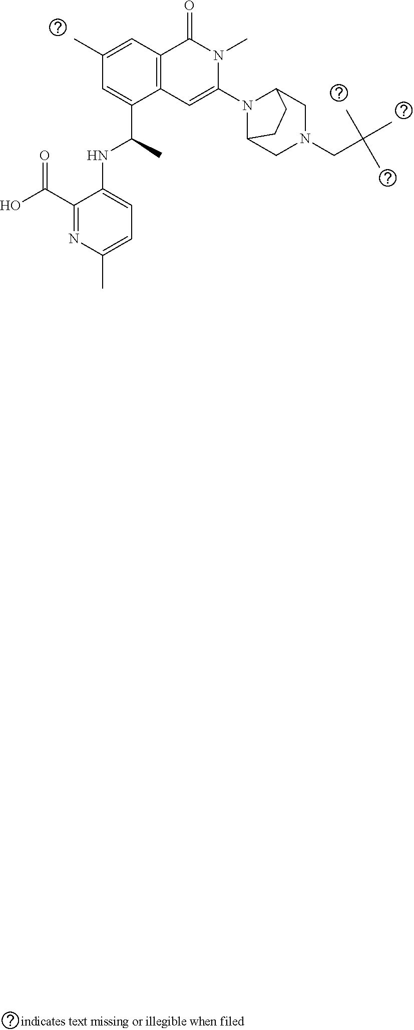 C₂₇H₂₉F₄N₅O₃ | 552 | | ES+ 548.5 [M + H] | ¹H NMR: (400 MHz, Methanol-d₄) δ 7.75 (d, J = 8.6 Hz, 1H), 7.47-7.39 (m, 1H), 7.30-6.80 (m, 2H), 6.35 (s, 1H), 5.13- 5.12 (m, 1H), 3.86 (s, 2H), 3.72 (s, 3H), 3.18-3.12 (m, 2H), 2.99-2.85 (m, 4H), 2.34 (s, 3H), 2.10- 1.96 (m, 4H), 1.67 (d, J = 6.6 Hz, 3H). |
| 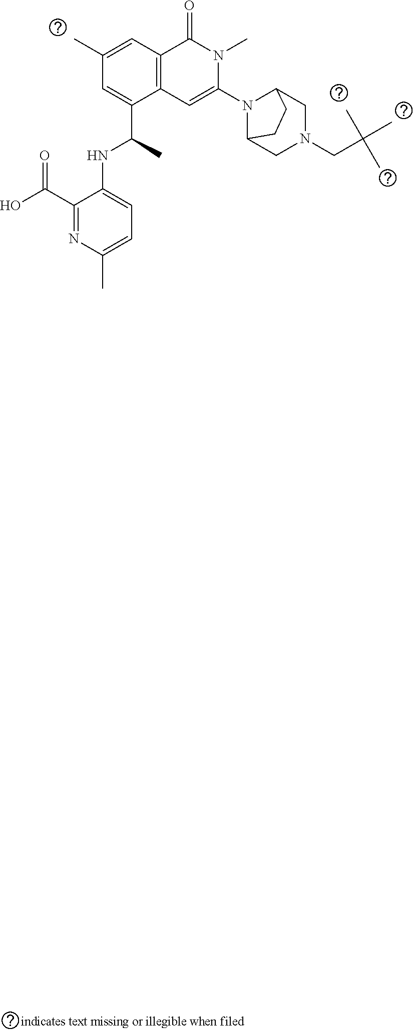 C₂₈H₂₇F₄N₅O₃ | 553 | | ES+ 558.4 [M + H] | ¹H NMR (400 MHz, Methanol-d₄) δ 7.79- 7.71 (m, 1H), 7.50- 7.41 (m, 1H), 7.22 (d, J = 8.6 Hz, 1H), 6.79 (d, J = 8.8 Hz, 1H), 6.29 (s, 1H), 5.18-5.08 (m, 1H), 3.84 (s, 2H), 3.71 (s, 3H), 3.41 (s, 1H), 3.16-3.11 (m, 2H), 2.97-2.85 (m, 4H), 2.07-1.93 (m, 4H), 1.68 (d, J = 6.6 Hz, 3H). |
| 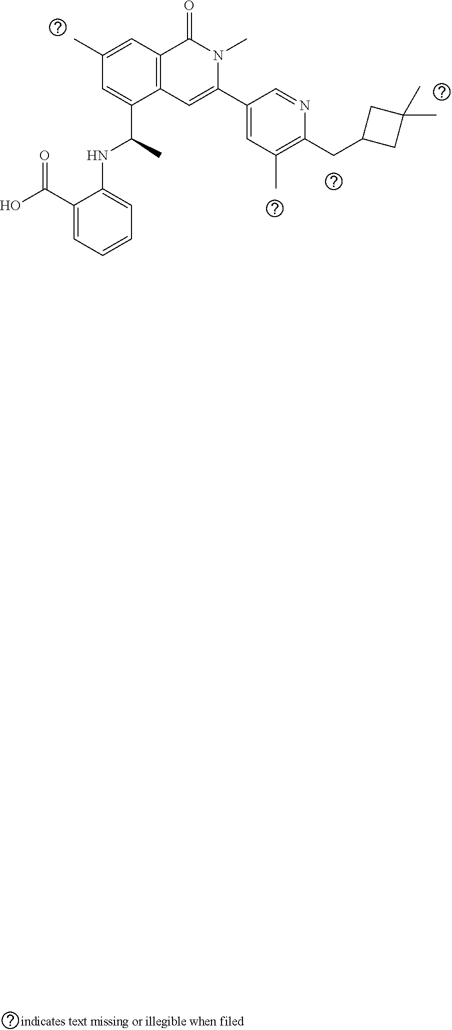 [SMILES: Cc1cc([C@@H](C)Nc2ccccc2C(=O)O)c2cc(-c3cnc(CC4CC(C)(C)C4)c(C)c3)n(C)c(=O)c2c1] C₂₈H₂₃F₄N₃O₄ | 554 | | ES− 540.05 [M − H] | ¹H NMR (400 MHz, Methanol-d₄) δ 8.15 (d, J = 2.1 Hz, 1H), 7.97-7.92 (m, 2H), 7.88-7.80 (m, 1H), 7.54-7.49 (m, 1H), 7.19-7.10 (m, 1H), 7.02 (d, J = 2.0 Hz, 1H), 6.60-6.54 (m, 1H), 6.30 (d, J = 8.5 Hz, 1H), 5.32-5.31 (m, 1H), 5.21 (d, J = 6.9 Hz, 1H), 3.49 (s, 3H), 3.28-3.16 (m, 2H), 2.92-2.76 (m, 2H), 1.67-1.60 (m, 3H). |
| 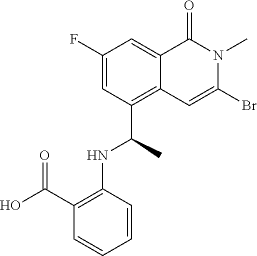 [SMILES: C[C@@H](Nc1ccccc1C(=O)O)c1cc(F)cc2c(=O)n(C)c(Br)cc12] C₁₉H₁₆BrFN₂O₃ | 555 | | ES− 417.1 [M − H] | ¹H NMR (400 MHz, DMSO-d₆) δ 12.85 (s, 1H), 8.42 (s, 1H), 7.83-7.75 (m, 2H), 7.50-7.40 (m, 2H), 7.38-7.09 (m, 1H), 6.66-6.45 (m, 1H), 6.29 (d, J = 8.4 Hz, 1H), 5.28-5.27 (m, 1H), 3.73 (s, 3H), 1.52 (d, J = 6.5 Hz, 3H). |
| 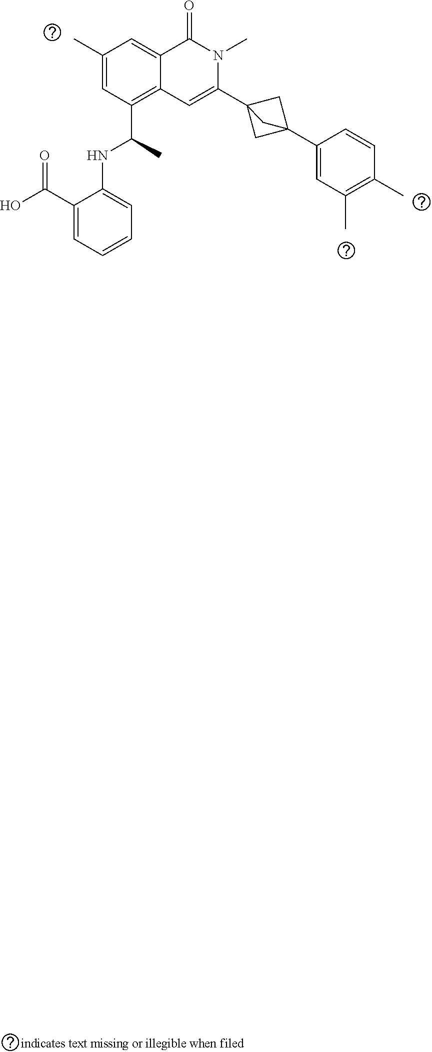 [SMILES: Cc1cc([C@@H](C)Nc2ccccc2C(=O)O)c2cc(C34CC(c5ccc(C)c(C)c5)(C3)C4)n(C)c(=O)c2c1] C₃₀H₂₅F₃N₂O₃ | 556 | | ES− 517.1 [M − H] | ¹H NMR (400 MHz, DMSO-d₆) δ 12.83 (s, 1H), 8.40 (d, J = 5.6 Hz, 1H), 7.86- 7.74 (m, 2H), 7.52- 7.34 (m, 3H), 7.26- 7.19 (m, 1H), 7.18- 7.12 (m, 1H), 6.72 (s, 1H), 6.59-6.53 (m, 1H), 6.37 (d, J = 8.5 Hz, 1H), 5.29- 5.20 (m, 1H), 3.67 (s, 3H), 2.53 (s, 6H), 1.60 (d, J = 6.6 Hz, 3H). |
| 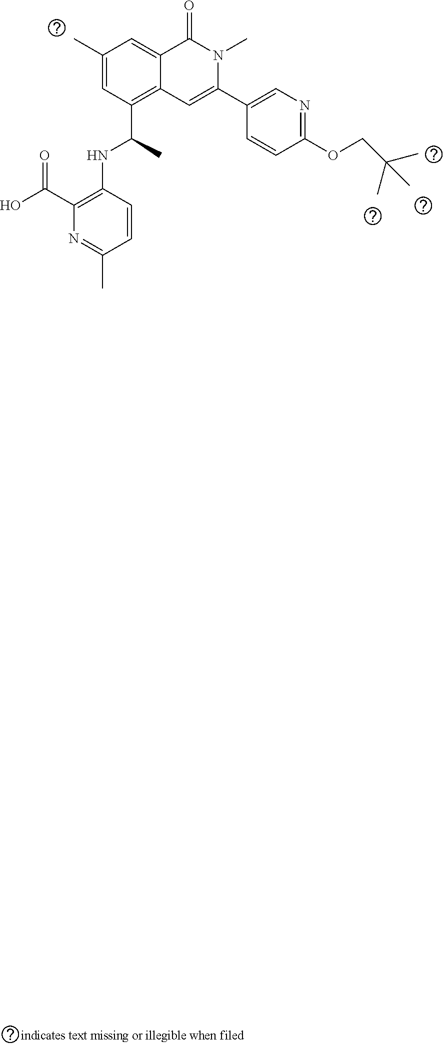 C₂₆H₂₂F₄N₄O₄ | 557 | | ES+ 531.2 [M + H] | ¹H NMR (400 MHz, DMSO-d₆) δ 8.52- 8.42 (m, 2H), 8.14- 8.08 (m, 1H), 7.87- 7.79 (m, 1H), 7.48- 7.41 (m, 1H), 7.24- 7.13 (m, 2H), 6.97 (s, 1H), 6.87 (d, J = 8.8 Hz, 1H), 5.41- 5.33 (m, 1H), 5.13- 5.05 (m, 2H), 3.36 (s, 3H), 2.35 (s, 3H), 1.54 (d, J = 6.5 Hz, 3H). |
| 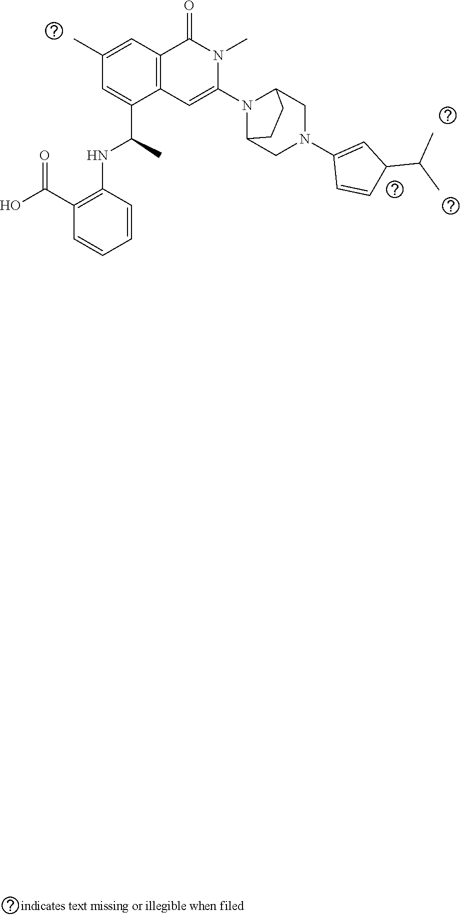 C₂₉H₂₉F₃N₆O₃ | 558 | | ES+ 567.3 [M + H] | ¹H NMR (400 MHz, DMSO-d₆) δ 12.80 (s, 1H), 8.35 (d, J = 6.0 Hz, 1H), 7.86- 7.79 (m, 1H), 7.73- 7.49 (m, 4H), 7.41- 7.33 (m, 1H), 7.26- 7.18 (m, 1H), 6.60- 6.52 (m, 1H), 6.41 (d, J = 8.5 Hz, 1H), 6.24 (s, 1H), 5.29- 5.21 (m, 1H), 4.12- 3.96 (m, 2H), 3.61 (s, 3H), 3.33-3.26 (m, 2H), 3.01-2.94 (m, 2H), 2.15-2.03 (m, 1H), 2.01-1.81 (m, 3H), 1.56 (d, J = 6.5 Hz, 3H). |
| 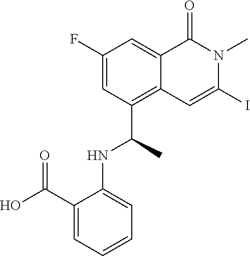 [SMILES: C[C@@H](Nc1ccccc1C(=O)O)c1cc(F)cc2c(=O)n(C)c(I)cc12] C₁₉H₁₆FIN₂O₃ | 559 | | ES− 464.8 [M − H] | ¹H NMR (400 MHz, DMSO-d₆) δ 10.27 (s, 1H), 7.80-7.76 (m, 1H), 7.70-7.66 (m, 2H), 7.46-7.43 (s, 1H), 6.87-6.79 (m, 1H), 6.38-6.30 (m, 1H), 6.05 (d, J = 8.1 Hz, 1H), 5.13- 5.06 (m, 1H), 3.77 (s, 3H), 1.44 (d, J = 6.5 Hz, 3H). |
| 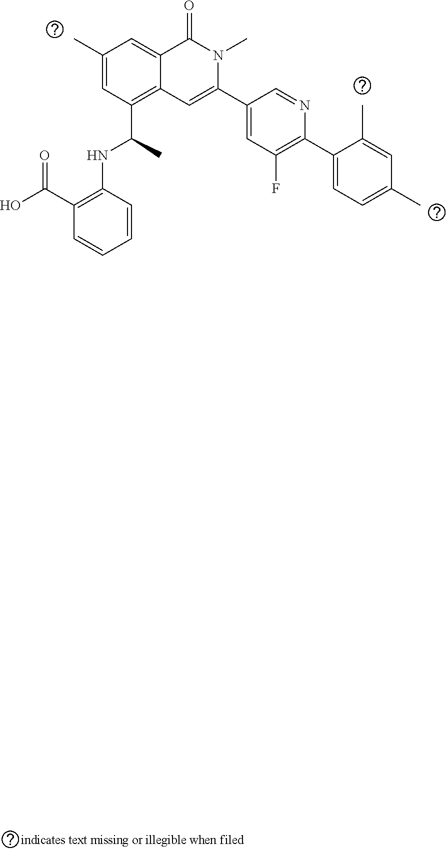 [SMILES: Cc1ccc(-c2ncc(-c3cc4c([C@@H](C)Nc5ccccc5C(=O)O)cc(C)cc4c(=O)n3C)cc2F)c(C)c1] C₃₀H₂₁F₄N₃O₃ | 560 | | ES− 546.1 [M − H] | ¹H NMR (400 MHz, Methanol-d₄) δ 8.76 (s, 1H), 8.08-8.05 (m, 1H), 7.95-7.93 (m, 2H), 7.79-7.73 (m, 1H), 7.55-7.52 (m, 1H), 7.23-7.14 (m, 4H), 6.60-6.56 (m, 1H), 6.34 (d, J = 8.5 Hz, 1H), 5.28- 5.26 (m, 1H), 3.56 (s, 3H), 1.66 (d, J = 6.7 Hz, 3H). |
| 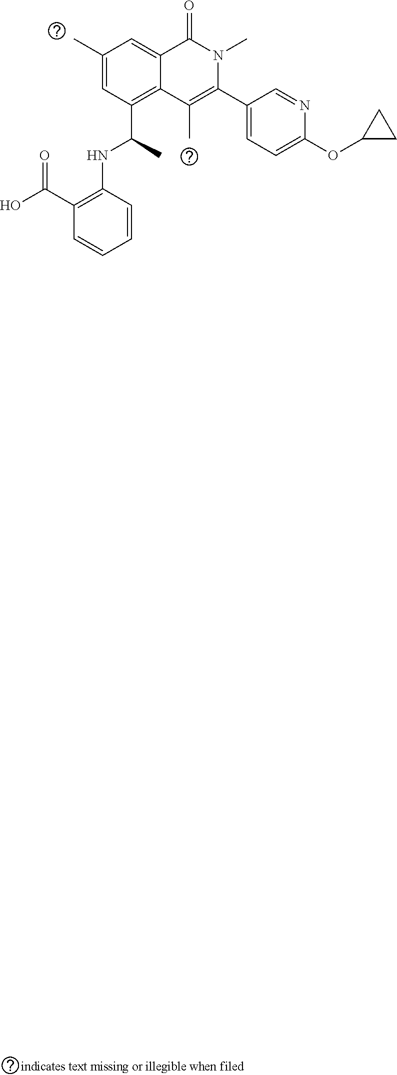 C₂₇H₂₃F₂N₃O₄ | 561 | | ES+ 492.4 [M + H] | ¹H NMR (400 MHz, DMSO-d₆) δ 12.87 (s, 1H), 8.47-8.45 (m, 2H), 8.10-7.94 (m, 2H), 7.90-7.81 (m, 1H), 7.61 (d, J = 9.9 Hz, 1H), 7.28- 7.18 (m, 1H), 7.09 (d, J = 8.6 Hz, 1H), 6.63-6.55 (m, 1H), 6.30 (d, J = 8.4 Hz, 1H), 5.37-5.27 (m, 1H), 4.36-4.28 (m, 1H), 3.28 (s, 3H), 1.57 (d, J = 6.4 Hz, 3H), 0.87-0.70 (m, 4H). |
| 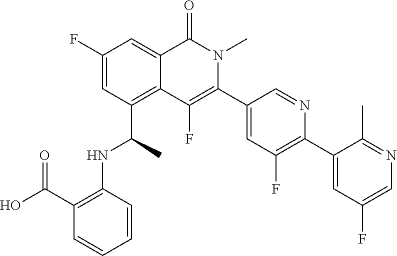 [SMILES: Cc1ncc(F)cc1-c1ncc(-c2c(F)c3c([C@@H](C)Nc4ccccc4C(=O)O)cc(F)cc3c(=O)n2C)cc1F] C₃₀H₂₂F₄N₄O₃ | 562 | | ES+ 563.1 [M + H] | ¹H NMR (400 MHz, DMSO-d₆) δ 12.88 (s, 1H), 8.89 (s, 1H), 8.65 (d, J = 3.0 Hz, 1H), 8.48 (s, 1H), 8.39 (d, J = 10.3 Hz, 1H), 8.02 (d, J = 8.4 Hz, 1H), 7.94-7.92 (m, 1H), 7.86 (d, J = 7.8 Hz, 1H), 7.64 (d, J = 10.0 Hz, 1H), 7.21-7.19 (m, 1H), 6.59-6.52 (m, 1H), 6.32 (d, J = 7.2 Hz, 1H), 5.35-5.33 (m, 1H), 3.36 (s, 3H), 2.51 (s, 3H), 1.60 (d, J = 6.5 Hz, 3H). |
| 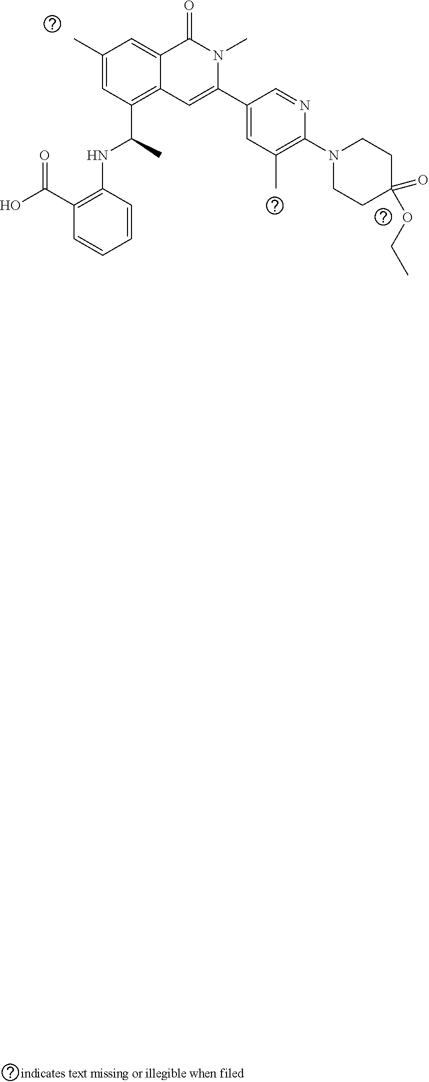 C₃₀H₃₁F₂N₄O₅P | 563 | | ES+ 597.5 [M + H] | ¹H NMR (400 MHz, DMSO-d₆) δ 12.82 (s, 1H), 8.51 (s, 1H), 8.30-8.28 (m, 1H), 7.95-7.92 (m, 1H), 7.86-7.79 (m, 2H), 7.45-7.42 (m, 1H), 7.21-7.12 (m, 1H), 7.00 (s, 1H), 6.57- 6.54 (m, 1H), 6.30 (d, J = 8.5 Hz, 1H), 5.31-5.30 (m, 1H), 4.36-4.21 (m, 2H), 4.08-4.02 (m, 2H), 3.54-3.51 (m, 2H), 3.48-3.42 (m, 3H), 2.08-1.95 (m, 4H), 1.53 (d, J = 6.6 Hz, 3H), 1.33-1.30 (m, 3H). |
| 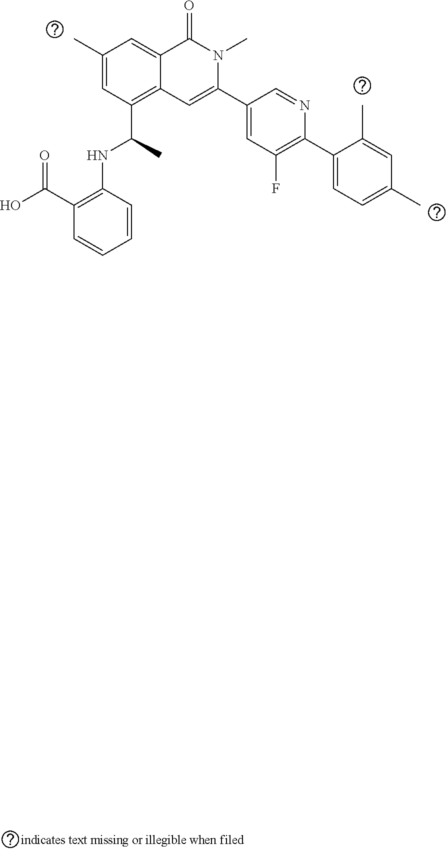 [SMILES: Cc1ccc(-c2ncc(-c3cc4c([C@@H](C)Nc5ccccc5C(=O)O)cc(C)cc4c(=O)n3C)cc2F)c(C)c1] C₃₀H₂₁ClF₃N₃O₃ | 564 | | ES− 562.0 [M − H] | ¹H NMR (400 MHz, Methanol-d₄) δ 8.77- 8.72 (m, 1H), 8.12- 8.04 (m, 1H), 7.96- 7.89 (m, 2H), 7.67- 7.60 (m, 1H), 7.60- 7.53 (m, 1H), 7.50- 7.43 (m, 1H),7.36- 7.26 (m, 1H), 7.16 (s, 1H), 7.14-7.05 (m, 1H), 6.60-6.51 (m, 1H), 6.29 (d, J = 8.3 Hz, 1H), 5.30- 5.20 (m, 1H), 3.56 (s, 3H), 1.65 (d, J = 6.7 Hz, 3H). |
| 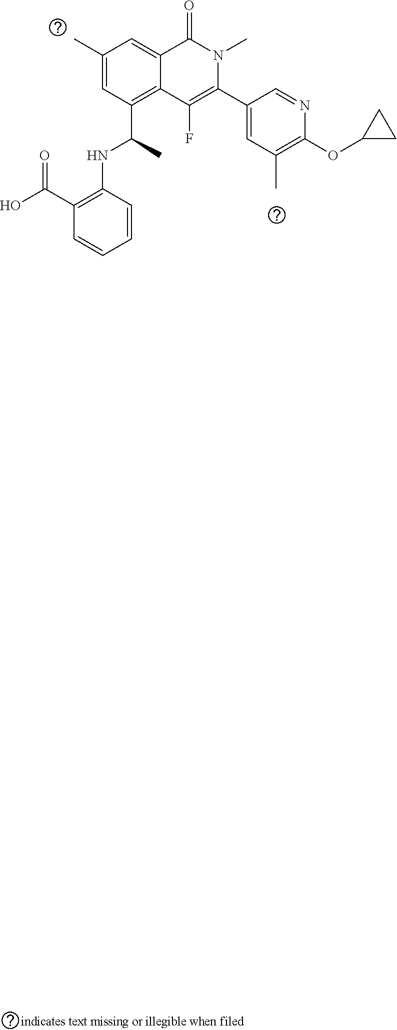 [SMILES: Cc1cc([C@@H](C)Nc2ccccc2C(=O)O)c2c(F)c(-c3cnc(OC4CC4)c(C)c3)n(C)c(=O)c2c1] C₂₇H₂₂F₃N₃O₄ | 565 | | ES− 508.4 [M − H] | ¹H NMR (400 MHz, DMSO-d₆) δ 12.87 (s, 1H), 8.45 (s, 1H), 8.33 (s, 1H), 8.11- 8.08 (m, 1H), 7.98- 7.86 (m, 1H), 7.85- 7.84 (m, 1H), 7.62 (d, J = 9.9 Hz, 1H), 7.25-7.16 (m, 1H), 6.59-6.32 (m, 1H), 6.30 (s, 1H), 5.32- 5.30 (m, 1H), 4.46- 4.44 (m, 1H), 3.30 (s, 3H), 1.57 (d, J = 6.5 Hz, 3H), 0.84- 0.82 (m, 4H). |
| 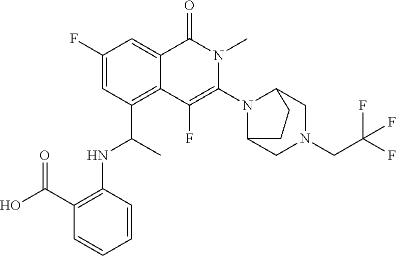 [SMILES: CC(Nc1ccccc1C(=O)O)c1cc(F)cc2c(=O)n(C)c(N3C4CCC3CN(CC(F)(F)F)C4)c(F)c12] C₂₇H₂₇F₅N₄O₃ | 566 | enantiomer 1 | ES+ 551.2 [M + H] | ¹H NMR (400 MHz, DMSO-d₆) δ 12.81 (s, 1H), 8.46 (s, 1H), 7.89-7.72 (m, 2H), 7.48 (s, 1H), 7.25- 7.16 (m, 1H), 6.57 (d, J = 7.5 Hz, 1H), 6.30 (d, J = 8.3 Hz, 1H), 5.28-5.26 (m, 1H), 3.96-3.94 (m, 2H), 3.62 (s, 3H), 3.26-3.24 (m, 2H), 2.95-2.93 (m, 2H), 2.82-2.80 (m, 2H), 1.97-1.96 (m, 4H), 1.56 (d, J = 6.4 Hz, 3H). |
| 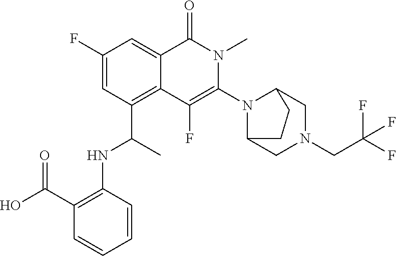 [SMILES: CC(Nc1ccccc1C(=O)O)c1cc(F)cc2c(=O)n(C)c(N3C4CCC3CN(CC(F)(F)F)C4)c(F)c12] C₂₇H₂₇F₅N₄O₃ | 567 | enantiomer 2 | ES+ 551.2 [M + H] | ¹H NMR (400 MHz, DMSO-d₆) δ 12.83 (s, 1H), 8.44 (s, 1H), 7.86-7.76 (m, 2H), 7.49 (s, 1H), 7.20- 7.10 (m, 1H), 6.57 (d, J = 7.5 Hz, 1H), 6.30 (d, J = 8.5 Hz, 1H), 5.28-5.27 (m, 1H), 3.96-3.93 (m, 2H), 3.62 (s, 3H), 3.27-3.25 (m, 2H), 2.94-2.91 (m, 2H), 2.82-2.80 (m, 2H), 1.96-1.94 (m, 4H), 1.56 (d, J = 6.5 Hz, 3H) |
| 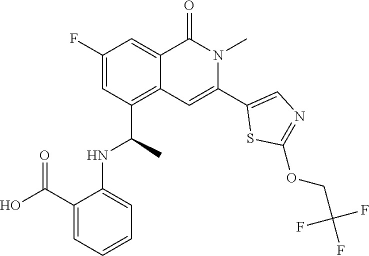 [SMILES: C[C@@H](Nc1ccccc1C(=O)O)c1cc(F)cc2c(=O)n(C)c(-c3cnc(OCC(F)(F)F)s3)cc12] C₂₄H₁₉F₄N₃O₄S | 568 | | ES+ 522.1 [M + H] | ¹H NMR (400 MHz, CD₃CN) δ 8.41-8.27 (m, 1H), 7.92-7.84 (m, 2H), 7.48-7.41 (m, 1H), 7.37 (s, 1H), 7.19-7.11 (m, 1H), 7.03 (s, 1H), 6.63- 6.51 (m, 1H), 6.28 (d, J = 8.4 Hz, 1H), 5.23-5.10 (m, 1H), 5.05-4.95 (m, 2H), 3.51 (s, 3H), 1.58 (d, J = 6.8 Hz, 3H). |
| 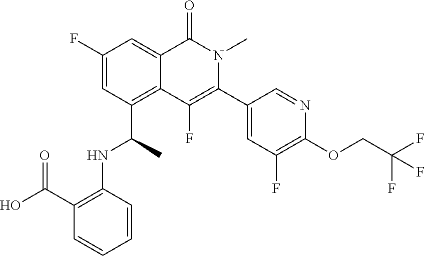 [SMILES: C[C@@H](Nc1ccccc1C(=O)O)c1cc(F)cc2c(=O)n(C)c(-c3cnc(OCC(F)(F)F)c(F)c3)c(F)c12] C₂₆H₁₉F₆N₃O₄ | 569 | | ES+ 552.1 [M + H] | ¹H NMR: (400 MHz, Methanol-d₄) δ 8.22 (s, 1H), 8.03-8.00 (m, 1H), 7.96-7.94 (m, 2H), 7.70-7.67 (m, 1H), 7.18-7.14 (m, 1H), 6.61-6.57 (m, 1H), 6.31 (d, J = 8.4 Hz, 1H), 5.43- 5.41 (m, 1H), 5.12- 5.05 (m, 2H), 3.41 (s, 3H), 1.63-1.62 (m, 3H). |
| 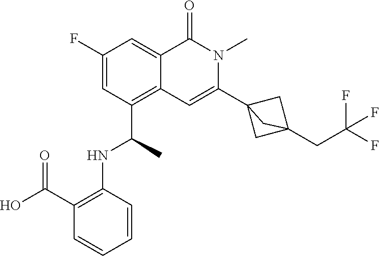 [SMILES: C[C@@H](Nc1ccccc1C(=O)O)c1cc(F)cc2c(=O)n(C)c(C34CC(CC(F)(F)F)(C3)C4)cc12] C₂₆H₂₄F₄N₂O₃ | 570 | | ES+ 489.1 [M + H] | ¹H NMR (DMSO-d₆, 400 MHz) 12.81 (s, 1H), 8.37 (s, 1H), 7.86-7.80 (m, 1H), 7.79-7.73 (m, 1H), 7.49-7.41 (m, 1H), 7.25-7.15 (m, 1H), 6.64 (s, 1H), 6.57- 6.52 (m, 1H), 6.37 (d, J = 8.4 Hz, 1H), 5.28-5.26 (m, 1H), 3.62 (s, 3H), 2.68- 2.62 (m, 2H), 2.28- 2.26 (m, 6H), 1.57 (d, J = 6.6 Hz, 3H). |
| 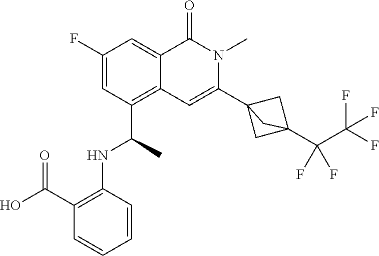 [SMILES: C[C@@H](Nc1ccccc1C(=O)O)c1cc(F)cc2c(=O)n(C)c(C34CC(C(F)(F)C(F)(F)F)(C3)C4)cc12] C₂₆H₂₂F₆N₂O₃ | 571 | | ES+ 525.4 [M + H] | ¹H NMR (400 MHz, DMSO-d₆) δ 12.82 (s, 1H), 8.44 (s, 1H), 7.86-7.74 (m, 2H), 7.55-7.30 (m, 1H), 7.25-7.16 (m, 1H), 6.71 (s, 1H), 6.57- 6.54 (m, 1H), 6.36 (d, J = 8.4 Hz, 1H), 5.31-5.30 (m, 1H), 3.63 (s, 3H), 2.51- 2.50 (m, 6H), 1.57 (d, J = 6.6 Hz, 3H). |
| 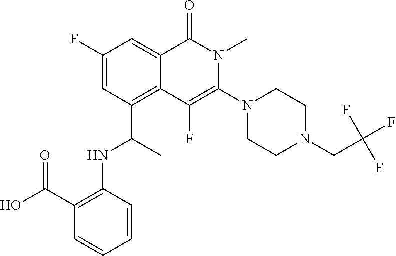 [SMILES: CC(Nc1ccccc1C(=O)O)c1cc(F)cc2c(=O)n(C)c(N3CCN(CC(F)(F)F)CC3)c(F)c12] C₂₅H₂₅F₅N₄O₃ | 572 | enantiomer 1 | ES+ 525.2 [M + H] | ¹H NMR (400 MHz, Methanol-d₄) δ 7.96- 7.90 (m, 2H), 7.61- 7.59 (m, 1H), 7.19- 7.15 (m, 1H), 6.65- 6.58 (m, 1H), 6.31 (d, J = 8.5 Hz, 1H), 5.47-5.39 (m, 1H), 3.68 (s, 3H), 3.56- 3.47 (m, 2H), 3.26- 3.15 (m, 4H), 3.03- 2.79 (m, 4H), 1.65 (d, J = 6.5 Hz, 3H). |
| 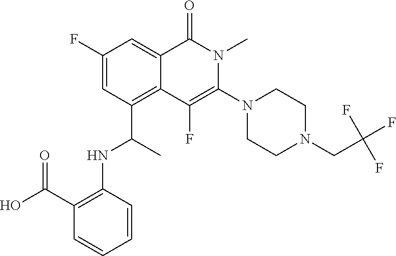 [SMILES: CC(Nc1ccccc1C(=O)O)c1cc(F)cc2c(=O)n(C)c(N3CCN(CC(F)(F)F)CC3)c(F)c12] C₂₅H₂₅F₅N₄O₃ | 573 | enantiomer 2 | ES+ 525.4 [M + H] | ¹H NMR (400 MHz, Methanol-d₄) δ 7.96- 7.90 (m, 2H), 7.62- 7.60 (m, 1H), 7.20- 7.15 (m, 1H), 6.64- 6.57 (m, 1H), 6.30 (d, J = 8.5 Hz, 1H), 5.46-5.44 (m, 1H), 3.68 (s, 3H), 3.56- 3.46 (m, 2H), 3.26- 3.18 (m, 4H), 3.03- 2.73 (m, 4H), 1.64 (d, J = 6.5 Hz, 3H). |
| 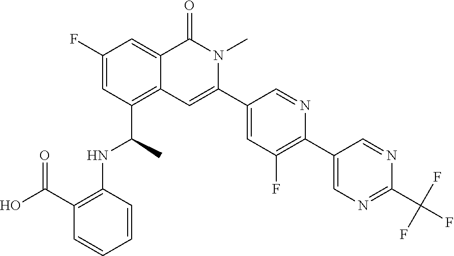 [SMILES: C[C@@H](Nc1ccccc1C(=O)O)c1cc(F)cc2c(=O)n(C)c(-c3cnc(-c4cnc(C(F)(F)F)nc4)c(F)c3)cc12] C₂₉H₂₀F₅N₅O₃ | 574 | | ES+ 582.1 [M + H] | ¹H NMR (400 MHz, DMSO-d₆) δ 12.80 (s, 1H), 9.64 (s, 2H), 8.99 (d, J = 1.8 Hz, 1H), 8.69 (s, 1H), 8.48-8.41 (m, 1H), 7.90-7.79 (m, 2H), 7.46-7.45 (m, 1H), 7.17 (s, 1H), 7.13 (s, 1H), 6.57-6.48 (m, 1H), 6.30 (d, J = 8.5 Hz, 1H), 5.34 (s, 1H), 3.46 (s, 3H), 1.54 (d, J = 6.5 Hz, 3H). |
| 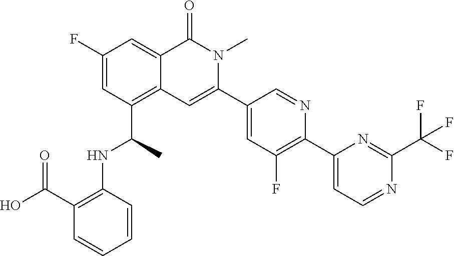 [SMILES: C[C@@H](Nc1ccccc1C(=O)O)c1cc(F)cc2c(=O)n(C)c(-c3cnc(-c4ccnc(C(F)(F)F)n4)c(F)c3)cc12] C₂₉H₂₀F₅N₅O₃ | 575 | | ES− 580.1 [M − H] | ¹H NMR (400 MHz, DMSO-d₆) δ 12.83 (s, 1H), 9.49-9.44 (m, 1H), 9.30 (d, J = 5.3 Hz, 1H), 8.80- 8.74 (m, 1H), 8.66 (d, J = 5.3 Hz, 1H), 8.43 (s, 1H), 7.92- 7.87 (m, 1H), 7.84- 7.80 (m, 1H), 7.53- 7.47 (m, 1H), 7.35 (s, 1H), 7.22-7.15 (m, 1H), 6.59-6.51 (m, 1H), 6.29 (d, J = 8.5 Hz, 1H), 5.28 (s, 1H), 3.42 (s, 3H), 1.55 (d, J = 6.5 Hz, 3H). |
| 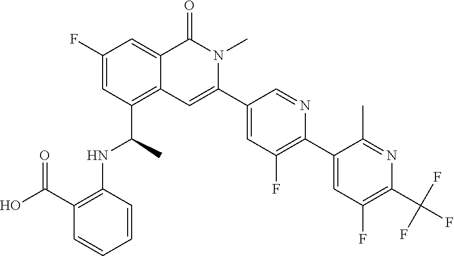 [SMILES: Cc1nc(C(F)(F)F)c(F)cc1-c1ncc(-c2cc3c([C@@H](C)Nc4ccccc4C(=O)O)cc(F)cc3c(=O)n2C)cc1F] C₃₁H₂₂F₆N₄O₃ | 576 | | ES− 611.1 [M − H] | ¹H NMR (400 MHz, DMSO-d₆) δ 8.94- 8.88 (m, 1H), 8.54 (s, 1H), 8.43-8.35 (m, 1H), 8.28 (d, J = 10.6 Hz, 1H), 7.91- 7.79 (m, 2H), 7.50- 7.42 (m, 1H), 7.16 (s, 2H), 6.58-6.50 (m, 1H), 6.32 (d, J = 8.5 Hz, 1H), 5.36 (s, 1H), 3.47 (s, 3H), 2.54 (s, 3H), 1.56 (d, J = 6.5 Hz, 3H). |
| 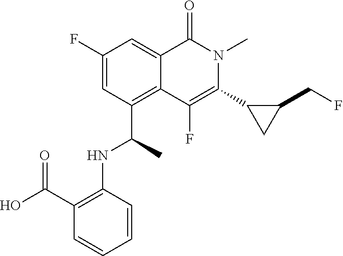 C₂₃H₂₁F₃N₂O₃ | 577 | trans cyclopropyl diastereomer 1 | ES− 429.2 [M − H] | ¹H NMR (400 MHz, Methanol-d₄) δ 8.00- 7.86 (m, 2H), 7.65- 7.57 m, 1H), 7.18- 7.10 (m, 1H), 6.62- 6.53 (m, 1H), 6.28 (d, J = 8.5 Hz, 1H), 5.51-5.42 (m, 1H), 4.76-4.57 (m, 1H), 4.56-4.36 (m, 1H), 3.80 (s, 3H), 2.03- 1.97 (m, 1H), 1.90- 1.80 (m, 1H), 1.63 (d, J = 8.0 Hz, 3H), 1.37-1.25 (m, 2H). |
| 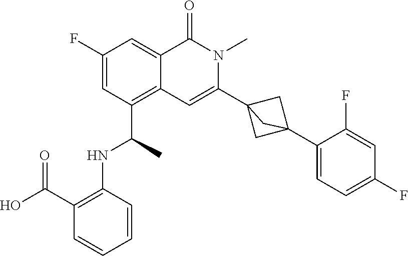 [SMILES: C[C@@H](Nc1ccccc1C(=O)O)c1cc(F)cc2c(=O)n(C)c(C34CC(c5ccc(F)cc5F)(C3)C4)cc12] C₃₀H₂₅F₃N₂O₃ | 578 | | ES− 517.2 [M − H] | ¹H NMR (400 MHz, Methanol-d₄) δ 7.94- 7.92 (m, 1H), 7.85- 7.82 (m, 1H), 7.50- 7.47 (m, 1H), 7.33- 7.27 (m, 1H), 7.20- 7.16 (m, 1H), 6.99- 6.88 (m, 2H), 6.84 (d, J = 2.9 Hz, 1H), 6.59-6.56 (m, 1H), 6.38-6.36 (m, 1H), 5.24-5.17 (m, 1H), 3.80 (s, 3H), 2.63- 2.57 (m, 6H), 1.68 (d, J = 3.6 Hz, 3H). |
| 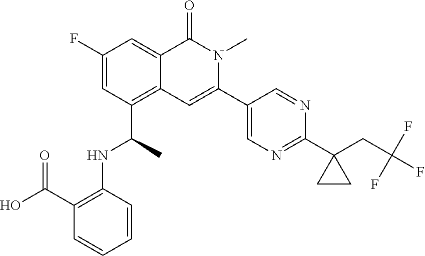 [SMILES: C[C@@H](Nc1ccccc1C(=O)O)c1cc(F)cc2c(=O)n(C)c(-c3cnc(C4(CC(F)(F)F)CC4)nc3)cc12] C₂₈H₂₄F₄N₄O₃ | 579 | | ES+ 541.1 [M + H] | ¹H NMR: (400 MHz, CD₃CN) δ 8.80 (s, 2H), 8.46-8.24 (m, 1H), 7.96-7.87 (m, 2H), 7.48-7.47 (m, 1H), 7.20-7.10 (m, 1H), 6.85 (s, 1H), 6.57 (t, J = 7.6 Hz, 1H), 6.28 (d, J = 8.4 Hz, 1H), 5.22-5.10 (m, 1H), 3.40 (s, 3H), 3.09-3.01 (m, 2H), 1.58 (d, J = 6.8 Hz, 3H), 1.55-1.51 (m, 2H), 1.29-1.22 (m, 2H) |
| 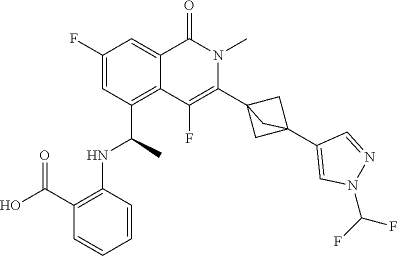 [SMILES: C[C@@H](Nc1ccccc1C(=O)O)c1cc(F)cc2c(=O)n(C)c(C34CC(c5cnn(C(F)F)c5)(C3)C4)c(F)c12] C₂₈H₂₄F₄N₄O₃ | 580 | | ES+ 541.3 [M + H] | ¹H NMR (400 MHz, DMSO-d₆) δ 8.54 (s, 1H), 8.16 (s, 1H), 7.94-7.60 (m, 4H), 7.57 (d, J = 9.7 Hz, 1H), 7.24-7.15 (m, 1H), 6.58-6.58 (m, 1H), 6.24 (d, J = 8.6 Hz, 1H), 5.31-5.22 (m, 1H), 3.67 (s, 3H), 2.70 (d, J = 1.5 Hz, 6H), 1.57 (d, J = 6.5 Hz, 3H). |
| 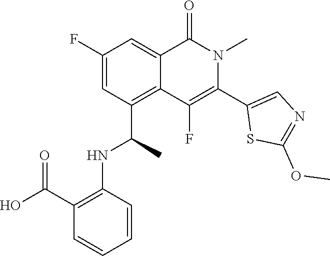 [SMILES: COc1ncc(-c2c(F)c3c([C@@H](C)Nc4ccccc4C(=O)O)cc(F)cc3c(=O)n2C)s1] C₂₃H₁₉F₂N₃O₄S | 581 | | ES− 470.0 [M − H] | ¹H NMR (400 MHz, Chloroform-d) δ 8.20-8.02 (m, 3H), 7.69-7.65 (m, 1H), 7.35-7.21 (m, 2H), 6.65-6.61 (m, 1H), 6.28 (d, J = 8.6 Hz, 1H), 5.40-5.37 (m, 1H), 4.18 (s, 3H), 3.53 (s, 3H), 1.67- 1.61 (m, 3H). |
| 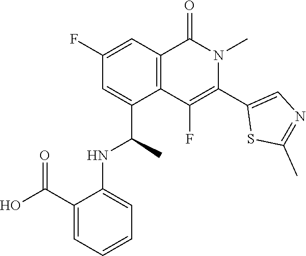 [SMILES: Cc1ncc(-c2c(F)c3c([C@@H](C)Nc4ccccc4C(=O)O)cc(F)cc3c(=O)n2C)s1] C₂₃H₁₉F₂N₃O₃S | 582 | | ES+ 456.0 [M + H] | ¹H NMR (400 MHz, DMSO-d₆) δ 12.85 (s, 1H), 8.43 (s, 1H), 7.98-7.93 (m, 2H), 7.85-7.82 (m, 1H), 7.61 (d, J = 9.8 Hz, 1H), 7.22-7.18 (m, 1H), 6.62-6.54 (m, 1H), 6.28 (d, J = 8.5 Hz, 1H), 5.33-5.26 (m, 1H), 3.31 (s, 3H), 2.77 (s, 3H), 1.56 (d, J = 6.4 Hz, 3H). |
| 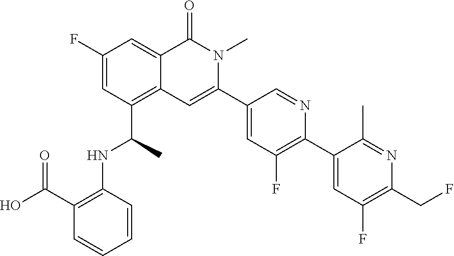 [SMILES: Cc1nc(CF)c(F)cc1-c1ncc(-c2cc3c([C@@H](C)Nc4ccccc4C(=O)O)cc(F)cc3c(=O)n2C)cc1F] C₃₁H₂₄F₄N₄O₃ | 583 | | ES+ 577.4 [M + H] | ¹H NMR (400 MHz, Methanol-d₄) δ 8.83- 8.78 (m, 1H), 8.16- 8.09 (m, 1H), 7.98- 7.91 (m, 2H), 7.84 (d, J = 9.6 Hz, 1H), 7.60-7.52 (m, 1H), 7.20-7.10 (m, 2H), 6.62-6.53 (m, 1H), 6.33 (d, J = 8.4 Hz, 1H), 5.66 (d, J = 2.1 Hz, 1H), 5.54 (d, J = 2.1 Hz, 1H), 5.31- 5.22 (m, 1H), 3.57 (s, 3H), 2.55 (s, 3H), 1.66 (d, J = 6.7 Hz, 3H) |
| 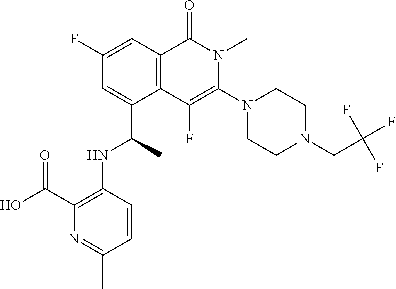 [SMILES: Cc1ccc(N[C@H](C)c2cc(F)cc3c(=O)n(C)c(N4CCN(CC(F)(F)F)CC4)c(F)c23)c(C(=O)O)n1] C₂₅H₂₆F₅N₅O₃ | 584 | | ES+ 540.2 [M + H] | ¹H NMR (400 MHz, DMSO-d₆) δ 9.79 (s, 1H), 7.79 (d, J = 8.9 Hz, 1H), 7.43 (s, 1H), 7.18-6.92 (m, 1H), 6.85-6.68 (m, 1H), 5.29 (s, 1H), 3.52 (s, 3H), 3.35-3.26 (m, 4H), 3.16 (d, J = 14.3 Hz, 2H), 2.93 (d, J = 10.8 Hz, 2H), 2.73- 2.62 (m, 2H), 2.34 (d, J = 6.6 Hz, 1H), 2.15 (d, J = 26.7 Hz, 2H), 1.55 (d, J = 6.8 Hz, 3H). |
| 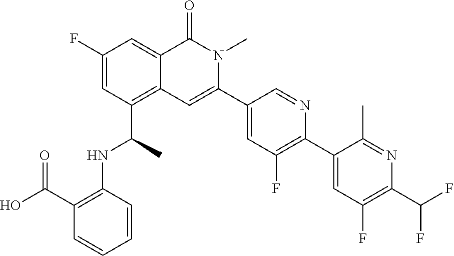 [SMILES: Cc1nc(C(F)F)c(F)cc1-c1ncc(-c2cc3c([C@@H](C)Nc4ccccc4C(=O)O)cc(F)cc3c(=O)n2C)cc1F] C₃₁H₂₃F₅N₄O₃ | 585 | | | |
| 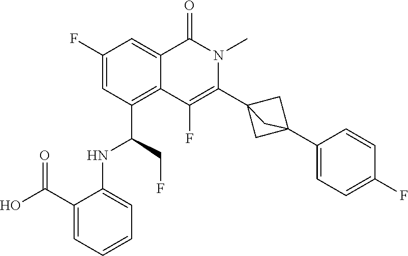 [SMILES: Cn1c(C23CC(c4ccc(F)cc4)(C2)C3)c(F)c2c([C@@H](CF)Nc3ccccc3C(=O)O)cc(F)cc2c1=O] C₃₀H₂₄F₄N₂O₃ | 586 | | | |
| 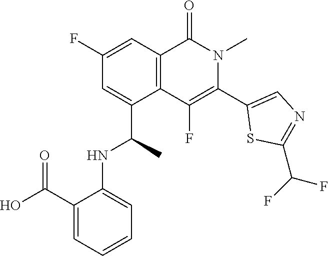 [SMILES: C[C@@H](Nc1ccccc1C(=O)O)c1cc(F)cc2c(=O)n(C)c(-c3cnc(C(F)F)s3)c(F)c12] C₂₃H₁₇F₄N₃O₃S | 587 | | | |
| 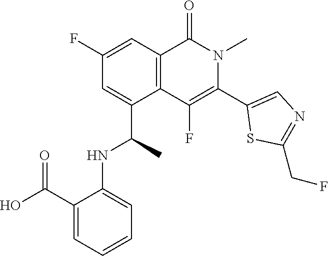 [SMILES: C[C@@H](Nc1ccccc1C(=O)O)c1cc(F)cc2c(=O)n(C)c(-c3cnc(CF)s3)c(F)c12] C₂₃H₁₈F₃N₃O₃S | 588 | | | |
| 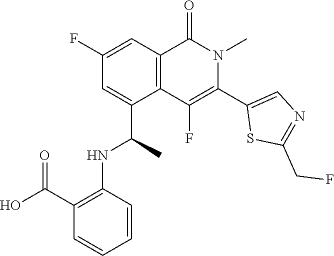 [SMILES: C[C@@H](Nc1ccccc1C(=O)O)c1cc(F)cc2c(=O)n(C)c(-c3cnc(CF)s3)c(F)c12] C₂₇H₃₀F₂N₄O₄ | 589 | | | |
| 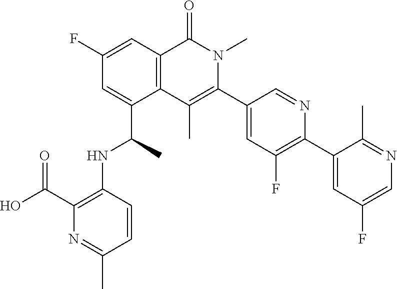 [SMILES: Cc1ccc(N[C@H](C)c2cc(F)cc3c(=O)n(C)c(-c4cnc(-c5cc(F)cnc5C)c(F)c4)c(C)c23)c(C(=O)O)n1] C₃₀H₂₃F₄N₅O₃ | 590 | | | |
| 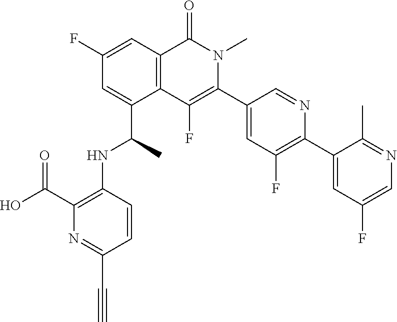 [SMILES: C#Cc1ccc(N[C@H](C)c2cc(F)cc3c(=O)n(C)c(-c4cnc(-c5cc(F)cnc5C)c(F)c4)c(F)c23)c(C(=O)O)n1] C₃₁H₂₁F₄N₅O₃ | 591 | | | |
| 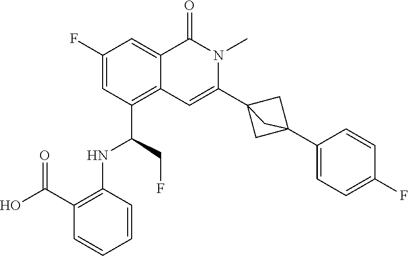 [SMILES: Cn1c(C23CC(c4ccc(F)cc4)(C2)C3)cc2c([C@@H](CF)Nc3ccccc3C(=O)O)cc(F)cc2c1=O] C₃₀H₂₅F₃N₂O₃ | 592 | | | |
| 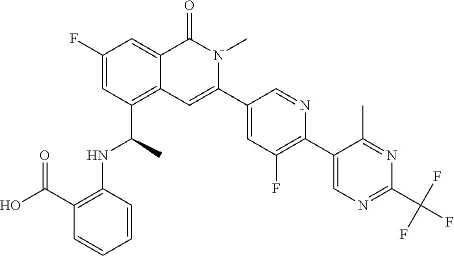 [SMILES: Cc1nc(C(F)(F)F)ncc1-c1ncc(-c2cc3c([C@@H](C)Nc4ccccc4C(=O)O)cc(F)cc3c(=O)n2C)cc1F] C₃₀H₂₂F₅N₅O₃ | 593 | | | |
| 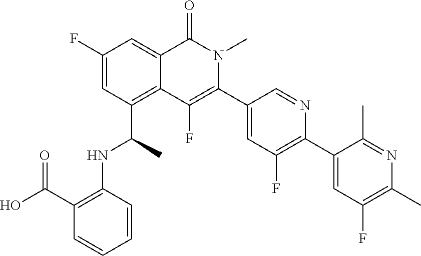 [SMILES: Cc1nc(C)c(-c2ncc(-c3c(F)c4c([C@@H](C)Nc5ccccc5C(=O)O)cc(F)cc4c(=O)n3C)cc2F)cc1F] C₃₁H₂₄F₄N₄O₃ | 594 | | | |
| 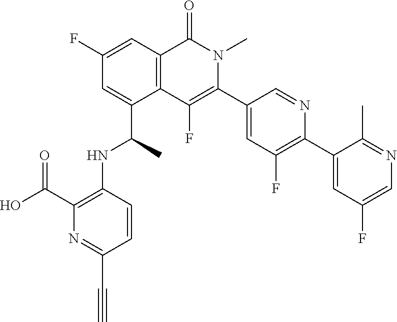 [SMILES: C#Cc1ccc(N[C@H](C)c2cc(F)cc3c(=O)n(C)c(-c4cnc(-c5cc(F)cnc5C)c(F)c4)c(F)c23)c(C(=O)O)n1] C₂₆H₂₄F₅N₅O₃ | 595 | | | |

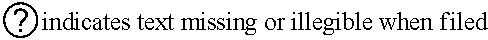

### Assays and Compound Testing

### In Vitro Cell Proliferation: Determination of EC50 Values for Inhibition of Proliferation in T47D Cells Expressing Mutant PI3Ka (H1047R) Mutation and SKBR3 Cells Express WT PI3Ka.

T47D or SKBR3 cells were trypsinized, resuspended in culture media and seeded onto assay ready plates. T47D culture media consisted of RPMI, 10% FBS and Insulin (0.2 units/mL). SKBR3 culture media consisted of McCoys 5a and 10% FBS. Cells were seeded at a density of 1,500 cells/well and dispensed in 50 μl onto 384 well assay ready plates (Corning, 89089-790). Assay ready plates had previously been stamped with 10-point dilutions of compounds of interest, as well as controls. The Echo655 is used to stamp plates at 40 nL of compound or DMSO. Cells were grown for 72 hours at 37° Celsius and 5% CO₂. After 72 hours, cells were equilibrated at room temperature for 15 minutes. 30 μL of CellTiter-Glo reagent is added to the plate, which is then shaken for 30 minutes at temperature at 300-500 rpm. Cells are then read on an Envision plate reader. The percentage of inhibition of proliferation was calculated using the following formula: % Inhibition=100×(Lum_{D}−Lumₛₐₘₚₗₑ)/(Lum_{D}−Lumₗₙₕ), where D is obtained from cells treated with 0.1% DMSO only; lnh is obtained from cells treated with 10 uM Alpelisib. The effective concentration achieving 50% inhibition of proliferation (EC50) is calculated by fitting the Curve using Xlfit (v5.3.1.3), equation 201: Y=Bottom+(Top−Bottom)/(1+10\hat{ }((LogEC50−X)*HillSlope)).

### Reagent Table:

| Reagent | Vendor | Cat No. |
|---|---|---|
| Cell Titer Glo 2.0 Assay 500 mL | Promega | G9243 |
| Corning 384-well Low Flange Black Flat Bottom | Corning | 89089-790 |
| Polystyrene TC-treated Microplates, 10 per Bag, | | |
| with Lid, Sterile SKU: 3571 | | |
| PBS | Solarbio | P1020 |
| Trypan blue solution 0.4% | Fisher | T10282 |
| Fetal Bovine Serum Australia origin | Sigma | 82051-458 |
| Gibco RPMI 1640 Medium | Fisher | 11875093 |
| Gibco McCoy's 5A (modified) Medium, HEPES | Fisher | 12330031 |
| DMEM, high glucose, HEPES, no phenol red | Fisher | 21063045 |
| Insulin, human recombinant, zinc solution | Fisher | 12585-014 |

In Vitro Cell pAKT: Determination of IC50 Values for Inhibition of Phosphorylation of AKT (pAKT) in T47D Cells Expressing Mutant PI3Ka (H1047R) Mutation and SKBR3 Cells Express WT PI3Ka.

T47D or SKBR3 cells were trypsinized, resuspended in culture media and seeded onto assay ready plates. T47D culture media consisted of RPMI, 10% FBS and Insulin (0.2 units/mL). SKBR3 culture media consisted of McCoy's 5a and 10% FBS. Cells were seeded at a density of 5000 cells/well and dispensed in 12.5 μL onto 384 well assay ready plates (Perkin Elmer, 6008238)). Assay ready plates had previously been stamped with 10-point dilutions of compounds of interest, as well as controls. The Echo655 is used to stamp plates at 12.5 nL of compound or DMSO. Cells were grown for 6 hours at 37° Celsius and 5% CO₂. After 6 hours, 4 μL of Lysis buffer reagent was added to the plate, which was then centrifuged for 1 minute at 1000 rpm. Then the plate was incubated at room temperature for 30 minutes. After 30 minutes, 4 μL of antibody mix containing Eu Cryptate, d2 Cryptate, and detection buffer, was added to the plate. The plate was centrifuged for 1 minute at 1000 rpm and then incubated overnight at room temperature. The plate was read on an Envision plate reader using the HTRF protocol. The percentage of inhibition of AKT phosphorylation was calculated using the following formula: % Inhibition=100×(pAKTHC−pAKTSample)/(pAKTHC−pAKTLC)), where pAKTHC is obtained from cells treated with 0.1% DMSO only; PAKTLC is obtained from cells treated with 10 uM Alpelisib. The IC50 (concentration achieving 50% inhibition of pAKT) is calculated by fitting the Curve using XIfit (v5.3.1.3), equation 201: Y=Bottom+(Top−Bottom)/(1+10\hat{ }((LogIC50−X)*HillSlope)).

### Reagent Table:

| Reagent | Vendor | Cat No. |
|---|---|---|
| Gibco RPMI 1640 Medium, no phenol red | Fisher | 11835030 |
| Gibco RPMI 1640 Medium | Fisher | 11875093 |
| McCoy's 5A (Modified) Medium, HEPES | Fisher | 12330031 |
| DMEM, high glucose, HEPES, no phenol red | Fisher | 21063045 |
| Gibco Trypsin-EDTA (0.5%), no phenol red | Fisher | 15400054 |
| PBS | Solarbio | P1020 |
| Trypan blue solution 0.4% | Fisher | T10282 |
| Fetal Bovine Serum Australia origin | Sigma | 82051-458 |
| ProxiPlate-384 Plus, White, TC treated, Case of 160 | Perkin Elmer | 6008238 |
| pAKT (Ser473) HTRF | Cisbio | 64AKSPEH |

For EC50 values shown in Table 2, “A” refers to 1 nM<EC50<500 nM; “B” refers to 500 nM<EC50<2 μM; “C” refers to 2 μM<EC50<15 μM; and “D” refers to an EC50>15 μM.

TABLE 2
Cellular proliferation data

| Example # | Avg T-47D EC50 | Avg SKBR3 EC50 |
|---|---|---|
| 1 | A | C |
| 2 | C | D |
| 3 | A | C |
| 4 | C | C |
| 5 | C | D |
| 6 | A | C |
| 7 | C | D |
| 8 | A | C |
| 9 | C | D |
| 10 | C | C |
| 11 | A | C |
| 12 | D | D |
| 13 | B | D |
| 14 | A | D |
| 15 | B | D |
| 16 | C | D |
| 17 | A | D |
| 18 | C | C |
| 19 | A | C |
| 20 | C | D |
| 21 | A | D |
| 22 | C | C |
| 23 | A | C |
| 24 | A | C |
| 25 | A | C |
| 26 | A | C |
| 27 | A | C |
| 28 | B | D |
| 29 | C | D |
| 30 | C | C |
| 31 | A | C |
| 32 | D | D |
| 33 | A | D |
| 34 | A | D |
| 35 | D | D |
| 36 | A | D |
| 37 | C | D |
| 38 | A | D |
| 39 | A | D |
| 40 | B | D |
| 41 | A | C |
| 42 | B | D |
| 43 | A | D |
| 44 | A | C |
| 45 | C | D |
| 46 | B | C |
| 47 | A | D |
| 48 | B | D |
| 49 | A | D |
| 50 | C | D |
| 51 | C | D |
| 52 | A | C |
| 53 | C | D |
| 54 | C | D |
| 55 | B | D |
| 56 | C | D |
| 57 | C | D |
| 58 | D | D |
| 59 | B | D |
| 60 | B | D |
| 61 | B | D |
| 62 | B | D |
| 63 | C | D |
| 64 | B | D |
| 65 | B | D |
| 66 | A | D |
| 67 | B | D |
| 68 | B | D |
| 69 | B | D |
| 70 | B | D |
| 71 | B | D |
| 72 | C | D |
| 73 | B | D |
| 74 | C | D |
| 75 | B | D |
| 76 | D | D |
| 77 | B | D |
| 78 | C | D |
| 79 | A | D |
| 80 | D | D |
| 81 | B | D |
| 82 | B | D |
| 83 | C | D |
| 84 | B | D |
| 85 | A | D |
| 86 | C | D |
| 87 | C | D |
| 88 | B | C |
| 89 | C | D |
| 90 | B | D |
| 91 | A | C |
| 92 | B | D |
| 93 | C | D |
| 94 | A | C |
| 95 | B | D |
| 96 | A | C |
| 97 | C | D |
| 98 | B | D |
| 99 | A | C |
| 100 | A | D |
| 101 | D | D |
| 102 | C | D |
| 103 | A | D |
| 104 | C | C |
| 105 | B | D |
| 106 | C | D |
| 107 | A | D |
| 108 | C | D |
| 109 | A | C |
| 110 | C | C |
| 111 | A | C |
| 112 | C | D |
| 113 | A | C |
| 114 | D | D |
| 115 | A | D |
| 116 | B | D |
| 117 | C | D |
| 118 | A | C |
| 119 | A | C |
| 120 | A | C |
| 121 | B | D |
| 122 | C | D |
| 123 | D | D |
| 124 | B | D |
| 125 | C | D |
| 126 | A | D |
| 127 | C | D |
| 128 | A | C |
| 129 | A | C |
| 130 | A | C |
| 131 | C | D |
| 132 | A | D |
| 133 | C | D |
| 134 | C | D |
| 135 | C | D |
| 136 | A | C |
| 137 | B | D |
| 138 | D | D |
| 139 | B | D |
| 140 | A | D |
| 141 | D | D |
| 142 | B | D |
| 143 | D | D |
| 144 | D | D |
| 145 | A | C |
| 146 | C | D |
| 147 | A | D |
| 148 | B | D |
| 149 | A | C |
| 150 | C | D |
| 151 | D | D |
| 152 | B | D |
| 153 | A | C |
| 154 | C | D |
| 155 | A | D |
| 156 | C | D |
| 157 | A | D |
| 158 | A | D |
| 159 | C | D |
| 160 | A | D |
| 161 | A | C |
| 162 | A | D |
| 163 | A | C |
| 164 | A | C |
| 165 | B | D |
| 166 | C | D |
| 167 | A | C |
| 168 | A | D |
| 169 | D | D |
| 170 | A | C |
| 171 | B | D |
| 172 | A | D |
| 173 | A | C |
| 174 | B | D |
| 175 | A | C |
| 176 | A | C |
| 177 | C | D |
| 178 | B | D |
| 179 | A | D |
| 180 | A | D |
| 181 | D | D |
| 182 | C | D |
| 183 | D | D |
| 184 | B | D |
| 185 | A | D |
| 186 | A | C |
| 187 | A | C |
| 188 | A | C |
| 189 | A | D |
| 190 | A | D |
| 191 | B | D |
| 192 | A | D |
| 193 | B | D |
| 194 | B | D |
| 195 | B | D |
| 196 | A | D |
| 197 | A | D |
| 198 | B | D |
| 199 | A | C |
| 200 | B | D |
| 201 | A | D |
| 202 | A | D |
| 203 | A | D |
| 204 | A | D |
| 205 | A | D |
| 206 | A | C |
| 207 | A | D |
| 208 | A | D |
| 209 | B | D |
| 210 | B | D |
| 211 | B | D |
| 212 | B | D |
| 213 | B | D |
| 214 | A | D |
| 215 | A | D |
| 216 | A | D |
| 217 | A | C |
| 218 | A | C |
| 219 | A | C |
| 220 | A | D |
| 221 | A | D |
| 222 | C | D |
| 223 | B | D |
| 224 | A | D |
| 225 | A | D |
| 226 | A | D |
| 227 | A | D |
| 228 | A | D |
| 229 | A | D |
| 230 | A | D |
| 231 | A | C |
| 232 | A | C |
| 233 | A | C |
| 234 | A | D |
| 235 | A | C |
| 236 | A | C |
| 237 | A | C |
| 238 | A | D |
| 239 | A | D |
| 240 | A | D |
| 241 | A | D |
| 242 | A | D |
| 243 | A | D |
| 244 | A | D |
| 245 | A | D |
| 246 | C | D |
| 247 | A | D |
| 248 | B | D |
| 249 | A | D |
| 250 | A | D |
| 251 | A | D |
| 252 | A | D |
| 253 | B | D |
| 254 | C | D |
| 255 | A | C |
| 256 | A | C |
| 257 | A | C |
| 258 | A | D |
| 259 | A | D |
| 260 | B | D |
| 261 | A | D |
| 262 | A | D |
| 263 | A | D |
| 264 | A | D |
| 265 | A | D |
| 266 | A | D |
| 267 | A | C |
| 268 | A | D |
| 269 | A | C |
| 270 | B | D |
| 271 | A | C |
| 272 | A | C |
| 273 | B | D |
| 274 | C | D |
| 275 | A | D |
| 276 | A | D |
| 277 | A | C |
| 278 | A | D |
| 279 | A | D |
| 280 | A | D |
| 281 | A | D |
| 282 | A | C |
| 283 | A | D |
| 284 | A | D |
| 285 | A | C |
| 286 | A | D |
| 287 | A | C |
| 288 | A | D |
| 289 | A | D |
| 290 | A | D |
| 291 | A | D |
| 292 | A | D |
| 293 | A | C |
| 294 | A | D |
| 295 | A | D |
| 296 | A | D |
| 297 | C | D |
| 298 | A | D |
| 299 | A | C |
| 300 | B | D |
| 301 | A | D |
| 302 | A | D |
| 303 | A | D |
| 304 | A | D |
| 305 | A | D |
| 306 | A | D |
| 307 | A | D |
| 308 | A | C |
| 309 | A | D |
| 310 | A | C |
| 311 | A | D |
| 312 | A | C |
| 313 | A | D |
| 314 | A | D |
| 315 | A | D |
| 316 | A | D |
| 317 | A | D |
| 318 | A | C |
| 319 | A | D |
| 320 | A | D |
| 321 | A | D |
| 322 | A | D |
| 323 | A | C |
| 324 | A | C |
| 325 | A | C |
| 326 | A | D |
| 327 | B | D |
| 328 | B | C |
| 329 | A | C |
| 330 | B | D |
| 331 | A | D |
| 332 | B | D |
| 333 | A | D |
| 334 | A | D |
| 335 | B | D |
| 336 | A | C |
| 337 | A | D |
| 338 | A | C |
| 339 | B | D |
| 340 | A | D |
| 341 | A | D |
| 342 | A | D |
| 343 | A | C |
| 344 | A | D |
| 345 | A | D |
| 346 | A | D |
| 347 | A | D |
| 348 | A | D |
| 349 | A | D |
| 350 | A | D |
| 351 | A | D |
| 352 | A | C |
| 353 | A | C |
| 354 | C | D |
| 355 | A | D |
| 356 | A | D |
| 357 | A | C |
| 358 | A | C |
| 359 | A | D |
| 360 | C | D |
| 361 | A | D |
| 362 | A | C |
| 363 | A | D |
| 364 | A | C |
| 365 | A | D |
| 366 | A | C |
| 367 | A | C |
| 368 | A | C |
| 369 | A | D |
| 370 | A | D |
| 371 | B | D |
| 372 | C | D |
| 373 | A | D |
| 374 | A | D |
| 375 | A | D |
| 376 | A | D |
| 377 | C | D |
| 378 | A | D |
| 379 | A | D |
| 380 | A | D |
| 381 | A | C |
| 382 | A | D |
| 383 | A | D |
| 384 | A | D |
| 385 | A | D |
| 386 | C | D |
| 387 | A | D |
| 388 | A | D |
| 389 | A | D |
| 390 | A | D |
| 391 | C | D |
| 392 | B | D |
| 393 | A | D |
| 394 | A | D |
| 395 | A | D |
| 396 | A | D |
| 397 | A | D |
| 398 | A | D |
| 399 | B | D |
| 400 | B | D |
| 401 | A | C |
| 402 | C | D |
| 403 | A | D |
| 404 | A | D |
| 405 | B | D |
| 406 | A | D |
| 407 | A | D |
| 408 | C | D |
| 409 | B | D |
| 410 | A | D |
| 411 | A | D |
| 412 | C | D |
| 413 | C | D |
| 414 | A | D |
| 415 | B | D |
| 416 | B | D |
| 417 | A | D |
| 418 | D | D |
| 419 | B | D |
| 420 | C | D |
| 421 | B | D |
| 422 | A | D |
| 423 | A | D |
| 424 | A | D |
| 425 | B | D |
| 426 | A | D |
| 427 | B | D |
| 428 | B | D |
| 429 | A | D |
| 430 | A | D |
| 431 | A | D |
| 432 | B | D |
| 433 | B | D |
| 434 | A | D |
| 435 | C | D |
| 436 | C | D |
| 437 | B | D |
| 438 | D | D |
| 439 | B | D |
| 440 | A | D |
| 441 | B | D |
| 442 | A | D |
| 443 | A | D |
| 444 | B | C |
| 445 | C | D |
| 446 | A | D |
| 447 | A | D |
| 448 | A | D |
| 449 | A | D |
| 450 | A | D |
| 451 | A | D |
| 452 | A | D |
| 453 | A | C |
| 454 | A | D |
| 455 | A | D |
| 456 | A | C |
| 457 | A | D |
| 458 | A | D |
| 459 | A | C |
| 460 | A | B |
| 461 | A | D |
| 462 | A | D |
| 463 | A | C |
| 464 | A | C |
| 465 | A | C |
| 466 | A | D |
| 467 | A | C |
| 468 | A | D |
| 469 | A | C |
| 470 | A | D |
| 471 | A | C |
| 472 | A | C |
| 473 | A | D |
| 474 | A | D |
| 475 | B | D |
| 476 | B | D |
| 477 | A | D |
| 478 | A | D |
| 479 | C | D |
| 480 | B | D |
| 481 | B | D |
| 482 | A | D |
| 483 | B | D |
| 484 | B | D |
| 485 | C | C |
| 486 | C | D |
| 487 | A | C |
| 488 | A | D |
| 489 | A | D |
| 490 | A | C |
| 491 | A | C |
| 492 | A | C |
| 493 | A | D |
| 494 | A | D |
| 495 | A | C |
| 496 | B | D |
| 497 | A | D |
| 498 | B | D |
| 499 | B | D |
| 500 | A | C |
| 501 | A | D |
| 502 | A | C |
| 503 | B | D |
| 504 | A | D |
| 505 | A | D |
| 506 | A | D |
| 507 | B | D |
| 508 | B | D |
| 509 | A | C |
| 510 | A | D |
| 511 | A | C |
| 512 | A | C |
| 513 | A | D |
| 514 | A | D |
| 515 | A | C |
| 516 | A | D |
| 517 | A | D |
| 518 | A | D |
| 519 | A | D |
| 520 | A | D |
| 521 | A | C |
| 522 | A | D |
| 523 | B | D |
| 524 | A | D |
| 525 | A | D |
| 526 | A | D |
| 527 | A | D |
| 528 | A | C |
| 529 | A | D |
| 530 | A | D |
| 531 | A | D |
| 532 | A | D |
| 533 | A | D |
| 534 | A | D |
| 535 | A | D |
| 536 | A | D |
| 537 | A | D |
| 538 | A | D |
| 539 | A | D |
| 540 | A | D |
| 541 | A | C |
| 542 | A | C |
| 543 | A | D |
| 544 | A | D |
| 545 | A | D |
| 546 | A | D |
| 547 | B | D |
| 548 | A | C |
| 549 | A | B |
| 550 | A | C |
| 551 | A | C |
| 552 | A | C |
| 553 | A | C |
| 554 | A | D |
| 555 | C | D |
| 556 | A | C |
| 557 | B | C |
| 558 | A | C |
| 559 | B | D |
| 560 | A | C |
| 561 | A | D |
| 562 | A | D |
| 563 | A | D |
| 564 | A | C |
| 565 | A | D |
| 566 | C | C |
| 567 | A | C |
| 568 | A | D |
| 569 | B | C |
| 570 | A | D |
| 571 | A | C |
| 572 | A | D |
| 573 | C | D |
| 574 | A | C |
| 575 | C | D |
| 576 | A | C |
| 577 | A | D |
| 578 | A | C |
| 579 | A | C |
| 580 | A | C |
| 581 | A | D |
| 582 | A | D |

For IC50 values shown in Table 3 “A” refers to 1 nM<IC50<500 nM; “B” refers to 500 nM<IC50<2 μM; “C” refers to 2 μM<IC50<15 μM; and “D” refers to an IC50>15 μM.

TABLE 3
Cellular pAKT data

| Example # | Avg T-47D IC50 | Avg SKBR3 IC50 |
|---|---|---|
| 1 | A | D |
| 2 | D | D |
| 3 | B | C |
| 4 | C | D |
| 5 | C | D |
| 6 | A | C |
| 7 | B | D |
| 8 | A | C |
| 9 | C | D |
| 10 | D | D |
| 11 | A | C |
| 12 | D | D |
| 13 | B | D |
| 14 | A | D |
| 15 | C | D |
| 16 | D | D |
| 17 | A | D |
| 18 | D | D |
| 19 | A | D |
| 20 | D | D |
| 21 | A | D |
| 22 | C | D |
| 23 | B | D |
| 24 | A | C |
| 25 | A | C |
| 26 | A | C |
| 27 | A | C |
| 28 | A | D |
| 29 | D | D |
| 30 | D | D |
| 31 | A | D |
| 32 | D | D |
| 33 | A | D |
| 34 | A | D |
| 35 | D | D |
| 36 | A | D |
| 37 | C | D |
| 38 | A | D |
| 39 | A | D |
| 40 | A | D |
| 41 | A | D |
| 42 | B | D |
| 43 | A | D |
| 44 | A | C |
| 45 | B | D |
| 46 | A | D |
| 47 | A | D |
| 48 | A | D |
| 49 | A | D |
| 50 | | |
| 51 | | |
| 52 | | |
| 53 | | |
| 54 | | |
| 55 | A | D |
| 56 | B | D |
| 57 | A | D |
| 58 | D | D |
| 59 | B | D |
| 60 | A | D |
| 61 | A | D |
| 62 | B | D |
| 63 | A | D |
| 64 | A | D |
| 65 | A | D |
| 66 | A | D |
| 67 | A | D |
| 68 | A | D |
| 69 | A | D |
| 70 | B | D |
| 71 | A | C |
| 72 | B | D |
| 73 | A | D |
| 74 | B | D |
| 75 | A | D |
| 76 | D | D |
| 77 | A | D |
| 78 | D | D |
| 79 | A | D |
| 80 | D | D |
| 81 | A | D |
| 82 | A | D |
| 83 | D | D |
| 84 | A | D |
| 85 | A | C |
| 86 | B | D |
| 87 | C | D |
| 88 | B | D |
| 89 | B | D |
| 90 | B | D |
| 91 | A | D |
| 92 | A | D |
| 93 | D | D |
| 94 | A | C |
| 95 | B | D |
| 96 | A | D |
| 97 | C | D |
| 98 | A | D |
| 99 | A | D |
| 100 | A | D |
| 101 | D | D |
| 102 | D | D |
| 103 | A | D |
| 104 | D | D |
| 105 | A | D |
| 106 | D | D |
| 107 | A | D |
| 108 | D | D |
| 109 | A | D |
| 110 | D | D |
| 111 | A | C |
| 112 | D | D |
| 113 | B | D |
| 114 | D | D |
| 115 | D | D |
| 116 | B | D |
| 117 | D | D |
| 118 | A | C |
| 119 | A | D |
| 120 | A | D |
| 121 | A | D |
| 122 | D | D |
| 123 | D | D |
| 124 | C | D |
| 125 | D | D |
| 126 | A | D |
| 127 | D | D |
| 128 | A | C |
| 129 | A | C |
| 130 | A | C |
| 131 | D | D |
| 132 | A | D |
| 133 | D | D |
| 134 | D | D |
| 135 | D | D |
| 136 | A | C |
| 137 | A | D |
| 138 | D | D |
| 139 | B | D |
| 140 | A | D |
| 141 | D | D |
| 142 | C | D |
| 143 | D | D |
| 144 | B | D |
| 145 | A | C |
| 146 | D | D |
| 147 | A | C |
| 148 | B | D |
| 149 | A | C |
| 150 | C | D |
| 151 | D | D |
| 152 | A | D |
| 153 | A | C |
| 154 | D | D |
| 155 | A | D |
| 156 | C | D |
| 157 | A | D |
| 158 | A | D |
| 159 | B | D |
| 160 | A | D |
| 161 | A | C |
| 162 | A | C |
| 163 | A | C |
| 164 | A | C |
| 165 | A | D |
| 166 | C | D |
| 167 | A | C |
| 168 | A | C |
| 169 | C | D |
| 170 | A | C |
| 171 | A | D |
| 172 | A | D |
| 173 | A | D |
| 174 | C | D |
| 175 | A | D |
| 176 | A | D |
| 177 | D | D |
| 178 | A | D |
| 179 | A | D |
| 180 | A | D |
| 181 | D | D |
| 182 | C | D |
| 183 | D | D |
| 184 | A | D |
| 185 | A | C |
| 186 | A | C |
| 187 | A | D |
| 188 | A | B |
| 189 | A | D |
| 190 | A | D |
| 191 | A | D |
| 192 | A | D |
| 193 | A | D |
| 194 | A | D |
| 195 | A | D |
| 196 | A | C |
| 197 | A | C |
| 198 | B | D |
| 199 | A | C |
| 200 | B | D |
| 201 | A | D |
| 202 | A | D |
| 203 | A | D |
| 204 | A | D |
| 205 | A | D |
| 206 | A | B |
| 207 | A | D |
| 208 | A | D |
| 209 | A | D |
| 210 | A | D |
| 211 | A | D |
| 212 | B | D |
| 213 | A | D |
| 214 | A | D |
| 215 | A | D |
| 216 | A | D |
| 217 | A | D |
| 218 | A | C |
| 219 | A | C |
| 220 | A | C |
| 221 | A | C |
| 222 | C | D |
| 223 | A | D |
| 224 | A | D |
| 225 | A | D |
| 226 | A | D |
| 227 | A | D |
| 228 | A | D |
| 229 | A | D |
| 230 | A | D |
| 231 | A | C |
| 232 | A | C |
| 233 | A | C |
| 234 | A | D |
| 235 | B | C |
| 236 | A | C |
| 237 | A | C |
| 238 | A | D |
| 239 | A | D |
| 240 | A | D |
| 241 | A | D |
| 242 | A | D |
| 243 | A | D |
| 244 | A | C |
| 245 | A | D |
| 246 | C | D |
| 247 | A | D |
| 248 | A | D |
| 249 | A | D |
| 250 | A | D |
| 251 | A | D |
| 252 | A | D |
| 253 | B | D |
| 254 | C | D |
| 255 | A | C |
| 256 | A | C |
| 257 | A | C |
| 258 | A | C |
| 259 | A | C |
| 260 | C | D |
| 261 | A | D |
| 262 | A | D |
| 263 | A | D |
| 264 | A | D |
| 265 | A | D |
| 266 | A | D |
| 267 | A | D |
| 268 | A | D |
| 269 | A | C |
| 270 | A | D |
| 271 | A | D |
| 272 | A | C |
| 273 | A | D |
| 274 | C | D |
| 275 | A | D |
| 276 | A | D |
| 277 | A | C |
| 278 | A | D |
| 279 | A | D |
| 280 | A | D |
| 281 | A | D |
| 282 | A | D |
| 283 | A | D |
| 284 | A | C |
| 285 | A | C |
| 286 | A | D |
| 287 | A | C |
| 288 | A | D |
| 289 | A | D |
| 290 | A | D |
| 291 | A | D |
| 292 | A | D |
| 293 | A | C |
| 294 | A | D |
| 295 | A | D |
| 296 | A | D |
| 297 | C | D |
| 298 | A | C |
| 299 | A | C |
| 300 | B | D |
| 301 | A | D |
| 302 | A | D |
| 303 | A | D |
| 304 | A | D |
| 305 | A | D |
| 306 | A | D |
| 307 | A | D |
| 308 | A | C |
| 309 | A | C |
| 310 | A | D |
| 311 | A | D |
| 312 | A | D |
| 313 | A | D |
| 314 | A | D |
| 315 | A | D |
| 316 | A | D |
| 317 | A | D |
| 318 | A | C |
| 319 | A | D |
| 320 | A | D |
| 321 | A | C |
| 322 | A | D |
| 323 | A | C |
| 324 | A | D |
| 325 | B | C |
| 326 | A | D |
| 327 | B | C |
| 328 | B | D |
| 329 | A | C |
| 330 | A | D |
| 331 | A | D |
| 332 | A | D |
| 333 | A | D |
| 334 | A | D |
| 335 | A | D |
| 336 | A | C |
| 337 | A | D |
| 338 | A | D |
| 339 | A | D |
| 340 | A | C |
| 341 | A | D |
| 342 | A | D |
| 343 | A | C |
| 344 | A | D |
| 345 | A | D |
| 346 | A | D |
| 347 | A | D |
| 348 | A | D |
| 349 | A | D |
| 350 | A | D |
| 351 | A | D |
| 352 | A | C |
| 353 | A | B |
| 354 | C | D |
| 355 | A | D |
| 356 | A | D |
| 357 | A | C |
| 358 | A | D |
| 359 | A | D |
| 360 | D | D |
| 361 | A | D |
| 362 | A | C |
| 363 | A | D |
| 364 | A | B |
| 365 | A | D |
| 366 | A | C |
| 367 | A | C |
| 368 | A | C |
| 369 | A | D |
| 370 | A | D |
| 371 | B | D |
| 372 | C | D |
| 373 | A | D |
| 374 | A | D |
| 375 | A | D |
| 376 | A | C |
| 377 | C | D |
| 378 | A | D |
| 379 | A | D |
| 380 | A | D |
| 381 | A | C |
| 382 | A | D |
| 383 | A | D |
| 384 | A | D |
| 385 | A | D |
| 386 | C | D |
| 387 | B | D |
| 388 | A | D |
| 389 | A | D |
| 390 | A | D |
| 391 | D | D |
| 392 | B | D |
| 393 | A | D |
| 394 | A | D |
| 395 | A | D |
| 396 | A | D |
| 397 | B | D |
| 398 | A | C |
| 399 | B | D |
| 400 | B | D |
| 401 | A | B |
| 402 | B | D |
| 403 | A | D |
| 404 | A | D |
| 405 | A | D |
| 406 | A | D |
| 407 | A | D |
| 408 | C | D |
| 409 | B | D |
| 410 | A | D |
| 411 | A | D |
| 412 | B | D |
| 413 | C | D |
| 414 | A | D |
| 415 | A | D |
| 416 | B | D |
| 417 | A | D |
| 418 | D | D |
| 419 | B | D |
| 420 | C | D |
| 421 | A | D |
| 422 | A | D |
| 423 | A | D |
| 424 | A | C |
| 425 | A | D |
| 426 | A | D |
| 427 | A | D |
| 428 | A | D |
| 429 | A | D |
| 430 | A | D |
| 431 | A | D |
| 432 | A | D |
| 433 | A | D |
| 434 | A | D |
| 435 | C | D |
| 436 | C | D |
| 437 | B | C |
| 438 | D | D |
| 439 | B | D |
| 440 | A | C |
| 441 | A | D |
| 442 | A | D |
| 443 | A | D |
| 444 | B | C |
| 445 | C | D |
| 446 | A | D |
| 447 | A | D |
| 448 | A | D |
| 449 | A | D |
| 450 | A | D |
| 451 | A | D |
| 452 | A | D |
| 453 | A | D |
| 454 | A | D |
| 455 | A | D |
| 456 | A | C |
| 457 | A | D |
| 458 | A | D |
| 459 | A | C |
| 460 | A | B |
| 461 | A | D |
| 462 | A | D |
| 463 | A | D |
| 464 | A | C |
| 465 | A | C |
| 466 | A | C |
| 467 | A | C |
| 468 | A | C |
| 469 | A | C |
| 470 | A | D |
| 471 | A | C |
| 472 | A | C |
| 473 | A | C |
| 474 | A | D |
| 475 | A | D |
| 476 | A | D |
| 477 | A | C |
| 478 | A | C |
| 479 | C | D |
| 480 | A | D |
| 481 | B | D |
| 482 | A | D |
| 483 | A | D |
| 484 | B | D |
| 485 | B | D |
| 486 | C | D |
| 487 | A | C |
| 488 | A | D |
| 489 | A | D |
| 490 | A | C |
| 491 | A | C |
| 492 | A | C |
| 493 | A | D |
| 494 | A | D |
| 495 | A | B |
| 496 | A | D |
| 497 | A | D |
| 498 | B | D |
| 499 | B | D |
| 500 | A | C |
| 501 | A | D |
| 502 | A | B |
| 503 | B | D |
| 504 | A | D |
| 505 | A | D |
| 506 | A | D |
| 507 | A | D |
| 508 | B | D |
| 509 | A | C |
| 510 | A | D |
| 511 | A | D |
| 512 | A | C |
| 513 | A | D |
| 514 | A | D |
| 515 | A | D |
| 516 | A | D |
| 517 | A | D |
| 518 | A | D |
| 519 | A | D |
| 520 | A | D |
| 521 | A | D |
| 522 | A | C |
| 523 | B | D |
| 524 | A | D |
| 525 | A | D |
| 526 | A | D |
| 527 | A | C |
| 528 | A | C |
| 529 | A | D |
| 530 | A | D |
| 531 | A | D |
| 532 | A | D |
| 533 | A | D |
| 534 | A | D |
| 535 | A | D |
| 536 | A | D |
| 537 | A | D |
| 538 | A | D |
| 539 | A | D |
| 540 | A | D |
| 541 | A | B |
| 542 | A | C |
| 543 | A | D |
| 544 | A | D |
| 545 | A | D |
| 546 | A | D |
| 547 | A | D |
| 548 | A | D |
| 549 | A | C |
| 550 | A | D |
| 551 | A | C |
| 552 | A | C |
| 553 | A | C |
| 554 | A | D |
| 555 | B | D |
| 556 | A | C |
| 557 | A | D |
| 558 | A | D |
| 559 | B | D |
| 560 | A | D |
| 561 | A | D |
| 562 | A | C |
| 563 | A | D |
| 564 | A | D |
| 565 | A | D |
| 566 | D | D |
| 567 | A | C |
| 568 | A | D |
| 569 | A | D |
| 570 | A | D |
| 571 | A | D |
| 572 | A | C |
| 573 | C | D |
| 574 | A | D |
| 575 | B | D |
| 576 | A | D |
| 577 | A | D |
| 578 | A | C |
| 579 | A | D |
| 580 | A | C |
| 581 | A | D |
| 582 | A | D |

CD1 mice were dosed with a single IV or PO dose, followed by serial sampling of plasma at 0.0833 (IV only), 0.25, 0.5, 1, 2, 4, 8, 24 hours post dose. Desired serial concentrations of working solutions were achieved by diluting stock solution of analyte with 50% acetonitrile in water solution.10 μL of working solutions (0.5, 1, 2, 5, 10, 50, 100, 500, 1000 ng/ml) were added to 10 μL of the blank Female CD1 Mouse plasma to achieve calibration standards of 0.5˜1000 ng/ml (0.5, 1, 2, 5, 10, 50, 100, 500, 1000 ng/ml) in a total volume of 20 μL. Five quality control samples at 1 ng/ml, 2 ng/ml, 5 ng/ml, 50 ng/ml and 800 ng/ml for plasma were prepared independently of those used for the calibration curves. These QC samples were prepared on the day of analysis in the same way as calibration standards. 20 μL standards, 20 μL QC samples and 20 μL unknown samples (10 μL plasma with 10 μL blank solution) were added to 200 μL of acetonitrile containing IS mixture for precipitating protein respectively. Then the samples were vortexed for 30 s. after centrifugation at 4° Celsius, 4000 rpm for 15 min. The supernatant was diluted with water at a ratio of 1:2 (V/V, 1:2), Then 5 μL of diluted supernatant was injected into the LC/MS/MS system for quantitative analysis. The results are shown in Table 4.

TABLE 4
mouse pharmacokinetic data

| Example # | 1 | 8 | 23 |
|---|---|---|---|
| PK-Mouse: | 1 | 1 | 1 |
| IV Dose | | | |
| (mg/kg) | | | |
| PK-Mouse: | 20% PEG400/20% PG/ | 20% PEG400/20% PG/ | 20% PEG400/20% PG/ |
| IV Vehicle | 60% of 20% HPβCD | 60% of 20% HPβCD | 60% of 20% HPβCD |
| | solution in water | solution in water | solution in water |
| PK-Mouse: | 5.7 | 11.7 | 24.6 |
| IV Cl | | | |
| (mL/min/kg) | | | |
| PK-Mouse: | 3064 | 1438 | 683 |
| IV AUC last | | | |
| (h*ng/mL) | | | |
| PK-Mouse | 10 | 10 | 10 |
| PO Dose | | | |
| (mg/kg) | | | |
| PK-Mouse: | 30% SBEβCD in water | 30% SBEβCD in water | 30% SBEβCD in water |
| PO Vehicle | | | |
| PK-Mouse | 15215 | 8896 | 7230 |
| PO AUC last | | | |
| (h*ng/mL) | | | |
| PK-Mouse: | 50 | 62 | 106 |
| PO F (%) | | | |

### REFERENCES

Ali, K., Bilancio, A., Thomas, M., Pearce, W., Gilfillan, A. M., Tkaczyk, C., Kuehn, N., Gray, A., Giddings, J., Peskett, E., Fox, R., Bruce, I., Walker, C., Sawyer, C., Okkenhaug, K., Finan, P., and Vanhaesebroeck, B. 2004. Essential role for the p110delta phosphoinositide 3-kinase in the allergic response. Nature 431(7011): 1007-1011.
Backer, J. M. 2016. The intricate regulation and complex functions of the Class III phosphoinositide 3-kinase Vps34. Biochem J. 473(15): 2251-2271.
Castel, P., Toska, E., Engelman, J. A., and Scaltriti, M. 2021. The present and future of PI3K inhibitors for cancer therapy. Nat Cancer 2(6): 587-597.
Fruman, D. A., Chiu H., Hopkins B. D., Bagrodia, S., Cantley, L. C., and Abraham R. T. 2017. The PI3K pathway in human disease. Cell 170(4): 605-635.
Gadkar, K., Friedrich, C., Hurez, V., Ruiz, M. L., Dickmann, L., Jolly, M. K., Schutt, L., Jin, J., Ware, J. A., and Ramanujan, S. 2021. Quantitative systems pharmacology model-based investigation of adverse gastrointestinal events associated with prolonged treatment with PI3-kinase inhibitors. CPT Pharmacometrics Syst Pharmacol. 11(5): 616-627.
Goncalves, M. D., Hopkins, B. D., and Cantley, L. C. 2018. Phosphatidylinositol 3-Kinase, growth disorders, and cancer. N Engl J Med. 379(21): 2052-2062.
Hang, H. C. and Bertozzi, C. R. 2001. Chemoselective approaches to glycoproteon assembly. Accounts of Chemical Research 34(9): 727-736.
Hawkins, P. T. and Stephens, L. R. 2015. PI3K signalling in inflammation. 2015. Biochimica et Biophysica Acta 1851(6): 882-897.
Hopkins, B. D., Pauli, C., Du, X., Wang, D. G., Li, X., Wu, D., Amadiume, S. C., Goncalves, M. D., Hodakoski, C., Lundquist, M. R., Bareja, R., Ma, Y., Harris, E. M., Sboner, A., Beltran, H., Rubin, M. A., Mukherjee, S., and Cantley, L. C. 2018. Suppression of insulin feedback enhances the efficacy of PI3K inhibitors. Nature 560(7719): 499-503.
Jackson, S. P., Schoenwaelder, S. M., Goncalves, I., Nesbitt, W. S., Yap, C. L., Wright, C. E., Kenche, V., Anderson, K. E., Dopheide, S. M., Yuan, Y., Sturgeon, S. A., Prabaharan, H., Thompson, P. E., Smith, G. D., Shepherd, P. R., Daniele, N., Kulkarni, S., Abbott, B., Saylik, D., Jones, C., Lu, L., Giuliano, S., Hughan, S. C., Angus, J. A., Robertson, A. D., and Salem, H. H. 2005. PI 3-kinase p110beta: a new target for antithrombotic therapy. Nat Med. 11(5): 507-514.
Jiang, N., Dai, Q., Su, X., Fu, J., and Feng, X. 2020. Role of PI3K/AKT pathway in cancer: the framework of malignant behavior. Mol Biol Rep. 47(6): 4587-4629.
Keppler-Noreuil, K. M., Rios, J. J., Parker, V. E., Semple, R. K., Lindhurst, M. J.; Sapp, J. C., Alomari, A., Ezaki, M., Dobyns, W., and Biesecker, L. G. 2015. PIK3CA-related overgrowth spectrum (PROS): diagnostic and testing eligibility criteria, differential diagnosis, and evaluation. Am J Med Genet A. 167A(2): 287-295.
Kiick, K. L., Saxon, E., Tirrell, D. A., and Bertozzi, C. R. 2002. Incorporation of azides into recombinant proteins for chemoselective modification by the Staudinger ligation. Proc Natl Acad Sci USA. 99(1): 19-24.
Klaus, S., Neumann, H., Zapf, A., Strübing, D., Hübner, S., Almena, J., Riermeier, T., Groß, P., Sarich, M., Krahnert, W., Rossen, K., and Beller, M. 2006. A general and efficient method for the formylation of aryl and heteroaryl bromides. Ang Chem Int Ed. 45(1): 154-158.
Kurek, K. C., Luks, V. L., Ayturk, U. M., Alomari, A. I., Fishman, S. J., Spencer S. A., Mulliken, J. B., Bowen, M. E., Yamamoto, G. L., Kozakewich, H. P., and Warman, M. L. 2012. Somatic mosaic activating mutations in PIK3CA cause CLOVES syndrome. Am J Hum Genet. 90(6): 1108-1115.
Lemieux, G. A. and Bertozzi, C. R. 1998. Chemoselective ligation reactions with proteins, oligosaccharides and cells. Trends in Biotechnology 16(12): 506-513.
Liu, P., Cheng, H., Roberts, T. M., and Zhao, J. J. 2009. Targeting the phosphoinositide 3-kinase pathway in cancer. Nat Rev Drug Discov. 8(8): 627-644.
Okkenhaug, K., Bilancio, A., Farjot, G., Priddle, H., Sancho, S., Peskett, E., Pearce, W., Meek, S. E., Salpekar, A., Waterfield, M. D., Smith, A. J., and Vanhaesebroeck, B. 2002. Impaired B and T cell antigen receptor signaling in p110delta PI 3-kinase mutant mice. Science 297(5583): 1031-1034.
Porta, C., Paglino, C., and Mosca, A. 2014. Targeting PI3K/Akt/mTOR signaling in cancer. Front Oncol. 4(64): 1-11.
Posor, Y., Eichhorn-Grünig, M., and Haucke, V. 2015. Phosphoinositides in endocytosis. Biochim Biophys Acta 1851(6): 794-804.
Prakash, G., Krishnamurti, R., and Olah, G. 1989. Synthetic methods and reactions. 141. Fluoride-induced trifluoromethylation of carbonyl compounds with trifluormethyltrimethylsilane (TMS-CF₃). A trifluoromethide equivalent. J Am Chem Soc. 111(1): 393-395.
Reichel, M. and Karaghiosoff, K. 2020. Reagents for selective fluoromethylation: A challenge in organofluorine chemistry. Ang Chem Int Ed. 59(30): 12268-12281.
Rugo, H. S., Lacouture, M. E., Goncalves, M. D., Masharani, U., Aapro, M. S., and O'Shaughnessy, J. A. 2022. A multidisciplinary approach to optimizing care of patients treated with alpelisib. The Breast 61:156-167.
Soler, A., Serra, H., Pearce, W., Angulo, A., Guillermet-Guibert, J., Friedman, L. S., Viñals, F., Gerhardt, H., Casanovas, O., Graupera, M., and Vanhaesebroeck, B. 2004. Inhibition of the p110α isoform of PI 3-kinase stimulates nonfunctional tumor angiogenesis. J Exp Med. 210(10): 1937-1945.
Thorpe, L. M., Yuzugullu, H., and Zhao, J. J. 2015. PI3K in cancer: divergent roles of isoforms, modes of activation and therapeutic targeting. Nat Rev Cancer 15(1): 7-24.
Vanhaesebroeck, B., Whitehead M. A., and Piñeiro, R. 2016. Molecules in medicine mini-review: isoforms of PI3K in biology and disease. J Mol Med (Berl). 94(1): 5-11.
Yang, J., Nie, J., Ma, X., Wei, Y., Peng, Y., and Wei, X. 2019. Targeting PI3K in cancer: mechanisms and advances in clinical trials. Mol Cancer 18(26): 1-28.
Zhao, Y., Huang, W., Zheng, J., and Hu, J. 2011. Efficient and direct nucleophilic difluoromethylation of carbonyl compounds and imines with Me₃SiCF₂H at ambient or low temperature. Org. Lett. 13(19): 5342-5345.

1.-39. (canceled)

40. A compound of Formula (1) 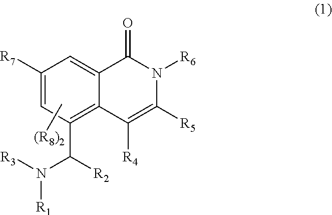
or a solvate, enantiomer, diastereomer, tautomer, polymorph or isotope-labeled compound thereof, or a pharmaceutically acceptable salt thereof,
wherein:
R₁ is alkyl, cycloalkyl, heterocyclyl, aryl or heteroaryl, where each of the alkyl, cycloalkyl, heterocyclyl, aryl or heteroaryl is unsubstituted or substituted;
R₂ is H, C₁-C₄ alkyl, C₃-C₇ cycloalkyl, CF₃, CH₂F or CF₂H, and where R₂ is not H, the carbon atom attached to R₂ is a chiral center and exists as a (R)- and (S)-racemic mixture or as either the (R)- or (S)-enantiomer;
R₃ is H or C₁-C₄ alkyl;
R₄ is H, F, Cl or CH₃;
R₆ is H, C₁-C₄ alkyl, C₃-C₇ cycloalkyl, heteroaryl, CF₃, CH₂F or CF₂H;
R₇ is H, C₁-C₄ alkyl, C₃-C₇ cycloalkyl, halogen, CN, CF₃, OCF₃, CH₂F or CF₂H;
each R₅ is independently H, C₁-C₄ alkyl, C₃-C₇ cycloalkyl, halogen, CN, CF₃, OCF₃, CH₂F or CF₂H;
R₅ is
H;
halogen;
—O-L₁-L₂-L₃-L₄-L₅-L₆-L₇-R₉;
—S-L₁-L₂-L₃-L₄-L₅-L₆-L₇-R₉;
—S(O)-L₁-L₂-L₃-L₅-L₆-L₇-R₉;
—S(O)₂-L₁-L₂-L₃-L₅-L₆-L₇-R₉;
—(NR₁₀)-L₁-L₂-L₃-L₄-L₅-L₆-L₇-R₉; or
-L₈-L₉-L₁₀-L₁₁-L₁₂-R₁₄,
wherein:
each of L₁, L₂, L₃, L₆ and L₇ is independently (CHR₁₁), (CHR₁₁—O), (CHR₁₁—S), (C₃-C₇ cycloalkyl), (CH₂)₁₋₄ or a bond;
L₄ is C═O, C═S or a bond;
L₅ is NR₁₀, S, O or a bond;
R₉ is H, C(═O)R₁₂, C(═O)NR₁₂R₁₃, NR₁₂R₁₃, C(═O)OR₁₂, C₁-C₆ alkyl, C₁-C₆ fluoroalkyl, cycloalkyl, heterocyclyl, aryl or heteroaryl, where each of the C₁-C₆ alkyl, C₁-C₆ fluoroalkyl, cycloalkyl, heterocyclyl, aryl or heteroaryl is unsubstituted or substituted; or alternatively when NR₁₀ is present, R₉ and R₁₀ together with the attached nitrogen atom may form a substituted or unsubstituted ring;
each of R₁₀ and R₁₁ is independently H or C₁-C₄ alkyl (such as CH₃, CH₂CH₃ or CH(CH₃)₂), where the C₁-C₄ alkyl is unsubstituted or substituted;
each of R₁₂ and R₁₃ is independently H, C₁-C₆ alkyl, cycloalkyl, heterocyclyl, aryl or heteroaryl, where each of the C₁-C₆ alkyl, cycloalkyl, heterocyclyl, aryl or heteroaryl is unsubstituted or substituted; or alternatively, R₁₂ and R₁₃ together with the attached nitrogen atom may form a substituted or unsubstituted ring;
L₈ is (CHR₁₅), (CHR₁₅—O), (CHR₁₅—S), (CHR₁₅—NR₁₆), C═O, C═S or a bond;
L₉ is C₃-C₇ cycloalkyl that is optionally part of a bridged, fused or spiro ring system, C(R₁₅)═C(R₁₅), C═C or a bond;
L₁₀ is independently (CHR₁₅), O, S, (NCR₁₅), N(C═O) or a bond;
L₁₁ is (CHR₁₅), C═O, C═S or a bond;
L₁₂ is H, (C₃-C₇ cycloalkyl), heterocyclyl, aryl, heteroaryl or a bond, where each of the (C₃-C₇ cycloalkyl), heterocyclyl, aryl or heteroaryl is unsubstituted or substituted, and the (C₃-C₇ cycloalkyl) and/or heterocyclyl is optionally part of a bridged, fused or spiro ring system;
R₁₄ is H, CR₁₅R₁₆R₁₇, OR₁₇, SR₁₇, NR₁₆R₁₇, C₁-C₆ alkyl, C₁-C₆ fluoroalkyl, cycloalkyl, heterocyclyl, aryl or heteroaryl, where each of the C₁-C₆ alkyl, C₁-C₆ fluoroalkyl, cycloalkyl, heterocyclyl, aryl or heteroaryl is unsubstituted or substituted;
each of R₁₅ and R₁₆ is independently H or C₁-C₃ alkyl; and
each R₁₇ is independently H, C₁-C₆ alkyl, C₁-C₆ fluoroalkyl, cycloalkyl, heterocyclyl, aryl or heteroaryl, where each of the C₁-C₆ alkyl, C₁-C₆ fluoroalkyl, cycloalkyl, heterocyclyl, aryl or heteroaryl is unsubstituted or substituted; or alternatively, R₁₆ and R₁₇ together with the attached nitrogen atom may form a substituted or unsubstituted ring,
with the proviso that when R₅ is -L₈-L₉-L₁₀-L₁₁-L₁₂-R₁₄, at least one of L₈, L₉, L₁₀, L₁₁, L₁₂ and R₁₄ is a carbon-containing moiety and R₅ is directly attached to the (isoquinolone) core structure by a carbon atom;
or R₅ is
a non-aromatic N-linked heterocyclic ring 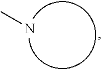
where the heterocyclic ring is substituted or unsubstituted, optionally contains one or more additional ring atoms selected from N, O, Si and S, and is optionally part of a bridged, fused or spiro ring system.

41. The compound according to claim 40 or a solvate, enantiomer, diastereomer, tautomer, polymorph or isotope-labeled compound, or a pharmaceutically acceptable salt thereof, wherein R₅ is —(NR₁₀)-L₁-L₂-L₃-L₄-L₅-L₆-L₇-R₉, where L₁ to L₇, R₉ and R₁₀ are as defined.

42. The compound according to claim 40 or a solvate, enantiomer, diastereomer, tautomer, polymorph or isotope-labeled compound, or a pharmaceutically acceptable salt thereof, wherein R₅ is a non-aromatic N-linked heterocyclyl ring 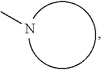
where the heterocyclyl ring is substituted or unsubstituted, optionally contains one or more additional ring atoms selected from N, O, Si and S, and is optionally part of a bridged, fused or spiro ring system.

43. The compound according to claim 40 or a solvate, enantiomer, diastereomer, tautomer, polymorph or isotope-labeled compound, or a pharmaceutically acceptable salt thereof, wherein R₁ is aryl, where the aryl is unsubstituted or substituted, R₂ is CH₃ and R₃ is H.

44. The compound according to claim 40 or a solvate, enantiomer, diastereomer, tautomer, polymorph or isotope-labeled compound, or a pharmaceutically acceptable salt thereof, wherein R₁ is heteroaryl, where the heteroaryl is unsubstituted or substituted, R₂ is CH₃ and R₃ is H.

45. The compound according to claim 40 or a solvate, enantiomer, diastereomer, tautomer, polymorph or isotope-labeled compound, or a pharmaceutically acceptable salt thereof, wherein R₁ is aryl, where the aryl is unsubstituted or substituted, R₂ is CH₃, R₃ is H and R₈ is H.

46. The compound according to claim 40 or a solvate, enantiomer, diastereomer, tautomer, polymorph or isotope-labeled compound, or a pharmaceutically acceptable salt thereof, wherein R₁ is heteroaryl, where the heteroaryl is unsubstituted or substituted, R₂ is CH₃, R₃ is H and R₈ is H.

47. The compound according to claim 40 or a solvate, enantiomer, diastereomer, tautomer, polymorph or isotope-labeled compound, or a pharmaceutically acceptable salt thereof, wherein R₁ is aryl, where the aryl is unsubstituted or substituted, R₂ is CH₃, R₃ is H, R₈ is H and R₆ is CH₃.

48. The compound according to claim 40 or a solvate, enantiomer, diastereomer, tautomer, polymorph or isotope-labeled compound, or a pharmaceutically acceptable salt thereof, wherein R₁ is heteroaryl, where the heteroaryl is unsubstituted or substituted, R₂ is CH₃, R₃ is H, R₈ is H and R₆ is CH₃.

49. The compound according to claim 40 or a solvate, enantiomer, diastereomer, tautomer, polymorph or isotope-labeled compound, or a pharmaceutically acceptable salt thereof, wherein R₁ is aryl, where the aryl is unsubstituted or substituted, R₂ is CH₃, R₃ is H and R₅ is —(NR₁₀)-L₁-L₂-L₃-L₄-L₅-L₆-L₇-R₉.

50. The compound according to claim 40 or a solvate, enantiomer, diastereomer, tautomer, polymorph or isotope-labeled compound, or a pharmaceutically acceptable salt thereof, wherein R₁ is aryl, where the aryl is unsubstituted or substituted, R₂ is CH₃, R₃ is H, R₅ is —(NR₁₀)-L₁-L₂-L₃-L₄-L₅-L₆-L₇-R₉ and R₈ is H.

51. The compound according to claim 40 or a solvate, enantiomer, diastereomer, tautomer, polymorph or isotope-labeled compound, or a pharmaceutically acceptable salt thereof, wherein the compound of Formula (1) is a compound of Formula (2) 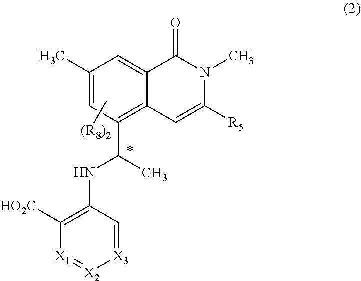
or a solvate, enantiomer, diastereomer, tautomer, polymorph or isotope-labeled compound, or a pharmaceutically acceptable salt thereof,
wherein:
each of X₁, X₂ and X₃ is independently N, CH or substituted C;
R₅ and R₈ are defined as in the compound of Formula (1), and
the carbon marked with * is a chiral center and exists as a (R)- and (S)-racemic mixture or as either the (R)- or (S)-enantiomer.

52. A pharmaceutical composition comprising the compound of claim 40 or a solvate, enantiomer, diastereomer, tautomer, polymorph or isotope-labeled compound, or a pharmaceutically acceptable salt thereof and a pharmaceutically acceptable carrier.

53. A pharmaceutical composition comprising the compound of claim 51 or a solvate, enantiomer, diastereomer, tautomer, polymorph or isotope-labeled compound, or a pharmaceutically acceptable salt thereof and a pharmaceutically acceptable carrier.

54. A method of treating a disease in which PI3K activity is implicated in a subject in need of such treatment, the method comprising administering to the subject a therapeutically effective amount of the compound of claim 40 or a solvate, enantiomer, diastereomer, tautomer, polymorph or isotope-labeled compound, or a pharmaceutically acceptable salt thereof.

55. A method of treating a disease in which PI3K activity is implicated in a subject in need of such treatment, the method comprising administering to the subject a therapeutically effective amount of the compound of claim 51 or a solvate, enantiomer, diastereomer, tautomer, polymorph or isotope-labeled compound, or a pharmaceutically acceptable salt thereof.
